(19) United States
(12) Patent Application Publication
(10) Pub. No.: US 2026/0256697 A1
(43) Pub. Date: Sep. 3, 2026

(54) VIRAL PARTICLES WITH FUSOGEN DISPLAY AND RELATED COMPOSITIONS AND METHODS

(71) Applicant: Sana Biotechnology, Inc., Seattle, WA (US)

(72) Inventors: Kyle Marvin TRUDEAU, Boston, MA (US); Luca BIASCO, Boston, MA (US)

(73) Assignee: Sana Biotechnology, Inc., Seattle, WA (US)

(21) Appl. No.: 19/334,401

(22) Filed: Sep. 19, 2025

Related U.S. Application Data
(63) Continuation of application No. PCT/US2025/017875, filed on Feb. 28, 2025.
(60) Provisional application No. 63/560,612, filed on Mar. 1, 2024.
(60) Provisional application No. 63/635,585, filed on Apr. 17, 2024.

(51) Int. Cl. A61K 9/1271; A61K 31/7088; A61K 38/16; A61K 38/46; A61K 47/08; A61K 48/00; C12N 5/071; C12N 15/11

(52) U.S. Cl. CPC A61K 9/1271; A61K 31/7088; A61K 38/162; A61K 38/465; A61K 47/08; A61K 48/0025; C12N 5/0602; C12N 15/11

(57) ABSTRACT

Provided herein are lipid particles containing a viral envelope attachment protein, such as derived from paramyxovirus or baboon endogenous virus (BaEV). In some embodiments, the lipid particles also contain a codisplay protein, such as a transduction adjuvant or an immune stimulating protein. Also provided are such lipid particles that are viral vectors, such as lentiviral vectors or lentiviral-like particles. Also provided are producer cells and compositions containing such lipid particles and methods of making and using the lipid particles.

### CROSS-REFERENCE TO RELATED APPLICATIONS

This application is a continuation of International Application No. PCT/US2025/017875, entitled “VIRAL PARTICLES WITH FUSOGEN DISPLAY AND RELATED COMPOSITIONS AND METHODS,” filed on Feb. 28, 2025, which claims priority to U.S. Provisional Patent Application No. 63/560,612 filed on Mar. 1, 2024, entitled “VIRAL PARTICLES WITH FUSOGEN DISPLAY AND RELATED COMPOSITIONS AND METHODS”, and U.S. Provisional Patent Application No. 63/635,585 filed on Apr. 17, 2024, entitled “VIRAL PARTICLES WITH FUSOGEN DISPLAY AND RELATED COMPOSITIONS AND METHODS”, each of which are hereby incorporated by reference in its entirety.

### REFERENCE TO AN ELECTRONIC SEQUENCE LISTING

The present application is being filed along with a Sequence Listing in electronic format. The Sequence Listing is provided as a file entitled 186152009901_Seq created Sep. 18, 2025, which is 1,164,085 bytes in size. The information in the electronic format of the Sequence Listing is incorporated by reference in its entirety.

### FIELD

The present disclosure relates to lipid particles containing certain viral envelope attachment protein, such as derived from paramyxovirus or baboon endogenous virus (BaEV). In some embodiments, the lipid particles also contain a codisplay protein, such as a transduction adjuvant or an immune stimulating protein. Also provided are such lipid particles that are viral vectors, such as lentiviral vectors or lentiviral-like particles. Also provided are producer cells and compositions containing such lipid particles and methods of making and using the lipid particles.

### BACKGROUND

Lipid particles, including viral-based particles like virus-like particles and viral vectors such as lentiviral particles, are commonly used for delivery of exogenous agents to cells. For various particles, such as lentiviral vector particles, the host range can be altered by pseudotyping with at least one retargeted attachment protein that is or comprises a heterologous envelope protein or modified envelope protein. The efficient preparation and production of particles with certain retargeted pseudotyped envelope proteins to produce a higher titer and with efficient transduction efficiency of target cells are needed. The provided disclosure addresses this need.

### SUMMARY

Provided herein is a lipid particle, comprising (a) a retargeted attachment protein comprising (i) a paramyxovirus envelope attachment protein; and (ii) a targeting moiety directed to a target molecule expressed on the surface of a target cell, (b) a co-display protein; and (c) at least one paramyxovirus fusion (F) protein; and wherein the co-display protein increases the potency and/or specificity of the lipid particle as compared to a lipid particle not comprising the co-display protein, wherein the protein in (a), (b) and (c) are exposed on the outside of the lipid bilayer/embedded in the lipid bilayer.

Provided herein is a lipid particle, comprising (a) a retargeted attachment protein comprising (i) a paramyxovirus envelope attachment protein; and (ii) a targeting moiety directed to a target molecule expressed on the surface of a target cell, (b) a co-display protein for increasing the potency and/or specificity of the lipid particle as compared to a lipid particle not comprising the co-display protein; and (c) at least one paramyxovirus fusion (F) protein; and wherein the protein in (a), (b) and (c) are exposed on the outside of the lipid bilayer/embedded in the lipid bilayer.

Provided herein is a lipid particle, comprising: (a) a retargeted attachment protein comprising (i) a paramyxovirus envelope attachment protein; and (ii) a targeting moiety directed to a first target molecule expressed on the surface of a target cell, (b) a co-display protein comprising a binding means for specifically interacting with a second target molecule expressed on the surface of the target cell; and (c) at least one paramyxovirus fusion (F) protein; and wherein the protein in (a), (b) and (c) are exposed on the outside of the lipid bilayer/embedded in the lipid bilayer.

Provided herein is a lipid particle, comprising: (a) a Baboon Endogenous Virus (BaEV) fusogen or functional variant thereof, and (b) a co-display protein comprising a binding means for specifically interacting with a target molecule expressed on the surface of the target cell; and wherein the protein in (a) and (b) are exposed on the outside of the lipid bilayer/embedded in the lipid bilayer.

In some of any of the provided embodiments, the co-display protein is an immune stimulating protein and/or is a transduction adjuvant.

Provided herein is a lipid particle, comprising (a) a retargeted attachment protein comprising (i) a paramyxovirus envelope attachment protein; and (ii) a targeting moiety directed to a target molecule expressed on the surface of a target cell, (b) a co-display protein selected from an immune stimulating protein and a transduction adjuvant; and (c) at least one paramyxovirus fusion (F) protein; and wherein the protein in (a), (b) and (c) are embedded in the lipid bilayer.

In some of any of the provided embodiments, the co-display protein is selected from the group comprising of a glycoprotein, transmembrane protein, receptor, integrin, antibody or antigen-binding fragment, an engineered binding domain, a nanobody, a DARPin, an Aptamer, an Affimer, an Affibody, a Knottin, an Avimer, a Monobody, an Anticalin, a Fynomer, and a targeting peptide. In some of any of the provided embodiments, the co-display protein increases specificity to the target cell. In some of any of the provided embodiments, the co-display protein increases specificity to a target cell selected from the group consisting of tumor-infiltrating lymphocytes, T cells, neoplastic or tumor cells, virus-infected cells, stem cells, central nervous system (CNS) cells, hematopoietic stem cells (HSCs), and liver cells. In some of any of the provided embodiments, the co-display protein increases specificity to a target cell selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some of any of the provided embodiments, the co-display protein increases specificity to a target cell that is a hematopoietic stem cell. In some of any of the provided embodiments, the cell surface molecule is selected from CD34, CD105, or CD117. In some of any of the provided embodiments, the co-display protein increases specificity to a target cell that is a T cell. In some of any of the provided embodiments, the cell surface molecule is selected from CD3, CD4, CD7 CD8, ASCT2, CD110, CD133, CD146, CD164, CD46, CD49f, CD90, EPCR, and ITGA3. In some of any of the provided embodiments, the co-display protein increases specificity to a target cell that is a hepatocyte. In some of any of the provided embodiments, the cell surface molecule is selected from ASGR1, ASGR2 and TM4SF5.

In some of any of the provided embodiments, the co-display protein binds to a different protein than the targeting moiety of the retargeted attachment protein. In some of any of the provided embodiments, the co-display protein does not include a paramyxovirus envelope attachment protein or a fragment thereof. In some of any of the provided embodiments, the co-display protein is an immune stimulating protein and the immune stimulating protein stimulates a T cell activity or function. In some of any of the provided embodiments, the immune stimulating protein comprises (i) an extracellular domain, (ii) a linker, and (iii) an anchor. In some of any of the provided embodiments, the extracellular domain of the immune stimulating protein binds to a T cell stimulatory receptor. In some of any of the provided embodiments, the T cell stimulatory receptor is selected from CD3, CD2, CD28, CD27. In some of any of the provided embodiments, the extracellular domain of the immune stimulating protein binds to CD3. In some of any of the provided embodiments, the extracellular domain is selected from the group comprising an antibody or antigen-binding fragment, an engineered binding domain, a nanobody, a DARPin, an Aptamer, an Affimer, an Affibody, a Knottin, an Avimer, a Monobody, an Anticalin, a Fynomer, and a targeting peptide. In some of any of the provided embodiments, the extracellular domain of the immune stimulating protein is an anti-CD3 antibody or single chain fragment thereof.

In some of any of the provided embodiments, the co-display protein is a transduction adjuvant that increases transduction efficiency of the lipid particle as compared to a lipid particle that does not have a co-display protein comprising a transduction adjuvant. In some of any of the provided embodiments, the co-display protein is a transduction adjuvant that binds to a surface molecule on target cells to enhance transduction. In some of any of the provided embodiments, the transduction adjuvant comprises (i) an extracellular domain, (ii) a linker, and (iii) an anchor. In some of any of the provided embodiments, the extracellular domain of the transduction adjuvant binds an integrin binding domain. In some of any of the provided embodiments, the extracellular domain of the transduction adjuvant comprises the extracellular domain of fibronectin, retronectin, or a functional variant thereof.

In some of any of the provided embodiments, the linker is derived from a human protein. In some of any of the provided embodiments, the linker is a derived from human CD8a, IgG3 hinge, IgG1 hinge, IgG4 heavy chain, IgG4, or CD28. In some of any of the provided embodiments, the linker is modified to remove a cysteine residue. In some of any of the provided embodiments, the linker is rigid or wherein the linker is flexible.

In some of any of the provided embodiments, the anchor is a peptide anchor that is a transmembrane domain. In some of any of the provided embodiments, the anchor is a transmembrane domain that is a transmembrane domain of a protein selected from PDGFR, VSV-G, CD8α, ICAM1, HLA-DRB, CD49d, or Transferrin. In some of any of the provided embodiments, the anchor is a GPI lipid anchor.

In some of any of the provided embodiments, the targeting moiety is selected from the group consisting of an antibody or antigen-binding fragment, an engineered binding domain, a nanobody, a DARPin, an Aptamer, an Affimer, an Affibody, a Knottin, an Avimer, a Monobody, an Anticalin, a Fynomer, and a targeting peptide. In some of any of the provided embodiments, the targeting moiety binds to a cell surface molecule present on a target cell. In some of any of the provided embodiments, the cell surface molecule is a protein, glycan, or lipid. In some of any of the provided embodiments, the target cell is selected from the group consisting of tumor-infiltrating lymphocytes, T cells, neoplastic or tumor cells, virus-infected cells, stem cells, central nervous system (CNS) cells, hematopoietic stem cells (HSCs), and liver cells. In some of any of the provided embodiments, the target cells is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell. In some of any of the provided embodiments, the target is a hematopoietic stem cell. In some of any of the provided embodiments, the cell surface molecule is selected from CD34, CD105, or CD117. In some of any of the provided embodiments, the target cell is a T cell. In some of any of the provided embodiments, the cell surface molecule is selected from the group consisting of CD3, CD4, CD7 CD8, ASCT2, CD110, CD133, CD146, CD164, CD46, CD49f, CD90, EPCR, and ITGA3. In some of any of the provided embodiments, the target cell is a hepatocyte. In some of any of the provided embodiments, the cell surface molecule is selected from the group consisting of ASGR1, ASGR2 and TM4SF5. In some of any of the provided embodiments, the targeting moiety is selected from the group consisting of a single domain antibody or a single chain variable fragment (scFv). In some of any of the provided embodiments, the single domain antibody is a VHH.

In some of any of the provided embodiments, the retargeted attachment protein is linked to a (i) first targeting moiety directed to a first target molecule expressed on the surface of a target cell, and a (ii) second targeting moiety directed to a second target molecule expressed on the surface of a target cell. In some of any of the provided embodiments, the paramyxovirus envelope attachment protein of the retargeted attachment protein is a first paramyxovirus envelope attachment protein and the lipid particle further comprises a second paramyxovirus envelope attachment protein. In some of any of the provided embodiments, the second paramyxovirus envelope attachment protein is linked to the first paramyxovirus envelope attachment protein in tandem. In some of any of the provided embodiments, the retargeted attachment protein comprising the first paramyxovirus envelope attachment protein and the second paramyxovirus envelope attachment protein are each separately exposed the outside of the lipid bilayer/embedded in the lipid bilayer. In some of any of the provided embodiments, the second paramyxovirus attachment protein is a retargeted attachment protein. In some of any of the provided embodiments, the second retargeted attachment protein comprises (i) a second paramyxovirus envelope attachment protein; and (ii) a second targeting moiety directed to a target molecule expressed on the surface of a target cell. In some of any of the provided embodiments, the targeting moiety is selected from the group consisting of an antibody or antigen-binding fragment, an engineered binding domain, a nanobody, a DARPin, an Aptamer, an Affimer, an Affibody, a Knottin, an Avimer, a Monobody, an Anticalin, a Fynomer, and a targeting peptide. In some of any of the provided embodiments, the target cell is selected from the group consisting of tumor-infiltrating lymphocytes, T cells, neoplastic or tumor cells, virus-infected cells, stem cells, central nervous system (CNS) cells, hematopoietic stem cells (HSCs), and liver cells. In some of any of the provided embodiments, the target cells is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell. In some of any of the provided embodiments, the target is a hematopoietic stem cell. In some of any of the provided embodiments, the cell surface molecule is selected from CD34, CD105, or CD117. In some of any of the provided embodiments, the target cell is a T cell. In some of any of the provided embodiments, the cell surface molecule is selected from the group consisting of CD3, CD4, CD7 CD8, ASCT2, CD110, CD133, CD146, CD164, CD46, CD49f, CD90, EPCR, and ITGA3. In some of any of the provided embodiments, the target cell is a hepatocyte. In some of any of the provided embodiments, the cell surface molecule is selected from the group consisting of ASGR1, ASGR2 and TM4SF5. In some of any of the provided embodiments, the targeting moiety is selected from the group consisting of a single domain antibody or a single chain variable fragment (scFv). In some of any of the provided embodiments, the single domain antibody is a VHH.

In some of any of the provided embodiments, the BaEV envelope glycoprotein is a truncated BaEV envelope glycoprotein comprising a cytoplasmic tail with a partial fusion inhibitory R peptide relative to the inhibitory R peptide of a wild-type BaEV envelope glycoprotein, wherein the partial fusion inhibitory R peptide comprises at least one amino-terminal amino acid but less than the full length of the inhibitory R peptide of the wild-type BaEV envelope glycoprotein. In some of any of the provided embodiments, the BaEV envelope glycoprotein is a truncated BaEV envelope glycoprotein comprising a cytoplasmic tail with a partial fusion inhibitory R peptide relative to a wild-type BaEV envelope glycoprotein, wherein the cytoplasmic tail is 25 amino acids in length and contains 8 contiguous amino-terminal acids of the inhibitory R peptide (R+8) of the full length inhibitory R peptide of wild-type BaEV envelope glycoprotein. In some of any of the provided embodiments, the truncated BaEV glycoprotein comprises: (i) a glycoprotein 70 (g70) subunit or a biologically active portion thereof, and (ii) a portion of the glycoprotein p20E (p20E) subunit comprising the cytoplasmic tail with the partial inhibitory R peptide. In some of any of the provided embodiments, the BaEV envelope glycoprotein binds an ASCT-2 or ASCT-1 receptor. In some of any of the provided embodiments, the truncated BaEV envelope glycoprotein is truncated relative to SEQ ID NO: 622 and lacks up to 16 contiguous amino acids from the C-terminal cytoplasmic tail of SEQ ID NO: 622. In some of any of the provided embodiments, the partial fusion inhibitory R peptide is set forth as amino acids 1 to 8 of SEQ ID NO: 623, optionally wherein the cytoplasmic tail is set forth in SEQ ID NO: 624 (R+8). In some of any of the provided embodiments, the truncated BaEV envelope glycoprotein is set forth in SEQ ID NO: 625.

Provided herein is a lipid particle, comprising a variant Baboon Endogenous Virus (BaEV) fusogen wherein the fusogen is exposed on the outside of the lipid bilayer/embedded in the lipid bilayer. In some of any of the provided embodiments, the variant BaEV envelope glycoprotein has the amino acid sequence set forth in SEQ ID NO: 795 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:795. In some of any of the provided embodiments, the variant BaEV envelope glycoprotein has the amino acid sequence set forth in SEQ ID NO: 796 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:796. In some of any of the provided embodiments, the variant BaEV envelope glycoprotein has the amino acid sequence set forth in SEQ ID NO: 797 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:797.

In some embodiments, the BaEV Baboon Endogenous Virus (BaEV) is a BaEVTR fusogen. In some embodiments, the BaEV Baboon Endogenous Virus (BaEV) fusogen or functional variant thereof comprises a protease site modification. In some embodiments, the protease site modification comprises modifying a MLV-A cleavage site to a MA/CA cleavage site.

In some of any of the provided embodiments, the paramyxovirus envelope attachment protein is an envelope attachment protein from a Nipah virus, Hendra virus, or Measles virus, or is a variant or biologically active portion thereof of any of the foregoing. In some of any of the provided embodiments, the paramyxovirus envelope attachment protein is a wild-type paramyxovirus G protein, H protein or HN protein or is a variant or biologically active portion of any of the foregoing. In some of any of the provided embodiments, the paramyxovirus envelope attachment protein is a wild-type Nipah virus G (NiV-G) protein or is a variant or biologically active portion of a NiV-G. In some of any of the provided embodiments, the paramyxovirus envelope attachment protein is a variant paramyxovirus envelope attachment protein. In some of any of the provided embodiments, the variant paramyxovirus envelope attachment protein comprises one or more mutations that reduces native tropism relative to the wild-type paramyxovirus envelope attachment protein not comprising the one or more mutations. In some of any of the provided embodiments, the paramyxovirus envelope attachment protein is a variant NiV-G that is a variant or a biologically active portion of a wild-type NiV-G. In some of any of the provided embodiments, the variant is a variant NiV-G that is a variant of a wild-type Nipah virus G (NiV-G) protein or a biologically active portion thereof. In some of any of the provided embodiments, the variant NiV-G is truncated by up to 40 contiguous amino acids at or near the N-terminus of the wild-type NiV-G set forth in SEQ ID NO:1. In some of any of the provided embodiments, the variant NiV-G has a truncation of amino acids 2-34 of the wild-type NiV-G set forth in SEQ ID NO:1. In some of any of the provided embodiments, the variant NiV-G exhibits reduced binding to Ephrin B2 or Ephrin B3. In some of any of the provided embodiments, the variant NiV-G comprises: one or more amino acid substitutions corresponding to amino acid substitutions selected from the group consisting of E501A, W504A, Q530A and E533A with reference to numbering set forth in SEQ ID NO:1. In some of any of the provided embodiments, the variant NiV-G comprises amino acid substitutions E501A, W504A, Q530A and E533A with reference to numbering set forth in SEQ ID NO:1. In some of any of the provided embodiments, the variant NiV-G has the amino acid sequence set forth in SEQ ID NO: 228 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:228. In some of any of the provided embodiments, the variant NiV-G has the amino acid sequence set forth in SEQ ID NO:228.

In some of any of the provided embodiments, the particle further comprises an additional attachment protein that is a paramyxovirus envelope attachment protein that is a variant NiV-G that is a variant or a biologically active portion of a wild-type NiV-G. In some of any of the provided embodiments, the variant NiV-G exhibits reduced binding to Ephrin B2 or Ephrin B3. In some of any of the provided embodiments, the variant NiV-G comprises: one or more amino acid substitutions corresponding to amino acid substitutions selected from the group consisting of E501A, W504A, Q530A and E533A with reference to numbering set forth in SEQ ID NO:1.

In some of any of the provided embodiments, the at least one paramyxovirus fusion (F) protein is an F protein from a henipavirus or is a biologically active portion thereof or variant thereof. In some of any of the provided embodiments, the henipavirus is a Hendra virus. In some of any of the provided embodiments, the henipavirus is a Nipah virus. In some of any of the provided embodiments, the paramyxovirus F protein is a wild-type NiV-F protein or a variant or a biologically active portion thereof. In some of any of the provided embodiments, the paramyxovirus F protein is a variant NiV-F that is a variant or a biologically active portion of a wild-type NiV-F protein. In some of any of the provided embodiments, the variant NiV-F is truncated by up to 22 contiguous amino acids the at the C-terminus of the wild-type NiV-F set forth in SEQ ID NO:235, optionally not include the initial methionine. In some of any of the provided embodiments, the variant NiV-F protein is a truncated NiV-F that lacks amino acids 525-546 of SEQ ID NO:235. In some of any of the provided embodiments, the variant NiV-F has the amino acid sequence set forth in SEQ ID NO: 227 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:227. In some of any of the provided embodiments, the variant NiV-F has the amino acid sequence set forth in SEQ ID NO:227. In some of any of the provided embodiments, the paramyxovirus F protein is an F0 precursor or is a proteolytically cleaved form thereof comprising F1 and F2 subunits. In some of any of the provided embodiments, the proteolytically cleaved form is a cathepsin L cleavage product.

In some of any of the provided embodiments, the targeting moiety and the paramyxovirus envelope attachment protein or biologically active portion thereof is attached via a linker. In some of any of the provided embodiments, the linker is a peptide linker. In some of any of the provided embodiments, the peptide linker is 2 to 65 amino acids in length. In some of any of the provided embodiments, the peptide linker is a flexible linker that comprises GS, GGS, GGGGS, GGGGGS or combinations thereof. In some of any of the provided embodiments, the peptide linker is selected from: (GGS)n, wherein n is 1 to 10; (GGGGS)n, wherein n is 1 to 10; or (GGGGGS)n, wherein n is 1 to 6. In some of any of the provided embodiments, the paramyxovirus fusion (F) protein exhibits fusogenic activity with a target cell upon binding of at least one paramyxovirus envelope attachment protein o to the target molecule on the target cell.

In some of any of the provided embodiments, the lipid particle comprises a viral nucleic acid. In some of any of the provided embodiments, the viral nucleic acid comprises one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3). In some of any of the provided embodiments, the lipid particle is a viral vector. In some of any of the provided embodiments, the lipid particle is a retroviral vector. In some of any of the provided embodiments, the lipid particle is a lentiviral vector. In some of any of the provided embodiments, the lipid particle is devoid of viral genomic DNA. In some of any of the provided embodiments, the lipid particle is a viral-like particle. In some of any of the provided embodiments, the lipid particle is a retroviral-like particle. In some of any of the provided embodiments, the lipid particle is a lentiviral-like particle. In some of any of the provided embodiments, the lentiviral vector or lentiviral-like particle is derived from HIV. In some of any of the provided embodiments, the Baboon Endogenous Virus (BaEV) fusogen or functional variant thereof is a BaEVTR fusogen. In some of any of the provided embodiments, the BaEV Baboon Endogenous Virus (BaEV) fusogen or functional variant thereof comprises a protease site modification. In some of any of the provided embodiments, the protease site modification comprises modifying a MLV-A cleavage site to a MA/CA cleavage site.

In some of any of the provided embodiments, the lipid particle is produced as a preparation with increased titer compared to a reference lipid particle preparation that is similarly produced but with only the first retargeted attachment protein. In some of any of the provided embodiments, the titer is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.

In some of any of the provided embodiments, the particle further comprises an exogenous agent for delivery to a target cell. In some of any of the provided embodiments, the exogenous agent is present in the lumen. In some of any of the provided embodiments, the exogenous agent is a protein or a nucleic acid, optionally wherein the nucleic acid is a DNA or RNA. In some of any of the provided embodiments, the exogenous agent is a nucleic acid encoding a cargo for delivery to the target cell. In some of any of the provided embodiments, the exogenous agent is or encodes a therapeutic agent, a diagnostic agent or a genome-modifying enzyme. In some of any of the provided embodiments, the exogenous agent encodes a membrane protein, optionally wherein the membrane protein is an antigen receptor for targeting cells expressed by or associated with a disease or condition. In some of any of the provided embodiments, the membrane protein is a chimeric antigen receptor (CAR). In some of any of the provided embodiments, the exogenous agent is a nucleic acid comprising a payload gene for correcting a genetic deficiency, optionally a genetic deficiency in the target cell, optionally wherein the genetic deficiency is associated with a liver cell or a hepatocyte. In some of any of the provided embodiments, binding of the paramyxovirus envelope attachment protein or biologically active portion thereof to a target molecule expressed on the surface of a target cell mediates fusion of the particle with the target cell and delivery of the exogenous agent to the target cell. In some of any of the provided embodiments, at or greater than 10%, 20%, 30%, 40%, 50%, 60% of the target cells are delivered the exogenous agent. In some of any of the provided embodiments, delivery of the exogenous cell to the target cell is increased compared to a reference particle preparation that is similarly produced but with only a retargeted attachment protein (e.g., no co-display protein). In some of any of the provided embodiments, the delivery to the target cell is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.

Provided herein is a producer cell comprising (a) a nucleic acid encoding a retargeted attachment protein comprising a paramyxovirus envelope attachment protein; and (i) a targeting moiety directed to a target molecule expressed on the surface of a target cell, (b) a nucleic acid encoding a co-display protein, and (c) a nucleic acid encoding at least one paramyxovirus F protein.

Provided herein is a producer cell comprising (a) a nucleic acid encoding a retargeted attachment protein comprising a Baboon Endogenous Virus (BaEV) fusogen or functional variant thereof expressed on the surface of a target cell, and (b) a nucleic acid encoding a co-display protein.

In some of any of the provided embodiments, the cell further comprises a viral nucleic acid(s). In some of any of the provided embodiments, the viral nucleic acid(s) are lentiviral nucleic acids. In some of any of the provided embodiments, the cell is a mammalian cell. In some of any of the provided embodiments, the producer cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In some of any of the provided embodiments, the producer cell comprises 293T cells.

In some of any of the provided embodiments, the viral nucleic acid(s) lacks one or more genes involved in viral replication. In some of any of the provided embodiments, the viral nucleic acid comprises a nucleic acid encoding a viral packaging protein selected from one or more of Gag, Pol, Rev and Tat. In some of any of the provided embodiments, the viral nucleic acid comprises: one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3). In some of any of the provided embodiments, the co-display protein increases specificity to the target cell. In some of any of the provided embodiments, the co-display protein increases specificity to a target cell selected from the group consisting of tumor-infiltrating lymphocytes, T cells, neoplastic or tumor cells, virus-infected cells, stem cells, central nervous system (CNS) cells, hematopoietic stem cells (HSCs), and liver cells. In some of any of the provided embodiments, the co-display protein increases specificity to a target cell selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell. In some of any of the provided embodiments, the co-display protein increases specificity to a target cell that is a hematopoietic stem cell. In some of any of the provided embodiments, the cell surface molecule is selected from CD34, CD105, or CD117. In some of any of the provided embodiments, the co-display protein increases specificity to a target cell that is a T cell. In some of any of the provided embodiments, the cell surface molecule is selected from CD3, CD4, CD7 CD8, ASCT2, CD110, CD133, CD146, CD164, CD46, CD49f, CD90, EPCR, and ITGA3. In some of any of the provided embodiments, the co-display protein increases specificity to a target cell that is a hepatocyte. In some of any of the provided embodiments, the cell surface molecule is selected from ASGR1, ASGR2 and TM4SF5. In some of any of the provided embodiments, the co-display protein binds to a different protein than the targeting moiety of the retargeted attachment protein. In some of any of the provided embodiments, the co-display protein does not include a paramyxovirus envelope attachment protein or a fragment thereof.

In some of any of the provided embodiments, the co-display protein is an immune stimulating protein and the immune stimulating protein stimulates a T cell activity or function. In some of any of the provided embodiments, the immune stimulating protein comprises (i) an extracellular domain (ii) a linker and (iii) an anchor. In some of any of the provided embodiments, the extracellular domain of the immune stimulating protein binds to a T cell stimulatory receptor. In some of any of the provided embodiments, the T cell stimulatory receptor is selected from CD3, CD2, CD28, CD27. In some of any of the provided embodiments, the extracellular domain of the immune stimulating protein binds to CD3. In some of any of the provided embodiments, the extracellular domain is selected from the group comprising an antibody or antigen-binding fragment, an engineered binding domain, a nanobody, a DARPin, an Aptamer, an Affimer, an Affibody, a Knottin, an Avimer, a Monobody, an Anticalin, a Fynomer, and a targeting peptide. In some of any of the provided embodiments, the extracellular domain of the immune stimulating protein is an anti-CD3 antibody or single chain fragment thereof. In some of any of the provided embodiments, the co-display protein is a transduction adjuvant that increases transduction efficiency of the lipid particle as compared to a lipid particle that does not have a co-display protein comprising a transduction adjuvant. In some of any of the provided embodiments, the co-display protein is a transduction adjuvant that binds to a surface molecule on target cells to enhance transduction.

In some of any of the provided embodiments, the transduction adjuvant comprises (i) an extracellular domain (ii) a linker and (iii) an anchor. In some of any of the provided embodiments, the extracellular domain of the transduction adjuvant binds an integrin binding domain. In some of any of the provided embodiments, the extracellular domain of the transduction adjuvant comprises the extracellular domain of fibronectin, retronectin, or a functional variant thereof. In some of any of the provided embodiments, the linker is derived from a human protein. In some of any of the provided embodiments, the linker is a derived from human CD8α, IgG3 hinge, IgG1 hinge, IgG4 heavy chain, IgG4, or CD28. In some of any of the provided embodiments, the linker is modified to remove a cysteine residue. In some of any of the provided embodiments, the linker is rigid or wherein the linker is flexible. In some of any of the provided embodiments, the anchor is a peptide anchor that is a transmembrane domain. In some of any of the provided embodiments, the anchor is a transmembrane domain that is a transmembrane domain of a protein selected from PDGFR, VSV-G, CD8α, ICAM1, HLA-DRB, CD49d, or Transferrin. In some of any of the provided embodiments, the anchor is a GPI lipid anchor.

In some of any of the provided embodiments, the targeting moiety is selected from the group consisting of an antibody or antigen-binding fragment, an engineered binding domain, a nanobody, a DARPin, an Aptamer, an Affimer, an Affibody, a Knottin, an Avimer, a Monobody, an Anticalin, a Fynomer, and a targeting peptide. In some of any of the provided embodiments, the targeting moiety binds to a cell surface molecule present on a target cell. In some of any of the provided embodiments, the cell surface molecule is a protein, glycan, or lipid. In some of any of the provided embodiments, the target cell is selected from the group consisting of tumor-infiltrating lymphocytes, T cells, neoplastic or tumor cells, virus-infected cells, stem cells, central nervous system (CNS) cells, hematopoietic stem cells (HSCs), and liver cells. In some of any of the provided embodiments, the target cells is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell. In some of any of the provided embodiments, the target is a hematopoietic stem cell. In some of any of the provided embodiments, the cell surface molecule is selected from CD34, CD105, or CD117. In some of any of the provided embodiments, the target cell is a T cell. In some of any of the provided embodiments, the cell surface molecule is selected from the group consisting of CD3, CD4, CD7 CD8, ASCT2, CD110, CD133, CD146, CD164, CD46, CD49f, CD90, EPCR, and ITGA3. In some of any of the provided embodiments, the target cell is a hepatocyte. In some of any of the provided embodiments, the cell surface molecule is selected from the group consisting of ASGR1, ASGR2 and TM4SF5. In some of any of the provided embodiments, the targeting moiety is selected from the group consisting of a single domain antibody or a single chain variable fragment (scFv). In some of any of the provided embodiments, the single domain antibody is a VHH. In some of any of the provided embodiments, the retargeted attachment protein is a first attachment protein and the lipid particle further comprises a second retargeted attachment protein. In some of any of the provided embodiments, the second retargeted attachment protein comprises (i) a second paramyxovirus envelope attachment protein; and (ii) a second targeting moiety directed to a target molecule expressed on the surface of a target cell. In some of any of the provided embodiments, the second targeting moiety is selected from the group consisting of an antibody or antigen-binding fragment, an engineered binding domain, a nanobody, a DARPin, an Aptamer, an Affimer, an Affibody, a Knottin, an Avimer, a Monobody, an Anticalin, a Fynomer, and a targeting peptide. In some of any of the provided embodiments, the target cell is selected from the group consisting of tumor-infiltrating lymphocytes, T cells, neoplastic or tumor cells, virus-infected cells, stem cells, central nervous system (CNS) cells, hematopoietic stem cells (HSCs), and liver cells. In some of any of the provided embodiments, the target cells is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+neuron, a NKG2D+ natural killer cell, a SLC1A3+ astrocyte, a SLC7A10+ adipocyte, or a CD30+ lung epithelial cell. In some of any of the provided embodiments, the target is a hematopoietic stem cell. In some of any of the provided embodiments, the cell surface molecule is selected from CD34, CD105, or CD117. In some of any of the provided embodiments, the target cell is a T cell. In some of any of the provided embodiments, the cell surface molecule is selected from the group consisting of CD3, CD4, CD7 CD8, ASCT2, CD110, CD133, CD146, CD164, CD46, CD49f, CD90, EPCR, and ITGA3. In some of any of the provided embodiments, the target cell is a hepatocyte. In some of any of the provided embodiments, the cell surface molecule is selected from the group consisting of ASGR1, ASGR2 and TM4SF5.

In some of any of the provided embodiments, the targeting moiety is selected from the group consisting of a single domain antibody or a single chain variable fragment (scFv). In some of any of the provided embodiments, the single domain antibody is a VHH. In some of any of the provided embodiments, the BaEV envelope glycoprotein is a truncated BaEV envelope glycoprotein comprising a cytoplasmic tail with a partial fusion inhibitory R peptide relative to the inhibitory R peptide of a wild-type BaEV envelope glycoprotein, wherein the partial fusion inhibitory R peptide comprises at least one amino-terminal amino acid but less than the full length of the inhibitory R peptide of the wild-type BaEV envelope glycoprotein. In some of any of the provided embodiments, the BaEV envelope glycoprotein is a truncated BaEV envelope glycoprotein comprising a cytoplasmic tail with a partial fusion inhibitory R peptide relative to a wild-type BaEV envelope glycoprotein, wherein the cytoplasmic tail is 25 amino acids in length and contains 8 contiguous amino-terminal acids of the inhibitory R peptide (R+8) of the full length inhibitory R peptide of wild-type BaEV envelope glycoprotein. In some of any of the provided embodiments, the truncated BaEV glycoprotein comprises: (i) a glycoprotein 70 (g70) subunit or a biologically active portion thereof, and (ii) a portion of the glycoprotein p20E (p20E) subunit comprising the cytoplasmic tail with the partial inhibitory R peptide. In some of any of the provided embodiments, the BaEV envelope glycoprotein binds an ASCT-2 or ASCT-1 receptor. In some of any of the provided embodiments, the truncated BaEV envelope glycoprotein is truncated relative to SEQ ID NO: 622 and lacks up to 16 contiguous amino acids from the C-terminal cytoplasmic tail of SEQ ID NO: 622. In some of any of the provided embodiments, the partial fusion inhibitory R peptide is set forth as amino acids 1 to 8 of SEQ ID NO: 623, optionally wherein the cytoplasmic tail is set forth in SEQ ID NO: 624 (R+8). In some of any of the provided embodiments, the truncated BaEV envelope glycoprotein is set forth in SEQ ID NO: 625.

In some of any of the provided embodiments, the paramyxovirus envelope attachment protein is an envelope attachment protein from a Nipah virus, Hendra virus, or Measles virus, or is a variant or biologically active portion thereof of any of the foregoing. In some of any of the provided embodiments, the paramyxovirus envelope attachment protein is a wild-type paramyxovirus G protein, H protein or HN protein or is a variant or biologically active portion of any of the foregoing. In some of any of the provided embodiments, the paramyxovirus envelope attachment protein is a wild-type Nipah virus G (NiV-G) protein or is a variant or biologically active portion of a NiV-G. In some of any of the provided embodiments, the paramyxovirus envelope attachment protein is a variant paramyxovirus envelope attachment protein. In some of any of the provided embodiments, the variant paramyxovirus envelope attachment protein comprises one or more mutations that reduces native tropism relative to the wild-type paramyxovirus envelope attachment protein not comprising the one or more mutations. In some of any of the provided embodiments, the paramyxovirus envelope attachment protein is a variant NiV-G that is a variant or a biologically active portion of a wild-type NiV-G. In some of any of the provided embodiments, the variant is a variant NiV-G that is a variant of a wild-type Nipah virus G (NiV-G) protein or a biologically active portion thereof. In some of any of the provided embodiments, the variant NiV-G is truncated by up to 40 contiguous amino acids at or near the N-terminus of the wild-type NiV-G set forth in SEQ ID NO:1. In some of any of the provided embodiments, the variant NiV-G has a truncation of amino acids 2-34 of the wild-type NiV-G set forth in SEQ ID NO:1. In some of any of the provided embodiments, the variant NiV-G exhibits reduced binding to Ephrin B2 or Ephrin B3. In some of any of the provided embodiments, the variant NiV-G comprises: one or more amino acid substitutions corresponding to amino acid substitutions selected from the group consisting of E501A, W504A, Q530A and E533A with reference to numbering set forth in SEQ ID NO:1. In some of any of the provided embodiments, the variant NiV-G comprises amino acid substitutions E501A, W504A, Q530A and E533A with reference to numbering set forth in SEQ ID NO:1. In some of any of the provided embodiments, the variant NiV-G has the amino acid sequence set forth in SEQ ID NO: 228 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:228. In some of any of the provided embodiments, the variant NiV-G has the amino acid sequence set forth in SEQ ID NO:228.

In some of any of the provided embodiments, the particle further comprises an additional attachment protein that is a paramyxovirus envelope attachment protein that is a variant NiV-G that is a variant or a biologically active portion of a wild-type NiV-G. In some of any of the provided embodiments, the variant NiV-G exhibits reduced binding to Ephrin B2 or Ephrin B3. In some of any of the provided embodiments, the variant NiV-G comprises: one or more amino acid substitutions corresponding to amino acid substitutions selected from the group consisting of E501A, W504A, Q530A and E533A with reference to numbering set forth in SEQ ID NO:1.

In some of any of the provided embodiments, the at least one paramyxovirus fusion (F) protein is an F protein from a henipavirus or is a biologically active portion thereof or variant thereof. In some of any of the provided embodiments, the henipavirus is a Hendra virus. In some of any of the provided embodiments, the henipavirus is a Nipah virus. In some of any of the provided embodiments, the paramyxovirus F protein is a wild-type NiV-F protein or a variant or a biologically active portion thereof. In some of any of the provided embodiments, the paramyxovirus F protein is a variant NiV-F that is a variant or a biologically active portion of a wild-type NiV-F protein. In some of any of the provided embodiments, the variant NiV-F is truncated by up to 22 contiguous amino acids the at the C-terminus of the wild-type NiV-F set forth in SEQ ID NO:235, optionally not include the initial methionine. In some of any of the provided embodiments, the variant NiV-F protein is a truncated NiV-F that lacks amino acids 525-546 of SEQ ID NO:235. In some of any of the provided embodiments, the variant NiV-F has the amino acid sequence set forth in SEQ ID NO: 227 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:227. In some of any of the provided embodiments, the variant NiV-F has the amino acid sequence set forth in SEQ ID NO:227.

In some of any of the provided embodiments, the paramyxovirus F protein is an F0 precursor or is a proteolytically cleaved form thereof comprising F1 and F2 subunits. In some of any of the provided embodiments, the proteolytically cleaved form is a cathepsin L cleavage product. In some of any of the provided embodiments, the targeting moiety and the paramyxovirus envelope attachment protein or biologically active portion thereof is attached via a linker. In some of any of the provided embodiments, the linker is a peptide linker. In some of any of the provided embodiments, the peptide linker is 2 to 65 amino acids in length. In some of any of the provided embodiments, the peptide linker is a flexible linker that comprises GS, GGS, GGGGS, GGGGGS or combinations thereof. In some of any of the provided embodiments, the peptide linker is selected from: (GGS)n, wherein n is 1 to 10; (GGGGS)n, wherein n is 1 to 10; or (GGGGGS)n, wherein n is 1 to 6. In some of any of the provided embodiments, the paramyxovirus fusion (F) protein exhibits fusogenic activity with a target cell upon binding of at least one paramyxovirus envelope attachment protein o to the target molecule on the target cell.

In some of any of the provided embodiments, the lipid particle comprises a viral nucleic acid. In some of any of the provided embodiments, the viral nucleic acid comprises one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3). In some of any of the provided embodiments, the lipid particle is a viral vector. In some of any of the provided embodiments, the lipid particle is a retroviral vector. In some of any of the provided embodiments, the lipid particle is a lentiviral vector.

In some of any of the provided embodiments, the lipid particle is devoid of viral genomic DNA. In some of any of the provided embodiments, the particle is a viral-like particle. In some of any of the provided embodiments, the particle is a retroviral-like particle. In some of any of the provided embodiments, the particle is a lentiviral-like particle. In some embodiments, the lentiviral vector or the lentiviral-like particle is derived from HIV. In some embodiments, the BaEV Baboon Endogenous Virus (BaEV) is a BaEVTR fusogen. In some embodiments, the BaEV Baboon Endogenous Virus (BaEV) fusogen or functional variant thereof comprises a protease site modification. In some embodiments, the protease site modification comprises modifying a MLV-A cleavage site to a MA/CA cleavage site. In some of any of the provided embodiments, the lipid particle is produced as a preparation with increased titer compared to a reference lipid particle preparation that is similarly produced but with only the first retargeted attachment protein. In some of any of the provided embodiments, the titer is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.

In some of any of the provided embodiments, the particle further comprises an exogenous agent for delivery to a target cell. In some of any of the provided embodiments, the exogenous agent is present in the lumen. In some of any of the provided embodiments, the exogenous agent is a protein or a nucleic acid, optionally wherein the nucleic acid is a DNA or RNA. In some of any of the provided embodiments, the exogenous agent is a nucleic acid encoding a cargo for delivery to the target cell. In some of any of the provided embodiments, the exogenous agent is or encodes a therapeutic agent, a diagnostic agent or a genome-modifying enzyme. In some of any of the provided embodiments, the exogenous agent encodes a membrane protein, optionally wherein the membrane protein is an antigen receptor for targeting cells expressed by or associated with a disease or condition. In some of any of the provided embodiments, the membrane protein is a chimeric antigen receptor (CAR).

In some of any of the provided embodiments, the exogenous agent is a nucleic acid comprising a payload gene for correcting a genetic deficiency, optionally a genetic deficiency in the target cell, optionally wherein the genetic deficiency is associated with a liver cell or a hepatocyte. In some of any of the provided embodiments, binding of the paramyxovirus envelope attachment protein or biologically active portion thereof to a target molecule expressed on the surface of a target cell mediates fusion of the particle with the target cell and delivery of the exogenous agent to the target cell. In some of any of the provided embodiments, at or greater than 10%, 20%, 30%, 40%, 50%, 60% of the target cells are delivered the exogenous agent. In some of any of the provided embodiments, delivery of the exogenous cell to the target cell is increased compared to a reference particle preparation that is similarly produced but with only a retargeted attachment protein (e.g., no co-display protein). In some of any of the provided embodiments, the delivery to the target cell is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.

Provided herein is a method of making a lipid particle, comprising a) providing a producer cell as any of those provided herein, b) culturing the cell under conditions that allow for production of the lipid particle, and c) separating, enriching, or purifying the lipid particle from the cell, thereby making the lipid particle. In some of any of the provided embodiments, the lipid particle is a pseudotyped lentiviral vector.

Provided herein is a lipid particle produced by any of the provided claims. Provided herein is a composition comprising a plurality of any of the provided lipid particles. In some of any of the provided embodiments, the composition further comprises a pharmaceutically acceptable carrier.

Provided herein is a method of transducing a cell comprising contacting a cell with any of the provided lipid particles or compositions. Provided herein is a method of delivering an exogenous agent into a target cell, the method comprising contacting a lipid particle provided herein or a composition provided herein with a target cell.

In some of any of the provided embodiments, the contacting is in vitro or ex vivo. In some of any of the provided embodiments, the contacting is in vivo in a subject.

Provided herein is a method of delivering an exogenous agent to a cell in a subject, the method comprising administering to the subject any of the provided lipid particles or compositions. In some of any of the provided embodiments, the exogenous agent is or encodes a therapeutic agent for treating a disease or condition in the subject.

Provided herein is a method of treatment, the method comprising administering to the subject any of the provided lipid particles or compositions. In some of any of the provided embodiments, the exogenous agent is or encodes a membrane protein, optionally a chimeric antigen receptor, for targeting an antigen associated with a disease or condition in the subject. In some of any of the provided embodiments, the exogenous agent is for use in gene therapy to correct a genetic deficiency or replaces a deficient or missing gene in the subject. In some of any of the provided embodiments, the subject is a human subject.

Provided herein is a co-display polypeptide, comprising one or more of: a leader domain; one or more fibronectin fragments; a hinge domain; an anchor domain; and a intracellular domain.

In some embodiments, the co-display polypeptide is partially derived or derived from one or more recombinant fibronectin fragments. In some embodiments, the one or more recombinant fibronectin fragments are selected from: CH296-A, CH296-B, CH296-C, CH296-D, CH296-E, and CH296-F.

Provided herein is a co-display polypeptide, comprising one or more of: a leader domain; a CS-1 domain; a linker; a C-domain; a hinge domain; an anchor domain; and a intracellular domain.

### BRIEF DESCRIPTION OF THE DRAWINGS

An exemplary pseudotyped vector is shown with an exemplary co-display protein in FIG. 1.

FIG. 2A depicts titer as sorted by co-display protein anchor domain. FIG. 2B depicts titer as sorted by co-display protein anchor domain.

Transduction of PanT cells is shown in FIG. 3A. Comparative activation of CD3 co-display in vector which also expresses a CD8 retargeted fusogen is shown in FIG. 3B. PanT transduction and activation for exemplary co-display constructs is shown in FIG. 3C.

FIG. 4A depicts GFP expression as a percentage of total cells as sorted by co-display protein anchor domain. FIG. 4B depicts GFP expression as sorted by co-display protein linker domain. Expression of CD8 retargeted NiV-G and CD3 co-display protein is shown in FIG. 4C.

FIG. 5A depicts GFP positive T cells following transduction with an exemplary co-display construct. Off-target transduction for the exemplary co-display construct is shown in FIG. 5B.

FIG. 6 depicts tumor cell (Nalm6) killing following transduction of a vector comprising a co-display construct, CD8 retargeted fusogen, as well as CAR transgene.

FIG. 7A depicts transduction with a retronectin co-display on BaEVTR LV particles. Transduction efficiency as a function of plasmid ratios is shown in FIG. 7B. Transduction efficiency across transduction dilutions is depicted in FIG. 7C. FIG. 7D shows BaEVTR fusogen and retronectin co-display assessed via infectivity on mobilized peripheral blood (mPB) CD34+ cells. CD34+ editing titers are shown in FIG. 7E. FIG. 7F shows a schematic of a retronectin co-display protein.

FIG. 8A shows a schematic of the modification introduced in the BaEVTR protease cleavage site.

FIG. 8B shows a schematic of a VLP comprising a BaEVTR (-MACA) protein and a RetroNectin Co-Display.

FIG. 8C shows percentage of GFP+ cells for a titration curves of BaEVTR with (+) or without (−) MACA and with (+) or without (−) Co-Display (CoD).

FIG. 8D shows experimental details used to test the indicated VLP in a short-term humanization model for 16 weeks.

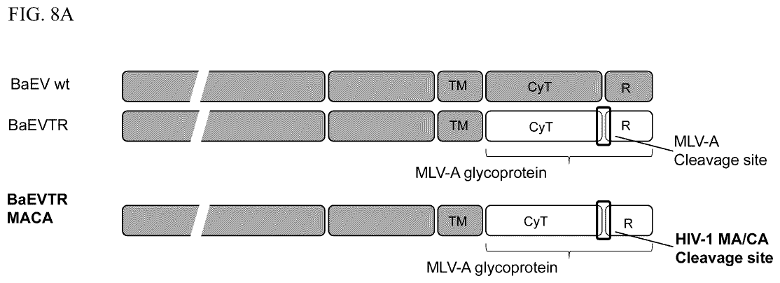

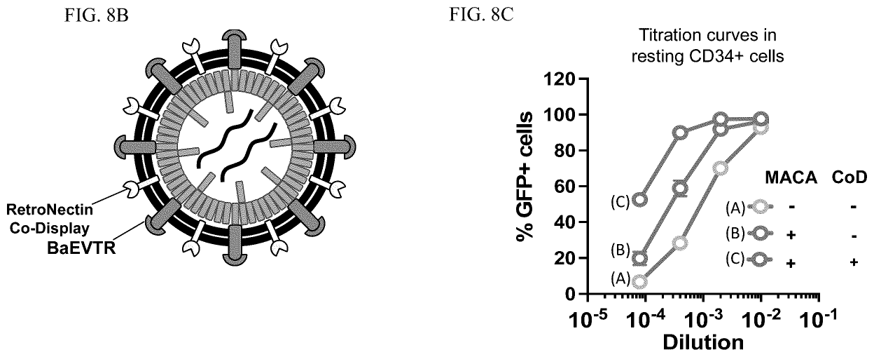

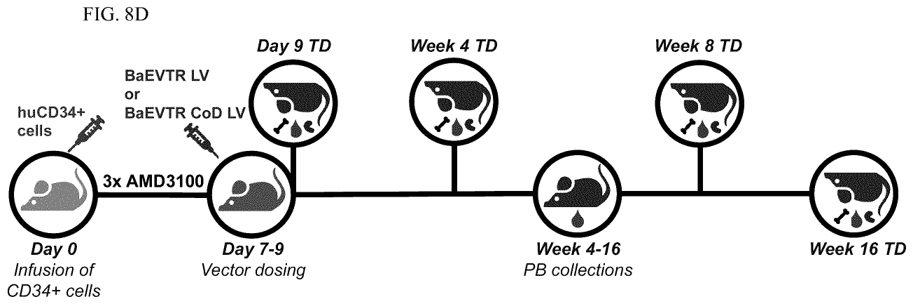

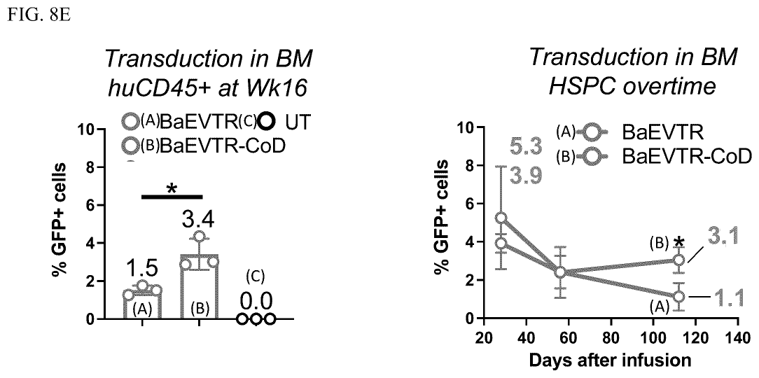

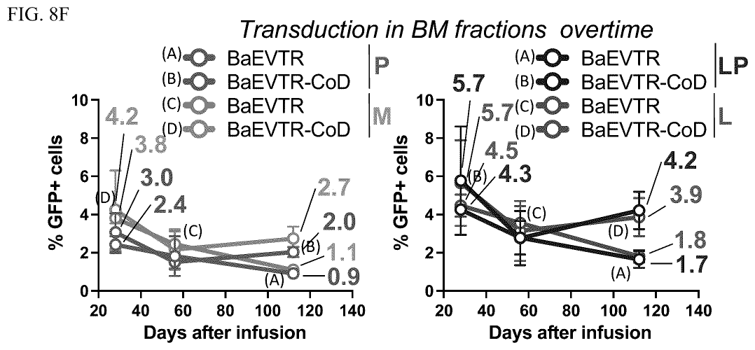

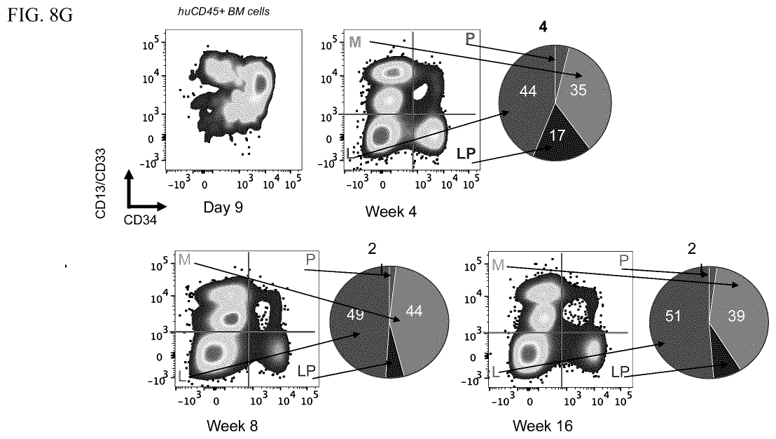

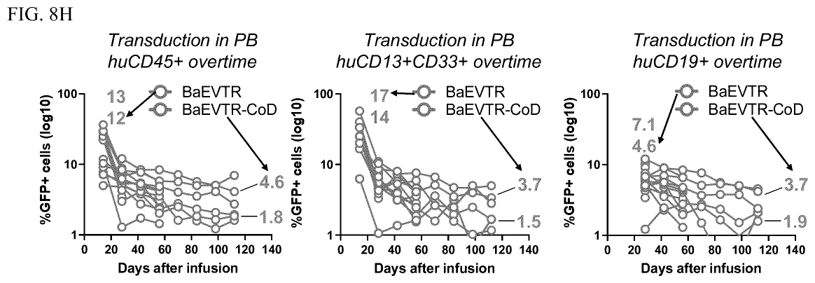

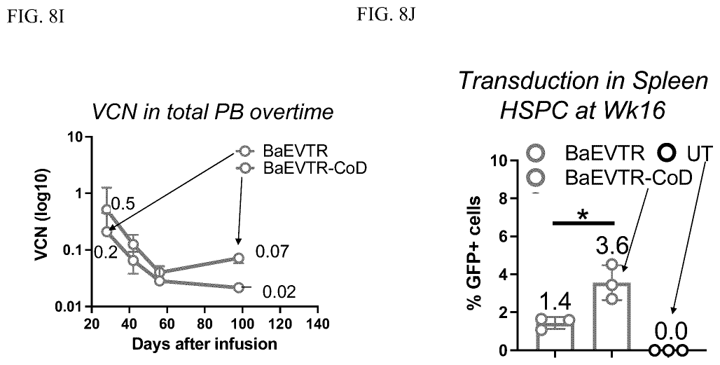

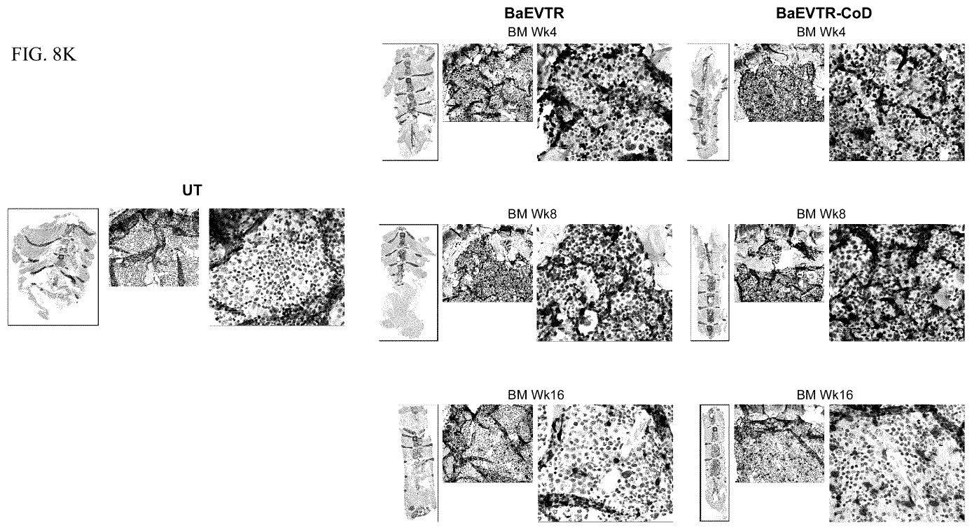

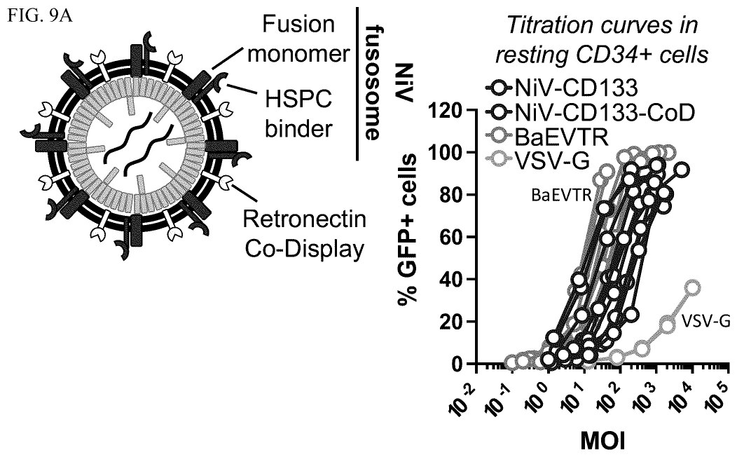

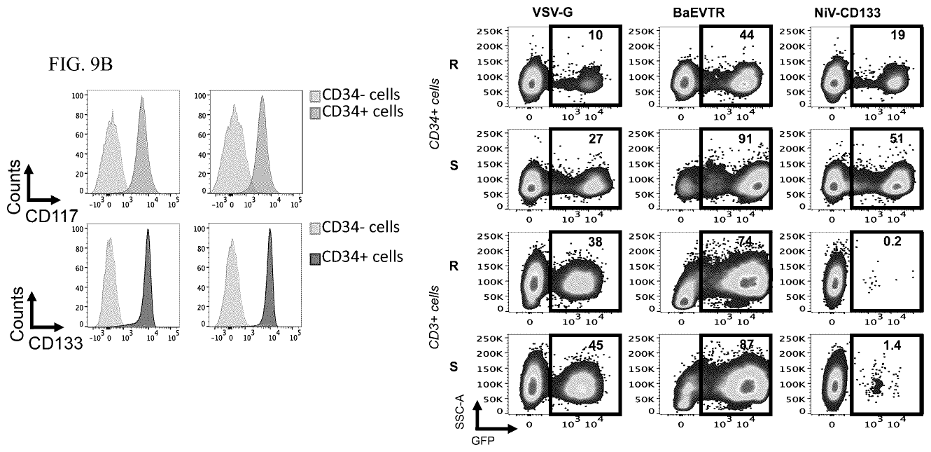

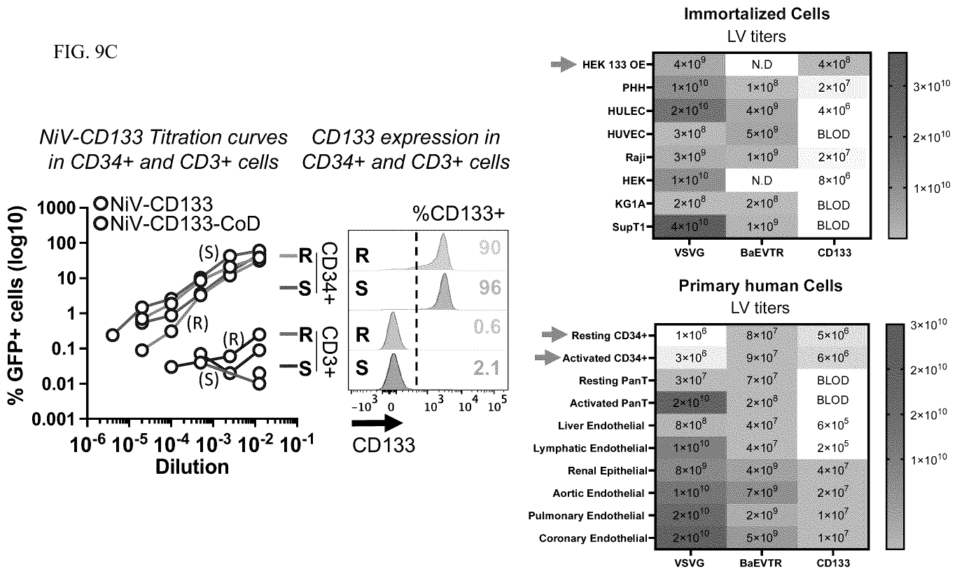

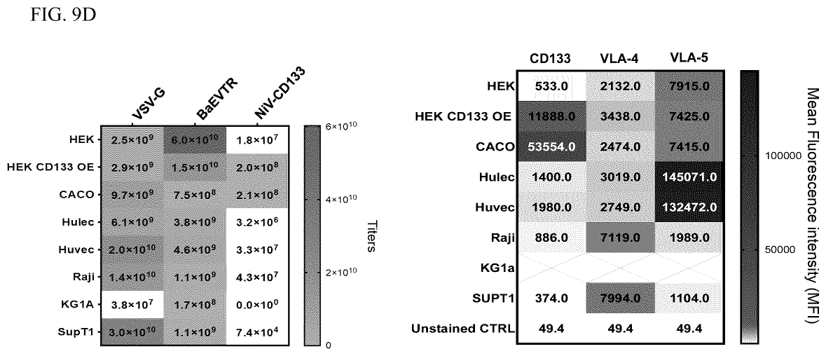

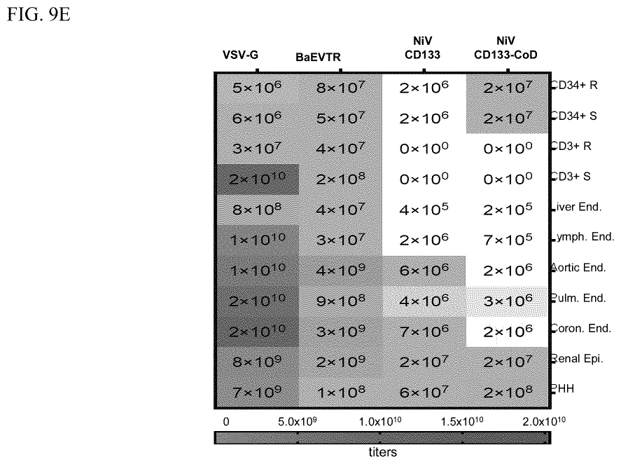

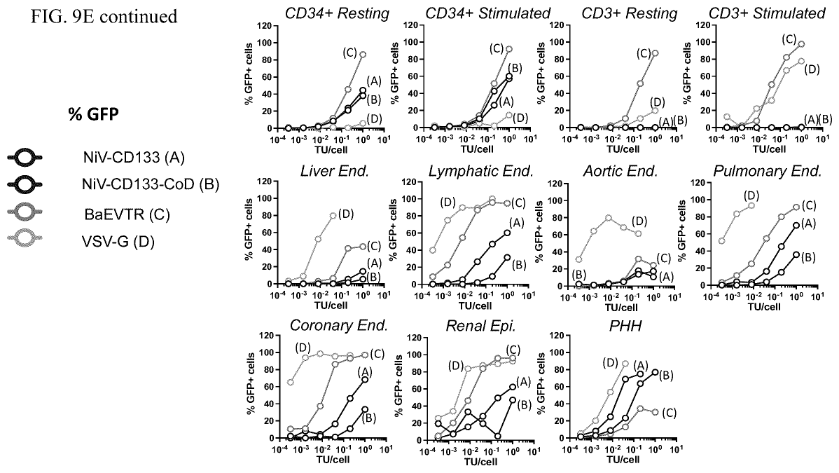

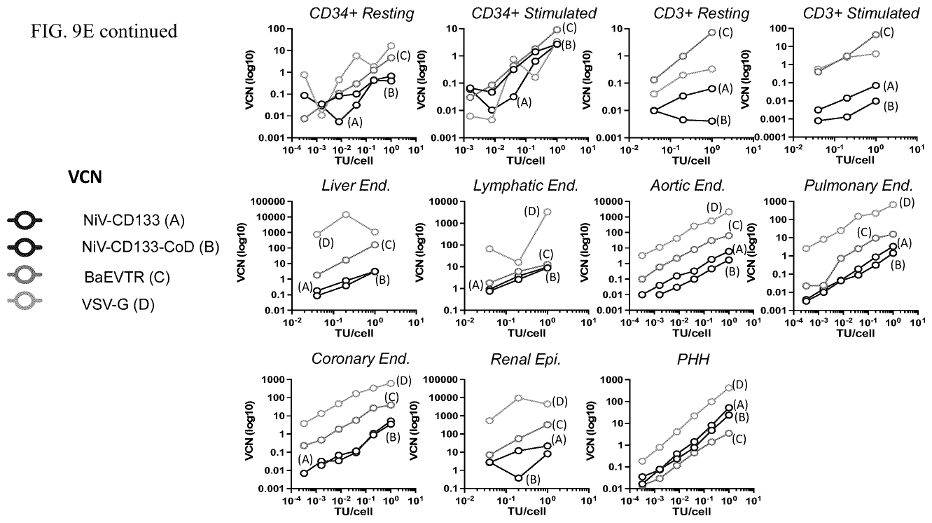

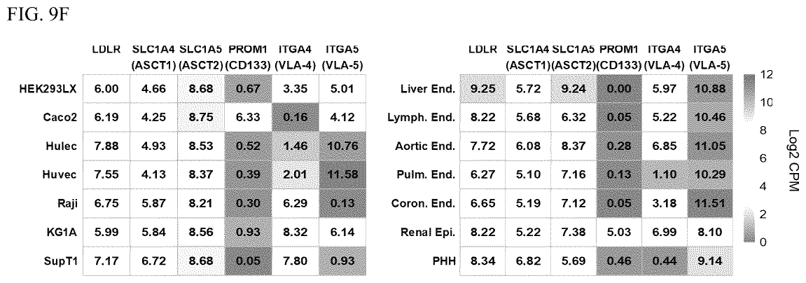

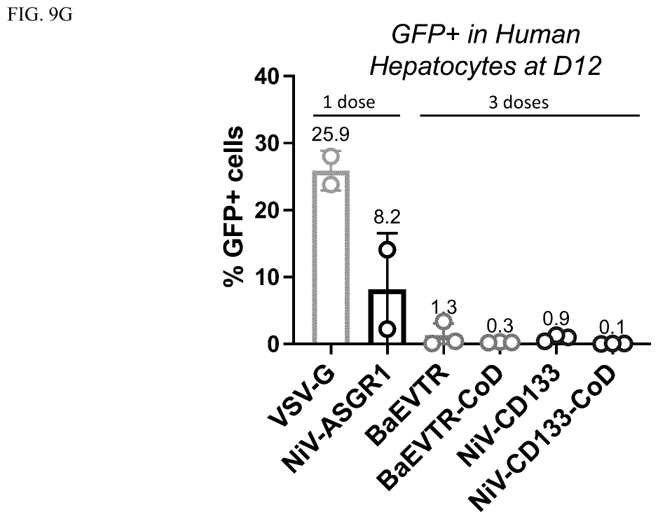

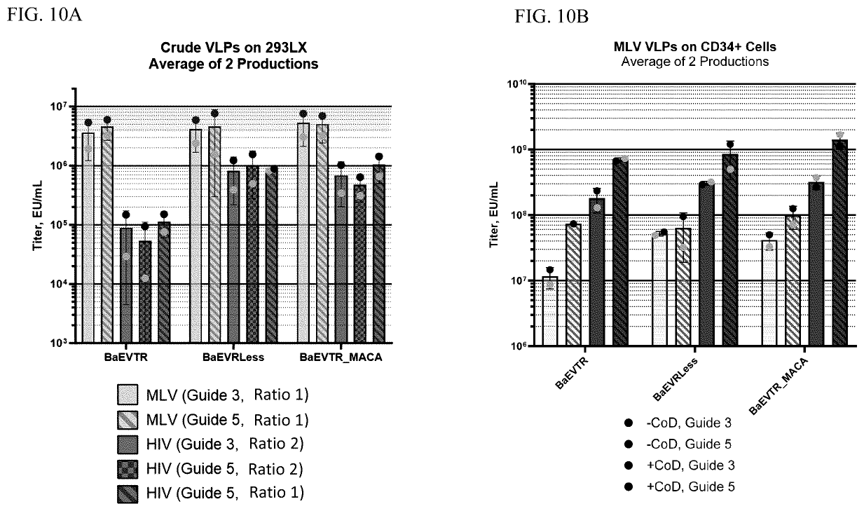

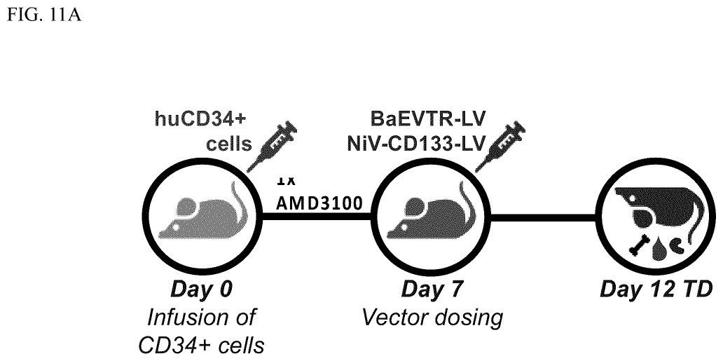

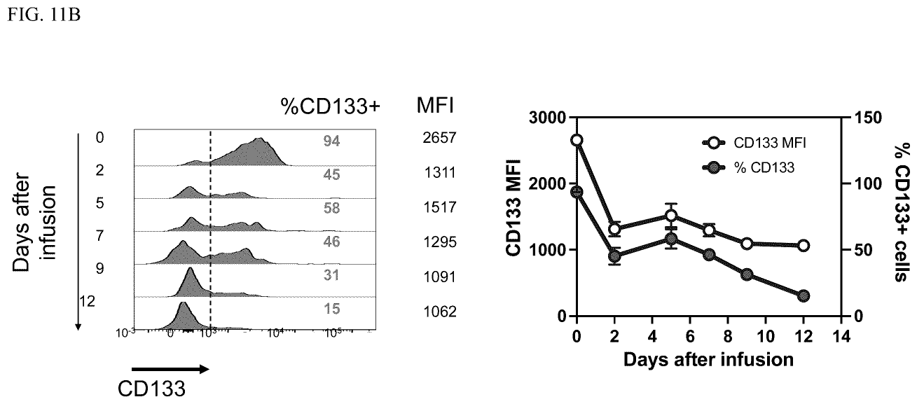

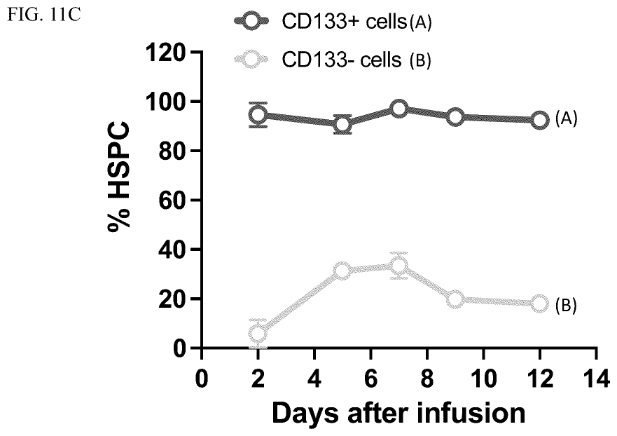

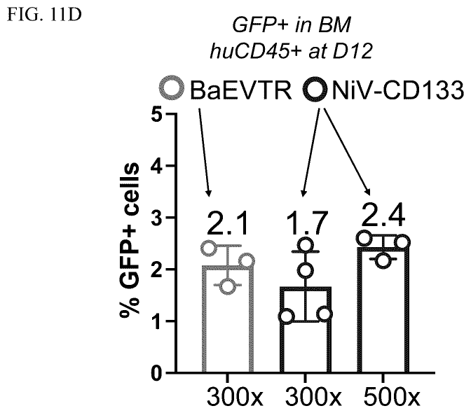

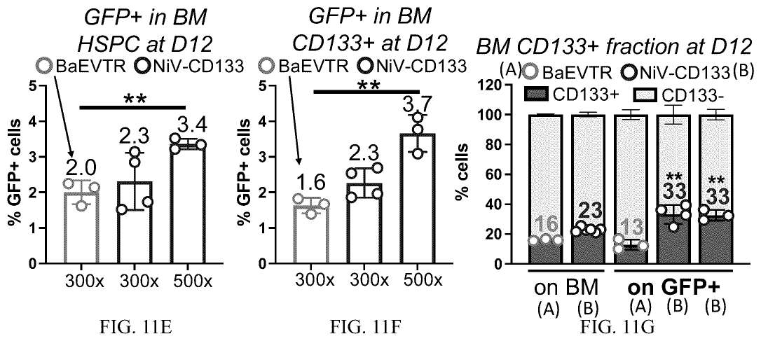

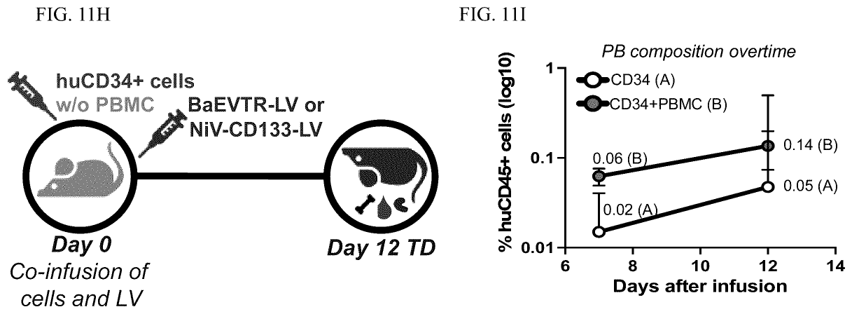

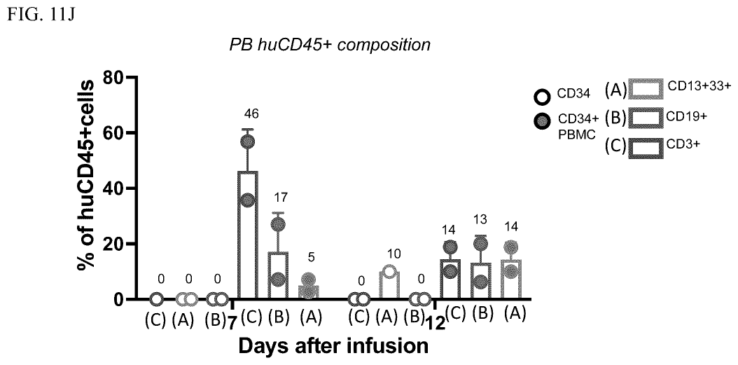

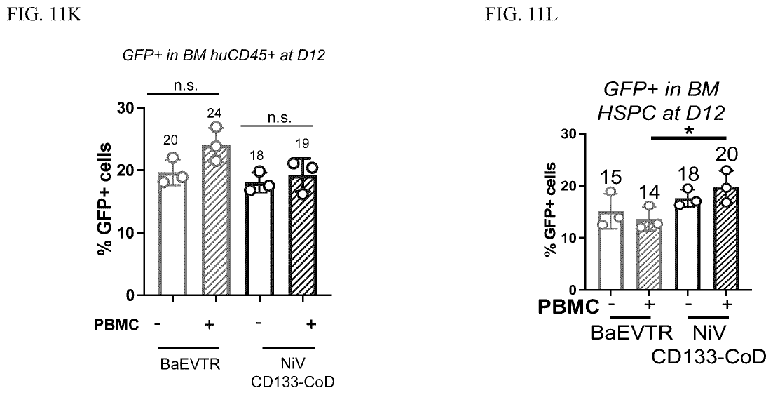

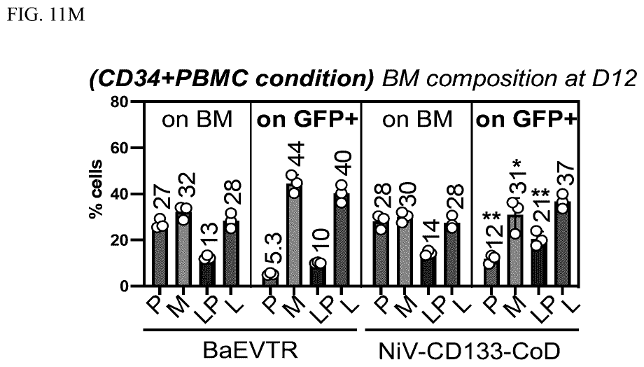

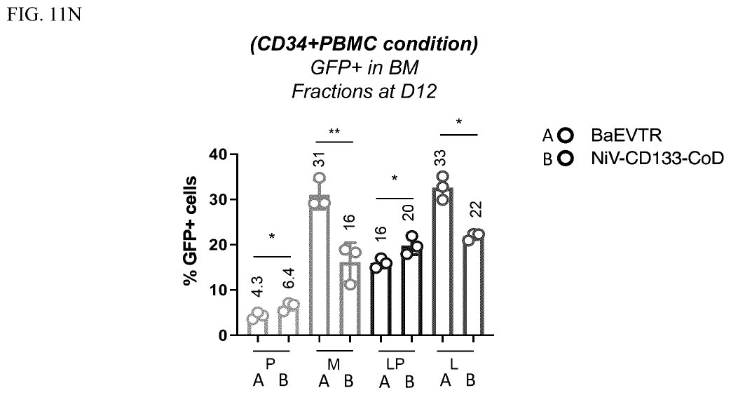

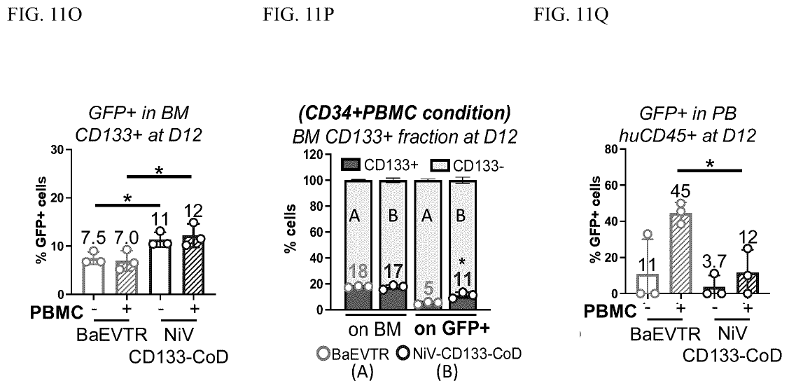

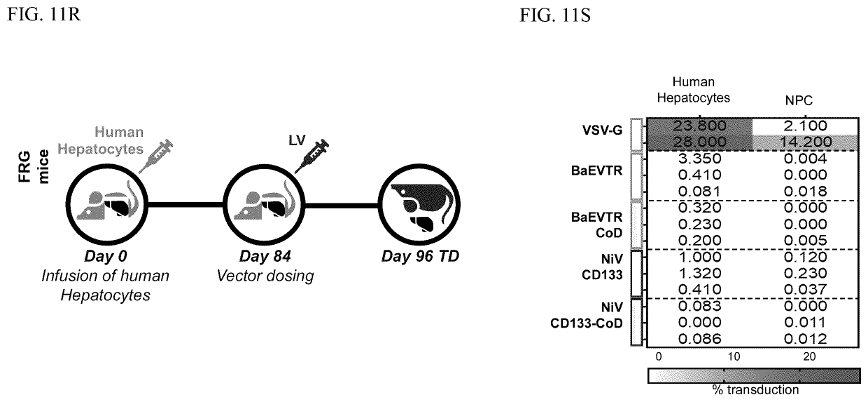

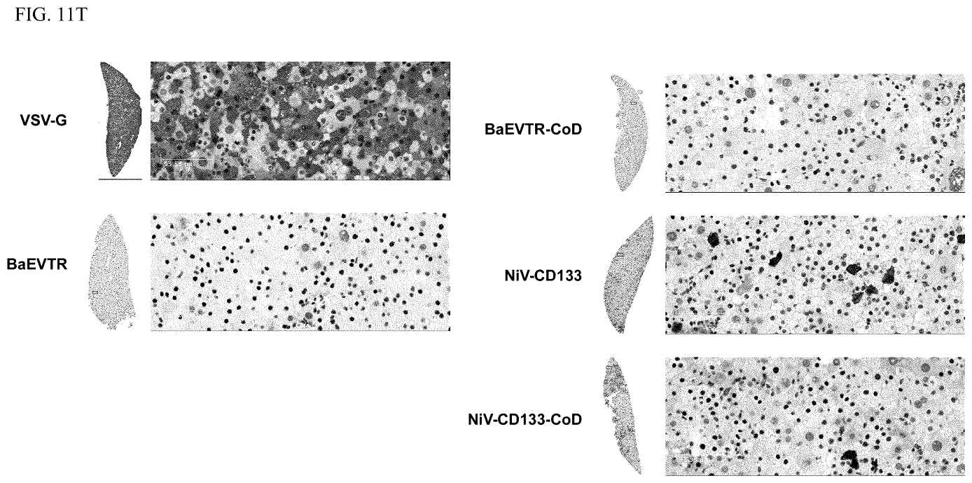

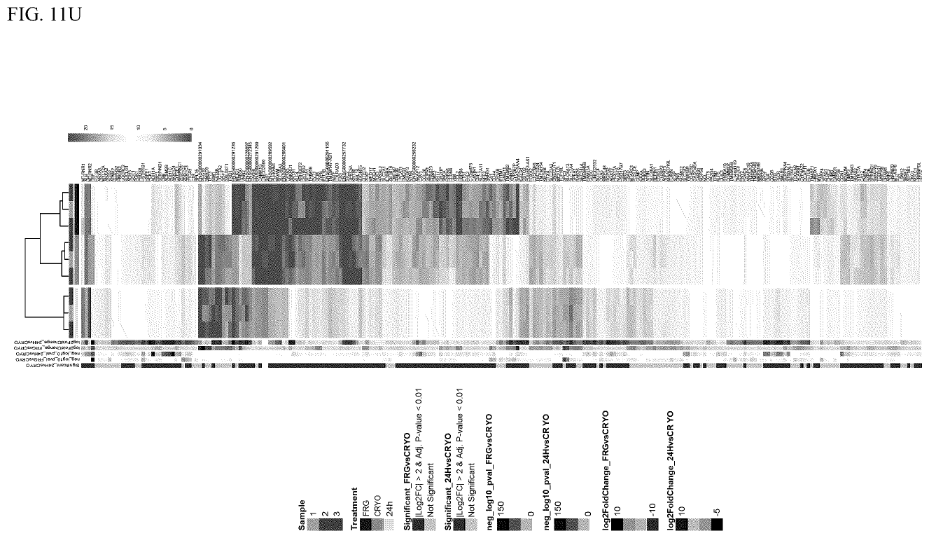

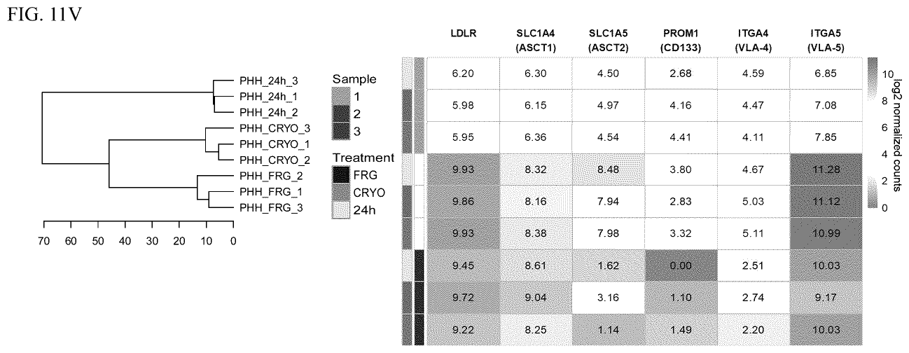

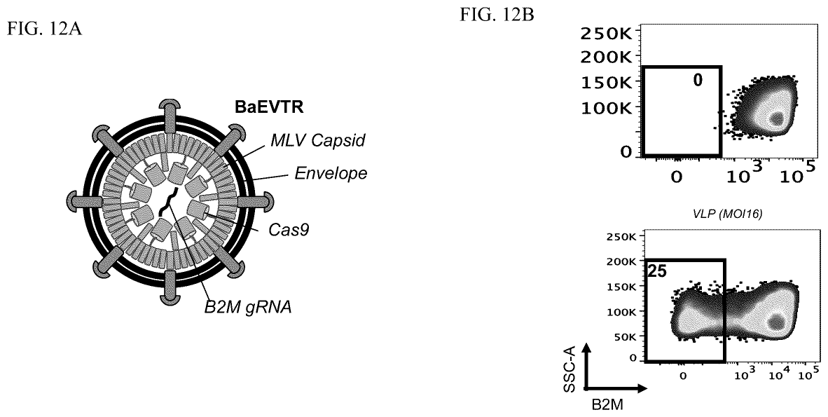

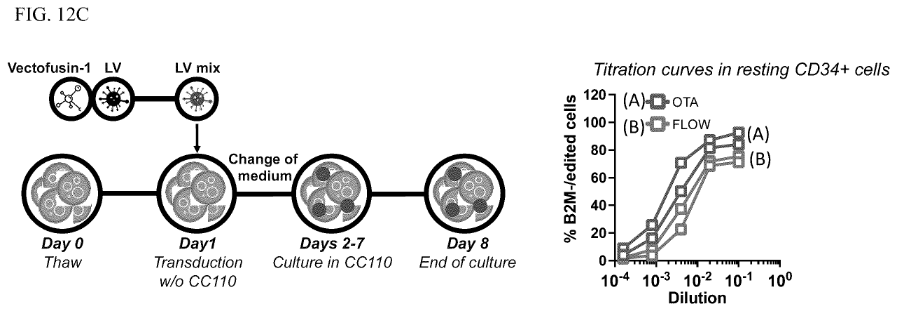

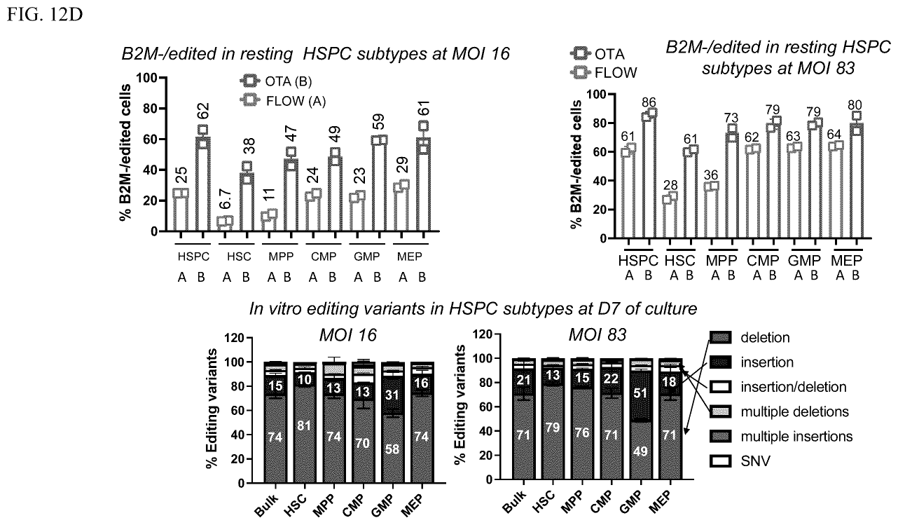

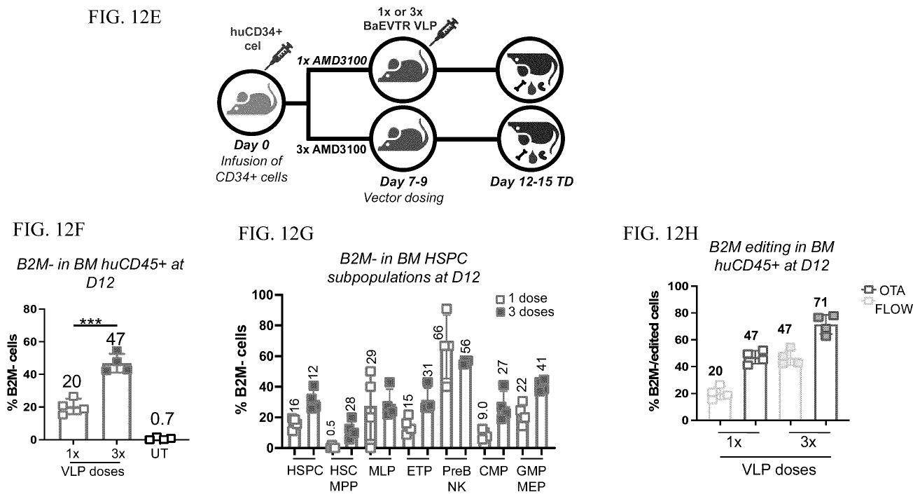

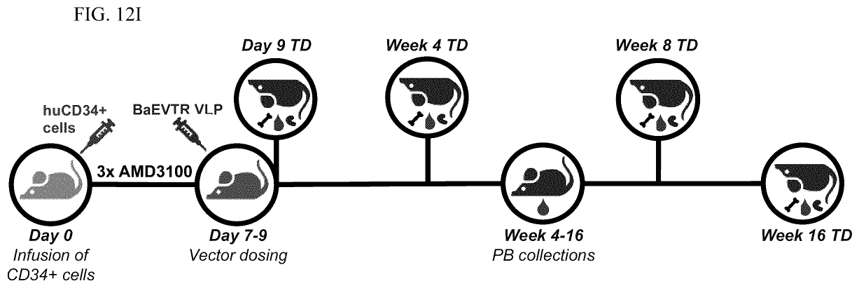

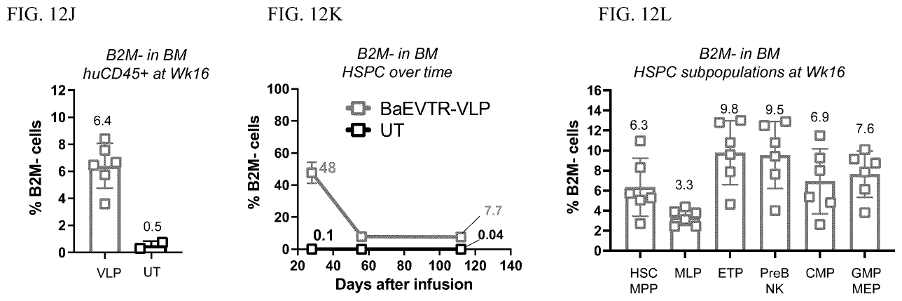

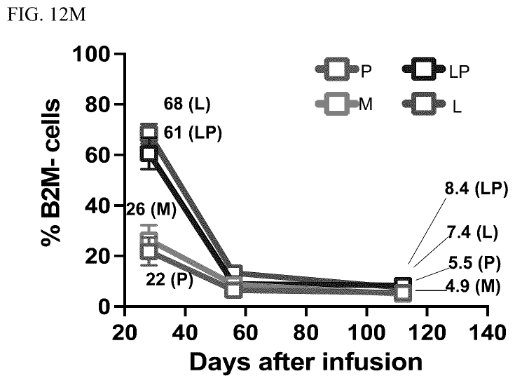

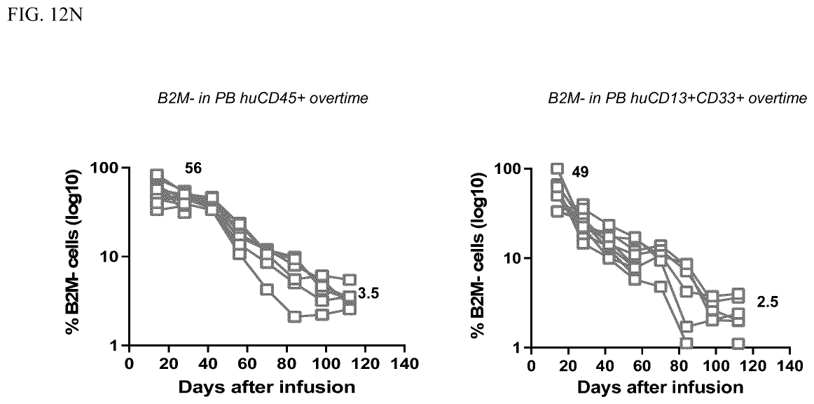

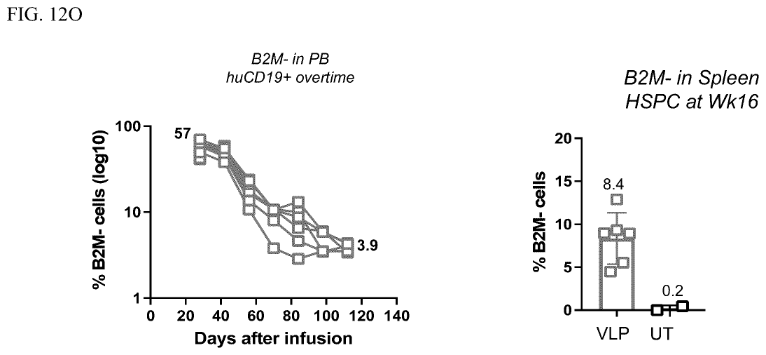

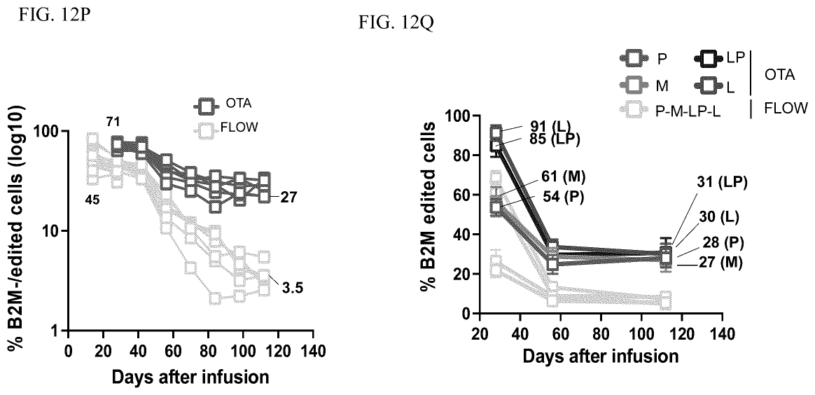

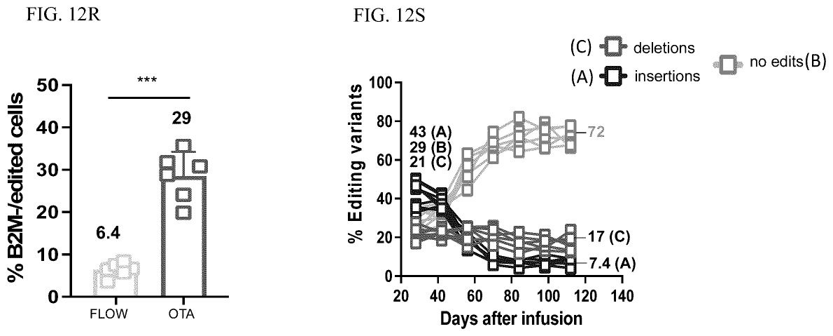

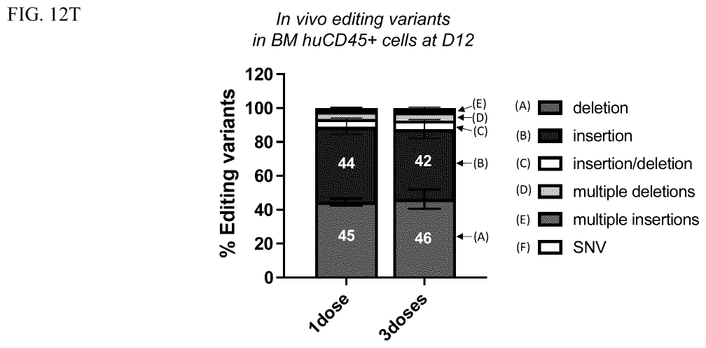

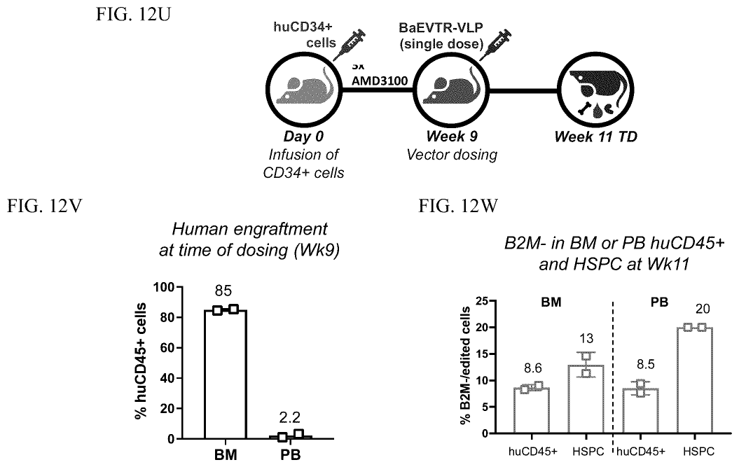

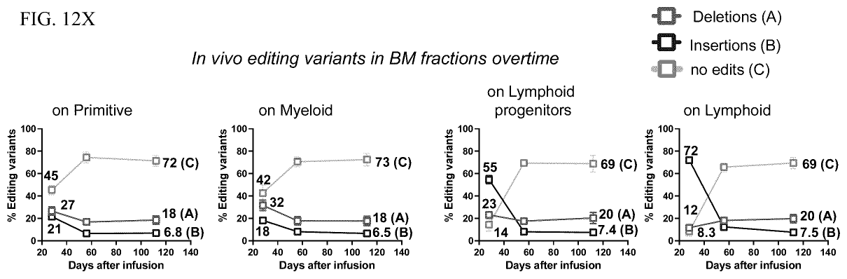

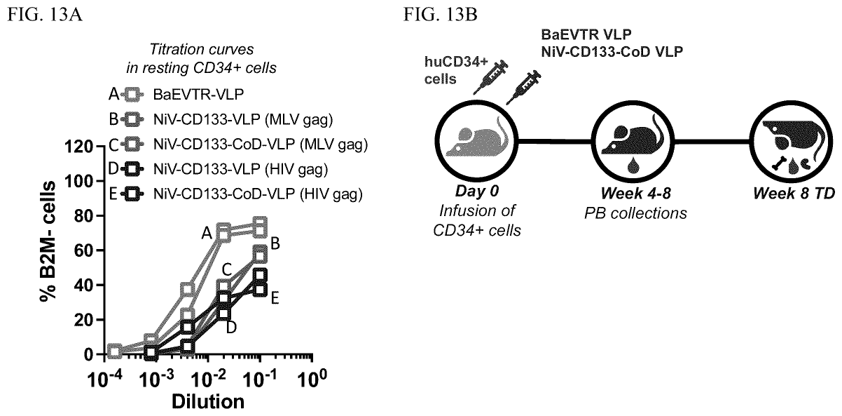

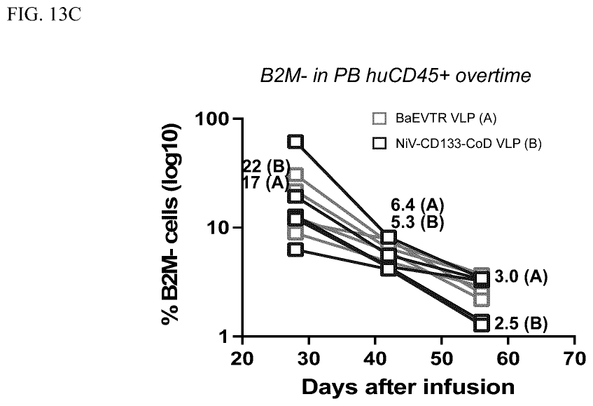

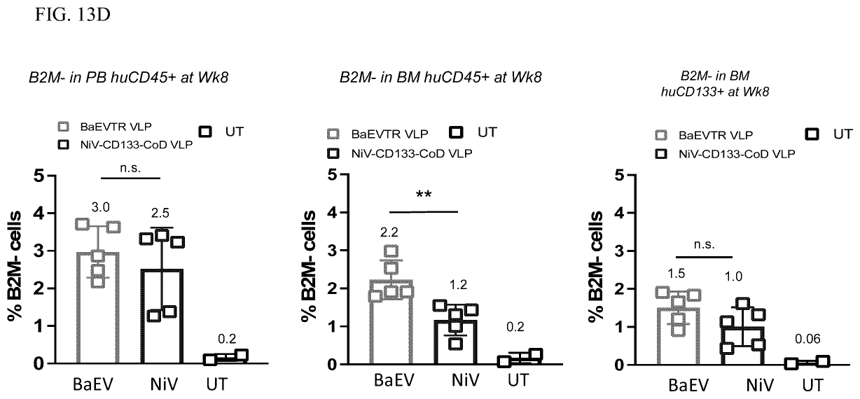

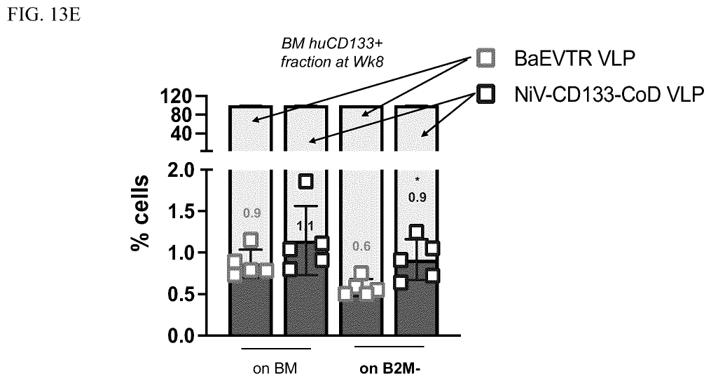

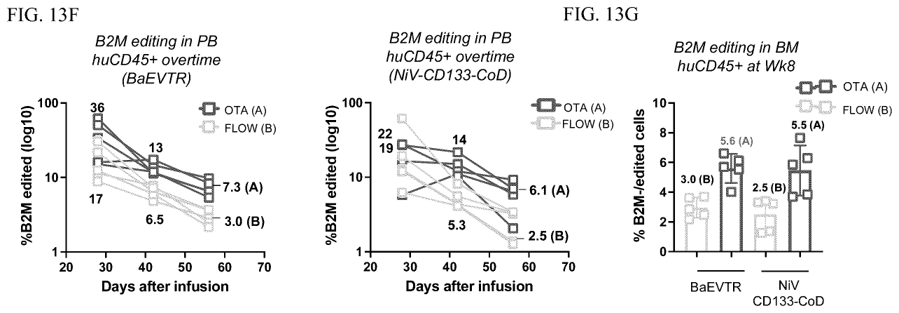

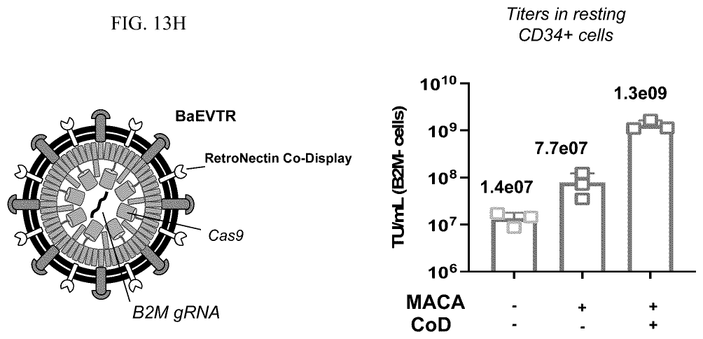

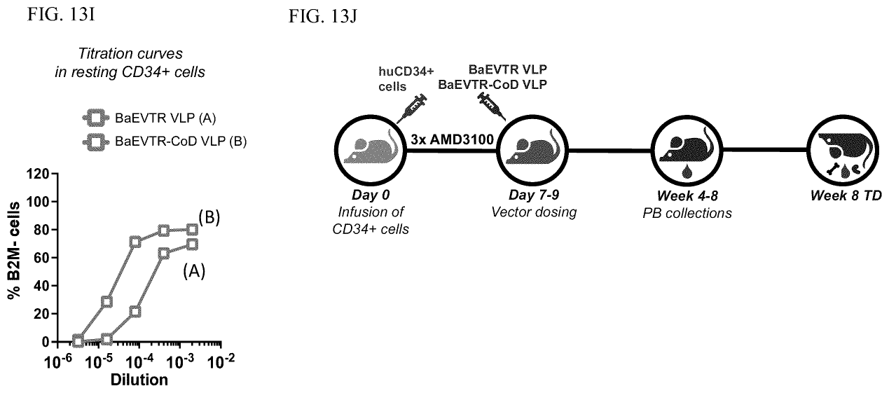

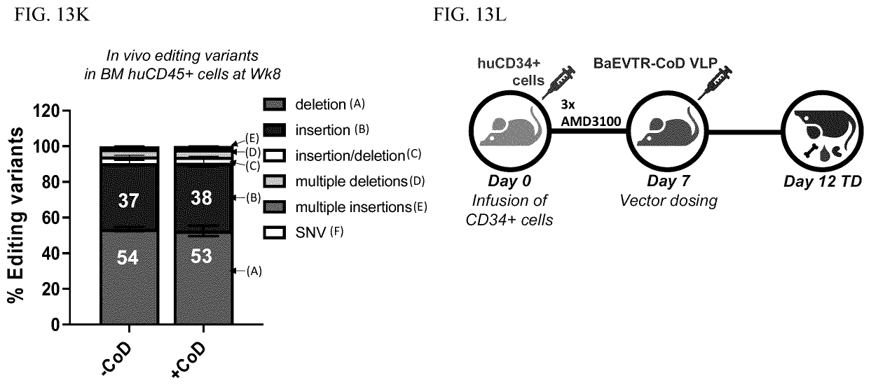

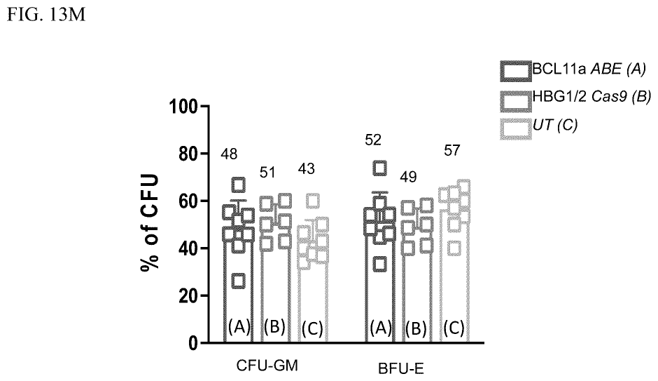

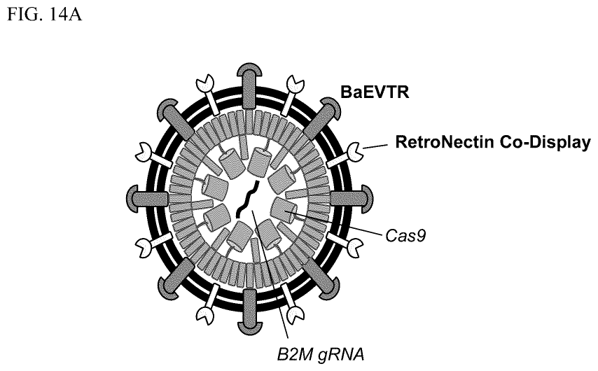

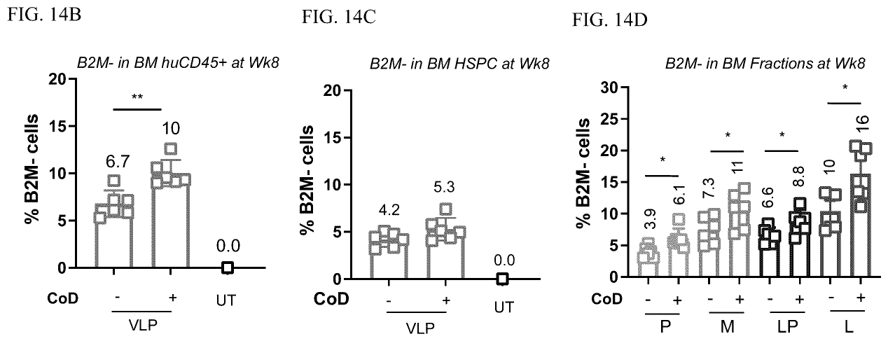

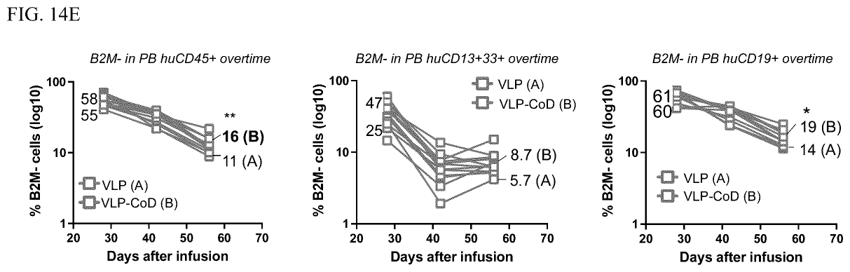

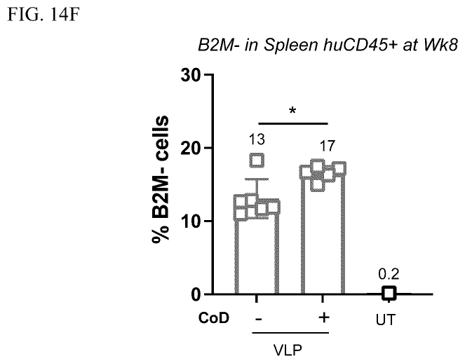

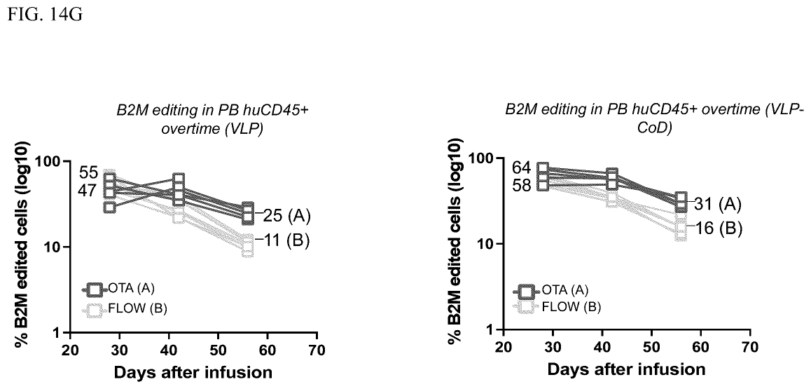

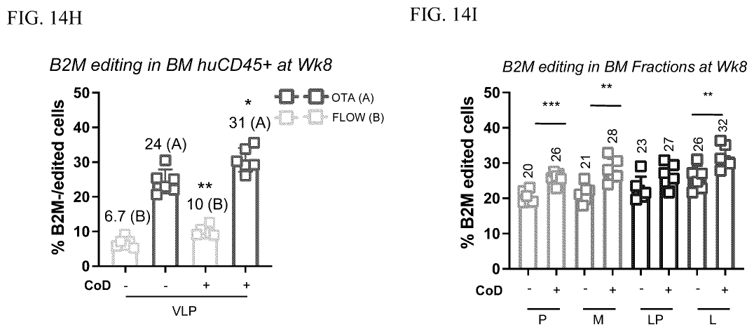

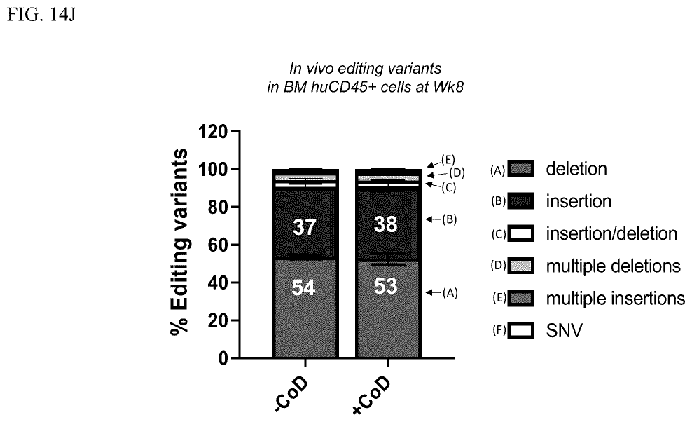

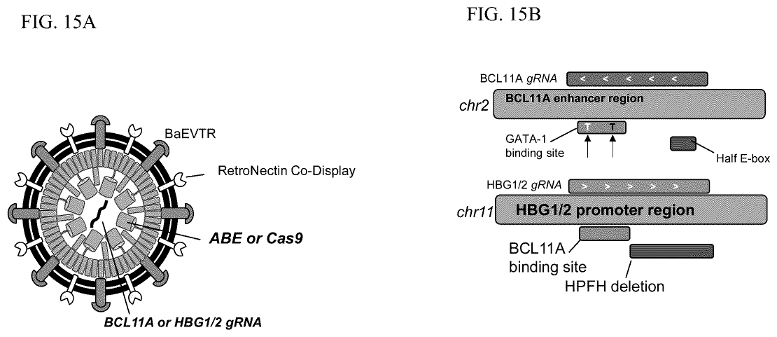

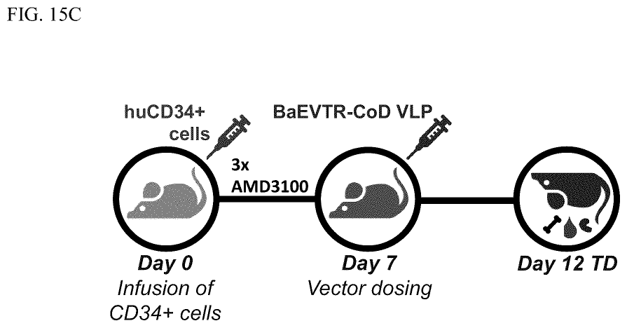

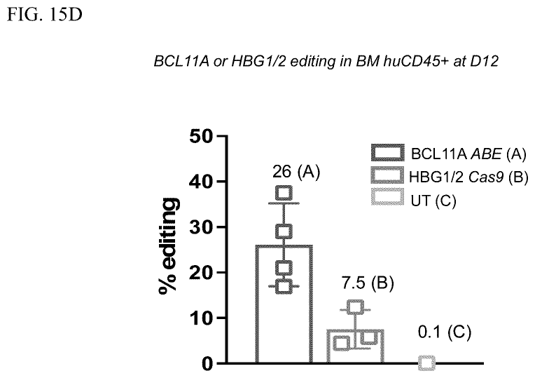

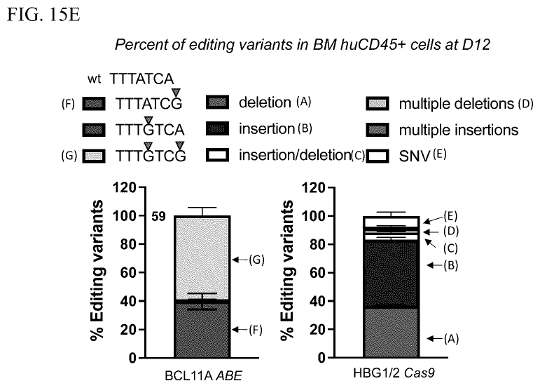

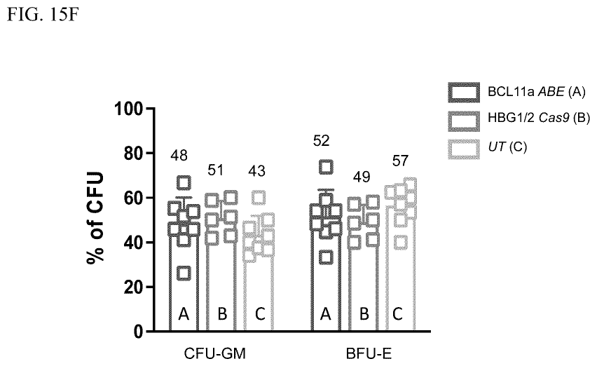

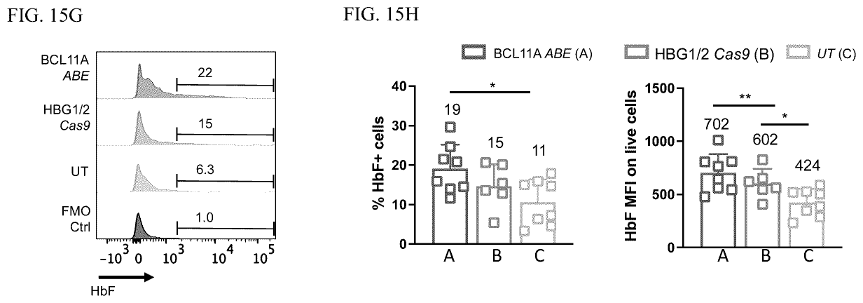

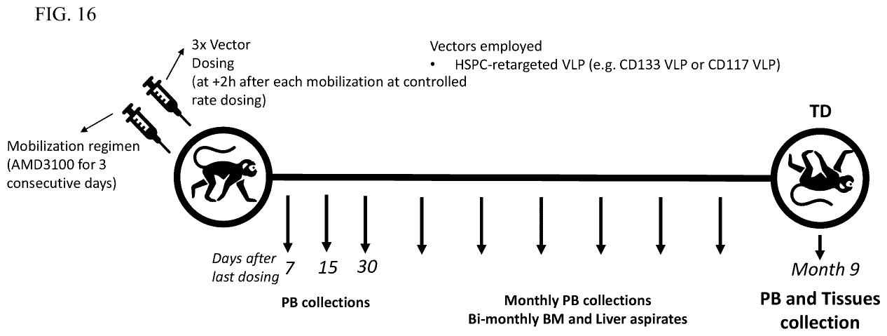

In vivo transduction efficiency of a BaEVTR co-display LV is shown in FIGS. 8E-8K. FIG. 8E shows percentage of GFP+ cells in bone marrow (BM) huCD45+ cells at week 16 for the indicated conditions. FIG. 8E shows percentage of GFP+ cells in BM HSPC over time for the indicated conditions. FIG. 8F shows percentage of GFP+ cells in BM fractions over time for the indicated conditions. M—myeloid, LP—lymphoid progenitor, L—lymphoid, and P—primitive. FIG. 8G shows flow plots for huCD45+ BM fractions in the short-term humanization model described in FIG. 8D. M—myeloid, LP—lymphoid progenitor. and L—lymphoid. FIG. 8H shows percentage GFP+ cells (log 10) in peripheral blood (PB) huCD45+ cells over time (left panel), PB huCD13+CD33+ cells over time (middle panel), and PB huCD19+ cells over time (right panel). FIG. 8I shows viral copy number (VCN) in total peripheral blood (PB) over time for the indicated conditions. FIG. 8J shows percentage GFP+ cells in spleen HSPC at week 16 for the indicated conditions. FIG. 8K shows histology performed on sternums collected from treated animals at different timepoints after infusion.

Assessment of NiV LV-CD133, with or without retronectin co-display, for transduction of resting CD34+ cells is shown in FIG. 9A. Left panel shows a schematic of an exemplary VLP. Right panel shows titration curves for the indicated VLPs in resting CD34+ cells.

FIG. 9B shows CD117 and CD133 expression in CD34 negative (−) and CD34 positive (+) cells. FIG. 9B shows transduction efficiency in resting and stimulated CD34+ cells.

FIG. 9C depicts a full titration curve with exemplary target cell titer heat map.

FIG. 9D shows titers as a heatmap for the various combinations of VLP and cell type.

FIG. 9E shows titers as heatmaps for the various combinations of VLP and cell type (left panel, flow cytometry plots of percentage GFP+ cells for the indicated VLPs (right panel), and plots of VCNs for the indicated VLPs (9F continued).

FIG. 9F shows heatmaps of expression levels for the targets in the various cell types.

Transduction of human hepatocytes in vivo is shown in FIG. 9G.

FIG. 10A. shows comparative titer of BaEV fusogen pseudotyped VLP on recipient 293LX cells. FIG. 10B. shows comparative titer of BaEV fusogen pseudotyped VLP on recipient CD34+ cells.

FIG. 11A shows experimental details for the D7 post-humanization model used to test the indicated VLP (or LV).

FIG. 11B shows CD133 expression in the infused human CD34+ cells (day 0) and CD133 expression over time.

FIG. 11C percentage of HPSC cells that are CD133 negative (−) or CD133 positive (+) over time.

FIG. 11D-G shows transduction of human CD45+ cells using pseudotyped lentiviral vector. FIG. 11D shows percentage of GFP positive (GFP+) cells in bone marrow (BM) huCD45+ cells at day 12 (D12) for the indicated VLPs. FIG. 11E shows percentage of GFP positive (GFP+) cells in bone marrow (BM) HSPC at day 12 (D12) for the indicated VLPs. FIG. 11F shows percentage of GFP positive (GFP+) cells in bone marrow (BM) CD133+ cells at day 12 (D12) for the indicated VLPs. FIG. 11G shows the percentage of cells that are BM CD133+ cells for each of the indicated subpopulations.

FIG. 11H shows experimental details for co-infusion model used to test the indicated VLP (or LV).

FIG. 11I shows percentage of huCD45+ cells (log 10) in peripheral blood (PB) over time.

FIG. 11J shows percentage of huCD45+ cells for each of the indicated groups.

FIG. 11K shows percentage of huCD45+ cells that are GFP+ for each of the indicated groups.

BaEVTR LV and NiV-CD133-CoD-LV transduction efficiency in the presence of off-target cellular sinks is shown in FIG. 11L-Q. FIG. 11L shows percentage of GFP+ cells in bone marrow (BM) HSPC at day 12 (D12) for the indicated group. FIG. 11M and FIG. 11N shows percentage of cells of each BM fraction in the BM composition at day 12 (D12) for the indicated groups. FIG. 11O shows percentage of GFP+ cells in BM CD133+ cells at D12. FIG. 11P shows percentage of cells in BM CD133+ fraction at day 12. FIG. 11Q shows percentage of GFP+ cells in peripheral blood (PB) huCD45+ cells at D12.

FIG. 11R shows experimental details for a model used to test the VLP (or LV).

FIG. 11S shows a heatmap summarizing the transduction for each of the reagents in human hepatocytes or mouse non-parenchymal cells (NPC).

FIG. 11T shows histology of liver for livers isolated from mice having received the indicated treatment.

FIG. 11U shows a heatmap of RNA-seq data comparing freshly thawed PHH, PHH cultured in vitro for 24 h (time of vector dosing for the in vitro titration experiments) and human hepatocytes freshly isolated from FRG mice prior to vector dosing.

FIG. 11V left panel shows hierarchical clustering of the RNA-seq data from FIG. 11U. FIG. 11V right panel shows a heatmap of RNA-seq data for a selection of genes from FIG. 11U.

FIG. 12A shows a schematic of a VLP having a BaEVTR envelope and carrying a Beta 2 Microglobulin (B2M) guide RNA and a CRISPR-Cas9 editor covalently fused with a MLV capsid.

FIG. 12B shows a representative flow strategy for readout of gene editing of the B2M locus.

FIG. 12C (left panel) shows an experimental strategy for in vitro testing of gene editing efficiency of a BaEVTR VLP. FIG. 12C (right panel) shows titration curves as measured by OTA and flow for BaEVTR tested using the strategy shown in the left panel.

FIG. 12D shows gene editing efficiency in 5 HSPC FACS-sorted fractions. Upper left panel shows gene editing efficiency in 5 HSPC FACS-sorted fractions following transduction with BaEVTR VLP at an MOI of 16. Upper right panel shows gene editing efficiency in 5 HSPC FACS-sorted fractions following transduction with BaEVTR VLP at an MOI of 83. Lower panel shows the relative proportion of editing variants in each HSPC subtype as well as in the bulk HSPC population following transduction with BaEVTR VLP at an MOI 16 or 83.

FIG. 12E shows an experimental strategy for in vivo testing using a D7 post-humanization model.

FIG. 12F shows results of gene editing efficiency as measured by flow cytometry for the indications conditions in BM huCD45+ at day 12 (D12).

FIG. 12G shows editing efficiency as measured by flow cytometry for individual HSPC subtypes in BM at day 12 (D12).

FIG. 12H shows editing efficiency as measured by flow cytometry and OTA for BM huCD45+ at day 12 (D12).

FIG. 12I shows a schematic of a long-term follow up study was conducted, similarly to what done for testing the LV constructs, with interim BM takedowns at week 4 and week 8 and terminal readout at week 16 after 3 VLP dosing at D7-D9 post humanization.

FIG. 12J shows percentage of BM2 negative cells as measured by flow cytometry in BM huCD45+ cells at week 16.

FIG. 12K shows percentage of B2M negative cells as measured by flow cytometry in BM HSPC over time.

FIG. 12L shows percentage of B2M negative cells as measured by flow cytometry in BM HSCP subpopulations at week 16.

FIG. 12M shows percentage of B2M negative cells in four human BM factions over time.

FIG. 12N shows percentage of B2M negative cells in huCD45+, huCD13+CD33+, and huCD19+ populations over time.

FIG. 12O shows percentage of B2M edited cells in spleen HSPC at week 16.

FIG. 12P shows percentage of B2M edited cells over time as measured by flow cytometry and OTA.

FIG. 12Q shows percentage of B2M edited cells over time as measured by flow cytometry and OTA.

FIG. 12R shows percentage of B2M edited cells as in multipotent P fraction as measured by flow cytometry and OTA.

FIG. 12S shows percentage of editing variants (no edits, insertions, and deletions) in PB over time.

FIG. 12T shows the relative proportion of editing variants for BM huCD45+ at day 12 (D12) for the indicated conditions.

FIG. 12U shows an experimental strategy for testing VLP in a limiting scenario with a high number of human target cells inject at 9 weeks post-humanization.

FIG. 12V shows percentage of huCD45+ cells at week 9 in the experimental model described in FIG. 12U.

FIG. 12W shows percentage of B2M edited cells in huCD45+ bone marrow (BM) or peripheral blood (PB) at week 11.

FIG. 12X shows relative percentage of editing variants in BM fractions over time: primitive, myeloid, lymphoid progenitors, and on lymphoid.

FIG. 13A shows percentage of B2M edited cells in resting CD34+ cells for the indicated VLPs at each of the indicated dilution series.

FIG. 13B shows an experimental strategy for testing VLPs in a co-infusion model.

FIG. 13C shows percentage of B2M edited cells (log 10) in PB huCD45+ cells over time up to week 8 after cell and vector infusion.

FIG. 13D shows percentage of B2M edited cells in peripheral blood (PB), bone marrow (BM) and huCD133+ BM cells at week 8 for the indicated VLPs.

FIG. 13E shows percentage of B2M edited cells for the indicated VLPs when gated on huCD133+ cells.

FIG. 13F shows percentage of B2M edited cells (log 10) in peripheral blood (PB) huCD45+ over time after being treated with BaEVTR VLP (left panel) or NiV-CD133-CoD (right panel).

FIG. 13G shows percentage of B2M edited cells using OTA and flow cytometry in bone marrow (BM) huCD45+ cells for the indicated VLPs at week 8.

FIG. 13H left panel shows an exemplary VLP. FIG. 13H right panel shows titers (TU/mL (B2M− cells) in resting CD34+ cells for BaEVTR with (+) or without (−) MACA and with (+) or without (−) CoDisplay (CoD).

FIG. 13I shows percentage of B2M edited cells in resting CD34+ cells at the indicated titrations for BaEVTR VLP and BaEVTR-CoD VLP.

FIG. 13J shows an experimental strategy for testing VLPs in D7 post-humanization model with PB collections at weeks 4-8 and takedown (TD) at week 8.

FIG. 13K shows in vivo editing variants in huCD45+ cells at week 8. FIG. 13L shows an experimental strategy for testing VLPs in D7 post-humanization model and takedown (TD) at day 12.

FIG. 13M shows % of colony forming units (CFU) for each of the indicated treatment conditions.

FIG. 14A shows a schematic of a VLP having a BaEVTR envelope, a RetroNectin Co-Display and carrying a Beta 2 Microglobulin (B2M) guide RNA and a CRISPR-Cas9 editor covalently fused with a MLV capsid.

FIG. 14B shows percentage of B2M negative cells in bone marrow (BM) huCD45+ cells at week 8 for BaEVTR without (−) and with (+) Codisplay (CoD).

FIG. 14C shows percentage of B2M negative cells in bone marrow (BM) HSPC cells at week 8 for BaEVTR without (−) and with (+) Codisplay (CoD).

FIG. 14D shows percentage of B2M negative cells in bone marrow (BM) fractions: P—primitive; M—myeloid; LP—lymphoid progenitors; and L—lymphoid.

FIG. 14E shows percentage of B2M negative cells over time in PB huCD45+(left panel), PB huCD13+CD33+(middle panel); PB huCD19+ cells (right panel) for the indicated VLPs.

FIG. 14F shows percentage of B2M negative cells in Spleen huCD45+ cells at week 8 for the indicated VLPs.

FIG. 14G shows percentage of B2M edited cells (log 10) in peripheral blood (PB) huCD45+ cells over time as measured by OTA and flow for BaEVTR without CoDisplay (VLP) and with CoDisplay (VLP-CoD).

FIG. 14H shows percentage of B2M edited cells (log 10) in bone marrow (BM) huCD45+ cells at week 8 as measured by OTA and flow for BaEVTR without CoDisplay (VLP) and with CoDisplay (VLP-CoD).

FIG. 14I shows percentage of B2M edited cells (log 10) in bone marrow (BM) fractions at week 8 as measured by OTA and flow for BaEVTR without CoDisplay (VLP) and with CoDisplay (VLP-CoD).

FIG. 14J shows relative percentage of editing variants in the B2M locus with respect to what observed with the BaEVTR VLP design.

FIG. 15A shows a schematic of a VLP having a BaEVTR envelope, a RetroNectin Co-Display and carrying a Beta 2 Microglobulin (B2M) guide RNA and a CRISPR-Cas9 editor or a Adenine Base Editor (ABE) covalently fused with a MLV capsid.

FIG. 15B shows a schematic for guide RNA targeting an enhancer region of Bcl11a or a HBG1/2 promoter region.

FIG. 15C shows experimental details for testing for the indicated VLPs.

FIG. 15D shows percentage of editing at the Bcl11a locus or a HBG1/2 locus in bone marrow (BM) huCD45+ cell at day 12 (D12).

FIG. 15E shows relative percentage of editing variants at the Bcl11 locus edited with the ABE or at the HBG1/2 locus with Cas9.

FIG. 15F show number of CFU-GM and BFU-E colonies for each condition tested.

FIG. 15G shows flow cytometry plots of HbF expression following editing with the indicated conditions.

FIG. 15H shows percentage of HbF positive cells (left panel) and HbF MFI on live cells for the indicated conditions.

FIG. 16 shows a model study design for non-human primates.

### I. DETAILED DESCRIPTION

Provided herein is a lipid particle, such as a lentiviral vector or viral vector particle, that contains at least one viral envelope attachment protein, such as derived from paramyxovirus or baboon endogenous virus (BaEV). The viral envelope attachment proteins are designed to provide for targeted delivery of the lipid particles to a target cells. In some embodiments, the lipid particles also contain a codisplay protein, sch as a transduction adjuvant or immune stimulating protein.

Among provided lipid particles are those in which the viral attachment protein is derived from a paramyxovirus and is a retargeted attachment protein comprising (i) a paramyxovirus envelope attachment protein; and (ii) a targeting moiety directed to a target molecule expressed on the surface of a target cell. In such embodiments, the lipid particles also contain a fusion (F) protein that mediates fusion of the lipid particle with the target cell. In some embodiments, the paramyxovirus attachment protein, fusion protein, and codisplay protein are exposed on the outside surface of the lipid bilayer. In some embodiments, the paramyxovirus attachment protein, fusion protein, and codisplay protein are embedded in the lipid bilayer.

Also among provided lipid particles are those in which the viral attachment protein is derived from baboon endogenous virus (BaEV). In some embodiments, the BaEV is a variant of wild-type BaEV that contains one or more mutations that promote cleavage of a region of the protein to remove the inhibitory domain, thereby improving expression and function and targeted delivery of lipid particles containing the variant BaEV. In some embodiments, the BaEV protein, such as a variant BaEV protein, and codisplay protein are exposed on the outside surface of the lipid bilayer. In some embodiments, the BaEV protein, such as a variant BaEV protein, and codisplay protein are embedded in the lipid bilayer.

In some embodiments, the lipid particles can be a virus-like particle, a virus, or a viral vector, such as a lentiviral vector.

The provided lipid particles, such as lentiviral vectors, exhibit advantages over available envelope-pseudotyped particles. For instance, VSV-G is the most common envelope glycoprotein used for pseudotyping but its broad tropism is often not ideal or desirable for specific target cell delivery, such as is desired for gene therapy or exogenous protein delivery. Further, although alternative envelope proteins may exhibit reduced tropism or may be amenable to linkage to a binding domain for redirected targeting to a desired target cell, the titer of a preparation of lentiviral vectors containing such envelope proteins may be too low to allow for efficient transduction. Thus, alternative approaches are needed. It is found herein that certain duplicate proteins when pseudotyped on a lentiviral vector exhibit high titers.

The provided lipid particles, such as lentiviral vectors, exhibit advantages over available envelope-pseudotyped particles produced without a co-display protein. For instance, delivery efficiency and potency of viral vectors are often not ideal or desirable for specific target cell delivery, such as is desired for gene therapy or exogenous protein delivery. Further, titer of a preparation of lentiviral vectors containing such co-display protein may be significantly increased with respect to efficient transduction. Thus, alternative approaches are needed. It is found herein that certain co-display proteins when expressed with a fusogen pseudotyped on a lentiviral vector exhibit high titers.

Also provided are lipid particles, such as targeted lipid particles, additionally containing one or more exogenous agents, such as for delivery of a diagnostic or therapeutic agent to cells, including following in vivo administration to a subject. Also provided herein are methods and uses of the lipid particles, such in diagnostic and therapeutic methods. Also provided are polynucleotides, methods for engineering, preparing, and producing the lipid non-cell particles, compositions containing the particles, and kits and devices containing and for using, producing and administering the particles.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### II. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, “lipid particle” refers to any biological or synthetic particle that contains a bilayer of amphipathic lipids enclosing a lumen or cavity. Typically a lipid particle does not contain a nucleus. Such lipid particles include, but are not limited to, viral particles (e.g. lentiviral particles), virus-like particles, viral vectors (e.g., lentiviral vectors) exosomes, enucleated cells, various vesicles, such as a microvesicle, a membrane vesicle, an extracellular membrane vesicle, a plasma membrane vesicle, a giant plasma membrane vesicle, an apoptotic body, a mitoparticle, a pyrenocyte, or a lysosome. In some embodiments, a lipid particle can be a fusosome. In some embodiments, the lipid particle is not a platelet. In some embodiments, the fusosome is derived from a source cell. A lipid particle also may include an exogenous agent or a nucleic acid encoding an exogenous agent, which may be present in the lumen of the lipid particle.

The terms “viral vector particle” and “viral vector” are used interchangeably herein and refer to a vector for transfer of an exogenous agent (e.g. non-viral or exogenous nucleic acid) into a recipient or target cell and that contains one or more viral structural proteins in addition to at least one non-structural viral genomic component or functional fragment thereof (i.e., a polymerase, an integrase, a protease or other non-structural component). The viral vector thus contains the exogenous agent, such as heterologous nucleic acid that includes non-viral coding sequences, to be transferred into a cell. Examples of viral vectors are retroviral vectors, such as lentiviral vectors.

The term “retroviral vector” refers to a viral vector that contains retroviral nucleic acid or is derived from a retrovirus. A retroviral vector particle includes the following components: a vector genome (retrovirus nucleic acid), a nucleocapsid encapsidating the nucleic acid, and a membrane envelope surrounding the nucleocapsid. Typically, a retroviral vector contains sufficient retroviral genetic information to allow packaging of an RNA genome, in the presence of packaging components, into a viral particle capable of infecting a target cell. Infection of the target cell may include reverse transcription and integration into the target cell genome. A retroviral vector may be a recombinant retroviral vector that is replication defective and lacks genes essential for replication, such as a functional gag-pol and/or env gene and/or other genes essential for replication. A retroviral vector also may be a self-inactivating (SIN) vector.

As used herein, a “lentiviral vector” or LV refers to a viral vector that contains lentiviral nucleic acid or is derived from a lentivirus. A lentiviral vector particle includes the following components: a vector genome (lentivirus nucleic acid), a nucleocapsid encapsidating the nucleic acid, and a membrane surrounding the nucleocapsid. Typically, a lentiviral vector contains sufficient lentiviral genetic information to allow packaging of an RNA genome, in the presence of packaging components, into a viral particle capable of infecting a target cell. Infection of the target cell may include reverse transcription and integration into the target cell genome. A lentiviral vector may be a recombinant lentiviral vector that is replication defective and lacks genes essential for replication, such as a functional gag-pol and/or env gene and/or other genes essential for replication. A lentiviral vector also may be a self-inactivating (SIN) vector.

As used herein, a “retroviral nucleic acid,” refers to a nucleic acid containing at least the minimal sequence requirements for packaging into a retroviral vector, alone or in combination with a helper cell, helper virus, or helper plasmid. In the case of “lentiviral nucleic acid” the nucleic acid refers to at least the minimal sequence requirements for packaging into a lentiviral vector, alone or in combination with a helper cell, helper virus, or helper plasmid. In some embodiments, the viral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi (Ψ)), RRE (e.g., to bind to Rev and promote nuclear export). The viral nucleic acid can comprise RNA (e.g., when part of a virion) or DNA (e.g., when being introduced into a source cell or after reverse transcription in a recipient cell). In some embodiments, the viral nucleic acid is packaged using a helper cell, helper virus, or helper plasmid which comprises one or more of (e.g., all of) gag, pol, and env.

As used herein, “fusosome” refers to a lipid particle containing a bilayer of amphipathic lipids enclosing a lumen or cavity and a fusogen that interacts with the amphipathic lipid bilayer. In some embodiments, the fusosome is a membrane enclosed preparation. In some embodiments, the fusosome is derived from a source cell. A fusosome also may include an exogenous agent or a nucleic acid encoding an exogenous agent, which may be present in the lumen of the fusosome.

As used herein, “fusosome composition” refers to a composition comprising one or more fusosomes.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., a lumen of a retroviral vector and a cytoplasm of a target cell). In some embodiments, the fusogen comprises a complex of two or more proteins, e.g., wherein neither protein has fusogenic activity alone. In some embodiments, the fusogen comprises a targeting domain. Examples of fusogens include paramyxovirus F and G proteins such as those from Nipah Virus (NiV) and biologically active portions or variants thereof including any as described.

As used herein, a “re-targeted fusogen,” such as a re-targeted G protein, refers to a fusogen that comprises a targeting moiety having a sequence that is not part of the naturally-occurring form of the fusogen in which the targeting moiety targets or binds a molecule on a desired cell type. In embodiments, the fusogen comprises a different targeting moiety relative to the targeting moiety in the naturally-occurring form of the fusogen. In embodiments, the naturally-occurring form of the fusogen lacks a targeting domain, and the re-targeted fusogen comprises a targeting moiety that is absent from the naturally-occurring form of the fusogen. In embodiments, the fusogen is modified to comprise a targeting moiety. In some such embodiments, the attachment of the targeting moiety to a fusogen (e.g. G protein) may be directly or indirectly via a linker, such as a peptide linker. In embodiments, the fusogen comprises one or more sequence alterations outside of the targeting moiety relative to the naturally-occurring form of the fusogen, e.g., in a transmembrane domain, fusogenically active domain, or cytoplasmic domain.

As used herein, a “target cell” refers to a cell of a type to which it is desired that a lipid particle, such as a targeted lipid particle, delivers an exogenous agent. In embodiments, a target cell is a cell of a specific tissue type or class, e.g., an immune effector cell, e.g., a T cell. In some embodiments, a target cell is a diseased cell, e.g., a cancer cell. In some embodiments, the fusogen, e.g., re-targeted fusogen leads to preferential delivery of the exogenous agent to a target cell compared to a non-target cell.

As used herein a “non-target cell” refers to a cell of a type to which it is not desired that a lipid particle delivers an exogenous agent. In some embodiments, a non-target cell is a cell of a specific tissue type or class. In some embodiments, a non-target cell is a non-diseased cell, e.g., a non-cancerous cell. In some embodiments, the fusogen, e.g., re-targeted fusogen leads to lower delivery of the exogenous agent to a non-target cell compared to a target cell.

As used herein a “biologically active portion,” such as with reference to a protein such as a G protein or an F protein, refers to a portion of the protein that exhibits or retains an activity or property of the full-length of the protein. For example, a biologically active portion of an F protein retains fusogenic activity in conjunction with the G protein when each are embedded in a lipid bilayer. A biologically active portion of the G protein retains fusogenic activity in conjunction with an F protein when each is embedded in a lipid bilayer. The retained activity can include 10%-150% or more of the activity of a full-length or wild-type F protein or G protein. Examples of biologically active portions of F and G proteins include proteins with truncations of the cytoplasmic domain, such as any of the described variant NiV-F with a truncated cytoplasmic tail.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved binding.

TABLE 1
Exemplary substitutions

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The term “effective amount” as used herein means an amount of a pharmaceutical composition which is sufficient to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “exogenous agent” as used herein with reference to a lipid particle, such as a viral vector, refers to an agent that is neither comprised by nor encoded in the corresponding wild-type virus or fusosome made from a corresponding wild-type source cell. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises RNA or protein.

As used herein, a “promoter” refers to a cis-regulatory DNA sequence that, when operably linked to a gene coding sequence, drives transcription of the gene. The promoter may comprise a transcription factor binding sites. In some embodiments, a promoter works in concert with one or more enhancers which are distal to the gene.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

A “disease” or “disorder” as used herein refers to a condition where treatment is needed and/or desired.

As used herein, the terms “treat,” “treating,” or “treatment” refer to ameliorating a disease or disorder, e.g., slowing or arresting or reducing the development of the disease or disorder or reducing at least one of the clinical symptoms thereof. For purposes of this disclosure, ameliorating a disease or disorder can include obtaining a beneficial or desired clinical result that includes, but is not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

The terms “individual” and “subject” are used interchangeably herein to refer to an animal; for example a mammal. The term patient includes human and veterinary subjects. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. The subject can be male or female and can be any suitable age, including infant, juvenile, adolescent, adult, and geriatric subjects. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder. In particular embodiments, the subject is a human, such as a human patient.

### III. LIPID PARTICLES COMPRISING FUSOGEN

In some embodiments, the lipid particles contain one or more fusogens. In some embodiments, the lipid particle contains an exogenous or overexpressed fusogen. In some embodiments, the fusogen is disposed in the lipid bilayer. In some embodiments, the fusogen facilitates the fusion of the lipid particle (e.g., viral vector) to a membrane. In some embodiments, the membrane is a plasma cell membrane of a target cell. In some embodiments, the lipid particle (e.g., viral vector) comprising the fusogen integrates into the membrane into a lipid bilayer of a target cell. In some embodiments, the fusogen results in mixing between lipids in the lipid particle (e.g., viral vector) and lipids in the target cell. In some embodiments, the fusogen results in formation of one or more pores between the interior of the non-cell particle and the cytosol of the target cell.

In some embodiments, fusogens are protein based, lipid based, and chemical based fusogens. In some embodiments, the lipid particle (e.g., viral vector), contain a first fusogen that is a protein fusogen and a second fusogen that is a lipid fusogen or chemical fusogen. In some embodiments, the fusogen binds a fusogen binding partner on a target cell surface. In some embodiments, the lipid particle (e.g., viral vector) is pseudotyped with the fusogen. In some examples, a virus of viral-like particle has a modification to one or more of its envelope proteins, e.g., an envelope protein is substituted with an envelope protein from another virus. In some embodiments, retroviral envelope proteins, e.g. lentiviral envelope proteins, are pseudotyped with a fusogen.

In some embodiments, the fusogen is a protein fusogen, e.g., a mammalian protein or a homologue of a mammalian protein (e.g., having 50%, 60%, 70%, 80%, 85%, 90%, 95%, 96%, 97%, 98%, 99%, or greater identity), a non-mammalian protein such as a viral protein or a homologue of a viral protein (e.g., having 50%, 60%, 70%, 80%, 85%, 90%, 95%, 96%, 97%, 98%, 99%, or greater identity), a native protein or a derivative of a native protein, a synthetic protein, a fragment thereof, a variant thereof, a protein fusion comprising one or more of the fusogens or fragments, and any combination thereof.

In some embodiments, the fusogen may include a mammalian protein. Examples of mammalian fusogens may include, but are not limited to, a SNARE family protein such as vSNAREs and tSNAREs, a syncytin protein such as Syncytin-1 (DOI: 10.1128/JVI.76.13.6442-6452.2002), and Syncytin-2, myomaker (biorxiv.org/content/early/2017/04/02/123158, doi.org/10.1101/123158, doi: 10.1096/fj.201600945R, doi:10.1038/nature12343), myomixer (www.nature.com/nature/journal/v499/n7458/full/nature12343.html, doi:10.1038/nature12343), myomerger (science.sciencemag.org/content/early/2017/04/05/science.aam9361, DOI: 10.1126/science.aam9361), FGFRL1 (fibroblast growth factor receptor-like 1), Minion (doi.org/10.1101/122697), an isoform of glyceraldehyde-3-phosphate dehydrogenase (GAPDH) (e.g., as disclosed in U.S. Pat. No. 6,099,857A), a gap junction protein such as connexin 43, connexin 40, connexin 45, connexin 32 or connexin 37 (e.g., as disclosed in US 2007/0224176, Hap2, any protein capable of inducing syncytium formation between heterologous cells, any protein with fusogen properties, a homologue thereof, a fragment thereof, a variant thereof, and a protein fusion comprising one or more proteins or fragments thereof. In some embodiments, the fusogen is encoded by a human endogenous retroviral element (hERV) found in the human genome. Additional exemplary fusogens are disclosed in U.S. Pat. No. 6,099,857A and US 2007/0224176, the entire contents of which are hereby incorporated by reference.

In some embodiments, the fusogen may include a non-mammalian protein, e.g., a viral protein. In some embodiments, a viral fusogen is a Class I viral membrane fusion protein, a Class II viral membrane protein, a Class III viral membrane fusion protein, a viral membrane glycoprotein, or other viral fusion proteins, or a homologue thereof, a fragment thereof, a variant thereof, or a protein fusion comprising one or more proteins or fragments thereof.

In some embodiments, Class I viral membrane fusion proteins include, but are not limited to, Baculovirus F protein, e.g., F proteins of the nucleopolyhedrovirus (NPV) genera, e.g., Spodoptera exigua MNPV (SeMNPV) F protein and Lymantria dispar MNPV (LdMNPV), and paramyxovirus F proteins.

In some embodiments, Class II viral membrane proteins include, but are not limited to, tick bone encephalitis E (TBEV E), Semliki Forest Virus E1/E2.

In some embodiments, Class III viral membrane fusion proteins include, but are not limited to, rhabdovirus G (e.g., fusogenic protein G of the Vesicular Stomatitis Virus (VSV-G)), herpesvirus glycoprotein B (e.g., Herpes Simplex virus 1 (HSV-1) gB)), Epstein Barr Virus glycoprotein B (EBV gB), thogotovirus G, baculovirus gp64 (e.g., Autographa California multiple NPV (AcMNPV) gp64), Baboon endogenous retrovirus envelope glycoprotein (BaEV), and Borna disease virus (BDV) glycoprotein (BDV G). In some embodiments, the fusogen is Baboon endogenous retrovirus envelope glycoprotein (BaEV) or a variant thereof.

Examples of other viral fusogens, e.g., membrane glycoproteins and viral fusion proteins, include, but are not limited to: viral syncytia proteins such as influenza hemagglutinin (HA) or mutants, or fusion proteins thereof; human immunodeficiency virus type 1 envelope protein (HIV-1 ENV), gp120 from HIV binding LFA-1 to form lymphocyte syncytium, HIV gp41, HIV gp160, or HIV Trans-Activator of Transcription (TAT); viral glycoprotein VSV-G, viral glycoprotein from vesicular stomatitis virus of the Rhabdoviridae family; glycoproteins gB and gH-gL of the varicella-zoster virus (VZV); murine leukemia virus (MLV)-10A1; Gibbon Ape Leukemia Virus glycoprotein (GaLV); type G glycoproteins in Rabies, Mokola, vesicular stomatitis virus and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of Measles virus; canine distemper virus, Newcastle disease virus, human parainfluenza virus 3, simian virus 41, Sendai virus and human respiratory syncytial virus; gH of human herpesvirus 1 and simian varicella virus, with the chaperone protein gL; human, bovine and cercopithicine herpesvirus gB; envelope glycoproteins of Friend murine leukemia virus and Mason Pfizer monkey virus; mumps virus hemagglutinin neuraminidase, and glycoproteins F1 and F2; membrane glycoproteins from Venezuelan equine encephalomyelitis; paramyxovirus F protein; SIV gp160 protein; Ebola virus G protein; or Sendai virus fusion protein, or a homologue thereof, a fragment thereof, a variant thereof, and a protein fusion comprising one or more proteins or fragments thereof.

Non-mammalian fusogens include viral fusogens, homologues thereof, fragments thereof, and fusion proteins comprising one or more proteins or fragments thereof. Viral fusogens include class I fusogens, class II fusogens, class III fusogens, and class IV fusogens. In embodiments, class I fusogens such as human immunodeficiency virus (HIV) gp41, have a characteristic post fusion conformation with a signature trimer of α-helical hairpins with a central coiled-coil structure. Class I viral fusion proteins include proteins having a central post fusion six-helix bundle. Class I viral fusion proteins include influenza HA, parainfluenza F, HIV Env, Ebola GP, hemagglutinins from orthomyxoviruses, F proteins from paramyxoviruses (e.g. Measles, (Katoh et al. BMC Biotechnology 2010, 10:37)), ENV proteins from retroviruses, and fusogens of filoviruses and coronaviruses. In embodiments, class II viral fusogens such as dengue E glycoprotein, have a structural signature of β-sheets forming an elongated ectodomain that refolds to result in a trimer of hairpins. In embodiments, the class II viral fusogen lacks the central coiled coil. Class II viral fusogen can be found in alphaviruses (e.g., E1 protein) and flaviviruses (e.g., E glycoproteins). Class II viral fusogens include fusogens from Semliki Forest virus, Sinbis, rubella virus, and dengue virus. In embodiments, class III viral fusogens such as the vesicular stomatitis virus G glycoprotein, combine structural signatures found in classes I and II. In embodiments, a class III viral fusogen comprises a helices (e.g., forming a six-helix bundle to fold back the protein as with class I viral fusogens), and β sheets with an amphiphilic fusion peptide at its end, reminiscent of class II viral fusogens. Class III viral fusogens can be found in rhabdoviruses and herpesviruses. In embodiments, class IV viral fusogens are fusion-associated small transmembrane (FAST) proteins (doi:10.1038/sj.emboj.7600767, Nesbitt, Rae L., “Targeted Intracellular Therapeutic Delivery Using Liposomes Formulated with Multifunctional FAST proteins” (2012). Electronic Thesis and Dissertation Repository. Paper 388), which are encoded by nonenveloped reoviruses. In embodiments, the class IV viral fusogens are sufficiently small that they do not form hairpins (doi: 10.1146/annurev-cellbio-101512-122422, doi:10.1016/j.devcel.2007.12.008).

Additional exemplary fusogens are disclosed in U.S. Pat. No. 9,695,446, US 2004/0028687, U.S. Pat. Nos. 6,416,997, 7,329,807, US 2017/0112773, US 2009/0202622, WO 2006/027202, and US 2004/0009604, the entire contents of all of which are hereby incorporated by reference.

In some embodiments, the fusogen is a poxviridae fusogen.

In some embodiments, the lipid particle comprises a fusogen that is a paramyxovirus envelope attachment proteins. In some embodiments, the lipid particle comprises a paramyxovirus envelope attachment protein exposed on the surface of the targeted lipid particle. In some embodiments, the lipid particle comprises a fusogen that is a retargeted attachment protein exposed on the surface of the targeted lipid particle.

In some embodiments, the paramyxovirus envelope attachment protein and/or retargeted attachment protein provided herein exhibit fusogenic activity to a target cell, such as to deliver an exogenous agent or nucleic acid exogenous agent to the target cell.

In some embodiments, the paramyxovirus attachment protein is or comprises a hemagglutinin-neuraminidase (HN) from a respiratory paramyxovirus. In some embodiments, the respiratory paramyxovirus is a Sendai virus. The HN glycoproteins of Sendai viruses function to attach to sialic acids via the HN protein, and to mediate cell fusion for entry to cells via the F (fusion) protein. In some embodiments, the paramyxovirus attachment protein is or comprises a HN protein from the murine parainfluenza virus type 1 (See e.g., U.S. patent Ser. No. 10/704,061).

In some embodiments, the paramyxovirus attachment protein is or comprises a Nipah virus protein G, a measles protein H, a tupaia paramyxovirus H protein, a paramyxovirus G protein, a paramyxovirus H protein, a paramyxovirus HN protein, a Morbillivirus H protein, a respirovirus HN protein, a Sendai HN protein, a rubulavirus HN protein, an avulavirus HN protein, or a derivative thereof. In some embodiments, the paramyxovirus attachment protein is or comprises a sequence chosen from Nipah virus G proteins, measles virus H proteins, tupaia paramyxovirus H proteins, paramyxovirus G proteins and H proteins and HN proteins, Hendra virus G proteins, Henipavirus G proteins, Morbillivirus H proteins, respirovirus HN protein, a Sendai virus HN protein, rubulavirus HN proteins, or avulavirus HN proteins, or a derivative thereof, or any combination thereof.

In some embodiments, the lipid particles, such as viral vectors or viral-like particles, contain one or more attachment proteins (e.g., fusogens). In some embodiments, the lipid particle, e.g. viral vector or viral-like particle, contains one or more exogenous or overexpressed attachment protein (e.g., fusogen). In some embodiments, the one or more fusogen is disposed in the lipid bilayer. In some embodiments, the one or more fusogen facilitates the fusion of the lipid particle to a membrane. In some embodiments, the lipid particle comprises one or more attachment proteins that are retargeted, such as an attachment protein that is linked to at least two targeting moieties, such as a first and second targeting moiety.

In some embodiments, the lipid particle comprises an attachment protein that is a first fusogen (e.g., paramyxovirus attachment protein) that is retargeted, such as a fusogen (e.g., paramyxovirus envelope attachment protein) that is linked to at least two targeting moieties, such as a first and second targeting moiety. In some embodiments, the lipid particle further comprises at least one fusion protein.

In some embodiments, the lipid particle comprises at least two fusogens (co-fusogens). In some embodiments, the lipid particle comprises at least two fusogens (e.g., paramyxovirus envelope attachment proteins as described below) exposed on the surface of the targeted lipid particle. In some embodiments, the lipid particle comprises at least two retargeted fusogens (e.g., attachment proteins) exposed on the surface of the targeted lipid particle. In some embodiments, the lipid particle further comprises at least one fusion protein.

In some embodiments, the fusogens (e.g., paramyxovirus envelope attachment proteins) and/or retargeted fusogens (e.g., attachment proteins provided herein) exhibit fusogenic activity to a target cell, such as to deliver an exogenous agent or nucleic acid exogenous agent to the target cell.

### A. Paramyxovirus Fusogen

In some embodiments, the lipid particle comprises a fusogen that is a paramyxovirus fusogen. In some embodiments, the lipid particle comprises a paramyxovirus fusogen exposed on the surface of the targeted lipid particle. In some embodiments, the lipid particle comprises a paramyxovirus fusogen that is a retargeted attachment protein exposed on the surface of the targeted lipid particle.

In some embodiments, the paramyxovirus fusogen and/or retargeted paramyxovirus fusogen provided herein exhibit fusogenic activity to a target cell, such as to deliver an exogenous agent or nucleic acid exogenous agent to the target cell.

In some embodiments, the paramyxovirus fusogen is or comprises a hemagglutinin-neuraminidase (HN) from a respiratory paramyxovirus. In some embodiments, the respiratory paramyxovirus is a Sendai virus. The HN glycoproteins of Sendai viruses function to attach to sialic acids via the HN protein, and to mediate cell fusion for entry to cells via the F (fusion) protein. In some embodiments, the paramyxovirus attachment protein is or comprises a HN protein from the murine parainfluenza virus type 1 (See e.g., U.S. patent Ser. No. 10/704,061).

In some embodiments, the paramyxovirus fusogen is or comprises a Nipah virus protein G, a measles protein H, a tupaia paramyxovirus H protein, a paramyxovirus G protein, a paramyxovirus H protein, a paramyxovirus HN protein, a Morbillivirus H protein, a respirovirus HN protein, a Sendai HN protein, a rubulavirus HN protein, an avulavirus HN protein, or a derivative thereof. In some embodiments, the paramyxovirus attachment protein is or comprises a sequence chosen from Nipah virus G proteins, measles virus H proteins, tupaia paramyxovirus H proteins, paramyxovirus G proteins and H proteins and HN proteins, Hendra virus G proteins, Henipavirus G proteins, Morbillivirus H proteins, respirovirus HN protein, a Sendai virus HN protein, rubulavirus HN proteins, or avulavirus HN proteins, or a derivative thereof, or any combination thereof.

In some embodiments, the fusogens may be derived from a paramyxovirus. In some embodiments, the fusogen includes a fusogen derived from a paramyxovirus envelope attachment protein, such as a G, H or HN protein, and at least one fusion (F) protein. In some embodiments, the attachment protein is a retargeted attachment protein that is fused to a targeting moiety directed against a target molecule on the surface of a cell. In some embodiments, an attachment protein can include a protein that is variant attachment protein containing one or more mutations (e.g. amino acid substitutions) to reduce or ablate the native tropism relative to the wild-type paramyxovirus envelope attachment protein not comprising the one or more mutations.

In other aspects, provided herein is a lipid particle in which a retargeted attachment protein includes a targeting moiety directed to a target molecule on the surface of the cells and the lipid particle, in some embodiments the attachment protein is a variant attachment protein that comprises one or more mutations to reduce native tropism relative to the wild-type paramyxovirus envelope attachment protein not comprising the one or more mutations. In some embodiments, the retargeted attachment protein comprises a paramyxovirus envelope attachment protein; and (ii) a targeting moiety directed to a target molecule on a T cell, such as CD4 or CD8.

In some embodiments, the targeting moiety, such as each of the first targeting moiety and the second targeting moiety, are independently selected from the group consisting of an antibody or antigen-binding fragment, an engineered binding domain, a nanobody, a DARPin, an Aptamer, an Affimer, an Affibody, a Knottin, an Avimer, a Monobody, an Anticalin, a Fynomer, and a targeting peptide.

In some embodiments, any of the provided lipid particles also contains at least one paramyxovirus fusion (F) protein or a biologically active portion thereof embedded in the lipid bilayer. In some embodiments, the F protein is from a Paramyxovirus, a Henipavirus (e.g., Hendra (HeV), Nipah (NiV) virus, Cedar henipavirus (CedV), Kumasi virus (KV), Möjiāng virus (MojV), or Langya virus), or is a biologically active portion thereof or is a variant or mutant thereof. In particular embodiments, the F protein is from a Nipah (NiV) virus.

In naturally occurring paramyxoviruses, the fusion (F) and attachment (G, H, or HN) glycoproteins mediate cellular entry of paramyxovirus, such as Nipah virus. In some embodiments, the combination of an F protein, such as a NiV-F protein, and variant NiV-G protein as provided herein is able to mediate cellular entry of a provided lipid particle (e.g. lentiviral vector).

The F protein, such as Nipah Virus F protein, also known as NiV-F, is a class I fusion protein that has structural and functional features in common with fusion proteins of many families (e.g., HIV-1 gp41 or influenza virus hemagglutinin [HA]), such as an ectodomain with a hydrophobic fusion peptide and two heptad repeat regions (White J M et al. 2008. Crit Rev Biochem Mol Biol 43:189-219). F proteins are synthesized as inactive precursors F₀ and are activated by proteolytic cleavage into the two disulfide-linked subunits F₁ and F₂ (Moll M. et al. 2004. J. Virol. 78(18): 9705-9712).

In some embodiments, the lipid particle comprises a paramyxovirus envelope attachment protein comprising a G protein. G proteins are attachment proteins of henipavirus (e.g. Nipah virus or Hendra virus) that are type II transmembrane glycoproteins containing an N-terminal cytoplasmic tail, a transmembrane domain, an extracellular stalk, and a globular head (Liu, Q. et al. 2015. Journal of Virology, 89(3):1838-1850). The Nipah virus attachment protein, NiV-G, recognizes the receptors EphrinB2 and EphrinB3. Binding of the receptor to NiV-G triggers a series of conformational changes that eventually lead to the triggering of NiV-F, which exposes the fusion peptide of NiV-F, allowing another series of conformational changes that lead to virus-cell membrane fusion (Stone J. A. et al. 2016. J Virol. 90(23): 10762-10773). EphrinB2 was previously identified as the primary NiV receptor (Negrete et al., 2005), as well as ephrinB3 as an alternate receptor (Negrete et al., 2006). In fact, wild-type NiV-G has a high affinity for ephrinB2 and B3, with affinity binding constants (Kd) in the picomolar range (Negrete et al., 2006) (Kd=0.06 nM and 0.58 nM for cell surface expressed ephrinB2 and B3, respectively). In some embodiments, the G protein is from a Paramyxovirus, a Henipavirus (e.g., Hendra (HeV), Nipah (NiV) virus, Cedar henipavirus (CedV), Kumasi virus (KV), Möjiāng virus (MojV), or Langya virus), or is a biologically active portion thereof or is a variant or mutant thereof. In particular embodiments, the G protein is from a Nipah (NiV) virus.

In some embodiments, the paramyxovirus envelope attachment protein comprising a G protein may be further linked to a targeting moiety as a retargeted attachment protein to facilitate specific targeting of the lipid particle to a target molecule for fusion with a desired target cell. Thus, the provided embodiments, the paramyxovirus envelope attachment protein comprising a G protein may be re-targeted as retargeted attachment proteins to any desired cell type for specific targeting of a lipid particle (e.g. lentiviral vector) and, in some cases, specific delivery to a target cell of a transgene or heterologous protein contained therein.

Thus, also provided herein are lipid particles containing a lipid bilayer enclosing a lumen or cavity and a paramyxovirus envelope attachment protein (e.g., G protein) containing an antigen binding domain or a biologically active portion thereof, such as a single domain antibody (sdAb) variable domain, in which the retargeted G glycoprotein is embedded in the lipid bilayer of the lipid particles. In particular embodiments, the binding domain is an antibody with the ability to bind, such as specifically bind, to a desired target molecule. Exemplary binding domains are described in Section III.A.1.B.

The efficiency of transduction of lipid particles can be improved by engineering mutations in one or both of NiV-F and NiV-G. Several such mutations have been previously described (see, e.g., Lee at al., 2011, Trends in Microbiology). This is useful, for example, for maintaining the specificity and picomolar affinity of NiV-G for ephrinB2 and/or B3. Additionally, mutations in NiV-G that completely abrogate ephrinB2 and B3 binding, but that do not impact the association of this NiV-G with NiV-F, have been identified (Aguilar, et al. J Biol Chem. 2009; 284(3):1628-1635.; Weise et al. J Virol. 2010; 84(15):7634-764; Negrete et al., J Virol. 2007; 81(19):10804-10814; Negrete et al. PLoS Pathog. 2006; Guillaume et al., J. Virol 2006, 80 (15) 7546-7554 In some cases, methods to improve targeting of lipid particles can be achieved by fusion of a binding molecule with a G protein (e.g. NiV-G, including a NiV-G with mutations to abrogate Ephrin B2 and Ephrin B3 binding). This does allow for altered G protein tropism allowing for targeting of other desired cell types that are not ephrinB2+ through the addition of the binding molecule directed against a different cell surface molecule. Thus, in provided aspects, a paramyxovirus envelope attachment protein is a variant NiV-G protein that may further contain a mutation to reduce or abrogate binding to Ephrin B2 and/B3. In some embodiments, the mutations can include one or more of mutations E501A, W504A, Q530A and E533A, with reference to numbering of wild-type NiV-G set forth in SEQ ID NO:1.

It has been reported that the henipavirus F proteins from various species exhibit compatibility with G proteins from other species to trigger fusion (Brandel-Tretheway et al. Journal of Virology. 2019. 93(13):e00577-19). In some aspects of the provided lipid particles (e.g. lentiviral vector), the F protein is heterologous to the G protein, i.e. the F and G protein or biologically active portions are from different henipavirus species. For example, the G protein is from Hendra virus and the F protein is a NiV-F as described. In other aspects, the F and/or G protein can be retargeted F and/or G protein containing regions of F and/or G proteins from different species of Henipavirus. In some embodiments, switching a region of amino acid residues of the F protein from one species of Henipavirus to another can result in fusion to the G protein of the species comprising the amino acid insertion. (Brandel-Tretheway et al. 2019). In some cases, the chimeric F and/or G protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus.

### 1. Paramyxovirus Attachment Proteins

In some embodiments, the lipid particles provided herein comprise a paramyxovirus envelope attachment protein. In some embodiments, the paramyxovirus envelope attachment protein may be derived from an envelope glycoprotein G, H and/or HN of the Paramyxoviridae family. In some embodiments, the paramyxovirus attachment protein may be a variant paramyxovirus attachment protein that contains one or more mutations to ablate natural tropism of the wildtype paramyxovirus attachment protein. In some embodiments, the paramyxovirus attachment protein, such as a variant paramyxovirus attachment protein, is retargeted.

In some embodiments, the lipid particles provided herein comprise more than one paramyxovirus envelope attachment protein, such as 1, 2 or 3 paramyxovirus envelope attachment protein. In some embodiments, the lipid particles provided herein comprise a first paramyxovirus envelope attachment protein, and a second paramyxovirus envelope attachment protein. In some embodiments, at least one of the paramyxovirus attachment protein is retargeted, such as by fusion with a targeting moiety. In some embodiments, such a retargeted paramyxovirus attachment protein also typically contains one or more mutations to ablate natural tropism of the wildtype paramyxovirus attachment protein. In some embodiments, at least one of the paramyxovirus attachment protein is modified not to exhibit target tropism, in which the protein contains one or more mutations to ablate natural tropism of the wildtype paramyxovirus attachment protein and in which such protein also is not fused with a targeting moiety. In some embodiments, the first and second paramyxovirus envelope attachment protein are displayed on the surface of the lipid particle, such as embedded in the lipid bilayer, as separate proteins. In some embodiments, the first and second paramyxovirus envelope attachment protein are a fusion protein in which the first and second paramyxovirus envelope attachment protein are linked in tandem, in some cases via a linker. In some embodiments, the paramyxovirus envelope attachment protein may be derived from an envelope glycoprotein G, H and/or HN of the Paramyxoviridae family.

In some embodiments, the lipid particles provided herein comprise a first paramyxovirus envelope attachment protein, a second paramyxovirus envelope attachment protein, and a third paramyxovirus envelope attachment protein. In some embodiments, at least two of the paramyxovirus attachment protein is retargeted, such as by fusion with a targeting moiety. In some embodiments, such a retargeted paramyxovirus attachment protein also typically contains one or more mutations to ablate natural tropism of the wildtype paramyxovirus attachment protein. In some embodiments, at least one of the paramyxovirus attachment protein is modified not to exhibit target tropism, in which the protein contains one or more mutations to ablate natural tropism of the wildtype paramyxovirus attachment protein and in which such protein also is not fused with a targeting moiety. In some embodiments, the first, second, and third paramyxovirus envelope attachment protein are each displayed on the surface of the lipid particle, such as embedded in the lipid bilayer, as separate proteins. In some embodiments, at least two of the paramyxovirus envelope attachment proteins are a fusion protein in which the two paramyxovirus envelope attachment protein are linked in tandem, in some cases via a linker. In some embodiments, each of the first, second, and third paramyxovirus envelope attachment protein may independently be derived from an envelope glycoprotein G, H and/or HN of the Paramyxoviridae family.

In some embodiments, the lipid particles provided herein comprise a first paramyxovirus envelope attachment protein, a second paramyxovirus envelope attachment protein, a third paramyxovirus envelope attachment protein, and one or more additional paramyxovirus envelope attachment proteins, such as a fourth paramyxovirus envelope attachment protein, or a fourth and fifth paramyxovirus envelope attachment protein, or a fourth, fifth, and sixth paramyxovirus envelope attachment protein, or beyond. In some embodiments, each of the paramyxovirus envelope attachment proteins may independently be an envelope glycoprotein G, H and/or HN of the Paramyxoviridae family.

### A) G Proteins

In some embodiments, the paramyxovirus envelope attachment protein is an attachment glycoprotein G (G protein) or biologically active portion thereof. In some embodiments, the paramyxovirus G protein may be a variant paramyxovirus G protein that contains one or more mutations to ablate natural tropism of the wildtype paramyxovirus G protein. In some embodiments, the paramyxovirus G protein is retargeted.

In some embodiments, the lipid particle comprises a retargeted attachment protein, exposed on the surface of the targeted lipid particle. In some embodiments, the retargeted attachment protein is or comprises a paramyxovirus attachment protein, wherein the paramyxovirus attachment protein is an attachment glycoprotein G (G protein) or biologically active portion thereof. In some embodiments, the retargeted attachment protein is or comprises a paramyxovirus attachment protein, wherein the paramyxovirus attachment protein is an attachment glycoprotein G (G protein) or biologically active portion thereof, and comprises a targeting moiety directed to a target molecule, e.g., a binding domain or a binding agent, expressed on the surface of a target cell.

In some embodiments, the paramyxovirus envelope attachment protein, first paramyxovirus envelope attachment protein, and/or second paramyxovirus envelope attachment protein and/or the third paramyxovirus envelope attachment protein and/or the fourth paramyxovirus envelope attachment protein and/or the fifth paramyxovirus envelope attachment protein and/or the sixth paramyxovirus envelope attachment protein, and/or any additional paramyxovirus envelope attachment protein is an attachment glycoprotein G (G protein) or biologically active portion thereof. In some embodiment, the retargeted attachment protein comprises a first paramyxovirus envelope attachment protein G.

In some embodiments, the lipid particle comprises a retargeted attachment protein, a first retargeted attachment protein, and/or second retargeted attachment protein exposed on the surface of the targeted lipid particle. In some embodiments, the lipid particle further comprises a third retargeted attachment protein exposed on the surface of the targeted lipid particle. In some embodiments, the lipid particle further comprises a third retargeted attachment protein and a fourth retargeted attachment protein exposed on the surface of the targeted lipid particle. In some embodiments, the lipid particle further comprises a third retargeted attachment protein, a fourth retargeted attachment protein, and a fifth retargeted attachment protein exposed on the surface of the targeted lipid particle. In some embodiments, the lipid particle further comprises a third retargeted attachment protein, a fourth retargeted attachment protein, a fifth retargeted attachment protein, and one or more additional retargeted attachment proteins, exposed on the surface of the targeted lipid particle. In some embodiments, the retargeted attachment protein is or comprises a paramyxovirus attachment protein, wherein the paramyxovirus attachment protein is an attachment glycoprotein G (G protein) or biologically active portion thereof. In some embodiments, the retargeted attachment protein is or comprises a paramyxovirus attachment protein, wherein the paramyxovirus attachment protein is an attachment glycoprotein G (G protein) or biologically active portion thereof, and comprises a targeting moiety directed to a target molecule, e.g., a binding domain or a binding agent, expressed on the surface of a target cell.

In some embodiments, the lipid particles provided herein comprise a first paramyxovirus envelope G protein, and a second paramyxovirus envelope G protein. In some embodiments, the paramyxovirus attachment protein is retargeted, such as by fusion with a targeting moiety. In some embodiments, such a retargeted paramyxovirus G protein also typically contains one or more mutations to ablate natural tropism of the wildtype paramyxovirus G protein. In some embodiments, at least one of the paramyxovirus G protein is modified not to exhibit target tropism, in which the protein contains one or more mutations to ablate natural tropism of the wildtype paramyxovirus G protein and in which such protein also is not fused with a targeting moiety. In some embodiments, the first and second paramyxovirus envelope G protein are displayed on the surface of the lipid particle, such as embedded in the lipid bilayer, as separate proteins. In some embodiments, the first and second paramyxovirus envelope G protein are a fusion protein in which the first and second paramyxovirus envelope G protein are linked in tandem, in some cases via a linker.

In some embodiments, the lipid particles provided herein comprise a first paramyxovirus envelope G protein, a second paramyxovirus envelope G protein, and a third paramyxovirus envelope G protein. In some embodiments, at least two of the paramyxovirus G protein is retargeted, such as by fusion with a targeting moiety. In some embodiments, such a retargeted paramyxovirus G protein also typically contains one or more mutations to ablate natural tropism of the wildtype paramyxovirus G protein. In some embodiments, at least one of the paramyxovirus G protein is modified not to exhibit target tropism, in which the protein contains one or more mutations to ablate natural tropism of the wildtype paramyxovirus G protein and in which such protein also is not fused with a targeting moiety. In some embodiments, the first, second, and third paramyxovirus envelope G protein are each displayed on the surface of the lipid particle, such as embedded in the lipid bilayer, as separate proteins. In some embodiments, at least two of the paramyxovirus envelope G proteins are a fusion protein in which the two paramyxovirus envelope G protein are linked in tandem, in some cases via a linker.

The envelope attachment G proteins are type II transmembrane glycoproteins containing an N-terminal cytoplasmic tail (e.g. corresponding to amino acids 1-49 of SEQ ID NO:1), a transmembrane domain (e.g. corresponding to amino acids 50-70 of SEQ ID NO:1), and an extracellular domain containing an extracellular stalk (e.g. corresponding to amino acids 71-187 of SEQ ID NO:1), and a globular head (corresponding to amino acids 188-602 of SEQ ID NO:1). The N-terminal cytoplasmic domain is within the inner lumen of the lipid bilayer and the C-terminal portion is the extracellular domain that is exposed on the outside of the lipid bilayer. Regions of the stalk in the C-terminal region (e.g. corresponding to amino acids 71-187 of SEQ ID NO: 1) have been shown to be involved in interactions with F protein and triggering of F protein fusion (Liu et al. 2015 J of Virology 89:1838). In wild-type G protein, the globular head mediates receptor binding to henipavirus entry receptors Ephrin B2 and Ephrin B3, but is dispensable for membrane fusion (Brandel-Tretheway et al. Journal of Virology. 2019. 93(13)e00577-19).

In some embodiments herein, tropism of the G protein is altered by linkage of the G protein or biologically active fragment thereof (e.g. cytoplasmic truncation) to a sdAb variable domain. Binding of the G protein to a binding partner can trigger fusion mediated by a compatible paramyxovirus fusion protein (e.g., F protein) or biologically active portion thereof (such as any of the F proteins described in II.B below). G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved between henipavirus species. For example, the G protein of NiV and HeV viruses share 79% amino acid identity. Studies have shown a high degree of compatibility among G proteins with F proteins of different species as demonstrated by heterotypic fusion activation (Brandel-Tretheway et al. Journal of Virology. 2019). As described, a lipid particle can contain at least two envelope attachment proteins (e.g., co-fusogens). In particular embodiments, the F protein or the functionally active variant or biologically active portion thereof retains fusogenic activity in conjunction with the at least two envelope attachment proteins (e.g., co-fusogens that are paramyxovirus attachment protein Gs) as provided, such as any set forth below. Fusogenic activity includes the activity of the paramyxovirus fusion protein (e.g., F protein) in conjunction with a G protein to promote or facilitate fusion of two membrane lumens, such as the lumen of the lipid particle provided herein (e.g. having embedded in its lipid bilayer, such as exposed on its surface, at least two G proteins and a F protein), and a cytoplasm of a target cell, e.g. a cell that contains a surface receptor or molecule that is recognized or bound by the G protein.

### Exemplary Henipavirus Protein G Sequences are Provided in Table 2

Table 2. Henipavirus protein G sequence clusters. Column 1, Genbank ID includes the Genbank ID of the whole genome sequence of the virus that is the centroid sequence of the cluster. Column 2, nucleotides of CDS provides the nucleotides corresponding to the CDS of the gene in the whole genome. Column 3, Full Gene Name, provides the full name of the gene including Genbank ID, virus species, strain, and protein name. Column 4, Sequence, provides the amino acid sequence of the gene. Column 5, #Sequences/Cluster, provides the number of sequences that cluster with this centroid sequence. Column 6 provides the SEQ ID numbers for the described sequences.

TABLE 2
Exemplary Henipavirus protein G sequences

| Gen | Nucleotides | | | SEQ |
|---|---|---|---|---|
| bank | of | Full sequence | | ID |
| ID | CDS | ID | Sequence | NO |
| AF017149 | 8913-10727 | gb: AF017149\| | MMADSKLVSLNNNLSGKIKDQGKVIKNYYGT | 561 |
| | | Organism: Hendra | MDIKKINDGLLDSKILGAFNTVIALLGSIIIIVM | |
| | | virus\|Strain | NIMIIQNYTRTTDNQALIKESLQSVQQQIKALTD | |
| | | Name: | KIGTEIGPKVSLIDTSSTITIPANIGLLGSKISQS | |
| | | UNKNOWN- | TSSINENVNDKCKFTLPPLKIHECNISCPNPLPF | |
| | | AF017149\| | REYRPISQGVSDLVGLPNQICLQKTTSTILKPR | |
| | | Protein | LISYTLPINTREGVCITDPLLAVDNGFFAYSHL | |
| | | Name: | EKIGSCTRGIAKQRIIGVGEVLDRGDKVPSMF | |
| | | glycoprotein\| | MTNVWTPPNPSTIHHCSSTYHEDFYYTLCAVS | |
| | | Gene Symbol: G | HVGDPILNSTSWTESLSLIRLAVRPKSDSGDYN | |
| | | | QKYIAITKVERGKYDKVMPYGPSGIKQGDTLY | |
| | | | FPAVGFLPRTEFQYNDSNCPIIHCKYSKAENCR | |
| | | | LSMGVNSKSHYILRSGLLKYNLSLGGDIILQFI | |
| | | | EIADNRLTIGSPSKIYNSLGQPVFYQASYSWDT | |
| | | | MIKLGDVDTVDPLRVQWRNNSVISRPGQSQC | |
| | | | PRFNVCPEVCWEGTYNDAFLIDRLNWVSAGV | |
| | | | YLNSNQTAENPVFAVFKDNEILYQVPLAEDDT | |
| | | | NAQKTITDCFLLENVIWCISLVEIYDTGDSVIRP | |
| | | | KLFAVKIPAQCSES | |
| AF212302 | 8943-10751 | gb: AF212302\| | MPAENKKVRFENTTSDKGKIPSKVIKSYYGTMD | 1 |
| | | Organism: Nipah | IKKINEGLLDSKILSAFNTVIALLGSIVIIVMNI | |
| | | virus\|Strain | MIIQNYTRSTDNQAVIKDALQGIQQQIKGLADKI | |
| | | Name: | GTEIGPKVSLIDTSSTITIPANIGLLGSKISQST | |
| | | UNKNOWN- | ASINENVNEKCKFTLPPLKIHECNISCPNPLPFR | |
| | | AF212302\| | EYRPQTEGVSNLVGLPNNICLQKTSNQILKPKL | |
| | | Protein | ISYTLPVVGQSGTCITDPLLAMDEGYFAYSHL | |
| | | Name: | ERIGSCSRGVSKQRIIGVGEVLDRGDEVPSLFM | |
| | | attachment | TNVWTPPNPNTVYHCSAVYNNEFYYVLCAVS | |
| | | glycoprotein\| | TVGDPILNSTYWSGSLMMTRLAVKPKSNGGG | |
| | | Gene Symbol: G | YNQHQLALRSIEKGRYDKVMPYGPSGIKQGD | |
| | | | TLYFPAVGFLVRTEFKYNDSNCPITKCQYSKP | |
| | | | ENCRLSMGIRPNSHYILRSGLLKYNLSDGENP | |
| | | | KVVFIEISDQRLSIGSPSKIYDSLGQPVFYQASF | |
| | | | SWDTMIKFGDVLTVNPLVVNWRNNTVISRPG | |
| | | | QSQCPRFNTCPEICWEGVYNDAFLIDRINWISA | |
| | | | GVFLDSNQTAENPVFTVFKDNEILYRAQLASE | |
| | | | DTNAQKTITNCFLLKNKIWCISLVEIYDTGDN | |
| | | | VIRPKLFAVKIPEQCT | |
| JQ001776 | 8170-10275 | gb: JQ001776: | MLSQLQKNYLDNSNQQGDKMNNPDKKLSVN | 562 |
| | | 8170-10275\| | FNPLELDKGQKDLNKSYYVKNKNYNVSNLLNES | |
| | | Organism: Cedar | LHDIKFCIYCIFSLLIIITIINIITISIVITRLKV | |
| | | virus\|Strain | HEENNGMESPNLQSIQDSLSSLTNMINTEITPRI | |
| | | Name: CG1a\| | GILVTATSVTLSSSINYVGTKTNQLVNELKDYIT | |
| | | Protein | KSCGFKVPELKLHECNISCADPKISKSAMYST | |
| | | Name: | NAYAELAGPPKIFCKSVSKDPDFRLKQIDYVIP | |
| | | attachment | VQQDRSICMNNPLLDISDGFFTYIHYEGINSCK | |
| | | glycoprotein\| | KSDSFKVLLSHGEIVDRGDYRPSLYLLSSHYH | |
| | | Gene Symbol: G | PYSMQVINCVPVTCNQSSFVFCHISNNTKTLD | |
| | | | NSDYSSDEYYITYFNGIDRPKTKKIPINNMTAD | |
| | | | NRYIHFTFSGGGGVCLGEEFIIPVTTVINTDVFT | |
| | | | HDYCESFNCSVQTGKSLKEICSESLRSPTNSSR | |
| | | | YNLNGIMIISQNNMTDFKIQLNGITYNKLSFGS | |
| | | | PGRLSKTLGQVLYYQSSMSWDTYLKAGFVEK | |
| | | | WKPFTPNWMNNTVISRPNQGNCPRYHKCPEI | |
| | | | CYGGTYNDIAPLDLGKDMYVSVILDSDQLAE | |
| | | | NPEITVFNSTTILYKERVSKDELNTRSTTTSCFL | |
| | | | FLDEPWCISVLETNRFNGKSIRPEIYSYKIPKYC | |
| NC_025256 | 9117-11015 | gb: NC_025256: | MPQKTVEFINMNSPLERGVSTLSDKKTLNQSK | 563 |
| | | 9117-11015\| | ITKQGYFGLGSHSERNWKKQKNQNDHYMTV | |
| | | Organism: Bat | STMILEILVVLGIMFNLIVLTMVYYQNDNINQR | |
| | | Paramyxovirus | MAELTSNITVLNLNLNQLTNKIQREIIPRITLIDT | |
| | | Eid_hel/GH- | ATTITIPSAITYILATLTTRISELLPSINQKCEFK | |
| | | M74a/GHA/20 | TPTLVLNDCRINCTPPLNPSDGVKMSSLATNL | |
| | | 09\|Strain | VAHGPSPCRNFSSVPTIYYYRIPGLYNRTALDE | |
| | | Name: BatPV/ | RCILNPRLTISSTKFAYVHSEYDKNCTRGFKYY | |
| | | Eid_hel/GH- | ELMTFGEILEGPEKEPRMFSRSFYSPTNAVNY | |
| | | M74a/GHA/20 | HSCTPIVTVNEGYFLCLECTSSDPLYKANLSNS | |
| | | 09\|Protein | TFHLVILRHNKDEKIVSMPSFNLSTDQEYVQII | |
| | | Name: | PAEGGGTAESGNLYFPCIGRLLHKRVTHPLCK | |
| | | glycoprotein\| | KSNCSRTDDESCLKSYYNQGSPQHQVVNCLIR | |
| | | Gene | IRNAQRDNPTWDVITVDLTNTYPGSRSRIFGSF | |
| | | Symbol: G | SKPMLYQSSVSWHTLLQVAEITDLDKYQLDW | |
| | | | LDTPYISRPGGSECPFGNYCPTVCWEGTYNDV | |
| | | | YSLTPNNDLFVTVYLKSEQVAENPYFAIFSRD | |
| | | | QILKEFPLDAWISSARTTTISCFMFNNEIWCIAA | |
| | | | LEITRLNDDIIRPIYYSFWLPTDCRTPYPHTGK | |
| | | | MTRVPLRSTYNY | |
| NC_025352 | 8716-11257 | gb: NC_025352: | MATNRDNTITSAEVSQEDKVKKYYGVETAEK | 564 |
| | | 8716-11257\| | VADSISGNKVFILMNTLLILTGAIITITLNITNLT | |
| | | Organism: | AAKSQQNMLKIIQDDVNAKLEMFVNLDQLVK | |
| | | Mojiang | GEIKPKVSLINTAVSVSIPGQISNLQTKFLQKY | |
| | | virus\|Strain | VYLEESITKQCTCNPLSGIFPTSGPTYPPTDKPD | |
| | | Name: Tongguan1 | DDTTDDDKVDTTIKPIEYPKPDGCNRTGDHFT | |
| | | \|Protein | MEPGANFYTVPNLGPASSNSDECYTNPSFSIGS | |
| | | Name: | SIYMFSQEIRKTDCTAGEILSIQIVLGRIVDKGQ | |
| | | attachment | QGPQASPLLVWAVPNPKIINSCAVAAGDEMG | |
| | | glycoprotein\| | WVLCSVTLTAASGEPIPHMFDGFWLYKLEPDT | |
| | | Gene Symbol: G | EVVSYRITGYAYLLDKQYDSVFIGKGGGIQKG | |
| | | | NDLYFQMYGLSRNRQSFKALCEHGSCLGTGG | |
| | | | GGYQVLCDRAVMSFGSEESLITNAYLKVNDL | |
| | | | ASGKPVIIGQTFPPSDSYKGSNGRMYTIGDKY | |
| | | | GLYLAPSSWNRYLRFGITPDISVRSTTWLKSQ | |
| | | | DPIMKILSTCTNTDRDMCPEICNTRGYQDIFPL | |
| | | | SEDSEYYTYIGITPNNGGTKNFVAVRDSDGHI | |
| | | | ASIDILQNYYSITSATISCFMYKDEIWCIAITEG | |
| | | | KKQKDNPQRIYAHSYKIRQMCYNMKSATVTV | |
| | | | GNAKNITIRRY | |

In some embodiments, at least one G protein has a sequence set forth in any of SEQ ID NOS: 1, 561, 562, 563, or 564 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOS: 1, 561, 562, 563, or 564.

In particular embodiments, the paramyxovirus envelope attachment protein (e.g., G protein) or functionally active variant or biologically active portion is a protein that retains fusogenic activity in conjunction with a paramyxovirus fusion protein (e.g., F protein), such as a NiV-F protein described herein. Fusogenic activity includes the activity of the paramyxovirus envelope attachment protein (e.g., G protein) in conjunction with a paramyxovirus fusion protein (e.g., F protein) to promote or facilitate fusion of two membrane lumens, such as the lumen of the targeted lipid particle having embedded in its lipid bilayer a paramyxovirus fusion protein (e.g., F protein) and paramyxovirus envelope attachment protein (e.g., G protein), and a cytoplasm of a target cell, e.g. a cell that contains a surface receptor or molecule that is recognized or bound by the targeted envelope protein. In some embodiments, the paramyxovirus fusion protein (e.g., F protein) and the paramyxovirus envelope attachment protein (e.g., G protein) are from the same paramyxovirus species (e.g. the same Henipavirus species such as NiV-G and NiV-F).

In some embodiments, the NiV-G is a variant NiV-G proteins that contain an altered cytoplasmic tail compared to native NiV-G (e.g. SEQ ID NO:5) that are or can be incorporated into a lipid particle, such as a viral particle, including a lentiviral particle or lentiviral-like particle. The cytoplasmic tail of NiV-G corresponds to amino acids 1-45 of SEQ ID NO:5. In some cases, it is understood that the N-terminal methionine of NiV-G, or a variant NiV-G, as described herein can be cleaved and the cytoplasmic tail lacks an initial N-terminal methionine. For instance, in some embodiments, the cytoplasmic tail of wild-type NiV-G may correspond to amino acids 2-45 of SEQ ID NO:5, and the variant NiV-G protein contains a cytoplasmic tail that is altered compared to amino acids 2-45 of SEQ ID NO:5. In some embodiments, the variant NiV-G contains a modified cytoplasmic tail in which the native cytoplasmic tail is truncated or is replaced by a heterologous cytoplasmic tail.

Non-limiting examples of variant NiV-G proteins, including truncated NiV-G or NiV-G with a altered or modified cytoplasmic tail, are described in WO2013148327, WO2017182585, or PCT/US2022/081872. Further exemplary variant NiV-G proteins are described in Bender et al. 2016 PLoS Pathol 12(6):e1005641.

In some embodiments, at least one G protein is a variant G protein that is a functionally active variant or biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference G protein sequence. In some embodiments, the reference G protein sequence is the wild-type sequence of a G protein or a biologically active portion thereof. In some embodiments, at least one functionally active variant or the biologically active portion thereof is a variant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein or biologically active portion thereof. In some embodiments, the wild-type G protein has the sequence set forth in any one of SEQ ID NOS: 1, 561, 562, 563, or 564.

In some embodiments, at least one G protein is a variant G protein that is a biologically active portion that is an N-terminally and/or C-terminally truncated fragment of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein. In particular embodiments, the truncation is an N-terminal truncation of all or a portion of the cytoplasmic domain. In some embodiments, at least one variant G protein is a biologically active portion that is truncated and lacks up to 49 contiguous amino acid residues at or near the N-terminus of the wild-type G protein, such as a wild-type G protein set forth in any one of SEQ ID NOS: 1, 561, 562, 563, or 564. In some embodiments, at least one variant G protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the N-terminus of the wild-type G protein.

In some embodiments, at least one G protein is a wild-type Nipah virus G (NiV-G) protein or a Hendra virus G protein, or is a functionally active variant or biologically active portion thereof. In some embodiments, at least one G protein is a NiV-G protein that has the sequence set forth in SEQ ID NO:1, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:1.

In some embodiments, at least one G protein is a variant NiV-G that comprises a modified cytoplasmic tail which comprises a truncated cytoplasmic tail from a glycoprotein from the same Nipah virus. In some embodiments, the variant NiV-G contains a modified cytoplasmic tail in which at least a part of the native cytoplasmic tail (e.g. corresponding to amino acids 1-45 of SEQ ID NO:5) is a truncated portion thereof from a glycoprotein from Nipah Virus. In some embodiments, the cytoplasmic tail is a truncated portion thereof that is at least 5 amino acids in length. from or from about 5-44, from or from about 5-40, from or from about 5-30, from or from about 5-20, from or from about 5-10, from or from about 10-44, from or from about 10-40, from or from about 10-30, from or from about 10-20, from or from about 20-44, from or from about 20-40, from or from about 20-30, from or from about 30-44, from or from about 30-40, from or from about 40-44 amino acids in length. In some embodiments, the truncated portion thereof is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 or 44 amino acids in length. In some embodiments, the variant NiV-G has a cytoplasmic tail that is a truncated NiV-G cytoplasmic tail. In some embodiments, the truncated NiV-G cytoplasmic tail has a deletion of up to 40, up to 35, up to 30, up to 29, up to 28, up to 27, up to 26, up to 25, up to 24, up to 23, up to 22, up to 21, up to 20, up to 19, up to 18, up to 17, up to 16, up to 15, or up to 14 contiguous amino acid residues at or near the N-terminus of the wild-type NiV-G cytoplasmic tail set forth in SEQ ID NO: 28. In some embodiments, the variant NiV-G has a deletion of between 5 and 41 contiguous amino acid residues at or near the N-terminus of the wild-type NiV-G protein cytoplasmic tail set forth in SEQ ID NO: 4. In some embodiments, the variant NiV-G has a deletion of between 26 and 40 contiguous amino acid residues at or near the N-terminus of the wild-type NiV-G protein cytoplasmic tail set forth in SEQ ID NO: 4.

In some embodiments, at least one G protein is a variant NiV-G protein that is a biologically active portion of a wild-type NiV-G. In some embodiments, the biologically active portion is an N-terminally truncated fragment. In some embodiments, the variant NiV-G protein is truncated and lacks up to 5 contiguous amino acid residues at or near the N-terminus of the wild-type NiV-G protein, such as compared to wild-type NiV-G set forth in SEQ ID NO: 1. In some embodiments, the variant NiV-G protein is truncated and lacks up to 10 contiguous amino acid residues at or near the N-terminus of the wild-type NiV-G protein, such as compared to wild-type NiV-G set forth in SEQ ID NO: 1. In some embodiments, the variant NiV-G protein is truncated and lacks up to 15 contiguous amino acid residues at or near the N-terminus of the wild-type NiV-G protein, such as compared to wild-type NiV-G set forth in SEQ ID NO: 1. n some embodiments, the variant NiV-G protein is truncated and lacks up to 20 contiguous amino acid residues at or near the N-terminus of the wild-type NiV-G protein, such as compared to wild-type NiV-G set forth in SEQ ID NO: 1. In some embodiments, the variant NiV-G protein is truncated and lacks up to 25 contiguous amino acid residues at or near the N-terminus of the wild-type NiV-G protein, such as compared to wild-type NiV-G set forth in SEQ ID NO: 1. In some embodiments, the variant NiV-G protein is truncated and lacks up to 30 contiguous amino acid residues at or near the N-terminus of the wild-type NiV-G protein, such as compared to wild-type NiV-G set forth in SEQ ID NO: 1. In some embodiments, the variant NiV-G protein is truncated and lacks up to 35 contiguous amino acid residues at or near the N-terminus of the wild-type NiV-G protein, such as compared to wild-type NiV-G set forth in SEQ ID NO: 1. In some embodiments, the variant NiV-G protein (also called variant NiV-G) contains an N-terminal methionine.

In some embodiments, the variant NiV-G has a cytoplasmic tail deletion of amino acid residues 2-41, 2-40, 2-39, 2-38, 2-37, 2-36, 2-34, 2-35, 2-33, 2-32, 2-31, 2-30, 2-29, 2-28, 2-27, 2-26, 2-25, 2-22, 2-21, 2-16, 2-11, or 2-5 of SEQ ID NO:4. In some embodiments, the cytoplasmic tail is a truncated portion of the Nipah virus cytoplasmic tail set forth in any one of SEQ ID NOS: 6-28. In some embodiments, the cytoplasmic tail is a truncated portion of the Nipah virus cytoplasmic tail set forth in any one of SEQ ID NOS: 6-28 that lacks the N-terminal methionine. In some embodiments, the variant NiV-G has a sequence in which the cytoplasmic tail, such as set forth in any one of SEQ ID NOS: 6-28, is directly linked to the N-terminus of the sequence set forth in SEQ ID NO: 2. In some embodiments, the variant NiV-G has a sequence in which the cytoplasmic tail set forth in any one of SEQ ID NOS: 6-28 is directly linked to the N-terminus of the sequence set forth in SEQ ID NO: 3. In some of any such embodiments, the cytoplasmic tail is set forth in SEQ ID NO:7, 13, or 19.

In some embodiments, the truncated NiV-G comprises the sequence of amino acids set forth in SEQ ID NO: 211, 220 or 221, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOs: 211, 220 or 221. In some embodiments, the variant NiV-G is the sequence of amino acids set forth in SEQ ID NO: 211, 220 or 221.

In some embodiments, the variant NiV-G comprises a modified cytoplasmic tail which comprises a heterologous cytoplasmic tail or a truncated portion thereof from a glycoprotein from another virus. In some embodiments, the other virus is a member of the Kingdom Orthornavirae. In some embodiments, the other virus is a member of the family Paramyxoviridae, Rhabdoviridae, Arenaviridae, or Retroviridae. In some embodiments, the other virus is a member of the family Paramyxoviridae.

In some embodiments, the variant NiV-G contains a modified cytoplasmic tail in which at least a part of the native cytoplasmic tail (e.g. corresponding to amino acids 1-45 of SEQ ID NO:5) is replaced by a heterologous cytoplasmic tail or a truncated portion thereof from a glycoprotein from another virus from another virus or viral-associated protein. In some embodiments, the replaced cytoplasmic tail is a heterologous cytoplasmic tail or a truncated portion thereof that is at least 5 amino acids in length. In some embodiments, the replaced heterologous cytoplasmic tail or a truncated portion thereof is from or from about 5-180 amino acids in length, such as from or from about 5-150, from or from about 5-100, from or from about 5-75, from or from about 5-50, from or from about 5-40, from or from about 5-30, from or from about 5-20, from or from about 5-10, from or from about 10-150, from or from about 10-100, from or from about 10-75, from or from about 10-50, from or from about 10-40, from or from about 10-30, from or from about 10-20, from or from about 20-150, from or from about 20-100, from or from about 20-75, from or from about 20-50, from or from about 20-40, from or from about 20-30, from or from about 30-150, from or from about 30-100, from or from about 30-75, from or from about 30-50, from or from about 30-40, from or from about 40-150, from or from about 40-100, from or from about 40-75, from or from about 40-50, from or from about 50-150, from or from about 50-100, from or from about 50-75, from or from about 75-150, from or from about 75-100 or from or from about 100-150 amino acids in length. In some embodiments, the replaced heterologous cytoplasmic tail or a truncated portion thereof is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids in length. In some embodiments, the heterologous cytoplasmic tail or the truncated portion thereof is directly linked to the N-terminus of the sequence set forth in SEQ ID NO: 2. In some embodiments, the heterologous cytoplasmic tail or the truncated portion thereof is directly linked to the N-terminus of the sequence set forth in SEQ ID NO: 3.

In some embodiments, the heterologous cytoplasmic tail is a cytoplasmic tail or a truncated portion thereof from a glycoprotein from another virus, such as a paramyxovirus, a retrovirus, a filovirus, a rhabdovirus or an arenavirus. In some embodiments, the virus is a paramyxovirus other than a Nipah virus. For instance, the virus is a measles virus, Bat paramyxovirus, Cedar Virus, Canine Distemper Virus, Sendai virus, Hendra virus, Human Parainfluenza virus, or Newcastle Disease virus. In some embodiments, the replaced heterologous cytoplasmic tail is the native cytoplasmic tail or a truncated portion of the native cytoplasmic tail of another virus, such as a truncated portion of the cytoplasmic tail set forth in any one of SEQ ID NOS: 40-166. In some embodiments, the variant NiV-G has a sequence in which the heterologous cytoplasmic tail or the truncated portion thereof set forth in any one of SEQ ID NOS: 40-166 is directly linked to the N-terminus of the sequence set forth in SEQ ID NO: 2. In some embodiments, the variant NiV-G contains mutations in the extracellular domain that reduce or abrogate binding to an Ephrin B2 or B3 corresponding to one or more of E501A, W504A, Q530A and E533A, with numbering of residues as set forth SEQ ID NO:1. In some embodiments, the variant NiV-G has a sequence in which the heterologous cytoplasmic tail or the truncated portion thereof set forth in any one of SEQ ID NOS: 40-166 is directly linked to the N-terminus of the sequence set forth in SEQ ID NO: 3. In some embodiments, it is understood that the heterologous cytoplasmic tail or the truncated portion thereof may include any sequence set forth in any one of SEQ ID NOS: 40-166 that lacks the N-terminal methionine.

In some embodiments, the virus is a retrovirus. For instance, the virus may be a baboon endogenous virus (BaEV), Gibbon Ape Leukemia virus (GaLV), murine leukemia virus, or human immunodeficiency virus 1 (HIV-1). In some embodiments, the replaced heterologous cytoplasmic tail is the native cytoplasmic tail or a truncated portion of the native cytoplasmic tail of another virus, such as set forth in any one of SEQ ID NOS: 167-168, 174-177, 179-182, or 185-199. In some embodiments, the variant NiV-G has a sequence in which the heterologous cytoplasmic tail or the truncated portion thereof set forth in any one of SEQ ID NOS: 167-168, 174-177, 179-182, or 185-199 is directly linked to the N-terminus of the sequence set forth in SEQ ID NO: 2. In some embodiments, the variant NiV-G contains mutations in the extracellular domain that reduce or abrogate binding to an Ephrin B2 or B3 corresponding to one or more of E501A, W504A, Q530A and E533A, with numbering of residues as set forth SEQ ID NO:1. In some embodiments, the variant NiV-G has a sequence in which the heterologous cytoplasmic tail or the truncated portion thereof set forth in any one of SEQ ID NOS: 167-168, 174-177, 179-182, or 185-199 is directly linked to the N-terminus of the sequence set forth in SEQ ID NO: 3. In some embodiments, it is understood that the heterologous cytoplasmic tail or the truncated portion thereof may include any sequence set forth in any one of SEQ ID NOS: 167-168, 174-177, 179-182, or 185-199 that lacks the N-terminal methionine.

In some embodiments, the virus is a filovirus. For instance, the virus may be an Ebola virus (EboV). In some embodiments, the replaced heterologous cytoplasmic tail is the native cytoplasmic tail or a truncated portion of the native cytoplasmic tail of another virus, such as set forth in any one of SEQ ID NOS: 172 or 173. In some embodiments, the variant NiV-G has a sequence in which the heterologous cytoplasmic tail or the truncated portion thereof set forth in any one of SEQ ID NOS: 172 or 173 is directly linked to the N-terminus of the sequence set forth in SEQ ID NO: 2. In some embodiments, the variant NiV-G contains mutations in the extracellular domain that reduce or abrogate binding to an Ephrin B2 or B3 corresponding to one or more of E501A, W504A, Q530A and E533A, with numbering of residues as set forth SEQ ID NO:1. In some embodiments, the variant NiV-G has a sequence in which the heterologous cytoplasmic tail or the truncated portion thereof set forth in any one of SEQ ID NOS: 172 or 173 is directly linked to the N-terminus of the sequence set forth in SEQ ID NO: 3. In some embodiments, it is understood that the heterologous cytoplasmic tail or the truncated portion thereof may include any sequence set forth in any one of SEQ ID NOS: 172 or 173 that lacks the N-terminal methionine.

In some embodiments, the virus is a rhabdovirus. For instance, the virus may be Cocal vesiculovirus (Cocal) or vesicular stomatitis virus (VSV). In some embodiments, the replaced heterologous cytoplasmic tail is the native cytoplasmic tail or a truncated portion of the native cytoplasmic tail of another virus, such as set forth in any one of SEQ ID NOS: 170, 171, 183, or 184. In some embodiments, the variant NiV-G has a cytoplasmic tail in which the heterologous cytoplasmic tail or the truncated portion thereof set forth in any one of SEQ ID NOS: 70, 171, 183, or 184 is directly linked to the N-terminus of the sequence set forth in SEQ ID NO: 2. In some embodiments, the variant NiV-G contains mutations in the extracellular domain that reduce or abrogate binding to an Ephrin B2 or B3 corresponding to one or more of E501A, W504A, Q530A and E533A, with numbering of residues as set forth SEQ ID NO:1. In some embodiments, variant NiV-G has a sequence in which the heterologous cytoplasmic tail or the truncated portion thereof set forth in any one of SEQ ID NOS: 70, 171, 183, or 184 is directly linked to the N-terminus of the sequence set forth in SEQ ID NO: 3. In some embodiments, it is understood that the heterologous cytoplasmic tail or the truncated portion thereof may include any sequence set forth in any one of SEQ ID NOS: 70, 171, 183 or 184 that lacks the N-terminal methionine.

In some embodiments, the virus is an arenavirus. For instance, the virus may be Lymphocytic choriomeningitis virus (LCMV). In some embodiments, the replaced heterologous cytoplasmic tail is the native cytoplasmic tail or a truncated portion of the native cytoplasmic tail of another virus, such as set forth in SEQ ID NOS: 178. In some embodiments, the variant NiV-G has a sequence in which the heterologous cytoplasmic tail or the truncated portion thereof set forth in SEQ ID NOS: 178 is directly linked to the N-terminus of the sequence set forth in SEQ ID NO: 2. In some embodiments, the variant NiV-G contains mutations in the extracellular domain that reduce or abrogate binding to an Ephrin B2 or B3 corresponding to one or more of E501A, W504A, Q530A and E533A, with numbering of residues as set forth SEQ ID NO:1. In some embodiments, the variant NiV-G has a sequence in which the heterologous cytoplasmic tail or the truncated portion thereof set forth in SEQ ID NOS: 178 is directly linked to the N-terminus of the sequence set forth in SEQ ID NO: 3. In some embodiments, it is understood that the heterologous cytoplasmic tail or the truncated portion thereof may include any sequence set forth in any one of SEQ ID NOS: 178 that lacks the N-terminal methionine.

In some embodiments, at least one variant NiV-G protein is truncated and lacks up to amino acid 34 at or near the N-terminus of the wild-type NiV-G protein, such as compared to wild-type NiV-G set forth in SEQ ID NO: 1. In some embodiments, the variant NiV-G protein (also called variant NiV-G) contains an N-terminal methionine. In some embodiments, the variant NiV-G protein lacks amino acids 2-34 as compared to wild-type NiV-G set forth in SEQ ID NO: 1. In some embodiments, the NiV-G has the sequence set forth in SEQ ID NO:228.

In some embodiments, the variant NiV-G contains a heterologous cytoplasmic tail that is a cytoplasmic tail or a truncated portion thereof from a glycoprotein from CD63. In some embodiments, the heterologous cytoplasmic tail replaces at least a part of the native cytoplasmic tail of NiV-G (e.g. corresponding to amino acids 1-45 of SEQ ID NO:5). In some embodiments, the heterologous tail is a contiguous sequence of 5, 6, 7, 8, 9, 10, 11, 12, 13, 14, 15, 16, 17, 18, 19, 20, 21, 22, 23, 24, or 25 N-terminal amino acids of the native cytoplasmic tail of CD63. In some embodiments, the native cytoplasmic tail of CD63 is set forth in SEQ ID NOs: 200, 201, or 202. In some embodiments, the heterologous cytoplasmic tail is a truncated portion of the CD63 cytoplasmic tail set forth in any one of SEQ ID NOS: 200-205. In some embodiments, the variant NiV-G has a sequence in which the heterologous cytoplasmic tail set forth in any one of SEQ ID NOS: 200-205 is directly linked to the N-terminus of the sequence set forth in SEQ ID NO: 2. In some embodiments, the variant NiV-G has a sequence in which the heterologous cytoplasmic tail set forth in any one of SEQ ID NOS: 200-205 is directly linked to the N-terminus of the sequence set forth in SEQ ID NO: 3. In some embodiments, the variant NiV-G comprises a modified cytoplasmic tail which comprises a mutated cytoplasmic tail from a glycoprotein from the same Nipah virus. In some embodiments, the variant NiV-G contains a modified cytoplasmic tail in which at least a part of the native cytoplasmic tail (e.g. corresponding to amino acids 1-45 of SEQ ID NO:5) is a mutated portion thereof from a glycoprotein from Nipah Virus. In some embodiments, the cytoplasmic tail is a mutated portion of the Nipah virus cytoplasmic tail set forth in any one of SEQ ID NOS: 29-38. In some embodiments, it is understood that the truncated NiV-G cytoplasmic tail may include the sequence set forth in any one of SEQ ID NOS: 29-38 that lacks the N-terminal methionine. In some embodiments, the variant NiV-G has a sequence in which the cytoplasmic tail set forth in any one of SEQ ID NOS: 29-38 is directly linked to the N-terminus of the sequence set forth in SEQ ID NO: 2. In some embodiments, the variant NiV-G has a sequence in which the cytoplasmic tail set forth in any one of SEQ ID NOS: 29-38 is directly linked to the N-terminus of the sequence set forth in SEQ ID NO: 3.

In some embodiments, at least one G protein or the functionally active variant or biologically active portion thereof binds to Ephrin B2 or Ephrin B3. In some embodiments, the G protein is a variant G protein, such as a truncated G protein as described and retains binding to Ephrin B2 or B3. Reference to retaining binding to Ephrin B2 or B3 includes binding that is similar to the level or degree of binding of the corresponding wild-type G protein, such as set forth in SEQ ID NO: 1, 561, 562, 563, or 564, such as at least 50%, at least 60%, at least 70%, at least 80% or at least 90% of the binding of the wild-type G protein.

In some embodiments, also provided are any of the provided variant NiV-G proteins that are re-targeted compared to the native tropism of NiV-G. For instance, mutations in NiV-G that completely abrogate ephrinB2 and B3 binding, but that do not impact the association of this NiV-G with NiV-F, have been identified (Aguilar, et al. J Biol Chem. 2009; 284(3):1628-1635.; Weise et al. J Virol. 2010; 84(15):7634-764; Negrete et al., J Virol. 2007; 81(19):10804-10814; Negrete et al. PLoS Pathog. 2006; Guillaume et al., J. Virol 2006, 80 (15) 7546-7554). Thus, in provided aspects, a variant NiV-G protein provided herein may further contain a mutation in its extracellular domain to reduce or abrogate binding to Ephrin B2 and/B3. In some embodiments, the mutations can include one or more of mutations E501A, W504A, Q530A and E533A, with reference to numbering of wild-type NiV-G set forth in SEQ ID NO:5. In some embodiments, any of the provided variant NiV-G proteins may also be linked or fused to a binding molecule for targeted binding to a target molecule of interest. In some embodiments, the variant G protein is a fusion of a binding molecule with variant NiV-G, including a NiV-G with mutations to abrogate Ephrin B2 and/or Ephrin B3 binding. This could allow for altered G protein tropism allowing for targeting of other desired cell types that are not ephrinB2+ through the addition of the binding molecule directed against a different cell surface molecule.

In some embodiments, the paramyxovirus envelope attachment protein is a variant G protein that exhibits reduced binding for the native binding partner of a wild-type G protein. In some embodiments, the variant G protein or the biologically active portion thereof is a variant of wild-type NiV-G and exhibits reduced binding to one or both of the native binding partners Ephrin B2 or Ephrin B3. In some embodiments, the variant G-protein or the biologically active portion, such as a variant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations can improve transduction efficiency. In some embodiments, the mutations allow for specific targeting of other desired cell types that are not Ephrin B2 or Ephrin B3. In some embodiments, the mutations result in at least the partial inability to bind at least one natural receptor, such has reduced the binding to at least one of Ephrin B2 or Ephrin B3. In some embodiments, the mutations described herein interfere with natural receptor recognition.

In some embodiments, at least one G protein contains one or more amino acid substitutions in a residue that is involved in the interaction with one or both of Ephrin B2 and Ephrin B3. In some embodiments, the amino acid substitutions correspond to mutations E501A, W504A, Q530A and E533A with reference to numbering set forth in SEQ ID NO: 1. In some embodiments, at least one G protein is a variant G protein containing one or more amino acid substitutions selected from the group consisting of E501A, W504A, Q530A and E533A with reference to numbering set forth in SEQ ID NO:1. In some embodiments, at least one G protein is a variant G protein that contains one or more amino acid substitutions elected from the group consisting of E501A, W504A, Q530A and E533A with reference to SEQ ID NO: 1 and is a biologically active portion thereof containing an N-terminal truncation.

In particular embodiments, at least one G protein has the sequence of amino acids set forth in SEQ ID NO: 228, or is a functionally active variant thereof or a biologically active portion thereof that retains binding and/or fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 228 and retains fusogenic activity in conjunction with a variant NiV-F protein as described. In some embodiments, at least one G protein is a variant G protein that comprises the amino acid sequence of SEQ ID NO: 228.

In some embodiments, any of the provided lipid particles (lentiviral vectors) may also contain an F protein, such as a NiV-F protein, such as a full-length NiV-F protein or a biologically active portion thereof or a variant thereof. For instance, also provided herein are viral particles or viral-like particles, such as lentiviral particles or lentiviral-like particles, that are pseudotyped with any of the provided variant NiV-G proteins and a NiV-F protein, such as a full-length NiV-F protein or a biologically active portion or a variant thereof. Exemplary NiV-F proteins are further described in Section III.A.2.

Reference to retaining fusogenic activity includes activity of a lipid particle (e.g. lentiviral vector) containing at least two paramyxovirus envelope attachment protein and paramyxovirus fusion protein (e.g., F and G proteins) that is between at or about 10% and at or about 150% or more of the level or degree of binding of a reference lipid particle (e.g. lentiviral vector) that is similar, such as contains the same variant NiV-F, but that contains the corresponding wild-type G protein, such as set forth in SEQ ID NO: 1. For instance, a lipid particle (e.g. lentiviral vector) that retains fusogenic activity has at least or at least about 10% of the level or degree of fusogenic activity of the reference lipid particle that is similar (such as contains the same variant NiV-F) but that contains the corresponding wild-type G protein, such as at least or at least about 15% of the level or degree of fusogenic activity, at least or at least about 20% of the level or degree of fusogenic activity, at least or at least about 25% of the level or degree of fusogenic activity, at least or at least about 30% of the level or degree of fusogenic activity, at least or at least about 35% of the level or degree of fusogenic activity, at least or at least about 40% of the level or degree of fusogenic activity, at least or at least about 45% of the level or degree of fusogenic activity, at least or at least about 50% of the level or degree of fusogenic activity, at least or at least about 55% of the level or degree of fusogenic activity, at least or at least about 60% of the level or degree of fusogenic activity, at least or at least about 65% of the level or degree of fusogenic activity, at least or at least about 70% of the level or degree of fusogenic activity, at least or at least about 75% of the level or degree of fusogenic activity, at least or at least about 80% of the level or degree of fusogenic activity, at least or at least about 85% of the level or degree of fusogenic activity, at least or at least about 90% of the level or degree of fusogenic activity, at least or at least about 95% of the level or degree of fusogenic activity, at least or at least about 100% of the level or degree of fusogenic activity, or at least or at least about 120% of the level or degree of fusogenic activity.

Reference to retaining fusogenic activity includes activity of a lipid particle (e.g. lentiviral vector) containing at least two paramyxovirus envelope attachment protein and paramyxovirus fusion protein (e.g., F and G proteins) that is between at or about 10% and at or about 150% or more of the level or degree of binding of a reference lipid particle (e.g. lentiviral vector) that is similar, such as contains the same variant NiV-F, but that contains only one of the provided paramyxovirus envelope attachment proteins (e.g., G proteins). For instance, a lipid particle (e.g. lentiviral vector) that retains fusogenic activity has at least or at least about 10% of the level or degree of fusogenic activity of the reference lipid particle that is similar (such as contains the same variant NiV-F) but that contains only one of the provided paramyxovirus envelope attachment proteins, such as at least or at least about 15% of the level or degree of fusogenic activity, at least or at least about 20% of the level or degree of fusogenic activity, at least or at least about 25% of the level or degree of fusogenic activity, at least or at least about 30% of the level or degree of fusogenic activity, at least or at least about 35% of the level or degree of fusogenic activity, at least or at least about 40% of the level or degree of fusogenic activity, at least or at least about 45% of the level or degree of fusogenic activity, at least or at least about 50% of the level or degree of fusogenic activity, at least or at least about 55% of the level or degree of fusogenic activity, at least or at least about 60% of the level or degree of fusogenic activity, at least or at least about 65% of the level or degree of fusogenic activity, at least or at least about 70% of the level or degree of fusogenic activity, at least or at least about 75% of the level or degree of fusogenic activity, at least or at least about 80% of the level or degree of fusogenic activity, at least or at least about 85% of the level or degree of fusogenic activity, at least or at least about 90% of the level or degree of fusogenic activity, at least or at least about 95% of the level or degree of fusogenic activity, at least or at least about 100% of the level or degree of fusogenic activity, or at least or at least about 120% of the level or degree of fusogenic activity.

### B) Retargeted Attachment Proteins

In some embodiments, a paramyxovirus envelope attachment protein, such as a G protein (e.g., NiV-G), is further attached or linked to a binding domain that binds to a target molecule to comprise a retargeted attachment protein. For instance, provided in some aspects is a lipid particle that includes a targeted paramyxovirus envelope attachment proteins (e.g., a chimeric attachment G protein) containing any of the provided G proteins described above that is attached to a binding domain, in which the retargeted attachment protein (e.g., re-targeted G protein) is exposed on the surface of the targeted lipid particle (e.g. lentiviral vector). In some of any of the provided embodiments, the lipid particle comprises a retargeted attachment protein comprising (i) a paramyxovirus envelope attachment protein; and (ii) a targeting moiety directed to a first target molecule expressed on the surface of a target cell.

In some embodiments, the paramyxovirus envelope attachment protein, such as a G protein (e.g., NiV-G), is further attached or linked to targeting moiety, e.g., a binding domain or a binding agent, directed to a target molecule expressed on the surface of a target cell. The binding domain or binding agent can be any binding domain or binding agent described herein, e.g., in Section III.A.1.B. Accordingly, in some embodiments, the lipid particle comprises one or more retargeted attachment proteins, wherein each of the one or more retargeted attachment proteins independently comprise: (i) a paramyxovirus envelope attachment protein; and (ii) a targeting moiety directed to a target molecule expressed on the surface of a target cell. The targeting moiety can be a binding domain or binding agent, such as any binding domain or any binding agent described herein, e.g., in Section III.A.1.B.

In some embodiments, a paramyxovirus envelope attachment protein, such as a G protein (e.g., NiV-G), is further attached or linked in tandem with at least two binding domains that bind to a first and second target molecule respectively to comprise a retargeted attachment protein. For instance, provided in some aspects is a lipid particle that includes a targeted paramyxovirus envelope attachment proteins (e.g., a chimeric attachment G protein) containing any of the provided G proteins described above that is attached (e.g., operably fused in tandem) to a first and second binding domain, in which the retargeted attachment protein (e.g., re-targeted G protein) is exposed on the surface of the targeted lipid particle (e.g. lentiviral vector). In some of any of the provided embodiments, the lipid particle comprises a retargeted attachment protein comprising (i) a paramyxovirus envelope attachment protein; and (ii) a first targeting moiety directed to a first target molecule expressed on the surface of a target cell, and (iii) a second targeting moiety directed to a second target molecule expressed on the surface of a target cell.

In some embodiments, each of the one or more of the paramyxovirus envelope attachment proteins, such as a G protein (e.g., NiV-G), is further attached or linked in tandem to a first and a second targeting moiety, e.g., a first and second binding domain or a binding agent, directed to a first and second target molecule expressed on the surface of a target cell. The binding domains or binding agents can be individually selected from any binding domain or binding agent described herein, e.g., in Section III.A.1.B. Accordingly, in some embodiments, the lipid particle comprises one or more retargeted attachment proteins, wherein each of the one or more retargeted attachment proteins independently comprise: (i) a paramyxovirus envelope attachment protein; and (ii) a first targeting moiety directed to a first target molecule expressed on the surface of a target cell, and (iii) a second targeting moiety directed to a second target molecule expressed on the surface of a target cell. The targeting moieties can be a binding domain or binding agent, such as any binding domain or any binding agent described herein, e.g., in Section III.A.1.B.

In some embodiments, the envelope attachment protein is a retargeted attachment protein containing a henipavirus G protein or a biologically active portion thereof. In some embodiments, the envelope attachment proteins (e.g., G protein) may be retargeted by linkage to a targeting moiety, such as a binding molecule (e.g. antibody or antigen-binding fragment, e.g. sdAb or scFv) that binds to a target cell, such as a retargeted attachment protein. In some embodiments, the retargeted attachment protein and paramyxovirus fusion protein (e.g., G protein and a NiV-F protein provided herein) together exhibit fusogenic activity to a target cell, such as to deliver an exogenous agent or nucleic acid exogenous agent to the target cell.

In some embodiments, the targeting the target molecule does not activate or inhibit, induce a phenotype change (for example maturation and/or differentiation), induce proliferation, and/or induce apoptosis of said target cell.

In some embodiments, the paramyxovirus retargeted attachment protein is a targeted envelope protein containing a G protein provided herein. In some embodiments the paramyxovirus retargeted attachment protein comprises at least one envelope attachment proteins (e.g., G protein) that is any of those provided in Section III.A.1.A, including NiV-G proteins with cytoplasmic domain modifications, truncated NiV-G cytoplasmic tails, or modified NiV-G cytoplasmic tails.

In some of any embodiments, the retargeted attachment protein comprises (i) a paramyxovirus envelope attachment protein; and (ii) a targeting moiety directed to a first target molecule expressed on the surface of a target cell. In some of any embodiments, the retargeted attachment protein, comprises (i) a paramyxovirus envelope attachment protein; and (ii) a targeting moiety directed to a target molecule expressed on the surface of a target cell. In some embodiments, the targeting moiety is a binding domain, such as any of the binding domains or binding agents described herein in Section III.A.1.B, e.g., a T cell binding domain or an HSC binding domain. In some embodiments, the binding domain can be any agent that binds to a cell surface molecule on a target cells. In some embodiments, the binding domain can be an antibody or an antibody portion or fragment. In some embodiments, the binding domain is a single domain antibody (sdAb). In some embodiments, the binding domain is a single chain variable fragment (scFv). The binding domain can be linked directly or indirectly to the G protein. In particular embodiments, the binding domain is linked to the C-terminus (C-terminal amino acid) of the G protein or the biologically active portion thereof. The linkage can be via a peptide linker, such as a flexible peptide linker.

The retargeted attachment protein comprising a binding domain linked to a paramyxovirus envelope attachment protein may be modulated to have different binding strengths. For example, scFvs and antibodies with various binding strengths may be used to alter the fusion activity of the retargeted attachment proteins towards cells that display high or low amounts of the target antigen. For example DARPins with different affinities may be used to alter the fusion activity towards cells that display high or low amounts of the target antigen. Binding domains may also be modulated to target different regions on the target ligand, which will affect the fusion rate with cells displaying the target.

The binding domain may comprise a humanized antibody molecule, intact IgA, IgG, IgE or IgM antibody; bi- or multi-specific antibody (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. A targeting moiety can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs).

The binding domain may comprise a humanized antibody molecule, intact IgA, IgG, IgE or IgM antibody; bi- or multi-specific antibody (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. A targeting moiety can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody or a T cell receptor (TCRs). In some embodiments, the binding domain does not comprise a ligand, a cytokine, or a chemokine,

In some embodiments, the binding domain is a single chain molecule. In some embodiments, the binding domain is a single domain antibody. In some embodiments, the binding domain is a single chain variable fragment. In particular embodiments, the binding domain contains an antibody variable sequence (s) that is human or humanized.

In some embodiments, the binding domain is a single domain antibody. In some embodiments, the single domain antibody can be human or humanized. In some embodiments, the single domain antibody or portion thereof is naturally occurring. In some embodiments, the single domain antibody or portion thereof is synthetic.

In some embodiments, the single domain antibodies are antibodies whose complementary determining regions are part of a single domain polypeptide. In some embodiments, the single domain antibody is a heavy chain only antibody variable domain. In some embodiments, the single domain antibody does not include light chains.

In some embodiments, the heavy chain antibody devoid of light chains is referred to as VHH. In some embodiments, the single domain antibody antibodies have a molecular weight of 12-15 kDa. In some embodiments, the single domain antibody antibodies include camelid antibodies or shark antibodies. In some embodiments, the single domain antibody molecule is derived from antibodies raised in Camelidae species, for example in camel, llama, dromedary, alpaca, vicuna and guanaco. In some embodiments, the single domain antibody is referred to as immunoglobulin new antigen receptors (IgNARs) and is derived from cartilaginous fishes. In some embodiments, the single domain antibody is generated by splitting dimeric variable domains of human or mouse IgG into monomers and camelizing critical residues.

In some embodiments, the single domain antibody can be generated from display libraries, e.g., phage display libraries. In some embodiments, the display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the display library is generated comprising antibody fragments of a non-immunized camelid. In some embodiments, single domain antibodies a library of human single domain antibodies is synthetically generated by introducing diversity into one or more scaffolds.

In some embodiments, the binding domain is a single domain antibody (sdAb). In some embodiments, the binding domain is a single chain variable fragment (scFv). The binding domain can be linked directly or indirectly to the paramyxovirus envelope attachment protein, first paramyxovirus envelope attachment protein, and/or second paramyxovirus envelope attachment protein (e.g., G protein). In particular embodiments, the binding domain is linked to the C-terminus (C-terminal amino acid) of the G protein or the biologically active portion thereof. The linkage can be via a peptide linker, such as a flexible peptide linker.

In some embodiments, the C-terminus of the binding domain is attached to the C-terminus of the G protein or biologically active portion thereof. In some embodiments, the N-terminus of the binding domain is exposed on the exterior surface of the lipid bilayer. In some embodiments, the N-terminus of the binding domain binds to a cell surface molecule of a target cell. In some embodiments, the binding domain specifically binds to a cell surface molecule present on a target cell. In some embodiments, the cell surface molecule is a protein, glycan, lipid or low molecular weight molecule. In some embodiments, the binding domain is one of any binding domains as described above.

In some embodiments, a binding domain (e.g. sdAb or one of any binding domains as described herein) binds to a cell surface antigen of a cell. In some embodiments, a cell surface antigen is characteristic of one type of cell. In some embodiments, a cell surface antigen is characteristic of more than one type of cell.

In some embodiments, the cell surface molecule of a target cell is an antigen or portion thereof. In some embodiments, the single domain antibody or portion thereof is an antibody having a single monomeric domain antigen binding/recognition domain that is able to bind selectively to a specific antigen. In some embodiments, the single domain antibody binds an antigen present on a target cell.

Exemplary cells include polymorphonuclear cells (also known as PMN, PML, PMNL, or granulocytes), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, immune effector cells, lymphocytes, macrophages, dendritic cells, natural killer cells, T cells, cytotoxic T lymphocytes, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes.

In some embodiments, the target cell is a cell of a target tissue. The target tissue can include liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g. hepatocyte), or a cardiac cell (e.g. cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g. a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoietic stem cell (HSC), a liver cell or a fully differentiated cell. In some embodiments, the target cell is a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell). In some embodiments, the first and second target molecules are present on the same target cell. In some embodiments, the first and second target molecules are present on different cells.

In some embodiments, the binding domain (e.g. sdAb) variable domain binds a cell surface molecule or antigen. In some embodiments, the cell surface molecule is ASGR1, ASGR2, TM4SF5, CD8, CD4, or low density lipoprotein receptor (LDL-R). In some embodiments, the cell surface molecule is ASGR1. In some embodiments, the cell surface molecule is ASGR2. In some embodiments, the cell surface molecule is TM4SF5. In some embodiments, the cell surface molecule is CD8. In some embodiments, the cell surface molecule is CD4. In some embodiments, the cell surface molecule is LDL-R.

In some embodiments, the target cell is a hematopoietic lineage cell. Reference to a “hematopoietic cell” includes blood cells, both from the myeloid and the lymphoid lineage. In particular, the term “hematopoietic cell” includes both undifferentiated or poorly differentiated cells, such as hematopoietic stem cells and progenitor cells, and differentiated cells such as T lymphocytes, B lymphocytes, or dendritic cells. In some embodiments, the hematopoietic cells are hematopoietic stem cells (HSCs), CD34+ progenitor cells, in particular peripheral blood CD34+ cells, very early progenitor CD34+ cells, B-cell CD19+ progenitors, myeloid progenitor CD13+ cells, T lymphocytes, B lymphocytes, monocytes, dendritic cells, cancer B cells in particular B-cell chronic lymphocytic leukemia (BCLL) cells and marginal zone lymphoma (MZL) B cells, or thymocytes.

As known from the skilled person, many hematopoietic cells are produced from bone marrow hematopoietic stem cells.

In some embodiments, a hematopoietic cell is a hematopoietic stem cell (HSC), which are cells able to replenish all blood cell types and to self-renew. Hematopoietic stem cells may be in particular defined as cells that keep the levels of myeloid, T cells, and B cells at robustly detectable levels (typically more than 1% of peripheral blood cells) for 16 weeks when injected into the circulation of a recipient mouse with a depleted hematopoietic system (Schroeder (2010) Cell Stem Cell 6:203-207).

In some embodiments, the hematopoietic cell is a “CD34+ progenitor cell,” which is a heterogeneous cell population that includes a subpopulation of HSCs, pluripotent stem cells and cells in the early stages of lineage commitment. CD34+ progenitor cells continuously migrate to and from the bone marrow in normal adult animals. They can differentiate to produce all hematopoietic cell lineages found in the circulation. In some embodiments, the hematopoietic cell is a very early progenitor CD34+ cell which is a subgroup of CD34+ progenitor cells enriched from HSCs.

In some embodiments, the hematopoietic cell is a “peripheral blood CD34+ cell”, which is a CD34+ cell present in the blood.

In some embodiments, the hematopoietic cell is a B cell CD19+ progenitor, which is a population of B-lineage cells that express cell surface CD10, CD34, and CD19.

In some embodiments, the hematopoietic cell is a myeloid progenitor CD13+ cells, which is a population of myeloid lineage cells that express cell surface CD34 and CD13, and in some cases, also CD33.

In some embodiments, the target cell is selected from the group consisting of myeloid-lymphoid balanced hematopoietic lineage cells, myeloid-biased hematopoietic lineage cells, lymphoid-biased hematopoietic lineage cells, a platelet-biased hematopoietic lineage cells, a platelet-myeloid-biased hematopoietic lineage cells, a long-term repopulating hematopoietic lineage cells, an intermediate-term repopulating hematopoietic lineage cells, or a short-term repopulating hematopoietic lineage cells. In some embodiments, the target cell is selected from monocytes, macrophages, neutrophils, basophils, eosinophils, erythrocytes, megakaryocytes and platelets. In some embodiments, the target cell is selected from T cells, B cells, natural killer (NK) cells and innate lymphoid cells.

In some embodiments the target cell is an effector cell, e.g., a cell of the immune system that expresses one or more Fc receptors and mediates one or more effector functions. In some embodiments, a target cell may include one or more of a monocyte, macrophage, neutrophil, dendritic cell, eosinophil, mast cell, platelet, large granular lymphocyte, Langerhans' cell, natural killer (NK) cell, T lymphocyte (e.g., T cell), a Gamma delta T cell, B lymphocyte (e.g., B cell) and may be from any organism including humans, mice, rats, rabbits, and monkeys.

In some embodiment, the hematopoietic cell is a T cell. In some embodiments, the T cell is a naïve T cell. In some embodiments, the T cell is a memory T cell.

In some embodiments, the hematopoietic cell is a B cell. In some embodiments, the target cell is a resting B cell, such as a naive or a memory B cell. In some embodiments, the target cell is a cancer B cell, such as a B-cell chronic lymphocytic leukemia (BCLL) cell or a marginal zone lymphoma (MZL) B cell.

In some embodiments, the target cell is a thymocyte. In some embodiments, the target cell is a natural killer (NK) cell. In some embodiments, the thymocyte expresses CD4 or CD8. In some embodiments, the thymocyte does not express CD4 or CD8. In some embodiments, the natural killer (NK) cell is a cell that expresses CD56.

In some embodiments, the target cell is a CD3+ T cell, a CD4+ T cell, or a CD8+ T cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, or a B cell.

In some embodiments, the binding domain (e.g. sdAb) variable domain binds a cell surface molecule or antigen. In some embodiments, the cell surface molecule is ASGR1, ASGR2, TM4SF5, CD3, CD8, CD4, CD7, or low density lipoprotein receptor (LDL-R). In some embodiments, the cell surface molecule is ASGR1. In some embodiments, the cell surface molecule is ASGR2. In some embodiments, the cell surface molecule is TM4SF5. In some embodiments, the cell surface molecule is CD3. In some embodiments, the cell surface molecule is CD8. In some embodiments, the cell surface molecule is CD4. In some embodiments, the cell surface molecule is LDL-R. In some embodiments, the cell surface molecule is ASCT2, CD105, CD110, CD117, CD133, CD146, CD164, CD34, CD46, CD49f, CD90, EPCR, or ITGA3.

In some embodiments, the retargeted attachment protein comprises the paramyxovirus envelope attachment protein (e.g., G protein or functionally active variant or biologically active portion thereof) linked directly to the binding domain and/or variable domain thereof. In some embodiments, the targeted envelope protein is a fusion protein that has the following structure: (N′-single domain antibody-C′)-(C′-G protein-N′).

In some embodiments, the retargeted attachment protein comprises the paramyxovirus envelope attachment protein (e.g., G protein or functionally active variant or biologically active portion thereof) linked indirectly via a linker to the binding domain and/or variable domain thereof. In some embodiments, the linker is a peptide linker. In some embodiments, the linker is a chemical linker.

In some embodiments, the linker is a peptide linker and the targeted envelope protein is a fusion protein containing the paramyxovirus envelope attachment protein (e.g., G protein or functionally active variant or biologically active portion thereof) linked via a peptide linker to the binding molecule variable domain (e.g. antibody or antigen-binding fragment, e.g. sdAb or scFv variable domain). In some embodiments, the targeted envelope protein is a fusion protein that has the following structure: (N′-single domain antibody-C′)-Linker-(C′-G protein-N′).

In some embodiments, the peptide linker is up to 65 amino acids in length. In some embodiments, the peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids. In some embodiments, the peptide linker is a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 amino acids in length.

In particular embodiments, the linker is a flexible peptide linker. In some such embodiments, the linker is 1-20 amino acids, such as 1-20 amino acids predominantly composed of glycine. In some embodiments, the linker is 1-20 amino acids, such as 1-20 amino acids predominantly composed of glycine and serine. In some embodiments, the linker is a flexible peptide linker containing amino acids Glycine and Serine, referred to as GS-linkers. In some embodiments, the peptide linker includes the sequences GS, GGS, GGGGS, GGGGGS or combinations thereof. In some embodiments, the polypeptide linker has the sequence (GGS)n, wherein n is 1 to 10. In some embodiments, the polypeptide linker has the sequence (GGGGS)n, wherein n is 1 to 10. In some embodiments, the polypeptide linker has the sequence (GGGGGS)n, wherein n is 1 to 6. In some embodiments, the polypeptide linker has or comprises the amino acid sequence of SEQ ID NO: 405 (GGGGSGGGGSGGGGS). In some embodiments, the retargeted attachment protein comprising a binding domain, a first binding domain, and/or a second binding domain linked to at least one paramyxovirus envelope attachment may comprise an engineered binding domain, such as an artificially generated binding domain. The binding domain may comprise a nanobody, a DARPin, an Aptamer, an Affimer, an Affibody, a Knottin, an Avimer, a Monobody, an Anticalin, a Fynomer. Any engineered binding domain known in art and suitable for the present invention can be used, for example any such binding domain described in Olaleye et al. Biomolecules. 2021 December; 11(12): 1791.

### I. T Cell Binding Domains

In some embodiments, the lipid particles disclosed herein comprise one or more retargeted attachment proteins, each independently comprising (i) a paramyxovirus envelope attachment protein; and (ii) a targeting moiety directed to a target molecule expressed on the surface of a target cell. In some embodiments, the targeting moiety is a T cell binding domain, e.g., a T cell binding agent, such as any of those disclosed herein. In some embodiments, the retargeted attachment protein comprises a targeting moiety. In some embodiments, the retargeted attachment protein comprises a targeting moiety that is a T cell binding domain.

The lipid particles disclosed herein include, in some embodiments, one or more T cell binding domains (e.g., a T cell binding agent) that target the viral vector to a T cell. In some embodiments, the T cell binding agent binds to a molecule expressed on the surface of the T cell. The cell surface molecule may be a receptor, coreceptor, or a GPI-anchored protein. In some embodiments, the T cell binding agent binds CD3, CD4 or CD8.

In particular embodiments, a T cell binding agent may be fused to or incorporated in a protein fusogen or attachment protein, thereby retargeting the lipid particle to a T cell. In some embodiments, for re-targeting the T cell binding agent is fused to a protein fusogen or envelope attachment protein that is mutated to reduce binding for the native binding partner of the fusogen or viral envelope protein. In some embodiments, the fusogen is or contains a mutant G protein or a biologically active portion thereof that is a mutant of wild-type NiV-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi:10.1038/nbt942, Molecular Therapy vol. 16 no. 8, 1427-1436 August 2008, doi:10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens may be re-targeted by covalently conjugating a T cell binding agent to the attachment protein. In some embodiments, the fusogen and T cell binding agent are covalently conjugated by expression of a chimeric protein comprising the fusogen linked to the T cell binding agent (e.g., retargeted attachment protein). The T cell binding agent can include any targeting protein able to confer specific binding to a target molecule expressed on the surface of a T cell. In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, the T cell binding agent is an antibody or antigen binding fragment thereof. In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nml192). In some embodiments, altered and non-altered fusogens may be displayed on the same retroviral vector or VLP (doi: 10.1016/j.biomaterials.2014.01.051).

In some embodiments, a single-chain variable fragment (scFv) can be conjugated to fusogens to redirect fusion activity towards T cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards T cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, a single domain antibody (e.g., a VHH) can be conjugated to fusogens to redirect fusion activity towards T cells that display the sdAb binding target. In some embodiments, receptor ligands and antigens can be conjugated to fusogens to redirect fusion activity towards T cells that display the target receptor (DOI: 10.1089/hgtb.2012.054, DOI: 10.1128/JVI.76.7.3558-3563.2002).

### a) CD3 Binding Agents

In some embodiments, the lipid particles disclosed herein comprise one or more retargeted attachment proteins, each independently comprising (i) a paramyxovirus envelope attachment protein; and (ii) a targeting moiety directed to a target molecule expressed on the surface of a target cell, wherein the target molecule is CD3. In some embodiments, the targeting moiety is a CD3 binding domain, e.g., a CD3 binding agent, such as any of those disclosed herein.

In some embodiments, CD3 binding domain may be fused to or incorporated in a protein fusogen or lipid particle envelope attachment protein (e.g., a retargeted attachment protein). In some embodiments, the CD3 binding domain may be incorporated into the co-display protein. In some embodiments, a CD3 binding domain may be incorporated into the viral envelope via fusion with a transmembrane domain. In some embodiments, the CD3 binding domain targets the lipid particle to a T cell.

The lipid particles disclosed herein include, in some embodiments, one or more CD3 binding agents. For example, a CD3 binding agent may be fused to or incorporated in a retargeted attachment protein. In another embodiment, a CD3 binding agent may be incorporated into the lipid particle envelope via fusion with a transmembrane domain.

Exemplary CD3 binding agents include antibodies and fragments thereof (e.g., scFv, VHH) that bind to CD3. Such antibodies may be derived from any species, and may be for example, mouse, rabbit, human, humanized, or camelid antibodies.

Exemplary antibodies include OKT3, CRIS-7, I2C, blinatumomab, catumaxomab, muromonab-CD3, A-319, AFM11, AMG 199, AMG 211, AMG 424, AMG 427, AMG 562, AMG 564, APV0436, CC-93269, ERY974, GBR1302, GEM333, GEM2PSCA, GNC-035, HPN424, IGM-2323, JNJ-63709178, JNJ-63898081, JNJ-75348780, JNJ-78306358, M701, M802, MGD007, MOR209/ES414, PF-06671008, REGN5459, RO7283420, SAR442257, SAR443216, TNB-383B, TNB-486, TNB-585, Y150, acapatamab, cevostamab, cibisatamab, duvortuxizumab, eluvixtamab, emerfetamab, etevritamab, glofitamab, gresonitamab, obrindatamab, pavurutamab, plamotamab, solitomab, tarlatamab, tepoditamab, tidutamab, vibecotamab, vixtimotamab, alnuctamab, dafsolimab setaritox, pacanalotamab, pasotuxizumab, runimotamab, nivatrotamab, elranatamab, ertumaxomab, flotetuzumab, odronextamab, talquetamab, teclistamab, visilizumab, epcoritamab, otelixizumab, 3F8BiAb, CCW702, DKTK CC-1, EMB-06, GEN1044, GEN1047, GTB-3550, HPN217, IMC-C103C, NVG-111, REGN4018, REGN4336, REGN5458, A-2019, A-337, ABP-100, AFM15, AFM21, AMG 701, APV0425, CLN-049, Dow2, EM801, Ektomab, FBTA05, GBR1342, GBR1372, GSK3537142, HBM7020, HLX31, IGM-2644, MG1122, MGD015, ND003, ND007, PF-07062119, RO7293583, STA551, TT19, ZW38; and anti-CD3 antibodies disclosed in U.S. Pat. Nos. 4,361,549, 7,728,114, 9,657,102, 9,587,021, and 11007267; US Patent Application Nos. US20120269826, US20180057597, and US20180112000; and PCT Application Nos. WO2005118635, WO2011050106, WO2012162067, WO2014047231, WO2016116626, WO2016180721, and WO2016204966. Other exemplary binding agents include designed ankyrin repeat proteins (DARPins) and binding agents based on fibronectin type III (Fn3) scaffolds.

In some embodiments, the CD3 binding agent comprises a heavy chain variable (VH) region comprising a CDR-H1, a CDRH-2, and a CDR-H3 comprising the amino acid sequence set forth in SEQ ID NO:474, 475, and 476 respectively; and a light chain variable region comprising a CDR-L1, a CDR-L2, and a CDR-L3 comprising the amino acid sequence set forth in SEQ ID NO:477, 478, and 479, respectively. In some embodiments, the CD3 binding agent comprises a VH region comprising an amino acid sequence having at least about 90% sequence identity to the amino acid sequence set forth in SEQ ID NO:480, and a VL region comprising an amino acid sequence having at least about 90% sequence identity to the amino acid sequence set forth in SEQ ID NO:481. In some embodiments, the CD3 binding agent comprises a VH region comprising the amino acid sequence set forth in SEQ ID NO:480, and a VL region comprising the amino acid sequence set forth in SEQ ID NO:481. In some embodiments, the CD3 binding agent is an scFv. In some embodiments, the CD3 binding agent comprises the amino acid sequence set forth in SEQ ID NO:482. In some embodiments, the CD3 binding agent comprises the amino acid sequence set forth in SEQ ID NO:802. In some embodiments, the CD3 binding agent is OKT3.

In some embodiments, the CD3 binding agent is activating (e.g., the CD3 binding agent activates T cells). In some embodiments, the CD3 binding agent is non-activating (e.g., it does not activate T cells).

In some embodiments, a CD3 binding agent comprises a humanized antibody molecule, intact IgA, IgG, IgE or IgM antibody; bi- or multi-specific antibody (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); camelid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In some embodiments, the CD3 binding agent is a peptide. In some embodiments, the CD3 binding agent is an antibody, such as a single-chain variable fragment (scFv). In some embodiments, the CD3 binding agent is an antibody, such as a single domain antibody. In some embodiments, the antibody can be human or humanized. In some embodiments, the CD3 binding agent is a VHH. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD3 binding agent is attached to the C-terminus of the G protein (e.g., fusogen) or biologically active portion thereof. In some embodiments, the N-terminus of the CD3 binding agent is exposed on the exterior surface of the lipid bilayer.

In some embodiments, the CD3 binding agent is the only surface displayed non-viral sequence of the viral vector. In some embodiments, the CD3 binding agent is the only membrane bound non-viral sequence of the viral vector. In some embodiments, the viral vector does not contain a molecule that engages or stimulates T cells other than the CD3 binding agent. In some embodiments, the viral vector contains a non-activating CD3 binding agent.

In some embodiments, viral vectors may display CD3 binding agents that are not conjugated to protein fusogens in order to redirect the fusion activity towards a cell that is bound by the targeting moiety, or to affect homing.

### b) CD4 Binding Agents

In some embodiments, the lipid particles disclosed herein comprise one or more retargeted attachment proteins, each independently comprising (i) a paramyxovirus envelope attachment protein; and (ii) a targeting moiety directed to a target molecule expressed on the surface of a target cell, wherein the target molecule is CD4. In some embodiments, the targeting moiety is a CD4 binding domain, e.g., a CD4 binding agent, such as any of those disclosed herein.

The lipid particles disclosed herein include, in some embodiments, one or more CD4 binding agents. For example, a CD4 binding agent may be fused to or incorporated in a protein fusogen or attachment protein. In another embodiment, a CD4 binding agent may be incorporated into the lipid particle envelope via fusion with a transmembrane domain.

In some of any of the provided embodiments, the CD4 binding agent is exposed on the surface of the lipid particle. In some embodiments, the CD4 binding agent is fused to a transmembrane domain incorporated in the lipid particle envelope.

In some embodiments, CD4 binding domain may be fused to or incorporated in a protein fusogen or lipid particle envelope attachment protein (e.g., a retargeted attachment protein). In some embodiments, the CD4 binding domain may be incorporated into the co-display protein. In some embodiments, a CD4 binding domain may be incorporated into the viral envelope via fusion with a transmembrane domain. In some embodiments, the CD3 binding domain targets the lipid particle to a T cell.

Exemplary CD4 binding agents include antibodies and fragments thereof (e.g., scFv, VHH) that bind to CD4. Such antibodies may be derived from any species, and may be for example, mouse, rabbit, human, humanized, or camelid antibodies. Exemplary antibodies include ibalizumab, zanolimumab, tregalizumab, priliximab, cedelizumab, clenoliximab, keliximab, and anti-CD4 antibodies disclosed in WO2002102853, WO2004083247, WO2004067554, WO2007109052, WO2008134046, WO2010074266, WO2012113348, WO2013188870, WO2017104735, WO2018035001, WO2018170096, WO2019203497, WO2019236684, WO2020228824, U.S. Pat. Nos. 5,871,732, 7,338,658, 7,722,873, 8,399,621, 8,911,728, 9,005,963, 9,587,022, 9,745,552, U.S. provisional application No. 63/326,269, U.S. provisional application No. 63/341,681; as well as antibodies B486A1, RPA-T4, CE9.1 (Novus Biologicals); GK1.5, RM4-5, RPA-T4, OKT4, 4SM95, S3.5, N1UGO (ThermoFisher); GTX50984, ST0488, 10B5, EP204 (GeneTex); GK1.3, 5A8, 10C12, W3/25, 8A5, 13B8.2, 6G5 (Absolute Antibody); VIT4, M-T466, M-T321, REA623, (Miltenyi); MEM115, MT310 (Enzo Life Sciences); H129.19, 5B4, 6A17, 18-46, A-1, C-1, OX68 (Santa Cruz); EP204, D2E6M (Cell Signaling Technology). Other exemplary binding agents include designed ankyrin repeat proteins (DARPins) (e.g., the anti-CD4 DARPin disclosed in WO2017182585) and binding agents based on fibronectin type III (Fn3) scaffolds. Each of U.S. Pat. No. 9,005,963, U.S. provisional application No. 63/326,269, and U.S. provisional application No. 63/341,681 is incorporated by reference herein in its entirety.

In some embodiments, protein fusogens or attachment proteins may be re-targeted by mutating amino acid residues in a fusion protein or a targeting protein (e.g. the hemagglutinin (H) protein or G protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type NiV-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi:10.1038/nbt942, Molecular Therapy vol. 16 no. 8, 1427-1436 August 2008, doi:10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens may be re-targeted by covalently conjugating a CD4 binding agent to the fusion protein or attachment protein (e.g. retargeted attachment protein). In some embodiments, the fusogen and CD4 binding agent are covalently conjugated by expression of a chimeric protein comprising the fusogen linked to the CD4 binding agent (e.g. retargeted attachment protein). In some embodiments, a single-chain variable fragment (scFv) can be conjugated to fusogens to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a CD4 binding agent to the fusion protein or targeting protein (e.g. retargeted attachment protein)). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nml192). In some embodiments, altered and non-altered fusogens may be displayed on the same retroviral vector or VLP (doi: 10.1016/j.biomaterials.2014.01.051).

In some embodiments, a CD4 binding agent comprises a humanized antibody molecule, intact IgA, IgG, IgE or IgM antibody; bi- or multi-specific antibody (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); camelid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In some embodiments, the CD4 binding agent is a peptide. In some embodiments, the CD4 binding agent is an antibody, such as a single-chain variable fragment (scFv). In some embodiments, the CD4 binding agent is an antibody, such as a single domain antibody. In some embodiments, the antibody can be human or humanized. In some embodiments, the CD4 binding agent is a VHH. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD4 binding agent is attached to the C-terminus of the G protein (e.g., fusogen) or biologically active portion thereof. In some embodiments, the N-terminus of the CD4 binding agent is exposed on the exterior surface of the lipid bilayer.

In some embodiments, the CD4 binding agent is the only surface displayed non-viral sequence of the lipid particle. In some embodiments, the CD4 binding agent is the only membrane bound non-viral sequence of the lipid particle. In some embodiments, the lipid particle does not contain a molecule that engages or stimulates T cells other than the CD4 binding agent.

In some embodiments, lipid particles may display CD4 binding agents that are not conjugated to protein fusogens in order to redirect the fusion activity towards a cell that is bound by the targeting moiety, or to affect homing.

In some embodiments, a protein fusogen derived from a virus or organism that do not infect humans does not have a natural fusion targets in patients, and thus has high specificity.

In some of any of the provided embodiments, the CD4 binding agent is an anti-CD4 antibody or an antigen-binding fragment. In some of any of the provided embodiments, the anti-CD4 antibody or antigen-binding fragment is mouse, rabbit, human, or humanized. In some embodiments, the antigen-binding fragment is a single chain variable fragment (scFv). In some embodiments, the antigen-binding fragment is an anti-CD4 scFv.

In some embodiments, the anti-CD4 scFv comprises a CDR-H1, a CDR-H2, and a CDR-H3 comprising the amino acid sequence set forth in SEQ ID NO: 260, 261, and 262, respectively. In some embodiments, the anti-CD4 scFv comprises a CDR-L1, a CDR-L2, and a CDR-L3 comprising the amino acid sequence set forth in SEQ ID NO: 263, 264, and 265, respectively. In some embodiments, the anti-CD4 scFv comprises a CDR-H1, a CDR-H2, and a CDR-H3 comprising the amino acid sequence set forth in SEQ ID NO: 260, 261, and 262, respectively; and a CDR-L1, a CDR-L2, and a CDR-L3 comprising the amino acid sequence set forth in SEQ ID NO: 263, 264, and 265, respectively. In some embodiments, the anti-CD4 scFv comprises a CDR-H1, a CDR-H2, and a CDR-H3 comprising the amino acid sequence set forth in SEQ ID NO: 266, 267, and 268, respectively. In some embodiments, the anti-CD4 scFv comprises a CDR-L1, a CDR-L2, and a CDR-L3 comprising the amino acid sequence set forth in SEQ ID NO: 269, 270, and 265, respectively. In some embodiments, the anti-CD4 scFv comprises a CDR-H1, a CDR-H2, and a CDR-H3 comprising the amino acid sequence set forth in SEQ ID NO: 266, 267, and 268, respectively; and a CDR-L1, a CDR-L2, and a CDR-L3 comprising the amino acid sequence set forth in SEQ ID NO: 269, 270, and 265, respectively. In some embodiments, the anti-CD4 scFv comprises a CDR-H1, a CDR-H2, and a CDR-H3 comprising the amino acid sequence set forth in SEQ ID NO: 271, 272, and 268, respectively. In some embodiments, the anti-CD4 scFv comprises a CDR-L1, a CDR-L2, and a CDR-L3 comprising the amino acid sequence set forth in SEQ ID NO: 269, 270, and 265, respectively. In some embodiments, the anti-CD4 scFv comprises a CDR-H1, a CDR-H2, and a CDR-H3 comprising the amino acid sequence set forth in SEQ ID NO: 271, 272, and 268, respectively; and a CDR-L1, a CDR-L2, and a CDR-L3 comprising the amino acid sequence set forth in SEQ ID NO: 269, 270, and 265, respectively. In some embodiments, the anti-CD4 scFv comprises a heavy chain variable region (VH) comprising the amino acid sequence set forth in SEQ ID NO:273. In some embodiments, the anti-CD4 scFv comprises a light chain variable region (VL) comprising the amino acid sequence set forth in SEQ ID NO:274. In some embodiments, the anti-CD4 scFv comprises a heavy chain variable region (VH) comprising the amino acid sequence set forth in SEQ ID NO:273; and a light chain variable region (VL) comprising the amino acid sequence set forth in SEQ ID NO:274. In some embodiments, the VH and VL are joined by a linker. In some embodiments, the linker comprises the amino acid sequence set forth in SEQ ID NO:275. In some embodiments, the anti-CD4 scFv comprises the amino acid sequence set forth in SEQ ID NO:276.

In some embodiments, the anti-CD4 scFv comprises a CDR-H1, a CDR-H2, and a CDR-H3 comprising the amino acid sequence set forth in SEQ ID NO: 277, 278, and 279, respectively. In some embodiments, the anti-CD4 scFv comprises a CDR-L1, a CDR-L2, and a CDR-L3 comprising the amino acid sequence set forth in SEQ ID NO: 280, 281, and 282, respectively. In some embodiments, the anti-CD4 scFv comprises a CDR-H1, a CDR-H2, and a CDR-H3 comprising the amino acid sequence set forth in SEQ ID NO: 277, 278, and 279, respectively; and a CDR-L1, a CDR-L2, and a CDR-L3 comprising the amino acid sequence set forth in SEQ ID NO: 280, 281, and 282, respectively. In some embodiments, the anti-CD4 scFv comprises a CDR-H1, a CDR-H2, and a CDR-H3 comprising the amino acid sequence set forth in SEQ ID NO: 283, 284, and 285, respectively. In some embodiments, the anti-CD4 scFv comprises a CDR-L1, a CDR-L2, and a CDR-L3 comprising the amino acid sequence set forth in SEQ ID NO: 286, 287, and 288, respectively. In some embodiments, the anti-CD4 scFv comprises a CDR-H1, a CDR-H2, and a CDR-H3 comprising the amino acid sequence set forth in SEQ ID NO: 283, 284, and 285, respectively; and a CDR-L1, a CDR-L2, and a CDR-L3 comprising the amino acid sequence set forth in SEQ ID NO: 286, 287, and 288, respectively. In some embodiments, the anti-CD4 scFv comprises a CDR-H1, a CDR-H2, and a CDR-H3 comprising the amino acid sequence set forth in SEQ ID NO: 289, 290, and 285, respectively. In some embodiments, the anti-CD4 scFv comprises a CDR-L1, a CDR-L2, and a CDR-L3 comprising the amino acid sequence set forth in SEQ ID NO: 286, 287, and 282, respectively. In some embodiments, the anti-CD4 scFv comprises a CDR-H1, a CDR-H2, and a CDR-H3 comprising the amino acid sequence set forth in SEQ ID NO: 289, 290, and 285, respectively; and a CDR-L1, a CDR-L2, and a CDR-L3 comprising the amino acid sequence set forth in SEQ ID NO: 286, 287, and 282, respectively. In some embodiments, the anti-CD4 scFv comprises a heavy chain variable region (VH) comprising the amino acid sequence set forth in SEQ ID NO:291. In some embodiments, the anti-CD4 scFv comprises a light chain variable region (VL) comprising the amino acid sequence set forth in SEQ ID NO:292. In some embodiments, the anti-CD4 scFv comprises a heavy chain variable region (VH) comprising the amino acid sequence set forth in SEQ ID NO:291; and a light chain variable region (VL) comprising the amino acid sequence set forth in SEQ ID NO:292. In some embodiments, the VH and VL are joined by a linker. In some embodiments, the linker comprises the amino acid sequence set forth in SEQ ID NO:275. In some embodiments, the anti-CD4 scFv comprises the amino acid sequence set forth in SEQ ID NO:293.

In some embodiments, the anti-CD4 scFv comprises a CDR-H1, a CDR-H2, and a CDR-H3 comprising the amino acid sequence set forth in SEQ ID NO: 294, 295, and 296, respectively. In some embodiments, the anti-CD4 scFv comprises a CDR-L1, a CDR-L2, and a CDR-L3 comprising the amino acid sequence set forth in SEQ ID NO: 297, 298, and 299, respectively. In some embodiments, the anti-CD4 scFv comprises a CDR-H1, a CDR-H2, and a CDR-H3 comprising the amino acid sequence set forth in SEQ ID NO: 294, 295, and 296, respectively; and a CDR-L1, a CDR-L2, and a CDR-L3 comprising the amino acid sequence set forth in SEQ ID NO: 297, 298, and 299, respectively. In some embodiments, the anti-CD4 scFv comprises a CDR-H1, a CDR-H2, and a CDR-H3 comprising the amino acid sequence set forth in SEQ ID NO: 300, 301, 302, respectively. In some embodiments, the anti-CD4 scFv comprises a CDR-L1, a CDR-L2, and a CDR-L3 comprising the amino acid sequence set forth in SEQ ID NO: 303, 304, and 299, respectively. In some embodiments, the anti-CD4 scFv comprises a CDR-H1, a CDR-H2, and a CDR-H3 comprising the amino acid sequence set forth in SEQ ID NO: 300, 301, 302, respectively; and a CDR-L1, a CDR-L2, and a CDR-L3 comprising the amino acid sequence set forth in SEQ ID NO: 303, 304, and 299, respectively. In some embodiments, the anti-CD4 scFv comprises a CDR-H1, a CDR-H2, and a CDR-H3 comprising the amino acid sequence set forth in SEQ ID NO: 305, 306, 306, respectively. In some embodiments, the anti-CD4 scFv comprises a CDR-L1, a CDR-L2, and a CDR-L3 comprising the amino acid sequence set forth in SEQ ID NO: 303, 304, and 299, respectively. In some embodiments, the anti-CD4 scFv comprises a CDR-H1, a CDR-H2, and a CDR-H3 comprising the amino acid sequence set forth in SEQ ID NO: 305, 306, 302, respectively; and a CDR-L1, a CDR-L2, and a CDR-L3 comprising the amino acid sequence set forth in SEQ ID NO: 224, 225, and 172, respectively. In some embodiments, the anti-CD4 scFv comprises a heavy chain variable region (VH) comprising the amino acid sequence set forth in SEQ ID NO:307. In some embodiments, the anti-CD4 scFv comprises a light chain variable region (VL) comprising the amino acid sequence set forth in SEQ ID NO:308. In some embodiments, the anti-CD4 scFv comprises a heavy chain variable region (VH) comprising the amino acid sequence set forth in SEQ ID NO:307; and a light chain variable region (VL) comprising the amino acid sequence set forth in SEQ ID NO:308. In some embodiments, the VH and VL are joined by a linker. In some embodiments, the linker comprises the amino acid sequence set forth in SEQ ID NO:275. In some embodiments, the anti-CD4 scFv comprises the amino acid sequence set forth in SEQ ID NO:309.

In some embodiments, the anti-CD4 scFv comprises a CDR-H1, a CDR-H2, and a CDR-H3 comprising the amino acid sequence set forth in SEQ ID NO: 310, 311, and 312, respectively. In some embodiments, the anti-CD4 scFv comprises a CDR-L1, a CDR-L2, and a CDR-L3 comprising the amino acid sequence set forth in SEQ ID NO: 313, 314, and 315, respectively. In some embodiments, the anti-CD4 scFv comprises a CDR-H1, a CDR-H2, and a CDR-H3 comprising the amino acid sequence set forth in SEQ ID NO: 310, 311, and 312, respectively; and a CDR-L1, a CDR-L2, and a CDR-L3 comprising the amino acid sequence set forth in SEQ ID NO: 313, 314, and 315, respectively. In some embodiments, the anti-CD4 scFv comprises a CDR-H1, a CDR-H2, and a CDR-H3 comprising the amino acid sequence set forth in SEQ ID NO: 316, 317, 318, respectively. In some embodiments, the anti-CD4 scFv comprises a CDR-L1, a CDR-L2, and a CDR-L3 comprising the amino acid sequence set forth in SEQ ID NO: 319, 320, and 315, respectively. In some embodiments, the anti-CD4 scFv comprises a CDR-H1, a CDR-H2, and a CDR-H3 comprising the amino acid sequence set forth in SEQ ID NO: 316, 317, 318, respectively; and a CDR-L1, a CDR-L2, and a CDR-L3 comprising the amino acid sequence set forth in SEQ ID NO: 319, 320, and 315, respectively. In some embodiments, the anti-CD4 scFv comprises a CDR-H1, a CDR-H2, and a CDR-H3 comprising the amino acid sequence set forth in SEQ ID NO: 321, 322, 318, respectively. In some embodiments, the anti-CD4 scFv comprises a CDR-L1, a CDR-L2, and a CDR-L3 comprising the amino acid sequence set forth in SEQ ID NO: 319, 321, and 315, respectively. In some embodiments, the anti-CD4 scFv comprises a CDR-H1, a CDR-H2, and a CDR-H3 comprising the amino acid sequence set forth in SEQ ID NO: 321, 322, 318, respectively; and a CDR-L1, a CDR-L2, and a CDR-L3 comprising the amino acid sequence set forth in SEQ ID NO: 319, 320, and 323, respectively. In some embodiments, the anti-CD4 scFv comprises a heavy chain variable region (VH) comprising the amino acid sequence set forth in SEQ ID NO:323. In some embodiments, the anti-CD4 scFv comprises a light chain variable region (VL) comprising the amino acid sequence set forth in SEQ ID NO:324. In some embodiments, the anti-CD4 scFv comprises a heavy chain variable region (VH) comprising the amino acid sequence set forth in SEQ ID NO:323; and a light chain variable region (VL) comprising the amino acid sequence set forth in SEQ ID NO:324. In some embodiments, the VH and VL are joined by a linker. In some embodiments, the linker comprises the amino acid sequence set forth in SEQ ID NO:275. In some embodiments, the anti-CD4 scFv comprises the amino acid sequence set forth in SEQ ID NO:325.

In some embodiments, the anti-CD4 scFv comprises a CDR-H1, a CDR-H2, and a CDR-H3 comprising the amino acid sequence set forth in SEQ ID NO: 326, 327, and 328, respectively. In some embodiments, the anti-CD4 scFv comprises a CDR-L1, a CDR-L2, and a CDR-L3 comprising the amino acid sequence set forth in SEQ ID NO: 329, 330, and 331, respectively. In some embodiments, the anti-CD4 scFv comprises a CDR-H1, a CDR-H2, and a CDR-H3 comprising the amino acid sequence set forth in SEQ ID NO: 326, 327, and 328, respectively; and a CDR-L1, a CDR-L2, and a CDR-L3 comprising the amino acid sequence set forth in SEQ ID NO: 329, 330, and 331, respectively. In some embodiments, the anti-CD4 scFv comprises a CDR-H1, a CDR-H2, and a CDR-H3 comprising the amino acid sequence set forth in SEQ ID NO: 332, 333, and 334, respectively. In some embodiments, the anti-CD4 scFv comprises a CDR-L1, a CDR-L2, and a CDR-L3 comprising the amino acid sequence set forth in SEQ ID NO: 335, 336, and 331, respectively. In some embodiments, the anti-CD4 scFv comprises a CDR-H1, a CDR-H2, and a CDR-H3 comprising the amino acid sequence set forth in SEQ ID NO: 332, 333, and 334, respectively; and a CDR-L1, a CDR-L2, and a CDR-L3 comprising the amino acid sequence set forth in SEQ ID NO: 335, 336, and 331, respectively. In some embodiments, the anti-CD4 scFv comprises a CDR-H1, a CDR-H2, and a CDR-H3 comprising the amino acid sequence set forth in SEQ ID NO: 337, 338, and 334, respectively. In some embodiments, the anti-CD4 scFv comprises a CDR-L1, a CDR-L2, and a CDR-L3 comprising the amino acid sequence set forth in SEQ ID NO: 335, 336, and 331, respectively. In some embodiments, the anti-CD4 scFv comprises a CDR-H1, a CDR-H2, and a CDR-H3 comprising the amino acid sequence set forth in SEQ ID NO: 337, 338, and 334, respectively; and a CDR-L1, a CDR-L2, and a CDR-L3 comprising the amino acid sequence set forth in SEQ ID NO: 335, 336, and 331, respectively. In some embodiments, the anti-CD4 scFv comprises a heavy chain variable region (VH) comprising the amino acid sequence set forth in SEQ ID NO:339. In some embodiments, the anti-CD4 scFv comprises a light chain variable region (VL) comprising the amino acid sequence set forth in SEQ ID NO:340. In some embodiments, the anti-CD4 scFv comprises a heavy chain variable region (VH) comprising the amino acid sequence set forth in SEQ ID NO:339; and a light chain variable region (VL) comprising the amino acid sequence set forth in SEQ ID NO:340. In some embodiments, the VH and VL are joined by a linker. In some embodiments, the linker comprises the amino acid sequence set forth in SEQ ID NO:275. In some embodiments, the anti-CD4 scFv comprises the amino acid sequence set forth in SEQ ID NO:341.

In some embodiments, the anti-CD4 scFv comprises a CDR-H1, a CDR-H2, and a CDR-H3 comprising the amino acid sequence set forth in SEQ ID NO: 342, 343, and 344, respectively. In some embodiments, the anti-CD4 scFv comprises a CDR-L1, a CDR-L2, and a CDR-L3 comprising the amino acid sequence set forth in SEQ ID NO: 345, 346, and 347, respectively. In some embodiments, the anti-CD4 scFv comprises a CDR-H1, a CDR-H2, and a CDR-H3 comprising the amino acid sequence set forth in SEQ ID NO: 342, 343, and 344, respectively; and a CDR-L1, a CDR-L2, and a CDR-L3 comprising the amino acid sequence set forth in SEQ ID NO: 345, 346, and 347, respectively. In some embodiments, the anti-CD4 scFv comprises a CDR-H1, a CDR-H2, and a CDR-H3 comprising the amino acid sequence set forth in SEQ ID NO: 348, 349, and 350, respectively. In some embodiments, the anti-CD4 scFv comprises a CDR-L1, a CDR-L2, and a CDR-L3 comprising the amino acid sequence set forth in SEQ ID NO: 351, 352, and 347, respectively. In some embodiments, the anti-CD4 scFv comprises a CDR-H1, a CDR-H2, and a CDR-H3 comprising the amino acid sequence set forth in SEQ ID NO: 242, 243, and 244, respectively; and a CDR-L1, a CDR-L2, and a CDR-L3 comprising the amino acid sequence set forth in SEQ ID NO: 245, 246, and 198, respectively. In some embodiments, the anti-CD4 scFv comprises a CDR-H1, a CDR-H2, and a CDR-H3 comprising the amino acid sequence set forth in SEQ ID NO: 353, 354, and 350, respectively. In some embodiments, the anti-CD4 scFv comprises a CDR-L1, a CDR-L2, and a CDR-L3 comprising the amino acid sequence set forth in SEQ ID NO: 351, 353, and 347, respectively. In some embodiments, the anti-CD4 scFv comprises a CDR-H1, a CDR-H2, and a CDR-H3 comprising the amino acid sequence set forth in SEQ ID NO: 353, 354, and 350, respectively; and a CDR-L1, a CDR-L2, and a CDR-L3 comprising the amino acid sequence set forth in SEQ ID NO: 351, 352, and 347, respectively. In some embodiments, the anti-CD4 scFv comprises a heavy chain variable region (VH) comprising the amino acid sequence set forth in SEQ ID NO:355. In some embodiments, the anti-CD4 scFv comprises a light chain variable region (VL) comprising the amino acid sequence set forth in SEQ ID NO:356. In some embodiments, the anti-CD4 scFv comprises a heavy chain variable region (VH) comprising the amino acid sequence set forth in SEQ ID NO:355; and a light chain variable region (VL) comprising the amino acid sequence set forth in SEQ ID NO:356. In some embodiments, the VH and VL are joined by a linker. In some embodiments, the linker comprises the amino acid sequence set forth in SEQ ID NO:275. In some embodiments, the anti-CD4 scFv comprises the amino acid sequence set forth in SEQ ID NO:357.

In some embodiments, the anti-CD4 antibody or antigen-binding fragment is a single domain antibody. In some embodiments, the anti-CD4 antibody or antigen-binding fragment is a camelid (e.g. llama, alpaca, camel) anti-CD4 antibody or antigen-binding fragment (e.g. VHH). In some embodiments, the anti-CD4 antibody or antigen-binding fragment is an anti-CD4 VHH. In some embodiments, the anti-CD4 VHH comprises a CDR-H1, a CDR-H2, and a CDR-H3 comprising the amino acid sequence set forth in SEQ ID NO: 358, 359, and 360, respectively. In some embodiments, the anti-CD4 VHH comprises a CDR-H1, a CDR-H2, and a CDR-H3 comprising the amino acid sequence set forth in SEQ ID NO: 361, 362, and 363, respectively. In some embodiments, the anti-CD4 VHH comprises a CDR-H1, a CDR-H2, and a CDR-H3 comprising the amino acid sequence set forth in SEQ ID NO: 364, 365, and 363, respectively. In some embodiments, the anti-CD4 VHH comprises the amino acid sequence set forth in SEQ ID NO:366.

### c) CD7 Binding Agents

In some embodiments, the lipid particles disclosed herein comprise one or more retargeted attachment proteins, each independently comprising (i) a paramyxovirus envelope attachment protein; and (ii) a targeting moiety directed to a target molecule expressed on the surface of a target cell, wherein the target molecule is CD7. In some embodiments, the targeting moiety is a CD7 binding domain, e.g., a CD7 binding agent, such as any of those disclosed herein.

The lipid particles disclosed herein include, in some embodiments, one or more CD7 binding agents. For example, a CD7 binding agent may be fused to or incorporated in a protein fusogen or attachment protein. In another embodiment, a CD7 binding agent may be incorporated into the lipid particle envelope via fusion with a transmembrane domain.

In some embodiments, CD7 binding domain may be fused to or incorporated in a protein fusogen or lipid particle envelope attachment protein (e.g., a retargeted attachment protein). In some embodiments, the CD7 binding domain may be incorporated into the co-display protein. In some embodiments, a CD7 binding domain may be incorporated into the viral envelope via fusion with a transmembrane domain. In some embodiments, the CD7 binding domain targets the lipid particle to a T cell.

Exemplary CD7 binding agents include antibodies and fragments thereof (e.g., scFv, VHH) that bind to CD7. Such antibodies may be derived from any species, and may be for example, mouse, rabbit, human, humanized, or camelid antibodies. Exemplary antibodies include grsinilimab, SPV-T3a and those disclosed in WO2015/184941; U.S. Ser. No. 10/106,609; WO2017/213979; WO2018/098306; WO2019086534; U.S. Ser. No. 11/447,548; WO2019/102234; WO2022/136887; WO2022/136888; WO2020/212710; WO2021/160267; WO2022/095803; WO2022/151851. Further exemplary anti-CD7 binding agents and G proteins are described in U.S. provisional application No. 63/172,518, which is incorporated by reference herein. Other exemplary binding agents include designed ankyrin repeat proteins (DARPins) and binding agents based on fibronectin type III (Fn3) scaffolds.

In some embodiments, protein fusogens or attachment proteins may be re-targeted by mutating amino acid residues in a fusion protein or a targeting protein (e.g. retargeted attachment protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type NiV-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi:10.1038/nbt942, Molecular Therapy vol. 16 no. 8, 1427-1436 August 2008, doi:10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens (e.g., attachment proteins) may be re-targeted by covalently conjugating a CD7 binding agent to the fusion protein or attachment protein (e.g. retargeted attachment protein). In some embodiments, the fusogen and CD7 binding agent are covalently conjugated by expression of a chimeric protein comprising the fusogen linked to the CD8 binding agent. In some embodiments, a single-chain variable fragment (scFv) can be conjugated to fusogens to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a CD7 binding agent to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nml192). In some embodiments, altered and non-altered fusogens may be displayed on the same retroviral vector or VLP (doi: 10.1016/j.biomaterials.2014.01.051).

In some embodiments, a CD7 binding agent comprises a humanized antibody molecule, intact IgA, IgG, IgE or IgM antibody; bi- or multi-specific antibody (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In some embodiments, the CD7 binding agent is a peptide. In some embodiments, the CD7 binding agent is an antibody, such as a single-chain variable fragment (scFv). In some embodiments, the CD7 binding agent is an antibody, such as a single domain antibody. In some embodiments, the CD7 binding agent is a VHH. In some embodiments, the antibody can be human or humanized. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD7 binding agent is attached to the C-terminus of the G protein (e.g., fusogen) or biologically active portion thereof. In some embodiments, the N-terminus of the CD7 binding agent is exposed on the exterior surface of the lipid bilayer.

In some embodiments, the CD7 binding agent is the only surface displayed non-viral sequence of the viral vector. In some embodiments, the CD7 binding agent is the only membrane bound non-viral sequence of the viral vector. In some embodiments, the viral vector does not contain a molecule that engages or stimulates T cells other than the CD7 binding agent.

In some embodiments, viral vectors may display CD7 binding agents that are not conjugated to protein fusogens in order to redirect the fusion activity towards a cell that is bound by the targeting moiety, or to affect homing.

### d) CD8 Binding Agents

In some embodiments, the lipid particles disclosed herein comprise one or more retargeted attachment proteins, each independently comprising (i) a paramyxovirus envelope attachment protein; and (ii) a targeting moiety directed to a target molecule expressed on the surface of a target cell, wherein the target molecule is CD8. In some embodiments, the targeting moiety is a CD8 binding domain, e.g., a CD8 binding agent, such as any of those disclosed herein.

The lipid particles disclosed herein include, in some embodiments, one or more CD8 binding agents. For example, a CD8 binding agent may be fused to or incorporated in a protein fusogen or attachment protein. In another embodiment, a CD8 binding agent may be incorporated into the lipid particle envelope via fusion with a transmembrane domain.

In some embodiments, CD8 binding domain may be fused to or incorporated in a protein fusogen or lipid particle envelope attachment protein (e.g., a retargeted attachment protein). In some embodiments, the CD8 binding domain may be incorporated into the co-display protein. In some embodiments, a CD8 binding domain may be incorporated into the viral envelope via fusion with a transmembrane domain. In some embodiments, the CD8 binding domain targets the lipid particle to a T cell.

Exemplary CD8 binding agents include antibodies and fragments thereof (e.g., scFv, VHH) that bind to one or more of CD8 alpha and CD8 beta. Such antibodies may be derived from any species, and may be for example, mouse, rabbit, human, humanized, or camelid antibodies. Exemplary antibodies include those disclosed in WO2014025828, WO2014164553, WO2020069433, WO2015184203, US20160176969, WO2017134306, WO2019032661, WO2020257412, WO2018170096, WO2020060924, U.S. Ser. No. 10/730,944, US20200172620, and the non-human antibodies OKT8; RPA-T8, 12.C7 (Novus); 17D8, 3B5, LT8, RIV11, SP16, YTC182.20, MEM-31, MEM-87, RAVB3, C8/144B (Thermo Fisher); 2ST8.5H7, Bu88, 3C39, Hit8a, SPM548, CA-8, SKI, RPA-T8 (GeneTex); UCHT4 (Absolute Antibody); BW135/80 (Miltenyi); G42-8 (BD Biosciences); C8/1779R, mAb 104 (Enzo Life Sciences); B-Z31 (Sapphire North America); 32-M4, 5F10, MCD8, UCH-T4, 5F2 (Santa Cruz); D8A8Y, RPA-T8 (Cell Signaling Technology). Further exemplary anti-CD8 binding agents and G proteins are described in U.S. provisional application No. 63/172,518, which is incorporated by reference herein. Other exemplary binding agents include designed ankyrin repeat proteins (DARPins) and binding agents based on fibronectin type III (Fn3) scaffolds.

In some embodiments, the CD8 binding agent comprises a CDR-H1, a CDR-H2, and a CDR-H3 comprising the amino acid sequence set forth in SEQ ID NO: 483, 484, and 485, respectively; and a CDR-L1, a CDR-L2, and a CDR-L3 comprising the amino acid sequence set forth in SEQ ID NO: 486, 487, and 488, respectively. In some embodiments, the CD8 binding agent comprises a heavy chain variable region (VH) comprising the amino acid sequence set forth in SEQ ID NO:369, and a light chain variable region (VL) comprising the amino acid sequence set forth in SEQ ID NO:370. In some embodiments, the CD8 binding agent comprises the sequence set forth in SEQ ID NO:489.

In some embodiments, the CD8 binding agent comprises a CDR-H1, a CDR-H2, and a CDR-H3 comprising the amino acid sequence set forth in SEQ ID NO: 490, 491, and 492, respectively; and a CDR-L1, a CDR-L2, and a CDR-L3 comprising the amino acid sequence set forth in SEQ ID NO: 493, 494, and 495, respectively. In some embodiments, the CD8 binding agent comprises a heavy chain variable region (VH) comprising the amino acid sequence set forth in SEQ ID NO:371 and a light chain variable region (VL) comprising the amino acid sequence set forth in SEQ ID NO:372. In some embodiments, the CD8 binding agent comprises the sequence set forth in SEQ ID NO:496.

In some embodiments, the CD8 binding agent comprises a CDR-H1, a CDR-H2, and a CDR-H3 comprising the amino acid sequence set forth in SEQ ID NO: 497, 498, and 499, respectively; and a CDR-L1, a CDR-L2, and a CDR-L3 comprising the amino acid sequence set forth in SEQ ID NO: 486, 487, and 500, respectively. In some embodiments, the CD8 binding agent comprises a heavy chain variable region (VH) comprising the amino acid sequence set forth in SEQ ID NO:373, and a light chain variable region (VL) comprising the amino acid sequence set forth in SEQ ID NO:374. In some embodiments, the CD8 binding agent comprises the sequence set forth in SEQ ID NO:501.

In some embodiments, the CD8 binding agent comprises a CDR-H1, a CDR-H2, and a CDR-H3 comprising the amino acid sequence set forth in SEQ ID NO: 502, 503, and 504, respectively; and a CDR-L1, a CDR-L2, and a CDR-L3 comprising the amino acid sequence set forth in SEQ ID NO: 505, 506, and 507, respectively. In some embodiments, the CD8 binding agent comprises a heavy chain variable region (VH) comprising the amino acid sequence set forth in SEQ ID NO:375, and a light chain variable region (VL) comprising the amino acid sequence set forth in SEQ ID NO:376. In some embodiments, the CD8 binding agent comprises the sequence set forth in SEQ ID NO:508.

In some embodiments, the CD8 binding agent comprises a CDR-H1, a CDR-H2, and a CDR-H3 comprising the amino acid sequence set forth in SEQ ID NO: 509, 510, and 511, respectively. In some embodiments, the CD8 binding agent comprises a heavy chain variable region (VH) comprising the amino acid sequence set forth in SEQ ID NO:377. In some embodiments, the CD8 binding agent comprises the sequence set forth in SEQ ID NO:377.

In some embodiments, the CD8 binding agent comprises any CD8 binding agent as described in US 2019/0144885, incorporated by reference herein in its entirety.

In some embodiments, the CD8 binding agent is an scFv that contains a VH and VL set forth from any as below, in which the VH and VL are separated by linker. In some embodiments, the CD8 binding agent is a VHH having the sequence set forth below. In some embodiments, the CD8 binding agent is linked to the C-terminus of a truncated NiV-G set forth in SEQ ID NO: 19 to provide a re-targeted NiV-G. In some embodiments, the retargeted NiV-G is pseudotyped on a lentiviral vector with the NiV-F (e.g. set forth in SEQ ID NO:227). In some embodiments, the lentiviral vector further contains a payload gene encoding an anti-CD19 CAR. In some embodiments, the anti-CD19 CAR contains an anti-CD19 FMC63 scFv binding domain set forth in SEQ ID NO:239, a CD8 hinge set forth in SEQ ID NO:367, a CD8 transmembrane domain set forth in SEQ ID NO: 368, a 4-1bb signaling domain set forth in SEQ ID NO:248. a CD3zeta signaling domain set forth in SEQ ID NO: 249.

CD8_1
VH (SEQ ID NO.: 369):
QVQLVQSGAEVKKPGASVKVSCKASGGTFSSYAISWVRQAPGQGL
EWMGIIDPSDGNTNYAQNFQGRVTMTRDTSTSTVYMELSSLRSED
TAVYYCAKERAAAGYYYYMDVWGQGTTVTVSS
VL (SEQ ID NO.: 370):
DIQMTQSPSSLSASVGDRVTITCRASQSISSYLNWYQQKPGKAPK
LLIYAASSLQSGVPSRFSGSGSGTDFTLTISSLQPEDFATYYCQQ
SYSTPLTFGGGTKVEIKR
CD8_2
VH (SEQ ID NO.: 371):
QVQLVQSGAEVKKPGASVKVSCKASGYTFTDYYIQWVRQAPGQGL
EWMGWINPNSGGTSYAQKFQGRVTMTRDTSTSTVYMELSSLRSED
TAVYYCAKEGDYYYGMDAWGQGTMVTVSS
VL (SEQ ID NO.: 372):
DIVMTQSPLSLPVTPGEPASISCRSSQSLLHSNGYNYLDWYLQKP
GQSPQLLIYLGSNRASGVPDRFSGSGSGTDFTLKISRVEAEDVGV
YYCMQGLQTPHTFGQGTKVEIKR
CD8_3
VH (SEQ ID NO.: 373):
QVQLVQSGAEVKKPGASVKVSCKASGYTFTSYYMHWVRQAPGQGL
EWMGGFDPEDGETIYAQKFQGRVTMTRDTSTSTVYMELSSLRSED
TAVYYCARDQGWGMDVWGQGTTVTVSS
VL(SEQ ID NO.: 374):
DIQMTQSPSSLSASVGDRVTITCRASQSISSYLNWYQQKPGKAPK
LLIYAASSLQSGVPSRFSGSGSGTDFTLTISSLQPEDFATYYCQQ
TYSTPYTFGQGTKLEIKR
CD8_4
VH (SEQ ID NO.: 375):
QVQLVQSGAEVKKPGASVKVSCKASGYTFTNHYMHWVRQAPGQGL
EWMGWMNPNSGNTGYAQKFQGRVTMTRDTSTSTVYMELSSLRSED
TAVYYCASSESGSDLDYWGQGTLVTVSS
VL (SEQ ID NO.: 376):
DIQMTQSPSSLSASVGDRVTITCRASQTIGNYVNWYQQKPGKAPK
LLIYGASNLHTGVPSRFSGSGSGTDFTLTISSLQPEDFATYYCQQ
TYSAPLTFGGGTKVEIKR

In some embodiments, the CD8 binding agent is VHH set forth as:

VHH (SEQ ID NO.: 377):
QVQLVESGGGLVQAGGSLRLSCAASGRTFSGYVMGWFRQAPGKQR
KFVAAISRGGLSTSYADSVKGRFTISRDNAKNTVFLQMNTLKPED
TAVYYCAADRSDLYEITAASNIDSWGQGTLVTVSS

In some embodiments, protein fusogens or attachment proteins may be re-targeted by mutating amino acid residues in a fusion protein or a targeting protein (e.g. retargeted attachment protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type NiV-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi:10.1038/nbt942, Molecular Therapy vol. 16 no. 8, 1427-1436 August 2008, doi:10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens (e.g., attachment proteins) may be re-targeted by covalently conjugating a CD8 binding agent to the fusion protein or attachment protein (e.g. retargeted attachment protein). In some embodiments, the fusogen and CD8 binding agent are covalently conjugated by expression of a chimeric protein comprising the fusogen linked to the CD8 binding agent. In some embodiments, a single-chain variable fragment (scFv) can be conjugated to fusogens to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a CD8 binding agent to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nml192). In some embodiments, altered and non-altered fusogens may be displayed on the same retroviral vector or VLP (doi: 10.1016/j.biomaterials.2014.01.051).

In some embodiments, a CD8 binding agent comprises a humanized antibody molecule, intact IgA, IgG, IgE or IgM antibody; bi- or multi-specific antibody (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In some embodiments, the CD8 binding agent is a peptide. In some embodiments, the CD8 binding agent is an antibody, such as a single-chain variable fragment (scFv). In some embodiments, the CD8 binding agent is an antibody, such as a single domain antibody. In some embodiments, the CD8 binding agent is a VHH. In some embodiments, the antibody can be human or humanized. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD8 binding agent is attached to the C-terminus of the G protein (e.g., fusogen) or biologically active portion thereof. In some embodiments, the N-terminus of the CD8 binding agent is exposed on the exterior surface of the lipid bilayer.

In some embodiments, the CD8 binding agent is the only surface displayed non-viral sequence of the viral vector. In some embodiments, the CD8 binding agent is the only membrane bound non-viral sequence of the viral vector. In some embodiments, the viral vector does not contain a molecule that engages or stimulates T cells other than the CD8 binding agent.

In some embodiments, viral vectors may display CD8 binding agents that are not conjugated to protein fusogens in order to redirect the fusion activity towards a cell that is bound by the targeting moiety, or to affect homing.

### II. HSC Binding Domains

In some embodiments, the lipid particles disclosed herein comprise one or more retargeted attachment proteins, each independently comprising (i) a paramyxovirus envelope attachment protein; and (ii) a targeting moiety directed to a target molecule expressed on the surface of a target cell. In some embodiments, the targeting moiety is an HSC binding domain, e.g., an HSC binding agent, such as any of those disclosed herein.

In some embodiments, an HSC binding domain may be fused to or incorporated in a protein fusogen or lipid particle envelope attachment protein (e.g., a retargeted attachment protein). In some embodiments, the HSC binding domain may be incorporated into the co-display protein. In some embodiments, an HSC binding domain may be incorporated into the viral envelope via fusion with a transmembrane domain. In some embodiments, the HSC binding domain targets the lipid particle to a T cell.

The lipid particles disclosed herein include, in some embodiments, one or more HSC binding domains (e.g., HSC binding agent) that target the viral vector to a cell that is an HSC. In some embodiments, the HSC binding agent binds to a molecule expressed on the surface of the HSC. The cell surface molecule may be a receptor, coreceptor, or a GPI-anchored protein. In some embodiments, the HSC binding agent binds ASCT2, CD105, CD110, CD117, CD133, CD146, CD164, CD34, CD46, CD49f, CD90, EPCR, or ITGA3. In some embodiments, a HSC binding agent may be fused to or incorporated in a protein fusogen or lipid particle envelope attachment protein (e.g., a retargeted attachment protein). In some embodiments, a HSC binding agent may be incorporated into the viral envelope via fusion with a transmembrane domain. In some embodiments, the HSC binding agent targets the lipid particle to a HSC.

In particular embodiments, a HSC binding agent may be fused to or incorporated in a protein fusogen or attachment protein, thereby retargeting the lipid particle to a HSC. In some embodiments, for re-targeting the HSC binding agent is fused to a protein fusogen or envelope attachment protein that is mutated to reduce binding for the native binding partner of the fusogen or viral envelope protein. In some embodiments, the fusogen is or contains a mutant G protein or a biologically active portion thereof that is a mutant of wild-type NiV-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi:10.1038/nbt942, Molecular Therapy vol. 16 no. 8, 1427-1436 August 2008, doi:10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens may be re-targeted by covalently conjugating a HSC binding agent to the attachment protein. In some embodiments, the fusogen and HSC binding agent are covalently conjugated by expression of a chimeric protein comprising the fusogen linked to the HSC binding agent (e.g., retargeted attachment protein). The HSC binding agent can include any targeting protein able to confer specific binding to a target molecule expressed on the surface of a HSC. In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, the HSC binding agent is an antibody or antigen binding fragment thereof. In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nml192). In some embodiments, altered and non-altered fusogens may be displayed on the same retroviral vector or VLP (doi: 10.1016/j.biomaterials.2014.01.051).

In some embodiments, a single-chain variable fragment (scFv) can be conjugated to fusogens to redirect fusion activity towards HSCs that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards HSCs that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, a single domain antibody (e.g., a VHH) can be conjugated to fusogens to redirect fusion activity towards HSCs that display the sdAb binding target. In some embodiments, receptor ligands and antigens can be conjugated to fusogens to redirect fusion activity towards HSCs that display the target receptor (DOI: 10.1089/hgtb.2012.054, DOI: 10.1128/JVI.76.7.3558-3563.2002).

In some embodiments, the target cell is a CD34+ progenitor cells. In some embodiments, the target cell molecule is expressed on at least a subset of CD34+ progenitor cells.

In some embodiments, the cell surface molecule is expressed on HSCs. In some embodiments, the cell surface molecule is expressed on MPPs. In some embodiments, the cell surface molecule is expressed on MLPs. In some embodiments, the cell surface molecule is expressed on ETPs. In some embodiments, the cell surface molecule is expressed on MEPs. In some embodiments, the cell surface molecule is expressed on CMPs. In some embodiments, the cell surface molecule is expressed on GMPs. In some embodiments, the cell surface molecule is expressed on any combination of the foregoing CD34+ progenitor subpopulations. In some embodiments, the cell surface molecule is expressed on HSCs and MPPs. In some embodiments, the cell surface molecule is expressed on myeloid progenitors. In some embodiments, the cell surface molecule is expressed on lymphoid progenitors. In some embodiments, the cell surface molecule is expressed on myeloid progenitors. In some embodiments, the cell surface molecule is expressed on HSCs, MPPs, MEPs, CMPs, and GMPs.

In some embodiments, the cell surface molecule is ASCT2. In some embodiments, the target cell is ASCT2+.

In some embodiments, the cell surface molecule is CD105. In some embodiments, the target cell is CD105+.

In some embodiments, the cell surface molecule is CD110. In some embodiments, the target cell is CD110+.

In some embodiments, the cell surface molecule is CD117. In some embodiments, the target cell is CD117+.

In some embodiments, the cell surface molecule is CD133. In some embodiments, the target cell is CD133+.

In some embodiments, the cell surface molecule is CD146. In some embodiments, the target cell is CD146+.

In some embodiments, the cell surface molecule is CD164. In some embodiments, the target cell is CD164+.

In some embodiments, the cell surface molecule is CD34. In some embodiments, the target cell is CD34+.

In some embodiments, the cell surface molecule is CD46. In some embodiments, the target cell is CD46+.

In some embodiments, the cell surface molecule is CD49f. In some embodiments, the target cell is CD49f+.

In some embodiments, the ta cell surface molecule is CD90. In some embodiments, the target cell is CD90+.

In some embodiments, the cell surface molecule is EPCR. In some embodiments, the target cell is EPCR+.

In some embodiments, the cell surface molecule is ITGA3. In some embodiments, the target cell is ITGA3+.

In some embodiments, the target molecule is CD133. In some embodiments, the target cell is CD133+. In some embodiments, the targeting agent is an anti-CD133 antibody. Exemplary anti-CD133 antibodies include CART133, AC133, 293C3-SDIE, CMab-43, RWO3, 293C3H9 (293C3), and W6B3H10 (W6B3); and anti-CD133 antibodies disclosed in U.S. Pat. Nos. 8,722,858, 9,249,225, 9,624,303, U.S. Ser. No. 10/106,623, U.S. Ser. No. 10/711,068, U.S. Ser. No. 11/098,109, U.S. Ser. No. 11/214,628, U.S. Ser. No. 11/352,435, and U.S. Ser. No. 11/220,551; US Patent Application Nos. US20130224202; PCT Application Nos. WO200901840, WO2011089211, WO2011149493, WO2014128185, WO2015121383, WO2016154623, WO2018045880, WO2018072025, and WO2022022718; and Canadian Patent Application No. CA2962157.

In some embodiments, the lipid particles disclosed herein comprise one or more retargeted attachment proteins, each independently comprising (i) a paramyxovirus envelope attachment protein; and (ii) a targeting moiety directed to a target molecule expressed on the surface of a target cell, wherein the target molecule is CD133. In some embodiments, the targeting moiety is a CD133 binding domain, e.g., a CD133 binding agent, such as any of those disclosed herein.

In some embodiments, the lipid particles comprise one or more HSC binding domains that is a CD133 binding agent that targets the viral vector to a cell that is an HSC. In some embodiments, the lipid particles comprise two or more HSC binding domains that are each a CD133 binding agent that targets the viral vector to a cell that is an HSC. In some embodiments, each of the two or more HSC binding domains that are each a CD133 binding agent bind distinct epitopes of the same target molecule (CD133). In some embodiments, the lipid particle comprises two or more, e.g., two, three, four, or five or more, CD133 binding agents.

In some embodiments, the lipid particle comprises one or more targeting moieties, e.g., HSC binding domains, in which the CD133 binding agent is an antibody or antigen-binding fragment. In some embodiments, the CD133 binding agent is a single chain antibody, such as a single chain variable fragment (scFv). In some embodiments, the CD133 binding agent contains a variable heavy (VH) chain comprising a CDR-H1, a CDR-H2, and a CDR-H3 comprising the amino acid sequences of SEQ ID NOs: 536, 537, and 538, respectively, and a variable light (VL) chain comprising a CDR-L1, a CDR-L2, and a CDR-L3 comprising the amino acid sequences of SEQ ID NOs: 540, 541, and 542, respectively; (b) a VH chain comprising a CDR-H1, a CDR-H2, and a CDR-H3 comprising the amino acid sequences of SEQ ID NOs: 545, 546, and 547, respectively, and a VL chain comprising a CDR-L1, a CDR-L2, and a CDR-L3 comprising the amino acid sequences of SEQ ID NOs: 549, 550, and 551, respectively; (c) a VH chain comprising a CDR-H1, a CDR-H2, and a CDR-H3 comprising the amino acid sequences of SEQ ID NOs: 518, 519, and 520, respectively, and a VL chain comprising a CDR-L1, a CDR-L2, and a CDR-L3 comprising the amino acid sequences of SEQ ID NOs: 522, 523, and 524, respectively; (d) a VH chain comprising a CDR-H1, a CDR-H2, and a CDR-H3 comprising the amino acid sequences of SEQ ID NOs: 527, 528, and 529, respectively, and a VL chain comprising a CDR-L1, a CDR-L2, and a CDR-L3 comprising the amino acid sequences of SEQ ID NOs: 531, 532, and 533, respectively; or (e) a VH chain comprising a CDR-H1, a CDR-H2, and a CDR-H3 comprising the amino acid sequences of SEQ ID NOs: 554, 555, and 556, respectively, and a VL chain comprising a CDR-L1, a CDR-L2, and a CDR-L3 comprising the amino acid sequences of SEQ ID NOs: 558, 559, and 560, respectively.

In some embodiments, the lipid particle comprises one or more targeting moieties, e.g., HSC binding domains, that is a CD133 binding agent which is a single chain antibody, such as a single chain variable fragment (scFv) comprising a VH and a VL selected from: (a) a VH comprising a CDR-H1, a CDR-H2, and a CDR-H3 comprising the amino acid sequences of SEQ ID NOs: 289, 565, and 538, respectively, and a VL comprising a CDR-L1, a CDR-L2, and a CDR-L3 comprising the amino acid sequences of SEQ ID NOs: 540, 541, and 542, respectively; (b) a VH comprising a CDR-H1, a CDR-H2, and a CDR-H3 comprising the amino acid sequences of SEQ ID NOs: 566, 567, and 547, respectively, and a VL comprising a CDR-L1, a CDR-L2, and a CDR-L3 comprising the amino acid sequences of SEQ ID NOs: 549, 550, and 551, respectively; (c) a VH comprising a CDR-H1, a CDR-H2, and a CDR-H3 comprising the amino acid sequences of SEQ ID Nos: 568, 569, and 520, respectively, and a VL comprising a CDR-L1, a CDR-L2, and a CDR-L3 comprising the amino acid sequences of SEQ ID NOs: 522, 523, and 524, respectively; (d) a VH comprising a CDR-H1, a CDR-H2, and a CDR-H3 comprising the amino acid sequences of SEQ ID NOs: 570, 571, and 529, respectively, and a VL comprising a CDR-L1, a CDR-L2, and a CDR-L3 comprising the amino acid sequences of SEQ ID NOs: 531, 532, and 533, respectively; and (e) a VH comprising a CDR-H1, a CDR-H2, and a CDR-H3 comprising the amino acid sequences of SEQ ID NOs: 572, 573, and 556, respectively, and a VL comprising a CDR-L1, a CDR-L2, and a CDR-L3 comprising the amino acid sequences of SEQ ID Nos: 558, 559, and 560, respectively.

In some embodiments, the lipid particle comprises one or more, e.g., one, two, three, or more, targeting moieties, e.g., HSC binding domains, independently selected from: (a) a CD133 binding agent comprising a heavy chain variable (VH) region comprising the amino acid sequence of SEQ ID NO: 535, or an amino acid sequence having at least 90%, 91%, 92%, 93%, 94%, 95%, 96%, 97%, 98%, or 99% sequence identity thereto; and a light chain variable (VL) region comprising the amino acid sequence of SEQ ID NO: 539, or an amino acid sequence having at least 90%, 91%, 92%, 93%, 94%, 95%, 96%, 97%, 98%, or 99% sequence identity thereto; (b) a CD133 binding agent comprising a VH region comprising the amino acid sequence of SEQ ID NO: 544, or an amino acid sequence having at least 90%, 91%, 92%, 93%, 94%, 95%, 96%, 97%, 98%, or 99% sequence identity thereto; and a VL region comprising the amino acid sequence of SEQ ID NO: 548, or an amino acid sequence having at least 90%, 91%, 92%, 93%, 94%, 95%, 96%, 97%, 98%, or 99% sequence identity thereto; (c) a CD133 binding agent comprising a VH region comprising the amino acid sequence of SEQ ID NO: 517, or an amino acid sequence having at least 90%, 91%, 92%, 93%, 94%, 95%, 96%, 97%, 98%, or 99% sequence identity thereto; and a VL region comprising the amino acid sequence of SEQ ID NO: 521, or an amino acid sequence having at least 90%, 91%, 92%, 93%, 94%, 95%, 96%, 97%, 98%, or 99% sequence identity thereto; (d) a CD133 binding agent comprising a VH region comprising the amino acid sequence of SEQ ID NO: 526, or an amino acid sequence having at least 90%, 91%, 92%, 93%, 94%, 95%, 96%, 97%, 98%, or 99% sequence identity thereto; and a VL region comprising the amino acid sequence of SEQ ID NO: 530, or an amino acid sequence having at least 90%, 91%, 92%, 93%, 94%, 95%, 96%, 97%, 98%, or 99% sequence identity thereto; and (e) a CD133 binding agent comprising a VH region comprising the amino acid sequence of SEQ ID NO: 553, or an amino acid sequence having at least 90%, 91%, 92%, 93%, 94%, 95%, 96%, 97%, 98%, or 99% sequence identity thereto; and a VL region comprising the amino acid sequence of SEQ ID NO: 557, or an amino acid sequence having at least 90%, 91%, 92%, 93%, 94%, 95%, 96%, 97%, 98%, or 99% sequence identity thereto.

In some embodiments, the lipid particle comprises one or more, e.g., one, two, three, or more, targeting moieties, e.g., HSC binding domains, independently selected from: (a) a CD133 binding agent comprising a heavy chain variable (VH) region comprising the amino acid sequence of SEQ ID NO: 535 and a light chain variable (VL) region comprising the amino acid sequence of SEQ ID NO: 539; (b) a CD133 binding agent comprising a VH region comprising the amino acid sequence of SEQ ID NO: 544 and a VL region comprising the amino acid sequence of SEQ ID NO: 548; (c) a CD133 binding agent comprising a VH region comprising the amino acid sequence of SEQ ID NO: 517 and a VL region comprising the amino acid sequence of SEQ ID NO: 521; (d) a CD133 binding agent comprising a VH region comprising the amino acid sequence of SEQ ID NO: 526 and a VL region comprising the amino acid sequence of SEQ ID NO: 530; and (e) a CD133 binding agent comprising a VH region comprising the amino acid sequence of SEQ ID NO: 553 and a VL region comprising the amino acid sequence of SEQ ID NO: 557.

In some embodiments, the lipid particle comprises one or more targeting moieties, e.g., HSC binding domains, that is an scFv comprising an amino acid sequence independently selected from the group consisting of SEQ ID NOs: 516, 525, 534, 543, and 552, or an amino acid sequence having at least 90%, 91%, 92%, 93%, 94%, 95%, 96%, 97%, 98%, or 99% sequence identity thereto. In some embodiments, each of the one or more CD133 binding agents is independently an scFv comprising an amino acid sequence selected from the group consisting of SEQ ID NOs: 516, 525, 534, 543, and 552, or an amino acid sequence having at least 90%, 91%, 92%, 93%, 94%, 95%, 96%, 97%, 98%, or 99% sequence identity thereto.

In some embodiments, the lipid particle comprises a first CD133 binding agent that is an scFv comprising the amino acid sequence of SEQ ID NO: 516 or an amino acid sequence having at least 90%, 91%, 92%, 93%, 94%, 95%, 96%, 97%, 98%, or 99% sequence identity thereto, and a second CD133 binding agent that is an scFv comprising the amino acid sequence of SEQ ID NO: 525 or an amino acid sequence having at least 90%, 91%, 92%, 93%, 94%, 95%, 96%, 97%, 98%, or 99% sequence identity thereto.

In some embodiments, the target molecule is CD105. In some embodiments, the target cell is CD105+. In some embodiments, the targeting agent is an anti-CD105 antibody. Exemplary anti-CD105 antibodies include carotuximab, TRC105, huRHI05, and TCR205; and anti-CD105 antibodies disclosed in U.S. Pat. Nos. 8,221,753, 8,609,094, 9,150,652, 95,181,212, 9,926,375, 9,944,714, U.S. Ser. No. 10/155,820, and U.S. Ser. No. 10/336,831; US Patent Application Nos. US20100098692, US20100196398, US20170007714, and US20220233591; PCT Application Nos. WO2010039873, WO2011041441, WO2016077451, WO2018067819, WO2010032059, WO2012149412, WO2015118031, WO2021118955, and WO2021118957; and Korean Patent No. KR101398707B1.

In some embodiments, the lipid particles disclosed herein comprise one or more retargeted attachment proteins, each independently comprising (i) a paramyxovirus envelope attachment protein; and (ii) a targeting moiety directed to a target molecule expressed on the surface of a target cell, wherein the target molecule is CD117. In some embodiments, the targeting moiety is a CD117 binding domain, e.g., a CD117 binding agent, such as any of those disclosed herein.

In some embodiments, the target molecule is CD117. In some embodiments, the target cell is CD117+. In some embodiments, the targeting moiety is a CD117 binding agent that is an anti-CD117 antibody. In some embodiments, the CD117 binding agent is a single domain antibody, such as a VHH. In some embodiments, the lipid particles comprise one or more HSC binding domains that is a CD117 binding agent that targets the viral vector to a cell that is an HSC. In some embodiments, the lipid particle comprises one or more targeting moieties, e.g., HSC binding domains, that is a CD117 binding agent comprising an amino acid sequence independently selected from the group consisting of SEQ ID NOs: 512-515 and 575-621, or an amino acid sequence having at least 90%, 91%, 92%, 93%, 94%, 95%, 96%, 97%, 98%, or 99% sequence identity thereto.

In some embodiments, the lipid particle comprises one or more targeting moieties, e.g., HSC binding domains, selected from: (a) a CD117 binding agent comprising a VHH comprising the amino acid sequence of SEQ ID NO: 512, or an amino acid sequence having at least 90%, 91%, 92%, 93%, 94%, 95%, 96%, 97%, 98%, or 99% sequence identity thereto; (b) a CD117 binding agent comprising a VHH comprising the amino acid sequence of SEQ ID NO: 513, or an amino acid sequence having at least 90%, 91%, 92%, 93%, 94%, 95%, 96%, 97%, 98%, or 99% sequence identity thereto; (c) a CD117 binding agent comprising a VHH comprising the amino acid sequence of SEQ ID NO: 514, or an amino acid sequence having at least 90%, 91%, 92%, 93%, 94%, 95%, 96%, 97%, 98%, or 99% sequence identity thereto; and (d) a CD117 binding agent comprising a VHH comprising the amino acid sequence of SEQ ID NO: 515, or an amino acid sequence having at least 90%, 91%, 92%, 93%, 94%, 95%, 96%, 97%, 98%, or 99% sequence identity thereto.

In some embodiments, the lipid particle comprises one or more targeting moieties, e.g., HSC binding domains, each independently comprising a VHH comprising an amino acid sequence selected from the group consisting of SEQ ID NOs: 512-515 and 575-621, or an amino acid sequence having at least 90%, 91%, 92%, 93%, 94%, 95%, 96%, 97%, 98%, or 99% sequence identity to an amino acid sequence selected from the group consisting of SEQ ID NOs: 512-515 and 575-621, wherein the target molecule is CD117.

In some embodiments, the lipid particle comprises one or more targeting moieties, e.g., HSC binding domains, each independently comprising a VHH comprising a CDR-H1, a CDR-H2, and a CDR-H3 contained within an amino acid sequence selected from the group consisting of SEQ ID NOs: 512-515 and 575-621, wherein the target molecule is CD117.

In some embodiments, the target molecule is EPCR. In some embodiments, the target cell is EPCR+. In some embodiments, the targeting agent is an anti-EPCR antibody. Exemplary anti-EPCR antibodies include JRK1494, JRK1535; and anti-EPCR antibodies disclosed in US Patent Application Nos. US20210355231 and US20220127374; and PCT Application Nos. WO2020051277 and WO2020161478.

In some embodiments, the target molecule is CD34. In some embodiments, the target cell is CD34+. In some embodiments, the targeting agent is an anti-CD34 antibody. Exemplary anti-CD34 antibodies include h4C8, 9C5; and anti-CD34 antibodies disclosed in U.S. Pat. Nos. 8,399,249, 8,927,696, and U.S. Ser. No. 10/106,623; US Patent Application Nos. US20090221003, US20130143238, US20100311955, US20130172533, US20170320966, US20170298148, US20180169177, US20190135945; and PCT Application Nos. WO2009079922 and WO2015121383.

In some embodiments, the target molecule is ASCT2. In some embodiments, the target cell is ASCT2+. In some embodiments, the targeting agent is an anti-ASCT2 antibody. Exemplary anti-ASCT2 antibodies include idactamab, MEDI7247, KM4008, KM4012, KM4018; and anti-ASCT2 antibodies disclosed in U.S. Pat. Nos. 8,268,592, 8,501,180, 8,945,870, 8,673,592, and U.S. Ser. No. 10/829,554; US Patent Application Nos. US20180273617, US20190367605, US20210024629; and PCT Application Nos. WO2017083451, WO2018089393.

In some embodiments, the target molecule is CD90. In some embodiments, the target cell is CD90+. In some embodiments, the targeting agent is an anti-CD90 antibody. Exemplary anti-CD90 antibodies include EPR3133, CL1028, CL1040, AF-9, JF10-09, 5E10, 7E1B11; and anti-CD90 antibodies disclosed in US Patent Application No. US20210054068; and PCT Application No. WO2017214050.

In some embodiments, the target molecule is CD164. In some embodiments, the target cell is CD164+. In some embodiments, the targeting agent is an anti-CD164 antibody. Exemplary anti-CD164 antibodies include 67D2, H-4, 32G1, EML2058, 5C5, N6B6, 4B4, and 15-11-14; and anti-CD164 antibodies disclosed in PCT Application No. WO2006002438; and German Patent Nos. DE19727813C1 and DE19727815C1.

In some embodiments, the target molecule is CD49f. In some embodiments, the target cell is CD49f+. In some embodiments, the targeting agent is an anti-CD49f antibody. Exemplary anti-CD49f antibodies include CL6957, GoH3, SR45-00, and MP4F10; and anti-CD49f antibodies disclosed in U.S. Pat. No. 5,538,725, U.S. Ser. No. 10/030,071; US Patent Application Nos. US20110301227, US20160194400, US20160280789; and PCT Application Nos. WO2015034052 and WO2018127655.

In some embodiments, the target molecule is CD146. In some embodiments, the target cell is CD146+. In some embodiments, the targeting agent is an anti-CD146 antibody. Exemplary anti-CD146 antibodies include imaprelimab, PRX003, ABX-MA1, huAA98, M2H-1, M2J-1, and JM1-24-3; and anti-CD146 antibodies disclosed in U.S. Pat. Nos. 6,924,360, 7,067,131, US709844, U.S. Pat. Nos. 9,447,190, 9,782,500, U.S. Ser. No. 10/407,506, U.S. Ser. No. 10/414,825, U.S. Ser. No. 10/407,507, U.S. Ser. No. 10/584,177, U.S. Ser. No. 10/905,771, U.S. Ser. No. 11/427,648; US Patent Application Nos. US20030147809, US20040115205, US20060246077, US20140314744, US20150239980, US20140227292, US20160206764, US20190192573, US20170002089, US20150259419, US20170037144, US20170129954, US20170101470, US20180271994, US20180105602, US20200010563, US20200165336, US20200216560, US20200262929, US20200100838, US20220041748, US20220041749; and PCT Application Nos. WO2003057006, WO2003057837, WO2003057838, WO2012170071, WO2014000338, WO2015044218, WO2015136469, WO2015136470, WO2017046776, WO2017046774, WO2017149513, WO2017153953, WO2017208210, WO2018033630, WO2018220467, WO2018223140, WO2019068842, WO2019133639, WO2019137309, WO2020132190, WO2020132232, and WO2022082073.

Further exemplary targeting agents and corresponding target molecules are described in the table below. The targeting agent can be any described in the referenced associated documents that bind to the associated target molecule.

Table 3: Exemplary targeting agents and corresponding target molecules

TABLE 3
Exemplary targeting agents and corresponding target molecules

| | | Associated | |
|---|---|---|---|
| Target Molecule | Targeting Agent | Documents | Company/Institution |
| CD117 (c-Kit) | N/A | WO2022187050 | Biolegend |
| CD117 (c-Kit) | Briquilimab (also | US20130288303 | Amgen/Jasper |
| | known as JSP191 | US8791249 | Therapeutics |
| | or AMG191) | US20110223165 | |
| | | US8436150 | |
| | | WO2007127317 | |
| | | US7915391 | |
| CD117 (c-Kit) | CDX-0158 (also | US20200385483 | Celldex |
| | known as | US20220010027 | |
| | KTN0158) | US10774146 | |
| | | US20190153094 | |
| | | US20190106510 | |
| | | US10781267 | |
| | | US20190100598 | |
| | | US10793639 | |
| | | US20170158778 | |
| | | US10184007 | |
| | | US20170073422 | |
| | | US10189907 | |
| | | US20160311924 | |
| | | US9605081 | |
| | | WO2015179737 | |
| | | US20170121408 | |
| | | US10239943 | |
| | | WO2015112822 | |
| | | WO2015050959 | |
| | | WO2014018625 | |
| | | US20140065168 | |
| | | US9334332 | |
| | | WO2012103165 | |
| | | US20140056905 | |
| | | US9540443 | |
| | | WO2011119948 | |
| | | US20130011406 | |
| CD117 (c-Kit) | CDX-0159 | WO2022159737 | Celldex |
| CD117 (c-Kit) | | US20220127358 | Forty Seven |
| | | US20210122822 | |
| | | US11041022 | |
| | | WO2020242895 | |
| | | US20200165337 | |
| | | WO2020112687 | |
| | | US11208482 | |
| CD117 (c-Kit) | | WO2021099418 | IDD-Tech |
| CD117 (c-Kit) | | WO2012154861 | ImClone |
| | | US8552157 | |
| CD117 (c-Kit) | | WO2015067667 | INSERM |
| | | US20160264680 | |
| | | US9932410 | |
| CD117 (c-Kit) | LOP628 (also | US20220305134 | Novartis |
| | known as | US20200405875 | |
| | LMJ729) | US20200129632 | |
| | | WO2019244082 | |
| | | US20210228731 | |
| | | WO2018116178 | |
| | | US20190321483 | |
| | | US11357864 | |
| | | US20180125996 | |
| | | US20170035905 | |
| | | US10117953 | |
| | | WO2016020791 | |
| | | US20180193475 | |
| | | US10786578 | |
| | | US20150320880 | |
| | | US20170021033 | |
| | | US20140271688 | |
| | | WO2014150937 | |
| | | US20160030594 | |
| | | US9498543 | |
| | | US9789203 | |
| CD117 (c-Kit) | MGTA-117 (also | US20220177575 | Magenta Thera |
| | known as | US20220177578 | |
| | CD117-ADC or | US20220177577 | |
| | MGTA-C100) | US20220175944 | |
| | | US20220226493 | |
| | | US20210283267 | |
| | | US20210317228 | |
| | | US20200255523 | |
| | | WO2020219964 | |
| | | US20200376135 | |
| | | WO2020219748 | |
| | | WO2020219770 | |
| | | WO2020219774 | |
| | | WO2020219778 | |
| | | WO2020219775 | |
| | | WO2019234694 | |
| | | WO2019084067 | |
| | | WO2019084064 | |
| | | WO2019084057 | |
| | | WO2019084053 | |
| | | US20190153114 | |
| | | US10882915 | |
| | | US10899843 | |
| | | US20190134217 | |
| CD117 (c-Kit) | NN2101 (also | WO2020076105 | Ajou University |
| | known as 2G4) | WO2021107566 | |
| | | US20210355212 | |
| CD117 (c-Kit) | N/A (internal | WO2016033201 | Stanford University |
| | name SR1) | US20170224737 | |
| | | US10406179 | |
| CD117 (c-Kit) | N/A | US20200283521 | Granular |
| | | US11377493 | Therapeutics/ |
| | | US20200010548 | Ultrahuman Four |
| | | US10611838 | |
| | | WO2019155067 | |
| CD117 (c-Kit) | N/A | WO2021188590 | Magenta Thera |
| (CD117/CD3 | | | |
| bispecific) | | | |
| CD117 (c-Kit) | N/A | WO2021044008 | University of Zurich |
| (CD117/CD3 | | | |
| bispecific) | | | |
| CD110 (TPO | N/A | WO2011060076 | Amgen |
| Receptor/c-Mpl) | | US20120269814 | |
| CD110 (TPO | N/A (also known | US20120015436 | Chugai Pharmaceutical |
| Receptor/c-Mpl) | as TA136) | US8586039 | |
| | | US20090297501 | |
| | | US9493569 | |
| | | WO2006106903 | |
| | | US7691588 | |
| | | US8034903 | |
| CD110 (TPO | N/A | WO2022114603 | Daegu Gyeongbuk |
| Receptor/c-Mpl) | | | Institute of Science |
| | | | and Technology |
| CD110 (TPO | N/A | US20130053547 | Kyowa Hakko Kirin Co. |
| Receptor/c-Mpl) | | US8999329 | |
| | | US20120053326 | |
| | | US8236314 | |
| | | WO2009041734 | |
| | | US20100004429 | |
| | | US8048421 | |
| ITGA3 (Integrin | N/A | US20190309072 | Cytomx Therapeutics |
| alpha 3, VLA3A, | | WO2016179335 | |
| CD49c) | | US20160355592 | |
| | | US10233244 | |
| ITGA3 (Integrin | N/A (also | Ke et al. Cancer Sci. | Academia Sinica |
| alpha 3, VLA3A, | known as | 2020; 111: 3478- | |
| CD49c) | OV-Ab 30-7) | 3492. | |
| CD46 (MCP, | N/A | WO2021257542 | BioAtla |
| Membrane | | | |
| cofactor protein) | | | |
| CD46 (MCP, | FOR46 (also | WO2022150512 | Fortis Therapeutics |
| Membrane | known as | WO2022150514 | Inc/University of |
| cofactor protein) | YS5FL) | WO2022150517 | California |
| | | WO2022032020 | |
| | | US20200199245 | |
| | | US20200040094 | |
| | | US11208493 | |
| | | WO2018089807 | |
| | | US20190276553 | |
| | | US11434301 | |
| | | US20170240643 | |
| | | US10400045 | |
| | | WO2016040683 | |
| | | US20170362330 | |
| | | US10533056 | |
| | | US20140271685 | |
| | | US9567402 | |
| CD46 (MCP, | N/A | WO2003032814 | MacroGenics Inc |
| Membrane | | | |
| cofactor protein) | | | |
| CD46 (MCP, | N/A | US20170081419 | StemCentRx |
| Membrane | | US10017565 | |
| cofactor protein) | | WO2012031273 | |
| | | US9458231 | |
| | | US20130302355 | |

### 2. F Proteins

In some embodiments, the lipid particle comprises one or more paramyxovirus fusion (F) proteins. In some embodiments, the lipid particle contains an exogenous or overexpressed paramyxovirus fusion (F) protein. In some embodiments, the paramyxovirus fusion (F) protein is disposed in the lipid bilayer. In some embodiments, the paramyxovirus fusion (F) protein (e.g., fusogen) facilitates the fusion of the lipid particle to a membrane. In some embodiments, the membrane is a plasma cell membrane. In some embodiments, the paramyxovirus fusion (F) protein binds a binding partner on a target cell surface. In some embodiments, the paramyxovirus fusion (F) protein comprises a protein with a hydrophobic fusion peptide domain.

In some embodiments the paramyxovirus fusion (F) protein is or comprises a Nipah virus protein F, a measles virus F protein, a tupaia paramyxovirus F protein, a paramyxovirus F protein, a Hendra virus F protein, a Henipavirus F protein, a Morbillivirus F protein, a respirovirus F protein, a Sendai virus F protein, a rubulavirus F protein, or an avulavirus F protein.

In some embodiments, the paramyxovirus fusion (F) protein comprises a henipavirus F protein molecule or biologically active portion thereof. In some embodiments, the Henipavirus F protein is a Hendra (HeV) virus F protein, a Nipah (NiV) virus F-protein, a Cedar (CedPV) virus F protein, a M6jiang virus F protein, a Langya virus F protein or a bat Paramyxovirus F protein or a biologically active portion thereof.

Table 4 provides non-limiting examples of F proteins. In some embodiments, the N-terminal hydrophobic fusion peptide domain of the F protein molecule or biologically active portion thereof is exposed on the outside of lipid bilayer.

In some embodiments, the paramyxovirus fusion (F) protein is a variant Nipah F protein (NiV-F). In some embodiments, the variant NiV-F protein exhibits fusogenic activity. In some embodiments, the variant NiV-F facilitates the fusion of the lipid particle (e.g. lentiviral vector) to a membrane. F proteins of henipaviruses, including NiV-F, are encoded as F0 precursors containing a signal peptide (e.g. corresponding to amino acid residues 1-26 of the below). Following cleavage of the signal peptide, the mature F0 (SEQ ID NO:235 lacking the signal peptide, i.e. set forth in SEQ ID NO:256) is transported to the cell surface, then endocytosed and cleaved by cathepsin L (e.g. between amino acids 109-110 of NiV-F corresponding to amino acids set forth in SEQ ID NO:235) into the mature fusogenic subunits F1 (e.g. corresponding to amino acids 110-546 of NiV-F set forth in SEQ ID NO:235) and F2 (e.g. corresponding to amino acid residues 27-109 of NiV-F set forth in SEQ ID NO:235). The F1 and F2 subunits are associated by a disulfide bond and recycled back to the cell surface. The F1 subunit contains the fusion peptide domain located at the N terminus of the F1 subunit (e.g. corresponding to amino acids 110-129 of the below e.g. NiV-F set forth in SEQ ID NO:235) where it is able to insert into a cell membrane to drive fusion. In particular cases, fusion activity is blocked by association of the F protein with G protein, until G engages with a target molecule resulting in its disassociation from F and exposure of the fusion peptide to mediate membrane fusion.

Among different henipavirus species, the sequence and activity of the F protein is highly conserved. For examples, the F protein of NiV and HeV viruses share 89% amino acid sequence identity. Further, in some cases, the henipavirus F proteins exhibit compatibility with G proteins from other species to trigger fusion (Brandel-Tretheway et al. Journal of Virology. 2019. 93(13):e00577-19). In some aspects of the provided lipid particles, the F protein is heterologous to the G protein, i.e. the F and G protein or biologically active portions are from different henipavirus species. For example, the F protein is from Hendra virus and the G protein is from Nipah virus. In other aspects, the F protein can be a chimeric F protein containing regions of F proteins from different species of Henipavirus. In some embodiments, switching a region of amino acid residues of the F protein from one species of Henipavirus to another can result in fusion to the G protein of the species comprising the amino acid insertion. (Brandel-Tretheway et al. 2019). In some cases, the chimeric F protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus. F protein sequences disclosed herein are predominantly disclosed as expressed sequences including an N-terminal signal sequence. As such N-terminal signal sequences are commonly cleaved co- or post-translationally, the mature protein sequences for all F protein sequences disclosed herein are also contemplated as lacking the N-terminal signal sequence.

TABLE 4
Non-limiting Examples of F Proteins

| | | | | | SEQ ID |
|---|---|---|---|---|---|
| | Nucleo- | | | | NO |
| | tides | Full | | | (without |
| Genbank | of | Gene | | | signal |
| ID | CDS | Name | Sequence | SEQ ID | sequence) |
| AF017149 | 6618- | gb:AF017149\| | MATQEVRLKCLLCGIIVLVLSLEGLGILH | 234 | 255 |
| | 8258 | Organism: | YEKLSKIGLVKGITRKYKIKSNPLTKDIVI | | |
| | | Hendra | KMIPNVSNVSKCTGTVMENYKSRLTGILS | | |
| | | virus\| | PIKGAIELYNNNTHDLVGDVKLAGVVMA | | |
| | | Strain | GIAIGIATAAQITAGVALYEAMKNADNIN | | |
| | | Name: | KLKSSIESTNEAVVKLQETAEKTVYVLTA | | |
| | | UNKNOWN- | LQDYINTNLVPTIDQISCKQTELALDLALS | | |
| | | AF017149\| | KYLSDLLFVFGPNLQDPVSNSMTIQAISQ | | |
| | | Protein | AFGGNYETLLRTLGYATEDFDDLLESDSI | | |
| | | Name: | AGQIVYVDLSSYYIIVRVYFPILTEIQQAY | | |
| | | fusion\| | VQELLPVSFNNDNSEWISIVPNFVLIRNTL | | |
| | | Gene | ISNIEVKYCLITKKSVICNQDYATPMTAS | | |
| | | Symbol: | VRECLTGSTDKCPRELVVSSHVPRFALSG | | |
| | | F | GVLFANCISVTCQCQTTGRAISQSGEQTL | | |
| | | | LMIDNTTCTTVVLGNIIISLGKYLGSINYN | | |
| | | | SESIAVGPPVYTDKVDISSQISSMNQSLQQ | | |
| | | | SKDYIKEAQKILDTVNPSLISMLSMIILYV | | |
| | | | LSIAALCIGLITFISFVIVEKKRGNYSRLDD | | |
| | | | RQVRPVSNGDLYYIGT | | |
| Q9IH63 | | Additional | MVVILDKRCYCNLLILILMISECSVGILHY | 235 | 256 |
| | | in | EKLSKIGLVKGVTRKYKIKSNPLTKDIVIK | | |
| | | cluster: | MIPNVSNMSQCTGSVMENYKTRLNGILT | | |
| | | sp\|Q9IH63\| | PIKGALEIYKNNTHDLVGDVRLAGVIMA | | |
| | | FUS_ | GVAIGIATAAQITAGVALYEAMKNADNI | | |
| | | NIPAV | NKLKSSIESTNEAVVKLQETAEKTVYVLT | | |
| | | Fusion | ALQDYINTNLVPTIDKISCKQTELSLDLAL | | |
| | | glycoprotein | SKYLSDLLFVFGPNLQDPVSNSMTIQAIS | | |
| | | F0 | QAFGGNYETLLRTLGYATEDFDDLLESD | | |
| | | OS = Nipah | SITGQIIYVDLSSYYIIVRVYFPILTEIQQA | | |
| | | virus | YIQELLPVSFNNDNSEWISIVPNFILVRNT | | |
| | | | LISNIEIGFCLITKRSVICNQDYATPMTNN | | |
| | | | MRECLTGSTEKCPRELVVSSHVPRFALSN | | |
| | | | GVLFANCISVTCQCQTTGRAISQSGEQTL | | |
| | | | LMIDNTTCPTAVLGNVIISLGKYLGSVNY | | |
| | | | NSEGIAIGPPVFTDKVDISSQISSMNQSLQ | | |
| | | | QSKDYIKEAQRLLDTVNPSLISMLSMIILY | | |
| | | | VLSIASLCIGLITFISFIIVEKKRNTYSRLED | | |
| | | | RRVRPTSSGDLYYIGT | | |
| JQ001776 | 6129- | gb: | MSNKRTTVLIIISYTLFYLNNAAIVGFDFD | 236 | 257 |
| | 8166 | JQ001776: | KLNKIGVVQGRVLNYKIKGDPMTKDLVL | | |
| | | 6129- | KFIPNIVNITECVREPLSRYNETVRRLLLPI | | |
| | | 8166\| | HNMLGLYLNNTNAKMTGLMIAGVIMGG | | |
| | | Organism: | IAIGIATAAQITAGFALYEAKKNTENIQKL | | |
| | | Cedar | TDSIMKTQDSIDKLTDSVGTSILILNKLQT | | |
| | | virus\| | YINNQLVPNLELLSCRQNKIEFDLMLTKY | | |
| | | Strain | LVDLMTVIGPNINNPVNKDMTIQSLSLLF | | |
| | | Name: | DGNYDIMMSELGYTPQDFLDLIESKSITG | | |
| | | CG1a\| | QIIYVDMENLYVVIRTYLPTLIEVPDAQIY | | |
| | | Protein | EFNKITMSSNGGEYLSTIPNFILIRGNYMS | | |
| | | Name: | NIDVATCYMTKASVICNQDYSLPMSQNL | | |
| | | fusion | RSCYQGETEYCPVEAVIASHSPRFALTNG | | |
| | | glycoprotein\| | VIFANCINTICRCQDNGKTITQNINQFVSM | | |
| | | Gene | IDNSTCNDVMVDKFTIKVGKYMGRKDIN | | |
| | | Symbol: | NINIQIGPQIIIDKVDLSNEINKMNQSLKDS | | |
| | | F | IFYLREAKRILDSVNISLISPSVQLFLIIISVL | | |
| | | | SFIILLIIIVYLYCKSKHSYKYNKFIDDPDY | | |
| | | | YNDYKRERINGKASKSNNIYYVGD | | |
| NC_025352 | 5950- | gb: | MALNKNMFSSLFLGYLLVYATTVQSSIH | 237 | 258 |
| | 8712 | NC_025352: | YDSLSKVGVIKGLTYNYKIKGSPSTKLM | | |
| | | 5950- | VVKLIPNIDSVKNCTQKQYDEYKNLVRK | | |
| | | 8712\| | ALEPVKMAIDTMLNNVKSGNNKYRFAG | | |
| | | Organism: | AIMAGVALGVATAATVTAGIALHRSNEN | | |
| | | Mojiang | AQAIANMKSAIQNTNEAVKQLQLANKQT | | |
| | | virus\| | LAVIDTIRGEINNNIIPVINQLSCDTIGLSV | | |
| | | Strain | GIRLTQYYSEIITAFGPALQNPVNTRITIQA | | |
| | | Name: | ISSVFNGNFDELLKIMGYTSGDLYEILHSE | | |
| | | Tongguan 1\| | LIRGNIIDVDVDAGYIALEIEFPNLTLVPN | | |
| | | Protein | AVVQELMPISYNIDGDEWVTLVPRFVLT | | |
| | | Name: | RTTLLSNIDTSRCTITDSSVICDNDYALPM | | |
| | | fusion | SHELIGCLQGDTSKCAREKVVSSYVPKFA | | |
| | | protein\| | LSDGLVYANCLNTICRCMDTDTPISQSLG | | |
| | | Gene | ATVSLLDNKRCSVYQVGDVLISVGSYLG | | |
| | | Symbol: | DGEYNADNVELGPPIVIDKIDIGNQLAGI | | |
| | | F | NQTLQEAEDYIEKSEEFLKGVNPSIITLGS | | |
| | | | MVVLYIFMILIAIVSVIALVLSIKLTVKGN | | |
| | | | VVRQQFTYTQHVPSMENINYVSH | | |
| NC_025256 | 6865- | gb: | MKKKTDNPTISKRGHNHSRGIKSRALLRE | 238 | 259 |
| | 8853 | NC_025256: | TDNYSNGLIVENLVRNCHHPSKNNLNYT | | |
| | | 6865- | KTQKRDSTIPYRVEERKGHYPKIKHLIDK | | |
| | | 8853\| | SYKHIKRGKRRNGHNGNIITIILLLILILKT | | |
| | | Organism: | QMSEGAIHYETLSKIGLIKGITREYKVKG | | |
| | | Bat | TPSSKDIVIKLIPNVTGLNKCTNISMENYK | | |
| | | Paramyxovirus | EQLDKILIPINNIIELYANSTKSAPGNARFA | | |
| | | Eid_hel/ | GVIIAGVALGVAAAAQITAGIALHEARQN | | |
| | | GH-M74a/ | AERINLLKDSISATNNAVAELQEATGGIV | | |
| | | GHA/2009\| | NVITGMQDYINTNLVPQIDKLQCSQIKTA | | |
| | | Strain | LDISLSQYYSEILTVFGPNLQNPVTTSMSI | | |
| | | Name: | QAISQSFGGNIDLLLNLLGYTANDLLDLL | | |
| | | BatPV/ | ESKSITGQITYINLEHYFMVIRVYYPIMTTI | | |
| | | Eid_hel/ | SNAYVQELIKISFNVDGSEWVSLVPSYILI | | |
| | | GH-M74a/ | RNSYLSNIDISECLITKNSVICRHDFAMPM | | |
| | | GHA/2009\| | SYTLKECLTGDTEKCPREAVVTSYVPRFA | | |
| | | Protein | ISGGVIYANCLSTTCQCYQTGKVIAQDGS | | |
| | | Name: | QTLMMIDNQTCSIVRIEEILISTGKYLGSQ | | |
| | | fusion | EYNTMHVSVGNPVFTDKLDITSQISNINQ | | |
| | | protein\| | SIEQSKFYLDKSKAILDKINLNLIGSVPISI | | |
| | | Gene | LFIIAILSLILSIITFVIVMIIVRRYNKYTPLI | | |
| | | Symbol: | NSDPSSRRSTIQDVYIIPNPGEHSIRSAARS | | |
| | | F | IDRDRD | | |

In some embodiments, the F protein or the biologically active portion thereof is a wild-type Nipah virus F (NiV-F) protein or a Hendra virus F protein or is a functionally active variant or biologically active portion thereof. For instance, in some embodiments, the F protein or the biologically active portion thereof is a wild-type NiV-F protein or a functionally active variant or a biologically active portion thereof.

In some embodiments, the F protein has the sequence of amino acids set forth in SEQ ID NO:234, SEQ ID NO:235, SEQ ID NO:236, SEQ ID NO:237, or SEQ ID NO:238, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:234, SEQ ID NO:235, SEQ ID NO:236, SEQ ID NO:237, or SEQ ID NO:238, and retains fusogenic activity in conjunction with a G protein, such as a variant NiV-G as provided herein. In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:234, SEQ ID NO:235, SEQ ID NO:236, SEQ ID NO:237, or SEQ ID NO:238.

In particular embodiments, the F protein has the sequence of amino acids set forth in SEQ ID NO:255, SEQ ID NO:256, SEQ ID NO:257, SEQ ID NO:258, or SEQ ID NO:259, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:255, SEQ ID NO:256, SEQ ID NO:257, SEQ ID NO:258, or SEQ ID NO:259, and retains fusogenic activity in conjunction with a G protein, such as a variant NiV-G as provided herein. In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:255, SEQ ID NO:256, SEQ ID NO:257, SEQ ID NO:258, or SEQ ID NO:259.

Fusogenic activity includes the activity of the paramyxovirus fusion (F) protein in conjunction with a paramyxovirus envelope protein (e.g., G protein or G proteins) to promote or facilitate fusion of two membrane lumens, such as the lumen of the targeted lipid particle having embedded in its lipid bilayer a henipavirus F and at least two G proteins, and a cytoplasm of a target cell, e.g. a cell that contains a surface receptor or molecule that is recognized or bound by the targeted envelope protein. In some embodiments, the F protein and at least one G protein are from the same Henipavirus species (e.g. NiV-G and NiV-F). In some embodiments, the F protein and at least one G protein are from different Henipavirus species (e.g. NiV-G and HeV-F). In particular embodiments, the F protein of the functionally active variant or biologically active portion retains the cleavage site cleaved by cathepsin L (e.g. corresponding to the cleavage site between amino acids 109-110 of SEQ ID NO:235).

Reference to retaining fusogenic activity includes activity (in conjunction with a G protein, such as a variant G protein provided herein) that is between at or about 10% and at or about 150% or more of the level or degree of binding of the corresponding wild-type F protein, such as set forth in SEQ ID NO:234, SEQ ID NO:235, SEQ ID NO:236, SEQ ID NO:237, or SEQ ID NO:238, SEQ ID NO:255, SEQ ID NO:256, SEQ ID NO:257, SEQ ID NO:258, or SEQ ID NO:259 or a cathepsin L cleaved from thereof containing an F1 and F2 subunit. In some embodiments, the fusogenic activity is at least or at least about 10% of the level or degree of fusogenic activity of the corresponding wild-type F protein, such as at least or at least about 15% of the level or degree of fusogenic activity of the corresponding wild-type F protein, such as at least or at least about 20% of the level or degree of fusogenic activity of the corresponding wild-type F protein, such as at least or at least about 25% of the level or degree of fusogenic activity of the corresponding wild-type F protein, such as at least or at least about 30% of the level or degree of fusogenic activity of the corresponding wild-type F protein, such as at least or at least about 35% of the level or degree of fusogenic activity of the corresponding wild-type F protein, such as at least or at least about 40% of the level or degree of fusogenic activity of the corresponding wild-type F protein, such as at least or at least about 45% of the level or degree of fusogenic activity of the corresponding wild-type F protein, such as at least or at least about 50% of the level or degree of fusogenic activity of the corresponding wild-type F protein, such as at least or at least about 55% of the level or degree of fusogenic activity of the corresponding wild-type F protein, such as at least or at least about 60% of the level or degree of fusogenic activity of the corresponding wild-type F protein, such as at least or at least about 65% of the level or degree of fusogenic activity of the corresponding wild-type F protein, such as at least or at least about 70% of the level or degree of fusogenic activity of the corresponding wild-type F protein, such as at least or at least about 75% of the level or degree of fusogenic activity of the corresponding wild-type F protein, such as at least or at least about 80% of the level or degree of fusogenic activity of the corresponding wild-type F protein, such as at least or at least about 85% of the level or degree of fusogenic activity of the corresponding wild-type F protein, such as at least or at least about 90% of the level or degree of fusogenic activity of the corresponding wild-type F protein, such as at least or at least about 95% of the level or degree of fusogenic activity of the corresponding wild-type F protein, such as at least or at least about 100% of the level or degree of fusogenic activity of the corresponding wild-type F protein, or such as at least or at least about 120% of the level or degree of fusogenic activity of the corresponding wild-type F protein.

In some embodiments, the paramyxovirus fusion (F) protein is a mutant F protein that is a functionally active fragment or a biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference F protein sequence. In some embodiments, the reference F protein sequence is the wild-type sequence of an F protein or a biologically active portion thereof. In some embodiments, the mutant F protein or the biologically active portion thereof is a mutant of a wild-type Hendra (HeV) virus F protein, a Nipah (NiV) virus F-protein, a Cedar (CedPV) virus F protein, a Mojiang virus F protein or a bat Paramyxovirus F protein. In some embodiments, the wild-type F protein is encoded by a sequence of nucleotides that encodes any one of SEQ ID NO:234, SEQ ID NO:235, SEQ ID NO:236, SEQ ID NO:237, or SEQ ID NO:238, SEQ ID NO:255, SEQ ID NO:256, SEQ ID NO:257, SEQ ID NO:258, or SEQ ID NO:259 or a cathepsin L cleaved from thereof containing an F1 and F2 subunit.

In some embodiments, the mutant F protein is a biologically active portion that is truncated and lacks up to 22 contiguous amino acid residues at or near the C-terminus of the wild-type F protein, such as a wild-type F protein set forth in any one of SEQ ID NO:234, SEQ ID NO:235, SEQ ID NO:236, SEQ ID NO:237, or SEQ ID NO:238, SEQ ID NO:255, SEQ ID NO:256, SEQ ID NO:257, SEQ ID NO:258, or SEQ ID NO:259. In some embodiments, the mutant F protein is truncated and lacks up to 22 contiguous amino acids, such as up to 21, 20, 19, 18, 17, 16, 15, 14, 13, 12, 11, 10, 9, 8, 7, 6, 5, 4, 3, 2, or 1 contiguous amino acids at the C-terminus of the wild-type F protein.

In some embodiments, the NiV-F, such as a mutant or truncated NiV-F, of a provided lipid particle includes the F0 precursor or a proteolytically cleaved form thereof containing the F1 and F2 subunits, such as resulting following proteolytic cleavage at the cleavage site (e.g. between amino acids corresponding to amino acids between amino acids 109-110 of SEQ ID NO:235) to produce two chains that can be linked by disulfide bond. In some embodiments, the NiV-F, such as wild-type NiV-F or a truncated or mutated NiV-F protein, is produced or encoded as an F₀ precursor which then is able to be proteolytically cleaved to result in an F protein containing the F1 and F2 subunit linked by a disulfide bond. Hence, it is understood that reference to a particular sequence (SEQ ID NO) of a NiV-F herein is typically with reference to the F₀ precursor sequence but also is understood to include the proteolytically cleaved form or sequence thereof containing the two cleaved chains, F1 and F2. For instance, the NiV-F, such as a mutant or truncated NiV-F, contains an F1 subunit corresponding to amino acids 110-546 of NiV-F set forth in SEQ ID NO:235 or truncated or mutant sequence thereof, and an F2 corresponding to amino acid residues 27-109 of NiV-F set forth in SEQ ID NO:235.

In some embodiments, the mutant F protein is a biologically active portion that is truncated and lacks up to 22 contiguous amino acid residues at or near the C-terminus of the wild-type NiV-F protein, such as a wild-type NiV-F protein set forth in SEQ ID NO:235 or SEQ ID NO:256. In some embodiments, the mutant F protein is truncated and lacks up to 22 contiguous amino acids, such as up to 21, 20, 19, 18, 17, 16, 15, 14, 13, 12, 11, 10, 9, 8, 7, 6, 5, 4, 3, 2, or 1 contiguous amino acids at the C-terminus of the wild-type NiV-F protein, such as a wild-type NiV-F protein set forth in SEQ ID NO:235 or SEQ ID NO:256. In some embodiments, the mutant F protein contains an F1 subunit and an F2 subunit in which (1) the F1 subunit is truncated and lacks up to 22 contiguous amino acids at or near the C-terminus of the wild-type F1 subunit, such as lacks up to 22 contiguous amino acids at or near the C-terminus of the wild-type F1 subunit corresponding to amino acids 110-546 of NiV-F set forth in SEQ ID NO:235, and (2) the F2 subunit has the sequence corresponding to amino acid residues 27-109 of NiV-F set forth in SEQ ID NO:235.

In some embodiments, the paramyxovirus fusion (F) protein is a mutant NiV-F protein that is a biologically active portion thereof that comprises a 22 amino acid truncation at or near the C-terminus of the wild-type NiV-F protein (SEQ ID NO:235 or SEQ ID NO:256). In some embodiments, the NiV-F protein is encoded by a nucleotide sequence that encodes the sequence set forth in SEQ ID NO: 226. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 226. In particular embodiments, the variant F protein is a mutant NiV-F protein that has the sequence of amino acids set forth in SEQ ID NO:227. In some embodiments, the NiV-F proteins is encoded by a sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 227. In some embodiments, the F protein molecule or biologically active portion thereof comprises the sequence set forth in SEQ ID NO: 227.

In some embodiments, the mutant F protein contains an F1 subunit and an F2 subunit in which (1) the F1 subunit is set forth as amino acids 110-524 of SEQ ID NO:226, and (2) the F2 subunit is set forth as amino acids 27-109 of SEQ ID NO:226.

In some embodiments, the mutant F protein contains an F1 subunit and an F2 subunit in which (1) the F1 subunit is set forth as amino acids 84-498 of SEQ ID NO:227, and (2) the F2 subunit is set forth as amino acids 1-83 of SEQ ID NO:227.

### B. BaEV Fusogen

In some embodiments, the lipid particles provided herein comprise a gammaretroviral attachment protein that is a Baboon Endogenous Retrovirus (BaEV) envelope glycoprotein. In some embodiments, the lipid particles provided herein comprise a BaEV envelope glycoprotein, a first BaEV envelope glycoprotein, and/or a second BaEV envelope glycoprotein. In some embodiments, the BaEV envelope glycoprotein is not retargeted.

In some embodiments, the lipid particles provided herein comprise a first BaEV envelope glycoprotein, a second BaEV envelope glycoprotein, and a third gam BaEV envelope glycoprotein.

In some embodiments, the lipid particles provided herein comprise a first BaEV envelope glycoprotein, a second BaEV envelope glycoprotein, a third BaEV envelope glycoprotein, and one or more additional BaEV envelope glycoprotein, such as a fourth BaEV envelope glycoprotein, or a fourth and fifth BaEV envelope glycoprotein, or a fourth, fifth, and sixth BaEV envelope glycoprotein, or beyond.

Wild-type BaEV envelope glycoproteins are retroviral envelope proteins containing a C-terminal cytoplasmic tail, a transmembrane domain, and an extracellular domain. Maturation of the precursor protein in the Golgi, which requires the minimal sequence [KR]-X-[KR]-R (wherein X is any amino acid), results in two subunits, the surface unit protein or gp70, and the transmembrane protein p20E. The surface unit protein or gp70 and the transmembrane protein p20E remain associated in a labile interaction that may include a disulfide bond. In wild-type BaEV envelope glycoproteins, fusogenicity is controlled by a short, 17 amino acid sequence termed a fusion inhibitory R peptide, which is localized on the C-terminal of the cytoplasmic tail domain.

In wild-type BaEV envelope glycoproteins, the gp70 mediates receptor binding to the ASCT-2 and ASCT-1 receptors on host cells. In some embodiments, the glycoprotein 70 (g70) subunit or a biologically active portion thereof binds the ASCT-2 and ASCT-1 receptors. In wild-type BaEV envelope glycoproteins, the p20E acts as a class I viral fusion protein. The interaction of the gp70 subunit with a host cell membrane triggers refolding of the p20E and is believed to activate the fusogenic potential by unmasking the fusion peptide.

In some aspects, the fusion inhibitory R peptide harbors the tyrosine endocytosis signal YXXL, and its cleavage by the viral protease is thought to potentiate fusogenic activation through molecular rearrangements in the membrane-spanning domain and the extracellular region of the envelope glycoprotein (Salamango et al (2015) Journal of virology 89(24):12492-12500).

In some embodiments, the BaEV fusogen is a variant BaEV fusogen, such as a BaEV fusogen comprising one or more mutations. In some embodiments, the BaEV fusogen comprises an endogenous protease site, such as is found in the wildtype virus as set forth in SEQ ID NO: 795 at positions 543-560 (e.g., VQAL|VLTQ). In some embodiments, the BaEV envelope protein is mutated as a protease site, such as the protease site between the T (cytoplasmic) and R (inhibitory) domains. In some embodiments, the variant BaEV fusogen comprises a heterologous protease site, such as a protease site from HIV. In some embodiments, the variant BaEV fusogen comprises a heterologous protease site, such as is found in the variant BaEV fusogen as set forth in SEQ ID NO: 797 at positions 543-560 (e.g., SQNY|PIVQ).

In some embodiments, the BaEV envelope protein has a sequence set forth in SEQ ID NO: 797 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NO: 797.

In some embodiments, the BaEV envelope glycoprotein is a truncated BaEV envelope glycoprotein comprising a cytoplasmic tail with a partial fusion inhibitory R peptide relative to the inhibitory R peptide of a wild-type BaEV envelope glycoprotein, wherein the partial fusion inhibitory R peptide comprises at least one amino-terminal amino acid but less than the full length of the inhibitory R peptide of the wild-type BaEV envelope glycoprotein. In some embodiments, the BaEV envelope glycoprotein is modified.

In some embodiments, the truncated BaEV envelope glycoprotein comprise a cytoplasmic tail with a partial fusion inhibitory R peptide relative to a wild-type BaEV envelope glycoprotein, wherein the R peptide contains a contiguous portion of the inhibitory R peptide but lacks the full length R peptide of wild-type BaEV envelope glycoprotein. In some embodiments, the truncated BaEV envelope glycoprotein has a cytoplasmic tail that is composed of a partial inhibitory R peptide with at least one, at least two, or at least three contiguous amino-terminal amino acids of the inhibitory R peptide but less than the full-length R peptide relative to wild-type BaEV envelope glycoprotein. In some embodiments, the truncated BaEV envelope glycoprotein has a cytoplasmic tail that has a partial inhibitory R peptide composed of 1 to 16 contiguous amino-terminal amino acids of the inhibitory R peptide of the wild-type BaEV envelope glycoprotein, such as is composed of 1, 2, 3, 4, 5, 6, 7, 8, 9, 10, 11, 12, 12, 14, 15 or 16 amino-terminal amino acids of the inhibitory R peptide of the wild-type BaEV envelope glycoprotein.

In particular embodiments the partial fusion inhibitory R peptide comprises between 1 and 9 contiguous amino-terminal amino acids, but less than the full length, of the inhibitory R peptide of the wild-type BaEV envelope glycoprotein. In particular embodiments the partial fusion inhibitory R peptide comprises between 2 and 9 contiguous amino-terminal amino acids, but less than the full length, of the inhibitory R peptide of the wild-type BaEV envelope glycoprotein. In particular embodiments the partial fusion inhibitory R peptide comprises between 3 and 9 contiguous amino-terminal amino acids, but less than the full length, of the inhibitory R peptide of the wild-type BaEV envelope glycoprotein. In particular embodiments the partial fusion inhibitory R peptide comprises between 4 and 9 contiguous amino-terminal amino acids, but less than the full length, of the inhibitory R peptide of the wild-type BaEV envelope glycoprotein. In particular embodiments the partial fusion inhibitory R peptide comprises between 5 and 9 contiguous amino-terminal amino acids, but less than the full length, of the inhibitory R peptide of the wild-type BaEV envelope glycoprotein. In particular embodiments the partial fusion inhibitory R peptide comprises between 6 and 9 contiguous amino-terminal amino acids, but less than the full length, of the inhibitory R peptide of the wild-type BaEV envelope glycoprotein. In particular embodiments the partial fusion inhibitory R peptide comprises between 7 and 9 contiguous amino-terminal amino acids, but less than the full length, of the inhibitory R peptide of the wild-type BaEV envelope glycoprotein. In particular embodiments the partial fusion inhibitory R peptide comprises between 8 and 9 contiguous amino-terminal amino acids, but less than the full length, of the inhibitory R peptide of the wild-type BaEV envelope glycoprotein. In particular embodiments the partial fusion inhibitory R peptide comprises 9 contiguous amino-terminal amino acids, but less than the full length, of the inhibitory R peptide of the wild-type BaEV envelope glycoprotein.

In some embodiments, the truncated BaEV envelope glycoprotein contains a cytoplasmic tail that is less than the full length cytoplasmic tail of truncated BaEV envelope glycoprotein due to containing a partial inhibitory R peptide. In some embodiments, the truncated BaEV envelope glycoprotein contains a portion of the full-length cytoplasmic tail set forth in SEQ ID NO: 798. In some embodiment, the truncated BaEV envelope glycoprotein contains a cytoplasmic tail that contains between 18 and 33 contiguous amino-terminal amino acids of SEQ ID NO:4, such as 18, 19, 20, 21, 22, 23, 24, 25, 26, 27, 28, 29, 30, 31, 32 or 33 contiguous amino-terminal amino acids of SEQ ID NO: 798. In some embodiments, the cytoplasmic tail of the truncated BaEV envelope is between 18 and 33 amino acids in length, such as is 18, 19, 20, 21, 22, 23, 24, 25, 26, 27, 28, 29, 30, 31, 32 or 33 amino acids in length.

In particular embodiments, the truncated BaEV envelope glycoprotein comprises a portion of the sequence set forth in SEQ ID NO: 798 that is a functionally active variant or biologically active portion thereof that retains fusogenic activity. In some embodiments, the truncated BaEV envelope glycoprotein lacks up to 16 contiguous amino acids from the C-terminal cytoplasmic tail of SEQ ID NO: 798. In some embodiments, the truncated BaEV envelope glycoprotein lacks from 1 to 16 contiguous amino acids of the C-terminal cytoplasmic tail of SEQ ID NO: 798, such as 1, 2, 3, 4, 5, 6, 7, 8, 9, 10, 11, 12, 13, 14, 15 or 16 contiguous amino acids of the C-terminal cytoplasmic tail of SEQ ID NO: 798. In particular embodiments, the truncated BaEV envelope glycoprotein lacks from 8 to 14 contiguous amino acids of the C-terminal cytoplasmic tail of SEQ ID NO: 798. In some embodiments, the truncated BaEV envelope glycoprotein lacks from 8 to 13 contiguous amino acids of the C-terminal cytoplasmic tail of SEQ ID NO: 798.

In some embodiments, the provided truncated Baboon Endogenous Retrovirus (BaEV) envelope glycoproteins, such as when incorporated into a lipid particle (e.g. lentiviral particle), are composed of two chains that include (i) a glycoprotein 70 (g70) subunit or a biologically active portion thereof, and (ii) a portion of the glycoprotein p20E (p20E) subunit containing the cytoplasmic domain with the partial fusion inhibitory R peptide as described.

In particular embodiments, the provided truncated BaEV envelope glycoproteins, such as when incorporated into a lipid particle (e.g. lentiviral particle), exhibit fusogenic activity, which is mediated by the partial fusion inhibitory R peptide. In some embodiments, the provided truncated BaEV envelope glycoproteins containing a partial fusion inhibitory R peptide retain fusogenic activity of wild-type BaEV envelope glycoprotein. Fusogenic activity includes the ability of the truncated BaEV envelope glycoprotein to promote or facilitate fusion of two membrane lumens, and a cytoplasm of a target cell, e.g. a cell that contains a surface receptor or molecule that is recognized or bound by the truncated BaEV envelope protein. In some embodiments, the truncated BaEV envelope glycoproteins provided herein bind the neutral amino acid transporter receptors ASCT-2 or ASCT-1. The ASCT-2 and ASCT-1 receptors share approximately 57% sequence identity, are differentially expressed in cells (e.g. T and B cells) and are transporters of an overlapping but non-identical set of neutral amino acids (Colmartino et al. (2019) Frontiers in Immunology 10(2873): 1-7; Girard-Gagnepain et al (2014) Blood 124:1221-1231; Levy et al. (2016) Journal of Thrombosis and Haemostasis 14:2478-2492).

Reference to retaining fusogenic activity includes activity that is between 10% and at or about 150% or more of the level or degree of binding of the corresponding wild-type BaEV envelope glycoprotein, such as set forth in SEQ ID NO: 798. In some embodiments, the fusogenic activity of a truncated BaEV envelope glycoprotein is between 50% and 125% of the level or degree of binding of the corresponding wild-type BaEV envelope glycoprotein, such as set forth in SEQ ID NO: 798. In some embodiments, the fusogenic activity of a truncated BaEV envelope glycoprotein is between 80% and 120% of the level or degree of binding of the corresponding wild-type BaEV envelope glycoprotein, such as set forth in SEQ ID NO: 798.

In some embodiments, the fusogenic activity of a truncated BaEV envelope glycoprotein is less than the fusogenic activity of a BaEV envelope glycoprotein that lacks the full inhibitory R peptide (R-Less). For instance, in some embodiments, the fusogenic activity of a truncated BaEV envelope glycoprotein is less than the fusogenic activity of a R-Less BaEV envelope glycoprotein that has a 17 amino acid truncation in the distal C-terminal portion of wild-type BaEV envelope glycoprotein, corresponding to amino acids 529-545, of SEQ ID NO: 798. In some embodiments, an R-Less BaEV envelope glycoprotein has the sequence of amino acids set forth in SEQ ID NO:796. In some embodiments, the BaEV envelope protein has a sequence set forth in SEQ ID NOS: 796 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NOS: 796. In some embodiments, a provided truncated BaEV envelope glycoprotein exhibits 10% to 90% of the fusogenic activity of a R-Less BaEV envelope glycoprotein, such as less than at or about 90%, at or about 85%, at or about 80%, at or about 75%, at or about 70%, at or about 60%, at or about 50%, at or about 40%, at or about 30%, at or about 20% or at or about 10% or less of the fusogenic activity of an R-Less BaEV envelope glycoprotein, e.g. as set forth in SEQ ID NO: 796. In some embodiments, the reduced fusogenic activity avoids high levels of cell fusion during production of provided lipid particles, e.g. lentiviral particles. For instance, incorporation of a truncated BaEV envelope glycoprotein during methods of producing a lipid particle, e.g. lentiviral particle, avoids or reduces high levels of syncytia formation of producer cells during production of the particles.

In particular embodiments, the truncated BaEV envelope glycoprotein contains a cytosolic domain (T domain) with a truncated or partial inhibitory R peptide. In some embodiments, the truncated BaEV envelope glycoprotein contains the extracellular and transmembrane domain of a wildtype BaEV as set forth in SEQ ID NO: 795 with a truncated or partial inhibitory R peptide a partial fusion inhibitory R peptide that contains nine contiguous amino terminal amino acids of the wild type inhibitory R peptide (e.g. R+9). In particular embodiments, the truncated BaEV envelope glycoprotein contains a partial fusion inhibitory R peptide that contains eight contiguous amino terminal amino acids of the wild type inhibitory R peptide (e.g. R+8). In particular embodiments, the truncated BaEV envelope glycoprotein contains a partial fusion inhibitory R peptide that contains seven contiguous amino terminal amino acids of the wild type inhibitory R peptide (e.g. R+7). In particular embodiments, the truncated BaEV envelope glycoprotein contains a partial fusion inhibitory R peptide that contains six contiguous amino terminal amino acids of the wild type inhibitory R peptide (e.g. R+6). In particular embodiments, the truncated BaEV envelope glycoprotein contains a partial fusion inhibitory R peptide that contains five contiguous amino terminal amino acids of the wild type inhibitory R peptide (e.g. R+5). In particular embodiments, the truncated BaEV envelope glycoprotein contains a partial fusion inhibitory R peptide that contains four contiguous amino terminal amino acids of the wild type inhibitory R peptide (e.g. R+4). In particular embodiments, the truncated BaEV envelope glycoprotein contains a partial fusion inhibitory R peptide that contains three contiguous amino terminal amino acids of the wild type inhibitory R peptide (e.g. R+3). In particular embodiments, the truncated BaEV envelope glycoprotein contains a partial fusion inhibitory R peptide that contains two contiguous amino terminal amino acids of the wild type inhibitory R peptide (e.g. R+2). In particular embodiments, the truncated BaEV envelope glycoprotein contains a partial fusion inhibitory R peptide that contains one contiguous amino terminal amino acids of the wild type inhibitory R peptide (e.g. R+1). In particular embodiments, the truncated BaEV envelope glycoprotein contains a partial fusion inhibitory R peptide. In particular embodiments, the truncated BaEV envelope glycoprotein contains a partial fusion inhibitory R peptide. In some embodiments, the truncated BaEV envelope glycoprotein is a two chain form containing the gp70 or a biologically active portion thereof and the portion of the p20E subunit containing the cytoplasmic domain with the partial inhibitory polypeptide. In some embodiments, the truncated BaEV envelope glycoprotein is a two chain form containing the gp70 subunit and the portion of the p20E subunit. In some embodiments, the gp70 subunit and the portion of the p20E are associated via an inter-subunit disulfide bond.

### C. Co-display Proteins

In some embodiments, the lipid particles described herein comprise a co-display protein. In some embodiments, the co-display protein is expressed and/or present on the surface of the lipid particle. In some embodiment, the co-display protein is disposed in the lipid bilayer of the lipid particle.

In some embodiments, the co-display protein increases the potency of the lipid particle as compared to a lipid particle not comprising the co-display protein. In some embodiments, the co-display protein increases specificity of the lipid particle as compared to a lipid particle not comprising the co-display protein. In some embodiments, the co-display protein increases specificity and potency of the lipid particle as compared to a lipid particle not comprising the co-display protein. In some embodiments, the co-display protein increases specificity to the target cell.

In some embodiments, the co-display protein comprises a binding means for specifically interacting with a target molecule expressed on the surface of the target cell (e.g., such as a second target molecule that is the same or different than is targeted by the targeting moiety of the attachment protein). In some embodiments, the co-display protein binds to a different protein than the targeting moiety of the attachment protein (e.g., an attachment protein and/or retargeted attachment protein).

In some embodiments, the co-display protein is selected from the group comprising of a glycoprotein, transmembrane protein, receptor, integrin, antibody or antigen-binding fragment, an engineered binding domain, a nanobody, a DARPin, an Aptamer, an Affimer, an Affibody, a Knottin, an Avimer, a Monobody, an Anticalin, a Fynomer, and a targeting peptide.

In some embodiments, the co-display protein comprises (i) an extracellular domain, (ii) a linker, and (iii) an anchor. In some embodiments, the extracellular domain of the co-display protein is a targeting moiety, such as an antibody or antigen-binding fragment, an engineered binding domain, a nanobody, a DARPin, an Aptamer, an Affimer, an Affibody, a Knottin, an Avimer, a Monobody, an Anticalin, a Fynomer, or a targeting peptide. In some embodiments, the extracellular domain of the co-display protein is a T cell binding domain, such as any as described in Section III.A.1.B.I.

In some embodiments, the linker is a peptide linker, such as a rigid or flexible peptide linker. In particular embodiments, the linker is a flexible peptide linker. In some such embodiments, the linker is 1-20 amino acids, such as 1-20 amino acids predominantly composed of glycine. In some embodiments, the linker is 1-20 amino acids, such as 1-20 amino acids predominantly composed of glycine and serine. In some embodiments, the linker is a flexible peptide linker containing amino acids Glycine and Serine, referred to as GS-linkers. In some embodiments, the peptide linker includes the sequences GS, GGS, GGGGS, GGGGGS or combinations thereof. In some embodiments, the polypeptide linker has the sequence (GGS)n, wherein n is 1 to 10. In some embodiments, the polypeptide linker has the sequence (GGGGS)n, wherein n is 1 to 10. In some embodiments, the polypeptide linker has the sequence (GGGGGS)n, wherein n is 1 to 6. In some embodiments, the polypeptide linker has or comprises the amino acid sequence of SEQ ID NO: 405 (GGGGSGGGGSGGGGS).

In some embodiments, the co-display protein comprises or consists of a nucleic acid sequence as set forth in any of SEQ ID NOs: 626-702, or a sequence having 85%, 86%, 87%, 88%, 89%, 90%, 91%, 92%, 93%, 94%, 95%, 96%, 97%, 98%, or 99% identity to a sequence as set forth in any of SEQ ID NOs: 626-702. In some embodiments, the co-display protein comprises or consists of an amino acid sequence as set forth in any of SEQ ID NOs: 703-779, or a sequence having 85%, 86%, 87%, 88%, 89%, 90%, 91%, 92%, 93%, 94%, 95%, 96%, 97%, 98%, or 99% identity to a sequence as set forth in any of SEQ ID NOs: 703-779.

### 1. Immune Stimulating Proteins

In some embodiments, the co-display protein is an immune stimulating protein. In some embodiments, the co-display protein is an immune stimulating protein that stimulates a T cell activity or function. In some embodiments, an immune stimulating protein may be fused to or incorporated in a protein fusogen or lipid particle envelope attachment protein (e.g., a retargeted attachment protein). In some embodiments, an immune stimulating protein may be incorporated into the viral envelope via fusion with a transmembrane domain. In some embodiments, the immune stimulating protein is a T cell binding agent that targets the lipid particle to a T cell and/or stimulates T cells.

In some embodiments, the immune stimulating protein comprises (i) an extracellular domain, (ii) a linker, and (iii) an anchor. In some embodiments, the extracellular domain of the immune stimulating protein is a T cell binding agent that binds to a T cell stimulatory receptor. In some embodiments, the extracellular domain is an antibody or antibody fragment that binds to a T cell stimulatory receptor. In some embodiments, the extracellular domain is a cytokine, and the immune stimulating protein is a membrane anchored cytokine.

In some embodiments, the immune stimulating protein is a polypeptide capable of binding to CD3. In some embodiments, the polypeptide capable of binding to CD3 is an anti-CD3 antibody, or a fragment thereof that retains the ability to bind to CD3. In some embodiments, the anti-CD3 antibody or fragment thereof is a single chain anti-CD3 antibody, such as but not limited to, an anti-CD3 scFv. In some embodiments, the polypeptide capable of binding to CD3 is anti-CD3scFvFc.

In some embodiments, the anti-CD3 antibody can be an available anti-human CD3 monoclonal antibodies or antibody fragment, including but not limited to UCHT1, OKT-3, HIT3A, TRX4, X35-3, VIT3, BMA030 (BW264/56), CLB-T3/3, CRIS7, YTH12.5, F111409, CLB-T3.4.2, TR-66, WT31, WT32, SPv-T3b, 11D8, XIII-141, XII146, XIII-87, 12F6, T3/RW2-8C8, T3/RW24B6, OKT3D, M-T301, SMC2 and F101.01.

In some embodiments, the immune stimulating protein is a polypeptide capable of binding to CD28. In some embodiments, the polypeptide capable of binding to CD28 is an anti-CD28 antibody, or a fragment thereof that retains the ability to bind to CD28. In other embodiments, the polypeptide capable of binding to CD28 is CD80, CD86, or a functional fragment thereof that is capable of binding CD28 and inducing CD28-mediated activation of Akt, such as an external fragment of CD80. In some aspects herein, an external fragment of CD80 means a fragment that is typically present on the outside of a cell in the normal cellular location of CD80, that retains the ability to bind to CD28. In illustrative embodiments, the anti-CD28 antibody or fragment thereof is a single chain anti-CD28 antibody, such as, but not limited to, an anti-CD28 scFv. In another illustrative embodiment, the polypeptide capable of binding to CD28 is CD80, or a fragment of CD80 such as an external fragment of CD80.

Anti-CD28 antibodies are known in the art and can include, as non-limiting examples, monoclonal antibody 9.3, an IgG2a antibody (Dr. Jeffery Ledbetter, Bristol Myers Squibb Corporation, Seattle, Wash.), monoclonal antibody KOLT-2, an IgG1 antibody, 15E8, an IgG1 antibody, 248.23.2, an IgM antibody and EX5.3D10, an IgG2a antibody.

In some of any of the provided embodiments, an immune stimulating protein includes two polypeptides, a polypeptide capable of binding to CD3 and a polypeptide capable of binding to CD28.

In some embodiments, the polypeptide capable of binding to CD3 or CD28 is an antibody, a single chain monoclonal antibody or an antibody fragment, for example a single chain antibody fragment. Accordingly, the antibody fragment can be, for example, a single chain fragment variable region (scFv), an antibody binding (Fab) fragment of an antibody, or a single domain antibody (VH or VL).

In some embodiments, the immune-stimulating protein is a membrane-bound cytokine. In some aspects, cytokines that are naturally secreted can be engineered as fusion proteins to be membrane-bound. Membrane-bound cytokine fusion polypeptides are included in methods and compositions disclosed herein. In some embodiments, lipid particles (e.g., lentiviral vector particles) have a membrane-bound cytokine fusion polypeptide on their surface that is capable of binding a T cell and/or NK cell and promoting proliferation and/or survival thereof. In some aspects, membrane-bound polypeptides are incorporated into the membranes of lipid particles (e.g., lentiviral vector particles), and when a cell is transduced by the lipid particles (e.g., lentiviral vector particles), the fusion of the particle and host cell membranes results in the polypeptide being bound to the membrane of the transduced cell.

In some embodiments, the cytokine fusion polypeptide includes IL-7, IL-15, or an active fragment thereof. In some aspects, the membrane-bound cytokine fusion polypeptides are typically a cytokine fused to heterologous signal sequence and/or a heterologous membrane attachment sequence. In some embodiments, the heterologous membrane attachment sequence is a GPI anchor attachment sequence. The heterologous GPI anchor attachment sequence can be derived from any known GPI-anchored protein (reviewed in Ferguson M A J, Kinoshita T, Hart G W. Glycosylphosphatidylinositol Anchors. In: Varki A, Cummings R D, Esko J D, et al., editors. Essentials of Glycobiology. 2nd edition. Cold Spring Harbor (N.Y.): Cold Spring Harbor Laboratory Press; 2009. Chapter 11). In some embodiments, the heterologous GPI anchor attachment sequence is the GPI anchor attachment sequence from CD14, CD16, CD48, CD55 (DAF), CD59, CD80, and CD87. In some embodiments, the heterologous GPI anchor attachment sequence is derived from CD16. In an illustrative embodiment, the heterologous GPI anchor attachment sequence is derived from Fc receptor FcγRIIIb (CD16b). In some embodiments, the GPI anchor is the GPI anchor of DAF.

### 2. Transduction Adjuvants

In some embodiments, the co-display protein is a transduction adjuvant. In some embodiments, the co-display protein is a transduction adjuvants that increases transduction efficiency of the lipid particle as compared to a lipid particle that does not have a co-display protein comprising a transduction adjuvant.

In some embodiments, the co-display protein binds to a surface molecule on target cells to enhance transduction. In some embodiments, the transduction adjuvant comprises (i) an extracellular domain, (ii) a linker, and (iii) an anchor. In some embodiments, the extracellular domain of the transduction adjuvant binds an integrin binding domain. In some embodiments, the extracellular domain of the transduction adjuvant comprises the extracellular domain of fibronectin, retronectin, or a functional variant thereof.

In some embodiments, the transduction adjuvant is recombinant human fibronectin. In some aspects, fibronectin is a high molecular weight (220-250 kD) component of the extracellular matrix. Fibronectin exists as a dimer and interacts with extracellular matrix components to participate in cell migration, morphology, growth, and adhesion. The recombinant human fibronectin fragment may include three functional domains: a central cell-binding domain, heparin-binding domain II, and a CS1-sequence. The recombinant human fibronectin protein or fragment thereof may be used to increase gene efficiency of viral vector mediated transduction of immune cells by aiding co-localization of target cells and viral vector. In certain embodiments, the recombinant human fibronectin fragment is RetroNectin® (Takara Bio, Japan). In some embodiments, RetroNectin is a recombinant human fibronectin fragment (rFN-CH-296) composed of three functional domains: the cell-binding domain (C-domain), heparin-binding domain (H-domain), and CS-1 sequence. Without wishing to be bound by theory, RetroNectin enhances viral-vector-mediated gene transduction by aiding the co-localization of target cells and virions. Specifically, lipid particles (e.g., viral vector particles) bind RetroNectin via interaction with the H-domain, and target cells bind through the interaction of cell surface integrin receptor VLA-5 and VLA-4 with the fibronectin C-domain and CS-1 site, respectively. In some embodiments, RectroNectin mediated interaction between the lipid particle (e.g., viral vector) and target cell increase the localized concentrations of cells and particles, an effect that is thought to enhance gene transduction.

In some embodiments, the transduction adjuvant is a polycationic transduction adjuvant that is protamine sulfate, a fibronectin-derived transduction adjuvant. In some embodiments, the transduction adjuvant is selected from protamine, POLYBRENE®, and RetroNectin®. In some embodiments, the transduction adjuvant is selected from RetroNectin® or Vectofusin-1@.

### D. Polynucleotides

Provided herein are polynucleotides comprising a nucleic acid sequence encoding a retargeted attachment protein. In some embodiments, the polynucleotides comprise a nucleic acid sequence encoding a G protein, F protein, or biologically active portion thereof. In some embodiments, the polynucleotides further comprise a nucleic acid sequence encoding a binding domain, such as single domain antibody (sdAb) variable domain or biologically active portion thereof. The polynucleotides may include a sequence of nucleotides encoding any of the chimeric attachment described above. The polynucleotide can be a synthetic nucleic acid. Also provided are expression vector containing any of the provided polynucleotides.

In some of any embodiments, expression of natural or synthetic nucleic acids is typically achieved by operably linking a nucleic acid encoding the gene of interest to a promoter and incorporating the construct into an expression vector. In some embodiments, vectors can be suitable for replication and integration in eukaryotes. In some embodiments, cloning vectors contain transcription and translation terminators, initiation sequences, and promoters useful for expression of the desired nucleic acid sequence. In some of any embodiments, a plasmid comprises a promoter suitable for expression in a cell.

In some embodiments, the polynucleotides contain at least one promoter that is operatively linked to control expression of the targeted retargeted attachment protein and/or G protein and/or F protein. For expression of the retargeted attachment protein, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

In some embodiments, additional promoter elements, e.g., enhancers, regulate the frequency of transcriptional initiation. In some embodiments, additional promoter elements are located in the region 30-110 bp upstream of the start site, although a number of promoters have recently been shown to contain functional elements downstream of the start site as well. In some embodiments, spacing between promoter elements frequently is flexible, so that promoter function is preserved when elements are inverted or moved relative to one another. In some embodiments, the thymidine kinase (tk) promoter, the spacing between promoter elements can be increased to 50 bp apart before activity begins to decline. In some embodiments, depending on the promoter, individual elements can function either cooperatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202 and 5,928,906).

In some embodiments, a suitable promoter is the immediate early cytomegalovirus (CMV) promoter sequence. In some embodiments, the promoter sequence is a strong constitutive promoter sequence capable of driving high levels of expression of any polynucleotide sequence operatively linked thereto. In some embodiments, a suitable promoter is Elongation Growth Factor-1a (EF-1 a). In some embodiments, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

In some embodiments, the promoter is an inducible promoter. In some embodiments, the inducible promoter provides a molecular switch capable of turning on expression of the polynucleotide sequence which it is operatively linked when such expression is desired, or turning off the expression when expression is not desired. In some embodiments, inducible promoters comprise metallothionine promoter, a glucocorticoid promoter, a progesterone promoter, and a tetracycline promoter.

In some embodiments, exogenously controlled inducible promoters can be used to regulate expression of the retargeted attachment protein, the G protein, the F protein, and/or an antigen binding domain such as a single domain antibody (sdAb) variable domain. For example, radiation-inducible promoters, heat-inducible promoters, and/or drug-inducible promoters can be used to selectively drive transgene expression in, for example, targeted regions. In such embodiments, the location, duration, and level of transgene expression can be regulated by the administration of the exogenous source of induction.

In some embodiments, expression of the retargeted attachment protein is regulated using a drug-inducible promoter. For example, in some cases, the promoter, enhancer, or transactivator comprises a Lac operator sequence, a tetracycline operator sequence, a galactose operator sequence, a doxycycline operator sequence, a rapamycin operator sequence, a tamoxifen operator sequence, or a hormone-responsive operator sequence, or an analog thereof. In some instances, the inducible promoter comprises a tetracycline response element (TRE). In some embodiments, the inducible promoter comprises an estrogen response element (ERE), which can activate gene expression in the presence of tamoxifen. In some instances, a drug-inducible element, such as a TRE, can be combined with a selected promoter to enhance transcription in the presence of drug, such as doxycycline. In some embodiments, the drug-inducible promoter is a small molecule-inducible promoter.

Any of the provided polynucleotides can be modified to remove CpG motifs and/or to optimize codons for translation in a particular species, such as human, canine, feline, equine, ovine, bovine, etc. species. In some embodiments, the polynucleotides are optimized for human codon usage (i.e., human codon-optimized). In some embodiments, the polynucleotides are modified to remove CpG motifs. In other embodiments, the provided polynucleotides are modified to remove CpG motifs and are codon-optimized, such as human codon-optimized. Methods of codon optimization and CpG motif detection and modification are well-known. Typically, polynucleotide optimization enhances transgene expression, increases transgene stability and preserves the amino acid sequence of the encoded polypeptide.

In order to assess the expression of the targeted envelope protein, the expression vector to be introduced into a cell can also contain either a selectable marker gene or a reporter gene or both to facilitate identification and selection of expressing particles, e.g. viral particles. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000, FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of the desired polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### IV. LIPID PARTICLES AND METHODS OF PRODUCTION

Provided herein is a lipid particle comprising a lipid bilayer, a lumen surrounded by the lipid bilayer and an attachment protein, such as any as described, in which the attachment protein is embedded within the lipid bilayer. In some embodiments, the lipid particle further comprises a co-display protein. In some embodiments, the provided lipid particles preferentially target hematopoietic cells (e.g. T cells), which is mediated by the tropism of the retargeted attachment protein, such as a G protein. In some embodiments, the lipid particle may additionally contain an exogenous agent (e.g. therapeutic agent) for delivery to a cell. In some embodiments, a lipid particle is introduced to a cell in the subject. Also provided are methods of delivering any of the provided lipid particles to a cell.

In some embodiments, the provided lipid particles exhibit fusogenic activity, which is mediated by the retargeted attachment protein, such as a G protein and/or any of the provided F proteins that facilitates merger or fusion of the two lumens of the lipid particle and the target cell membranes. Thus, among provided lipid particles are fusosomes. In some embodiments, the fusosome comprises a naturally derived bilayer of amphipathic lipids with the attachment protein as a fusogen. In some embodiments, the fusosome comprises (a) a lipid bilayer, (b) a lumen (e.g., comprising cytosol) surrounded by the lipid bilayer; and (c) a fusogen that is exogenous or overexpressed relative to the source cell. In some embodiments, the retargeted attachment protein is disposed in the lipid bilayer. In some embodiments, the fusosome comprises several different types of lipids, e.g., amphipathic lipids, such as phospholipids

In some embodiments, the lipid particle includes a naturally derived bilayer of amphipathic lipids that encloses lumen or cavity. In some embodiments, the lipid particle comprises a lipid bilayer as the outermost surface. In some embodiments, the lipid bilayer encloses a lumen. In some embodiments, the lumen is aqueous. In some embodiments, the lumen is in contact with the hydrophilic head groups on the interior of the lipid bilayer. In some embodiments, the lumen is a cytosol. In some embodiments, the cytosol contains cellular components present in a source cell. In some embodiments, the cytosol does not contain components present in a source cell. In some embodiments, the lumen is a cavity. In some embodiments, the cavity contains an aqueous environment. In some embodiments, the cavity does not contain an aqueous environment.

In some aspects, the lipid bilayer is derived from a source cell during a process to produce a lipid-containing particle. Exemplary methods for producing lipid-containing particles are provided in Section IV. In some embodiments, the lipid bilayer includes membrane components of the cell from which the lipid bilayer is produced, e.g., phospholipids, membrane proteins, etc. In some embodiments, the lipid bilayer includes a cytosol that includes components found in the cell from which the microvesicle is produced, e.g., solutes, proteins, nucleic acids, etc., but not all of the components of a cell, e.g., they lack a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In some embodiments, the lipid bilayer is a viral envelope. In some embodiments, the viral envelope is obtained from a source cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with retargeted attachment proteins that are viral proteins, including viral glycoproteins as described herein such as a G protein and, in some aspects, also a F protein.

In other aspects, the lipid bilayer includes synthetic lipid complex. In some embodiments, the synthetic lipid complex is a liposome. In some embodiments, the lipid bilayer is a vesicular structure characterized by a phospholipid bilayer membrane and an inner aqueous medium. In some embodiments, the lipid bilayer has multiple lipid layers separated by aqueous medium. In some embodiments, the lipid bilayer forms spontaneously when phospholipids are suspended in an excess of aqueous solution. In some examples, the lipid components undergo self-rearrangement before the formation of closed structures and entrap water and dissolved solutes between the lipid bilayers.

In some embodiments, a targeted envelope protein and fusogen, such as any described above including any that are exogenous or overexpressed relative to the source cell, is disposed in the lipid bilayer.

In some embodiments, the lipid particle comprises several different types of lipids. In some embodiments, the lipids are amphipathic lipids. In some embodiments, the amphipathic lipids are phospholipids. In some embodiments, the phospholipids comprise phosphatidylcholine, phosphatidylethanolamine, phosphatidylinositol, and phosphatidylserine. In some embodiments, the lipids comprise phospholipids such as phosphocholines and phosphoinositols. In some embodiments, the lipids comprise DMPC, DOPC, and DSPC.

In some embodiments, the bilayer may be comprised of one or more lipids of the same or different type. In some embodiments, the source cell comprises a cell selected from HEK293 cells, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

In some embodiments, the lipid particle can be a viral particle, a virus-like particle, a nanoparticle, a vesicle, an exosome, a dendrimer, a lentivirus, a viral vector, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a lentiviral vector, a viral based particle, a virus like particle (VLP) or a cell based particle.

In particular embodiments, the lipid particle is virally derived. In some embodiments, the lipid particle can be a viral-based particle, such as a viral vector particle (e.g. lentiviral vector particle) or a virus-like particle (e.g. a lentiviral-like particle). In some embodiments, the lipid bilayer is a viral envelope. In some embodiments, the viral envelope is obtained from a host cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with viral proteins, including viral glycoproteins.

In particular embodiments, the lipid particle is not virally derived. In some embodiments, the lipid particle can be a nanoparticle, a vesicle, an exosome, a dendrimer, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a cell derived particle.

In some embodiments, the lipid bilayer includes membrane components of the host cell from which the lipid bilayer is derived, e.g., phospholipids, membrane proteins, etc. In some embodiments, the lipid bilayer includes a cytosol that includes components found in the cell from which the vehicle is derived, e.g., solutes, proteins, nucleic acids, etc., but not all of the components of a cell, e.g., lacking a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In particular embodiments, an exogenous agent, such as a polynucleotide or polypeptide, is encapsulated within the lumen of a lipid particle. Embodiments of provided lipid particles may have various properties that facilitate delivery of a payload, such as, e.g., a desired transgene or exogenous agent, to a target cell. The exogenous agent may be a polynucleotide or a polypeptide. In some embodiments, a lipid particle provided herein is administered to a subject, e.g., a mammal, e.g., a human. In such embodiments, the subject may be at risk of, may have a symptom of, or may be diagnosed with or identified as having, a particular disease or condition. In one embodiment, the subject has cancer. In one embodiment, the subject has an infectious disease. In some embodiments, the lipid particle contains nucleic acid sequences (polynucleotide) encoding an exogenous agent or a polypeptide exogenous agent for treating the disease or condition.

The lipid particles can include spherical particles or can include particles of elongated or irregular shape.

In some embodiments, a composition of particles can be assessed for one or more features related to their size, including diameter, range of variation thereof above and below an average (mean) or median value of the diameter, coefficient of variation, polydispersity index or other measure of size of particles in a composition. Various methods for particle characterization can be used, including, but not limited to, laser diffraction, dynamic light scattering (DLS; also known as photon correlation spectroscopy) or image analysis, such as microscopy or automated image analysis.

In some embodiments, the provided lipid particle has a diameter of, or the average (mean) diameter of particles in a composition is, less than about 3 m, less than about 2 m, less than about 1 m, less than about 900 nm, less than about 800 nm, less than about 700 nm, less than about 600 nm, less than about 500 m, less than about 400 nm, less than about 300, less than about 200 nm, less than about 150 nm, less than about 100 nm, less than about 50 nm, or less than about 20 nm. In some embodiments, the lipid particle has a diameter of, or the average (mean) diameter of particles in a composition is, less than about 400 nm. In another embodiment, the lipid particle has a diameter of, or the average (mean) diameter of particles in a composition is, less than about 150 nm. In some embodiments, the lipid particle has a diameter of, or the average (mean) diameter of particles in a composition is, between at or about 2 m and at or about 1 m, between at or about 1 m and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments the median particle diameter in a composition of particles is between at or about 10 nm and at or about 1000 nM, between at or about 25 nm and at or about 500 nm, between at or about 40 nm and at or about 300 nm, between at or about 50 nm and at or about 250 nm, between at or about 60 nm and at or about 225 nm, between at or about 70 nm and at or about 200 nm, between at or about 80 nm and at or about 175 nm, or between at or about 90 nm and at or about 150 nm.

In some embodiments, 90% of the lipid particles in a composition fall within 50% of the median diameter of the lipid particles. In some embodiments, 90% of the lipid particles in a composition fall within 25% of the median diameter of the lipid particles. In some embodiments, 90% of the lipid particles in a composition fall within 20% of the median diameter. In some embodiments, 90% of the lipid particles in a composition fall within 15% of the median diameter of lipid particles. In some embodiments, 90% of the lipid particles in a composition fall within 10% of the median diameter of the lipid particles.

In some embodiments, 75% of the lipid particles in a composition fall within +/−2 or +/−1 St Dev standard deviations (St Dev) of the mean diameter of lipid particles. In some embodiments, 80% of the lipid particles in a composition fall within +/−2 St Dev or +/−1 St Dev of the mean diameter of lipid particles. In some embodiments, 85% of the lipid particles in a composition fall within +/−2 St Dev or +/−1 St Dev of the mean diameter of lipid particles. In some embodiments, 90% of the lipid particles in a composition fall within +/−2 St Dev or +/−1 St Dev of the mean diameter of lipid particles. In some embodiments, 95% of the lipid particles in a composition fall within +/−2 St Dev or +/−1 St Dev of the mean diameter of lipid particles.

In some embodiments, the lipid particles have an average hydrodynamic radius, e.g. as determined by DLS, of about 100 nm to about two microns. In some embodiments, the lipid particles have an average hydrodynamic radius between at or about 2 m and at or about 1 m, between at or about 1 m and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments, the lipid particles have an average geometric radius, e.g. as determined by a multi-angle light scattering, of about 100 nm to about two microns. In some embodiments, the lipid particles have an average geometric radius between at or about 2 m and at or about 1 m, between at or about 1 m and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments, the coefficient of variation (COV) (i.e. standard deviation divided by the mean) of a composition of lipid particles is less than at or about 30%, less than at or about 25%, less than at or about 20%, less than at or about 15%, less than at or about 10% or less than at or about 5%.

In some embodiment, provided compositions of lipid particles are characterized by their polydispersity index, which is a measure of the size distribution of the particles wherein values between 1 (maximum dispersion) and 0 (identical size of all of the particles) are possible. In some embodiments, compositions of lipid particles provided herein have a polydispersity index of between at or about 0.05 and at or about 0.7, between at or about 0.05 and at or about 0.6, between at or about 0.05 and at or about 0.5, between at or about 0.05 and at or about 0.4, between at or about 0.05 and at or about 0.3, between at or about 0.05 and at or about 0.2, between at or about 0.05 and at or about 0.1, between at or about 0.1 and at or about 0.7, between at or about 0.1 and at or about 0.6, between at or about 0.1 and at or about 0.5, between at or about 0.1 and at or about 0.4, between at or about 0.1 and at or about 0.3, between at or about 0.1 and at or about 0.2, between at or about 0.2 and at or about 0.7, between at or about 0.2 and at or about 0.6, between at or about 0.2 and at or about 0.5, between at or about 0.2 and at or about 0.4 between at or about 0.2 and at or about 0.3, between at or about 0.3 and at or about 0.7, between at or about 0.3 and at or about 0.6, between at or about 0.3 and at or about 0.5, between at or about 0.3 and at or about 0.4, between at or about 0.4 and at or about 0.7, between at or about 0.4 and at or about 0.6, between at or about 0.4 and at or about 0.5, between at or about 0.5 and at or about 0.7, between at or about 0.5 and at or about 0.6, or between at or about 0.6 and at or about 0.7. In some embodiments, the polydispersity index is less than at or about 0.05, less than at or about 0.1, less than at or about 0.15, less than at or about 0.2, less than at or about 0.25, less than at or about 0.3, less than at or about 0.4, less than at or about 0.5, less than at or about 0.6 or less than at or about 0.7. Various lipid particles are known, any of which can be generated in accord with the provided embodiments. Non-limiting examples of lipid particles include any as described in, or contain features as described in, International published PCT Application No. WO 2017/095946; WO 2017/095944; WO 2017/095940; WO 2019/157319; WO 2018/208728; WO 2019/113512; WO 2019/161281; WO 2020/102578; WO 2019/222403; WO 2020/014209; WO 2020/102485; WO 2020/102499; WO 2020/102503; WO 2013/148327; WO 2017/182585; WO 2011/058052; or WO 2017/068077, each of which are incorporated by reference in their entirety.

Features of the provided lipid particles are described in the following subsections.

### A. Viral-Based Particles

Provided herein are viral-based particles derived from a virus, including those derived from retroviruses or lentiviruses, containing a retargeted attachment protein, such as described in Section III.A.1.B. In some embodiments, the lipid particle's bilayer of amphipathic lipids is or comprises the viral envelope. In some embodiments, the lipid particle's bilayer of amphipathic lipids is or comprises lipids derived from a producer cell. In some embodiments, the viral envelope may comprise a fusogen, e.g., a fusogen that is endogenous to the virus or a pseudotyped fusogen. In some embodiments, the lipid particle's lumen or cavity comprises a viral nucleic acid, e.g., a retroviral nucleic acid, e.g., a lentiviral nucleic acid. In some embodiments, the viral nucleic acid may be a viral genome. In some embodiments, the lipid particle further comprises one or more viral non-structural proteins, e.g., in its cavity or lumen. In some embodiments, the viral-based particle is or comprises a virus-like particle (VLP). In some embodiments, the VLP does not comprise any viral genetic material. In some embodiments, the viral-based particle does not contain any virally derived nucleic acids or viral proteins, such as viral structural proteins.

Biological methods for introducing an exogenous agent to a host cell include the use of DNA and RNA vectors. DNA and RNA vectors can also be used to house and deliver polynucleotides and polypeptides. Viral vectors and virus like particles, and especially retroviral vectors, have become the most widely used method for inserting genes into mammalian, e.g., human cells. Other viral vectors and virus like particles can be derived from lentivirus, poxviruses, herpes simplex virus I, adenoviruses and adeno-associated viruses, and the like. See, for example, U.S. Pat. Nos. 5,350,674 and 5,585,362. Methods for producing cells comprising vectors and/or exogenous acids are well-known in the art. See, for example, Sambrook et al., 2001, Molecular Cloning: A Laboratory Manual, Cold Spring Harbor Laboratory, New York.

In some embodiments, the viral particles or virus-like particles bilayer of amphipathic lipids is or comprises lipids derived from an infected host cell. In some embodiments, the lipid bilayer is a viral envelope. In some embodiments, the viral particles or virus-like particles envelope is obtained from a host cell. In some embodiments, the viral particles or virus-like particles envelope is obtained by the viral capsid from the source cell plasma membrane. In some embodiments, the lipid bilayer is obtained from a membrane other than the plasma membrane of a host cell. In some embodiments, the viral particles or virus-like particles envelope lipid bilayer is embedded with retargeted attachment protein or proteins that are viral proteins, including viral glycoproteins, including a G protein and/or an F protein.

In some embodiments, one or more transducing units of viral particles or virus-like particles, e.g. retroviral particles or retroviral-like particles, are administered to the subject. In some embodiments, at least 1, 10, 100, 1000, 104, 105, 106, 107, 108, 109, 1010, 1011, 1012, 1013, or 1014, transducing units per kg are administered to the subject. In some embodiments at least 1, 10, 100, 1000, 104, 105, 106, 107, 108, 109, 1010, 1011, 1012, 1013, or 1014, transducing units per target cell per ml of blood are administered to the subject.

### 1. Viral Vector Particles

In some embodiments, the lipid particle is or comprises a virus or a viral vector, e.g., a retrovirus or retroviral vector, e.g., a lentivirus or lentiviral vector. In some embodiments, the virus or viral vector is recombinant. For instance, the viral particle may be referred to as a recombinant virus and/or a recombinant viral vector, which are used interchangeably. In some embodiments, the lipid particle is a recombinant lentivirus vector particle.

In some embodiments, a lipid particle comprises a lipid bilayer comprising a retroviral vector comprising an envelope. For instance, in some embodiments, the bilayer of amphipathic lipids is or comprises the viral envelope. The viral envelope may comprise a fusogen, e.g., retargeted attachment protein fusogen, that is endogenous to the virus or is a pseudotyped fusogen. In some embodiments, the viral vector's lumen or cavity comprises a viral nucleic acid, e.g., a retroviral nucleic acid, e.g., a lentiviral nucleic acid. The viral nucleic acid may be a viral genome. In some embodiments, the viral vector may further comprises one or more viral non-structural proteins, e.g., in its cavity or lumen. In some embodiments, the virus based vector particles are lentivirus. In some embodiments, the lentiviral vector particle is Human Immunodeficiency Virus-1 (HIV-1).

In some aspects, the viral vector particle is limited in the number of polynucleotides that can be packaged. In some embodiments, nucleotides encoding polypeptides to be packaged can be modified such that they retain functional activity with fewer nucleotides in the coding region than that which encodes for the wild-type peptide. Such modifications can include truncations, or other deletions. In some embodiments, more than one polypeptide can be expressed from the same promoter, such that they are fusion polypeptides. In some embodiments, the insert size to be packaged (i.e., viral genome, or portions thereof; or heterologous polynucleotides as described) can be between 500-1000, 1000-2000, 2000-3000, 3000-4000, 4000-5000, 5000-6000, 6000-7000, or 7000-8000 nucleotides in length. In some embodiments, the insert can be over 8000 nucleotides, such as 9000, 10,000, or 11,000 nucleotides in length.

In some embodiments, the viral vector particle, such as retroviral vector particle, comprises one or more of gag polyprotein, polymerase (e.g., pol), integrase (e.g., a functional or non-functional variant), protease, and a fusogen. In some embodiments, the lipid particle further comprises rev. In some embodiments, one or more of the aforesaid proteins are encoded in the retroviral genome (i.e., the insert as described above), and in some embodiments, one or more of the aforesaid proteins are provided in trans, e.g., by a helper cell, helper virus, or helper plasmid. In some embodiments, the lipid particle nucleic acid (e.g., retroviral nucleic acid) comprises one or more of the following nucleic acid sequences: 5′ LTR (e.g., comprising U5 and lacking a functional U3 domain), Psi packaging element (Psi, Ψ), Central polypurine tract (cPPT) Promoter operatively linked to the payload gene, payload gene (optionally comprising an intron before the open reading frame), Poly A tail sequence, WPRE, and 3′ LTR (e.g., comprising U5 and lacking a functional U3). In some embodiments, the lipid particle nucleic acid further comprises a retroviral cis-acting RNA packaging element, and a cPPT/CTS element. In some embodiments the lipid particle nucleic acid further comprises one or more insulator element. In some embodiments, the recognition sites are situated between the poly A tail sequence and the WPRE.

In some embodiments, the lipid particle comprises supramolecular complexes formed by viral proteins that self-assemble into capsids. In some embodiments, the lipid particle is a viral particle derived from viral capsids. In some embodiments, the lipid particle is a viral particle derived from viral nucleocapsids. In some embodiments, the lipid particle comprises nucleocapsid-derived that retain the property of packaging nucleic acids.

In some embodiments, the lipid particle packages nucleic acids from host cells carrying one or more viral nucleic acids (e.g. retroviral nucleic acids) during the expression process. In some embodiments, the nucleic acids do not encode any genes involved in virus replication. In particular embodiments, the lipid particle is a virus-based particle, e.g. retrovirus particle such as a lentivirus particle, that is replication defective.

In some cases, the lipid particle is a viral particle that is morphologically indistinguishable from the wild type infectious virus. In some embodiments, the viral particle presents the entire viral proteome as an antigen. In some embodiments, the viral particle presents only a portion of the proteome as an antigen.

In some embodiments, the retroviral nucleic acid comprises one or more of (e.g., all of): a 5′ promoter (e.g., to control expression of the entire packaged RNA), a 5′ LTR (e.g., that includes R (polyadenylation tail signal) and/or U5 which includes a primer activation signal), a primer binding site, a psi packaging signal, a RRE element for nuclear export, a promoter directly upstream of the transgene to control transgene expression, a transgene (or other exogenous agent element), a polypurine tract, and a 3′ LTR (e.g., that includes a mutated U3, a R, and U5). In some embodiments, the retroviral nucleic acid further comprises one or more of a cPPT, a WPRE, and/or an insulator element.

A retrovirus typically replicates by reverse transcription of its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

In some embodiments the retrovirus is a Gammaretrovirus. In some embodiments the retrovirus is an Epsilonretrovirus. In some embodiments the retrovirus is an Alpharetrovirus. In some embodiments the retrovirus is a Betaretrovirus. In some embodiments the retrovirus is a Deltaretrovirus. In some embodiments the retrovirus is a Lentivirus. In some embodiments the retrovirus is a Spumaretrovirus. In some embodiments the retrovirus is an endogenous retrovirus.

Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) are used.

A viral vector can comprise a nucleic acid molecule (e.g., a transfer plasmid) that includes virus-derived nucleic acid elements that typically facilitate transfer of a nucleic acid molecule (e.g. including nucleic acid encoding an exogenous agent) or integration into the genome of a cell or to a viral particle that mediates nucleic acid transfer. Viral vector particles will typically include various viral components and sometimes also host cell components in addition to nucleic acid(s). A viral vector can comprise a virus or viral particle capable of transferring a nucleic acid into a cell (e.g. nucleic acid encoding an exogenous agent), or to the transferred nucleic acid (e.g., as naked DNA). Viral vectors and transfer plasmids can comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some vectors described herein, at least part of one or more protein coding regions that contribute to or are essential for replication may be absent compared to the corresponding wild-type virus. This makes the viral vector replication-defective. In some embodiments, the vector is capable of transducing a target non-dividing host cell and/or integrating its genome into a host genome.

The structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). The LTRs are involved in proviral integration and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are typically similar (e.g., identical) sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

For the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. Some retroviruses comprise any one or more of the following genes that code for proteins that are involved in the regulation of gene expression: tot, rev, tax and rex. With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction promotes infection, e.g., by fusion of the viral membrane with the cell membrane.

In a replication-defective retroviral vector genome gag, pol and env may be absent or not functional. The R regions at both ends of the RNA are typically repeated sequences. U5 and U3 represent unique sequences at the 5′ and 3′ ends of the RNA genome respectively.

Retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. Proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11). The mechanisms of action of these two proteins are thought to be broadly similar to the analogous mechanisms in the primate viruses. In addition, an EIAV protein, Ttm, has been identified that is encoded by the first exon of tat spliced to the env coding sequence at the start of the transmembrane protein.

In addition to protease, reverse transcriptase and integrase, non-primate lentiviruses contain a fourth pol gene product which codes for a dUTPase. This may play a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. Infection of the target cell can comprise reverse transcription and integration into the target cell genome. The RLV typically carries non-viral coding sequences which are to be delivered by the vector to the target cell, such as nucleic acid encoding an exogenous agent as described herein. In embodiments, an RLV is incapable of independent replication to produce infectious retroviral particles within the target cell. Usually the RLV lacks a functional gag-pol and/or env gene and/or other genes involved in replication. The vector may be configured as a split-intron vector, e.g., as described in PCT patent application WO 99/15683, which is herein incorporated by reference in its entirety.

In some embodiments, the lentiviral vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

A minimal lentiviral genome may comprise, e.g., (5′)R-U5-one or more first nucleotide sequences-U3-R(3′)⋅ However, the plasmid vector used to produce the lentiviral genome within a source cell can also include transcriptional regulatory control sequences operably linked to the lentiviral genome to direct transcription of the genome in a source cell. These regulatory sequences may comprise the natural sequences associated with the transcribed retroviral sequence, e.g., the 5′ U3 region, or they may comprise a heterologous promoter such as another viral promoter, for example the CMV promoter. Some lentiviral genomes comprise additional sequences to promote efficient virus production. For example, in the case of HIV, rev and RRE sequences may be included. Alternatively or in combination, codon optimization may be used, e.g., the gene encoding the exogenous agent may be codon optimized, e.g., as described in WO 01/79518, which is herein incorporated by reference in its entirety. Alternative sequences which perform a similar or the same function as the rev/RRE system may also be used. For example, a functional analogue of the rev/RRE system is found in the Mason Pfizer monkey virus. This is known as CTE and comprises an RRE-type sequence in the genome which is believed to interact with a factor in the infected cell. The cellular factor can be thought of as a rev analogue. Thus, CTE may be used as an alternative to the rev/RRE system. In addition, the Rex protein of HTLV-I can functionally replace the Rev protein of HIV-I. Rev and Rex have similar effects to IRE-BP.

In some embodiments, a retroviral nucleic acid (e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non dividing cells.

The deletion of additional genes may permit vectors to be produced without the genes associated with disease in lentiviral (e.g. HIV) infections. In particular, tat is associated with disease. Secondly, the deletion of additional genes permits the vector to package more heterologous DNA. Thirdly, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. Thus, the concentration of free dNTPs in the cytoplasm increases as Vpx degrades SAMHD1 and reverse transcription activity is increased, thus facilitating reverse transcription of the retroviral genome and integration into the target cell genome.

Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. An additional description of codon optimization is found, e.g., in WO 99/41397, which is herein incorporated by reference in its entirety.

Many viruses, including HIV and other lentiviruses, use a large number of rare codons and by changing these to correspond to commonly used mammalian codons, increased expression of the packaging components in mammalian producer cells can be achieved.

In some embodiments, codon optimization has a number of other advantages. In some embodiments, by virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

In some embodiments, derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

In some embodiments, due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

It is within the level of a skilled artisan to empirically determine appropriate codon optimization of viral sequences. The strategy for codon optimized sequences, including gag-pol sequences, can be used in relation to any retrovirus, e.g., EIAV, FIV, BIV, CAEV, VMR, SIV, HIV-I and HIV-2. In addition this method can be used to increase expression of genes from HTLV-I, HTLV-2, HFV, HSRV and human endogenous retroviruses (HERV), MLV and other retroviruses.

In embodiments, the retroviral vector comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

In some embodiments, the retroviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

In some embodiments, the gag and pol coding sequences are generally organized as the Gag-Pol Precursor in native lentivirus. The gag sequence codes for a 55-kD Gag precursor protein, also called p55. The p55 is cleaved by the virally encoded protease (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

In some embodiments, the lentiviral vector is integration-deficient. In some embodiments, the pol is integrase deficient, such as by encoding due to mutations in the integrase gene. For example, the pol coding sequence can contain an inactivating mutation in the integrase, such as by mutation of one or more of amino acids involved in catalytic activity, i.e. mutation of one or more of aspartic 64, aspartic acid 116 and/or glutamic acid 152. In some embodiments, the integrase mutation is a D64V mutation. In some embodiments, the mutation in the integrase allows for packaging of viral RNA into a lentivirus. In some embodiments, the mutation in the integrase allows for packaging of viral proteins into a lentivirus. In some embodiments, the mutation in the integrase reduces the possibility of insertional mutagenesis. In some embodiments, the mutation in the integrase decreases the possibility of generating replication-competent recombinants (RCRs) (Wanisch et al. 2009. Mol Ther. 1798):1316-1332). In some embodiments, native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In some embodiments, the retroviral nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS1, (ii) contains two nucleotide insertion that results in frame shift and premature termination, and/or (iii) does not include INS2, INS3, and INS4 inhibitory sequences of gag.

In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

According to certain specific embodiments, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. A variety of lentiviral vectors are described in Naldini et ah, (1996a, 1996b, and 1998); Zufferey et al., (1997); Dull et al., 1998, U.S. Pat. Nos. 6,013,516; and 5,994,136, many of which may be adapted to produce a retroviral nucleic acid.

At each end of the provirus, long terminal repeats (LTRs) are typically found. An LTR typically comprises a domain located at the ends of retroviral nucleic acid which, in their natural sequence context, are direct repeats and contain U3, R and U5 regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The U5 region is typically the sequence between the primer binding site and the R region and can contain the polyadenylation sequence. The R (repeat) region can be flanked by the U3 and U5 regions. The LTR is typically composed of U3, R and U5 regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

In some embodiments, a packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi [Ψ] sequence) for encapsidation of the viral genome.

In various embodiments, retroviral nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. In some aspects, provided herein is a replication incompetent (also referred to herein as replication defective) vector particle, that cannot participate in replication in the absence of the packaging cell (i.e., viral vector particles are not produced from the transduced cell). In some aspects, this is because the right (3′) LTR U3 region can be used as a template for the left (5′) LTR U3 region during viral replication and, thus, absence of the U3 enhancer-promoter inhibits viral replication. In embodiments, the 3′ LTR is modified such that the U5 region is removed, altered, or replaced, for example, with an exogenous poly(A) sequence The 3′ LTR, the 5′ LTR, or both 3′ and 5′ LTRs, may be modified LTRs. Other modifications to the viral vector, i.e., retroviral or lentiviral vector, to render said vector replication incompetent are known in the art.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

The R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

The retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et ah, 2000, Cell, 101:173, which are herein incorporated by reference in their entireties. During HIV-1 reverse transcription, central initiation of the plus-strand DNA at the central polypurine tract (cPPT) and central termination at the central termination sequence (CTS) can lead to the formation of a three-stranded DNA structure: the HIV-1 central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-L

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE), which are herein incorporated by reference in their entireties. Generally, the RNA export element is placed within the 3′ UTR of a gene, and can be inserted as one or multiple copies.

In some embodiments, expression of heterologous sequences (e.g. nucleic acid encoding an exogenous agent) in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE

In some embodiments, a retroviral nucleic acid described herein lacks or does not comprise a posttranscriptional regulatory element such as a WPRE or HPRE.

Elements directing the termination and polyadenylation of the heterologous nucleic acid transcripts may be included, e.g., to increases expression of the exogenous agent. Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATT AAA, AGTAAA, a bovine growth hormone polyA sequence (BGHpA), a rabbit b-globin polyA sequence (rPgpA), or another suitable heterologous or endogenous polyA sequence.

In some embodiments, a retroviral or lentiviral vector further comprises one or more insulator elements, e.g., an insulator element described herein.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase exogenous gene expression (e.g., poly (A) sequences), and may optionally comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

### 2. Virus-like Particles

In some embodiments, the viral-based particles are viral-like lipid particles (VLPs) that are derived from virus. In some embodiments, the viral envelope may comprise a fusogen, e.g., a fusogen that is endogenous to the virus or a pseudotyped fusogen, e.g., a retargeted attachment protein such as a G or F protein described in Section III.A.1. The VLPs include those derived from retroviruses or lentiviruses. While VLPs mimic native virion structure, they lack the viral genomic information necessary for independent replication within a host cell. Therefore, in some aspects, VLPs are non-infectious. In particular embodiments, a VLP does not contain a viral genome. In some embodiments, the VLP's bilayer of amphipathic lipids is or comprises the viral envelope. In some embodiments, the lipid particle's bilayer of amphipathic lipids is or comprises lipids derived from a cell. In some embodiments, a VLP contains at least one type of structural protein from a virus. In most cases this protein will form a proteinaceous capsid. In some cases the capsid will also be enveloped in a lipid bilayer originating from the cell from which the assembled VLP has been released (e.g. VLPs comprising a human immunodeficiency virus structural protein such as GAG). In some embodiments, the VLP further comprises a targeting moiety as an envelope protein within the lipid bilayer.

In some embodiments, the vector vehicle particle comprises supramolecular complexes formed by viral proteins that self-assemble into capsids. In some embodiments, the vector vehicle particle is a virus-like particle derived from viral capsid proteins. In some embodiments, the vector vehicle particle is a virus-like particle derived from viral nucleocapsid proteins. In some embodiments, the vector vehicle particle comprises nucleocapsid-derived proteins that retain the property of packaging nucleic acids. In some embodiments, the viral-based particles, such as virus-like particles comprises only viral structural glycoproteins among proteins from the viral genome. In some embodiments, the vector vehicle particle does not contain a viral genome.

In some embodiments, the vector vehicle particle packages nucleic acids from host cells during the expression process, such as a nucleic acid encoding an exogenous agent. In some embodiments, the nucleic acids do not encode any genes involved in virus replication. In particular embodiments, the vector vehicle particle is a virus-like particle, e.g. retrovirus-like particle such as a lentivirus-like particle, that is replication defective.

In some embodiments, the vector vehicle particle is a virus-like particle which comprises a sequence that is devoid of or lacking viral RNA may be the result of removing or eliminating the viral RNA from the sequence. In some embodiments, this may be achieved by using an endogenous packaging signal binding site on gag. In some embodiments, the endogenous packaging signal binding site is on pol. In some embodiments, the RNA which is to be delivered will contain a cognate packaging signal. In some embodiments, a heterologous binding domain (which is heterologous to gag) located on the RNA to be delivered, and a cognate binding site located on gag or pol, can be used to ensure packaging of the RNA to be delivered. In some embodiments, the heterologous sequence can be non-viral or it can be viral, in which case it may be derived from a different virus. In some embodiments, the vector particles can be used to deliver therapeutic RNA, in which case functional integrase and/or reverse transcriptase is not required. In some embodiments, the vector particles can also be used to deliver a therapeutic gene of interest, in which case pol is typically included.

In some embodiments, the VLP comprises supramolecular complexes formed by viral proteins that self-assemble into capsids. In some embodiments, the VLP is derived from viral capsids. In some embodiments, the VLP is derived from viral nucleocapsids. In some embodiments, the VLP is nucleocapsid-derived and retains the property of packaging nucleic acids. In some embodiments, the VLP includes only viral structural glycoproteins. In some embodiments, the VLP does not contain a viral genome.

### 3. Methods of Generating Viral-based Particles

Large scale viral particle production is often useful to achieve a desired viral titer. Viral particles can be produced by transfecting a transfer vector into a packaging cell line that comprises viral structural and/or accessory genes, e.g., gag, pol, env, tat, rev, vif, vpr, vpu, vpx, or nef genes or other retroviral genes.

In some embodiments, viral vector particles may be produced in multiple cell culture systems including bacteria, mammalian cell lines, insect cell lines, yeast and plant cells. Methods of producing such viral-based particles can also be found in U.S. Pat. No. 10,316,295, which is incorporated herein by reference. Exemplary methods for producing viral vector particles are described.

In some embodiments, elements for the production of a viral vector, i.e., a recombinant viral vector such as a replication incompetent lentiviral vector, are included in a packaging cell line or are present on a packaging vector. In some embodiments, viral vectors can include packaging elements, rev, gag, and pol, delivered to the packaging cells line via one or more packaging vectors.

In embodiments, the packaging vector is an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. A retroviral, e.g., lentiviral, transfer vector can be introduced into a packaging cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides. In some embodiments, the packaging vector is a packaging plasmid.

Producer cell lines (also called packaging cell lines) include cell lines that do not contain a packaging signal, but do stably or transiently express viral structural proteins and replication enzymes (e.g., gag, pol and env) which can package viral particles. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211 A cells. In embodiments, the packaging cells are 293 cells, 293T cells, or A549 cells.

In some embodiments, a producer cell (i.e., a source cell line) includes a cell line which is capable of producing recombinant retroviral particles, comprising a packaging cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the packaging cells, e.g., by cell lysis, or collection of the supernatant of the cell culture. Optionally, the collected virus particles may be enriched or purified.

In some embodiments, the source cell comprises one or more plasmids coding for viral structural proteins and replication enzymes (e.g., gag, pol and env) which can package viral particles (i.e., a packaging plasmid). In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments a nucleic acid encoding the exogenous agent (e.g., a retroviral nucleic acid encoding the exogenous agent) is also integrated into the source cell genome. In some embodiments a nucleic acid encoding the exogenous agent is maintained episomally. In some embodiments a nucleic acid encoding the exogenous agent is transfected into the source cell that has stably integrated Rev, Gag/Pol, and an envelope protein in the genome. See, e.g., Milani et al. EMBO Molecular Medicine, 2017, which is herein incorporated by reference in its entirety.

In some embodiments, a retroviral nucleic acid described herein is unable to undergo reverse transcription. Such a nucleic acid, in embodiments, is able to transiently express an exogenous agent. The retrovirus or VLP, may comprise a disabled reverse transcriptase protein, or may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid.

Typically, modern retroviral vector systems include viral genomes bearing cis-acting vector sequences for transcription, reverse-transcription, integration, translation and packaging of viral RNA into the viral particles, and (2) producer cells lines which express the trans-acting retroviral gene sequences (e.g., gag, pol and env) needed for production of virus particles. By separating the cis- and trans-acting vector sequences completely, the virus is unable to maintain replication for more than one cycle of infection. Generation of live virus can be avoided by a number of strategies, e.g., by minimizing the overlap between the cis- and trans-acting sequences to avoid recombination.

A virus-like particle (VLP) which comprises a sequence that is devoid of or lacking viral RNA as described in Section IV.A.2 may be the result of removing or eliminating the viral RNA from the sequence. Similar to the viral vector particles disclosed in Section IV.A.1, VLPs contain a viral outer envelope made from the host cell (i.e., producer cell or source cell) lipid-bi layer as well as at least one viral structural protein. In some embodiments, a viral structural protein refers to any viral protein or fragment thereof which contributes to the structure of the viral core or capsid.

Generally, for viral vector particles, expression of the gag precursor protein alone mediates vector assembly and release. In some aspects, gag proteins or fragments thereof have been demonstrated to assemble into structures analogous to viral cores. In one embodiment this may be achieved by using an endogenous packaging signal binding site on gag. Alternatively, the endogenous packaging signal binding site is on pol. In this embodiment, the RNA which is to be delivered will contain a cognate packaging signal. In another embodiment, a heterologous binding domain (which is heterologous to gag) located on the RNA to be delivered, and a cognate binding site located on gag or pol, can be used to ensure packaging of the RNA to be delivered. The heterologous sequence can be non-viral or it can be viral, in which case it may be derived from a different virus. The VLP can be used to deliver therapeutic RNA, in which case functional integrase and/or reverse transcriptase is not required. These VLPs can also be used to deliver a therapeutic gene of interest, in which case pol is typically included.

In an embodiment, gag-pol are altered, and the packaging signal is replaced with a corresponding packaging signal. In this embodiment, the particle can package the RNA with the new packaging signal. The advantage of this approach is that it is possible to package an RNA sequence which is devoid of viral sequence for example, RNAi.

An alternative approach is to rely on over-expression of the RNA to be packaged. In one embodiment the RNA to be packaged is over-expressed in the absence of any RNA containing a packaging signal. This may result in a significant level of therapeutic RNA being packaged, and that this amount is sufficient to transduce a cell and have a biological effect.

In some embodiments, a polynucleotide comprises a nucleotide sequence encoding a viral gag protein or retroviral gag and pol proteins, wherein the gag protein or pol protein comprises a heterologous RNA binding domain capable of recognizing a corresponding sequence in an RNA sequence to facilitate packaging of the RNA sequence into a viral vector particle. In some embodiments, the heterologous RNA binding domain comprises an RNA binding domain derived from a bacteriophage coat protein, a Rev protein, a protein of the U 1 small nuclear ribonucleoprotein particle, a Nova protein, a TF111 A protein, a TIS 11 protein, a trp RNA-binding attenuation protein (TRAP) or a pseudouridine synthase.

In some embodiments, the assembly of a viral based vector particle (i.e., a VLP) is initiated by binding of the core protein to a unique encapsidation sequence within the viral genome (e.g. UTR with stem-loop structure). In some embodiments, the interaction of the core with the encapsidation sequence facilitates oligomerization.

In some embodiments, the source cell for VLP production comprises one or more plasmids coding for viral structural proteins (e.g., gag, pol) which can package viral particles (i.e., a packaging plasmid). In some embodiments, the sequences coding for at least two of the gag and pol precursors are on the same plasmid. In some embodiments, the sequences coding for the gag and pol precursors are on different plasmids. In some embodiments, the sequences coding for the gag and pol precursors have the same expression signal, e.g., promoter. In some embodiments, the sequences coding for the gag and pol precursors have a different expression signal, e.g., different promoters. In some embodiments, expression of the gag and pol precursors is inducible.

In some embodiments, formation of VLPs or any viral-based particle, such as described above in Section IV.A, can be detected by any suitable technique known in the art. Examples of such techniques include, e.g., electron microscopy, dynamic light scattering, selective chromatographic separation and/or density gradient centrifugation.

### B. Exogenous Agent

In some embodiments, the lipid particle as described herein or pharmaceutical composition comprising same described contains an exogenous agent. In some embodiments, the lipid particle or pharmaceutical composition comprising same described herein contains a nucleic acid that encodes an exogenous agent. In some embodiments, the lipid particle contains the exogenous agent. In some embodiments, the lipid particle contains a nucleic acid that encodes an exogenous agent. Reference to the coding sequence of the nucleic acid encoding the exogenous agent also is referred to herein as a payload gene. In some embodiments, the exogenous agent or the nucleic acid encoding the exogenous agent are present in the lumen of the lipid particle.

In some embodiments, the exogenous agent is a protein or a nucleic acid (e.g., a DNA, a chromosome (e.g. a human artificial chromosome), an RNA, e.g., an mRNA or miRNA). In some embodiments, the exogenous agent comprises or encodes a membrane protein. In some embodiments, the exogenous agent comprises or encodes a therapeutic agent. In some embodiments, the therapeutic agent is chosen from one or more of a protein, e.g., an enzyme, a transmembrane protein, a receptor, or an antibody; a nucleic acid, e.g., DNA, a chromosome (e.g. a human artificial chromosome), RNA, mRNA, siRNA, or miRNA; or a small molecule.

In some embodiments, the lipid particle or pharmaceutical composition delivers to a target cell at least 10%, 20%, 30%, 40%, 50%, 60%, 70%, 80%, 90%, 95%, 96%, 97%, 98%, or 99% of the exogenous agent (e.g., an exogenous agent comprising or encoding a therapeutic agent) comprised by the lipid particle. In some embodiments, the lipid particle, e.g., fusosome, that contacts, e.g., fuses, with the target cell(s) delivers to the target cell an average of at least 10%, 20%, 30%, 40%, 50%, 60%, 70%, 80%, 90%, 95%, 96%, 97%, 98%, or 99% of the exogenous agent (e.g., an exogenous agent comprising or encoding a therapeutic agent) comprised by the lipid particles, e.g., fusosomes, that contact, e.g., fuse, with the target cell(s). In some embodiments, the lipid particle composition delivers to a target tissue at least 10%, 20%, 30%, 40%, 50%, 60%, 70%, 80%, 90%, 95%, 96%, 97%, 98%, or 99% of the exogenous agent (e.g., an exogenous agent comprising or encoding a therapeutic agent) comprised by the lipid particle compositions.

In some embodiments, the exogenous agent is not expressed naturally in the cell from which the lipid particle is derived. In some embodiments, the exogenous agent is expressed naturally in the cell from which the lipid particle is derived. In some embodiments, the exogenous agent is loaded into the lipid particle via expression in the cell from which the lipid particle is derived (e.g. expression from DNA or mRNA introduced via transfection, transduction, or electroporation). In some embodiments, the exogenous is expressed from DNA integrated into the genome or maintained episomally. In some embodiments, expression of the exogenous agent is constitutive. In some embodiments, expression of the exogenous agent is induced. In some embodiments, expression of the exogenous agent is induced immediately prior to generating the lipid particle. In some embodiments, expression of the exogenous agent is induced at the same time as expression of the fusogen.

In some embodiments, the exogenous agent is loaded into the lipid particle via electroporation into the lipid particle itself or into the cell from which the lipid particle is derived. In some embodiments, the exogenous agent is loaded into the lipid particle via transfection (e.g., of a DNA or mRNA encoding the exogenous agent) into the lipid particle itself or into the cell from which the lipid particle is derived.

In some embodiments, the exogenous agent may include one or more nucleic acid sequences, one or more polypeptides, a combination of nucleic acid sequences and/or polypeptides, one or more organelles, and any combination thereof. In some embodiments, the exogenous agent may include one or more cellular components. In some embodiments, the exogenous agent includes one or more cytosolic and/or nuclear components.

In some embodiments, the lipid particle contains an exogenous agent that is a nucleic acid or contains a nucleic acid encoding the exogenous agent. In some embodiments, the nucleic acid is operatively linked to a “positive target cell-specific regulatory element” (or positive TCSRE). In some embodiments, the positive TCSRE is a functional nucleic acid sequence. In some embodiments, the positive TCSRE comprises a promoter or enhancer. In some embodiments, the TCSRE is a nucleic acid sequence that increases the level of an exogenous agent in a target cell. In some embodiments, the positive target cell-specific regulatory element comprises a T cell-specific promoter, a T cell-specific enhancer, a T cell-specific splice site, a T cell-specific site extending half-life of an RNA or protein, a T cell-specific mRNA nuclear export promoting site, a T cell-specific translational enhancing site, or a T cell-specific post-translational modification site. In some embodiments, the T cell-specific promoter is a promoter described in Immgen consortium, herein incorporated by reference in its entirety, e.g., the T cell-specific promoter is an IL2RA (CD25), LRRC32, FOXP3, or IKZF2 promoter. In some embodiments, the T cell-specific promoter or enhancer is a promoter or enhancer described in Schmidl et a, Blood. 2014 Apr. 24; 123(17):e68-78., herein incorporated by reference in its entirety. In some embodiments, the T cell-specific promoter is a transcriptionally active fragment of any of the foregoing. In some embodiments, the T-cell specific promoter is a variant having at least 70%, 75%, 80%, 85%, 90%, 95%, 96%, 97%, 98%, or 99% identity to any of the foregoing.

In some embodiments, the lipid particle contains an exogenous agent that is a nucleic acid or contains a nucleic acid encoding the exogenous agent. In some embodiments, the nucleic acid is operatively linked to a “negative target cell-specific regulatory element” (or negative TCSRE). In some embodiments, the negative TCSRE is a functional nucleic acid sequence. In some embodiments, the negative TCSRE is a miRNA recognition site that causes degradation of inhibition of the lipid particle in a non-target cell. In some embodiments, the exogenous agent is operatively linked to a “non-target cell-specific regulatory element” (or NTCSRE). In some embodiments, the NTCSRE comprises a nucleic acid sequence that decreases the level of an exogenous agent in a non-target cell compared to in a target cell. In some embodiments, the NTCSRE comprises a non-target cell-specific miRNA recognition sequence, non-target cell-specific protease recognition site, non-target cell-specific ubiquitin ligase site, non-target cell-specific transcriptional repression site, or non-target cell-specific epigenetic repression site. In some embodiments, the NTCSRE comprises a tissue-specific miRNA recognition sequence, tissue-specific protease recognition site, tissue-specific ubiquitin ligase site, tissue-specific transcriptional repression site, or tissue-specific epigenetic repression site. In some embodiments, the NTCSRE comprises a non-target cell-specific miRNA recognition sequence, non-target cell-specific protease recognition site, non-target cell-specific ubiquitin ligase site, non-target cell-specific transcriptional repression site, or non-target cell-specific epigenetic repression site. In some embodiments, the NTCSRE comprises a non-target cell-specific miRNA recognition sequence and the miRNA recognition sequence is able to be bound by one or more of miR3 1, miR363, or miR29c. In some embodiments, the NTCSRE is situated or encoded within a transcribed region encoding the exogenous agent, optionally wherein an RNA produced by the transcribed region comprises the miRNA recognition sequence within a UTR or coding region.

### 1. Nucleic Acids

In some embodiments, the exogenous agent may include a nucleic acid. For example, the exogenous agent may comprise RNA to enhance expression of an endogenous protein, or a siRNA or miRNA that inhibits protein expression of an endogenous protein. For example, the endogenous protein may modulate structure or function in the target cells. In some embodiments, the exogenous agent may include a nucleic acid encoding an engineered protein that modulates structure or function in the target cells. In some embodiments, the exogenous agent is a nucleic acid that targets a transcriptional activator that modulate structure or function in the target cells

In some embodiments, a lipid particle described herein comprises a nucleic acid, e.g., RNA or DNA. In some embodiments, the nucleic acid is, comprises, or consists of one or more natural nucleic acid residues. In some embodiments, the nucleic acid is, comprises, or consists of one or more nucleic acid analogs. In some embodiments, the nucleic acid has a nucleotide sequence that encodes a functional gene product such as an RNA or protein. In some embodiments, the nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, the nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, the nucleic acid is partly or wholly single stranded; in some embodiments, the nucleic acid is partly or wholly double stranded. In some embodiments the nucleic acid has a nucleotide sequence comprising at least one element that encodes, or is the complement of a sequence that encodes, a polypeptide. The nucleic acid may include variants, e.g., having an overall sequence identity with a reference nucleic acid of at least 85%, 86%, 87%, 88%, 89%, 90%, 91%, 92%, 93%, 94%, 95%, 96%, 97%, or 99%. In some embodiments, a variant nucleic acid does not share at least one characteristic sequence element with a reference nucleic acid. In some embodiments, a variant nucleic acid shares one or more of the biological activities of the reference nucleic acid. In some embodiments, a nucleic acid variant has a nucleic acid sequence that is identical to that of the reference but for a small number of sequence alterations at particular positions. In some embodiments, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant nucleic acid comprises about 10, about 9, about 8, about 7, about 6, about 5, about 4, about 3, about 2, or about 1 substituted residue as compared to a reference. In some embodiments, a variant nucleic acid comprises a very small number (e.g., fewer than about 5, about 4, about 3, about 2, or about 1) number of substituted, inserted, or deleted, functional residues that participate in a particular biological activity relative to the reference. In some embodiments, a variant nucleic acid comprises not more than about 15, about 12, about 9, about 3, or about 1 addition or deletion, and, in some embodiments, comprises no additions or deletions, as compared to the reference. In some embodiments, a variant nucleic acid comprises fewer than about 27, about 24, about 21, about 18, about 15, about 12, about 9, about 6, about 3, or fewer than about 9, about 6, about 3, or about 2 additions or deletions as compared to the reference.

In some embodiments, the exogenous agent includes a nucleic acid, e.g., DNA, nDNA (nuclear DNA), mtDNA (mitochondrial DNA), protein coding DNA, gene, operon, chromosome, genome, transposon, retrotransposon, viral genome, intron, exon, modified DNA, mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), lncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprograming RNAs, aptamers, and any combination thereof. In some embodiments, the nucleic acid is a wild-type nucleic acid. In some embodiments, the protein is a mutant nucleic acid. In some embodiments the nucleic acid is a fusion or chimera of multiple nucleic acid sequences

In embodiments, the nucleic acid encodes one or more (e.g. two or more) inhibitory RNA molecules directed against one or more RNA targets. An inhibitory RNA molecule can be, e.g., a miRNA or an shRNA. In some embodiments, the inhibitory molecule can be a precursor of a miRNA, such as for example, a Pri-miRNA or a Pre-miRNA, or a precursor of an shRNA. In some embodiments, the inhibitory molecule can be an artificially derived miRNA or shRNA. In other embodiments, the inhibitory RNA molecule can be a dsRNA (either transcribed or artificially introduced) that is processed into an siRNA or the siRNA itself. In some embodiments, the inhibitory RNA molecule can be a miRNA or shRNA that has a sequence that is not found in nature, or has at least one functional segment that is not found in nature, or has a combination of functional segments that are not found in nature. In illustrative embodiments, at least one or all of the inhibitory RNA molecules are miR-155. In some embodiments, a retroviral vector described herein encodes two or more inhibitory RNA molecules directed against one or more RNA targets. Two or more inhibitory RNA molecules, in some embodiments, can be directed against different targets. In other embodiments, the two or more inhibitory RNA molecules are directed against the same target. In some embodiments, the exogenous agent comprises a shRNA. A shRNA (short hairpin RNA) can comprise a double-stranded structure that is formed by a single self complementary RNA strand. shRNA constructs can comprise a nucleotide sequence identical to a portion, of either coding or non-coding sequence, of a target gene. RNA sequences with insertions, deletions, and single point mutations relative to the target sequence can also be used. Greater than 90% sequence identity, or even 100% sequence identity, between the inhibitory RNA and the portion of the target gene can be used. In certain embodiments, the length of the duplex-forming portion of an shRNA is at least 20, 21, or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size. In embodiments, a retroviral vector that encodes an siRNA, an miRNA, an shRNA, or a ribozyme comprises one or more regulatory sequences, such as, for example, a strong constitutive pol III, e.g., human U6 snRNA promoter, the mouse U6 snRNA promoter, the human and mouse H1RNA promoter and the human tRNA-val promoter, or a strong constitutive pol II promoter.

### 2. Polypeptides

In some embodiments, the lipid particle contains a nucleic acid that encodes a protein exogenous agent (also referred to as a “payload gene encoding an exogenous agent.”). In some embodiments, a lipid particle described herein comprises an exogenous agent which is or comprises a protein.

In some embodiments, the protein may include moieties other than amino acids (e.g., may be glycoproteins, proteoglycans, etc.) and/or may be otherwise processed or modified. In some embodiments, the protein can sometimes include more than one polypeptide chain, for example linked by one or more disulfide bonds or associated by other means.

In some embodiments, the protein may contain L-amino acids, D-amino acids, or both and may contain any of a variety of amino acid modifications or analogs. In some embodiments, proteins may comprise natural amino acids, non-natural amino acids, synthetic amino acids, and combinations thereof. In some embodiments, proteins are antibodies, antibody fragments, biologically active portions thereof, and/or characteristic portions thereof. In some embodiments, a polypeptide may include its variants, e.g., having an overall sequence identity with a reference polypeptide of at least 85%, 86%, 87%, 88%, 89%, 90%, 91%, 92%, 93%, 94%, 95%, 96%, 97%, or 99%. In some embodiments, a variant polypeptide does not share at least one characteristic sequence element with a reference polypeptide. In some embodiments, a variant polypeptide shares one or more of the biological activities of the reference polypeptide. In some embodiments, a polypeptide variant has an amino acid sequence that is identical to that of the reference but for a small number of sequence alterations at particular positions. In some embodiments, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide comprises about 10, about 9, about 8, about 7, about 6, about 5, about 4, about 3, about 2, or about 1 substituted residue as compared to a reference. In some embodiments, a variant polypeptide comprises a very small number (e.g., fewer than about 5, about 4, about 3, about 2, or about 1) number of substituted, inserted, or deleted, functional that participate in a particular biological activity relative to the reference. In some embodiments, a variant polypeptide comprises not more than about 5, about 4, about 3, about 2, or about 1 addition or deletion, and, in some embodiments, comprises no additions or deletions, as compared to the reference. In some embodiments, a variant polypeptide comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, the protein includes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), cas9 and homologs thereof), recombinases, and any combination thereof. In some embodiments, the protein targets a protein in the cell for degradation. In some embodiments, the protein targets a protein in the cell for degradation by localizing the protein to the proteasome. In some embodiments, the protein is a wild-type protein. In some embodiments, the protein is a mutant protein.

Exemplary protein exogenous agents are described in the following subsections. In some embodiments, a lipid particle provided herein can include any of such exogenous agents. In particular embodiments, a lipid particle contains a nucleic acid encoding any of such exogenous agents.

### A) Cytosolic Proteins

In some embodiments, the exogenous agent comprises a cytosolic protein, e.g., a protein that is produced in the recipient cell and localizes to the recipient cell cytoplasm. In some embodiments, the exogenous agent comprises a secreted protein, e.g., a protein that is produced and secreted by the recipient cell. In some embodiments, the exogenous agent comprises a nuclear protein, e.g., a protein that is produced in the recipient cell and is imported to the nucleus of the recipient cell. In some embodiments, the exogenous agent comprises an organellar protein (e.g., a mitochondrial protein), e.g., a protein that is produced in the recipient cell and is imported into an organelle (e.g., a mitochondrial) of the recipient cell. In some embodiments, the protein is a wild-type protein or a mutant protein. In some embodiments the protein is a fusion or chimeric protein.

### B) Membrane Proteins

In some embodiments, the exogenous agent comprises a membrane protein. In some embodiments, the membrane protein comprises a chimeric antigen receptor (CAR), a T cell receptor, an integrin, an ion channel, a pore forming protein, a Toll-Like Receptor, an interleukin receptor, a cell adhesion protein, or a transport protein.

### I. Chimeric Antigen Receptors (CARs)

In certain embodiments, the payload gene may comprise an exogenous polynucleotide encoding a CAR. CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. The polycistronic vector of the present disclosure may be used to express one or more CARs in a host cell (e.g., a T cell) for use in cell-based therapies against various target antigens. The CARs expressed by the one or more expression cassettes may be the same or different. In these embodiments, the CAR may comprise an extracellular binding domain (also referred to as a “binder”) that specifically binds a target antigen, a transmembrane domain, and an intracellular signaling domain. In certain embodiments, the CAR may further comprise one or more additional elements, including one or more signal peptides, one or more extracellular hinge domains, and/or one or more intracellular costimulatory domains. Domains may be directly adjacent to one another, or there may be one or more amino acids linking the domains. The nucleotide sequence encoding a CAR may be derived from a mammalian sequence, for example, a mouse sequence, a primate sequence, a human sequence, or combinations thereof. In the cases where the nucleotide sequence encoding a CAR is non-human, the sequence of the CAR may be humanized. The nucleotide sequence encoding a CAR may also be codon-optimized for expression in a mammalian cell, for example, a human cell. In any of these embodiments, the nucleotide sequence encoding a CAR may be at least 80% identical (e.g., at least 80%, at least 85%, at least 90%, at least 95%, at least 96%, at least 97%, at least 98%, at least 99%, or 100% identical) to any of the nucleotide sequences disclosed herein. The sequence variations may be due to codon-optimization, humanization, restriction enzyme-based cloning scars, and/or additional amino acid residues linking the functional domains, etc.

In certain embodiments, the CAR may comprise a signal peptide at the N-terminus. Non-limiting examples of signal peptides include CD8a signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 5 below.

TABLE 5
Exemplary sequences of signal peptides

| SEQ ID | | |
|---|---|---|
| NO: | Sequence | Description |
| 378 | MALPVTALLLPLALLLHAARP | CD8α signal |
| | | peptide |
| 379 | METDTLLLWVLLLWVPGSTG | IgK signal |
| | | peptide |
| 380 | MLLLVTSLLLCELPHPAFLLIP | GMCSFR-α |
| | | (CSF2RA) |
| | | signal |
| | | peptide |

In certain embodiments, the extracellular binding domain of the CAR may comprise one or more antibodies specific to one target antigen or multiple target antigens. The antibody may be an antibody fragment, for example, an scFv, or a single-domain antibody fragment, for example, a VHH. In certain embodiments, the scFv may comprise a heavy chain variable region (V_{H}) and a light chain variable region (V_{L}) of an antibody connected by a linker. The V_{H} and the V_{L} may be connected in either order, i.e., V_{H}-linker-V_{L} or V_{L}-linker-V_{H}. Non-limiting examples of linkers include Whitlow linker, (G₄S)ₙ (n can be a positive integer, e.g., 1, 2, 3, 4, 5, 6, etc.) linker, and variants thereof. In certain embodiments, the antigen may be an antigen that is exclusively or preferentially expressed on tumor cells, or an antigen that is characteristic of an autoimmune or inflammatory disease. Exemplary target antigens include, but are not limited to, CD5, CD19, CD20, CD22, CD23, CD30, CD70, Kappa, Lambda, and B cell maturation agent (BCMA), G-protein coupled receptor family C group 5 member D (GPRC5D) (associated with leukemias); CS1/SLAMF7, CD38, CD138, GPRC5D, TACI, and BCMA (associated with myelomas); GD2, HER2, EGFR, EGFRvIII, B7H3, PSMA, PSCA, CAIX, CD171, CEA, CSPG4, EPHA2, FAP, FRα, IL-13Rα, Mesothelin, MUC1, MUC16, and ROR1 (associated with solid tumors). In any of these embodiments, the extracellular binding domain of the CAR can be codon-optimized for expression in a host cell or have variant sequences to increase functions of the extracellular binding domain.

In certain embodiments, the CAR may comprise a hinge domain, also referred to as a spacer. The terms “hinge” and “spacer” may be used interchangeably in the present disclosure. Non-limiting examples of hinge domains include CD8α hinge domain, CD28 hinge domain, IgG4 hinge domain, IgG4 hinge-CH2-CH3 domain, and variants thereof, the amino acid sequences of which are provided in Table 6 below.

TABLE 6
Exemplary sequences of hinge domains

| SEQ ID | | |
|---|---|---|
| NO: | Sequence | Description |
| 381 | TTTPAPRPPTPAPTIAS | CD8α hinge domain |
| | QPLSLRPEACRPAAGGA | |
| | VHTRGLDFACD | |
| 382 | IEVMYPPPYLDNEKSNG | CD28 hinge domain |
| | TIIHVKGKHLCPSPLFP | |
| | GPSKP | |
| 383 | AAAIEVMYPPPYLDNEK | CD28 hinge domain |
| | SNGTIIHVKGKHLCPSP | |
| | LFPGPSKP | |
| 384 | ESKYGPPCPPCP | IgG4 hinge domain |
| 385 | ESKYGPPCPSCP | IgG4 hinge domain |
| 386 | ESKYGPPCPPCPAPEFL | IgG4 hinge-CH2-CH3 |
| | GGPSVFLFPPKPKDTLM | domain |
| | ISRTPEVTCVVVDVSQE | |
| | DPEVQFNWYVDGVEVHN | |
| | AKTKPREEQFNSTYRVV | |
| | SVLTVLHQDWLNGKEYK | |
| | CKVSNKGLPSSIEKTIS | |
| | KAKGQPREPQVYTLPPS | |
| | QEEMTKNQVSLTCLVKG | |
| | FYPSDIAVEWESNGQPE | |
| | NNYKTTPPVLDSDGSFF | |
| | LYSRLTVDKSRWQEGNV | |
| | FSCSVMHEALHNHYTQK | |
| | SLSLSLGK | |

In certain embodiments, the transmembrane domain of the CAR may comprise a transmembrane region of the alpha, beta, or zeta chain of a T cell receptor, CD28, CD3E, CD45, CD4, CD5, CD8, CD9, CD16, CD22, CD33, CD37, CD64, CD80, CD86, CD134, CD137, CD154, or a functional variant thereof, including the human versions of each of these sequences. In other embodiments, the transmembrane domain may comprise a transmembrane region of CD8α, CD80, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or a functional variant thereof, including the human versions of each of these sequences. Table 7 provides the amino acid sequences of a few exemplary transmembrane domains.

TABLE 7
Exemplary sequences of transmembrane domains

| SEQ ID | | |
|---|---|---|
| NO: | Sequence | Description |
| 387 | IYIWAPLAGTCGVLLL | CD8α transmembrane |
| | SLVITLYC | domain |
| 388 | FWVLVVVGGVLACYSL | CD28 transmembrane |
| | LVTVAFIIFWV | domain |
| 389 | MFWVLVVVGGVLACYS | CD28 transmembrane |
| | LLVTVAFIIFWV | domain |

In certain embodiments, the intracellular signaling domain and/or intracellular costimulatory domain of the CAR may comprise one or more signaling domains selected from B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD3ζ, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain and/or intracellular costimulatory domain comprises one or more signaling domains selected from a CD3ζ domain, an ITAM, a CD28 domain, 4-1BB domain, or a functional variant thereof. Table 8 provides the amino acid sequences of a few exemplary intracellular costimulatory and/or signaling domains. In certain embodiments, as in the case of tisagenlecleucel as described below, the CD3ζ signaling domain of SEQ ID NO:392 may have a mutation, e.g., a glutamine (Q) to lysine (K) mutation, at amino acid position 14 (see SEQ ID NO:393).

TABLE 8
Exemplary sequences of intracellular
costimulatory and/or signaling domains

| SEQ ID | | |
|---|---|---|
| NO: | Sequence | Description |
| 390 | KRGRKKLLYIFKQPFMRPVQTTQEEDGCSC | 4-1BB costimulatory |
| | RFPEEEEGGCEL | domain |
| 391 | RSKRSRLLHSDYMNMTPRRPGPTRKHYQPY | CD28 costimulatory |
| | APPRDFAAYRS | domain |
| 392 | RVKFSRSADAPAYQQGQNQLYNELNLGRRE | CD3ζ signaling domain |
| | EYDVLDKRRGRDPEMGGKPRRKNPQEGLYN | |
| | ELQKDKMAEAYSEIGMKGERRRGKGHDGLY | |
| | QGLSTATKDTYDALHMQALPPR | |
| 393 | RVKFSRSADAPAYKQGQNQLYNELNLGRRE | CD3ζ signaling domain |
| | EYDVLDKRRGRDPEMGGKPRRKNPQEGLYN | (with Q to K mutation |
| | ELQKDKMAEAYSEIGMKGERRRGKGHDGLY | at position 14) |
| | QGLSTATKDTYDALHMQALPPR | |

In certain embodiments where the polycistronic vector encodes two or more CARs, the two or more CARs may comprise the same functional domains, or one or more different functional domains, as described. For example, the two or more CARs may comprise different signal peptides, extracellular binding domains, hinge domains, transmembrane domains, costimulatory domains, and/or intracellular signaling domains, in order to minimize the risk of recombination due to sequence similarities. Or, alternatively, the two or more CARs may comprise the same domains. In the cases where the same domain(s) and/or backbone are used, it is optional to introduce codon divergence at the nucleotide sequence level to minimize the risk of recombination.

### a) CD19 CAR

In some embodiments, the CAR is a CD19 CAR (“CD19-CAR”), and in these embodiments, the polycistronic vector comprises an expression cassette that contains a nucleotide sequence encoding a CD19 CAR. In some embodiments, the CD19 CAR may comprise a signal peptide, an extracellular binding domain that specifically binds CD19, a hinge domain, a transmembrane domain, an intracellular costimulatory domain, and/or an intracellular signaling domain in tandem.

In some embodiments, the signal peptide of the CD19 CAR comprises a CD8α signal peptide. In some embodiments, the CD8α signal peptide comprises or consists of an amino acid sequence set forth in SEQ ID NO:378 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 378. In some embodiments, the signal peptide comprises an IgK signal peptide. In some embodiments, the IgK signal peptide comprises or consists of an amino acid sequence set forth in SEQ ID NO:379 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 379. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:380 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 380.

In some embodiments, the extracellular binding domain of the CD19 CAR is specific to CD19, for example, human CD19. The extracellular binding domain of the CD19 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD19 CAR comprises an scFv derived from the FMC63 monoclonal antibody (FMC63), which comprises the heavy chain variable region (V_{H}) and the light chain variable region (V_{L}) of FMC63 connected by a linker. FMC63 and the derived scFv have been described in Nicholson et al., Mol. Immun. 34(16-17):1157-1165 (1997) and PCT Application Publication No. WO2018/213337, the entire contents of each of which are incorporated by reference herein. In some embodiments, the amino acid sequences of the entire FMC63-derived scFv (also referred to as FMC63 scFv) and its different portions are provided in Table 9 below. In some embodiments, the CD19-specific scFv comprises or consists of an amino acid sequence set forth in SEQ ID NO:394, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:394. In some embodiments, the CD19-specific scFv may comprise a light chain variable region that comprises or consists of an amino acid sequence set forth in SEQ ID NO: 395, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:395. In some embodiments, the CD19-specific scFv may comprise a heavy chain variable region that comprises or consists of an amino acid sequence set forth in SEQ ID NO: 400, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:400. In some embodiments, the CD19-specific scFv may comprise one or more CDRs having amino acid sequences set forth in SEQ ID NOs: 396-398 and 401-403. In some embodiments, the CD19-specific scFv may comprise a light chain with one or more CDRs having amino acid sequences set forth in SEQ ID NOs: 396-398. In some embodiments, the CD19-specific scFv may comprise a heavy chain with one or more CDRs having amino acid sequences set forth in SEQ ID NOs: 401-403. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the linker linking the V_{H} and the V_{L} portions of the scFv is a Whitlow linker having an amino acid sequence set forth in SEQ ID NO:399. In some embodiments, the Whitlow linker may be replaced by a different linker, for example, a 3xG₄S linker having an amino acid sequence set forth in SEQ ID NO:405, which gives rise to a different FMC63-derived scFv having an amino acid sequence set forth in SEQ ID NO:404. In certain of these embodiments, the CD19-specific scFv comprises or consists of an amino acid sequence set forth in SEQ ID NO:404 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:404.

TABLE 9
Exemplary sequences of anti-CD19 scFv and components

| SEQ ID NO: | Amino Acid Sequence | Description |
|---|---|---|
| 394 | DIQMTQTTSSLSASLGDRVTISCRASQDI | Anti-CD19 FMC63 scFv |
| | SKYLNWYQQKPDGTVKLLIYHTSRLHS | entire sequence, with |
| | GVPSRFSGSGSGTDYSLTISNLEQEDIAT | Whitlow linker |
| | YFCQQGNTLPYTFGGGTKLEITGSTSGS | |
| | GKPGSGEGSTKGEVKLQESGPGLVAPS | |
| | QSLSVTCTVSGVSLPDYGVSWIRQPPRK | |
| | GLEWLGVIWGSETTYYNSALKSRLTIIK | |
| | DNSKSQVFLKMNSLQTDDTAIYYCAKH | |
| | YYYGGSYAMDYWGQGTSVTVSS | |
| 395 | DIQMTQTTSSLSASLGDRVTISCRASQDI | Anti-CD19 FMC63 scFv |
| | SKYLNWYQQKPDGTVKLLIYHTSRLHS | light chain variable |
| | GVPSRFSGSGSGTDYSLTISNLEQEDIAT | region |
| | YFCQQGNTLPYTFGGGTKLEIT | |
| 396 | QDISKY | Anti-CD19 FMC63 scFv |
| | | light chain CDR1 |
| 397 | HTS | Anti-CD19 FMC63 scFv |
| | | light chain CDR2 |
| 398 | QQGNTLPYT | Anti-CD19 FMC63 scFv |
| | | light chain CDR3 |
| 399 | GSTSGSGKPGSGEGSTKG | Whitlow linker |
| 400 | EVKLQESGPGLVAPSQSLSVTCTVSGVS | Anti-CD19 FMC63 scFv |
| | LPDYGVSWIRQPPRKGLEWLGVIWGSE | heavy chain variable |
| | TTYYNSALKSRLTIIKDNSKSQVFLKMN | region |
| | SLQTDDTAIYYCAKHYYYGGSYAMDY | |
| | WGQGTSVTVSS | |
| 401 | GVSLPDYG | Anti-CD19 FMC63 scFv |
| | | heavy chain CDR1 |
| 402 | IWGSETT | Anti-CD19 FMC63 scFv |
| | | heavy chain CDR2 |
| 403 | AKHYYYGGSYAMDY | Anti-CD19 FMC63 scFv |
| | | heavy chain CDR3 |
| 404 | DIQMTQTTSSLSASLGDRVTISCRASQDI | Anti-CD19 FMC63 scFv |
| | SKYLNWYQQKPDGTVKLLIYHTSRLHS | entire sequence, with |
| | GVPSRFSGSGSGTDYSLTISNLEQEDIAT | 3xG₄S linker |
| | YFCQQGNTLPYTFGGGTKLEITGGGGS | |
| | GGGGSGGGGSEVKLQESGPGLVAPSQS | |
| | LSVTCTVSGVSLPDYGVSWIRQPPRKGL | |
| | EWLGVIWGSETTYYNSALKSRLTIIKDN | |
| | SKSQVFLKMNSLQTDDTAIYYCAKHYY | |
| | YGGSYAMDYWGQGTSVTVSS | |
| 405 | GGGGSGGGGSGGGGS | 3xG₄S linker |

In some embodiments, the extracellular binding domain of the CD19 CAR is derived from an antibody specific to CD19, including, for example, SJ25C1 (Bejcek et al., Cancer Res. 55:2346-2351 (1995)), HD37 (Pezutto et al., J. Immunol. 138(9):2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Callard et al., J. Immunology, 148(10): 2983-2987 (1992)), and CLB-CD19 (De Rie Cell. Immunol. 118:368-381(1989)). In any of these embodiments, the extracellular binding domain of the CD19 CAR can comprise or consist of the V_{H}, the V_{L}, and/or one or more CDRs of any of the antibodies.

In some embodiments, the hinge domain of the CD19 CAR comprises a CD8α hinge domain, for example, a human CD8α hinge domain. In some embodiments, the CD8α hinge domain comprises or consists of an amino acid sequence set forth in SEQ ID NO:381 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:381. In some embodiments, the hinge domain comprises a CD28 hinge domain, for example, a human CD28 hinge domain. In some embodiments, the CD28 hinge domain comprises or consists of an amino acid sequence set forth in SEQ ID NO:382 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:382. In some embodiments, the CD28 hinge domain comprises or consists of an amino acid sequence set forth in SEQ ID NO:383 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:383. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:384 or SEQ ID NO:385, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:384 or SEQ ID NO:385. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:386 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:386.

In some embodiments, the transmembrane domain of the CD19 CAR comprises a CD8α transmembrane domain, for example, a human CD8α transmembrane domain. In some embodiments, the CD8α transmembrane domain comprises or consists of an amino acid sequence set forth in SEQ ID NO:387 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:387. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:388 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:388. In some embodiments, the CD28 transmembrane domain comprises or consists of an amino acid sequence set forth in SEQ ID NO:389 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:389.

In some embodiments, the intracellular costimulatory domain of the CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the 4-1BB costimulatory domain comprises or consists of an amino acid sequence set forth in SEQ ID NO:390 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:390. In some embodiments, the intracellular costimulatory domain comprises a CD28 costimulatory domain. CD28 is another co-stimulatory molecule on T cells. In some embodiments, the CD28 costimulatory domain is human. In some embodiments, the CD28 costimulatory domain comprises or consists of an amino acid sequence set forth in SEQ ID NO:391 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:391. In some embodiments, the intracellular costimulatory domain of the CD19 CAR comprises a 4-1BB costimulatory domain and a CD28 costimulatory domain as described.

In some embodiments, the intracellular signaling domain of the CD19 CAR comprises a CD3 zeta (ζ) signaling domain. CD3ζ associates with T cell receptors (TCRs) to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In some embodiments, the CD3ζ signaling domain is human. In some embodiments, the CD3ζ signaling domain comprises or consists of an amino acid sequence set forth in SEQ ID NO:392 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:392.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:394 or SEQ ID NO:404, the CD8α hinge domain of SEQ ID NO:381, the CD8α transmembrane domain of SEQ ID NO:387, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:394 or SEQ ID NO:404, the IgG4 hinge domain of SEQ ID NO:384 or SEQ ID NO:385, the CD28 transmembrane domain of SEQ ID NO:388, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:394 or SEQ ID NO:404, the CD28 hinge domain of SEQ ID NO:383, the CD28 transmembrane domain of SEQ ID NO:388, the CD28 costimulatory domain of SEQ ID NO:391, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR as set forth in SEQ ID NO:406 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:406 (see Table 10). The encoded CD19 CAR has a corresponding amino acid sequence set forth in SEQ ID NO:407 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:407, with the following components: CD8α signal peptide, FMC63 scFv (V_{L}-Whitlow linker-V_{H}), CD8α hinge domain, CD8α transmembrane domain, 4-1BB costimulatory domain, and CD3ζ signaling domain.

In some embodiments, the polycistronic vector comprises an expression cassette that contains a nucleotide sequence encoding a commercially available embodiment of CD19 CAR. Non-limiting examples of commercially available embodiments of CD19 CARs expressed and/or encoded by T cells include tisagenlecleucel, lisocabtagene maraleucel, axicabtagene ciloleucel, and brexucabtagene autoleucel.

In some embodiments, the polycistronic vector comprises an expression cassette that contains a nucleotide sequence encoding tisagenlecleucel or portions thereof. Tisagenlecleucel comprises a CD19 CAR with the following components: CD8α signal peptide, FMC63 scFv (V_{L}-3ₓG₄S linker-V_{H}), CD8α hinge domain, CD8α transmembrane domain, 4-1BB costimulatory domain, and CD3ζ signaling domain. The nucleotide and amino acid sequence of the CD19 CAR in tisagenlecleucel are provided in Table 10, with annotations of the sequences provided in Table 11.

In some embodiments, the polycistronic vector comprises an expression cassette that contains a nucleotide sequence encoding lisocabtagene maraleucel or portions thereof. Lisocabtagene maraleucel comprises a CD19 CAR with the following components: GMCSFR-α or CSF2RA signal peptide, FMC63 scFv (V_{L}-Whitlow linker-V_{H}), IgG4 hinge domain, CD28 transmembrane domain, 4-1BB costimulatory domain, and CD3ζ signaling domain. The nucleotide and amino acid sequence of the CD19 CAR in lisocabtagene maraleucel are provided in Table 10, with annotations of the sequences provided in Table 12.

In some embodiments, the polycistronic vector comprises an expression cassette that contains a nucleotide sequence encoding axicabtagene ciloleucel or portions thereof. Axicabtagene ciloleucel comprises a CD19 CAR with the following components: GMCSFR-α or CSF2RA signal peptide, FMC63 scFv (V_{L}-Whitlow linker-V_{H}), CD28 hinge domain, CD28 transmembrane domain, CD28 costimulatory domain, and CD3ζ signaling domain. The nucleotide and amino acid sequence of the CD19 CAR in axicabtagene ciloleucel are provided in Table 10, with annotations of the sequences provided in Table 13.

In some embodiments, the polycistronic vector comprises an expression cassette that contains a nucleotide sequence encoding brexucabtagene autoleucel or portions thereof. Brexucabtagene autoleucel comprises a CD19 CAR with the following components: GMCSFR-α signal peptide, FMC63 scFv, CD28 hinge domain, CD28 transmembrane domain, CD28 costimulatory domain, and CD3ζ signaling domain.

In some embodiments, the polycistronic vector comprises an expression cassette that contains a nucleotide sequence encoding a CD19 CAR as set forth in SEQ ID NO: 408, 410, or 412, or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 408, 410, or 412. The encoded CD19 CAR has a corresponding amino acid sequence set forth in SEQ ID NO: 409, 411, or 413, respectively, or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 409, 411, or 413, respectively.

TABLE 10
Exemplary sequences of CD19 CARs

| SEQ ID NO: | Sequence | Description |
|---|---|---|
| 406 | atggccttaccagtgaccgccttgctcctgccgctggccttgctgctccac | Exemplary CD19 |
| | gccgccaggccggacatccagatgacacagactacatcctccctgtctgc | CAR nucleotide |
| | ctctctgggagacagagtcaccatcagttgcagggcaagtcaggacatta | sequence |
| | gtaaatatttaaattggtatcagcagaaaccagatggaactgttaaactcct | |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggaccaagctggagatcacaggctccacctctggatccggca | |
| | agcccggatctggcgagggatccaccaagggcgaggtgaaactgcag | |
| | gagtcaggacctggcctggtggcgccctcacagagcctgtccgtcacat | |
| | gcactgtctcaggggtctcattacccgactatggtgtaagctggattcgcc | |
| | agcctccacgaaagggtctggagtggctgggagtaatatggggtagtga | |
| | aaccacatactataattcagctctcaaatccagactgaccatcatcaagga | |
| | caactccaagagccaagttttcttaaaaatgaacagtctgcaaactgatga | |
| | cacagccatttactactgtgccaaacattattactacggtggtagctatgcta | |
| | tggactactggggccaaggaacctcagtcaccgtctcctcaaccacgac | |
| | gccagcgccgcgaccaccaacaccggcgcccaccatcgcgtcgcagc | |
| | ccctgtccctgcgcccagaggcgtgccggccagcggcggggggcgca | |
| | gtgcacacgagggggctggacttcgcctgtgatatctacatctgggcgcc | |
| | cttggccgggacttgtggggtccttctcctgtcactggttatcaccctttact | |
| | gcaaacggggcagaaagaaactcctgtatatattcaaacaaccatttatga | |
| | gaccagtacaaactactcaagaggaagatggctgtagctgccgatttcca | |
| | gaagaagaagaaggaggatgtgaactgagagtgaagttcagcaggagc | |
| | gcagacgcccccgcgtaccagcagggccagaaccagctctataacgag | |
| | ctcaatctaggacgaagagaggagtacgatgttttggacaagagacgtgg | |
| | ccgggaccctgagatggggggaaagccgagaaggaagaaccctcagg | |
| | aaggcctgtacaatgaactgcagaaagataagatggcggaggcctacag | |
| | tgagattgggatgaaaggcgagcgccggaggggcaaggggcacgatg | |
| | gcctttaccagggtctcagtacagccaccaaggacacctacgacgccctt | |
| | cacatgcaggccctgccccctcgc | |
| 407 | MALPVTALLLPLALLLHAARPDIQMTQTTSSLS | Exemplary CD19 |
| | ASLGDRVTISCRASQDISKYLNWYQQKPDGTV | CAR amino acid |
| | KLLIYHTSRLHSGVPSRFSGSGSGTDYSLTISNL | sequence |
| | EQEDIATYFCQQGNTLPYTFGGGTKLEITGSTS | |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSTTTPAPRPPTPAPTIASQPLSLRPEACRP | |
| | AAGGAVHTRGLDFACDIYIWAPLAGTCGVLLL | |
| | SLVITLYCKRGRKKLLYIFKQPFMRPVQTTQEE | |
| | DGCSCRFPEEEEGGCELRVKFSRSADAPAYQQ | |
| | GQNQLYNELNLGRREEYDVLDKRRGRDPEMG | |
| | GKPRRKNPQEGLYNELQKDKMAEAYSEIGMK | |
| | GERRRGKGHDGLYQGLSTATKDTYDALHMQA | |
| | LPPR | |
| 408 | atggccttaccagtgaccgccttgctcctgccgctggccttgctgctccac | Tisagenlecleucel |
| | gccgccaggccggacatccagatgacacagactacatcctccctgtctgc | CD19 CAR |
| | ctctctgggagacagagtcaccatcagttgcagggcaagtcaggacatta | nucleotide sequence |
| | gtaaatatttaaattggtatcagcagaaaccagatggaactgttaaactcct | |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggaccaagctggagatcacaggtggcggtggctcgggcggt | |
| | ggtgggtcgggtggcggcggatctgaggtgaaactgcaggagtcagga | |
| | cctggcctggtggcgccctcacagagcctgtccgtcacatgcactgtctc | |
| | aggggtctcattacccgactatggtgtaagctggattcgccagcctccacg | |
| | aaagggtctggagtggctgggagtaatatggggtagtgaaaccacatact | |
| | ataattcagctctcaaatccagactgaccatcatcaaggacaactccaaga | |
| | gccaagttttcttaaaaatgaacagtctgcaaactgatgacacagccattta | |
| | ctactgtgccaaacattattactacggtggtagctatgctatggactactgg | |
| | ggccaaggaacctcagtcaccgtctcctcaaccacgacgccagcgccg | |
| | cgaccaccaacaccggcgcccaccatcgcgtcgcagcccctgtccctgc | |
| | gcccagaggcgtgccggccagcggcggggggcgcagtgcacacgag | |
| | ggggctggacttcgcctgtgatatctacatctgggcgcccttggccggga | |
| | cttgtggggtccttctcctgtcactggttatcaccctttactgcaaacggggc | |
| | agaaagaaactcctgtatatattcaaacaaccatttatgagaccagtacaaa | |
| | ctactcaagaggaagatggctgtagctgccgatttccagaagaagaagaa | |
| | ggaggatgtgaactgagagtgaagttcagcaggagcgcagacgccccc | |
| | gcgtacaagcagggccagaaccagctctataacgagctcaatctaggac | |
| | gaagagaggagtacgatgttttggacaagagacgtggccgggaccctga | |
| | gatggggggaaagccgagaaggaagaaccctcaggaaggcctgtaca | |
| | atgaactgcagaaagataagatggcggaggcctacagtgagattgggat | |
| | gaaaggcgagcgccggaggggcaaggggcacgatggcctttaccagg | |
| | gtctcagtacagccaccaaggacacctacgacgcccttcacatgcaggc | |
| | cctgccccctcgc | |
| 409 | MALPVTALLLPLALLLHAARPDIQMTQTTSSLS | Tisagenlecleucel |
| | ASLGDRVTISCRASQDISKYLNWYQQKPDGTV | CD19 CAR amino |
| | KLLIYHTSRLHSGVPSRFSGSGSGTDYSLTISNL | acid sequence |
| | EQEDIATYFCQQGNTLPYTFGGGTKLEITGGGG | |
| | SGGGGSGGGGSEVKLQESGPGLVAPSQSLSVT | |
| | CTVSGVSLPDYGVSWIRQPPRKGLEWLGVIWG | |
| | SETTYYNSALKSRLTIIKDNSKSQVFLKMNSLQ | |
| | TDDTAIYYCAKHYYYGGSYAMDYWGQGTSV | |
| | TVSSTTTPAPRPPTPAPTIASQPLSLRPEACRPA | |
| | AGGAVHTRGLDFACDIYIWAPLAGTCGVLLLS | |
| | LVITLYCKRGRKKLLYIFKQPFMRPVQTTQEED | |
| | GCSCRFPEEEEGGCELRVKFSRSADAPAYKQG | |
| | QNQLYNELNLGRREEYDVLDKRRGRDPEMGG | |
| | KPRRKNPQEGLYNELQKDKMAEAYSEIGMKG | |
| | ERRRGKGHDGLYQGLSTATKDTYDALHMQAL | |
| | PPR | |
| 410 | atgctgctgctggtgaccagcctgctgctgtgcgagctgccccaccccgc | Lisocabtagene |
| | ctttctgctgatccccgacatccagatgacccagaccacctccagcctgag | maraleucel CD19 |
| | cgccagcctgggcgaccgggtgaccatcagctgccgggccagccagg | CAR nucleotide |
| | acatcagcaagtacctgaactggtatcagcagaagcccgacggcaccgt | sequence |
| | caagctgctgatctaccacaccagccggctgcacagcggcgtgcccagc | |
| | cggtttagcggcagcggctccggcaccgactacagcctgaccatctcca | |
| | acctggaacaggaagatatcgccacctacttttgccagcagggcaacaca | |
| | ctgccctacacctttggcggcggaacaaagctggaaatcaccggcagca | |
| | cctccggcagcggcaagcctggcagcggcgagggcagcaccaaggg | |
| | cgaggtgaagctgcaggaaagcggccctggcctggtggcccccagcca | |
| | gagcctgagcgtgacctgcaccgtgagcggcgtgagcctgcccgacta | |
| | cggcgtgagctggatccggcagccccccaggaagggcctggaatggct | |
| | gggcgtgatctggggcagcgagaccacctactacaacagcgccctgaa | |
| | gagccggctgaccatcatcaaggacaacagcaagagccaggtgttcctg | |
| | aagatgaacagcctgcagaccgacgacaccgccatctactactgcgcca | |
| | agcactactactacggcggcagctacgccatggactactggggccaggg | |
| | caccagcgtgaccgtgagcagcgaatctaagtacggaccgccctgcccc | |
| | ccttgccctatgttctgggtgctggtggtggtcggaggcgtgctggcctgc | |
| | tacagcctgctggtcaccgtggccttcatcatcttttgggtgaaacggggc | |
| | agaaagaaactcctgtatatattcaaacaaccatttatgagaccagtacaaa | |
| | ctactcaagaggaagatggctgtagctgccgatttccagaagaagaagaa | |
| | ggaggatgtgaactgcgggtgaagttcagcagaagcgccgacgcccct | |
| | gcctaccagcagggccagaatcagctgtacaacgagctgaacctgggc | |
| | agaagggaagagtacgacgtcctggataagcggagaggccgggaccc | |
| | tgagatgggcggcaagcctcggcggaagaacccccaggaaggcctgta | |
| | taacgaactgcagaaagacaagatggccgaggcctacagcgagatcgg | |
| | catgaagggcgagcggaggcggggcaagggccacgacggcctgtatc | |
| | agggcctgtccaccgccaccaaggatacctacgacgccctgcacatgca | |
| | ggccctgcccccaagg | |
| 411 | MLLLVTSLLLCELPHPAFLLIPDIQMTQTTSSLS | Lisocabtagene |
| | ASLGDRVTISCRASQDISKYLNWYQQKPDGTV | maraleucel CD19 |
| | KLLIYHTSRLHSGVPSRFSGSGSGTDYSLTISNL | CAR amino acid |
| | EQEDIATYFCQQGNTLPYTFGGGTKLEITGSTS | sequence |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSESKYGPPCPPCPMFWVLVVVGGVLAC | |
| | YSLLVTVAFIIFWVKRGRKKLLYIFKQPFMRPV | |
| | QTTQEEDGCSCRFPEEEEGGCELRVKFSRSADA | |
| | PAYQQGQNQLYNELNLGRREEYDVLDKRRGR | |
| | DPEMGGKPRRKNPQEGLYNELQKDKMAEAYS | |
| | EIGMKGERRRGKGHDGLYQGLSTATKDTYDA | |
| | LHMQALPPR | |
| 412 | atgcttctcctggtgacaagccttctgctctgtgagttaccacacccagcatt | Axicabtagene |
| | cctcctgatcccagacatccagatgacacagactacatcctccctgtctgc | ciloleucel CD19 |
| | ctctctgggagacagagtcaccatcagttgcagggcaagtcaggacatta | CAR nucleotide |
| | gtaaatatttaaattggtatcagcagaaaccagatggaactgttaaactcct | sequence |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggactaagttggaaataacaggctccacctctggatccggcaa | |
| | gcccggatctggcgagggatccaccaagggcgaggtgaaactgcagg | |
| | agtcaggacctggcctggtggcgccctcacagagcctgtccgtcacatg | |
| | cactgtctcaggggtctcattacccgactatggtgtaagctggattcgcca | |
| | gcctccacgaaagggtctggagtggctgggagtaatatggggtagtgaa | |
| | accacatactataattcagctctcaaatccagactgaccatcatcaaggac | |
| | aactccaagagccaagttttcttaaaaatgaacagtctgcaaactgatgac | |
| | acagccatttactactgtgccaaacattattactacggtggtagctatgctat | |
| | ggactactggggtcaaggaacctcagtcaccgtctcctcagcggccgca | |
| | attgaagttatgtatcctcctccttacctagacaatgagaagagcaatggaa | |
| | ccattatccatgtgaaagggaaacacctttgtccaagtcccctatttcccgg | |
| | accttctaagcccttttgggtgctggtggtggttgggggagtcctggcttgc | |
| | tatagcttgctagtaacagtggcctttattattttctgggtgaggagtaagag | |
| | gagcaggctcctgcacagtgactacatgaacatgactccccgccgcccc | |
| | gggcccacccgcaagcattaccagccctatgccccaccacgcgacttcg | |
| | cagcctatcgctccagagtgaagttcagcaggagcgcagacgcccccg | |
| | cgtaccagcagggccagaaccagctctataacgagctcaatctaggacg | |
| | aagagaggagtacgatgttttggacaagagacgtggccgggaccctgag | |
| | atggggggaaagccgagaaggaagaaccctcaggaaggcctgtacaat | |
| | gaactgcagaaagataagatggcggaggcctacagtgagattgggatga | |
| | aaggcgagcgccggaggggcaaggggcacgatggcctttaccagggt | |
| | ctcagtacagccaccaaggacacctacgacgcccttcacatgcaggccc | |
| | tgccccctcgc | |
| 413 | MLLLVTSLLLCELPHPAFLLIPDIQMTQTTSSLS | Axicabtagene |
| | ASLGDRVTISCRASQDISKYLNWYQQKPDGTV | ciloleucel CD19 |
| | KLLIYHTSRLHSGVPSRFSGSGSGTDYSLTISNL | CAR amino acid |
| | EQEDIATYFCQQGNTLPYTFGGGTKLEITGSTS | sequence |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSAAAIEVMYPPPYLDNEKSNGTIIHVKG | |
| | KHLCPSPLFPGPSKPFWVLVVVGGVLACYSLL | |
| | VTVAFIIFWVRSKRSRLLHSDYMNMTPRRPGPT | |
| | RKHYQPYAPPRDFAAYRSRVKFSRSADAPAYQ | |
| | QGQNQLYNELNLGRREEYDVLDKRRGRDPEM | |
| | GGKPRRKNPQEGLYNELQKDKMAEAYSEIGM | |
| | KGERRRGKGHDGLYQGLSTATKDTYDALHMQ | |
| | ALPPR | |

TABLE 11
Annotation of tisagenlecleucel CD19 CAR sequences

| | Nucleotide Sequence | Amino Acid Sequence |
|---|---|---|
| Feature | Position | Position |
| CD8α signal peptide | 1-63 | 1-21 |
| FMC63 scFv | 64-789 | 22-263 |
| (V_{L}-3xG₄S linker-V_{H}) | | |
| CD8α hinge domain | 790-924 | 264-308 |
| CD8α transmembrane domain | 925-996 | 309-332 |
| 4-1BB costimulatory domain | 997-1122 | 333-374 |
| CD3ξ signaling domain | 1123-1458 | 375-486 |

TABLE 12
Annotation of lisocabtagene maraleucel CD19 CAR sequences

| | Nucleotide Sequence | Amino Acid Sequence |
|---|---|---|
| Feature | Position | Position |
| GMCSFR-a signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB costimulatory domain | 922-1047 | 308-349 |
| CD3ξ signaling domain | 1048-1383 | 350-461 |

TABLE 13
Annotation of axicabtagene ciloleucel CD19 CAR sequences

| | Nucleotide Sequence | Amino Acid Sequence |
|---|---|---|
| Feature | Position | Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 costimulatory domain | 1009-1131 | 337-377 |
| CD3ξ signaling domain | 1132-1467 | 378-489 |

In some embodiments, the polycistronic vector comprises an expression cassette that contains a nucleotide sequence encoding CD 19 CAR as set forth in SEQ ID NO: 408, 410, or 412, or at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 408, 410, or 412. The encoded CD19 CAR has a corresponding amino acid sequence set forth in SEQ ID NO: 409, 411, or 412, respectively, is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 409, 411, or 412, respectively.

### b) CD20 CAR

In some embodiments, the CAR is a CD20 CAR (“CD20-CAR”), and in these embodiments, the polycistronic vector comprises an expression cassette that contains a nucleotide sequence encoding a CD20 CAR. CD20 is an antigen found on the surface of B cells as early at the pro-B phase and progressively at increasing levels until B cell maturity, as well as on the cells of most B-cell neoplasms. CD20 positive cells are also sometimes found in cases of Hodgkin's disease, myeloma, and thymoma. In some embodiments, the CD20 CAR may comprise a signal peptide, an extracellular binding domain that specifically binds CD20, a hinge domain, a transmembrane domain, an intracellular costimulatory domain, and/or an intracellular signaling domain in tandem.

In some embodiments, the signal peptide of the CD20 CAR comprises a CD8α signal peptide. In some embodiments, the CD8α signal peptide comprises or consists of an amino acid sequence set forth in SEQ ID NO:378 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:378. In some embodiments, the signal peptide comprises an IgK signal peptide. In some embodiments, the IgK signal peptide comprises or consists of an amino acid sequence set forth in SEQ ID NO:379 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:379. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:380 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:380.

In some embodiments, the extracellular binding domain of the CD20 CAR is specific to CD20, for example, human CD20. The extracellular binding domain of the CD20 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD20 CAR is derived from an antibody specific to CD20, including, for example, Len16, IF5, 1.5.3, rituximab, obinutuzumab, ibritumomab, ofatumumab, tositumumab, odronextamab, veltuzumab, ublituximab, and ocrelizumab. In any of these embodiments, the extracellular binding domain of the CD20 CAR can comprise or consist of the V_{H}, the V_{L}, and/or one or more CDRs of any of the antibodies.

In some embodiments, the extracellular binding domain of the CD20 CAR comprises an scFv derived from the Leu16 monoclonal antibody, which comprises the heavy chain variable region (V_{H}) and the light chain variable region (V_{L}) of Leu16 connected by a linker. See Wu et al., Protein Engineering. 14(12):1025-1033 (2001). In some embodiments, the linker is a 3xG₄S linker. In other embodiments, the linker is a Whitlow linker as described herein. In some embodiments, the amino acid sequences of different portions of the entire Leu16-derived scFv (also referred to as Leu16 scFv) and its different portions are provided in Table 14 below. In some embodiments, the CD20-specific scFv comprises or consists of an amino acid sequence set forth in SEQ ID NO:414, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:414. In some embodiments, the CD20-specific scFv comprises or consists of a light chain variable region comprising or consisting of an amino acid sequence set forth in SEQ ID NO:415, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:415. In some embodiments, the CD20-specific scFv comprises or consists of a heavy chain variable region comprising or consisting of an amino acid sequence set forth in SEQ ID NO:419, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:419. In some embodiments, the CD20-specific scFv may comprise one or more CDRs having amino acid sequences set forth in SEQ ID NOs: 416-418, 420-422. In some embodiments, the CD20-specific scFv may comprise a light chain with one or more CDRs having amino acid sequences set forth in SEQ ID NOs: 416-418. In some embodiments, the CD20-specific scFv may comprise a heavy chain with one or more CDRs having amino acid sequences set forth in SEQ ID NOs: 420-422. In any of these embodiments, the CD20-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD20 CAR comprises or consists of the one or more CDRs as described herein.

TABLE 14
Exemplary sequences of anti-CD20 scFv and components

| SEQ ID NO: | Amino Acid Sequence | Description |
|---|---|---|
| 414 | DIVLTQSPAILSASPGEKVTMTCRASSS | Anti-CD20 Leu16 scFv |
| | VNYMDWYQKKPGSSPKPWIYATSNLA | entire sequence, with |
| | SGVPARFSGSGSGTSYSLTISRVEAEDA | Whitlow linker |
| | ATYYCQQWSFNPPTFGGGTKLEIKGSTS | |
| | GSGKPGSGEGSTKGEVQLQQSGAELVK | |
| | PGASVKMSCKASGYTFTSYNMHWVKQ | |
| | TPGQGLEWIGAIYPGNGDTSYNQKFKG | |
| | KATLTADKSSSTAYMQLSSLTSEDSAD | |
| | YYCARSNYYGSSYWFFDVWGAGTTVT | |
| | VSS | |
| 415 | DIVLTQSPAILSASPGEKVTMTCRASSS | Anti-CD20 Leu16 scFv |
| | VNYMDWYQKKPGSSPKPWIYATSNLA | light chain variable |
| | SGVPARFSGSGSGTSYSLTISRVEAEDA | region |
| | ATYYCQQWSFNPPTFGGGTKLEIK | |
| 416 | RASSSVNYMD | Anti-CD20 Leu16 scFv |
| | | light chain CDR1 |
| 417 | ATSNLAS | Anti-CD20 Leu16 scFv |
| | | light chain CDR2 |
| 418 | QQWSFNPPT | Anti-CD20 Leu16 scFv |
| | | light chain CDR3 |
| 419 | EVQLQQSGAELVKPGASVKMSCKASG | Anti-CD20 Leu16 scFv |
| | YTFTSYNMHWVKQTPGQGLEWIGAIYP | heavy chain |
| | GNGDTSYNQKFKGKATLTADKSSSTAY | |
| | MQLSSLTSEDSADYYCARSNYYGSSYW | |
| | FFDVWGAGTTVTVSS | |
| 420 | SYNMH | Anti-CD20 Leu16 scFv |
| | | heavy chain CDR1 |
| 421 | AIYPGNGDTSYNQKFKG | Anti-CD20 Leu16 scFv |
| | | heavy chain CDR2 |
| 422 | SNYYGSSYWFFDV | Anti-CD20 Leu16 scFv |
| | | heavy chain CDR3 |

In some embodiments, the hinge domain of the CD20 CAR comprises a CD8α hinge domain, for example, a human CD8α hinge domain. In some embodiments, the CD8α hinge domain comprises or consists of an amino acid sequence set forth in SEQ ID NO:378 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:378. In some embodiments, the hinge domain comprises a CD28 hinge domain, for example, a human CD28 hinge domain. In some embodiments, the CD28 hinge domain comprises or consists of an amino acid sequence set forth in SEQ ID NO:382 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:382. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:384 or SEQ ID NO:385, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:384 or SEQ ID NO:385. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:386 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:386.

In some embodiments, the transmembrane domain of the CD20 CAR comprises a CD8α transmembrane domain, for example, a human CD8α transmembrane domain. In some embodiments, the CD8α transmembrane domain comprises or consists of an amino acid sequence set forth in SEQ ID NO:387 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:387. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:388 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:388.

In some embodiments, the intracellular costimulatory domain of the CD20 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:390 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:390. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:391 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:391.

In some embodiments, the intracellular signaling domain of the CD20 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:392 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:392.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:414, the CD8α hinge domain of SEQ ID NO:381, the CD8α transmembrane domain of SEQ ID NO:387, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:414, the CD28 hinge domain of SEQ ID NO:381, the CD8α transmembrane domain of SEQ ID NO:387, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:414, the IgG4 hinge domain of SEQ ID NO:384 or SEQ ID NO:385, the CD8α transmembrane domain of SEQ ID NO:387, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:414, the CD8α hinge domain of SEQ ID NO:387, the CD28 transmembrane domain of SEQ ID NO:389, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:414, the CD28 hinge domain of SEQ ID NO:382, the CD28 transmembrane domain of SEQ ID NO:388, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:414, the IgG4 hinge domain of SEQ ID NO:384 or SEQ ID NO:385, the CD28 transmembrane domain of SEQ ID NO:388, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### c) CD22 CAR

In some embodiments, the CAR is a CD22 CAR (“CD22-CAR”), and in these embodiments, the polycistronic vector comprises an expression cassette that contains a nucleotide sequence encoding a CD22 CAR. CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR may comprise a signal peptide, an extracellular binding domain that specifically binds CD22, a hinge domain, a transmembrane domain, an intracellular costimulatory domain, and/or an intracellular signaling domain in tandem.

In some embodiments, the signal peptide of the CD22 CAR comprises a CD8α signal peptide. In some embodiments, the CD8α signal peptide comprises or consists of an amino acid sequence set forth in SEQ ID NO:378 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:378. In some embodiments, the signal peptide comprises an IgK signal peptide. In some embodiments, the IgK signal peptide comprises or consists of an amino acid sequence set forth in SEQ ID NO:379 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:379. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:380 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:380.

In some embodiments, the extracellular binding domain of the CD22 CAR is specific to CD22, for example, human CD22. The extracellular binding domain of the CD22 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD22 CAR is derived from an antibody specific to CD22, including, for example, SM03, inotuzumab, epratuzumab, moxetumomab, and pinatuzumab. In any of these embodiments, the extracellular binding domain of the CD22 CAR can comprise or consist of the V_{H}, the V_{L}, and/or one or more CDRs of any of the antibodies.

In some embodiments, the extracellular binding domain of the CD22 CAR comprises an scFv derived from the m971 monoclonal antibody (m971), which comprises the heavy chain variable region (V_{H}) and the light chain variable region (V_{L}) of m971 connected by a linker. In some embodiments, the linker is a 3xG₄S linker. In other embodiments, the Whitlow linker may be used instead. In some embodiments, the amino acid sequences of the entire m971-derived scFv (also referred to as m971 scFv) and its different portions are provided in Table 15 below. In some embodiments, the CD22-specific scFv comprises or consists of an amino acid sequence set forth in SEQ ID NO:423 or 432, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:423 or 432. In some embodiments, the CD22-specific scFv comprises or consists of a heavy chain variable region that comprises or consists of an amino acid sequence set forth in SEQ ID NO:424, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:424. In some embodiments, the CD22-specific scFv comprises or consists of a light chain variable region that comprises or consists of an amino acid sequence set forth in SEQ ID NO:428, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:428. In some embodiments, the CD22-specific scFv may comprise one or more CDRs having amino acid sequences set forth in SEQ ID NOs: 425-427 and 429-431, and 434-436, and 438-440. In some embodiments, the CD22-specific scFv may comprise a heavy chain with one or more CDRs having amino acid sequences set forth in SEQ ID NOs: 425-427 or 434-436. In some embodiments, the CD22-specific scFv may comprise a light chain with one or more CDRs having amino acid sequences set forth in SEQ ID NOs: 429-431 or 438-440. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

In some embodiments, the extracellular binding domain of the CD22 CAR comprises an scFv derived from m971-L7, which is an affinity matured variant of m971 with significantly improved CD22 binding affinity compared to the parental antibody m971 (improved from about 2 nM to less than 50 pM). In some embodiments, the scFv derived from m971-L7 comprises the V_{H} and the V_{L} of m971-L7 connected by a 3xG₄S linker. In other embodiments, the Whitlow linker may be used instead. In some embodiments, the amino acid sequences of the entire m971-L7-derived scFv (also referred to as m971-L7 scFv) and its different portions are provided in Table 15 below. In some embodiments, the CD22-specific scFv comprises or consists of an amino acid sequence set forth in SEQ ID NO:423 or 432, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:423, or 432. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

TABLE 15
Exemplary sequences of anti-CD22 scFv and components

| SEQ ID NO: | Amino Acid Sequence | Description |
|---|---|---|
| 423 | QVQLQQSGPGLVKPSQTLSLTCAISGDS | Anti-CD22 m971 scFv |
| | VSSNSAAWNWIRQSPSRGLEWLGRTYY | entire sequence, with |
| | RSKWYNDYAVSVKSRITINPDTSKNQFS | 3xG₄S linker |
| | LQLNSVTPEDTAVYYCAREVTGDLEDA | |
| | FDIWGQGTMVTVSSGGGGSGGGGSGG | |
| | GGSDIQMTQSPSSLSASVGDRVTITCRA | |
| | SQTIWSYLNWYQQRPGKAPNLLIYAAS | |
| | SLQSGVPSRFSGRGSGTDFTLTISSLQAE | |
| | DFATYYCQQSYSIPQTFGQGTKLEIK | |
| 424 | QVQLQQSGPGLVKPSQTLSLTCAISGDS | Anti-CD22 m971 scFv |
| | VSSNSAAWNWIRQSPSRGLEWLGRTYY | heavy chain variable |
| | RSKWYNDYAVSVKSRITINPDTSKNQFS | region |
| | LQLNSVTPEDTAVYYCAREVTGDLEDA | |
| | FDIWGQGTMVTVSS | |
| 425 | GDSVSSNSAA | Anti-CD22 m971 scFv |
| | | heavy chain CDR1 |
| 426 | TYYRSKWYN | Anti-CD22 m971 scFv |
| | | heavy chain CDR2 |
| 427 | AREVTGDLEDAFDI | Anti-CD22 m971 scFv |
| | | heavy chain CDR3 |
| 428 | DIQMTQSPSSLSASVGDRVTITCRASQTI | Anti-CD22 m971 scFv |
| | WSYLNWYQQRPGKAPNLLIYAASSLQS | light chain |
| | GVPSRFSGRGSGTDFTLTISSLQAEDFAT | |
| | YYCQQSYSIPQTFGQGTKLEIK | |
| 429 | QTIWSY | Anti-CD22 m971 scFv |
| | | light chain CDR1 |
| 430 | AAS | Anti-CD22 m971 scFv |
| | | light chain CDR2 |
| 431 | QQSYSIPQT | Anti-CD22 m971 scFv |
| | | light chain CDR3 |
| 432 | QVQLQQSGPGMVKPSQTLSLTCAISGD | Anti-CD22 m971-L7 scFv |
| | SVSSNSVAWNWIRQSPSRGLEWLGRTY | entire sequence, with |
| | YRSTWYNDYAVSMKSRITINPDTNKNQ | 3xG₄S linker |
| | FSLQLNSVTPEDTAVYYCAREVTGDLE | |
| | DAFDIWGQGTMVTVSSGGGGSGGGGS | |
| | GGGGSDIQMIQSPSSLSASVGDRVTITC | |
| | RASQTIWSYLNWYRQRPGEAPNLLIYA | |
| | ASSLQSGVPSRFSGRGSGTDFTLTISSLQ | |
| | AEDFATYYCQQSYSIPQTFGQGTKLEIK | |
| 433 | QVQLQQSGPGMVKPSQTLSLTCAISGD | Anti-CD22 m971-L7 scFv |
| | SVSSNSVAWNWIRQSPSRGLEWLGRTY | heavy chain variable |
| | YRSTWYNDYAVSMKSRITINPDTNKNQ | region |
| | FSLQLNSVTPEDTAVYYCAREVTGDLE | |
| | DAFDIWGQGTMVTVSS | |
| 434 | GDSVSSNSVA | Anti-CD22 m971-L7 scFv |
| | | heavy chain CDR1 |
| 435 | TYYRSTWYN | Anti-CD22 m971-L7 scFv |
| | | heavy chain CDR2 |
| 436 | AREVTGDLEDAFDI | Anti-CD22 m971-L7 scFv |
| | | heavy chain CDR3 |
| 437 | DIQMIQSPSSLSASVGDRVTITCRASQTI | Anti-CD22 m971-L7 scFv |
| | WSYLNWYRQRPGEAPNLLIYAASSLQS | light chain variable |
| | GVPSRFSGRGSGTDFTLTISSLQAEDFAT | region |
| | YYCQQSYSIPQTFGQGTKLEIK | |
| 438 | QTIWSY | Anti-CD22 m971-L7 scFv |
| | | light chain CDR1 |
| 439 | AAS | Anti-CD22 m971-L7 scFv |
| | | light chain CDR2 |
| 440 | QQSYSIPQT | Anti-CD22 m971-L7 scFv |
| | | light chain CDR3 |

In some embodiments, the extracellular binding domain of the CD22 CAR comprises immunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280(1): 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the hinge domain of the CD22 CAR comprises a CD8α hinge domain, for example, a human CD8α hinge domain. In some embodiments, the CD8α hinge domain comprises or consists of an amino acid sequence set forth in SEQ ID NO:381 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:381. In some embodiments, the hinge domain comprises a CD28 hinge domain, for example, a human CD28 hinge domain. In some embodiments, the CD28 hinge domain comprises or consists of an amino acid sequence set forth in SEQ ID NO:382 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:381. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:384 or SEQ ID NO:385, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:384 or SEQ ID NO:385. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:386 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:386.

In some embodiments, the transmembrane domain of the CD22 CAR comprises a CD8α transmembrane domain, for example, a human CD8α transmembrane domain. In some embodiments, the CD8α transmembrane domain comprises or consists of an amino acid sequence set forth in SEQ ID NO:387 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:387. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:388 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:388.

In some embodiments, the intracellular costimulatory domain of the CD22 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:390 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:390. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:391 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:391.

In some embodiments, the intracellular signaling domain of the CD22 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:392 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:392.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:423 or SEQ ID NO:432, the CD8α hinge domain of SEQ ID NO:381, the CD8α transmembrane domain of SEQ ID NO:387, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:423 or SEQ ID NO:432, the CD28 hinge domain of SEQ ID NO:382, the CD8α transmembrane domain of SEQ ID NO:387, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:423 or SEQ ID NO:432, the IgG4 hinge domain of SEQ ID NO:384 or SEQ ID NO:385, the CD8α transmembrane domain of SEQ ID NO:387, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:423 or SEQ ID NO:432, the CD8α hinge domain of SEQ ID NO:381, the CD28 transmembrane domain of SEQ ID NO:389, the 4-1BB costimulatory domain of SEQ ID NO:390, the CD3ζ signaling domain of SEQ ID NO:391, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:423 or SEQ ID NO:432, the CD28 hinge domain of SEQ ID NO:383, the CD28 transmembrane domain of SEQ ID NO:389, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:423 or SEQ ID NO:432, the IgG4 hinge domain of SEQ ID NO:384 or SEQ ID NO:385, the CD28 transmembrane domain of SEQ ID NO:388, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### d) BCMA CAR

In some embodiments, the CAR is a BCMA CAR (“BCMA-CAR”), and in these embodiments, the polycistronic vector comprises an expression cassette that contains a nucleotide sequence encoding a BCMA CAR. BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the BCMA CAR may comprise a signal peptide, an extracellular binding domain that specifically binds BCMA, a hinge domain, a transmembrane domain, an intracellular costimulatory domain, and/or an intracellular signaling domain in tandem.

In some embodiments, the signal peptide of the BCMA CAR comprises a CD8α signal peptide. In some embodiments, the CD8α signal peptide comprises or consists of an amino acid sequence set forth in SEQ ID NO:378 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:378. In some embodiments, the signal peptide comprises an IgK signal peptide. In some embodiments, the IgK signal peptide comprises or consists of an amino acid sequence set forth in SEQ ID NO:379 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:379. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:380 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:380.

In some embodiments, the extracellular binding domain of the BCMA CAR is specific to BCMA, for example, human BCMA. The extracellular binding domain of the BCMA CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BCMA CAR is derived from an antibody specific to BCMA, including, for example, belantamab, erlanatamab, teclistamab, LCAR-B38M, and ciltacabtagene. In any of these embodiments, the extracellular binding domain of the BCMA CAR can comprise or consist of the V_{H}, the V_{L}, and/or one or more CDRs of any of the antibodies.

In some embodiments, the extracellular binding domain of the BCMA CAR comprises an scFv derived from C11D5.3, a murine monoclonal antibody as described in Carpenter et al., Clin. Cancer Res. 19(8):2048-2060 (2013). See also PCT Application Publication No. WO2010/104949. The C11D5.3-derived scFv may comprise the heavy chain variable region (V_{H}) and the light chain variable region (V_{L}) of C11D5.3 connected by the Whitlow linker, the amino acid sequences of which is provided in Table 17 below. In some embodiments, the BCMA-specific extracellular binding domain comprises or consists of an amino acid sequence set forth in SEQ ID NO:441, 450, or 463, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 441. In some embodiments, the BCMA-specific extracellular binding domain comprises or consists of a heavy chain variable region comprising or consisting an amino acid sequence set forth in SEQ ID NO:446, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:446. In some embodiments, the BCMA-specific extracellular binding domain comprises or consists of a light chain variable region comprising or consisting an amino acid sequence set forth in SEQ ID NO:442, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 442. In some embodiments, the BCMA-specific extracellular binding domain may comprise one or more CDRs having amino acid sequences set forth in SEQ ID NOs: 443-445 and 447-449. In some embodiments, the BCMA-specific extracellular binding domain may comprise a light chain with one or more CDRs having amino acid sequences set forth in SEQ ID NOs: 443-445. In some embodiments, the BCMA-specific extracellular binding domain may comprise a heavy chain with one or more CDRs having amino acid sequences set forth in SEQ ID NOs: 447-449. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from another murine monoclonal antibody, C12A3.2, as described in Carpenter et al., Clin. Cancer Res. 19(8):2048-2060 (2013) and PCT Application Publication No. WO2010/104949, the amino acid sequence of which is also provided in Table 16 below. In some embodiments, the BCMA-specific extracellular binding domain comprises or consists of an amino acid sequence set forth in SEQ ID NO:450, 450, 459, or 463, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 450. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises a murine monoclonal antibody with high specificity to human BCMA, referred to as BB2121 in Friedman et al., Hum. Gene Ther. 29(5):585-601 (2018)). See also, PCT Application Publication No. WO2012163805.

In some embodiments, the extracellular binding domain of the BCMA CAR comprises single variable fragments of two heavy chains (VHH) that can bind to two epitopes of BCMA as described in Zhao et al., J. Hematol. Oncol. 11(1):141 (2018), also referred to as LCAR-B38M. See also, PCT Application Publication No. WO2018/028647.

In some embodiments, the extracellular binding domain of the BCMA CAR comprises a fully human heavy-chain variable domain (FHVH) as described in Lam et al., Nat. Commun. 11(1):283 (2020), also referred to as FHVH33. See also, PCT Application Publication No. WO2019/006072. The amino acid sequences of FHVH33 and its CDRs are provided in Table 16 below. In some embodiments, the BCMA-specific extracellular binding domain comprises or consists of an amino acid sequence set forth in SEQ ID NO:459 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 459. In some embodiments, the BCMA-specific extracellular binding domain may comprise one or more CDRs having amino acid sequences set forth in SEQ ID NOs: 460-462. In any of these embodiments, the BCMA-specific extracellular binding domain may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from CT103A (or CAR0085) as described in U.S. Pat. No. 11,026,975 B2, the amino acid sequence of which is provided in Table 16 below. In some embodiments, the BCMA-specific extracellular binding domain comprises or consists of an amino acid sequence set forth in SEQ ID NO:463, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 463. In some embodiments, the BCMA-specific extracellular binding domain may comprise one or more CDRs having amino acid sequences set forth in SEQ ID NOs: 465-497 and 469-471. In some embodiments, the BCMA-specific extracellular binding domain may comprise a light chain with one or more CDRs having amino acid sequences set forth in SEQ ID NOs: 465-497. In some embodiments, the BCMA-specific extracellular binding domain may comprise a heavy chain with one or more CDRs having amino acid sequences set forth in SEQ ID NOs: 469-471. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

Additionally, CARs and binders directed to BCMA have been described in U.S. Application Publication Nos. 2020/0246381 A1 and 2020/0339699 A1, the entire contents of each of which are incorporated by reference herein.

TABLE 16
Exemplary sequences of anti-BCMA binder and components

| SEQ ID NO: | Amino Acid Sequence | Description |
|---|---|---|
| 441 | DIVLTQSPASLAMSLGKRATISCRASES | Anti-BCMA C11D5.3 |
| | VSVIGAHLIHWYQQKPGQPPKLLIYLAS | scFv entire sequence, |
| | NLETGVPARFSGSGSGTDFTLTIDPVEE | with Whitlow linker |
| | DDVAIYSCLQSRIFPRTFGGGTKLEIKGS | |
| | TSGSGKPGSGEGSTKGQIQLVQSGPELK | |
| | KPGETVKISCKASGYTFTDYSINWVKR | |
| | APGKGLKWMGWINTETREPAYAYDFR | |
| | GRFAFSLETSASTAYLQINNLKYEDTAT | |
| | YFCALDYSYAMDYWGQGTSVTVSS | |
| 442 | DIVLTQSPASLAMSLGKRATISCRASES | Anti-BCMA C11D5.3 |
| | VSVIGAHLIHWYQQKPGQPPKLLIYLAS | scFv light chain variable |
| | NLETGVPARFSGSGSGTDFTLTIDPVEE | region |
| | DDVAIYSCLQSRIFPRTFGGGTKLEIK | |
| 443 | RASESVSVIGAHLIH | Anti-BCMA C11D5.3 |
| | | scFv light chain CDR1 |
| 444 | LASNLET | Anti-BCMA C11D5.3 |
| | | scFv light chain CDR2 |
| 445 | LQSRIFPRT | Anti-BCMA C11D5.3 |
| | | scFv light chain CDR3 |
| 446 | QIQLVQSGPELKKPGETVKISCKASGYT | Anti-BCMA C11D5.3 |
| | FTDYSINWVKRAPGKGLKWMGWINTE | scFv heavy chain variable |
| | TREPAYAYDFRGRFAFSLETSASTAYLQ | region |
| | INNLKYEDTATYFCALDYSYAMDYWG | |
| | QGTSVTVSS | |
| 447 | DYSIN | Anti-BCMA C11D5.3 |
| | | scFv heavy chain CDR1 |
| 448 | WINTETREPAYAYDFRG | Anti-BCMA C11D5.3 |
| | | scFv heavy chain CDR2 |
| 449 | DYSYAMDY | Anti-BCMA C11D5.3 |
| | | scFv heavy chain CDR3 |
| 450 | DIVLTQSPPSLAMSLGKRATISCRASESV | Anti-BCMA C12A3.2 |
| | TILGSHLIYWYQQKPGQPPTLLIQLASN | scFv entire sequence, |
| | VQTGVPARFSGSGSRTDFTLTIDPVEED | with Whitlow linker |
| | DVAVYYCLQSRTIPRTFGGGTKLEIKGS | |
| | TSGSGKPGSGEGSTKGQIQLVQSGPELK | |
| | KPGETVKISCKASGYTFRHYSMNWVK | |
| | QAPGKGLKWMGRINTESGVPIYADDFK | |
| | GRFAFSVETSASTAYLVINNLKDEDTAS | |
| | YFCSNDYLYSLDFWGQGTALTVSS | |
| 451 | DIVLTQSPPSLAMSLGKRATISCRASESV | Anti-BCMA C12A3.2 |
| | TILGSHLIYWYQQKPGQPPTLLIQLASN | scFv light chain variable |
| | VQTGVPARFSGSGSRTDFTLTIDPVEED | region |
| | DVAVYYCLQSRTIPRTFGGGTKLEIK | |
| 452 | RASESVTILGSHLIY | Anti-BCMA C12A3.2 |
| | | scFv light chain CDR1 |
| 453 | LASNVQT | Anti-BCMA C12A3.2 |
| | | scFv light chain CDR2 |
| 454 | LQSRTIPRT | Anti-BCMA C12A3.2 |
| | | scFv light chain CDR3 |
| 455 | QIQLVQSGPELKKPGETVKISCKASGYT | Anti-BCMA C12A3.2 |
| | FRHYSMNWVKQAPGKGLKWMGRINTE | scFv heavy chain variable |
| | SGVPIYADDFKGRFAFSVETSASTAYLV | region |
| | INNLKDEDTASYFCSNDYLYSLDFWGQ | |
| | GTALTVSS | |
| 456 | HYSMN | Anti-BCMA C12A3.2 |
| | | scFv heavy chain CDR1 |
| 457 | RINTESGVPIYADDFKG | Anti-BCMA C12A3.2 |
| | | scFv heavy chain CDR2 |
| 458 | DYLYSLDF | Anti-BCMA C12A3.2 |
| | | scFv heavy chain CDR3 |
| 459 | EVQLLESGGGLVQPGGSLRLSCAASGF | Anti-BCMA FHVH33 |
| | TFSSYAMSWVRQAPGKGLEWVSSISGS | entire sequence |
| | GDYIYYADSVKGRFTISRDISKNTLYLQ | |
| | MNSLRAEDTAVYYCAKEGTGANSSLA | |
| | DYRGQGTLVTVSS | |
| 460 | GFTFSSYA | Anti-BCMA FHVH33 |
| | | CDR1 |
| 461 | ISGSGDYI | Anti-BCMA FHVH33 |
| | | CDR2 |
| 462 | AKEGTGANSSLADY | Anti-BCMA FHVH33 |
| | | CDR3 |
| 463 | DIQMTQSPSSLSASVGDRVTITCRASQSI | Anti-BCMA CT103A |
| | SSYLNWYQQKPGKAPKLLIYAASSLQS | scFv entire sequence, |
| | GVPSRFSGSGSGTDFTLTISSLQPEDFAT | with Whitlow linker |
| | YYCQQKYDLLTFGGGTKVEIKGSTSGS | |
| | GKPGSGEGSTKGQLQLQESGPGLVKPS | |
| | ETLSLTCTVSGGSISSSSYYWGWIRQPP | |
| | GKGLEWIGSISYSGSTYYNPSLKSRVTIS | |
| | VDTSKNQFSLKLSSVTAADTAVYYCAR | |
| | DRGDTILDVWGQGTMVTVSS | |
| 464 | DIQMTQSPSSLSASVGDRVTITCRASQSI | Anti-BCMA CT103A |
| | SSYLNWYQQKPGKAPKLLIYAASSLQS | scFv light chain variable |
| | GVPSRFSGSGSGTDFTLTISSLQPEDFAT | region |
| | YYCQQKYDLLTFGGGTKVEIK | |
| 465 | QSISSY | Anti-BCMA CT103A |
| | | scFv light chain CDR1 |
| 466 | AAS | Anti-BCMA CT103A |
| | | scFv light chain CDR2 |
| 467 | QQKYDLLT | Anti-BCMA CT103A |
| | | scFv light chain CDR3 |
| 468 | QLQLQESGPGLVKPSETLSLTCTVSGGS | Anti-BCMA CT103A |
| | ISSSSYYWGWIRQPPGKGLEWIGSISYS | scFv heavy chain variable |
| | GSTYYNPSLKSRVTISVDTSKNQFSLKL | region |
| | SSVTAADTAVYYCARDRGDTILDVWG | |
| | QGTMVTVSS | |
| 469 | GGSISSSSYY | Anti-BCMA CT103A |
| | | scFv heavy chain CDR1 |
| 470 | ISYSGST | Anti-BCMA CT103A |
| | | scFv heavy chain CDR2 |
| 471 | ARDRGDTILDV | Anti-BCMA CT103A |
| | | scFv heavy chain CDR3 |

In some embodiments, the hinge domain of the BCMA CAR comprises a CD8α hinge domain, for example, a human CD8α hinge domain. In some embodiments, the CD8α hinge domain comprises or consists of an amino acid sequence set forth in SEQ ID NO:381 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:381. In some embodiments, the hinge domain comprises a CD28 hinge domain, for example, a human CD28 hinge domain. In some embodiments, the CD28 hinge domain comprises or consists of an amino acid sequence set forth in SEQ ID NO:382 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:382. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:384 or SEQ ID NO:385, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:384 or SEQ ID NO:385. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:386 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:386.

In some embodiments, the transmembrane domain of the BCMA CAR comprises a CD8α transmembrane domain, for example, a human CD8α transmembrane domain. In some embodiments, the CD8α transmembrane domain comprises or consists of an amino acid sequence set forth in SEQ ID NO:387 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:387. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:388 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:388.

In some embodiments, the intracellular costimulatory domain of the BCMA CAR comprises a 4-1BB costimulatory domain, for example, a human 4-1BB costimulatory domain. In some embodiments, the 4-1BB costimulatory domain comprises or consists of an amino acid sequence set forth in SEQ ID NO:390 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:390. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:391 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:391.

In some embodiments, the intracellular signaling domain of the BCMA CAR comprises a CD3 zeta (ζ) signaling domain, for example, a human CD3ζ signaling domain. In some embodiments, the CD3ζ signaling domain comprises or consists of an amino acid sequence set forth in SEQ ID NO:392 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:392.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:381, the CD8α transmembrane domain of SEQ ID NO:387, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide (e.g., a CD8α signal peptide) as described.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:381, the CD8α transmembrane domain of SEQ ID NO:387, the CD28 costimulatory domain of SEQ ID NO:391, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide as described.

In some embodiments, the polycistronic vector comprises an expression cassette that contains a nucleotide sequence encoding a BCMA CAR as set forth in SEQ ID NO:472 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:472 (see Table 17). The encoded BCMA CAR has a corresponding amino acid sequence set forth in SEQ ID NO:473 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:473, with the following components: CD8α signal peptide, CT103A scFv (V_{L}-Whitlow linker-V_{H}), CD8α hinge domain, CD8α transmembrane domain, 4-1BB costimulatory domain, and CD3ζ signaling domain.

In some embodiments, the polycistronic vector comprises an expression cassette that contains a nucleotide sequence encoding a commercially available embodiment of BCMA CAR, including, for example, idecabtagene vicleucel (ide-cel, also called bb2121). In some embodiments, the polycistronic vector comprises an expression cassette that contains a nucleotide sequence encoding idecabtagene vicleucel or portions thereof. Idecabtagene vicleucel comprises a BCMA CAR with the following components: the BB2121 binder, CD8α hinge domain, CD8α transmembrane domain, 4-1BB costimulatory domain, and CD3ζ signaling domain.

TABLE 17
Exemplary sequences of BCMA CARs

| SEQ ID NO: | Sequence | Description |
|---|---|---|
| 472 | atggccttaccagtgaccgccttgctcctgccgctggccttgctgctcca | Exemplary BCMA |
| | cgccgccaggccggacatccagatgacccagtctccatcctccctgtct | CAR nucleotide |
| | gcatctgtaggagacagagtcaccatcacttgccgggcaagtcagagc | sequence |
| | attagcagctatttaaattggtatcagcagaaaccagggaaagcccctaa | |
| | gctcctgatctatgctgcatccagtttgcaaagtggggtcccatcaaggtt | |
| | cagtggcagtggatctgggacagatttcactctcaccatcagcagtctgc | |
| | aacctgaagattttgcaacttactactgtcagcaaaaatacgacctcctca | |
| | cttttggcggagggaccaaggttgagatcaaaggcagcaccagcggct | |
| | ccggcaagcctggctctggcgagggcagcacaaagggacagctgca | |
| | gctgcaggagtcgggcccaggactggtgaagccttcggagaccctgtc | |
| | cctcacctgcactgtctctggtggctccatcagcagtagtagttactactg | |
| | gggctggatccgccagcccccagggaaggggctggagtggattggg | |
| | agtatctcctatagtgggagcacctactacaacccgtccctcaagagtcg | |
| | agtcaccatatccgtagacacgtccaagaaccagttctccctgaagctga | |
| | gttctgtgaccgccgcagacacggcggtgtactactgcgccagagatc | |
| | gtggagacaccatactagacgtatggggtcagggtacaatggtcaccgt | |
| | cagctcattcgtgcccgtgttcctgcccgccaaacctaccaccacccctg | |
| | cccctagacctcccaccccagccccaacaatcgccagccagcctctgt | |
| | ctctgcggcccgaagcctgtagacctgctgccggcggagccgtgcaca | |
| | ccagaggcctggacttcgcctgcgacatctacatctgggcccctctggc | |
| | cggcacctgtggcgtgctgctgctgagcctggtgatcaccctgtactgc | |
| | aaccaccggaacaaacggggcagaaagaaactcctgtatatattcaaa | |
| | caaccatttatgagaccagtacaaactactcaagaggaagatggctgta | |
| | gctgccgatttccagaagaagaagaaggaggatgtgaactgagagtga | |
| | agttcagcagatccgccgacgcccctgcctaccagcagggacagaac | |
| | cagctgtacaacgagctgaacctgggcagacgggaagagtacgacgt | |
| | gctggacaagcggagaggccgggaccccgagatgggcggaaagcc | |
| | cagacggaagaacccccaggaaggcctgtataacgaactgcagaaag | |
| | acaagatggccgaggcctacagcgagatcggcatgaagggcgagcg | |
| | gaggcgcggcaagggccacgatggcctgtaccagggcctgagcacc | |
| | gccaccaaggacacctacgacgccctgcacatgcaggccctgccccc | |
| | caga | |
| 473 | MALPVTALLLPLALLLHAARPDIQMTQSPSSL | Exemplary BCMA |
| | SASVGDRVTITCRASQSISSYLNWYQQKPGKA | CAR amino acid |
| | PKLLIYAASSLQSGVPSRFSGSGSGTDFTLTISS | sequence |
| | LQPEDFATYYCQQKYDLLTFGGGTKVEIKGST | |
| | SGSGKPGSGEGSTKGQLQLQESGPGLVKPSET | |
| | LSLTCTVSGGSISSSSYYWGWIRQPPGKGLEWI | |
| | GSISYSGSTYYNPSLKSRVTISVDTSKNQFSLK | |
| | LSSVTAADTAVYYCARDRGDTILDVWGQGT | |
| | MVTVSSFVPVFLPAKPTTTPAPRPPTPAPTIAS | |
| | QPLSLRPEACRPAAGGAVHTRGLDFACDIYIW | |
| | APLAGTCGVLLLSLVITLYCNHRNKRGRKKLL | |
| | YIFKQPFMRPVQTTQEEDGCSCRFPEEEEGGC | |
| | ELRVKFSRSADAPAYQQGQNQLYNELNLGRR | |
| | EEYDVLDKRRGRDPEMGGKPRRKNPQEGLY | |
| | NELQKDKMAEAYSEIGMKGERRRGKGHDGL | |
| | YQGLSTATKDTYDALHMQALPPR | |

### 3. Gene Editing Enzymes

In some embodiments, the exogenous agent is or comprises a genome editing technology. In some embodiments, the exogenous agent is or comprises a heterologous protein that is associated with a genome editing technology. Any of a variety of agents associated with gene editing technologies can be included as the exogenous agent and/or heterologous protein, such as for delivery of gene editing machinery to a cell. In some embodiments, the gene editing technology can include systems involving nuclease, nickase, homing, integrase, transposase, recombinase, and/or reverse transcriptase activity. In some embodiments, the gene editing technologies can be used for knock-out or knock-down of genes. In some embodiments, the gene-editing technologies can be used for knock-in or integration of DNA into a region of the genome. In some embodiments, the exogenous agent and/or heterologous protein mediates single-strand breaks (SSB). In some embodiments, the exogenous agent and/or heterologous protein mediates double-strand breaks (DSB), including in connection with non-homologous end-joining (NHEJ) or homology-directed repair (HDR). In some embodiments, the exogenous agent and/or heterologous protein does not mediate SSB. In some embodiments, the exogenous agent and/or heterologous protein does not mediate DSB. In some embodiments, the exogenous agent and/or heterologous protein can be used for DNA base editing or prime-editing. In some embodiments, the exogenous agent and/or heterologous protein can be used for Programmable Addition via Site-specific Targeting Elements (PASTE).

In some embodiments, the exogenous agent is a nuclease for use in gene editing methods. In some embodiments, the nuclease is a zinc-finger nucleases (ZFNs), transcription-activator like effector nucleases (TALENs), or a CRISPR-associated protein-nuclease (Cas). In some embodiments, the Cas protein is selected from the group consisting of Cas3, Cas9, Cas10, Cas12, and Cas13. In some embodiments, the Cas is a Cas12a (also known as cpf1) from a Prevotella, Francisella novicida, Acidaminococcus sp., Lachnospiraceae bacterium, or Francisella bacteria. In some embodiments, the Cas is Cas9 from Streptococcus pyogenes. In some embodiments, the Cas is Cas9 from Streptococcus pyogenes (SpCas). In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9). In some embodiments, the Cas is a Cas12a (also known as Cpf1) from a Prevotella or Francisella bacteria, or the Cas is a Cas12b from a Bacillus, optionally Bacillus hisashii. In some embodiments, the Cas is a Cas12a (also known as cpf1) from a Prevotella, Francisella novicida, Acidaminococcus sp., Lachnospiraceae bacterium, or Francisella bacteria. In some embodiments, the nuclease is MAD7 or CasX. In some of any embodiments, the Cas is a Cas3, Cas13, CasMini, or any other Cas protein known in the art. See for example, Wang et al., Biosensors and Bioelectronics (165) 1: 2020, and Wu et al. Nature Reviews Chemistry (4) 441: 2020). The Cas9 nuclease can, in some embodiments, be a Cas9 or functional fragment thereof from any bacterial species. See, e.g., Makarova et al. Nature Reviews, Microbiology, 9: 467-477 (2011), including supplemental information, hereby incorporated by reference in its entirety.

In some embodiments, delivery of the nuclease is by a provided vector encoding the nuclease (e.g. Cas).

In some embodiments, the provided viral vector particles contain a nuclease protein and the nuclease protein is directly delivered to a target cell. Methods of delivering a nuclease protein include those as described, for example, in Cai et al. Elife, 2014, 3:e01911 and International patent publication No. WO2017068077. For instance, provided viral vector particles comprise one or more Cas protein(s), such as Cas9. In some embodiments, the nuclease protein (e.g. Cas, such as Cas 9) is engineered as a chimeric nuclease protein with a viral structural protein (e.g. GAG) for packaging into the viral vector particle (e.g. lentiviral vector particle). For instance, a chimeric Cas9-protein fusion with the structural GAG protein can be packaged inside a lentiviral vector particle. In some embodiments, the fusion protein is a cleavable fusion protein between (i) a viral structural protein (e.g. GAG) and (ii) a nuclease protein (e.g. Cas protein, such as Cas 9).

In some embodiments, the Cas is wild-type Cas9, which can site-specifically cleave double-stranded DNA, resulting in the activation of the double-strand break (DSB) repair machinery. DSBs can be repaired by the cellular Non-Homologous End Joining (NHEJ) pathway (Overballe-Petersen et al., 2013, Proc Natl Acad Sci USA, Vol. 110: 19860-19865), resulting in insertions and/or deletions (indels) which disrupt the targeted locus. Alternatively, if a donor template with homology to the targeted locus is supplied, the DSB may be repaired by the homology-directed repair (HDR) pathway allowing for precise replacement mutations to be made (Overballe-Petersen et al., 2013, Proc Natl Acad Sci USA, Vol. 110: 19860-19865; Gong et al., 2005, Nat. Struct Mol Biol, Vol. 12: 304-312). In some embodiments, the Cas is mutant form, known as Cas9 D10A, with only nickase activity. This means that Cas9D10A cleaves only one DNA strand, and does not activate NHEJ. Instead, when provided with a homologous repair template, DNA repairs are conducted via the high-fidelity HDR pathway only, resulting in reduced indel mutations (Cong et al., 2013, Science, Vol. 339: 819-823; Jinek et al., 2012, Science, Vol. 337: 816-821; Qi et al., 2013 Cell, Vol. 152: 1173-1183). Cas9D10A is even more appealing in terms of target specificity when loci are targeted by paired Cas9 complexes designed to generate adjacent DNA nicks (Ran et al., 2013, Cell, Vol. 154: 1380-1389). In some embodiments, the Cas is a nuclease-deficient Cas9 (Qi et al., 2013 Cell, Vol. 152: 1173-1183). For instance, mutations H840A in the HNH domain and D10A in the RuvC domain inactivate cleavage activity, but do not prevent DNA binding. Therefore, this variant can be used to target in a sequence-specific manner any region of the genome without cleavage. Instead, by fusing with various effector domains, dCas9 can be used either as a gene silencing or activation tools. Furthermore, it can be used as a visualization tool by coupling the guide RNA or the Cas9 protein to a fluorophore or a fluorescent protein. In some embodiments, the Cas protein comprises one or more mutations such that the Cas protein is converted into a nickase that is able to cleave only one strand of a double stranded DNA molecule (e.g., a SSB). In some embodiments, the Cas protein is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. In some embodiments, the Cas protein is Cas9. In some embodiments, the Cas9 is from a bacteria selected from the group consisting of Streptococcus pyogenes, Staphylococcus aureus, Neisseria meningitides, Campylobacter jejuni, and Streptococcus thermophilis. In some embodiments, the Cas9 is from Streptococcus pyogenes. In some embodiments, the Cas9 is from Streptococcus pyogenes and comprises one or more mutations in the RuvC I, RuvC II, or RuvC III motifs. In some embodiments, the Cas9 is from Streptococcus pyogenes and comprises a D10A mutation in the RuvC I motif. In some embodiments, the Cas9 is from Streptococcus pyogenes and comprises one or more mutations in the HNH catalytic domain. In some embodiments, the Cas9 is from Streptococcus pyogenes and comprises one or more mutations in the HNH catalytic domain selected from the group consisting of H840A, H854A, and H863A. In some embodiments, the Cas9 is from Streptococcus pyogenes and comprises a H840A mutation in the HNH catalytic domain. In some embodiments, the Cas9 is from Streptococcus pyogenes and comprises a mutation selected from the group consisting of D10A, H840A, H854A, and H863A.

In some embodiments, the Cas protein is selected from the group consisting of Cas3, Cas9, Cas10, Cas12, and Cas13. In particular embodiments, the nuclease is a Cas nuclease, such as Cas9. In some embodiments, delivery of the CRISPR/Cas can be used to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. In some embodiments, the one or more agent(s) (e.g., the heterologous protein) capable of inducing a DSB comprise Cas9 or a functional fragment thereof, and a first guide RNA, e.g., a first sgRNA, and a second guide RNA, e.g., a second sgRNA. The guide RNA, e.g., the first guide RNA or the second guide RNA, in some embodiments, binds to the recombinant nuclease and targets the recombinant nuclease to a specific location within the target gene such as at a location within the sense strand or the antisense strand of the target gene that is or includes the cleavage site. In some embodiments, the recombinant nuclease is a Cas protein from any bacterial species, or is a functional fragment thereof. In some embodiments, the Cas protein is Cas9 nuclease. Cas9 can, in some embodiments, be a Cas9 or functional fragment thereof from any bacterial species. See, e.g., Makarova et al. Nature Reviews, Microbiology, 9: 467-477 (2011), including supplemental information, hereby incorporated by reference in its entirety. In some embodiments, the Cas9 is from Streptococcus pyogenes (SpCas9). In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9).

In some embodiments, the Cas9 is from Streptococcus pyogenes (SpCas9) and comprises one or more mutations in the RuvC catalytic domain or the HNH catalytic domain. In some embodiments, the one or more mutations in the RuvC catalytic domain or the HNH catalytic domain inactivates the catalytic activity of the domain. In some embodiments, the recombinant nuclease has RuvC activity but does not have HNH activity. In some embodiments, the recombinant nuclease does not have RuvC activity but does have HNH activity. In some embodiments, the Cas9 is from Streptococcus pyogenes (SpCas9) and comprises one or more mutations selected from the group consisting of D10A, H840A, H854A, and H863A. In some embodiments, the Cas9 is from Streptococcus pyogenes (SpCas9) and comprises one or more mutations in the RuvC I, RuvC II, or RuvC III motifs. In some embodiments, the Cas9 is from Streptococcus pyogenes (SpCas9) and comprises a mutation in the RuvC I motif. In some embodiments, the Cas9 is from Streptococcus pyogenes (SpCas9) and comprises a D10A mutation in the RuvC I motif. In some embodiments, the Cas9 is from Streptococcus pyogenes (SpCas9) and comprises one or more mutations in the HNH catalytic domain. In some embodiments, the one or more mutations in the HNH catalytic domain is selected from the group consisting of H840A, H854A, and H863A. In some embodiments, the Cas9 is from Streptococcus pyogenes (SpCas9) and comprises a H840A mutation in the HNH catalytic domain. In some embodiments, the Cas9 is from Streptococcus pyogenes (SpCas9) and comprises a H840A mutation. In some embodiments, the Cas9 is from Streptococcus pyogenes (SpCas9) and comprises a D10A mutation. In some embodiments, the Cas9 is from Streptococcus pyogenes (SpCas9) and comprises one or more mutations selected from the group consisting of N497A, R661A, Q695A, and Q926A. In some embodiments, the Cas9 is from Streptococcus pyogenes (SpCas9) and comprises one or more mutations selected from the group consisting of R780A, K810A, K855A, H982A, K1003A, R1060A, and K848A. In some embodiments, the Cas9 is from Streptococcus pyogenes (SpCas9) and comprises one or more mutations selected from the group consisting of N692A, M694A, Q695A, and H698A. In some embodiments, the Cas9 is from Streptococcus pyogenes (SpCas9) and comprises one or more mutations selected from the group consisting of M495V, Y515N, K526E, and R661Q. In some embodiments, the Cas9 is from Streptococcus pyogenes (SpCas9) and comprises one or more mutations selected from the group consisting of F539S, M7631, and K890N. In some embodiments, the Cas9 is from Streptococcus pyogenes (SpCas9) and comprises one or more mutations selected from the group consisting of E480K, E543D, E1219V, A262T, S4091, M6941, E108G, S217A.

In some embodiments, the Cas9 is from Streptococcus pyogenes (SaCas9). In some embodiments, the SaCas9 is wild type SaCas9. In some embodiments, the SaCas9 comprises one or more mutations in REC3 domain. In some embodiments, the SaCas9 comprises one or more mutations in REC1 domain. In some embodiments, the SaCas9 comprises one or more mutations selected from the group consisting of N260D, N260Q, N260E, Q414A, Q414L. In some embodiments, the SaCas9 comprises one or more mutations in the recognition lobe. In some embodiments, the SaCas9 comprises one or more mutations selected from the group consisting of R245A, N413A, N419A. In some embodiments, the SaCas9 comprises one or more mutations in the RuvC-III domain. In some embodiments, the SaCas9 comprises a R654A mutation.

In some embodiments, the Cas protein is Cas12. In some embodiments, the Cas protein is Cas12a (i.e. cpf1). In some embodiments, the Cas12a is from the group consisting of Francisella novicida U112 (FnCas12a), Acidaminococcus sp. BV3L6 (AsCas12a), Moraxella bovoculi AAX11_00205 (Mb3Cas12a), Lachnospiraceae bacterium ND2006 (LbCas12a), Thiomicrospira sp. Xs5 (TsCas12a), Moraxella bovoculi AAX08_00205 (Mb2Cas12a), and Butyrivibrio sp. NC3005 (BsCas12a). In some embodiments, the Cas12a recognizes a T-rich 5′ protospacer adjacent motif (PAM). In some embodiments, the Cas12a processes its own crRNA without requiring a transactivating crRNA (tracrRNA). In some embodiments, the Cas12a processes both RNase and DNase activity. In some embodiments, the Cas12a is a split Cas12a platform, consisting of N-terminal and C-terminal fragments of Cas12a. In some embodiments, the split Cas12a platform is from Lachnospiraceae bacterium.

In some embodiments, the lipid particle further comprises a polynucleotide per se, i.e. a polynucleotide that does not encode for a heterologous protein. In some embodiments, the polynucleotide per se is associated with a gene editing system. For example, a lipid particle may comprise a guide RNA (gRNA), such as a single guide RNA (sgRNA).

In some embodiments, the one or more agent(s) (e.g., one or more exogenous agent and/or heterologous protein) comprise, or are used in combination with, a guide RNA, e.g., single guide RNA (sgRNA), for inducing a DSB at the cleavage site. In some embodiments, the one or more agent(s) comprise, or are used in combination with, more than one guide RNA, e.g., a first sgRNA and a second sgRNA, for inducing a DSB at the cleavage site through a SSB on each strand. In some embodiments, the one or more agent(s) (e.g., the heterologous protein) can be used in combination with a donor template, e.g., a single-stranded DNA oligonucleotide (ssODN), for HDR-mediated integration of the donor template into the target gene, such as at the targeting sequence. In some embodiments, the one or more agent(s) (e.g., one or more exogenous agent and/or heterologous protein) can be used in combination with a donor template, e.g., an ssODN, and a guide RNA, e.g., a sgRNA, for HDR-mediated integration of the donor template into the target gene, such as at the targeting sequence. In some embodiments, the one or more agent(s) (e.g., one or more exogenous agent and/or heterologous protein) can be used in combination with a donor template, e.g., an ssODN, and a first guide RNA, e.g., a first sgRNA, and a second guide RNA, e.g., a second sgRNA, for HDR-mediated integration of the donor template into the target gene, such as at the targeting sequence.

In particular embodiments, the genome-modifying agent is a Cas protein, such as Cas9. In some embodiments, delivery of the CRISPR/Cas can be used to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. In some embodiments, a dCas9 version of the CRISPR/Cas9 system can be used to target protein domains for transcriptional regulation, epigenetic modification, and microscopic visualization of specific genome loci.

In some embodiments, the genome-modifying agent, e.g., Cas9, is targeted to the cleavage site by interacting with a guide RNA, e.g., sgRNA, that hybridizes to a DNA sequence that immediately precedes a Protospacer Adjacent Motif (PAM) sequence. In general, a guide RNA, e.g., sgRNA, is any nucleotide sequence comprising a sequence, e.g., a crRNA sequence, that has sufficient complementarity with a target gene sequence to hybridize with the target gene sequence at the cleavage site and direct sequence-specific binding of the recombinant nuclease to a portion of the target gene that includes the cleavage site. Full complementarity (100%) is not necessarily required, so long as there is sufficient complementarity to cause hybridization and promote formation of a complex, e.g., CRISPR complex, that includes the recombinant nuclease, e.g., Cas9, and the guide RNA, e.g., sgRNA. In some embodiments, the cleavage site is situated at a site within the target gene that is homologous to the sequence of the guide RNA, e.g., sgRNA. In some embodiments, the cleavage site is situated approximately 3 nucleotides upstream of the PAM sequence. In some embodiments, the cleavage site is situated approximately 3 nucleotides upstream of the juncture between the guide RNA and the PAM sequence. In some embodiments, the cleavage site is situated 3 nucleotides upstream of the PAM sequence. In some embodiments, the cleavage site is situated 4 nucleotides upstream of the PAM sequence.

In some embodiments, the one or more agent(s) (e.g., one or more exogenous agent and/or heterologous protein) capable of inducing a DSB comprise a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fokl nuclease domain. In some embodiments, the TAL effector DNA binding domain is engineered to target a specific target sequence, e.g., a portion of a target gene that includes a cleavage site.

In some embodiments, the fusion protein is a zinc finger nuclease (ZFN) comprising a zinc finger DNA binding domain and a DNA cleavage domain. In some embodiments, the DNA cleavage domain is a Fokl nuclease domain. In some embodiments, the zinc finger DNA binding domain is engineered to target a specific target sequence, e.g., a portion of a target gene, that includes a cleavage site, such as the targeting sequence.

In some embodiments, the provided lipid particles can be for use in a method to deliver an exogenous agent which involves introducing, into a cell, one or more agent(s) (e.g., one or more exogenous agent and/or heterologous protein) capable of inducing a SSB at a cleavage site within the sense strand and a SSB at a cleavage site within the antisense strand of an endogenous target gene in the cell.

In some embodiments, the cleavage site in the sense strand is less than 400, less than 350, less than 300, less than 250, less than 200, less than 175, less than 150, less than 125, less than 100, less than 90, less than 80, less than 75, less than 70, less than 65, less than 60, less than 55, less than 50, less than 45, less than 40, or less than 35 nucleotides from the nucleotide that is complementary to the cleavage site in the antisense strand. In some embodiments, the cleavage site in the antisense strand is less than 400, less than 350, less than 300, less than 250, less than 200, less than 175, less than 150, less than 125, less than 100, less than 90, less than 80, less than 75, less than 70, less than 65, less than 60, less than 55, less than 50, less than 45, less than 40, or less than 35 nucleotides from the nucleotide that is complementary to the cleavage site in the sense strand. In some embodiments, the cleavage site in the sense strand is between 20 and 400, 20 and 350, 20 and 300, 20 and 250, 20 and 200, 20 and 150, 20 and 125, 20 and 100, 20 and 90, 20 and 80, 20 and 70, 30 and 400, 30 and 350, 30 and 300, 30 and 250, 30 and 200, 30 and 150, 30 and 125, 30 and 100, 30 and 90, 30 and 80, 30 and 70, 40 and 400, 40 and 350, 40 and 300, 40 and 250, 40 and 200, 40 and 150, 40 and 125, 40 and 100, 40 and 90, 40 and 80, or 40 and 70 nucleotides from the nucleotide that is complementary to the cleavage site in the antisense strand. In some embodiments, the cleavage site in the antisense strand is between 20 and 400, 20 and 350, 20 and 300, 20 and 250, 20 and 200, 20 and 150, 20 and 125, 20 and 100, 20 and 90, 20 and 80, 20 and 70, 30 and 400, 30 and 350, 30 and 300, 30 and 250, 30 and 200, 30 and 150, 30 and 125, 30 and 100, 30 and 90, 30 and 80, 30 and 70, 40 and 400, 40 and 350, 40 and 300, 40 and 250, 40 and 200, 40 and 150, 40 and 125, 40 and 100, 40 and 90, 40 and 80, or 40 and 70 nucleotides from the nucleotide that is complementary to the cleavage site in the sense strand.

In some embodiments, the one or more agent(s) (e.g., one or more exogenous agent and/or heterologous protein) capable of inducing a SSB at a cleavage site within the sense strand and a SSB at a cleavage site within the antisense strand comprise a recombinant nuclease. In some embodiments, the recombinant nuclease includes a recombinant nuclease that induces the SSB in the sense strand, and a recombinant nuclease that induced the SSB in the antisense strand, and both of which recombinant nucleases are referred to as the recombinant nuclease. Accordingly, in some embodiments, the method involves introducing, into a cell, one or more agent(s) (e.g., the one or more exogenous agent and/or heterologous protein) comprising a recombinant nuclease for inducing a SSB at a cleavage site in the sense strand and a SSB at a cleavage site in the antisense strand within an endogenous target gene in the cell. Although, in some embodiments, it is described that “a” “the” recombinant nuclease induces a SSB in the antisense strand a SSB in the sense strand, it is to be understood that this includes situations where two of the same recombinant nuclease is used, such that one of the recombinant nuclease induces the SSB in the sense strand and the other recombinant nuclease induces the SSB in the antisense strand. In some embodiments, the recombinant nuclease that induces the SSB lacks the ability to induce a DSB by cleaving both strands of double stranded DNA.

In some embodiments, the one or more agent(s) capable of inducing a SSB comprise a recombinant nuclease and a first guide RNA, e.g., a first sgRNA, and a second guide RNA, e.g., a second sgRNA.

In some embodiments, the genome-modifying agent is a Cas protein, a transcription activator-like effector nuclease (TALEN), or a zinc finger nuclease (ZFN). In some embodiments, the recombinant nuclease is a Cas nuclease. In some embodiments, the recombinant nuclease is a TALEN. In some embodiments, the recombinant nuclease is a ZFN.

In some embodiments, the one or more agent(s) capable of inducing a SSB at a cleavage site within the sense strand and a SSB at a cleavage site within the antisense strand comprise a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fokl nuclease domain. In some embodiments, the TAL effector DNA binding domain is engineered to target a specific target sequence, e.g., a portion of a target gene that includes a cleavage site. In some embodiments, the fusion protein is a zinc finger nuclease (ZFN) comprising a zinc finger DNA binding domain and a DNA cleavage domain. In some embodiments, the DNA cleavage domain is a Fokl nuclease domain. In some embodiments, the zinc finger DNA binding domain is engineered to target a specific target sequence, e.g., a portion of a target gene that includes a cleavage site, such as the targeting sequence.

In some embodiments, the one or more agent(s) capable of inducing a SSB at a cleavage site within the sense strand and a SSB at a cleavage site within the antisense strand involve use of the CRISPR/Cas gene editing system. In some embodiments, the one or more agent(s) comprise a recombinant nuclease.

In some embodiments, the genome-modifying agent is a Cas protein. In some embodiments, the Cas protein comprises one or more mutations such that the Cas protein is converted into a nickase that lacks the ability to cleave both strands of a double stranded DNA molecule. In some embodiments, the Cas protein comprises one or more mutations such that the Cas protein is converted into a nickase that is able to cleave only one strand of a double stranded DNA molecule. For example, Cas9, which is normally capable of inducing a double strand break, can be converted into a Cas9 nickase, which is capable of inducing a single strand break, by mutating one of two Cas9 catalytic domains: the RuvC domain, which comprises the RuvC I, RuvC II, and RuvC III motifs, or the NHN domain. In some embodiments, the Cas protein comprises one or more mutations in the RuvC catalytic domain or the HNH catalytic domain. In some embodiments, the genome-modifying protein is a recombinant nuclease that has been modified to have nickase activity. In some embodiments, the recombinant nuclease cleaves the strand to which the guide RNA, e.g., sgRNA, hybridizes, but does not cleave the strand that is complementary to the strand to which the guide RNA, e.g., sgRNA, hybridizes. In some embodiments, the recombinant nuclease does not cleave the strand to which the guide RNA, e.g., sgRNA, hybridizes, but does cleave the strand that is complementary to the strand to which the guide RNA, e.g., sgRNA, hybridizes.

In some embodiments, the lipid particle further comprises a guide RNA (gRNA), such as a single guide RNA (sgRNA). Thus, in some embodiments, the heterologous agent comprises a guide RNA (gRNA). In some embodiments, the gRNA is a single guide RNA (sgRNA).

In some embodiments, the genome-modifying protein, e.g., Cas9, is targeted to the cleavage site by interacting with a guide RNA, e.g., a first guide RNA, such as a first sgRNA, or a second guide RNA, such as a second sgRNA, that hybridizes to a DNA sequence on the sense strand or the antisense strand that immediately precedes a Protospacer Adjacent Motif (PAM) sequence.

In some embodiments, the genome-modifying agent, e.g., Cas9, is targeted to the cleavage site on the sense strand by interacting with a first guide RNA, e.g., first sgRNA, that hybridizes to a sequence on the sense strand that immediately precedes a PAM sequence. In some embodiments, the genome-modifying agent, e.g., Cas9, is targeted to the cleavage site on the antisense strand by interacting with a second guide RNA, e.g., second sgRNA, that hybridizes to a sequence on the antisense strand that immediately precedes a PAM sequence.

In some embodiments, the first guide RNA, e.g., first sgNA, that is specific to the sense strand of a target gene of interest is used to target the recombinant nuclease, e.g., Cas9, to induce a SSB at a cleavage site within the sense strand of the target gene. In some embodiments, the first guide RNA, e.g., first sgNA, that is specific to the antisense strand of a target gene of interest is used to target the recombinant nuclease, e.g., Cas9, to induce a SSB at a cleavage site within the antisense strand of the target gene.

In some embodiments, the second guide RNA, e.g., second sgNA, that is specific to the sense strand of a target gene of interest used to target the recombinant nuclease, e.g., Cas9, to induce a SSB at a cleavage site within the sense strand of the target gene. In some embodiments, the second guide RNA, e.g., second sgNA, that is specific to the antisense strand of a target gene of interest is used to target the recombinant nuclease, e.g., Cas9, to induce a SSB at a cleavage site within the antisense strand of the target gene.

In some embodiments, the first guide RNA, e.g., first sgNA, that is specific to the sense strand of a target gene of interest is used to target the recombinant nuclease, e.g., Cas9, to induce a SSB at a cleavage site within the sense strand of the target gene; and the second guide RNA, e.g., second sgNA, that is specific to the antisense strand of a target gene of interest is used to target the recombinant nuclease, e.g., Cas9, to induce a SSB at a cleavage site within the antisense strand of the target gene.

In some embodiments, the first guide RNA, e.g., first sgNA, that is specific to the antisense strand of a target gene of interest is used to target the recombinant nuclease, e.g., Cas9, to induce a SSB at a cleavage site within the antisense strand of the target gene; and the second guide RNA, e.g., second sgNA, that is specific to the sense strand of a target gene of interest is used to target the recombinant nuclease, e.g., Cas9, to induce a SSB at a cleavage site within the sense strand of the target gene. In general, a guide RNA, e.g., a first guide RNA, such as a first sgRNA, or a second guide RNA, such as a second sgRNA, is any nucleotide sequence comprising a sequence, e.g., a crRNA sequence, that has sufficient complementarity with a target gene sequence to hybridize with the target gene sequence at the cleavage site and direct sequence-specific binding of the recombinant nuclease to a portion of the target gene that includes the cleavage site. Full complementarity (100%) is not necessarily required, so long as there is sufficient complementarity to cause hybridization and promote formation of a complex, e.g., CRISPR complex, that includes the recombinant nuclease, e.g., Cas9, and the guide RNA, e.g., the first guide RNA, such as the first sgRNA, or the second guide RNA, such as the second sgRNA.

In some embodiments, the cleavage site is situated at a site within the target gene that is homologous to a sequence comprised within the guide RNA, e.g., sgRNA. In some embodiments, the cleavage site of the sense strand is situated at a site within the sense strand of the target gene that is homologous to a sequence comprised within the first guide RNA, e.g., the first sgRNA. In some embodiments, the cleavage site of the antisense strand is situated at a site within the antisense strand of the target gene that is homologous to a sequence comprised within the first guide RNA, e.g., the first sgRNA. In some embodiments, the cleavage site of the sense strand is situated at a site within the sense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the second guide RNA, e.g., the second sgRNA. In some embodiments, the cleavage site of the sense strand is situated at a site within the sense strand of the target gene that is homologous to a sequence comprised within the first guide RNA, e.g., the first sgRNA; and the cleavage site of the antisense strand is situated at a site within the antisense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the first guide RNA, e.g., the first sgRNA; and the cleavage site of the sense strand is situated at a site within the sense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the second guide RNA, e.g., the second sgRNA; and the cleavage site of the sense strand is situated at a site within the sense strand of the target gene that is homologous to a sequence comprised within the first guide RNA, e.g., the first sgRNA.

In some embodiments, the sense strand comprises the targeting sequence, and the targeting sequence includes the SNP and a protospacer adjacent motif (PAM) sequence. In some embodiments, the sense strand comprises the targeting sequence, and the targeting sequence includes the SNP and a protospacer adjacent motif (PAM) sequence; and the antisense strand comprises a sequence that is complementary to the targeting sequence and includes a PAM sequence. In some embodiments, the antisense strand comprises the targeting sequence, and the targeting sequence includes the SNP and a protospacer adjacent motif (PAM) sequence. In some embodiments, the antisense strand comprises the targeting sequence, and the targeting sequence includes the SNP and a protospacer adjacent motif (PAM) sequence; and the sense strand comprises a sequence that is complementary to the targeting sequence and includes a PAM sequence.

In some embodiments, the cleavage site on the sense strand and/or the antisense strand is situated approximately 3 nucleotides upstream of the PAM sequence. In some embodiments, the cleavage site on the sense strand and/or the antisense strand is situated approximately 3 nucleotides upstream of the juncture between the guide RNA and the PAM sequence. In some embodiments, the cleavage site on the sense strand and/or the antisense strand is situated 3 nucleotides upstream of the PAM sequence. In some embodiments, the cleavage site on the sense strand and/or the antisense strand is situated 4 nucleotides upstream of the PAM sequence.

In some embodiments, the PAM sequence that is recognized by a recombinant nuclease is in the sense strand. In some embodiments, the PAM sequence that is recognized by a recombinant nuclease is in the antisense strand. In some embodiments, the PAM sequence that is recognized by a recombinant nuclease is in the sense strand and is in the antisense strand. In some embodiments, the PAM sequence on the sense strand and the PAM sequence on the antisense strand are outwardly facing. In some embodiments, the PAM sequence on the sense strand and the PAM sequence on the antisense strand comprise the same nucleic acid sequence, which can be any PAM sequence disclosed herein. In some embodiments, the PAM sequence on the sense strand and the PAM sequence on the antisense strand each comprise a different nucleic acid sequence, each of which can be any of the PAM sequences disclosed herein.

In some embodiments, the PAM sequence that is recognized by a recombinant nuclease, e.g., Cas9, differs depending on the particular recombinant nuclease and the bacterial species it is from Methods for designing guide RNAs, e.g., sgRNAs, and their exemplary targeting sequences, e.g., crRNA sequences, can include those described in, e.g., International PCT Pub. Nos. WO2015/161276, WO2017/193107, and WO2017/093969. Exemplary guide RNA structures, including particular domains, are described in WO2015/161276, e.g., in FIGS. 1A-1G therein. Since guide RNA is an RNA molecule, it will comprise the base uracil (U), while any DNA encoding the guide RNA molecule will comprise the base thymine (T). In some embodiments, the guide RNA, e.g., sgRNA, comprises a CRISPR targeting RNA sequence (crRNA) and a trans-activating crRNA sequence (tracrRNA). In some embodiments, the first guide RNA, e.g., the first sgRNA, and the second guide RNA, e.g., the second sgRNA, each comprise a crRNA and a tracrRNA. In some embodiments, the guide RNA, e.g., sgRNA, is an RNA comprising, from 5′ to 3′: a crRNA sequence and a tracrRNA sequence. In some embodiments, each of the first guide RNA, e.g., first sgRNA, and the second guide RNA, e.g., second sgRNA, is an RNA comprising, from 5′ to 3′: a crRNA sequence and atracrRNA sequence. In some embodiments, the crRNA and tracrRNA do not naturally occur together in the same sequence.

In some embodiments, the crRNA comprises a nucleotide sequence that is homologous, e.g., is at least 80%, 85%, 90%, 95%, 96%, 97%, 98%, or 99% homologous, or is 100% homologous, to a portion of the target gene that includes the cleavage site. In some embodiments, the crRNA comprises a nucleotide sequence that is 100% homologous to a portion of the target gene that includes the cleavage site. In some embodiments, the portion of the target gene that includes the cleavage site is a portion of the sense strand of the target gene that includes the cleavage site. In some embodiments, the portion of the target gene that includes the cleavage site is a portion of the antisense strand of the target gene that includes the cleavage site.

In some embodiments, the sgRNA comprises a crRNA sequence that is homologous to a sequence in the target gene that includes the cleavage site. In some embodiments, the first sgRNA comprises a crRNA sequence that is homologous to a sequence in the sense strand of the target gene that includes the cleavage site; and/or the second sgRNA comprises a crRNA sequence that is homologous to a sequence in the antisense strand of the target gene that includes the cleavage site. In some embodiments, the first sgRNA comprises a crRNA sequence that is homologous to a sequence in the antisense strand of the target gene that includes the cleavage site; and/or the second sgRNA comprises a crRNA sequence that is homologous to a sequence in the sense strand of the target gene that includes the cleavage site.

In some embodiments, the crRNA sequence has 100% sequence identity to a sequence in the target gene that includes the cleavage site. In some embodiments, the crRNA sequence of the first sgRNA has 100% sequence identity to a sequence in the sense strand of the target gene that includes the cleavage site; and/or the crRNA sequence of the second sgRNA has 100% sequence identity to a sequence in the antisense strand of the target gene that includes the cleavage site. In some embodiments, the crRNA sequence of the first sgRNA has 100% sequence identity to a sequence in the antisense strand of the target gene that includes the cleavage site; and/or the crRNA sequence of the second sgRNA has 100% sequence identity to a sequence in the sense strand of the target gene that includes the cleavage site.

Guidance on the selection of crRNA sequences can be found, e.g., in Fu Y et al., Nat Biotechnol 2014 (doi: 10.1038/nbt.2808) and Sternberg S H et al., Nature 2014 (doi: 10.1038/nature13011). Examples of the placement of crRNA sequences within the guide RNA, e.g., sgRNA, structure include those described in WO2015/161276, e.g., in FIGS. 1A-1G therein.

Reference to “the crRNA” is to be understood as also including reference to the crRNA of the first sgRNA and the crRNA of the second sgRNA, each independently. Thus, embodiments referring to “the crRNA” is to be understood as independently referring to embodiments of (i) the crRNA, (ii) the crRNA of the first sgRNA, and (iii) the crRNA of the second sgRNA. In some embodiments, the crRNA is 15-27 nucleotides in length, i.e., the crRNA is 15, 16, 17, 18, 19, 20, 21, 22, 23, 24, 25, 26, or 27 nucleotides in length. In some embodiments, the crRNA is 18-22 nucleotides in length. In some embodiments, the crRNA is 19-21 nucleotides in length. In some embodiments, the crRNA is 20 nucleotides in length.

In some embodiments, the crRNA is homologous to a portion of a target gene that includes the cleavage site. In some embodiments, the crRNA is homologous to a portion of the sense strand of the target gene that includes the cleavage site. In some embodiments, the crRNA is homologous to a portion of the antisense strand of the target gene that includes the cleavage site. In some embodiments, the crRNA of the first sgRNA is homologous to a portion of the sense strand of the target gene that includes the cleavage site; and the crRNA of the second sgRNA is homologous to a portion of the antisense strand of the target gene that includes the cleavage site.

In some embodiments, the crRNA is homologous to a portion of the antisense strand of a target gene that includes the cleavage site. In some embodiments, the crRNA is homologous to a portion of the sense strand of the target gene that includes the cleavage site. In some embodiments, the crRNA of the first sgRNA is homologous to a portion of the antisense strand of the target gene that includes the cleavage site; and the crRNA of the second sgRNA is homologous to a portion of the sense strand of the target gene that includes the cleavage site.

In some embodiments, the crRNA is homologous to a portion of a target gene that includes the cleavage site, and is 15-27 nucleotides in length, i.e., the crRNA is 15, 16, 17, 18, 19, 20, 21, 22, 23, 24, 25, 26, or 27 nucleotides in length. In some embodiments, the portion of the target gene that includes the cleavage site is on the sense strand. In some embodiments, the portion of the target gene that includes the cleavage site is on the antisense strand.

In some embodiments, the crRNA is homologous to a portion, i.e., sequence, in the sense strand or the antisense strand of the target gene that includes the cleavage site and is immediately upstream of the PAM sequence.

In some embodiments, the tracrRNA sequence may be or comprise any sequence for tracrRNA that is used in any CRISPR/Cas9 system known in the art. Reference to “the tracrRNA” is to be understood as also including reference to the tracrRNA of the first sgRNA and the tracrRNA of the second sgRNA, each independently. Thus, embodiments referring to “the tracrRNA” is to be understood as independently referring to embodiments of (i) the tracrRNA, (ii) the tracrRNA of the first sgRNA, and (iii) the tracrRNA of the second sgRNA. Exemplary CRISPR/Cas9 systems, sgRNA, crRNA, and tracrRNA, and their manufacturing process and use include those described in, e.g., International PCT Pub. Nos. WO2015/161276, WO2017/193107 and WO2017/093969, and those described in, e.g., U.S. Patent Application Publication Nos. 20150232882, 20150203872, 20150184139, 20150079681, 20150073041, 20150056705, 20150031134, 20150020223, 20140357530, 20140335620, 20140310830, 20140273234, 20140273232, 20140273231, 20140256046, 20140248702, 20140242700, 20140242699, 20140242664, 20140234972, 20140227787, 20140189896, 20140186958, 20140186919, 20140186843, 20140179770, 20140179006, 20140170753, 20140093913, and 20140080216.

In some embodiments, the heterologous protein is associated with base editing. Base editors (BEs) are typically fusions of a Cas (“CRISPR-associated”) domain and a nucleobase modification domain (e.g., a natural or evolved deaminase, such as a cytidine deaminase that include APOBEC1 (“apolipoprotein B mRNA editing enzyme, catalytic polypeptide 1”), CDA (“cytidine deaminase”), and AID (“activation-induced cytidine deaminase”)) domains. In some cases, base editors may also include proteins or domains that alter cellular DNA repair processes to increase the efficiency and/or stability of the resulting single-nucleotide change.

In some aspects, currently available base editors include cytidine base editors (e.g., BE4) that convert target C·G to T·A and adenine base editors (e.g., ABE7.10) that convert target A·T to G·C. In some aspects, Cas9-targeted deamination was first demonstrated in connection with a Base Editor (BE) system designed to induce base changes without introducing double-strand DNA breaks. Further Rat deaminase APOBEC1 (rAPOBEC1) fused to deactivated Cas9 (dCas9) was used to successfully convert cytidines to thymidines upstream of the PAM of the sgRNA. In some aspects, this first BE system was optimized by changing the dCas9 to a “nickase” Cas9 D10A, which nicks the strand opposite the deaminated cytidine. Without being bound by theory, this is expected to initiate long-patch base excision repair (BER), where the deaminated strand is preferentially used to template the repair to produce a U:A base pair, which is then converted to T:A during DNA replication.

In some embodiments, the exogenous agent and/or heterologous protein is or encodes a base editor (e.g., a nucleobase editor). In some embodiments, the exogenous agent and/or heterologous protein is a nucleobase editor containing a first DNA binding protein domain that is catalytically inactive, a domain having base editing activity, and a second DNA binding protein domain having nickase activity, where the DNA binding protein domains are expressed on a single fusion protein or are expressed separately (e.g., on separate expression vectors). In some embodiments, the base editor is a fusion protein comprising a domain having base editing activity (e.g., cytidine deaminase or adenosine deaminase), and two nucleic acid programmable DNA binding protein domains (napDNAbp), a first comprising nickase activity and a second napDNAbp that is catalytically inactive, wherein at least the two napDNAbp are joined by a linker. In some embodiments, the base editor is a fusion protein that comprises a DNA domain of a CRISPR-Cas (e.g., Cas9) having nickase activity (nCas; nCas9), a catalytically inactive domain of a CRISPR-Cas protein (e.g., Cas9) having nucleic acid programmable DNA binding activity (dCas; e.g., dCas9), and a deaminase domain, wherein the dCas is joined to the nCas by a linker, and the dCas is immediately adjacent to the deaminase domain. In some embodiments, the base editor is a adenine-to-thymine or “ATBE” (or thymine-to-adenine or “TABE”) transversion base editors. Exemplary base editor and base editor systems include any as described in patent publication Nos. US20220127622, US20210079366, US20200248169, US20210093667, US20210071163, WO2020181202, WO2021158921, WO2019126709, WO2020181178, WO2020181195, WO2020214842, WO2020181193, which are hereby incorporated in their entirety.

In some embodiments, the exogenous agent and/or heterologous protein is one for use in target-primed reverse transcription (TPRT) or “prime editing”. In some embodiments, prime editing mediates targeted insertions, deletions, all 12 possible base-to-base conversions, and combinations thereof in human cells without requiring DSBs or donor DNA templates.

Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the prime editors search and locate the desired target site to be edited, and encode a replacement strand containing a desired edit which is installed in place of the corresponding target site endogenous DNA strand at the same time. For example, prime editing can be adapted for conducting precision CRISPR/Cas-based genome editing in order to bypass double stranded breaks. In some embodiments, the heterologous protein is or encodes for a Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. In some embodiments, the prime editor protein is paired with two prime editing guide RNAs (pegRNAs) that template the synthesis of complementary DNA flaps on opposing strands of genomic DNA, resulting in the replacement of endogenous DNA sequence between the PE-induced nick sites with pegRNA-encoded sequences.

In some embodiments, the exogenous agent and/or heterologous protein is or encodes for a primer editor that is a reverse transcriptase, or any DNA polymerase known in the art. Thus, in one aspect, the prime editor may comprise Cas9 (or an equivalent napDNAbp) which is programmed to target a DNA sequence by associating it with a specialized guide RNA (i.e., PEgRNA) containing a spacer sequence that anneals to a complementary protospacer in the target DNA. Such methods include any disclosed in Anzalone et al., (doi.org/10.1038/s41586-019-1711-4), or in PCT publication Nos. WO2020191248, WO2021226558, or WO2022067130, which are hereby incorporated in their entirety.

In some embodiments, the exogenous agent and/or heterologous protein is for use in Programmable Addition via Site-specific Targeting Elements (PASTE). In some aspects, PASTE is platform in which genomic insertion is directed via a CRISPR-Cas9 nickase fused to both a reverse transcriptase and serine integrase. As described in Ioannidi et al. (doi.org/10.1101/2021.11.01.466786), PASTE does not generate double stranded breaks, but allowed for integration of sequences as large as ~36 kb. In some embodiments, the serine integrase can be any known in the art. In some embodiments, the serine integrase has sufficient orthogonality such that PASTE can be used for multiplexed gene integration, simultaneously integrating at least two different genes at least two genomic loci. In some embodiments, PASTE has editing efficiencies comparable to or better than those of homology directed repair or non-homologous end joining based integration, with activity in nondividing cells and fewer detectable off-target events.

In some embodiments, the exogenous agent and/or heterologous protein is or encodes one or more polypeptides having an activity selected from the group consisting of: nuclease activity (e.g., programmable nuclease activity); nickase activity (e.g., programmable nickase activity); homing activity (e.g., programmable DNA binding activity); nucleic acid polymerase activity (e.g., DNA polymerase or RNA polymerase activity); integrase activity; recombinase activity; or base editing activity (e.g., cytidine deaminase or adenosine deaminase activity).

In some embodiments, delivery of the nuclease is by a provided vector encoding the nuclease (e.g. Cas).

In some embodiments, the provided lipid particles contain a nuclease protein and the nuclease protein is directly delivered to a target cell. Methods of delivering a nuclease protein include those as described, for example, in Cai et al. Elife, 2014, 3:e01911 and International patent publication No. WO2017068077. For instance, provided lipid particles comprise one or more Cas protein(s), such as Cas9. In some embodiments, the nuclease protein (e.g. Cas, such as Cas 9) is engineered as a chimeric nuclease protein with a viral structural protein (e.g. GAG) for packaging into the lipid particle (e.g. lentiviral vector particle, VLP, or gesicle). For instance, a chimeric Cas9-protein fusion with the structural GAG protein can be packaged inside a lipid particle. In some embodiments, the fusion protein is a cleavable fusion protein between (i) a viral structural protein (e.g. GAG) and (ii) a nuclease protein (e.g. Cas protein, such as Cas9). In some embodiments, the fusion protein is a cleavable fusion protein between (i) a viral matrix (MA) protein and (ii) a nuclease protein (e.g. Cas protein, such as Cas9). In some embodiments, the particle contains a nuclease protein (e.g., Cas protein, such as Cas 9) immediately downstream of the gag start codon.

In some embodiments, the provided lipid particles contain mRNA encoding a Cas nuclease (e.g., Cas9). In some embodiments, the provided lipid particles contain guide RNA (gRNA), such as a single guide RNA (sgRNA).

In some embodiments, a dCas9 version of the CRISPR/Cas9 system can be used to target protein domains for transcriptional regulation, epigenetic modification, and microscopic visualization of specific genome loci.

In some embodiments, the provided virus particles (e.g. lentiviral particles) containing a Cas nuclease (e.g. Cas9) further comprise, or is further complexed with, one or more CRISPR-Cas system guide RNA(s) for targeting a desired target gene. In some embodiments, the CRISPR guide RNAs are efficiently encapsulated in the CAS-containing viral particles. In some embodiments, the provided virus particles (e.g. lentiviral particles) further comprises, or is further complexed with a targeting nucleic acid.

### 4. Small Molecules

In some embodiments, the exogenous agent includes a small molecule, e.g., ions (e.g. Ca²⁺, Cl—, Fe²⁺), carbohydrates, lipids, reactive oxygen species, reactive nitrogen species, isoprenoids, signaling molecules, heme, polypeptide cofactors, electron accepting compounds, electron donating compounds, metabolites, ligands, and any combination thereof. In some embodiments the small molecule is a pharmaceutical that interacts with a target in the cell. In some embodiments the small molecule targets a protein in the cell for degradation. In some embodiments the small molecule targets a protein in the cell for degradation by localizing the protein to the proteasome. In some embodiments that small molecule is a proteolysis targeting chimera molecule (PROTAC).

In some embodiments, the exogenous agent includes a mixture of proteins, nucleic acids, or metabolites, e.g., multiple polypeptides, multiple nucleic acids, multiple small molecules; combinations of nucleic acids, polypeptides, and small molecules; ribonucleoprotein complexes (e.g. Cas9-gRNA complex); multiple transcription factors, multiple epigenetic factors, reprogramming factors (e.g. Oct4, Sox2, cMyc, and Klf4); multiple regulatory RNAs; and any combination thereof.

### V. PHARMACEUTICAL COMPOSITIONS

Provided herein are compositions containing the lipid particles herein, including lipid particles containing a retargeted attachment protein comprising (i) a paramyxovirus envelope attachment protein; and (ii) a targeting moiety directed to a first target molecule expressed on the surface of a target cell, and at least one paramyxovirus fusion protein. The pharmaceutical compositions can include any of the described lipid particles.

Also provided herein are compositions comprising any of the lipid particles described herein.

The pharmaceutical compositions provided herein can include any of the described lipid particles.

The present disclosure also provides, in some aspects, a pharmaceutical composition comprising the composition described herein and pharmaceutically acceptable carrier.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular lipid particle and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

In some embodiments, the lipid particle meets a pharmaceutical or good manufacturing practices (GMP) standard. In some embodiments, the lipid particle is made according to good manufacturing practices (GMP). In some embodiments, the lipid particle has a pathogen level below a predetermined reference value, e.g., is substantially free of pathogens. In some embodiments, the lipid particle has a contaminant level below a predetermined reference value, e.g., is substantially free of contaminants. In some embodiments, the lipid particle has low immunogenicity.

In some embodiments, formulations of the pharmaceutical compositions described herein may be prepared by any method known or hereafter developed in the art of pharmacology. In some embodiments, preparatory methods include the step of bringing the active ingredient into association with a carrier or one or more other accessory ingredients, and then, if necessary or desirable, shaping or packaging the product into a desired single- or multi-dose unit.

In some embodiments, a “unit dose” is a discrete amount of the pharmaceutical composition comprising a predetermined amount of the active ingredient. In some embodiments, the amount of the active ingredient is generally equal to the dosage of the active ingredient that would be administered to a subject or a convenient fraction of such a dosage such as, for example, one-half or one-third of such a dosage. In some embodiments, the unit dosage form may be for a single daily dose or one of multiple daily doses (e.g., about 1 to 4 or more times per day). In some embodiments, when multiple daily doses are used, the unit dosage form may be the same or different for each dose.

In some embodiments, the lipid particle containing the variant NiV-G is a viral vector or virus-like particle (e.g., Section IV.A). In some embodiments, the compositions provided herein can be formulated in dosage units of genome copies (GC). Suitable method for determining GC have been described and include, e.g., qPCR or digital droplet PCR (ddPCR) as described in, e.g., M. Lock et al, Hu Gene Therapy Methods, Hum Gene Ther Methods 25(2):115-25. 2014, which is incorporated herein by reference. In some embodiments, the dosage of administration of a viral vector or virus-like particle is from about 10⁴ to about 10¹⁰ GC units, inclusive. In some embodiments, the dosage of administration of a viral vector or virus-like particle is from about 10⁹ to about 10¹⁵ GC units, inclusive. In some embodiments, the dosage of administration of a viral vector or virus-like particle is from about 10⁵ to about 10⁹ GC units, inclusive. In some embodiments, the dosage of administration of a viral vector or virus-like particle is from about 10⁶ to about 10⁹ GC units, inclusive. In some embodiments, the dosage of administration of a viral vector or virus-like particle is from about 10¹² to about 10¹⁴ GC units, inclusive. In some embodiments, the dosage of administration is 1.0×10⁹ GC units, 5.0×10⁹ GC units, 1.0×10¹⁰ GC units, 5.0×10¹⁰ GC units, 1.0×10¹¹ GC units, 5.0×10¹¹ GC units, 1.0×10¹² GC units, 5.0×10¹² GC units, or 1.0×10¹³ GC units, 5.0×10¹³ GC units, 1.0×10¹⁴ GC units, 5.0×10¹⁴ GC units, or 1.0×10¹⁵ GC units.

In some embodiments, the dosage of administration of a viral vector or virus-like particle is from about 10⁴ to about 10¹⁰ infectious units, inclusive. In some embodiments, the dosage of administration of a viral vector or virus-like particle is from about 10⁹ to about 10¹⁵ infectious units, inclusive. In some embodiments, the dosage of administration of a viral vector or virus-like particle is from about 10⁵ to about 10⁹ infectious units. In some embodiments, the dosage of administration of a viral vector or virus-like particle is from about 10⁶ to about 10⁹ infectious units. In some embodiments, the dosage of administration of a viral vector or virus-like particle is from about 10¹² to about 10¹⁴ infectious units, inclusive. In some embodiments, the dosage of administration is 1.0×10⁹ infectious units, 5.0×10⁹ infectious units, 1.0×10¹⁰ infectious units, 5.0×10¹⁰ infectious units, 1.0×10¹¹ infectious units, 5.0×10¹¹ infectious units, 1.0×10¹² infectious units, 5.0×10¹² infectious units, or 1.0×10¹³ infectious units, 5.0×10¹³ infectious units, 1.0×10¹⁴ infectious units, 5.0×10¹⁴ infectious units, or 1.0×10¹⁵ infectious units. The techniques available for quantifying infectious units are routine in the art and include viral particle number determination, fluorescence microscopy, and titer by plaque assay. For example, the number of adenovirus particles can be determined by measuring the absorbance at A260. Similarly, infectious units can also be determined by quantitative immunofluorescence of vector specific proteins using monoclonal antibodies or by plaque assay.

In some embodiments, methods that calculate the infectious units include the plaque assay, in which titrations of the virus are grown on cell monolayers and the number of plaques is counted after several days to several weeks. For example, the infectious titer is determined, such as by plaque assay, for example an assay to assess cytopathic effects (CPE). In some embodiments, a CPE assay is performed by serially diluting virus on monolayers of cells, such as HFF cells, that are overlaid with agarose. After incubation for a time period to achieve a cytopathic effect, such as for about 3 to 28 days, generally 7 to 10 days, the cells can be fixed and foci of absent cells visualized as plaques are determined. In some embodiments, infectious units can be determined using an endpoint dilution (TCID50) method, which determines the dilution of virus at which 50% of the cell cultures are infected and hence, generally, can determine the titer within a certain range, such as one log.

In some embodiments, the dosage of administration of a viral vector or virus-like particle is from about 10⁴ to about 10¹⁰ plaque forming units (pfu), inclusive. In some embodiments, the dosage of administration of a viral vector or virus-like particle is from about 10⁹ to about 10¹⁵ pfu, inclusive. In some embodiments, the dosage of administration of a viral vector or virus-like particle is from about 10⁵ to about 10⁹ pfu. In some embodiments, the dosage of administration of a viral vector or virus-like particle is from about 10⁶ to about 10⁹ pfu. In some embodiments, the dosage of administration of a viral vector or virus-like particle is from about 1012 to about 10¹⁴ pfu, inclusive. In some embodiments, the dosage of administration is 1.0×10⁹ pfu, 5.0×10⁹ pfu, 1.0×10¹⁰ pfu, 5.0×10¹⁰ pfu, 1.0×10¹¹ pfu, 5.0×10¹¹ pfu, 1.0×10¹² pfu, 5.0×10¹² pfu, or 1.0×10¹³ pfu, 5.0×10¹⁴ pfu, 1.0×10¹⁴ pfu, 5.0×10¹⁴ pfu, or 1.0×10¹⁵ pfu.

In some embodiments, the subject will receive a single injection. In some embodiments, administration can be repeated at daily/weekly/monthly intervals for an indefinite period and/or until the efficacy of the treatment has been established. As set forth herein, the efficacy of treatment can be determined by evaluating the symptoms and clinical parameters described herein and/or by detecting a desired response.

The exact amount of vehicle provided lipid particle required will vary from subject to subject, depending on the species, age, weight and general condition of the subject, the particular polynucleic acid, polypeptide, or vector used, its mode of administration etc. TAn appropriate amount can be determined by one of ordinary skill in the art using only routine experimentation given the teachings herein.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the lipid particles in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Injectables can be prepared in conventional forms, either as liquid solutions or suspensions, solid forms suitable for solution of suspension in liquid prior to injection, or as emulsions. As used herein, “parenteral administration” includes intradermal, intranasal, subcutaneous, intramuscular, intraperitoneal, intravenous and intratracheal routes, as well as a slow release or sustained release system such that a constant dosage is maintained.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

In some embodiments, vehicle formulations may comprise cyroprotectants. As used herein, there term “cryoprotectant” refers to one or more agent that when combined with a given substance, helps to reduce or eliminate damage to that substance that occurs upon freezing. In some embodiments, cryoprotectants are combined with vector vehicles in order to stabilize them during freezing. In some aspects, Frozen storage of RNA between −20° C. and −80° C. may be advantageous for long term (e.g. 36 months) stability of polynucleotide. In some embodiments, the RNA species is mRNA. In some embodiments, cryoprotectants are included in vehicle formulations to stabilize polynucleotide through freeze/thaw cycles and under frozen storage conditions. Cryoprotectants of the provided embodiments may include, but are not limited to sucrose, trehalose, lactose, glycerol, dextrose, raffinose and/or mannitol. Trehalose is listed by the Food and Drug Administration as being generally regarded as safe (GRAS) and is commonly used in commercial pharmaceutical formulations.

The formulations to be used for in vivo administration are generally sterile. Sterility may be readily accomplished, e.g., by filtration through sterile filtration membranes.

### VI. METHODS OF USE

In some embodiments, the lipid particles provided herein or pharmaceutical compositions containing same can be administered to a subject, e.g. a mammal, e.g. a human. In such embodiments, the subject may be at risk of, may have a symptom of, or may be diagnosed with or identified as having, a particular disease or condition. In one embodiment, the subject has cancer. In one embodiment, the subject has an infectious disease. In some embodiments, the lipid particle contains nucleic acid sequences encoding an exogenous agent for treating the disease or condition in the subject. For example, the exogenous agent is one that targets or is specific for a protein of a neoplastic cells and the lipid particle is administered to a subject for treating a tumor or cancer in the subject. In another example, the exogenous agent is an inflammatory mediator or immune molecule, such as a cytokine, and lipid particle is administered to a subject for treating any condition in which it is desired to modulate (e.g. increase) the immune response, such as a cancer or infectious disease. In some embodiments, the lipid particle is administered in an effective amount or dose to effect treatment of the disease, condition or disorder. Provided herein are uses of any of the provided lipid particles in such methods and treatments, and in the preparation of a medicament in order to carry out such therapeutic methods. In some embodiments, the methods are carried out by administering the lipid particle or compositions comprising the same, to the subject having, having had, or suspected of having the disease or condition or disorder. In some embodiments, the methods thereby treat the disease or condition or disorder in the subject. Also provided herein are uses of any of the compositions, such as pharmaceutical compositions provided herein, for the treatment of a disease, condition or disorder associated with a particular gene or protein targeted by or provided by the exogenous agent.

In some embodiments, the provided methods or uses involve administration of a pharmaceutical composition comprising oral, inhaled, transdermal or parenteral (including intravenous, intratumoral, intraperitoneal, intramuscular, intracavity, intranodal and subcutaneous) administration. In some embodiments, the lipid particle may be administered alone or formulated as a pharmaceutical composition. In some embodiments, the lipid particle or compositions described herein can be administered to a subject, e.g., a mammal, e.g., a human. In some of any embodiments, the subject may be at risk of, may have a symptom of, or may be diagnosed with or identified as having, a particular disease or condition (e.g., a disease or condition described herein). In some embodiments, the disease is a disease or disorder.

In some embodiments, the lipid particles may be administered in the form of a unit-dose composition, such as a unit dose oral, parenteral, transdermal or inhaled composition. In some embodiments, the compositions are prepared by admixture and are adapted for oral, inhaled, transdermal or parenteral administration, and as such may be in the form of tablets, capsules, oral liquid preparations, powders, granules, lozenges, reconstitutable powders, injectable and infusible solutions or suspensions or suppositories or aerosols.

In some embodiments, the regimen of administration may affect what constitutes an effective amount. In some embodiments, the therapeutic formulations may be administered to the subject either prior to or after a diagnosis of disease. In some embodiments, several divided dosages, as well as staggered dosages may be administered daily or sequentially, or the dose may be continuously infused, or may be a bolus injection. In some embodiments, the dosages of the therapeutic formulations may be proportionally increased or decreased as indicated by the exigencies of the therapeutic or prophylactic situation.

In some embodiments, the administration of the compositions of the present invention to a subject, preferably a mammal, more preferably a human, may be carried out using known procedures, at dosages and for periods of time effective to prevent or treat disease. In some embodiments, an effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. In some embodiments, the dosage regimens may be adjusted to provide the optimum therapeutic response. In some embodiments, several divided doses may be administered daily or the dose may be proportionally reduced as indicated by the exigencies of the therapeutic situation. In some embodiments, the effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

In some embodiments, the compound may be administered to a subject as frequently as several times daily, or it may be administered less frequently, such as once a day, once a week, once every two weeks, once a month, or even less frequently, such as once every several months or even once a year or less. In some embodiments, the amount of compound dosed per day may be administered, in non-limiting examples, every day, every other day, every 2 days, every 3 days, every 4 days, or every 5 days. In some embodiments, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

In some embodiments, dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. In some embodiments, the physician or veterinarian can start doses of the compounds of the invention employed in the pharmaceutical composition at levels lower than that required in order to achieve the desired therapeutic effect and gradually increase the dosage until the desired effect is achieved.

In some embodiments, it is especially advantageous to formulate the compound in dosage unit form for ease of administration and uniformity of dosage. In some embodiments, dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. In some embodiments, the dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In some embodiments, the term “container” includes any receptacle for holding the pharmaceutical composition. In some embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. It should be understood that the instructions for use of the pharmaceutical composition may be contained on the packaging containing the pharmaceutical composition, and as such the instructions form an increased functional relationship to the packaged product. In some embodiments, instructions may contain information pertaining to the compound's ability to perform its intended function, e.g., treating or preventing a disease in a subject, or delivering an imaging or diagnostic agent to a subject.

In some embodiments, routes of administration of any of the compositions disclosed herein include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

In some of any embodiments, suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like.

In some embodiments, the lipid particle composition comprising an exogenous agent or cargo, may be used to deliver such exogenous agent or cargo to a cell tissue or subject. In some embodiments, delivery of a cargo by administration of a lipid particle composition described herein may modify cellular protein expression levels. In certain embodiments, the administered composition directs upregulation of (via expression in the cell, delivery in the cell, or induction within the cell) of one or more cargo (e.g., a polypeptide or mRNA) that provide a functional activity which is substantially absent or reduced in the cell in which the polypeptide is delivered. In some embodiments, the missing functional activity may be enzymatic, structural, or regulatory in nature. In some embodiments, the administered composition directs up-regulation of one or more polypeptides that increases (e.g., synergistically) a functional activity which is present but substantially deficient in the cell in which the polypeptide is upregulated. In some of any embodiments, the administered composition directs downregulation of (via expression in the cell, delivery in the cell, or induction within the cell) of one or more cargo (e.g., a polypeptide, siRNA, or miRNA) that repress a functional activity which is present or upregulated in the cell in which the polypeptide, siRNA, or miRNA is delivered. In some of any embodiments, the upregulated functional activity may be enzymatic, structural, or regulatory in nature. In some embodiments, the administered composition directs down-regulation of one or more polypeptides that decreases (e.g., synergistically) a functional activity which is present or upregulated in the cell in which the polypeptide is downregulated. In some embodiments, the administered composition directs upregulation of certain functional activities and downregulation of other functional activities.

In some of any embodiments, the lipid particle composition (e.g., one comprising mitochondria or DNA) mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the lipid particle composition comprises an exogenous protein), the effect lasts for less than 1, 2, 3, 4, 5, 6, or 7 days, 2, 3, or 4 weeks, or 1, 2, 3, 6, or 12 months.

In some of any embodiments, the lipid particle composition described herein is delivered ex-vivo to a cell or tissue, e.g., a human cell or tissue. In embodiments, the composition improves function of a cell or tissue ex-vivo, e.g., improves cell viability, respiration, or other function (e.g., another function described herein).

In some embodiments, the composition is delivered to an ex vivo tissue that is in an injured state (e.g., from trauma, disease, hypoxia, ischemia or other damage).

In some embodiments, the composition is delivered to an ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). In some embodiments, the composition is delivered to the tissue or organ before, during and/or after transplantation.

In some embodiments, the composition is delivered, administered or contacted with a cell, e.g., a cell preparation. In some embodiments, the cell preparation may be a cell therapy preparation (a cell preparation intended for administration to a human subject). In embodiments, the cell preparation comprises cells expressing a chimeric antigen receptor (CAR), e.g., expressing a recombinant CAR. The cells expressing the CAR may be, e.g., T cells, Natural Killer (NK) cells, cytotoxic T lymphocytes (CTL), regulatory T cells. In embodiments, the cell preparation is a neural stem cell preparation. In embodiments, the cell preparation is a mesenchymal stem cell (MSC) preparation. In embodiments, the cell preparation is a hematopoietic stem cell (HSC) preparation. In embodiments, the cell preparation is an islet cell preparation.

In some embodiments, the lipid particle compositions described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein).

In some embodiments, the source of lipid particles are from the same subject that is administered a lipid particle composition. In other embodiments, they are different. In some embodiments, the source of lipid particles and recipient tissue may be autologous (from the same subject) or heterologous (from different subjects). In some embodiments, the donor tissue for lipid particle compositions described herein may be a different tissue type than the recipient tissue. In some embodiments, the donor tissue may be muscular tissue and the recipient tissue may be connective tissue (e.g., adipose tissue). In other embodiments, the donor tissue and recipient tissue may be of the same or different type, but from different organ systems.

In some embodiments, the lipid particle composition described herein may be administered to a subject having a cancer, an autoimmune disease, an infectious disease, a metabolic disease, a neurodegenerative disease, or a genetic disease (e.g., enzyme deficiency). In some embodiments, the subject is in need of regeneration.

In some embodiments, the lipid particle is co-administered with an inhibitor of a protein that inhibits membrane fusion. For example, Suppressyn is a human protein that inhibits cell-cell fusion (Sugimoto et al., “A novel human endogenous retroviral protein inhibits cell-cell fusion” Scientific Reports 3: 1462 (DOI: 10.1038/srep01462)). In some embodiments, the lipid particle particles is co-administered with an inhibitor of sypressyn, e.g., a siRNA or inhibitory antibody.

### A. Mobilization

In some embodiments, also provided herein are methods of mobilizing hematopoietic cells, such as hematopoietic stem cells (HSCs), in connection with the provided methods of delivering a particle (e.g., lentiviral particle), including a particle containing an exogenous agent for a target cell. In some embodiments, the method of delivery includes administering a mobilization agent, e.g., a mobilization regimen, to the subject and administering the particle (e.g., lentiviral particle) to the subject in accordance with the provided methods. In some embodiments, the mobilization agent is administered to the subject prior to introducing or administering the particles (e.g., lentiviral particle) to the subject. In some embodiments, the mobilization agent includes a mobilization regimen that cause therapeutically inaccessible hematopoietic cells to become therapeutically accessible. In some embodiments, a mobilization agent increases the number of hematopoietic cells in peripheral blood, thus allowing for a more accessible source of hematopoietic cells for targeting by the particles (e.g., lentiviral particles) in accordance with the methods as described. In some embodiments, the mobilization agent stimulates mobilization of bone marrow cells from the bone marrow to the peripheral blood in the subject.

Also provided herein in some embodiments are methods involving administering to a subject (i) an agent that stimulates mobilization of bone marrow cells from the bone marrow to the peripheral blood in the subject and (ii) a particle, such as any described in Section III, either with or without any of the targeting agents described herein.

In some embodiments, the mobilization agent increases the number of stem cells in peripheral blood, thus allowing for a more accessible source of stem cells for use in the method as described. In some embodiments, the mobilization agent increases the number of hematopoietic cells in circulation in the subject. In some embodiments, the mobilization agent is a mobilizer of hematopoietic stem cells or progenitor cells. In some embodiments, the mobilization agent induces the exit of hematopoietic cells from bone marrow.

In some embodiments, the hematopoietic cells are CD34+ and may include CD34+ progenitor cells. In some embodiments, the hematopoietic cells are HSCs. In some aspects, the mobilization agent is a stem cell mobilization agent.

As used herein, “mobilizing” and “mobilizing hematopoietic cells” are used interchangeably to refer to the act of inducing the migration of hematopoietic cells, such as CD34+ cells, including progenitor cells and/or hematopoietic stem cells, from a first location (e.g., stem cell niche, e.g., bone marrow) into a second location (e.g., tissue (e.g., peripheral blood) or organ (e.g., spleen). In some embodiments, the process of mobilizing hematopoietic cells involves the recruitment of stem cells from their tissue or organ of residence to peripheral blood following treatment with a mobilization agent, such as using a mobilization agent known to a skilled artisan, including any cytokine and chemotherapeutic drugs known in the art for this purpose (e.g., G-CSF). In some aspects, this process mimics the enhancement of the physiological release of stem cells from tissues or organs in response to stress signals during injury and inflammation. In some embodiments, the mobilization agent or agents act as agonists or antagonists that prevent the attachment of hematopoietic cells to cells or tissues of their microenvironment. In some embodiments, the mobilization agent or agents induce the release of proteases that cleave the adhesion molecules or support structures between hematopoietic cells and their sites of attachment. In some embodiments, the mobilization agent is capable of mobilizing any hematopoietic cell, such as stem cells and/or progenitor cells, in which heparan sulfate proteoglycans are responsible for maintaining adhesion of the cells in their cell niche. In an aspect, a method of mobilizing hematopoietic cells in a subject comprises administering to a subject an effective amount of an agent that inhibits the level or activity of heparan sulfate proteoglycans, thereby mobilizing hematopoietic cells in the subject.

In some embodiments, a mobilization agent increases the circulation of hematopoietic cells and/or mobilizes hematopoietic cells sequestered in bone marrow to exit bone marrow into compartments where they are accessible, e.g., accessible for transduction by the lipid particle and/or viral vector. For example, administration to a subject of a mobilization therapy can increase the circulation of hematopoietic cells and/or mobilize hematopoietic cells sequestered in bone marrow to exit bone marrow into compartments where they are accessible, such as the peripheral blood.

In some embodiments, the mobilization agent is administered prior to the particle (e.g., lentiviral particle or other viral particle). In some embodiments, the mobilization agent is administered to the subject within 7 days prior to administering the particle (e.g., lentiviral particle), such as within 6 days, 5 days, 4 days, 3 days, 2 days or 1 day prior to administering the particle (e.g., lentiviral particle). In some embodiments, the mobilization agent is administered twice a day, once a day or two or three times within a week. In some embodiments, the mobilization agent is administered once daily for consecutive days prior to administering the particle (e.g., lentiviral particle). In some embodiments, at least one dose of the mobilization agent is administered to the subject on the same day as the provided methods of contacting target cells with a particle (e.g., lentiviral particle). In some embodiments, at least one dose of the mobilization agent is administered to the subject within 12 hours prior to administering the lipid particle (e.g. lentiviral vector), such as within 10 hours, 8 hours, 6 hours, 5 hours, 4 hours, 3 hours, 2 hours or 1 hour prior to administering the particle (e.g., lentiviral particle).

Exemplary mobilization agents include stem cell factor (SCF), small molecule VLA-4 inhibitor BI05192, BOP (N-(benzenesulfonyl)-L-prolyl-L-O-(1-pyrrolidinylcarbonyl)tyrosine), heparin, granulocyte colony-stimulating factor (G-CSF), and plerixafor (also known as AMD3100). In some embodiments, the mobilization agent of the method is selected from the group consisting of granulocyte-macrophage colony-stimulating factor (GM-CSF), Fms-related tyrosine kinase 3 (flt-3) ligand, stromal cell-derived factor 1 (SDF-1), agonists of the chemokine (C-C motif) receptor 1 (CCR1), such as chemokine (C-C motif) ligand 3 (CCL3, also known as macrophage inflammatory protein-1α (Mip-1α)), agonists of the chemokine (C-X-C motif) receptor 1 (CXCR1) and CXCR2, such as chemokine (C-X-C motif) ligand (CXCL1), CXCL2 (also known as growth-related oncogene protein-β (Gro-β)), and CXCL8 (also known as interleukin-8 (IL-8)), agonists of CXCR4, such as CTCE-002, ATI-2341, and Met-SDF-1, Very Late Antigen (VLA)-4 inhibitor, TG-0054, AMD3465, and any combination thereof.

In some embodiments, the mobilization agent is stem cell factor (SCF), small molecule VLA-4 inhibitor BI05192, BOP (N-(benzenesulfonyl)-L-prolyl-L-0-(1-pyrrolidinylcarbonyl)tyrosine), heparin, granulocyte colony-stimulating factor (G-CSF), or plerixafor (AMD3100). In some embodiments, the mobilization agent includes the combination of G-CSF and plerixafor (AMD3100). In some of any embodiments, G-CSF is filgrastim (e.g. Neupogen® or Zarzio®). In some embodiments, the G-CSF is a pegylated G-CSF, such as pegfilgrastim (e.g. Neulasta®).

Any of various known methods for mobilizing hematopoietic cells using a mobilization agent can be used, including but not limited to, any as described in PCT publication No. WO2021211450; U.S. publication Nos. US20200268850 and US20170106021; and U.S. Pat. Nos. 7,939,057 and 10,907,177.

In some embodiments, the mobilization regimen includes administration of at least one mobilizing agent. In various embodiments, at least one mobilizing agent is administered to a subject (i) daily on the four days prior to administration of a first dose of the particle (e.g., lentiviral particle); (ii) on the day of administration of a first dose of the particle (e.g., lentiviral particle); and (iii) on the day of administration of one or more subsequent doses of the particle (e.g., lentiviral particle). In some embodiments, at least one mobilizing agent is administered to a subject (i) on the day prior to administration of a first dose of the particle (e.g., lentiviral particle) and (ii) on the day of administration of a first dose of the particle (e.g., lentiviral particle).

In some embodiments, the mobilization regimen includes administration of one or both of G-CSF and plerixafor/AMD3100. In various embodiments G-CSF is administered to a subject (i) daily on the four days prior to administration of a first dose of the particle (e.g., lentiviral particle); (ii) on the day of administration of a first dose of the particle (e.g., lentiviral particle); and (iii) on the day of administration of one or more subsequent doses of the particle (e.g., lentiviral particle). In various embodiments plerixafor/AMD3100 is administered to a subject (i) on the day prior to administration of a first dose of the particle (e.g., lentiviral particle) and (ii) on the day of administration of a first dose of the particle (e.g., lentiviral particle).

In some embodiments, the at least one mobilizing agent is administered once daily at a dose that is, or is at least, 0.1, 1.0, 10, 20, 30, 40, 50, 75, 100, 150, or 200 μg/kg. In various embodiments, a daily dose of the at least one mobilizing agent has a range having a lower bound of 0.1 μg/kg/day, 1.0 μg/kg/day, 10 μg/kg/day, 20 μg/kg/day, 30 μg/kg/day, 40 μg/kg/day, 50 μg/kg/day, or 75 μg/kg/day and an upper bound of 100 μg/kg/day, 150 μg/kg/day, or 200 μg/kg/day. In various embodiments the at least one mobilizing agent is administered once daily at a dose that is, or is at least, 1 mg/kg, 2 mg/kg, 3 mg/kg, 4 mg/kg, 5 mg/kg, 7.5 mg/kg, 10 mg/kg, 15 mg/kg, or 20 mg/kg. In various embodiments, a daily dose of the at least one stem cell mobilizing agent has a range having a lower bound of 1 mg/kg/day, 2 mg/kg/day, 3 mg/kg/day, 4 mg/kg/day, 5 mg/kg/day, or 7.5 mg/kg/day and an upper bound of 10 mg/kg/day, 15 mg/kg/day, or 20 mg/kg/day.

In various embodiments G-CSF is administered once daily at a dose that is, or is at least, 10 μg/kg, 20 μg/kg, 30 μg/kg, 40 μg/kg, 50 μg/kg, 75 μg/kg, 100 μg/kg, 150 μg/kg, or 200 μg/kg. In various embodiments, a daily dose of G-CSF has a range having a lower bound of 10 μg/kg/day, 20 μg/kg/day, 30 μg/kg/day, 40 μg/kg/day, 50 μg/kg/day, or 75 μg/kg/day and an upper bound of 100 μg/kg/day, 150 μg/kg/day, or 200 μg/kg/day. In various embodiments plerixafor/AMD3100 is administered once daily at a dose that is, or is at least, 1 mg/kg, 2 mg/kg, 3 mg/kg, 4 mg/kg, 5 mg/kg, 7.5 mg/kg, 10 mg/kg, 15 mg/kg, or 20 mg/kg. In various embodiments, a daily dose of G-CSF has a range having a lower bound of 1 mg/kg/day, 2 mg/kg/day, 3 mg/kg/day, 4 mg/kg/day, 5 mg/kg/day, or 7.5 mg/kg/day and an upper bound of 10 mg/kg/day, 15 mg/kg/day, or 20 mg/kg/day.

In some embodiments, G-CSF may be administered daily as a dose of 0.5-16 μg/kg (e.g., 5-16 μg/kg or 10-16 μg/kg) for 1-10 days (e.g., 1-7 days, or particularly, 1-3 days). In another example, G-CSF may be administered to a healthy donor at a dosage of 10-16 μg/kg daily for up to seven days. Three or four days of treatment may be sufficient when the peripheral blood collections are combined with apheresis starting on, e.g., day 4. In another example, G-CSF may be administered at a dosage of 10 μg/kg daily for four days with apheresis starting on, e.g., day 4. The most commonly used dosage of G-CSF in healthy donors is 10 μg/kg body weight daily with leukapheresis starting on day 5 onward until collection of an adequate number of stem cells (e.g., collections can be taken once or twice daily for 1 to 4 days, such as 1 or 2 days). G-CSF may be administered subcutaneously or intravenously. Methods of G-CSF administration and dosage are described in Juttner et al. (Blood 89:2233-2258, 1997), Kroschinsky et al. (Haematologica 90:1556-1671, 2005), U.S. Pat. No. 6,162,427, 2005/0186182, WO 2010051335, and WO 2005014023, all of which are incorporated herein by reference in their entireties. If cyclophosphamide is administered for mobilization, G-CSF is usually started 2-5 days after completion of cyclophosphamide infusion. Methods of administering combined mobilization agents of G-CSF and one or more chemotherapeutic agents are described in Andre et al. (Transfusion 43:50-57, 2003), Ataergin et al. (Am. J. Hematol. 83:644-648, 2008), and Demirer et al. (Br. J. Haematol. 116:468-474, 2002), all of which are incorporated herein by reference in their entireties.

Plerixafor may be administered at a dosage of 1-300 μg/kg. At 240 μg/kg, the number of mobilized stem cells peak at around 4-10 hours after plerixafor administration. In some examples, plerixafor may be administered once or twice daily at a dose of ^{~}1-300 μg/kg (e.g., 100-300 μg/kg or 200-300 μg/kg) for 1-10 days (e.g., 1-5 days).

The combination of plerixafor and G-CSF for stem cell mobilization was approved by the FDA in 2008 for use in patients with non-Hodgkin's lymphoma and multiple myeloma. This combination therapy may also be used to mobilize stem cells. A typical combination therapy may include, e.g., the administration of G-CSF at ^{~}0.5-16 g/kg (e.g., 10 g/kg) daily with plerixafor at 1-300 g/kg (e.g., 240 g/kg) given a few days (e.g., 1-3 days) after G-CSF administration. Both agents may be given together for about 2-10 days (e.g., 4 days) or until adequate hematopoietic cells are collected. Plerixafor, either alone or in combination with G-CSF, may be administered subcutaneously or intravenously.

In some embodiments, the method of mobilizing hematopoietic cells in a subject includes administering to the subject at least one mobilization agent. In some embodiments, the method of mobilizing hematopoietic cells includes administering to the subject at least one mobilization agent comprising (i) at least one heparan sulfate inhibitor and (ii) at least one of a CXCR2 agonist and a CXCR4 antagonist.

In some embodiments, the mobilized hematopoietic cells comprise KLS-CD150+CD48− cells. In some embodiments, the mobilized hematopoietic cells comprise CD34− CD133+ cells. In some embodiments, the mobilized hematopoietic cells and/or progenitor cells comprise common myeloid progenitor cells. In some embodiments, the mobilized hematopoietic cells and/or progenitor cells comprise granulocyte/monocyte progenitor cells. In some embodiments, the mobilized hematopoietic cells and/or progenitor cells comprise megakaryocyte/erythroid progenitor cells. In some embodiments, the mobilized hematopoietic cells and/or progenitor cells comprise committed lymphoid progenitor cells. In some embodiments, the mobilized hematopoietic cells and/or progenitor cells comprise a combination of common myeloid progenitor cells, granulocyte/monocyte progenitor cells, megakaryocyte/erythroid progenitor cells. In some embodiments, the hematopoietic progenitor cells comprise CD150− CD48− CD244+ cells. In some embodiments, the hematopoietic progenitor cells comprise CD150− CD48+CD244+ cells. In some embodiments, the hematopoietic progenitor cells comprise Lin-SCA-1-c-Kit+CD34+CD16/32mid cells. In some embodiments, the hematopoietic progenitor cells comprise lin-SCA-1-c-kit+CD34− CD16/32low cells. In some embodiments, the mobilized hematopoietic cells comprise CD34+ peripheral blood stem cells.

### VII. EXEMPLARY EMBODIMENTS

Among the provided embodiments are:

1. A lipid particle, comprising:
(a) a retargeted attachment protein comprising (i) a paramyxovirus envelope attachment protein; and (ii) a targeting moiety directed to a target molecule expressed on the surface of a target cell,
(b) a co-display protein; and
(c) at least one paramyxovirus fusion (F) protein; and
wherein the co-display protein increases the potency and/or specificity of the lipid particle as compared to a lipid particle not comprising the co-display protein,
wherein the protein in (a), (b) and (c) are exposed on the outside of the lipid bilayer/embedded in the lipid bilayer.

2. A lipid particle, comprising:
(a) a retargeted attachment protein comprising (i) a paramyxovirus envelope attachment protein; and (ii) a targeting moiety directed to a target molecule expressed on the surface of a target cell,
(b) a co-display protein for increasing the potency and/or specificity of the lipid particle as compared to a lipid particle not comprising the co-display protein; and
(c) at least one paramyxovirus fusion (F) protein; and
wherein the protein in (a), (b) and (c) are exposed on the outside of the lipid bilayer/embedded in the lipid bilayer.

3. A lipid particle, comprising:
(a) a retargeted attachment protein comprising (i) a paramyxovirus envelope attachment protein; and (ii) a targeting moiety directed to a first target molecule expressed on the surface of a target cell,
(b) a co-display protein comprising a binding means for specifically interacting with a second target molecule expressed on the surface of the target cell; and
(c) at least one paramyxovirus fusion (F) protein; and
wherein the protein in (a), (b) and (c) are exposed on the outside of the lipid bilayer/embedded in the lipid bilayer.

4. A lipid particle, comprising:
(a) a Baboon Endogenous Virus (BaEV) fusogen or functional variant thereof, and
(b) a co-display protein comprising a binding means for specifically interacting with a target molecule expressed on the surface of the target cell; and
wherein the protein in (a) and (b) are exposed on the outside of the lipid bilayer/embedded in the lipid bilayer.

5. The lipid particle of any of embodiments 1-4, wherein the co-display protein is an immune stimulating protein and/or is a transduction adjuvant.

6. A lipid particle, comprising:
(a) a retargeted attachment protein comprising (i) a paramyxovirus envelope attachment protein; and (ii) a targeting moiety directed to a target molecule expressed on the surface of a target cell,
(b) a co-display protein selected from an immune stimulating protein and a transduction adjuvant; and
(c) at least one paramyxovirus fusion (F) protein; and wherein the protein in (a), (b) and (c) are embedded in the lipid bilayer.

7. The lipid particle of embodiments 1-6, wherein the co-display protein is selected from the group comprising of a glycoprotein, transmembrane protein, receptor, integrin, antibody or antigen-binding fragment, an engineered binding domain, a nanobody, a DARPin, an Aptamer, an Affimer, an Affibody, a Knottin, an Avimer, a Monobody, an Anticalin, a Fynomer, and a targeting peptide.

8. The lipid particle of any of embodiments 1-7, wherein the co-display protein increases specificity to the target cell.

9. The lipid particle of any of embodiments 1-8, wherein the co-display protein increases specificity to a target cell selected from the group consisting of tumor-infiltrating lymphocytes, T cells, neoplastic or tumor cells, virus-infected cells, stem cells, central nervous system (CNS) cells, hematopoietic stem cells (HSCs), and liver cells.

10. The lipid particle of any of embodiments 1-9, wherein the co-display protein increases specificity to a target cell selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

11. The lipid particle of any of embodiments 1-10, wherein the co-display protein increases specificity to a target cell that is a hematopoietic stem cell.

12. The lipid particle of embodiment 11, wherein the cell surface molecule is selected from CD34, CD105, or CD117.

13. The lipid particle of any of embodiments 1-10, wherein the co-display protein increases specificity to a target cell that is a T cell.

14. The lipid particle of embodiment 13, wherein the cell surface molecule is selected from CD3, CD4, CD7 CD8, ASCT2, CD110, CD133, CD146, CD164, CD46, CD49f, CD90, EPCR, and ITGA3.

15. The lipid particle of any of embodiments 1-10, wherein the co-display protein increases specificity to a target cell that is a hepatocyte.

16. The lipid particle of embodiment 15, wherein the cell surface molecule is selected from ASGR1, ASGR2 and TM4SF5.

17. The lipid particle of any of embodiments 1-3, 5-16, wherein the co-display protein binds to a different protein than the targeting moiety of the retargeted attachment protein.

18. The lipid particle of embodiment 1, wherein the co-display protein does not include a paramyxovirus envelope attachment protein or a fragment thereof.

19. The lipid particle of any of embodiments 1-10, 13, 14, 17 or 18, wherein the co-display protein is an immune stimulating protein and the immune stimulating protein stimulates a T cell activity or function.

20. The lipid particle of embodiment 19, wherein the immune stimulating protein comprises
(i) an extracellular domain,
(ii) a linker, and
(iii) an anchor.

21. The lipid particle of embodiment 20, wherein the extracellular domain of the immune stimulating protein binds to a T cell stimulatory receptor.

22. The lipid particle of embodiment 21, wherein the T cell stimulatory receptor is selected from CD3, CD2, CD28, CD27.

23. The lipid particle of embodiments 20-22, wherein the extracellular domain of the immune stimulating protein binds to CD3.

24. The lipid particle of embodiments 20-23, wherein the extracellular domain is selected from the group comprising an antibody or antigen-binding fragment, an engineered binding domain, a nanobody, a DARPin, an Aptamer, an Affimer, an Affibody, a Knottin, an Avimer, a Monobody, an Anticalin, a Fynomer, and a targeting peptide.

25. The lipid particle of embodiments 20-24, wherein the extracellular domain of the immune stimulating protein is an anti-CD3 antibody or single chain fragment thereof.

26. The lipid particle of any of embodiments 1-18, wherein the co-display protein is a transduction adjuvant that increases transduction efficiency of the lipid particle as compared to a lipid particle that does not have a co-display protein comprising a transduction adjuvant.

27. The lipid particle of any of embodiments 1-18, and 26, wherein the co-display protein is a transduction adjuvant that binds to a surface molecule on target cells to enhance transduction.

28. The lipid particle of embodiment 26 or 27 wherein the transduction adjuvant comprises
(i) an extracellular domain,
(ii) a linker, and
(iii) an anchor.

29. The lipid particle of embodiment 28, wherein the extracellular domain of the transduction adjuvant binds an integrin binding domain.

30. The lipid particle of embodiments 28 or 29, wherein the extracellular domain of the transduction adjuvant comprises the extracellular domain of fibronectin, retronectin, or a functional variant thereof.

31. The lipid particle of any of embodiments 20-30, wherein the linker is derived from a human protein.

32. The lipid particle of any of embodiments 20-31, wherein the linker is a derived from human CD8α, IgG3 hinge, IgG1 hinge, IgG4 heavy chain, IgG4, or CD28.

33. The lipid particle of any of embodiments 20-32, wherein the linker is modified to remove a cysteine residue.

34. The lipid particle of any of embodiments 20-33, wherein the linker is rigid or wherein the linker is flexible.

35. The lipid particle of any of embodiments 20-34, wherein the anchor is a peptide anchor that is a transmembrane domain.

36. The lipid particle of any of embodiments 20-35, wherein the anchor is a transmembrane domain that is a transmembrane domain of a protein selected from PDGFR, VSV-G, CD8α, ICAM1, HLA-DRB, CD49d, or Transferrin.

37. The lipid particle of any of embodiments 20-34, wherein the anchor is a GPI lipid anchor.

38. The lipid particle of any of embodiments 1-3, 5-37, wherein the targeting moiety is selected from the group consisting of an antibody or antigen-binding fragment, an engineered binding domain, a nanobody, a DARPin, an Aptamer, an Affimer, an Affibody, a Knottin, an Avimer, a Monobody, an Anticalin, a Fynomer, and a targeting peptide.

39. The lipid particle of any of embodiments 1-3, 5-38, wherein the targeting moiety binds to a cell surface molecule present on a target cell.

40. The lipid particle of embodiment 39, wherein the cell surface molecule is a protein, glycan, or lipid.

41. The lipid particle of any of embodiments 1-3, 5-40, wherein the target cell is selected from the group consisting of tumor-infiltrating lymphocytes, T cells, neoplastic or tumor cells, virus-infected cells, stem cells, central nervous system (CNS) cells, hematopoietic stem cells (HSCs), and liver cells.

42. The lipid particle of any of embodiments 1-3, 5-41, wherein the target cells is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

43. The lipid particle of any of embodiments 1-3, 5-42, wherein the target is a hematopoietic stem cell.

44. The lipid particle of any of embodiment 43, wherein the cell surface molecule is selected from CD34, CD105, or CD117.

45. The lipid particle of any of embodiments 1-3, 5-42, wherein the target cell is a T cell.

46. The lipid particle of embodiment 45, wherein the cell surface molecule is selected from the group consisting of CD3, CD4, CD7 CD8, ASCT2, CD110, CD133, CD146, CD164, CD46, CD49f, CD90, EPCR, and ITGA3.

47. The lipid particle of any of embodiments 1-3, 5-42, wherein the target cell is a hepatocyte.

48. The lipid particle of embodiment 47, wherein the cell surface molecule is selected from the group consisting of ASGR1, ASGR2 and TM4SF5.

49. The lipid particle of any of embodiments 1-3, 5-48, wherein the targeting moiety is selected from the group consisting of a single domain antibody or a single chain variable fragment (scFv).

50. The lipid particle of embodiment 49, wherein the single domain antibody is a VHH.

51. The lipid particle of any of embodiments 1-3, and 5-50, wherein the retargeted attachment protein is linked to a (i) first targeting moiety directed to a first target molecule expressed on the surface of a target cell, and a (ii) second targeting moiety directed to a second target molecule expressed on the surface of a target cell.

52. The lipid particle of any of embodiments 1-3, 5-51, wherein the paramyxovirus envelope attachment protein of the retargeted attachment protein is a first paramyxovirus envelope attachment protein and the lipid particle further comprises a second paramyxovirus envelope attachment protein.

53. The lipid particle of any of embodiments 1-3, 5-52, wherein the second paramyxovirus envelope attachment protein is linked to the first paramyxovirus envelope attachment protein in tandem.

54. The lipid particle of any of embodiments 1-3, 5-53, wherein the retargeted attachment protein comprising the first paramyxovirus envelope attachment protein and the second paramyxovirus envelope attachment protein are each separately exposed the outside of the lipid bilayer/embedded in the lipid bilayer.

55. The lipid particle of any of embodiments 1-3, 5-54, wherein the second paramyxovirus attachment protein is a retargeted attachment protein.

56. The lipid particle of embodiment 55, wherein the second retargeted attachment protein comprises (i) a second paramyxovirus envelope attachment protein; and (ii) a second targeting moiety directed to a target molecule expressed on the surface of a target cell.

57. The lipid particle of any of embodiments 51-56, wherein the targeting moiety is selected from the group consisting of an antibody or antigen-binding fragment, an engineered binding domain, a nanobody, a DARPin, an Aptamer, an Affimer, an Affibody, a Knottin, an Avimer, a Monobody, an Anticalin, a Fynomer, and a targeting peptide.

58. The lipid particle of embodiment 56 or 57, wherein the target cell is selected from the group consisting of tumor-infiltrating lymphocytes, T cells, neoplastic or tumor cells, virus-infected cells, stem cells, central nervous system (CNS) cells, hematopoietic stem cells (HSCs), and liver cells.

59. The lipid particle of any of embodiments 56-58, wherein the target cells is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

60. The lipid particle of any of embodiments 56-59, wherein the target is a hematopoietic stem cell.

61. The lipid particle of any of embodiment 60, wherein the cell surface molecule is selected from CD34, CD105, or CD117.

62. The lipid particle of any of embodiments 56-60, wherein the target cell is a T cell.

63. The lipid particle of embodiment 62, wherein the cell surface molecule is selected from the group consisting of CD3, CD4, CD7 CD8, ASCT2, CD110, CD133, CD146, CD164, CD46, CD49f, CD90, EPCR, and ITGA3.

64. The lipid particle of any of embodiments 56-60, wherein the target cell is a hepatocyte.

65. The lipid particle of embodiment 64, wherein the cell surface molecule is selected from the group consisting of ASGR1, ASGR2 and TM4SF5.

66. The lipid particle of any of embodiments 56-65, wherein the targeting moiety is selected from the group consisting of a single domain antibody or a single chain variable fragment (scFv).

67. The lipid particle of embodiment 66, wherein the single domain antibody is a VHH.

68. The lipid particle of embodiment 4, wherein the BaEV envelope glycoprotein is a truncated BaEV envelope glycoprotein comprising a cytoplasmic tail with a partial fusion inhibitory R peptide relative to the inhibitory R peptide of a wild-type BaEV envelope glycoprotein, wherein the partial fusion inhibitory R peptide comprises at least one amino-terminal amino acid but less than the full length of the inhibitory R peptide of the wild-type BaEV envelope glycoprotein.

69. The lipid particle of embodiment 4 or 68, wherein the BaEV envelope glycoprotein is a truncated BaEV envelope glycoprotein comprising a cytoplasmic tail with a partial fusion inhibitory R peptide relative to a wild-type BaEV envelope glycoprotein, wherein the cytoplasmic tail is 25 amino acids in length and contains 8 contiguous amino-terminal acids of the inhibitory R peptide (R+8) of the full length inhibitory R peptide of wild-type BaEV envelope glycoprotein.

70. The lipid particle of embodiments 68 or 69, wherein the truncated BaEV glycoprotein comprises: (i) a glycoprotein 70 (g70) subunit or a biologically active portion thereof, and (ii) a portion of the glycoprotein p20E (p20E) subunit comprising the cytoplasmic tail with the partial inhibitory R peptide.

71. The lipid particle of embodiments 68-70, wherein the BaEV envelope glycoprotein binds an ASCT-2 or ASCT-1 receptor.

72. The lipid particle of any of embodiments 68-71, wherein the truncated BaEV envelope glycoprotein is truncated relative to SEQ ID NO: 622 and lacks up to 16 contiguous amino acids from the C-terminal cytoplasmic tail of SEQ ID NO: 622.

73. The lipid particle of any of embodiments 68-72, wherein the partial fusion inhibitory R peptide is set forth as amino acids 1 to 8 of SEQ ID NO: 623, optionally wherein the cytoplasmic tail is set forth in SEQ ID NO: 624 (R+8).

74. The lipid particle of any of embodiments 68-73, wherein the truncated BaEV envelope glycoprotein is set forth in SEQ ID NO: 625.

75. A lipid particle, comprising a variant Baboon Endogenous Virus (BaEV) fusogen wherein the fusogen is exposed on the outside of the lipid bilayer/embedded in the lipid bilayer.

76. The lipid particle of embodiment 75, wherein the variant BaEV envelope glycoprotein has the amino acid sequence set forth in SEQ ID NO: 795 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:795.

77. The lipid particle of embodiment 75, wherein the variant BaEV envelope glycoprotein has the amino acid sequence set forth in SEQ ID NO: 796 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:796.

78. The lipid particle of embodiment 75, wherein the variant BaEV envelope glycoprotein has the amino acid sequence set forth in SEQ ID NO: 797 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:797.

79. The lipid particle lipid particle of embodiment 75, wherein the BaEV Baboon Endogenous Virus (BaEV) is a BaEVTR fusogen.

80. The lipid particle lipid particle of embodiment 79, wherein the BaEV Baboon Endogenous Virus (BaEV) fusogen or functional variant thereof comprises a protease site modification.

81. The lipid particle of embodiment 80, wherein the protease site modification comprises modifying a MLV-A cleavage site to a MA/CA cleavage site.

82. The lipid particle of any of embodiments 1-3, 5-67, wherein the paramyxovirus envelope attachment protein is an envelope attachment protein from a Nipah virus, Hendra virus, or Measles virus, or is a variant or biologically active portion thereof of any of the foregoing.

83. The lipid particle of any of embodiments 1-3, 5-67, or 82, wherein the paramyxovirus envelope attachment protein is a wild-type paramyxovirus G protein, H protein or HN protein or is a variant or biologically active portion of any of the foregoing.

84. The lipid particle of embodiment 82 or embodiment 83 wherein the paramyxovirus envelope attachment protein is a wild-type Nipah virus G (NiV-G) protein or is a variant or biologically active portion of a NiV-G.

85. The lipid particle of embodiments 1-3, 5-67, or 82-84, wherein the paramyxovirus envelope attachment protein is a variant paramyxovirus envelope attachment protein.

86. The lipid particle of embodiment 85, wherein the variant paramyxovirus envelope attachment protein comprises one or more mutations that reduces native tropism relative to the wild-type paramyxovirus envelope attachment protein not comprising the one or more mutations.

87. The lipid particle of any of embodiments 1-3, 5-67, or 82-86, wherein the paramyxovirus envelope attachment protein is a variant NiV-G that is a variant or a biologically active portion of a wild-type NiV-G.

88. The lipid particle of any one of embodiments 85-87, wherein the variant is a variant NiV-G that is a variant of a wild-type Nipah virus G (NiV-G) protein or a biologically active portion thereof.

89. The lipid particle of embodiment 87 or 88, wherein the variant NiV-G is truncated by up to 40 contiguous amino acids at or near the N-terminus of the wild-type NiV-G set forth in SEQ ID NO:1.

90. The lipid particle of any of embodiments 87-89, wherein the variant NiV-G has a truncation of amino acids 2-34 of the wild-type NiV-G set forth in SEQ ID NO:1.

91. The lipid particle of any of embodiments 87-90, wherein the variant NiV-G exhibits reduced binding to Ephrin B2 or Ephrin B3.

92. The lipid particle of embodiment 91, wherein the variant NiV-G comprises: one or more amino acid substitutions corresponding to amino acid substitutions selected from the group consisting of E501A, W504A, Q530A and E533A with reference to numbering set forth in SEQ ID NO:1.

93. The lipid particle of embodiment 91 or embodiment 92, wherein the variant NiV-G comprises amino acid substitutions E501A, W504A, Q530A and E533A with reference to numbering set forth in SEQ ID NO:1.

94. The lipid particle of any of embodiments 87-93, wherein the variant NiV-G has the amino acid sequence set forth in SEQ ID NO: 228 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:228.

95. The lipid particle of any of embodiments 87-94, wherein the variant NiV-G has the amino acid sequence set forth in SEQ ID NO:228.

96. The lipid particle of any of embodiments 1-95, wherein the particle further comprises an additional attachment protein that is a paramyxovirus envelope attachment protein that is a variant NiV-G that is a variant or a biologically active portion of a wild-type NiV-G.

97. The lipid particle of embodiment 96, wherein the variant NiV-G exhibits reduced binding to Ephrin B2 or Ephrin B3.

98. The lipid particle of embodiment 97, wherein the variant NiV-G comprises: one or more amino acid substitutions corresponding to amino acid substitutions selected from the group consisting of E501A, W504A, Q530A and E533A with reference to numbering set forth in SEQ ID NO:1.

99. The lipid particle of any of embodiments 1-3, 5-67, 82-98, wherein the at least one paramyxovirus fusion (F) protein is an F protein from a henipavirus or is a biologically active portion thereof or variant thereof.

100. The lipid particle of embodiment 99, wherein the henipavirus is a Hendra virus.

101. The lipid particle of embodiment 99, wherein the henipavirus is a Nipah virus.

102. The lipid particle of any of embodiments 1-3, 5-67, 82-98, wherein the paramyxovirus F protein is a wild-type NiV-F protein or a variant or a biologically active portion thereof.

103. The lipid particle of any of embodiments 1-3, 5-67, 82-98, wherein the paramyxovirus F protein is a variant NiV-F that is a variant or a biologically active portion of a wild-type NiV-F protein.

104. The lipid particle of embodiment 103, wherein the variant NiV-F is truncated by up to 22 contiguous amino acids the at the C-terminus of the wild-type NiV-F set forth in SEQ ID NO:235, optionally not include the initial methionine.

105. The lipid particle of embodiment 103 or embodiment 104, wherein the variant NiV-F protein is a truncated NiV-F that lacks amino acids 525-546 of SEQ ID NO:235.

106. The lipid particle of any of embodiments 103-105, wherein the variant NiV-F has the amino acid sequence set forth in SEQ ID NO: 227 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:227.

107. The lipid particle of any of embodiments 103-106, wherein the variant NiV-F has the amino acid sequence set forth in SEQ ID NO:227.

108. The lipid particle of any of embodiments 1-3, 5-67, 82-103, wherein the paramyxovirus F protein is an F0 precursor or is a proteolytically cleaved form thereof comprising F1 and F2 subunits.

109. The lipid particle of embodiment 108, wherein the proteolytically cleaved form is a cathepsin L cleavage product.

110. The lipid particle of any of embodiments 1-109, wherein the targeting moiety and the paramyxovirus envelope attachment protein or biologically active portion thereof is attached via a linker.

111. The lipid particle of embodiment 110, wherein the linker is a peptide linker.

112. The lipid particle of embodiment 111, wherein the peptide linker is 2 to 65 amino acids in length.

113. The lipid particle of embodiment 111 or embodiment 112, wherein the peptide linker is a flexible linker that comprises GS, GGS, GGGGS, GGGGGS or combinations thereof.

114. The lipid particle of any of embodiments 111-113, wherein the peptide linker is selected from: (GGS)n, wherein n is 1 to 10; (GGGGS)n, wherein n is 1 to 10; or (GGGGGS)n, wherein n is 1 to 6.

115. The lipid particle of any of embodiments 1-114, wherein the paramyxovirus fusion (F) protein exhibits fusogenic activity with a target cell upon binding of at least one paramyxovirus envelope attachment protein o to the target molecule on the target cell.

116. The lipid particle of any of embodiments 1-115, wherein the lipid particle comprises a viral nucleic acid.

117. The lipid particle of embodiment 116, wherein the viral nucleic acid comprises one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

118. The lipid particle of any of embodiments 1-117, wherein the lipid particle is a viral vector. 119. The lipid particle of any of embodiments 1-117, that is a retroviral vector.

120. The lipid particle of any of embodiments 1-119, that is a lentiviral vector.

121. The lipid particle of any of embodiments 1-117, wherein the lipid particle is devoid of viral genomic DNA.

122. The lipid particle of any of embodiments 1-117 and 121, that is a viral-like particle.

123. The lipid particle of any of embodiments 1-117, 121, and 122, that is a retroviral-like particle.

124. The lipid particle of any of embodiments 1-117, 121, and 122, that is a lentiviral-like particle.

125. The lipid particle of embodiment 120 or 124, wherein the lentiviral vector or the lentiviral-like particle is derived from HIV.

126. The lipid particle lipid particle of embodiment 4 and 125, wherein the Baboon Endogenous Virus (BaEV) is a BaEVTR fusogen.

127. The lipid particle lipid particle of embodiment 4 and 125, wherein the Baboon Endogenous Virus (BaEV) fusogen or functional variant thereof comprises a protease site modification.

128. The lipid particle of embodiment 127, wherein the protease site modification comprises modifying a MLV-A cleavage site to a MA/CA cleavage site.

129. The lipid particle of any of embodiments 1-128, wherein the lipid particle is produced as a preparation with increased titer compared to a reference lipid particle preparation that is similarly produced but with only the first retargeted attachment protein.

130. The lipid particle of embodiment 129, wherein the titer is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.

131. The lipid particle of any of embodiments 1-130, further comprising an exogenous agent for delivery to a target cell.

132. The lipid particle of embodiment 131, wherein the exogenous agent is present in the lumen.

133. The lipid particle of embodiment 131 or embodiment 132, wherein the exogenous agent is a protein or a nucleic acid, optionally wherein the nucleic acid is a DNA or RNA.

134. The lipid particle of any of embodiments 131-133, wherein the exogenous agent is a nucleic acid encoding a cargo for delivery to the target cell.

135. The lipid particle of any of embodiments 131-134, wherein the exogenous agent is or encodes a therapeutic agent, a diagnostic agent or a genome-modifying enzyme.

136. The lipid particle of any of embodiments 131-135, wherein the exogenous agent encodes a membrane protein, optionally wherein the membrane protein is an antigen receptor for targeting cells expressed by or associated with a disease or condition.

137. The lipid particle of embodiment 136, wherein the membrane protein is a chimeric antigen receptor (CAR).

138. The lipid particle of any of embodiments 131-136, wherein the exogenous agent is a nucleic acid comprising a payload gene for correcting a genetic deficiency, optionally a genetic deficiency in the target cell, optionally wherein the genetic deficiency is associated with a liver cell or a hepatocyte.

139. The lipid particle of any of embodiments 131-138, wherein binding of the paramyxovirus envelope attachment protein or biologically active portion thereof to a target molecule expressed on the surface of a target cell mediates fusion of the particle with the target cell and delivery of the exogenous agent to the target cell.

140. The lipid particle of any of embodiments 131-139, wherein at or greater than 10%, 20%, 30%, 40%, 50%, 60% of the target cells are delivered the exogenous agent.

141. The lipid particle of any of embodiments 131-140, wherein delivery of the exogenous cell to the target cell is increased compared to a reference particle preparation that is similarly produced but with only a retargeted attachment protein (e.g., no co-display protein).

142. The lipid particle of embodiment 141, wherein the delivery to the target cell is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.

143. A producer cell comprising (a) a nucleic acid encoding a retargeted attachment protein comprising a paramyxovirus envelope attachment protein; and (i) a targeting moiety directed to a target molecule expressed on the surface of a target cell, (b) a nucleic acid encoding a co-display protein, and (c) a nucleic acid encoding at least one paramyxovirus F protein.

144. A producer cell comprising (a) a nucleic acid encoding a retargeted attachment protein comprising a Baboon Endogenous Virus (BaEV) fusogen or functional variant thereof expressed on the surface of a target cell, and (b) a nucleic acid encoding a co-display protein.

145. The producer cell of embodiment 143 or embodiment 144, wherein the cell further comprises a viral nucleic acid(s).

146. The producer cell of embodiment 145, wherein the viral nucleic acid(s) are lentiviral nucleic acids.

147. The producer cell of any of embodiments 143-146, wherein the cell is a mammalian cell.

148. The producer cell of any of embodiments 143-147, wherein the producer cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

149. The producer cell of any of embodiments 143-148, wherein the producer cell comprises 293T cells.

150. The producer cell of any of embodiments 143-149, wherein the viral nucleic acid(s) lacks one or more genes involved in viral replication.

151. The producer cell of any of embodiments 143-150, wherein the viral nucleic acid comprises a nucleic acid encoding a viral packaging protein selected from one or more of Gag, Pol, Rev and Tat.

152. The producer cell of any of embodiments 143-151, wherein the viral nucleic acid comprises: one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

153. The producer cell of any of embodiments 143-152, wherein the co-display protein increases specificity to the target cell.

154. The producer cell of any of embodiments 136-147, wherein the co-display protein increases specificity to a target cell selected from the group consisting of tumor-infiltrating lymphocytes, T cells, neoplastic or tumor cells, virus-infected cells, stem cells, central nervous system (CNS) cells, hematopoietic stem cells (HSCs), and liver cells.

155. The producer cell of any of embodiments 143-154, wherein the co-display protein increases specificity to a target cell selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

156. The producer cell of any of embodiments 143-155, wherein the co-display protein increases specificity to a target cell that is a hematopoietic stem cell.

157. The producer cell of embodiment 156, wherein the cell surface molecule is selected from CD34, CD105, or CD117.

158. The producer cell of any of embodiments 143-155, wherein the co-display protein increases specificity to a target cell that is a T cell.

159. The producer cell of embodiment 158, wherein the cell surface molecule is selected from CD3, CD4, CD7 CD8, ASCT2, CD110, CD133, CD146, CD164, CD46, CD49f, CD90, EPCR, and ITGA3.

160. The producer cell of any of embodiments 143-155, wherein the co-display protein increases specificity to a target cell that is a hepatocyte.

161. The producer cell of embodiment 160, wherein the cell surface molecule is selected from ASGR1, ASGR2 and TM4SF5.

162. The producer cell of any of embodiments 143, 145-161, wherein the co-display protein binds to a different protein than the targeting moiety of the retargeted attachment protein.

163. The producer cell of any of embodiments 143-162, wherein the co-display protein does not include a paramyxovirus envelope attachment protein or a fragment thereof.

164. The producer cell of any of embodiments 143-155, 158, 159, 162, or 163, wherein the co-display protein is an immune stimulating protein and the immune stimulating protein stimulates a T cell activity or function.

165. The producer cell of embodiment 164, wherein the immune stimulating protein comprises
(i) an extracellular domain,
(ii) a linker, and
(iii) an anchor.

166. The producer cell of embodiment 165, wherein the extracellular domain of the immune stimulating protein binds to a T cell stimulatory receptor.

167. The producer cell of embodiment 166, wherein the T cell stimulatory receptor is selected from CD3, CD2, CD28, CD27.

168. The producer cell of embodiments 165-167, wherein the extracellular domain of the immune stimulating protein binds to CD3.

169. The producer cell of embodiments 165-168, wherein the extracellular domain is selected from the group comprising an antibody or antigen-binding fragment, an engineered binding domain, a nanobody, a DARPin, an Aptamer, an Affimer, an Affibody, a Knottin, an Avimer, a Monobody, an Anticalin, a Fynomer, and a targeting peptide.

170. The producer cell of embodiments 165-169, wherein the extracellular domain of the immune stimulating protein is an anti-CD3 antibody or single chain fragment thereof.

171. The producer cell of any of embodiments 143-170, wherein the co-display protein is a transduction adjuvant that increases transduction efficiency of the lipid particle as compared to a lipid particle that does not have a co-display protein comprising a transduction adjuvant.

172. The producer cell of any of embodiments 143-171, wherein the co-display protein is a transduction adjuvant that binds to a surface molecule on target cells to enhance transduction.

173. The producer cell of embodiment 171 or 172 wherein the transduction adjuvant comprises
(i) an extracellular domain,
(ii) a linker, and
(iii) an anchor.

174. The producer cell of embodiment 173, wherein the extracellular domain of the transduction adjuvant binds an integrin binding domain.

175. The producer cell of embodiments 173 or 174, the extracellular domain of the transduction adjuvant comprises the extracellular domain of fibronectin, retronectin, or a functional variant thereof.

176. The producer cell of any of embodiments 165-175, wherein the linker is derived from a human protein.

177. The producer cell of any of embodiments 165-176, wherein the linker is a derived from human CD8α, IgG3 hinge, IgG1 hinge, IgG4 heavy chain, IgG4, or CD28.

178. The producer cell of any of embodiments 165-177, wherein the linker is modified to remove a cysteine residue.

179. The producer cell of any of embodiments 165-178, wherein the linker is rigid or wherein the linker is flexible.

180. The producer cell of any of embodiments 165-179, wherein the anchor is a peptide anchor that is a transmembrane domain.

181. The producer cell of any of embodiments 165-180, wherein the anchor is a transmembrane domain that is a transmembrane domain of a protein selected from PDGFR, VSV-G, CD8α, ICAM1, HLA-DRB, CD49d, or Transferrin.

182. The producer cell of any of embodiments 165-179, wherein the anchor is a GPI lipid anchor.

183. The producer cell of any of embodiments 143, 145-182, wherein the targeting moiety is selected from the group consisting of an antibody or antigen-binding fragment, an engineered binding domain, a nanobody, a DARPin, an Aptamer, an Affimer, an Affibody, a Knottin, an Avimer, a Monobody, an Anticalin, a Fynomer, and a targeting peptide.

184. The producer cell of any of embodiments 143, 145-183, wherein the targeting moiety binds to a cell surface molecule present on a target cell.

185. The producer cell of embodiment 184, wherein the cell surface molecule is a protein, glycan, or lipid.

186. The producer cell of any of embodiments 143, 145-185, wherein the target cell is selected from the group consisting of tumor-infiltrating lymphocytes, T cells, neoplastic or tumor cells, virus-infected cells, stem cells, central nervous system (CNS) cells, hematopoietic stem cells (HSCs), and liver cells.

187. The producer cell of any of embodiments 143, 145-186, wherein the target cells is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

188. The producer cell of any of embodiments 143, 145-187, wherein the target is a hematopoietic stem cell.

189. The producer cell of any of embodiment 188, wherein the cell surface molecule is selected from CD34, CD105, or CD117.

190. The producer cell of any of embodiments 143, 145-187, wherein the target cell is a T cell.

191. The producer cell of embodiment 190, wherein the cell surface molecule is selected from the group consisting of CD3, CD4, CD7 CD8, ASCT2, CD110, CD133, CD146, CD164, CD46, CD49f, CD90, EPCR, and ITGA3.

192. The producer cell of any of embodiments 143, 145-187, wherein the target cell is a hepatocyte.

193. The producer cell of embodiment 192, wherein the cell surface molecule is selected from the group consisting of ASGR1, ASGR2 and TM4SF5.

194. The producer cell of any of embodiments 143, 145-193, wherein the targeting moiety is selected from the group consisting of a single domain antibody or a single chain variable fragment (scFv).

195. The producer cell of embodiment 194, wherein the single domain antibody is a VHH.

196. The producer cell of any of embodiments 143, 145-195, wherein the retargeted attachment protein is a first attachment protein and the lipid particle further comprises a second retargeted attachment protein.

197. The producer cell of embodiment 196, wherein the second retargeted attachment protein comprises (i) a second paramyxovirus envelope attachment protein; and (ii) a second targeting moiety directed to a target molecule expressed on the surface of a target cell.

198. The producer cell of any of embodiments 196 or 197, wherein the second targeting moiety is selected from the group consisting of an antibody or antigen-binding fragment, an engineered binding domain, a nanobody, a DARPin, an Aptamer, an Affimer, an Affibody, a Knottin, an Avimer, a Monobody, an Anticalin, a Fynomer, and a targeting peptide.

199. The producer cell of embodiment 197 or 198, wherein the target cell is selected from the group consisting of tumor-infiltrating lymphocytes, T cells, neoplastic or tumor cells, virus-infected cells, stem cells, central nervous system (CNS) cells, hematopoietic stem cells (HSCs), and liver cells.

200. The producer cell of any of embodiments 197-199, wherein the target cells is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

201. The producer cell of any of embodiments 197-200, wherein the target is a hematopoietic stem cell.

202. The producer cell of any of embodiment 201, wherein the cell surface molecule is selected from CD34, CD105, or CD117.

203. The producer cell of any of embodiments 197-200, wherein the target cell is a T cell.

204. The producer cell of embodiment 203, wherein the cell surface molecule is selected from the group consisting of CD3, CD4, CD7 CD8, ASCT2, CD110, CD133, CD146, CD164, CD46, CD49f, CD90, EPCR, and ITGA3.

205. The producer cell of any of embodiments 197-200, wherein the target cell is a hepatocyte.

206. The producer cell of embodiment 205, wherein the cell surface molecule is selected from the group consisting of ASGR1, ASGR2 and TM4SF5.

207. The producer cell of any of embodiments 197-206, wherein the targeting moiety is selected from the group consisting of a single domain antibody or a single chain variable fragment (scFv).

208. The producer cell of embodiment 207, wherein the single domain antibody is a VHH.

209. The producer cell of embodiment 144, wherein the BaEV envelope glycoprotein is a truncated BaEV envelope glycoprotein comprising a cytoplasmic tail with a partial fusion inhibitory R peptide relative to the inhibitory R peptide of a wild-type BaEV envelope glycoprotein, wherein the partial fusion inhibitory R peptide comprises at least one amino-terminal amino acid but less than the full length of the inhibitory R peptide of the wild-type BaEV envelope glycoprotein.

210. The producer cell of embodiment 144 or 209, wherein the BaEV envelope glycoprotein is a truncated BaEV envelope glycoprotein comprising a cytoplasmic tail with a partial fusion inhibitory R peptide relative to a wild-type BaEV envelope glycoprotein, wherein the cytoplasmic tail is 25 amino acids in length and contains 8 contiguous amino-terminal acids of the inhibitory R peptide (R+8) of the full length inhibitory R peptide of wild-type BaEV envelope glycoprotein.

211. The producer cell of embodiment 209 or 210, wherein the truncated BaEV glycoprotein comprises: (i) a glycoprotein 70 (g70) subunit or a biologically active portion thereof, and (ii) a portion of the glycoprotein p20E (p20E) subunit comprising the cytoplasmic tail with the partial inhibitory R peptide.

212. The producer cell of embodiments 209-211, wherein the BaEV envelope glycoprotein binds an ASCT-2 or ASCT-1 receptor.

213. The producer cell of any of embodiments 209-212, wherein the truncated BaEV envelope glycoprotein is truncated relative to SEQ ID NO: 622 and lacks up to 16 contiguous amino acids from the C-terminal cytoplasmic tail of SEQ ID NO: 622.

214. The producer cell of any of embodiments 209-213, wherein the partial fusion inhibitory R peptide is set forth as amino acids 1 to 8 of SEQ ID NO: 623, optionally wherein the cytoplasmic tail is set forth in SEQ ID NO: 624 (R+8).

215. The producer cell of any of embodiments 209-214, wherein the truncated BaEV envelope glycoprotein is set forth in SEQ ID NO: 625.

216. The producer cell of any of embodiments 136, 138-208, wherein the paramyxovirus envelope attachment protein is an envelope attachment protein from a Nipah virus, Hendra virus, or Measles virus, or is a variant or biologically active portion thereof of any of the foregoing.

217. The producer cell of any of embodiments 143, 145-216, wherein the paramyxovirus envelope attachment protein is a wild-type paramyxovirus G protein, H protein or HN protein or is a variant or biologically active portion of any of the foregoing.

218. The producer cell of embodiment 216 or embodiment 217, wherein the paramyxovirus envelope attachment protein is a wild-type Nipah virus G (NiV-G) protein or is a variant or biologically active portion of a NiV-G.

219. The producer cell of embodiments 143, 145-218, wherein the paramyxovirus envelope attachment protein is a variant paramyxovirus envelope attachment protein.

220. The producer cell of embodiment 219, wherein the variant paramyxovirus envelope attachment protein comprises one or more mutations that reduces native tropism relative to the wild-type paramyxovirus envelope attachment protein not comprising the one or more mutations.

221. The producer cell of any of embodiments 143, 145-220, wherein the paramyxovirus envelope attachment protein is a variant NiV-G that is a variant or a biologically active portion of a wild-type NiV-G.

222. The producer cell of any one of embodiments 218-221, wherein the variant is a variant NiV-G that is a variant of a wild-type Nipah virus G (NiV-G) protein or a biologically active portion thereof.

223. The producer cell of embodiment 221 or 222, wherein the variant NiV-G is truncated by up to 40 contiguous amino acids at or near the N-terminus of the wild-type NiV-G set forth in SEQ ID NO:1.

224. The producer cell of any of embodiments 218-223, wherein the variant NiV-G has a truncation of amino acids 2-34 of the wild-type NiV-G set forth in SEQ ID NO:1.

225. The producer cell of any of embodiments 218-224, wherein the variant NiV-G exhibits reduced binding to Ephrin B2 or Ephrin B3.

226. The producer cell of embodiment 225, wherein the variant NiV-G comprises: one or more amino acid substitutions corresponding to amino acid substitutions selected from the group consisting of E501A, W504A, Q530A and E533A with reference to numbering set forth in SEQ ID NO:1.

227. The producer cell of embodiment 225 or embodiment 226, wherein the variant NiV-G comprises amino acid substitutions E501A, W504A, Q530A and E533A with reference to numbering set forth in SEQ ID NO:1.

228. The producer cell of any of embodiments 218-227, wherein the variant NiV-G has the amino acid sequence set forth in SEQ ID NO: 228 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:228.

229. The producer cell of any of embodiments 218-228, wherein the variant NiV-G has the amino acid sequence set forth in SEQ ID NO:228.

230. The producer cell of any of embodiments 143-229, wherein the particle further comprises an additional attachment protein that is a paramyxovirus envelope attachment protein that is a variant NiV-G that is a variant or a biologically active portion of a wild-type NiV-G.

231. The producer cell of embodiment 230, wherein the variant NiV-G exhibits reduced binding to Ephrin B2 or Ephrin B3.

232. The producer cell of embodiment 231, wherein the variant NiV-G comprises: one or more amino acid substitutions corresponding to amino acid substitutions selected from the group consisting of E501A, W504A, Q530A and E533A with reference to numbering set forth in SEQ ID NO:1.

233. The producer cell of any of embodiments 143-232, wherein the at least one paramyxovirus fusion (F) protein is an F protein from a henipavirus or is a biologically active portion thereof or variant thereof.

234. The producer cell of embodiment 233, wherein the henipavirus is a Hendra virus.

235. The producer cell of embodiment 233, wherein the henipavirus is a Nipah virus.

236. The producer cell of any of embodiments 143, 145-208, and 216-235, wherein the paramyxovirus F protein is a wild-type NiV-F protein or a variant or a biologically active portion thereof.

237. The producer cell of any of embodiments 143-236, wherein the paramyxovirus F protein is a variant NiV-F that is a variant or a biologically active portion of a wild-type NiV-F protein.

238. The producer cell of embodiment 237, wherein the variant NiV-F is truncated by up to 22 contiguous amino acids the at the C-terminus of the wild-type NiV-F set forth in SEQ ID NO:235, optionally not include the initial methionine.

239. The producer cell of embodiment 237 or embodiment 238, wherein the variant NiV-F protein is a truncated NiV-F that lacks amino acids 525-546 of SEQ ID NO:235.

240. The producer cell of any of embodiments 237-239, wherein the variant NiV-F has the amino acid sequence set forth in SEQ ID NO: 227 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:227.

241. The producer cell of any of embodiments 237-240, wherein the variant NiV-F has the amino acid sequence set forth in SEQ ID NO:227.

242. The producer cell of any of embodiments 143-241, wherein the paramyxovirus F protein is an F0 precursor or is a proteolytically cleaved form thereof comprising F1 and F2 subunits.

243. The producer cell of embodiment 242, wherein the proteolytically cleaved form is a cathepsin L cleavage product.

244. The producer cell of any of embodiments 143-243, wherein the targeting moiety and the paramyxovirus envelope attachment protein or biologically active portion thereof is attached via a linker.

245. The producer cell of embodiment 244, wherein the linker is a peptide linker.

246. The producer cell of embodiment 245, wherein the peptide linker is 2 to 65 amino acids in length.

247. The producer cell of embodiment 245 or embodiment 246, wherein the peptide linker is a flexible linker that comprises GS, GGS, GGGGS, GGGGGS or combinations thereof.

248. The producer cell of any of embodiments 245-247, wherein the peptide linker is selected from: (GGS)n, wherein n is 1 to 10; (GGGGS)n, wherein n is 1 to 10; or (GGGGGS)n, wherein n is 1 to 6.

249. The producer cell of any of embodiments 143-248, wherein the paramyxovirus fusion (F) protein exhibits fusogenic activity with a target cell upon binding of at least one paramyxovirus envelope attachment protein o to the target molecule on the target cell.

250. The producer cell of any of embodiments 143-249, wherein the lipid particle comprises a viral nucleic acid.

251. The producer cell of embodiment 250, wherein the viral nucleic acid comprises one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

252. The producer cell of any of embodiments 143-251, wherein the lipid particle is a viral vector.

253. The producer cell of any of embodiments 143-252, that is a retroviral vector.

254. The producer cell of any of embodiments 143-253, that is a lentiviral vector.

255. The producer cell of any of embodiments 143-249, wherein the lipid particle is devoid of viral genomic DNA.

256. The producer cell of any of embodiments 143-249 and 225, that is a viral-like particle.

257. The producer cell of any of embodiments 143-249, 225, and 256, that is a retroviral-like particle.

258. The producer cell of any of embodiments 143-249, 225, and 256, that is a lentiviral-like particle.

259. The producer cell of any of embodiments 143-258, wherein the lipid particle is produced as a preparation with increased titer compared to a reference lipid particle preparation that is similarly produced but with only the first retargeted attachment protein.

260. The producer cell of embodiment 259, wherein the titer is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.

261. The producer cell of any of embodiments 143-260, further comprising an exogenous agent for delivery to a target cell.

262. The producer cell of embodiment 261, wherein the exogenous agent is present in the lumen.

263. The producer cell of embodiment 261 or embodiment 262, wherein the exogenous agent is a protein or a nucleic acid, optionally wherein the nucleic acid is a DNA or RNA.

264. The producer cell of any of embodiments 261-263, wherein the exogenous agent is a nucleic acid encoding a cargo for delivery to the target cell.

265. The producer cell of any of embodiments 261-264, wherein the exogenous agent is or encodes a therapeutic agent, a diagnostic agent or a genome-modifying enzyme.

266. The producer cell of any of embodiments 261-265, wherein the exogenous agent encodes a membrane protein, optionally wherein the membrane protein is an antigen receptor for targeting cells expressed by or associated with a disease or condition.

267. The producer cell of embodiment 266, wherein the membrane protein is a chimeric antigen receptor (CAR).

268. The producer cell of any of embodiments 261-267, wherein the exogenous agent is a nucleic acid comprising a payload gene for correcting a genetic deficiency, optionally a genetic deficiency in the target cell, optionally wherein the genetic deficiency is associated with a liver cell or a hepatocyte.

269. The producer cell of any of embodiments 261-268, wherein binding of the paramyxovirus envelope attachment protein or biologically active portion thereof to a target molecule expressed on the surface of a target cell mediates fusion of the particle with the target cell and delivery of the exogenous agent to the target cell.

270. The producer cell of any of embodiments 261-269, wherein at or greater than 10%, 20%, 30%, 40%, 50%, 60% of the target cells are delivered the exogenous agent.

271. The producer cell of any of embodiments 261-270, wherein delivery of the exogenous cell to the target cell is increased compared to a reference particle preparation that is similarly produced but with only a retargeted attachment protein (e.g., no co-display protein).

272. The producer cell of embodiment 271, wherein the delivery to the target cell is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.

273. A method of making a lipid particle, comprising:
a) providing a producer cell of any of embodiments 143-272;
b) culturing the cell under conditions that allow for production of the lipid particle, and
c) separating, enriching, or purifying the lipid particle from the cell, thereby making the lipid particle.

274. The method of embodiment 273, wherein the lipid particle is a pseudotyped lentiviral vector.

275. A lipid particle produced by the method of embodiment 273 or embodiment 274.

276. A composition comprising a plurality of lipid particles of any of embodiments 1-142 and 275.

277. The composition of embodiment 276 further comprising a pharmaceutically acceptable carrier.

278. A method of transducing a cell comprising contacting a cell with a lipid particle of any of embodiments 1-142 and 276 or a composition of embodiment 276 or embodiment 277.

279. A method of delivering an exogenous agent into a target cell, the method comprising contacting a lipid particle of any of embodiments 131-142 and 275 or a composition of embodiment 276 or embodiment 277 with a target cell.

280. The method of embodiment 278 or embodiment 279, wherein the contacting is in vitro or ex vivo.

281. The method of embodiment 278 or embodiment 279, wherein the contacting is in vivo in a subject.

282. A method of delivering an exogenous agent to a cell in a subject, the method comprising administering to the subject a lipid particle of any of embodiments 131-142 and 275 or a composition of embodiment 276 or embodiment 277.

283. The method of embodiment 282, wherein the exogenous agent is or encodes a therapeutic agent for treating a disease or condition in the subject.

284. A method of treatment, the method comprising administering to a subject a lipid particle of any of embodiments 131-142 and 275 or a composition of embodiment 276 or embodiment 277.

285. The method of any of embodiments 279-284, wherein the exogenous agent is or encodes a membrane protein, optionally a chimeric antigen receptor, for targeting an antigen associated with a disease or condition in the subject.

286. The method of any of embodiments 279-285, wherein the exogenous agent is for use in gene therapy to correct a genetic deficiency or replaces a deficient or missing gene in the subject.

287. The method of any of embodiments 279-286, wherein the subject is a human subject.

288. A co-display polypeptide, comprising one or more of:
a leader domain;
one or more fibronectin fragments;
a hinge domain;
an anchor domain; and
a intracellular domain.

289. The co-display polypeptide of embodiment 288, wherein the co-display polypeptide is partially derived or derived from one or more recombinant fibronectin fragments.

290. The co-display polypeptide of embodiment 288 or 289, wherein the one or more recombinant fibronectin fragments are selected from: CH296-A, CH296-B, CH296-C, CH296-D, CH296-E, and CH296-F.

291. A co-display polypeptide, comprising one or more of:
a leader domain;
a CS-1 domain;
a linker;
a C-domain;
a hinge domain;
an anchor domain; and
a intracellular domain.

### VIII. EXAMPLES

The following examples are included for illustrative purposes only and are not intended to limit the scope of the invention.

### Example 1 Development and In Vitro Testing of CD3 Co-Display Construct

Lentiviral vector (LV) was pseudotyped with a re-targeted fusogen and CD3 co-display protein to generate a co-display lentiviral vector and used to assess T cell activation and frequency of transduced cells.

In this example, LV was pseudotyped with a re-targeted fusogen targeting CD8. The CD8-retargeted fusogen contained an anti-CD8 scFv targeting human CD8α (set forth in SEQ ID NO: 801) as a fusion to the C-terminus of the exemplary Nipah G (NiV-G) protein sequence GcΔ34 that was mutated to ablate its native tropism (Bender et al. 2016 PLoS Pathol 12(6):e1005641; set forth in SEQ ID NO:228). The lentiviral vector also was pseudotyped with the exemplary Nipah F (NiV-F) protein sequence NivFdel22 (SEQ ID NO:226; or SEQ ID NO:227 without a signal sequence; Bender et al. 2016 PLoS).

The LV was also generated to be pseudotyped with an anti-CD3 co-display protein. An exemplary pseudotyped vector is shown with an exemplary co-display protein in FIG. 1. A total of 75 different CD3 co-display constructs were generated as shown in Table E3, in which two exemplary scFv CD3 binders 1 and 2 as set forth in SEQ ID NOs: 482 and 802, respectively were tested with combinations of 12 different linkers and 8 different anchor domains as set forth in the tables below. The linkers were chosen as representative of variable flexibility, length, and potential for oligomerization, while the anchor domains included viral fusogen anchor domains as well as lipid anchors.

TABLE E1
Exemplary Linkers for CD3 Co-display Protein

| | | |
|---|---|---|
| GGGGS, (GGGGS)3 | SEQ ID NO 232 | Flexible, adjustable length |
| EAAAAK, (EAAAAK)3 | SEQ ID NO 780 | Rigid, helical, adjustable |
| | | length |
| Mod Human CD8a | SEQ ID NO 781 | Used in published UMTx |
| | | OKT3 co-display |
| Human IgG3 U Hing | SEQ ID NO 783 | Human derived |
| (with cys) | | |
| Human IgG3 U Hing | SEQ ID NO: | Human derived |
| (w/o cys) | 783 w/o cys | |
| Human IgG1 UML | SEQ ID NO 784 | Human derived |
| Hinge (with cys) | | |
| Human IgG1 UML | SEQ ID NO 784 | Human derived |
| Hinge (w/o cys) | w/o cys | |
| IgG4 heavy chain seq | SEQ ID NO 803 | Human derived |
| (with cys) | | |
| IgG4 heavy chain seq | SEQ ID NO 803 | Human derived |
| (w/o cys) | w/o cys | |
| CD28 extension | SEQ ID NO 791 | Human derived |
| IgG4 (with cys) | SEQ ID NO 790 | Human derived |
| IgG4 (w/o cys) | SEQ ID NO 790 | Human derived |
| | w/o cys | |

TABLE E2
Exemplary Anchors for CD3 Co-display Protein

| | |
|---|---|
| PDGFR | SEQ ID NO 785 |
| GPI anchor | SEQ ID NO 793 |
| VSV-G Anchor | SEQ ID NO 786 |
| CD8a TM | SEQ ID NO 787 |
| ICAM1 | SEQ ID NO 782 |
| HLA-DRB | SEQ ID NO 788 |
| CD49d | SEQ ID NO 789 |
| Transferrin | SEQ ID NO 794 |
| IgG4 + PDGRFstalk | SEQ ID NO 792 |

TABLE E3
Exemplary CD3+Fusogen Co-display Constructs

| | SEQ ID | SEQ ID |
|---|---|---|
| | Nucleic | Amino |
| Construct name | Acid | Acid |
| HA-CD3.s1-IgG1ser-PDGFR | 626 | 703 |
| HA-CD3.s1-IgG1ser-GPI | 627 | 704 |
| HA-CD3.s1-IgG1ser-VSVGan | 628 | 705 |
| HA-CD3.s1-IgG1ser-CD8aTm | 629 | 706 |
| HA-CD3.s1-IgG1ser-ICAM1 | 630 | 707 |
| HA-CD3.s1-IgG1ser-HLADRB | 631 | 708 |
| HA-CD3.s1-IgG1ser-CD49d | 632 | 709 |
| TFR1-IgG1ser-CD3.s1-HA | 633 | 710 |
| HA-CD3.s1-EAAAKx3-PDGFR | 634 | 711 |
| HA-CD3.s1-EAAAKx3-GPI | 635 | 712 |
| HA-CD3.s1-EAAAKx3-VSVGan | 636 | 713 |
| HA-CD3.s1-EAAAKx3-CD8aTm | 637 | 714 |
| HA-CD3.s1-EAAAKx3-HLADRB | 638 | 715 |
| HA-CD3.s1-EAAAKx3-CD49d | 639 | 716 |
| TFR1-EAAAKx3-CD3.s1-HA | 640 | 717 |
| HA-CD3.s2-IgG1ser-PDGFR | 641 | 718 |
| HA-CD3.s2-IgG1ser-GPI | 642 | 719 |
| HA-CD3.s2-IgG1ser-VSVGan | 643 | 720 |
| HA-CD3.s2-IgG1ser-ICAM1 | 644 | 721 |
| HA-CD3.s2-IgG1ser-HLADRB | 645 | 722 |
| HA-CD3.s2-IgG1ser-CD49d | 646 | 723 |
| TFR1-IgG1ser-CD3.s2-HA | 647 | 724 |
| HA-CD3.s2-EAAAKx3-PDGFR | 648 | 725 |
| HA-CD3.s2-EAAAKx3-GPI | 649 | 726 |
| HA-CD3.s2-EAAAKx3-VSVGan | 650 | 727 |
| HA-CD3.s2-EAAAKx3-CD8aTm | 651 | 728 |
| HA-CD3.s2-EAAAKx3-HLADRB | 652 | 729 |
| HA-CD3.s2-EAAAKx3-CD49d | 653 | 730 |
| TFR1-EAAAKx3-CD3.s2-HA | 654 | 731 |
| HA-CD3.s1-G4SP-DGFR | 655 | 732 |
| HA-CD3.s1-AEAAAK-PDGFR | 656 | 733 |
| HA-CD3.s1-IgG3ser-PDGFR | 657 | 734 |
| HA-CD3.s1-CD28-PDGFR | 658 | 735 |
| HA-CD3.s1-IgG3hng-PDGFR | 659 | 736 |
| HA-CD3.s1-IgG1hng-PDGFR | 660 | 737 |
| HA-CD3.s1-IgG4g-PDGFR | 661 | 738 |
| HA-CD3.s1-G4SICAM1 | 662 | 739 |
| HA-CD3.s1-G4Sx3-ICAM1 | 663 | 740 |
| HA-CD3.s1-AEAAAK-ICAM1 | 664 | 741 |
| HA-CD3.s1-CD8ah-ICAM1 | 665 | 742 |
| HA-CD3.s1-IgG3ser-ICAM1 | 666 | 743 |
| HA-CD3.s1-IgG4-ser-ICAM1 | 667 | 744 |
| HA-CD3.s1-CD28-ICAM1 | 668 | 745 |
| HA-CD3.s1-IgG3hng-ICAM1 | 669 | 746 |
| HA-CD3.s1-IgG1hng-ICAM1 | 670 | 747 |
| HA-CD3.s1-IgG4g-ICAM1 | 671 | 748 |
| HA-CD3.s2-G4SPDGFR | 672 | 749 |
| HA-CD3.s2-G4Sx3-PDGFR | 673 | 750 |
| HA-CD3.s2-AEAAAK-PDGFR | 674 | 751 |
| HA-CD3.s2-CD8ah-PDGFR | 675 | 752 |
| HA-CD3.s2-IgG3ser-PDGFR | 676 | 753 |
| HA-CD3.s2-IgG4-ser-PDGFR | 677 | 754 |
| HA-CD3.s2-CD28-PDGFR | 678 | 755 |
| HA-CD3.s2-IgG3hng-PDGFR | 679 | 756 |
| HA-CD3.s2-IgG1hng-PDGFR | 680 | 757 |
| HA-CD3.s2-IgG4g-PDGFR | 681 | 758 |
| HA-CD3.s2-G4SICAM1 | 682 | 759 |
| HA-CD3.s2-G4Sx3-ICAM1 | 683 | 760 |
| HA-CD3.s2-AEAAAK-ICAM1 | 684 | 761 |
| HA-CD3.s2-CD8ah-ICAM1 | 685 | 762 |
| HA-CD3.s2-IgG3ser-ICAM1 | 686 | 763 |
| HA-CD3.s2-CD28-ICAM1 | 687 | 764 |
| HA-CD3.s2-IgG3hng-ICAM1 | 688 | 765 |
| HA-CD3.s2-IgG1hng-ICAM1 | 689 | 766 |
| HA-CD3.s2-IgG4g-ICAM1 | 690 | 767 |
| RevisedHA-UMTxOKT3 | 691 | 768 |
| RevisedOKT3 | 692 | 769 |
| HA-ASGR1.scFv-IgG1ser-PDGF | 693 | 770 |
| pCAGGS-SS-GGlandingpad-RFP | 694 | 771 |
| HA-CD3.s1-IgG4PDGFR-Dob | 695 | 772 |
| HA-CD3.s1-IgG4PDGFR-Dob2 | 696 | 773 |
| HA-CD3.s1-EAAAKx3-ICAM1 | 697 | 774 |
| HA-CD3.s2-IgG1ser-CD8aTm | 698 | 775 |
| HA-CD3.s2-EAAAKx3-ICAM1 | 699 | 776 |
| HA-CD3.s1-G4Sx3-PDGFR | 700 | 777 |
| HA-CD3.s1-CD8ah-PDGFR | 701 | 778 |
| HA-CD3.s2-IgG4-ser-ICAM1 | 702 | 779 |

For producing the lentiviral vector, plasmids encoding the re-targeted NiV-G fusion protein, the NiV-F protein, and the co-display protein were transfected into the same population of HEK 293 cells. The cells also were transfected with packaging plasmid for viral vector production (e.g. gag/pol, rev), and a transfer plasmid encoding enhanced green fluorescent protein (eGFP). Following viral vector production, the cell culture was centrifuged to pellet the cells and the supernatant containing crude virus was collected.

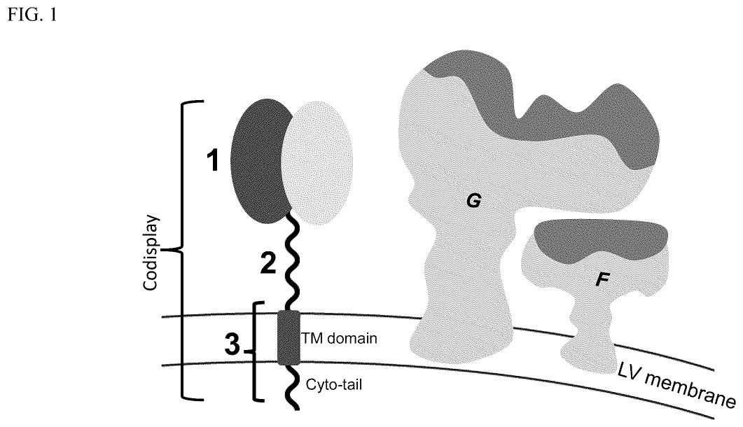

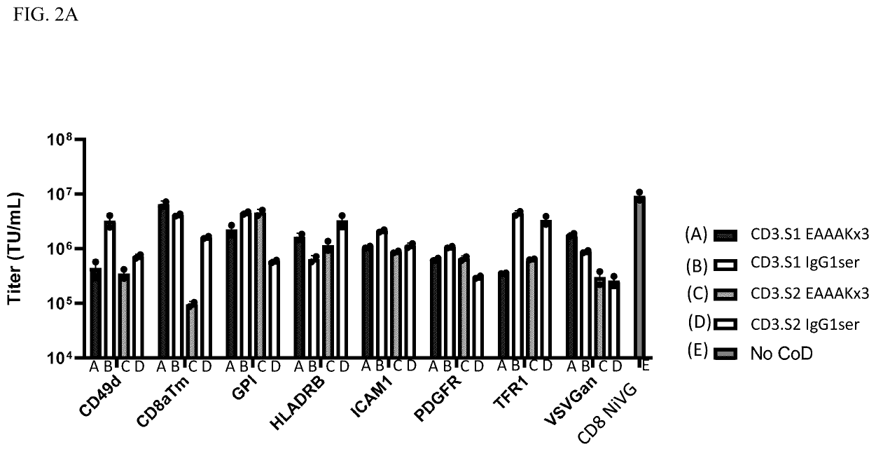

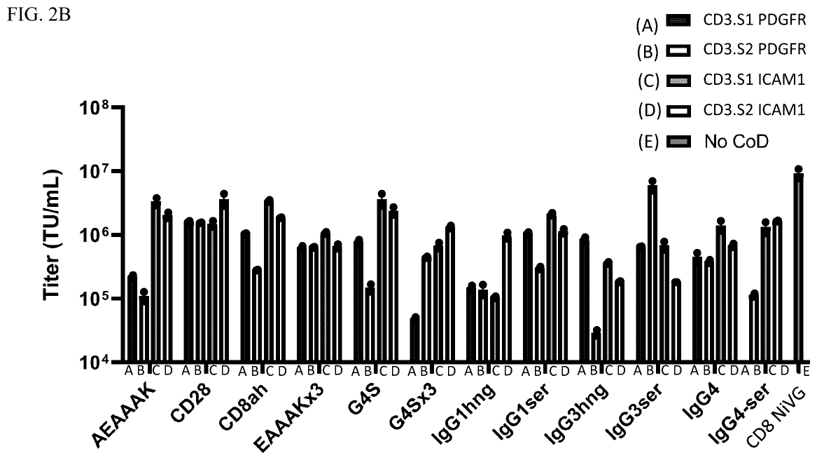

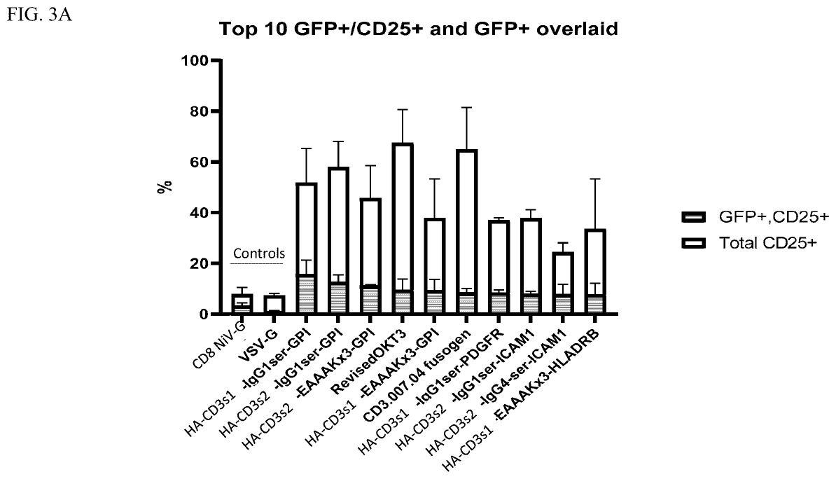

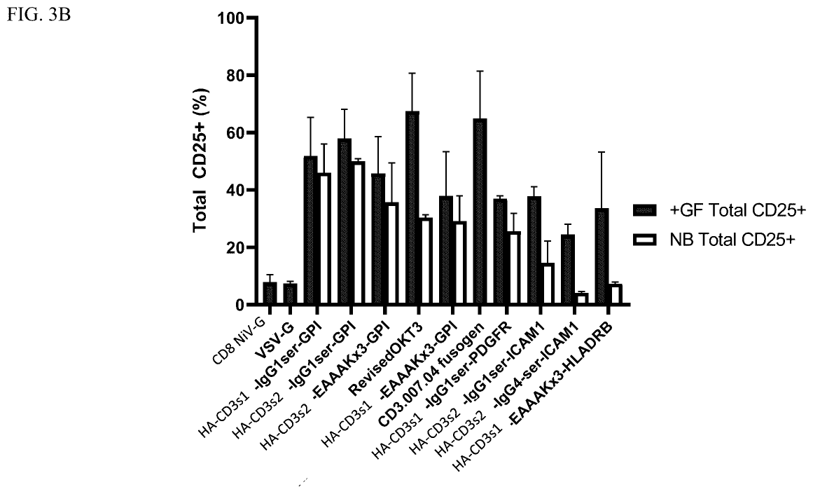

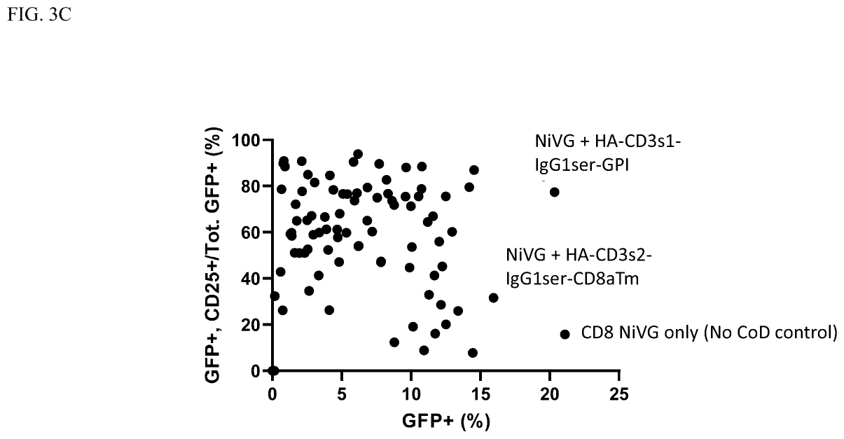

Titer was assessed using SupT1 T cells as shown in FIG. 2. Without wishing to be bound by theory, it is considered that SupT1 titers are directly related to the activity CD8 retargeted fusogen in the presence of the co-display protein. It was observed that some of the CD3 codisplay constructs negatively impacted SupT1 titers, and some had minimal impact on SupT1 titers, either when sorted by anchor domain in FIG. 2A or by linker domain in FIG. 2B. Lead CD3 codisplay constructs were selected based on minimal impact to SupT1 titers.

Transduction efficiency of the produced pseudotyped lentivirus preparations for recipient T cells was assessed by monitoring GFP expression by flow cytometry. Transduction efficiency of the CD8-targeted LV preparation featuring co-display of CD3 binders was compared to reference pseudotyped lentiviral vectors, including a vector expressing only a CD8 scFv, VSV-G, Cocal-G, Cocal-G+CD3 (OKT3)-scFv, CD3 scFv. Activation was similarly assessed by monitoring for CD25 and/or CD69 expression.

Crude pseudotyped LV preparations were transduced into recipient PanT cells (StemCell Technologies, donor 190981203C) by single point dilutions followed by analysis of transduced cells for GFP expression and/or activation marker expression (e.g., CD25) by flow cytometry.

Transduction of PanT cells is shown in FIG. 3A. The results showed that the LV pseudotyped with a CD8 retargeted fusogen and co-displaying a CD3 scFv efficiently activated T cells, including non-target T cells. This observed transduction was specific to only the target CD8+ T cells. The CD8 retargeted fusogen with CD3 codisplay did NOT transduce non-target T cells. Similar experiments shown in FIG. 3B demonstrate the comparative activation of CD3 co-display in vector which also expresses a CD8 retargeted fusogen as above. Two exemplary co-display candidates, constructs A and B set forth in Table E4, were observed to generally preserve overall PanT transduction and activate a high percentage of transduced cells (FIG. 3C).

TABLE E4
Exemplary CD3 Co-display Protein Constructs

| CD3 Cod | | | | | NA SEQ | AA SEQ |
|---|---|---|---|---|---|---|
| Construct | Fusogen | Co-display | Linker | Anchor | ID NO | ID NO |
| CD3 | anti-CD8 scFv | HA-CD3 scFv | IgG1ser | GPI | 627 | 704 |
| Construct A | targeting | (e.g., SEQ | | | | |
| | human CD8α (e.g., | ID NO: 482) | | | | |
| | SEQ ID NO 801) | | | | | |
| CD3 | anti-CD8 scFv | HA-CD3 scFv | IgG1ser | CD8a | 629 | 706 |
| Construct B | targeting | (e.g., SEQ | | TM | | |
| | human CD8α (e.g., | ID NO: 802) | | | | |
| | SEQ ID NO 801) | | | | | |

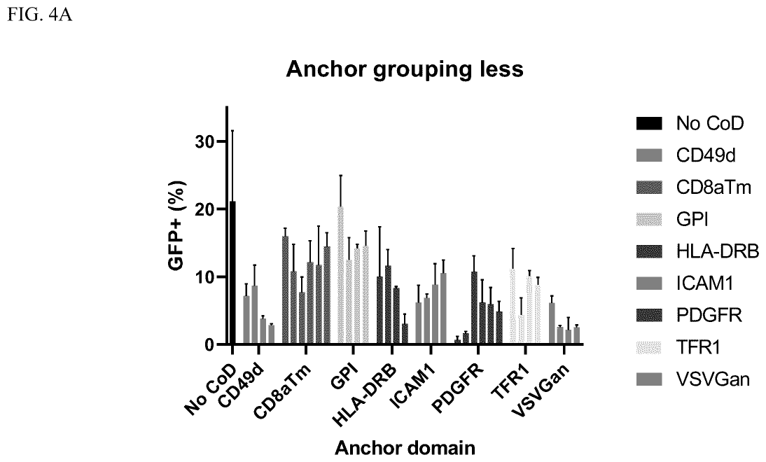

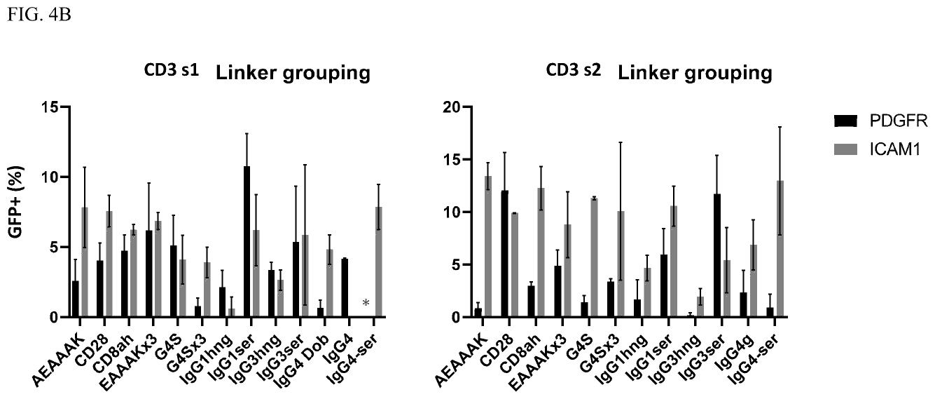

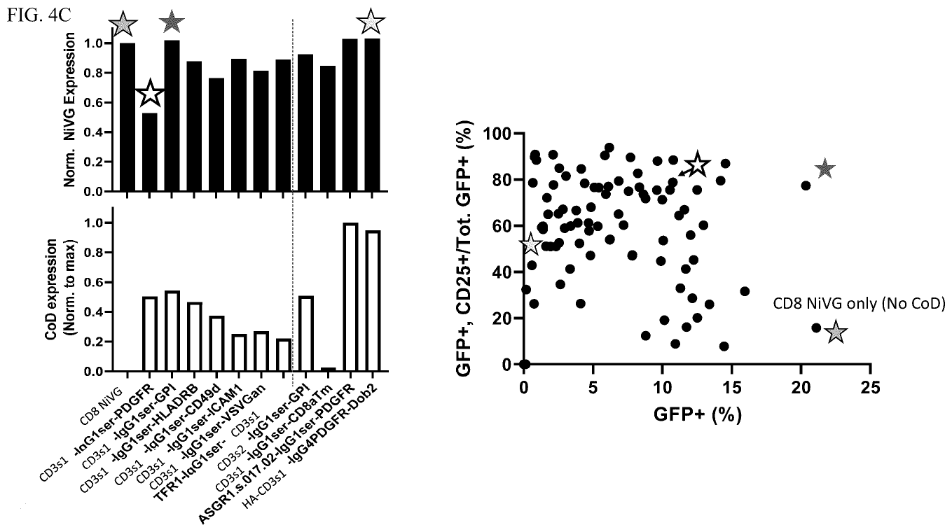

The impact of the co-display anchor domain as well as linked domain on transduction of Pan T cells was also assessed. Transduction efficiency of the produced pseudotyped lentivirus preparations for recipient T cells was assessed by monitoring GFP expression by flow cytometry. GFP expression as a percentage of total cells are shown in FIG. 4A as grouped by anchor domain, and FIG. 4B shows GFP expression per grouping by linker domain for each of the two CD3 targeted scFvs (as set forth in SEQ ID NOs: 482 and 802).

Next, the level of CD8-targeted Nipah G protein and CD3 co-display protein were assessed on the surface of the LV particles. As shown in FIG. 4C., some CD3 co-display constructs impact NiV-G expression levels and similarly had an observed effect on transduction efficiency (right panel). Without wishing to be bound by theory, the exemplary lead construct maintains G expression while expressing the CD3 co-display protein at a sufficient level for efficient activation.

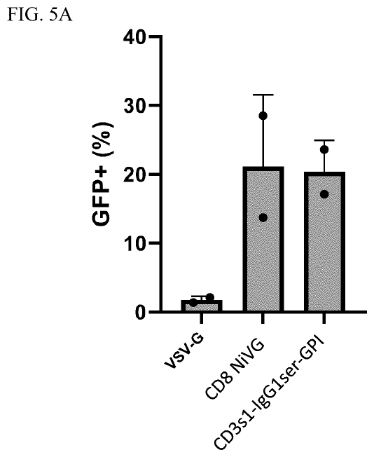

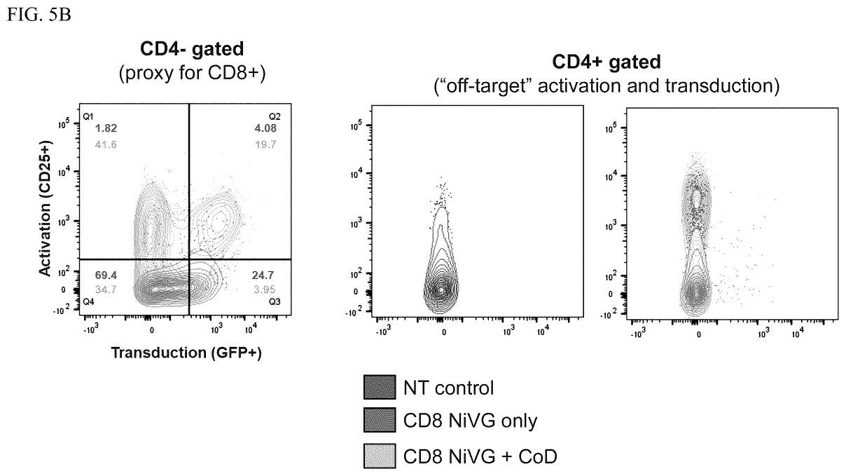

From these data, exemplary LV containing construct (HA-CD3.s.1-IgG1ser-GPI; CD3 Construct A) was further specifically assessed for potency and transduction efficiency on resting T cells. As shown in FIG. 5A, LV containing Construct A resulted in more GFP positive T cells (e.g., successful transduction of the GFP gene) than T cells transduced with LV pseudotyped with VSV-G. Further in FIG. 5B, CD3 co-display Construct A showed no increase in off-target (CD8−) transduction. However, activation of off-target cells was seen.

Further experiments were conducted with CD3 co-display Construct A—including in the delivery of a CD19-CAR transgene for T cell transduction and B cell killing as described below. For this, Pan T cells were first thawed in IL-2 containing media before being transduced via spinfection with or without nevirapine and with dilutions of viral vector comprising the co-display constructs listed in Table E5 below. Cell samples were collected 4 days post transduction, 7 days post transduction, and 10 days post transduction. As shown in Table E6, consistent titers were observed across the generated constructs and controls.

TABLE E5
Co-display constructs for CAR transgene delivery

| Name | Binder | Co-display (CoD) | Transgene |
|---|---|---|---|
| NivG-CD3 (GFP) | CD3 | N/A | GFP |
| NivG-CD8scFv (GFP) | CD3-CD8 | N/A | GFP |
| NivG-CD8scFv + CoD-CD3 (GFP) | CD3-CD8 | CD3 | GFP |
| (e.g., Construct A + GFP) | | | |
| NivG-CD3 (EF1a-FwdCAR) | CD3 | N/A | EF1a-FwdCAR |
| NivG-CD8 scFv (EF1a-FwdCAR) | CD3-CD8 | N/A | EF1a-FwdCAR |
| NivG-CD8 scFv + CoD-CD3 (EF1a- | CD3-CD8 | CD3 | EF1a-FwdCAR |
| FwdCAR) (e.g., Construct A + CAR) | | | |

TABLE E6
Co-display constructs and T cell titer

| Name | Titer (IU/mL) | PanT Titer (IU/mL) |
|---|---|---|
| NivG-CD3 (GFP) | | 2.52e+7 |
| NivG-CD8scFv (GFP) | | 2.58e+7 |
| NivG-CD8scFv + CoD-CD3 (GFP) | 1.46e8 | 2.13e+7 |
| (e.g., Construct A + GFP) | | |
| NivG-CD3 (EF1a-FwdCAR) | 3.22e7 | 4.38e+7 |
| NivG-CD8scFv (EF1a-FwdCAR) | 1.24e8 | 3.95e+7 |
| NivG-CD8scFv + CoD-CD3 (EF1a- | 8.46e7 | 5.67e+7 |
| FwdCAR) (e.g., Construct A + CAR) | | |
| NivG-CD8scFv + EF1a-FwdCAR- | 1.1e8 | 8.46e+6 |
| control | | |
| NivG-CD8scFv + EF1a-FwdCAR- | 1.1e8 | 2.01e+7 |
| control CD3/28 | | |

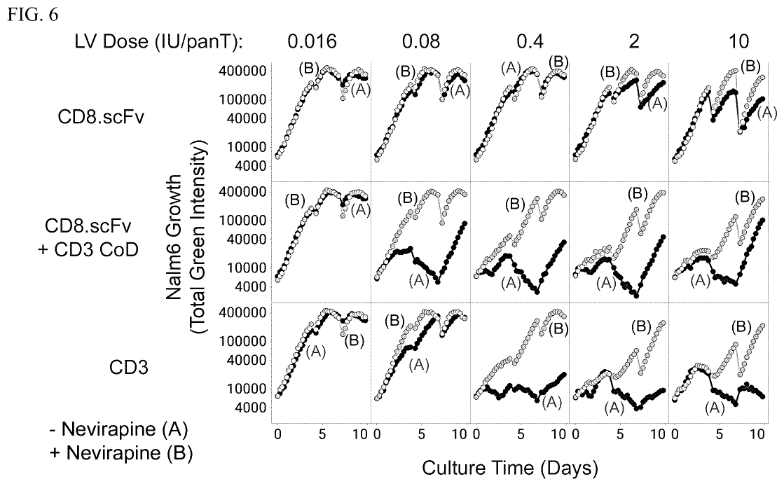

Nalm6 tumor cell growth was also assessed in the presence of co-display Construct A as well as CD8 scFv and CD3 scFv protein controls with a CAR transgene. Here, viral vector with CD3 codisplay as described above were generated to further comprise a CD 19 targeted CAR transgene before used to transduce T cells. Transduced cells were then incubated with Nalm6 tumor cells and resultant tumor cell killing was assessed. As shown in FIG. 6, the addition of the CD3 co-display protein such as in Construct A showed improved Nalm6 killing compared to the no CD3 co-display control, especially at 0.08 IU/panT dose. At higher doses, the CD3 co-display showed Nalm6 tumor control but also transiently increased allogenic reactivity and improved Nalm6 killing in a time dependent manner.

### Example 2 Development and In Vitro Testing of Retronectin Co-Display Construct

Lentiviral vector (LV) was pseudotyped with a fusogen and retronectin co-display protein to generate a co-display lentiviral vector and used to assess improved lentiviral transduction and gene editing of resting HSCs.

In this example, LV was pseudotyped with a BaEVTR fusogen, which has high tropism to CD34+ cells, along with expression of a retronectin co-display protein and packaged with gag-Cas9 cargo and B2M gRNA. The BaEVTR fusogen was generated as a variant fusogen, wherein the protease site between the T (cytoplasmic) and R (inhibitory) domains was mutated (SEQ ID NO: 797).

A library screen of retronectin peptides as set forth below in Table E7 was conducted using three display vectors during lipid particle production: HAMLV (Hemagglutinin glycoprotein with MLV signal peptide), pD (IgK leader sequence w/PDGRF transmembrane domain), and pD+linker (pDisplay with extracellular linker). Two plasmid ratios were tested for incorporating the retronectin co-display sequence at transfection of the lentiviral vector. A control was similarly assessed, “tx control”, which included empty DNA sequence. Crude vector was produced substantially as described above and used to transduce mobilized peripheral blood human CD34+ primary cells.

TABLE E7
Exemplary Retronectin Peptides

| | |
|---|---|
| CH296-A | Just C-Domain |
| CH296-B | 3G4S linker → C-Domain |
| CH296-C | Just CS-1 |
| CH296_D | 3G4S linker → CS-1 |
| CH296_E | C-Domain → 3G4S → CS-1 |
| CH296_F | CS-1 → 3G4S → C-Domain |

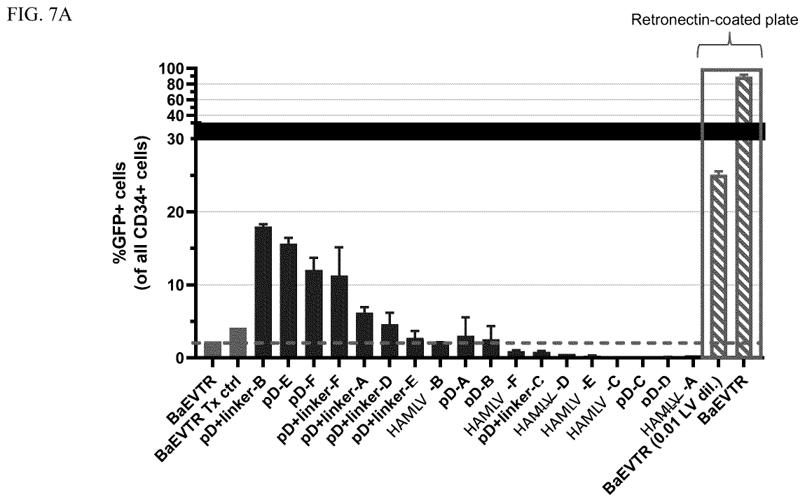

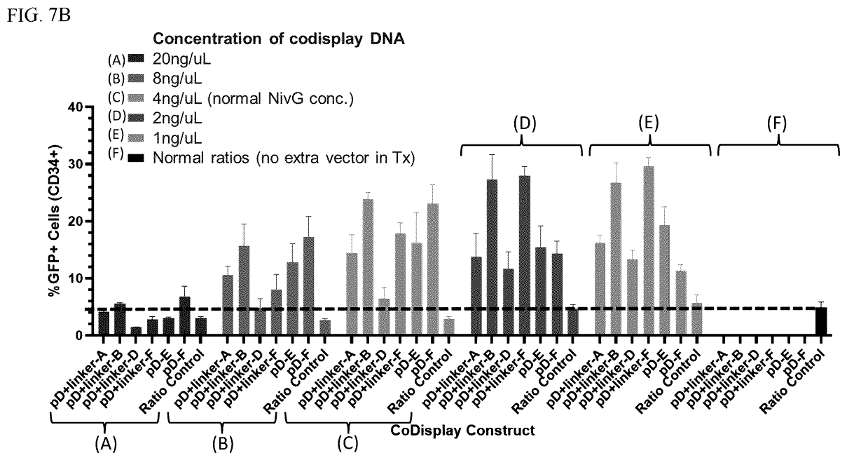

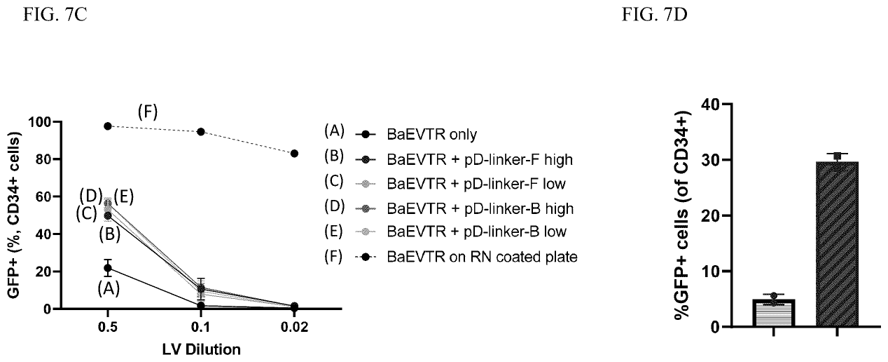

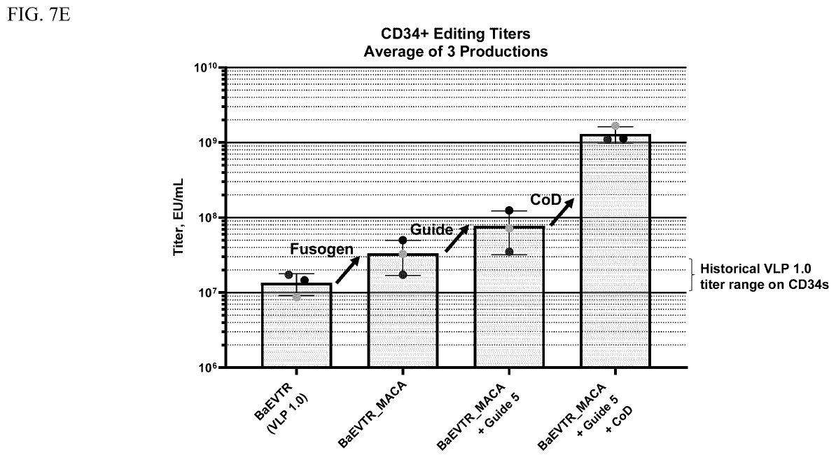

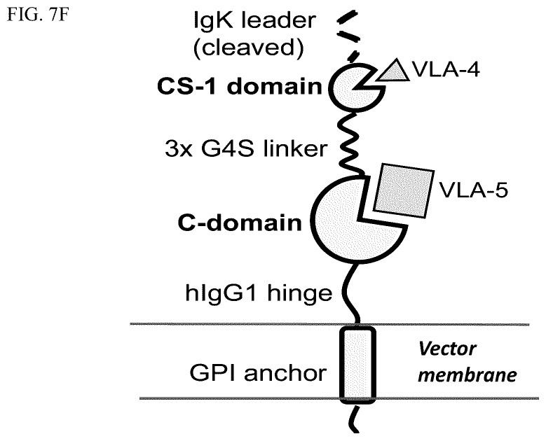

As shown in FIG. 7A, retronectin co-display on BaEVTR LV particles increased transduction over control constructs not comprising the retronectin co-display protein. For instance, retronectin co-display construct CH296-B (3G4S linker with C-domain) in a pD+linker expression vector resulted in pseudotyped LV with an increase in transduction 4-8× higher than the control BaEVTR groups expressing no co-display protein. It was generally observed that retronectin co-display constructs that increased transduction included the C-domain of the retronectin peptide and were with the pDisplay expression vector comprised within the produced lipid particle (i.e., viral vector).

Plasmid ratios were varied to assess impact on transduction efficiency of the retronectin co-display constructs. As shown in FIG. 7B., a correlation between lower co-display expression plasmid and higher transduction was observed.

Exemplary retronectin co-display fragment F (CH296_F) was then cloned into a separate co-display construct with GPI as the anchor/transmembrane domain (SEQ ID NO: 799). As demonstrated in FIG. 7C., retronectin co-display protein increased transduction efficiency at multiple transduction dilutions. For example, a change in titer of 2.3-2.6 was observed for vector pseudotyped with exemplary retronectin co-display fragment CH296_F and BaEVTR as compared to vector expressing only BaEVTR fusogen. Moreover as shown in FIG. 7D, BaEVTR fusogen and retronectin co-display improves infectivity on mobilized peripheral blood (mPB) CD34+ cells as compared with BaEVTR fusogen alone.

CD34+ editing titers are shown in FIG. 7E. Virus like particles comprising the variant BaEVTR and packaged with gag-Cas9 cargo and B2M gRNA as described above were concentrated 100× before being titered on CD34+ cells with vectofusin. B2M editing readout was assessed by flow cytometry 7 days post transfection. It was observed that combining co-display of retronectin with BaEVTR fusogen improved in vitro CD34 editing titer >10×.

### Example 3 Development and In Vivo Testing of Co-Display Constructs

Lentiviral vector (LV) was pseudotyped with a fusogen and a co-display protein as described below to generate a co-display lentiviral vector and used to assess frequency of transduced cells and transduction efficiency.

First, LV was pseudotyped with the variant BaEVTR fusogen targeting CD34 as well as a retronectin co-display peptide per the description in Example 2 (BaEVTR LV-CoD-CD34). Briefly, the HIV-derived lentiviral constructs were also engineered to include a cleavage site used by the MLV protease to cleave the cytoplasmic tail of the BaEVTR molecule, with the MACA cleavage site which is recognized by a HIV protease FIG. 8A. The BaEVTR LV were also equipped with an additional surface molecule (co-display) encompassing the VLA-4 and VLA-5 binding domains from the C-domain and CS-1 site respectively of the RetroNectin recombinant fibronectin fragment (FIG. 7F). This construct, called “Co-Display” (CoD), was added to the surface of the LV particle to see if it could enhance the BaEVTR LV-mediated gene transfer to HSPC via binding to the VLA-4 and VLA-5 integrin receptors (see FIG. 8B). As shown in FIG. 8C, in vitro measurements showed increasingly higher titers adding first the MACA cleavage site and then the CoD construct to our BaEVTR LV. In line with this observation, the resulting BaEVTR-CoD LV (which is defined from this point on as comprising both the MACA and the CoD modifications) showed in general higher potency in vitro on resting CD34+ cells vs the standard BaEVTR-LV construct.

BaEVTR-CoD-LV (FIG. 8B) was assessed against a standard BaEVTR LV (with no co-display) in a short-term murine humanization model for 16 weeks (FIG. 8D) in NBSGW mice (Jackson Labs strain Hu-NSG-NBSGW-CD34, 702662). As shown in FIG. 8E, the composition of the humanized BM had a predominance of M, LP and L cells and a further skewing towards M and L cells detected by week 8 until the end of the experiment at week 16.

As shown in FIGS. 8E-8J, the BaEVTR-CoD LV was observed to display a statistically significant higher access to BM huCD45+(3.4% vs 1.5%) as compared to the BaEVTR-LV. Confirming the in vitro results, upon 3× consecutive doses of a BaEVTR-LV or a BaEVTR-CoD LV both at 3.04E+08 TU/mouse/dose over a D7-D9 window post-humanization, a significantly higher percent of GFP+ cells was detected at week 16 in the BM huCD45+ cell compartment of the mice treated with the BaEVTR-MACA-CoD LV vs the standard BaEVTR LV (FIG. 8E). Interestingly, looking at the transduction levels in the BM HSPC population overtime we noticed that despite the BaEVTR-CoD LV treated group carried a higher percent of GFP+ cells at week 4 than the animals having received the BaEVTR-MACA-LV, by week 8 the two groups displayed a similar transduction diverging again by week 16 with a higher percent of transduced cells detected in the BaEVTR-MACA-CoD LV group in HSPC at the end of the study (FIG. 8F). As depicted in FIG. 8F a similar dynamic was also observed when looking at the percent of GFP+ cells in the BM fractions defined by the quadrants of FIG. 8G, particularly for what concerned the LP and L cell populations. Accordingly, the BaEVTR-CoD LV displayed higher long-term transduction levels in the PB not only in the whole huCD45+ cells or in the long-lived mature CD19+ lymphocytes but also in the CD13+CD33+ myeloid cell population, which is subjected to a higher turnover and therefore reflective of an active long-term stem cell output (FIG. 8H). These transduction levels were also confirmed molecularly by VCN evaluation (FIG. 8I) Lastly, similar percent of GFP+ cells were observed in the spleen huCD45+ cell progeny again showing superior long-term transduction using the BaEVTR-MACA-CoD LV (FIG. 8J). Importantly, histology performed on sternums collected from treated animals at different timepoints after infusion showed colocalization of human nuclei and GFP signals in proximity of sinusoidal vessels and in the endosteum, confirming the stable transduction by both vectors not only of committed progenitors but also of long-term hematopoietic stem cells residing in the endosteal niche (FIG. 8K). In summary, this represents a higher transduction efficiency in the HSPC population and in multiple BM compartments including primitive (P), myeloid (M), lymphoid progenitor (LP) and lymphoid (L) cells. Similar observations were made in the peripheral blood (PB) huCD45+, myeloid (CD13+CD33+) and lymphoid (CD19+) populations and in the Spleen, which was measured as a secondary organ for engraftment of mature human blood cells.

Overall, these data supported use of the MACA and CoD modification in combination with the BaEVTR envelope in a LV construct to enhance HSPC on-target in vitro and in vivo potency and persistence of gene modification.

In a second set of experiments, LVs were pseudotyped with one or more targeted fusogens, each targeting CD133 with a different scFv and generated along with a retronectin co-display peptide. The CD133-retargeted fusogen contained one or more exemplary anti-CD133 scFvs (e.g., two exemplary anti-CD133 scFVs) as a fusion to the C-terminus of the exemplary Nipah G (NiV-G) protein sequence GcA34 that was mutated to ablate its native tropism (Bender et al. 2016 PLoS Pathol 12(6):e1005641; set forth in SEQ ID NO:228). The lentiviral vectors also were pseudotyped with the exemplary Nipah F (NiV-F) protein sequence NivFdel22 (SEQ ID NO:226; or SEQ ID NO:227 without a signal sequence; Bender et al. 2016 PLoS). LV1 (NivG-CD133scFv-1) and LV2 (NivG-CD133scFv-2) were then combined to generate a mixed solution of LV (NiV LV-CD133).

In this example, a LV retargeted towards CD133 was assessed for in vivo potency and specificity. CD133 is not only highly expressed in CD34+ cells, but the difference in the membrane expression with respect to the CD34− cells is more accentuated as compared to what observed for other markers (FIG. 9B), both features that makes it a promising target for a HSPC-specific delivery system. Therefore, a NiV fusogen retargeted towards CD133 was designed and incorporated in a LV construct alone or in combination with a CoD (FIG. 9A).

As shown in FIG. 9A, assessing NiV LV-CD133 with or without retronectin co-display for transduction of resting CD34+ cells demonstrated a higher transduction efficiency than VSV-G LV and comparable with a BaEVTR-LV. At a MOI of 25, a NiV-CD133-LV was able to transduce specifically and only CD34+ cells in resting or stimulated conditions while differently from BaEVTR and VSV-G LVs could not transduce CD3+ T cells (FIG. 9B). Given the promising in vitro transduction performance, a first specificity test was performed in vitro comparing the ability of the NiV CD133 to transduce either cytokine activated or resting CD34+ cells (expected to express the CD133 target molecule) or CD3+ T cells (which are CD133−) and used the broadly tropic VSV-G LV and BaEVTR LV as controls. As shown in FIG. 9B, using our LVs at MOI 25 we could observe that the NiV-CD133 vector transduced efficiently CD34+ HSPC in both stimulated or resting condition while could not transduce T cells even upon cytokine activation differently from what observed when using a VSV-G or a BaEVTR-LV.

Further analysis of a full titration curve as shown in FIG. 9C demonstrated that the NiV-CD133-LV was able to transduce specifically and only CD34+ cells in resting or stimulated conditions that express CD133, but could not transduce CD3+ T cells that are negative for CD133 expression. When measuring the titers over a group of immortalized or primary cell lines, the NiV-CD133-LV was observed to have higher specificity for CD133+ cells as compared to VSV-G and BaEVTR (FIG. 9C). The NiV-CD133 LV were used to transduce HSPC or T cells in vitro at different dilutions, which confirmed that the specificity for CD133+ HSPC was maintained at all doses in either resting or stimulated conditions and with the presence of the CoD constructs (FIG. 9C). As a specificity “stress-test” for the ability of the NiV-CD133 construct to preferentially target CD133+ cells in vitro we next designed and conducted a full titration experiment against a variety of immortalized cell lines including a HEK cell line transiently overexpressing CD133 (HEK CD133 OE) again using VSV-G and BaEVTR LV as controls. As shown in heatmaps in FIG. 9D-9F, the NiV-CD133 displayed a specificity for the cell lines carrying higher membrane levels of the CD133 receptor (HEK CD133 OE and Caco2), a very different pattern as compared to what observed for VSV-G and BaEVTR LV which showed high level of transduction across the board irrespectively of the CD133 expression. Because immortalized cell lines differ substantially from their normal counterparts, we next extended our specificity test to a set of human primary cells, including endothelial cells, epithelial cells, and primary human hepatocytes (PHH), while adding to the comparison a NiV-CD133-CoD LV construct to assess any potential effect of the presence of the CoD on the specificity of the CD133 retargeted LV.

As displayed in FIG. 9E, this screening again confirmed the specificity of the NiV-CD133 construct to transduce CD133+CD34+ cells with respect to the other cell lines and differently from what observed when using VSV-G and BaEVTR LV constructs. Notably, the addition of the CoD not only preserved the specificity of the retargeted LV but also showed higher on-target titers. We noticed though that the NiV-CD133 showed some degree of transduction in certain endothelial and epithelial cells as well as in PHH. To better contextualized the transduction preferences observed with our LV constructs we performed a comprehensive RNA-seq analysis of immortalized and non-hematopoietic primary cell lines to investigate the expression levels of the known target receptors for VSV-G (LDLR) BaEVTR (ASCT1 and ASCT2) as well as the expression of CD133 and the CoD target molecules VLA-4 and VLA-5. As shown in FIG. 9F, most immortalized and primary cells displayed high levels of LDLR, ASCT1 and ASCT2 while CD133 expression was confined to Caco2 and Renal epithelial cells. Interestingly VLA-4 and VLA-5 showed distinct expression patterns and high expression in endothelial and epithelial cells, which nonetheless did not seem to impact the specificity of the NiV-CD133-CoD LV construct in primary cells. These data suggest that the NiV-CD133 titers measured in certain endothelial and epithelial cells could be due to GFP expression from episomal proviral species due to the exposure of non-proliferating adherent cells to artificially high LV doses, a setting that we envisage substantially deviates from a physiological in vivo scenario.

A group of LV generated with a BaEVTR or NiV retargeted fusogen and with or without retronectin codisplay was then assessed in the FRG mouse model to determine transduction in one of the exemplary off-target filter organs selected for study. When transplanted, the FRG mouse readily accepts a xenograft and mice are highly repopulated with human hepatocytes. Human hepatocytes were infused Day 0, followed by vector dosing on Day 84. Vector dosing was as follows: VSV-G=2.7e9 TU/mouse, BaEVTR LV=1.3e9 TU/mouse/dose, BaEVTR-CoD LV=1.3e9 TU/mouse/dose, NiV-CD133-LV=7.25e8 EU/mouse/dose, NiV-CD133-CoD-LV=2.02e7 EU/mouse/dose. As shown in FIG. 9G, both BaEVTR and NiV-CD133 pseudotyped vectors, with or without retronectin co-display, did not show a propensity to transduce human hepatocytes in vivo, while VSV-G LV or a NiV LV carrying a binder for the ASGR1 hepatocyte-specific molecule showed potent transduction of the human hepatocytes.

### Example 4 Development and Assessment of BaEVTR Co-Display Constructs

Virus like particles (VLP) was pseudotyped with a BaEV fusogen and retronectin co-display protein to generate a co-display lentiviral vector and used to assess lentiviral transduction and gene editing of resting HSCs.

In this example, VLP was pseudotyped with a BaEV fusogen containing modified cytoplasmic tail sequences (e.g., such as a protease site modifications) as set forth in the Table E8 below. VLP was produced with a viral chassis from either of MLV or HIV, such that the VLP comprised either MLV or HIV-based Gag and packaging components. These constructs were assessed for comparative titer on 293LX cells as shown in FIG. 10A. It was observed that crude VLPs showed the highest titers with MLV chassis.

TABLE E8
Exemplary BaEV Fusogens

| | | |
|---|---|---|
| BaEVTR | BaEV fusogen with T | SEQ ID NO: 795 |
| | (cytoplasmic) and R | |
| | (inhibitory) domains | |
| BaEVTRLess | R domain removed | SEQ ID NO: 796 |
| BaEVTR_MACA | Protease site | SEQ ID NO: 797 |
| | modification | |

Further experiments were conducted with BaEV fusogens to assay the impact of BaEV fusogen as well as guide RNA and co-display protein on editing titers. For co-display conditions, each BaEV fusogen was expressed with a retronectin peptide as described in Examples 2 and 3 and packaged with gag-Cas9 cargo and various B2M gRNAs at one of two ratios (e.g., ratio 1 or ratio 2). Once produced, VLPs were titered using human CD34+ cells. As shown in FIG. 10B., VLPs using a MLV chassis with BaEVTR_MACA, Guide 5, and retronectin codisplay show highest editing titers on CD34+ cells. These data taken together support that BaEVTR_MACA, retronectin co-display, and guide 5 combined to improve overall in vitro editing titers in CD34+ cells by more than 10×.

### Example 5 Development and Assessment of BaEVTR and CD133 Fusogens In Vivo

In this example, LV pseudotyped with a BaEVTR fusogen was assessed in vivo as compared with a retargeted NiV-G directed to CD133, NiV-CD133-LV described above. The BaEVTR fusogen was generated as a variant fusogen, wherein the protease site between the T (cytoplasmic) and R (inhibitory) domains was mutated (SEQ ID NO: 797). BaEV, which has high tropism to CD34+ cells, was packaged with gag-Cas9 cargo and B2M gRNA. The CD133 construct used for these experiments is as described in Example 3. Briefly, LVs were pseudotyped with one or more targeted fusogens, each targeting CD133 with a different scFv and generated along with a retronectin co-display peptide. The CD133-retargeted fusogen contained one or more exemplary anti-CD133 scFvs (e.g., two exemplary anti-CD133 scFVs) as a fusion to the C-terminus of the exemplary Nipah G (NiV-G) protein sequence GcA34 that was mutated to ablate its native tropism (Bender et al. 2016 PLoS Pathol 12(6):e1005641; set forth in SEQ ID NO:228). The lentiviral vectors also were pseudotyped with the exemplary Nipah F (NiV-F) protein sequence NivFdel22 (SEQ ID NO:226; or SEQ ID NO:227 without a signal sequence; Bender et al. 2016 PLoS). LV1 (NivG-CD133scFv-1) and LV2 (NivG-CD133scFv-2) were then combined to generate a mixed solution of LV (NiV LV-CD133).

For CD133, having shown on-target potency and specificity of the NiV-CD133 in vitro, this construct was tested in vivo in the NBSGW model, comparing its performance with a BaEVTR LV. We initially sought to test these LVs in the D7 post-humanization experimental model (FIG. 11A). It should be noted that, tracking the CD133 expression during the early phases of human hematopoietic reconstitution in these animals, we observed a rapid decline of this target receptor already at D2 after transplant and continuing until D12. This reflected not only a differentiation of the CD34+ cells in vivo (decreasing percent of CD133+ cells) but also a substantial general downregulation of the CD133 receptor in human HSPC (decreasing mean fluorescence intensity, MFI, in CD133+ cells) as compared the originally infused human CD34+ cell product (FIG. 11B). Therefore, the CD133+ population represents a “moving target” for measuring on-target transduction days after dosing, as well as puts the NiV-CD133 LV at a slight disadvantage as compared to the BaEVTR LV when it comes to specifically measuring in vivo on-target activity. Nonetheless, this work confirmed CD133 as a marker that stably identifies human HSPC in the BM of NBSGW mice throughout the study (FIG. 11C).

LV was assessed in a short-term NBSGW humanized mouse model. CD34+ human cells were infused into the mice on Day 0, followed by dosing of BaEVTR-LV or NiV-CD133-LV vector on Day 7 (FIG. 11A). Animals were dispatched on Day 12 for study. Vector dosing was comparable to that used with FRG models (VSV-G=2.7e9 TU/mouse, BaEVTR LV=1.3e9 TU/mouse/dose, NiV-CD133-LV=7.25e8 EU/mouse/dose). A second cell line-based titer determination mean: BaEVTR LV at 3.3E+08 TU/mouse and NiV-CD133 LV at two different concentrations 5.9 TU/mouse (300× concentrated) or 1.7E+08 TU/mouse (500× concentrated). As shown in FIGS. 11D-11G, vector comprising NiV-CD133 showed comparable access to huCD45+ cells as compared to BaEVTR-LV at equivalent dosing at 2 different vector concentrations. Moreover, NiV-CD133 LV enriched access to HSPC and CD133+ cells in the BM at D12. These results suggest higher specificity for CD133+CD34+ progenitors than BaEVTR. FIG. 11D shows comparable transduction efficiency of BM huCD45+ at D12 post-humanization (5 days after dosing) with the NiV-CD133 LV, at both concentrations, vs the BaEVTR LV. However when gating on BM HSPC (FIG. 11E) or BM CD133+ cells (FIG. 11F) we observed a significantly higher percent of GFP+ cells obtained with the NiV-CD133 LV 500x vs the BaEVTR LV. Notably, when gating on GFP+ cells both the 300× and 500× concentrated NiV-CD133 LV showed a significantly higher fraction of transduced cells having preserved the CD133 marker from the day of vector dosing (33%) as compared to what observed using a BaEVTR LV (13%) (FIG. 11G). These data suggest that the NiV-CD133 LV displays on-target potency and specificity for human BM HSPC in vivo in the D7-post humanization experimental model.

In view of the observed rapid changes in CD133 expression after infusion, the on-target potency of the NiV-CD133-CoD LV was tested in the Co-infusion experimental model where the surface expression of the CD133 marker in human CD34+ cells would still be at physiological levels. To create a more challenging scenario for measuring on-target specificity, in a group of mice we infused a mix of 5.0xE+05 CD34+ cells diluted in 1.0xE+06 CD133− human peripheral blood mononuclear cells (PBMC) from the same donor, immediately followed by LVs dosing. Transduction efficiency was measured at D12 comparing the results with the group of mice infused with 5.0xE+05 huCD34+ cells only (FIG. 11H). The mice dosed with CD34+PBMC showed higher human PB engraftment as well as the presence of a substantially higher fraction of mature PB blood cells including CD3+ T cells at D7 and D12 after infusion acting as potential off-target cell population vs the mice dosed with CD34+ cells only (FIG. 11I-11J). When gating, at the end of the study, on BM huCD45+ cells, we observed a comparable frequency of GFP+ cells in all groups, upon dosing BaEVTR LV or NiV-CD133-CoD LV at 3.0E+08 TU/mouse and 7.8E+07 TU/mouse respectively (FIG. 11K).

Finally, the ability of BaEVTR LV and NiV-CD133-CoD-LV to transduce target cells in the presence of off-target cellular sinks was determined. Here, animals were infused with 500,000 human CD34+ cells with or without the addition of 10 million (10M) donor-matched PBMCs on Day 0, along with administration of vector as described. As shown in FIG. 11L-11Q, NiV-CD133-CoD LV showed higher specificity for HSPC, CD34+ Primitive (P) and Lymphoid Progenitors (LP), and CD133+ cells, as well as lower propensity to target mature PB cells as compared to BaEVTR LV. Interestingly, the BaEVTR LV showed a general trend towards higher transduction levels in the CD34+PBMC infused group (24%), as compared to the NiV-CD133-CoD LV treated mice (19%), although this difference was not significant. Conversely, when gating on human BM HSPC we observed a significantly higher transduction in the CD34+PBMC infused group dosed with the NiV-CD133-CoD LV (20%) vs the BaEVTR LV (14%) (FIG. 11L). In the animals infused with the CD34+PBMC mix, this was mirrored by a higher fraction of GFP+ cells observed in the BM CD34+P and LP cell fractions when dosing the NiV-CD133-CoD LV vs what observed using the BaEVTR LV, which instead displayed higher transduction in the more mature M and L cell populations (FIG. 11M and FIG. 11N). On the same line, the NiV-CD133-CoD LV showed higher specificity for BM CD133+ cells in both experimental groups, regardless of the presence of human PBMC at the time of dosing, as compared to BaEVTR LV (FIG. 11O and FIG. 11P). Furthermore, as shown in FIG. 11Q, the NiV-CD133-CoD LV displayed significantly lower transduction of mature PB cells at D12 as compared to the BaEVTR LV when dosed in NBSGW immediately after the infusion of the CD34+PBMC mix. Overall, these data showcased the higher in vivo specificity of the NiV-CD133-CoD construct for human HSPC with respect to a broadly tropic vector.

As described above, both the BaEVTR and the NiV-CD133 LVs transduced PHH in vitro. Should the human liver act as a “sponge” upon systemic delivery, it could substantially decrease on-target bioavailability of these vector particles and increase the chances of hepatotoxicity. This eventuality cannot be measured in the NBSGW mouse, because as other murine cells, murine hepatocytes are refractory to host transduction by these vector constructs. To address this issue, we therefore exploited the Fah^{(-/-)}Rag-2^{(-/-)}Il2rg^{(-/-)} (FRG) mouse model which can host a liver humanized with PHH.

At 84 days after liver humanization we injected in these FRG mice 3 IV doses of a BaEVTR LV (1.3xE+09 TU/mouse/dose), a BaEVTR-CoD LV (1.3xE+09 TU/mouse/dose), a NiV-CD133-LV (7.3xE+08 TU/mouse/dose) and a NiV-CD133-CoD LV (2.2xE+07 TU/mouse/dose) resembling the dosing schedule and levels used in the NBSGW mice (FIG. 11R). As positive control, we dosed a group of animals with a single dose of a VSV-G LV (2.7xE+09 TU/mouse/dose) which is known to transduce human hepatocytes. Remarkably and differently from what observed in vitro, negligible transduction of both human hepatocytes and mouse non-parenchymal cells (NPC, which include Kupffer cells) was observed in the humanized liver of the FRG mice 14 days after treatment with either the BaEVTR or the NiV-CD133 LVs with or without CoD, as compared to the transduction observed in the VSV-G LV treated group, suggesting in vivo avoidance by both our LVs of this relevant off-target cellular “sink” (FIG. 11S). These data were confirmed upon liver histology for measuring colocalization of human nuclei and GFP signal (FIG. 11T). To explain the stark difference with the in vitro titration experiment we hypothesized that PHH could change in nature upon thawing and in vitro culture prior to vector exposure. To test this hypothesis, we generated RNA-seq data comparing freshly thawed PHH, PHH cultured in vitro for 24 h (time of vector dosing for the in vitro titration experiments) and human hepatocytes freshly isolated from FRG mice prior to vector dosing. The cultured PHH show substantial differences in the expression of a series of transcripts as compared to the freshly thawed PHH and to the human hepatocytes isolated in vivo from FRG mice (FIG. 11U). Notably, the ASCT2 transcript, the product of which is used by BaEVTR for fusion to the cellular membrane, is artificially upregulated already after 24 h in the culture media with respect to the baseline expression in freshly isolated PHH (FIG. 11V). Overall, these data suggest that the in vivo FRG mouse setting might indeed constitute a more physiological scenario to test the tropism of different envelopes towards human hepatocytes than the in vitro transduction of PHH.

### Example 6 Design and Assessment of In Vitro and In Vivo Potency of a BaEVTR VLP Delivering Genome Editing Payloads to Human HSPC

The results obtained with the LV constructs, suggest that it is possible to efficiently engineer human HSPC when in their natural state and, upon systemic delivery in vivo, access them directly in the BM niche, while at the same time avoiding main off-target cellular compartments such as the liver. These data provided a solid ground for developing potent VLPs for delivering editing payloads to human HSPC. To this aim, a VLP was generated carrying a Beta 2 Microglobulin (B2M) guide RNA (see Extended data Table 1), a CRISPR-Cas9 editor covalently fused with a MLV capsid and a BaEVTR envelope (FIG. 12A).

| Target info | | | | |
|---|---|---|---|---|
| Target | Target Position | Target | | Editor |
| Locus | (GRCh38/hg38) | Strand | Target Sequence | Type |
| B2M | chr15:44,711,545-44,711,564 | − | GGCCACGGAGCGAGACATCT | Cas9 |
| B2M | chr15:44,711,566-44,711,585 | − | GAGTAGCGCGAGCACAGCTA | Cas9 |
| BCL11a | chr2:60,495,252-60,495,271 | − | TTTATCACAGGCTCCAGGAA | ABE |
| HBG1/2 | chr11:5,254,880-5,254,899 | + | CTTGTCAAGGCTATTGGTCA | Cas9 |

The B2M locus was selected because it provides a surrogate FLOW cytometry readout for gene editing, as exemplified in FIG. 12B. Using a protocol described in FIG. 12C, the efficiency of the BaEVTR VLP was tested in vitro on resting CD34+ cells. We observed up to 75% B2M− cells as measured by FLOW corresponding to up to 93% of edited alleles as measured by sequencing of the B2M locus (on target analysis, OTA) (FIG. 12C and Extended data Table 2).

| | OTA primers (include an overhang sequence | |
|---|---|---|
| Target | in grey to accommodate illumina adaptors) | |
| Locus | Fwd primer | Rev primer |
| B2M | CTCTTTCCCTACAC | CTGGAGTTCAGACG |
| | GACGCTCTTCCGAT | TGTGCTCTTCCGAT |
| | CTATATAAGTGGAG | CTGGAGAAGGGAAG |
| | GCGTCGCG | TCACGGAG |
| BCL11a | TATGAGGGTGACCT | ATGGGGAGAGAAGA |
| | CTGGATCC | GTGGAAAC |
| | (1st PCR) | (1st PCR) |
| | CTCTTTCCCTACAC | CTGGAGTTCAGACG |
| | GACGCTCTTCCGAT | TGTGCTCTTCCGAT |
| | CTTCCTCTTCTACC | CTGGAAGGCAGCTA |
| | CCACCCAC | GACAGGAC |
| | (2nd PCR) | (2nd PCR) |
| HBG1/2 | CTCTTTCCCTACAC | CTGGAGTTCAGACG |
| | GACGCTCTTCCGAT | TGTGCTCTTCCGAT |
| | CTCCTGGCCTCACT | CTGAATCGGAACAA |
| | GGATACTCTA | GGCAAAGGC |

To test the ability to edit individual HSPC subtypes at a low dose, 5 HSPC fractions were FACS-sorted, as done for the LV constructs, and exposed individually in resting conditions to the BaEVTR VLP at an MOI of 16 or an MOI of 83. As shown in FIG. 12D, the VLP is capable of efficiently editing all HSPC subtypes including HSC (6.7% and 38% editing at MOI 16 and 28% and 61% at MOI 83 as measured by FLOW and OTA respectively). Although useful as a real-time readout, the FLOW cytometry assessment of B2M− cells underestimated the editing efficiency of the VLP, particularly in the more quiescent HSPC subtypes like HSC and MPP. This could be due to the stringency of our gating strategy, capturing only cells with biallelic edits resulting in complete B2M KO, combined with the fact that the B2M turnover tends to be slower in the most primitive progenitors even upon addition of cytokines to the media on Day 2 after VLP removal. When measuring the relative proportion of editing variants in each HSPC subtype as well as in the bulk HSPC population, it was observed that the predominant fraction is constituted by deletions, except for the GMP fraction which hosted a substantially higher number of insertion events as compared to the other subpopulations, suggesting a proneness of more committed progenitors to accommodate these types of editing variants (FIG. 12D.

Based on these promising in vitro results, a first in vivo test was performed in the D7 post-humanization experimental model, injecting IV 1 or 3 doses of our BaEVTR VLP at 5.9E+08 EU (Editing Units)/mouse/dose in combination with either 1 or 3 consecutive AMD3100 administrations, measuring editing in the BM 5 days after the last dosing (FIG. 12E). As shown in FIG. 12F, we observed a dose-dependent high editing efficiency in huCD45+ in the BM achieving 20% and 47% B2M-cells as measured by FLOW with 1 or 3 doses respectively, mirrored by 16% and 31% B2M− cells in the human HSPC fraction (FIG. 12G). Interestingly, when looking at individual HSPC subtypes the editing efficiency of BaEVTR VLP was not always dose dependent as exemplified by the 24-fold higher editing in HSC at 3 doses vs 1 dose (12% vs 0.5% B2M− cells respectively) or the similar access observed at both doses in the MLP or PreBNK fractions (FIG. 12G). This could be a due to a VLP dose-threshold effect combined with an improved access to primitive subtypes when AMD3100 is administered for 3 consecutive days. When measuring editing efficiency by OTA we observed an even higher access to huCD45+ cells in the BM as compared to FLOW, reaching 47% and 71% edited alleles with 1 dose and 3 doses respectively (FIG. 12H). Of note, the distribution of editing variants was different as compared to what observed in vitro, showing a more balanced representation of insertions and deletions as well as a similar profile when comparing 1 vs 3 doses (FIG. 12T).

To test the performance of the BaEVTR VLP in a limiting scenario with a higher number of human target cells, we injected a single dose of the same VLP (5.9E+08 EU/mouse) in NBSGW mice at 9 weeks post humanization (FIG. 12U). Even if, in this experimental setting 85% of the BM was composed by huCD45+ cells (FIG. 12V), the level of B2M editing achieved in BM huCD45+ cells or HSPC after a single IV dose of our BaEVTR VLP was comparable to the one achieved with 1 dose delivered at D7 post-humanization ((FIG. 12W). This suggests that, with the employed dosing regimen, the number of human target cells in the BM did not influence the in vivo potency of the BaEVTR VLP and confirms that the D7 post-humanization model represent an adequate scenario to measure in vivo access to human cells in the BM.

The short-term in vivo readout showcased the potential of the BaEVTR VLP platform to reach high numbers of human BM cells upon systemic in vivo delivery. To confirm permanent editing of human hematopoietic cells and access to long-term human HSC, a long-term follow up study was conducted, similarly to what done for testing the LV constructs, with interim BM takedowns at week 4 and week 8 and terminal readout at week 16 after 3 VLP dosing at D7-D9 post humanization (FIG. 12I). Firstly, measuring B2M− cells by FLOW in the huCD45+ cell fraction at week 16 confirmed that 6.4% edited cells could still be detected in the BM of the treated animals several weeks after dosing (FIG. 12J). Looking at the dynamics of editing in the BM HSPC fraction overtime, we noticed a substantial drop in the percent of B2M cells from week 4 to week 8. One hypothesis for this drop is that it was due to the exhaustion of edited short-living progenitors, later resulting in a remarkably stable editing over the following 8 weeks until the end of the study at week 16 when, by FLOW, we could still measure 7.7% of B2M edited BM HSPC (FIG. 12K). This was mirrored by similar editing levels in multiple HSPC subtyped including the HSC/MPP population as shown in FIG. 12L. Looking at the editing levels in the 4 main human BM fractions overtime, it was observed that the largest drop in B2M− cells between week 4 and week 8 was indeed observed in the L and LP fractions (FIG. 12M) representing the most active progenitors during this phase of human cell colonization of the mouse BM, therefore also the ones more likely to undergo higher rates of turnover and exhaustion. As shown in FIG. 12N, comparable dynamics in the mature human PB progeny overtime were observed with final readouts at week 16 showing an average of 3.5%, 2.5% and 3.9% B2M− cells in the huCD45+, huCD13+CD33+a huCD19+ fractions respectively, with the spleen showing similar level of HSPC edited cells at week 16 as compared to the BM (FIG. 12O). Because the FLOW readout tends to underestimate the level of B2M editing, a thorough analysis of molecular editing at multiple timepoints was performed by OTA. The results displayed in FIG. 12P showed a contrast with the B2M− cell fraction measured by FLOW in the PB, whereby the editing efficiency measured by OTA at week 4 was 71% (vs the 45% measured by FLOW) and at week 16 the percent of edited alleles was still in the double digits (27% vs the 3.5% measured by FLOW) (FIG. 12P). Similar results were observed from OTA conducted on the 4 BM fractions reflecting the dynamics measured by FLOW but highlighting higher levels of editing, with a sharper drop in edited cells observed in the LP and L fractions going from 85% and 91% to 31 and 30% at week 16 respectively (FIG. 12Q). Notably this was mirrored by the preservation of 28% edited alleles in the least differentiated multipotent P fraction, which overall resulted in the maintenance of 29% editing of the total huCD45+ cell population measured by OTA at the end of the study vs the 6.4% originally measured by FLOW (FIG. 12R). The large discrepancy between FLOW and OTA data, suggested that the most differentiated cells could carry monoallelic edits or editing variants whose effect on B2M expression was not easily identifiable by FLOW and supports the hypothesis that a full knock-out of the B2M locus could have a detrimental effect upon cell differentiation of human long-term progenitors. Looking at the selection of editing variants in PB cells overtime, we noticed that while the percent of deletions remained relatively stable overtime (21% at week 4 vs 17% at week 16%) the relative fraction of insertions dropped from 43% at week 4 to 7.4% at the end of the study (FIG. 12S).

Notably, the LP and L fractions carried a largely predominant percent of insertions events early on upon in vivo editing (similarly to what observed above upon editing of the more committed GMP in vitro), later dropping to levels comparable to the other BM populations which reflected the stabilization of the BM output driven by long-term primitive progenitors (FIG. 12X). This suggests that editing variants occur at different frequencies in human HSPC at different states of activation/differentiation and that given B2M locus disruptions might be negatively selected in vivo in the human cells repopulating the NBSGW mouse model.

Overall, these data support the notion that the BaEVTR VLP construct displays editing potency in resting human HSPC in vitro as well as access in vivo to long-term BM HSPC with preservation of double digits editing levels in the human cell progeny for at least 16 weeks after vector dosing.

### Example 7 In Vivo Potency of Enhanced VLP Constructs and Delivering of Therapeutic Genome Editing Payloads to Human HSPC

Having achieved high and sustained levels of gene editing in vivo in human HSPC with our BaEVTR VLP delivery vector, LVPs were modified to enhance either specificity or potency using the same or similar modifications as done for the LV constructs. VLPs were generated carrying a NiV-CD133 retargeted fusogen and with or without a co-display and with either a MLV or an HIV capsid. All NiV-CD133 VLPs achieved editing in resting CD34+ in vitro although their titers were lower than what observed with the BaEVTR VLP design (FIG. 13A). The VLP showing the highest editing potency at lower dilutions was the NiV-CD133-CoD VLP combined with a HIV gag. This VLP was put forward for testing in vivo in the co-infusion model (FIG. 13B). Co-infusion model is as described in International Patent Application No. PCT/US2023/076747, which is hereby incorporated by reference in its entirety. As shown in FIG. 13C, similar levels of editing with one dose of BaEVTR VLP (at 1.8E+07 EU/mouse) or NiV-CD133-CoD VLP (at 5.7E+06 EU/mouse) in PB huCD45+ cells over time up to week 8 after cell and vector infusion. While we did not observe significant differences in the percent of PB edited huCD45+ cells at the end of the study, in the BM we detected an almost 2-fold higher editing with the BaEVTR VLP formulation (FIG. 13D). However, when gating on the few huCD133+ cells detectable in the BM at week 8 after humanization the percent of edited cells was comparable in the two treated groups (FIG. 13D). This was the result of a significantly higher enrichment of huCD133+ cells within the B2M-cells generated with the NiV-CD133-CoD VLP vs the BaEVTR VLP (FIG. 13E), suggesting again that the CD133 retargeted fusogen displays a higher specificity for CD133+ cells as compared to BaEVTR. Measuring the editing efficiency by OTA the FLOW readout tends to underestimate the actual editing levels obtained over time with the two VLPs (FIG. 13F). The OTA data obtained on BM huCD45+ cells at week 8 confirmed that the two constructs reached a comparable editing efficiency (5.6% with the BaEVTR VLP vs 5.5% with the NiV-CD133-CoD VLP, FIG. 13G).

Our BaEVTR vectors showed high potency on human HSPC coupled with the potential avoidance of major in vivo cellular off-targets such as human hepatocytes. We therefore focused our efforts on improving the BaEVTR VLP design, starting from assessing whether equipping a BaEVTR VLP with a CoD would result in enhancement of on-target in vitro and in vivo potency similar to those observed for the LV constructs. To this aim, a BaEVTR-CoD VLP was designed and produced carrying the MACA HIV protease cleavage site, a MLVgag-Cas9 fusion protein and a B2M gRNA editing payload, as schematized in FIG. 13A, and tested it in vitro on resting human CD34+ cells. The data reported in the FIG. 13H, show progressively higher titers in vitro in resting HSPC adding the MACA cleavage site and the CoD as compared to the original BaEVTR VLP construct, similarly to what was shown for the LV constructs. The side-by-side analysis of two BaEVTR VLP carrying the MACA cleavage site with or without CoD displayed on FIG. 13I, showcase the beneficial effect of the addition of the CoD on improving editing potency in resting HSPC.

These results set the basis for conducting an in vivo study to establish on-target potency and stability of gene editing, using the D7 post-humanization model and following up mice for 8 weeks after 3 consecutive IV doses of a BaEVTR VLP carrying the MACA cleavage site plus (1.1E+08 EU/mouse/dose) or minus (1.8E+07 EU/mouse/dose) CoD at day 7-9 post-humanization (FIG. 13J). D7 post-humanization model is as described in International Patent Application No. PCT/US2023/076747, which is hereby incorporated by reference in its entirety.

In line with the results obtained in vitro, a higher percent of B2M− cells was measured by FLOW on BM huCD45+ cells at week 8 in the group treated with the BaEVTR-CoD VLP vs the BaEVTR VLP design (FIG. 14A and FIG. 14B). A similar trend was observed when looking at the B2M− cell fraction in the BM HSPC compartment (FIG. 14C) and in all BM fractions (FIG. 14D) as well as by looking at the B2M− fraction over time in all main PB populations (FIG. 14E) and in the BM progeny found in the Spleen at the day of takedown (FIG. 14F). The superior in vivo performance of the BaEVTR-CoD VLP construct was confirmed when measuring the levels of molecular edits by OTA in the PB over time (FIG. 14G) and in the BM at takedown (FIG. 14H, FIG. 14I) as well as showed that the introduction of the CoD did not alter the in vivo detection of the editing variants observed in the B2M locus with respect to what was observed with the BaEVTR VLP design (FIG. 14J).

The level and stability of on-target genetic engineering observed in vivo, together with the potential avoidance of cellular off-targets of high relevance such as human hepatocytes, support the potential employment of a BaEVTR-based vector for achieving in vivo gene correction of human HSPC. To explore this opportunity and to test the modularity and flexibility of our platform, we lastly designed two BaEVTR-CoD VLP constructs with two different types of editors carrying gene editing payloads aimed at targeting loci of therapeutic relevance for treating human hemoglobinopathies (FIG. 15A and FIG. 15B). One BaEVTR-CoD VLP was designed to carry a cargo composed by an ABE8e⁵⁶ Adenine Base Editor (ABE) fused with the MLV-gag protein and gRNA for editing the GATA-1 binding site of the human BCL11A gene, while a second vector was produced to deliver a CRISPR-Cas9 editor and a gRNA for disrupting the BCL11A binding site in the HBG1/2 promoter region. The guide RNAs were designed as described in FIG. 15B and Extended data Table 1. Upon in vitro titration in resting CD34+ cells we confirmed by OTA the ability of these two cargos to either induce a A-T to G-C conversion in the GATA binding site of the BCL11A locus (titer of 4.4E+05 with 82% editing at top dose) or to disrupt the BCL11A binding site of the HGB1/2 locus (titer of 4.9E+04 with 26% editing at top dose), although the efficiency of editing was lower than the one achieved with an identical construct targeting the B2M locus. We next tested these VLPs in the D7 co-infusion model (BCL11A-ABE VLP at 1.8E+09 EU/mouse/dose and HBG1/2-Cas9 VLP at 3.6E+08 EU/mouse/dose) and measured by OTA the editing efficiencies in the human BM fraction at the two loci 5 days after vector dosing (see FIG. 15C). As shown in FIG. 15D, we obtained in BM huCD45+ cells 26% A-T>G-C base conversion in the BCL11A target site and 7.5% editing of the HBG1/2 locus. When looking in details at the editing variants obtained with the ABE cargo, we could observe 59% conversion to G-C of both target A-T and 40% of the A-T immediately downstream of the GATA binding site (FIG. 15E, left panel). On the other hand, the relative contribution of the editing variants introduced by the CRISPR-Cas9 editor in the HBG1/2 locus was in line with the one observed for the B2M gene (FIG. 15E, right panel). Lastly, we wanted to assess whether we could measure the effect of our edits on inducing fetal Hemoglobin (HbF) upon erythroid differentiation of the gene edited HSPC. To this goal we isolated CD34+ cells from the BM of the mice dosed in vivo with our VLP and performed a colony assay in a semi-solid media containing cytokines promoting erythroid differentiation. As shown in the FIG. 15F we could observe similar proportions of CFU-GM and BFU-E colonies in all treated animals as compared to the untreated group. However, when looking at the HbF expression in our colonies by FLOW we detected a higher percent of HbF+ cells and a higher HbF MFI in the mice dosed with our VLPs, as compared to the untreated animals (used as controls for the expected background HbF induction resulting from this in vitro assay) (FIG. 15G, FIG. 15H)).

Overall, these data confirmed that the BaEVTR-CoD VLP can be equipped with different editors and gRNAs and can deliver these payloads in vivo to human HSPC achieving efficient editing of loci of therapeutic relevance.

### Example 8 NHP Prophetic Including the Lead Constructs

This example sets forth an exemplary design to assess various fusogen-payload combinations for administration in a Non-Human Primate (NHP). Virus-like particles (VLPs) are produced with a fusogen composed of a mutant Nipah virus G (NiV-G) fusogen (e.g., SEQ ID NO:228) in which the fusogen is retargeted to HSPC with a specific binder, such as a binder against CD133 or BaEVTR. The VLPs also include a retronectin co-display protein (e.g., any of the retronectin co-display proteins described herein) or a CD3 co-display protein (e.g., any of the CD3 co-display proteins described herein) on the lipid enveloped. The VLPs that are pseudotyped with the retargeted fusogens can also be pseudotyped with a blinded NiV-G that comprise point mutations E501A, W504A, Q530A and E533A to ablate binding to Ephrin B2/B3 (e.g. set forth in SEQ ID NO:228) but that is not retargeted by fusion with a binder sequence. For NiV fusogens, VLPs are produced with the Nipah F protein and the binder-retargeted Nipah G protein (see US 2019/0144885, incorporated by reference herein, e.g. NivFdel22 (SEQ ID NO:226; or SEQ ID NO:227, which does not have a signal sequence; Bender et al. 2016 PLoS) expressed on the lipid envelope. An exemplary NiV-G fusogen is each construct is the exemplary NiV-G sequence GcA34 (Bender et al. 2016 PLoS Pathol 12(6):e1005641; set forth in SEQ ID NO:228).

The VLPs are also packaged with a gene editing payload. Following VLP production, the VLPs are purified by standard downstream methods, such as by centrifugation in which the cell culture is centrifuged to pellet the cells, density-gradient separation and/or filtration, and the supernatant containing crude virus is collected.

A primary fusogen is selected based in part on overall VLP performance as measured by percent of long-term editing (i.e., edits resulting from the payload) in peripheral blood myeloid cells. A second fusogen is selected based in part on HSPC specific tropism as measured, for example, by percent of editing (i.e., edits resulting from the payload) in the various mature hematopoietic lineages derived from the HSPCs or in other cell lines vs the target HSPC population.

A payload (e.g., such as nuclease-based editor or base editing-based editor) is selected based on performance in resting NHP CD34+ cells in vitro as per OTA PCR based analysis. Performance can be either reporter-based or a functional readout (such as change in mRNA or protein levels in a biological sample). A genomic target is selected that is characterized by a “neutral” locus or a “therapeutic” locus homologous to a potential therapeutic target for a disease indication (e.g. BCL11A).

A model study design is illustrated in FIG. 16 NHP animals are first subject to a mobilization regimen as described above. Briefly, AMD3100 is administered for 3 consecutive days wherein VLP is administered roughly 2 hours after each mobilization at a controlled rate dosing. Without wishing to be bound by theory, mobilization of cells from the bone marrow to the peripheral blood is generally at its peak 1.5-3 hours post AMD treatment. Peripheral blood is collected throughout the study period, while bone marrow is collected through aspirates bi-monthly. Additional needle aspirates of the liver are performed to assess off-target editing as a function of safety and biodistribution. Animals are sacrificed at 9 months post vector dose for assessing permanent gene modification of the long-term HSC in the bone marrow and potential editing in off-target tissues.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ | | |
|---|---|---|
| ID NO | SEQUENCE | ANNOTATION |
| 1 | MPAENKKVRFENTTSDKGKIPSKVIKSYYGTMDIKKINEGLLD | NiVG protein |
| | SKILSAFNTVIALLGSIVIIVMNIMIIQNYTRSTDNQAVIKDALQ | attachment |
| | GIQQQIKGLADKIGTEIGPKVSLIDTSSTITIPANIGLLGSKISQST | glycoprotein (602 aa) |
| | ASINENVNEKCKFTLPPLKIHECNISCPNPLPFREYRPQTEGVS | |
| | NLVGLPNNICLQKTSNQILKPKLISYTLPVVGQSGTCITDPLLA | |
| | MDEGYFAYSHLERIGSCSRGVSKQRIIGVGEVLDRGDEVPSLF | |
| | MTNVWTPPNPNTVYHCSAVYNNEFYYVLCAVSTVGDPILNST | |
| | YWSGSLMMTRLAVKPKSNGGGYNQHQLALRSIEKGRYDKV | |
| | MPYGPSGIKQGDTLYFPAVGFLVRTEFKYNDSNCPITKCQYSK | |
| | PENCRLSMGIRPNSHYILRSGLLKYNLSDGENPKVVFIEISDQR | |
| | LSIGSPSKIYDSLGQPVFYQASFSWDTMIKFGDVLTVNPLVVN | |
| | WRNNTVISRPGQSQCPRFNTCPEICWEGVYNDAFLIDRINWIS | |
| | AGVFLDSNQTAENPVFTVFKDNEILYRAQLASEDTNAQKTITN | |
| | CFLLKNKIWCISLVEIYDTGDNVIRPKLFAVKIPEQCT | |
| 2 | ILSAFNTVIALLGSIVIIVMNIMIIQNYTRSTDNQAVIKDALQGI | NiV-G backbone ΔCT |
| | QQQIKGLADKIGTEIGPKVSLIDTSSTITIPANIGLLGSKISQSTA | |
| | SINENVNEKCKFTLPPLKIHECNISCPNPLPFREYRPQTEGVSNL | |
| | VGLPNNICLQKTSNQILKPKLISYTLPVVGQSGTCITDPLLAMD | |
| | EGYFAYSHLERIGSCSRGVSKQRIIGVGEVLDRGDEVPSLFMT | |
| | NVWTPPNPNTVYHCSAVYNNEFYYVLCAVSTVGDPILNSTY | |
| | WSGSLMMTRLAVKPKSNGGGYNQHQLALRSIEKGRYDKVM | |
| | PYGPSGIKQGDTLYFPAVGFLVRTEFKYNDSNCPITKCQYSKP | |
| | ENCRLSMGIRPNSHYILRSGLLKYNLSDGENPKVVFIEISDQRL | |
| | SIGSPSKIYDSLGQPVFYQASFSWDTMIKFGDVLTVNPLVVNW | |
| | RNNTVISRPGQSQCPRFNTCPEICWEGVYNDAFLIDRINWISAG | |
| | VFLDSNQTAENPVFTVFKDNEILYRAQLASEDTNAQKTITNCF | |
| | LLKNKIWCISLVEIYDTGDNVIRPKLFAVKIPEQCT | |
| 3 | ILSAFNTVIALLGSIVIIVMNIMIIQNYTRSTDNQAVIKDALQGI | NiV-G backbone ΔCT |
| | QQQIKGLADKIGTEIGPKVSLIDTSSTITIPANIGLLGSKISQSTA | and mutated |
| | SINENVNEKCKFTLPPLKIHECNISCPNPLPFREYRPQTEGVSNL | |
| | VGLPNNICLQKTSNQILKPKLISYTLPVVGQSGTCITDPLLAMD | |
| | EGYFAYSHLERIGSCSRGVSKQRIIGVGEVLDRGDEVPSLFMT | |
| | NVWTPPNPNTVYHCSAVYNNEFYYVLCAVSTVGDPILNSTY | |
| | WSGSLMMTRLAVKPKSNGGGYNQHQLALRSIEKGRYDKVM | |
| | PYGPSGIKQGDTLYFPAVGFLVRTEFKYNDSNCPITKCQYSKP | |
| | ENCRLSMGIRPNSHYILRSGLLKYNLSDGENPKVVFIEISDQRL | |
| | SIGSPSKIYDSLGQPVFYQASFSWDTMIKFGDVLTVNPLVVNW | |
| | RNNTVISRPGQSQCPRFNTCPAICAEGVYNDAFLIDRINWISAG | |
| | VFLDSNATAANPVFTVFKDNEILYRAQLASEDTNAQKTITNCF | |
| | LLKNKIWCISLVEIYDTGDNVIRPKLFAVKIPEQCT | |
| 4 | MPAENKKVRFENTTSDKGKIPSKVIKSYYGTMDIKKINEGLLD | NivG CT_45_FL |
| | SK | |
| 5 | MPAENKKVRFENTTSDKGKIPSKVIKSYYGTMDIKKINEGLLD | NiVG with NiVG |
| | SKILSAFNTVIALLGSIVIIVMNIMIIQNYTRSTDNQAVIKDALQ | CT_45_FL, mutated |
| | GIQQQIKGLADKIGTEIGPKVSLIDTSSTITIPANIGLLGSKISQST | (E501 A, W504A, |
| | ASINENVNEKCKFTLPPLKIHECNISCPNPLPFREYRPQTEGVS | Q530A, E533A) |
| | NLVGLPNNICLQKTSNQILKPKLISYTLPVVGQSGTCITDPLLA | |
| | MDEGYFAYSHLERIGSCSRGVSKQRIIGVGEVLDRGDEVPSLF | |
| | MTNVWTPPNPNTVYHCSAVYNNEFYYVLCAVSTVGDPILNST | |
| | YWSGSLMMTRLAVKPKSNGGGYNQHQLALRSIEKGRYDKV | |
| | MPYGPSGIKQGDTLYFPAVGFLVRTEFKYNDSNCPITKCQYSK | |
| | PENCRLSMGIRPNSHYILRSGLLKYNLSDGENPKVVFIEISDQR | |
| | LSIGSPSKIYDSLGQPVFYQASFSWDTMIKFGDVLTVNPLVVN | |
| | WRNNTVISRPGQSQCPRFNTCPAICAEGVYNDAFLIDRINWIS | |
| | AGVFLDSNATAANPVFTVFKDNEILYRAQLASEDTNAQKTIT | |
| | NCFLLKNKIWCISLVEIYDTGDNVIRPKLFAVKIPEQCT | |
| 6 | MLDSK | NivG_CT_05 |
| | | cytoplasmic tail |
| 7 | MLLDSK | NivG_CT_06 |
| | | cytoplasmic tail |
| 8 | MGLLDSK | NivG_CT_07 |
| | | cytoplasmic tail |
| 9 | MEGLLDSK | NivG_CT_08 |
| | | cytoplasmic tail |
| 10 | MNEGLLDSK | NivG_CT_09 |
| | | cytoplasmic tail |
| 11 | MINEGLLDSK | NivG_CT_10 |
| | | cytoplasmic tail |
| 12 | MKINEGLLDSK | NivG_CT_11 |
| | | cytoplasmic tail |
| 13 | MIKKINEGLLDSK | NivG_CT_13 |
| | | cytoplasmic tail |
| 14 | MDIKKINEGLLDSK | NivG_CT_14 |
| | | cytoplasmic tail |
| 15 | MMDIKKINEGLLDSK | NivG_CT_15 |
| | | cytoplasmic tail |
| 16 | MTMDIKKINEGLLDSK | NivG_CT_16 |
| | | cytoplasmic tail |
| 17 | MGTMDIKKINEGLLDSK | NivG_CT_17 |
| | | cytoplasmic tail |
| 18 | MYGTMDIKKINEGLLDSK | NivG_CT_18 |
| | | cytoplasmic tail |
| 19 | MYYGTMDIKKINEGLLDSK | NivG_CT_19 |
| | | cytoplasmic tail |
| 20 | MSYYGTMDIKKINEGLLDSK | NivG_CT_20 |
| | | cytoplasmic tail |
| 21 | MKSYYGTMDIKKINEGLLDSK | NivG_CT_21 |
| | | cytoplasmic tail |
| 22 | MIKSYYGTMDIKKINEGLLDSK | NivG_CT_22 |
| | | cytoplasmic tail |
| 23 | MVIKSYYGTMDIKKINEGLLDSK | NivG_CT_23 |
| | | cytoplasmic tail |
| 24 | MKVIKSYYGTMDIKKINEGLLDSK | NivG_CT_24 |
| | | cytoplasmic tail |
| 25 | MSKVIKSYYGTMDIKKINEGLLDSK | NivG_CT_25 |
| | | cytoplasmic tail |
| 26 | MKGKNPSKVIKSYYGTMDIKKINEGLLDSK | NivG_CT_30 |
| | | cytoplasmic tail |
| 27 | MNTTSDKGKNPSKVIKSYYGTMDIKKINEGLLDSK | NivG_CT_35 |
| | | cytoplasmic tail |
| 28 | MKVRFENTTSDKGKNPSKVIKSYYGTMDIKKINEGLLDSK | NivG_CT_40 |
| | | cytoplasmic tail |
| 29 | MIIKSYYGTMDIKKINEGLLDSK | NivG_CSUR38_CT_23 |
| | | cytoplasmic tail |
| 30 | MGKIPSKIIKSYYGTMDIKKINEGLLDSK | NivG_CSUR38_CT_29 |
| | | cytoplasmic tail |
| 31 | MNTTSDKGKIPSKIIKSYYGTMDIKKINEGLLDSK | NivG_CSUR38_CT_35 |
| | | cytoplasmic tail |
| 32 | MSKKVRFENTTSDKGKIPSKIIKSYYGTMDIKKINEGLLDSK | NivG_CSUR38_CT_42 |
| | | cytoplasmic tail |
| 33 | MPAESKKVRFENTTSDKGKIPSKIIKSYYGTMDIKKINEGLLDS | NivG_CSUR38_CT_45_ |
| | K | FL cytoplasmic tail |
| 34 | MNPSKVIKSYYGTMDIKKINEGLLDSK | NivG_VRI- |
| | | 0626_CT_27 |
| | | cytoplasmic tail |
| 35 | MDKGKNPSKVIKSYYGTMDIKKINEGLLDSK | NivG_VRI- |
| | | 0626_CT_31 |
| | | cytoplasmic tail |
| 36 | MNTTSDKGKNPSKVIKSYYGTMDIKKINEGLLDSK | NivG_VRI- |
| | | 0626_CT_35 |
| 37 | MKVRFENTTSDKGKNPSKVIKSYYGTMDIKKINEGLLDSK | NivG_VRI- |
| | | 0626_CT_40 |
| | | cytoplasmic tail |
| 38 | MPAENKKVRFENTTSDKGKNPSKVIKSYYGTMDIKKINEGLL | NivG_VRI- |
| | DSK | 0626_CT_45_FL |
| | | cytoplasmic tail |
| 39 | MKKINEGLLDSK | NivG_CT_12 |
| | | cytoplasmic tail |
| 40 | MDGLLDSK | HevG_CT_08 |
| 41 | MNDGLLDSK | HevG_CT_09 |
| 42 | MINDGLLDSK | HevG_CT_10 |
| 43 | MKINDGLLDSK | HevG_CT_11 |
| 44 | MKKINDGLLDSK | HevG_CT_12 |
| 45 | MIKKINDGLLDSK | HevG_CT_13 |
| 46 | MDIKKINDGLLDSK | HevG_CT_14 |
| 47 | MMDIKKINDGLLDSK | HevG_CT_15 |
| 48 | MTMDIKKINDGLLDSK | HevG_CT_16 |
| 49 | MGTMDIKKINDGLLDSK | HevG_CT_17 |
| 50 | MYGTMDIKKINDGLLDSK | HevG_CT_18 |
| 51 | MYYGTMDIKKINDGLLDSK | HevG_CT_19 |
| 52 | MNYYGTMDIKKINDGLLDSK | HevG_CT_20 |
| 53 | MKNYYGTMDIKKINDGLLDSK | HevG_CT_21 |
| 54 | MIKNYYGTMDIKKINDGLLDSK | HevG_CT_22 |
| 55 | MVIKNYYGTMDIKKINDGLLDSK | HevG_CT_23 |
| 56 | MKVIKNYYGTMDIKKINDGLLDSK | HevG_CT_24 |
| 57 | MMADSKLVSLNNNLSGKIKDQGKVIKNYYGTMDIKKINDGL | HevG_CT_45_FL |
| | LDSK | |
| 58 | MQNDHY | BatPV_G_CT_6 |
| 59 | MNQNDHY | BatPV_G_CT_7 |
| 60 | MKNQNDHY | BatPV_G_CT_8 |
| 61 | MQKNQNDHY | BatPV_G_CT_9 |
| 62 | MKQKNQNDHY | BatPV_G_CT_10 |
| 63 | MKKQKNQNDHY | BatPV_G_CT_11 |
| 64 | MWKKQKNQNDHY | BatPV_G_CT_12 |
| 65 | MNWKKQKNQNDHY | BatPV_G_CT_13 |
| 66 | MRNWKKQKNQNDHY | BatPV_G_CT_14 |
| 67 | MERNWKKQKNQNDHY | BatPV_G_CT_15 |
| 68 | MSERNWKKQKNQNDHY | BatPV_G_CT_16 |
| 69 | MHSERNWKKQKNQNDHY | BatPV_G_CT_17 |
| 70 | MSHSERNWKKQKNQNDHY | BatPV_G_CT_18 |
| 71 | MGSHSERNWKKQKNQNDHY | BatPV_G_CT_19 |
| 72 | MLGSHSERNWKKQKNQNDHY | BatPV_G_CT_20 |
| 73 | MGLGSHSERNWKKQKNQNDHY | BatPV_G_CT_21 |
| 74 | MFGLGSHSERNWKKQKNQNDHY | BatPV_G_CT_22 |
| 75 | MYFGLGSHSERNWKKQKNQNDHY | BatPV_G_CT_23 |
| 76 | MGYFGLGSHSERNWKKQKNQNDHY | BatPV_G_CT_24 |
| 77 | MKITKQGYFGLGSHSERNWKKQKNQNDHY | BatPV_G_CT_29 |
| 78 | MPQKTVEFINMNSPLERGVSTLSDKKTLNQSKITKQGYFGLGS | BatPV_G_CT_59_FL |
| | HSERNWKKQKNQNDHY | |
| 79 | MLHDIK | CedarV_G_CT_6 |
| 80 | MSLHDIK | CedarV_G_CT_7 |
| 81 | MESLHDIK | CedarV_G_CT_8 |
| 82 | MNESLHDIK | CedarV_G_CT_9 |
| 83 | MLNESLHDIK | CedarV_G_CT_10 |
| 84 | MLLNESLHDIK | CedarV_G_CT_11 |
| 85 | MNLLNESLHDIK | CedarV_G_CT_12 |
| 86 | MSNLLNESLHDIK | CedarV_G_CT_13 |
| 87 | MVSNLLNESLHDIK | CedarV_G_CT_14 |
| 88 | MNVSNLLNESLHDIK | CedarV_G_CT_15 |
| 89 | MYNVSNLLNESLHDIK | CedarV_G_CT_16 |
| 90 | MNYNVSNLLNESLHDIK | CedarV_G_CT_17 |
| 91 | MKNYNVSNLLNESLHDIK | CedarV_G_CT_18 |
| 92 | MNKNYNVSNLLNESLHDIK | CedarV_G_CT_19 |
| 93 | MKNKNYNVSNLLNESLHDIK | CedarV_G_CT_20 |
| 94 | MVKNKNYNVSNLLNESLHDIK | CedarV_G_CT_21 |
| 95 | MYVKNKNYNVSNLLNESLHDIK | CedarV_G_CT_22 |
| 96 | MYYVKNKNYNVSNLLNESLHDIK | CedarV_G_CT_23 |
| 97 | MSYYVKNKNYNVSNLLNESLHDIK | CedarV_G_CT_24 |
| 98 | MQKDLNKSYYVKNKNYNVSNLLNESLHDIK | CedarV_G_CT_30 |
| 99 | MLSQLQKNYLDNSNQQGDKMNNPDKKLSVNFNPLELDKGQ | CedarV_G_CT_68_FL |
| | KDLNKSYYVKNKNYNVSNLLNESLHDIK | |
| 100 | MQGGRR | CDV-H_CT_06 |
| 101 | MEQGGRR | CDV-H_CT_07 |
| 102 | MEEQGGRR | CDV-H_CT_08 |
| 103 | MTEEQGGRR | CDV-H_CT_09 |
| 104 | MVTEEQGGRR | CDV-H_CT_10 |
| 105 | MLVTEEQGGRR | CDV-H_CT_11 |
| 106 | MSLVTEEQGGRR | CDV-H_CT_12 |
| 107 | MLSLVTEEQGGRR | CDV-H_CT_13 |
| 108 | MKLSLVTEEQGGRR | CDV-H_CT_14 |
| 109 | MSKLSLVTEEQGGRR | CDV-H_CT_15 |
| 110 | MSSKLSLVTEEQGGRR | CDV-H_CT_16 |
| 111 | MNSSKLSLVTEEQGGRR | CDV-H_CT_17 |
| 112 | MANSSKLSLVTEEQGGRR | CDV-H_CT_18 |
| 113 | MRANSSKLSLVTEEQGGRR | CDV-H_CT_19 |
| 114 | MARANSSKLSLVTEEQGGRR | CDV-H_CT_20 |
| 115 | MLSYQDKVGAFYKDNARANSSKLSLVTEEQGGRR | CDV-H_CT_34_FL |
| 116 | MRIIFR | HPIV2-HN_CT_06 |
| 117 | MIPKRTCRIIFR | HPIV2-HN_CT_12 |
| 118 | MEDYSNLSLKSIPKRTCRIIFR | HPIV2-HN_CT_22_FL |
| 119 | MLMIDR | MvH_CT_06 |
| 120 | MHLMIDR | MvH_CT_07 |
| 121 | MEHLMIDR | MvH_CT_08 |
| 122 | MREHLMIDR | MvH_CT_09 |
| 123 | MNREHLMIDR | MvH_CT_10 |
| 124 | MINREHLMIDR | MvH_CT_11 |
| 125 | MVINREHLMIDR | MvH_CT_12 |
| 126 | MIVINREHLMIDR | MvH_CT_13 |
| 127 | MRIVINREHLMIDR | MvH_CT_14 |
| 128 | MSRIVINREHLMIDR | MvH_CT_15 |
| 129 | MGSRIVINREHLMIDR | MvH_CT_16 |
| 130 | MKGSRIVINREHLMIDR | MvH_CT_17 |
| 131 | MPKGSRIVINREHLMIDR | MvH_CT_18 |
| 132 | MHPKGSRIVINREHLMIDR | MvH_CT_19 |
| 133 | MPHPKGSRIVINREHLMIDR | MvH_CT_20 |
| 134 | MSPQRDRINAFYKDNPHPKGSRIVINREHLMIDR | MvH_CT_34_FL |
| 135 | MRLVFR | NDV-HN_CT_06 |
| 136 | MWRLVFR | NDV-HN_CT_07 |
| 137 | MTWRLVFR | NDV-HN_CT_08 |
| 138 | MNTWRLVFR | NDV-HN_CT_09 |
| 139 | MKNTWRLVFR | NDV-HN_CT_10 |
| 140 | MAKNTWRLVFR | NDV-HN_CT_11 |
| 141 | MEAKNTWRLVFR | NDV-HN_CT_12 |
| 142 | MREAKNTWRLVFR | NDV-HN_CT_13 |
| 143 | MEREAKNTWRLVFR | NDV-HN_CT_14 |
| 144 | MDEREAKNTWRLVFR | NDV-HN_CT_15 |
| 145 | MNDEREAKNTWRLVFR | NDV-HN_CT_16 |
| 146 | MENDEREAKNTWRLVFR | NDV-HN_CT_17 |
| 147 | MLENDEREAKNTWRLVFR | NDV-HN_CT_18 |
| 148 | MALENDEREAKNTWRLVFR | NDV-HN_CT_19 |
| 149 | MVALENDEREAKNTWRLVFR | NDV-HN_CT_20 |
| 150 | MERGVSQVALENDEREAKNTWRLVFR | NDV-HN_CT_26_FL |
| 151 | MERSGK | SeV_CT_06 |
| 152 | MSERSGK | SeV_CT_07 |
| 153 | MDSERSGK | SeV_CT_08 |
| 154 | MSDSERSGK | SeV_CT_09 |
| 155 | MVSDSERSGK | SeV_CT_10 |
| 156 | MLVSDSERSGK | SeV_CT_11 |
| 157 | MKLVSDSERSGK | SeV_CT_12 |
| 158 | MTKLVSDSERSGK | SeV_CT_13 |
| 159 | MTTKLVSDSERSGK | SeV_CT_14 |
| 160 | MSTTKLVSDSERSGK | SeV_CT_15 |
| 161 | MGSTTKLVSDSERSGK | SeV_CT_16 |
| 162 | MGGSTTKLVSDSERSGK | SeV_CT_17 |
| 163 | MPGGSTTKLVSDSERSGK | SeV_CT_18 |
| 164 | MSPGGSTTKLVSDSERSGK | SeV_CT_19 |
| 165 | MTSPGGSTTKLVSDSERSGK | SeV_CT_20 |
| 166 | MDGDRSKRDSYWSTSPGGSTTKLVSDSERSGK | SeV_CT_32_FL |
| 167 | MDQAEEDTRLVQYQQTLVMAHIINLKDNIFATLRN | BaEVwt_CT |
| 168 | MAHIINLKDNIFATLRNKKINEGLLDSK | BaEVRLess_CT |
| 169 | MAHIINLKDNIFATLRN | BaEVRLess_CT |
| 170 | MKRFRSMEIDNYIRKNNSGQYRYR | Cocal_CT |
| 171 | MKRFRSMEIDNYIRKNNSGQYRYRKKINEGLLDSK | Cocal_CT |
| 172 | MFVFKC | EboV-GP_CT |
| 173 | MFVFKCKKINEGLLDSK | EboV-GP_CT |
| 174 | MLNGENELAQYKQRLVLIKVASIRDNIFQVLKKKINEGLLDSK | GaLV_CT |
| 175 | MLNGENELAQYKQRLVLIKVASIRDNIFQVLK | GaLV_CT |
| 176 | MLIKVASIRDNIFQVLKKKINEGLLDSK | GaLV-RLess_CT |
| 177 | MLIKVASIRDNIFQVLK | GaLV-RLess_CT |
| 178 | MRRKWVTKVGPVKFAGCSCIGKNTLRHPKPCSGGKIHRHT | LCMV_GP_CT |
| 179 | MPEYEIPKLQHYQQTLVLAQVVSIRDKVFQVLRKKINEGLLDS | MLV-A_CT |
| | K | |
| 180 | MPEYEIPKLQHYQQTLVLAQVVSIRDKVFQVLR | MLV-A_CT |
| 181 | MLAQVVSIRDKVFQVLRKKINEGLLDSK | MLV-A-RLess_CT |
| 182 | MLAQVVSIRDKVFQVLR | MLV-A-RLess_CT |
| 183 | MKGLRNMEIDTYIQRKKTHKLKICLHIGVR | VSV-G_CT |
| 184 | MKGLRNMEIDTYIQRKKTHKLKICLHIGVRKKINEGLLDSK | VSV-G_CT |
| 185 | MLLIRELGQRIRRPIHRIARYAAQLVEIVRDTGEAVAIATANLL | HIV-1_gp41_MLLIR- |
| | NVASNKLEQSWYQLLNWWYKLAEWGRRGLLEVIRTVILLLD | LLP1-LLP3-LLP2- |
| | RLRHYSFLCLSRLDDWILALSGNVLRISRDRDREGGEEEIGEPR | gp41-CT-N |
| | DPGRPIPLHTQFSLPSYGQRVRN | |
| 186 | MLLIRELGQRIRRPIHRIARYAAQLVEIVRDTGEAVAIATANLL | HIV-1_gp41_MLLIR- |
| | NVASNKLEQSWYQLLNWWYKLAEWGRRGLLEVIRTVILLLD | LLP1-LLP3-LLP2- |
| | RLRHYSFLCLSRLDDWILALSGNVLRISRDRDREGGEEEIGEPR | gp41-CT-N |
| | DPGRPIPLHTQFSLPSYGQRVRNKKINEGLLDSK | |
| 187 | MAVAIATANLLNVASNKLEQSWYQLLNWWYKLAEWGRRGL | HIV-1_gp41_LLP3- |
| | LEVIRTVILLLDRLRHYSFLCLSRLDDWILALSGNVLRISRDRD | LLP2-gp41-CT-N |
| | REGGEEEIGEPRDPGRPIPLHTQFSLPSYGQRVRN | |
| 188 | MLLEVIRTVILLLDRLRHYSFLCLSRLDDWILALSGNVLRISRD | HIV-1_gp41_LLP2- |
| | RDREGGEEEIGEPRDPGRPIPLHTQFSLPSYGQRVRN | gp41-CT-N |
| 189 | MSGNVLRISRDRDREGGEEEIGEPRDPGRPIPLHTQFSLPSYGQ | HIV-1_gp41_gp41-CT- |
| | RVRN | N |
| 190 | MLLIRELGQRIRRPIHRIARYAAQLVEIVRDTGEAVAIATANLL | HIV-1_gp41_MLLIR- |
| | NVASNKLEQSWYQLLNWWYKLAEWGRRGLLEVIRTVILLLD | LLP1-LLP3-LLP2 |
| | RLRHYSFLCLSRLDDWILAL | |
| 191 | MELGQRIRRPIHRIARYAAQLVEIVRDTGEAVAIATANLLNVA | HIV-1_gp41_LLP1- |
| | SNKLEQSWYQLLNWWYKLAEWGRRGLLEVIRTVILLLDRLR | LLP3-LLP2 |
| | HYSFLCLSRLDDWILAL | |
| 192 | MAVAIATANLLNVASNKLEQSWYQLLNWWYKLAEWGRRGL | HIV-1_gp41_LLP3- |
| | LEVIRTVILLLDRLRHYSFLCLSRLDDWILAL | LLP2 |
| 193 | MLLEVIRTVILLLDRLRHYSFLCLSRLDDWILAL | HIV-1_gp41_LLP2 |
| 194 | MLLIRELGQRIRRPIHRIARYAAQLVEIVRDTGEAVAIATANLL | HIV-1_gp41_MLLIR- |
| | NVASNKLEQSWYQLLNWWYKLAEW | LLP1-LLP3 |
| 195 | MELGQRIRRPIHRIARYAAQLVEIVRDTGEAVAIATANLLNVA | HIV-1_gp41_LLP1- |
| | SNKLEQSWYQLLNWWYKLAEW | LLP3 |
| 196 | MAVAIATANLLNVASNKLEQSWYQLLNWWYKLAEW | HIV-1_gp41_LLP3 |
| 197 | MLLIRELGQRIRRPIHRIARYAAQLVEIVR | HIV-1_gp41_MLLIR- |
| | | LLP1 |
| 198 | MELGQRIRRPIHRIARYAAQLVEIVR | HIV-1_gp41_LLP1 |
| 199 | MLLIR | HIV-1_gp41_MLLIR |
| 200 | MAVEGGMKCVK | CD63_CT_1to11 |
| 201 | MCGACKENYC | CD63_CT_73to81 |
| 202 | MVEYGSRISKVLCC | CD63_CT_225to238 |
| 203 | MAVEGGMKCVKKKINEGLLDSK | CD63_CT_1to11 |
| 204 | MCGACKENYCKKINEGLLDSK | CD63_CT_73to81 |
| 205 | MVEYGSRISKVLCCKKINEGLLDSK | CD63_CT_225to238 |
| 206 | MKGEYKPNVVTTVASKYIPNEGTDWKANMKEKEFKAFERRD | CD29_CT |
| | HIIMLLK | |
| 207 | MKGEYKPNVVTTVASKYIPNEGTDWKANMKEKEFKAFERRD | CD29_CT |
| | HIIMLLKKKINEGLLDSK | |
| 208 | MVINREHLMIDRILSAFNTVIALLGSIVIIVMNIMIIQNYTRSTD | MvH_CT_12 |
| | NQAVIKDALQGIQQQIKGLADKIGTEIGPKVSLIDTSSTITIPANI | |
| | GLLGSKISQSTASINENVNEKCKFTLPPLKIHECNISCPNPLPFR | |
| | EYRPQTEGVSNLVGLPNNICLQKTSNQILKPKLISYTLPVVGQS | |
| | GTCITDPLLAMDEGYFAYSHLERIGSCSRGVSKQRIIGVGEVL | |
| | DRGDEVPSLFMTNVWTPPNPNTVYHCSAVYNNEFYYVLCAV | |
| | STVGDPILNSTYWSGSLMMTRLAVKPKSNGGGYNQHQLALR | |
| | SIEKGRYDKVMPYGPSGIKQGDTLYFPAVGFLVRTEFKYNDS | |
| | NCPITKCQYSKPENCRLSMGIRPNSHYILRSGLLKYNLSDGENP | |
| | KVVFIEISDQRLSIGSPSKIYDSLGQPVFYQASFSWDTMIKFGD | |
| | VLTVNPLVVNWRNNTVISRPGQSQCPRFNTCPAICAEGVYND | |
| | AFLIDRINWISAGVFLDSNATAANPVFTVFKDNEILYRAQLAS | |
| | EDTNAQKTITNCFLLKNKIWCISLVEIYDTGDNVIRPKLFAVKI | |
| | PEQCT | |
| 209 | MGSRIVINREHLMIDRILSAFNTVIALLGSIVIIVMNIMIIQNYTR | MvH_CT_16 |
| | STDNQAVIKDALQGIQQQIKGLADKIGTEIGPKVSLIDTSSTITI | |
| | PANIGLLGSKISQSTASINENVNEKCKFTLPPLKIHECNISCPNP | |
| | LPFREYRPQTEGVSNLVGLPNNICLQKTSNQILKPKLISYTLPV | |
| | VGQSGTCITDPLLAMDEGYFAYSHLERIGSCSRGVSKQRIIGV | |
| | GEVLDRGDEVPSLFMTNVWTPPNPNTVYHCSAVYNNEFYYV | |
| | LCAVSTVGDPILNSTYWSGSLMMTRLAVKPKSNGGGYNQHQ | |
| | LALRSIEKGRYDKVMPYGPSGIKQGDTLYFPAVGFLVRTEFKY | |
| | NDSNCPITKCQYSKPENCRLSMGIRPNSHYILRSGLLKYNLSD | |
| | GENPKVVFIEISDQRLSIGSPSKIYDSLGQPVFYQASFSWDTMI | |
| | KFGDVLTVNPLVVNWRNNTVISRPGQSQCPRENTCPAICAEG | |
| | VYNDAFLIDRINWISAGVFLDSNATAANPVFTVFKDNEILYRA | |
| | QLASEDTNAQKTITNCFLLKNKIWCISLVEIYDTGDNVIRPKLF | |
| | AVKIPEQCT | |
| 210 | MPHPKGSRIVINREHLMIDRILSAFNTVIALLGSIVIIVMNIMIIQ | MvH_CT_20 |
| | NYTRSTDNQAVIKDALQGIQQQIKGLADKIGTEIGPKVSLIDTS | |
| | STITIPANIGLLGSKISQSTASINENVNEKCKFTLPPLKIHECNIS | |
| | CPNPLPFREYRPQTEGVSNLVGLPNNICLQKTSNQILKPKLISY | |
| | TLPVVGQSGTCITDPLLAMDEGYFAYSHLERIGSCSRGVSKQR | |
| | IIGVGEVLDRGDEVPSLFMTNVWTPPNPNTVYHCSAVYNNEF | |
| | YYVLCAVSTVGDPILNSTYWSGSLMMTRLAVKPKSNGGGYN | |
| | QHQLALRSIEKGRYDKVMPYGPSGIKQGDTLYFPAVGFLVRT | |
| | EFKYNDSNCPITKCQYSKPENCRLSMGIRPNSHYILRSGLLKY | |
| | NLSDGENPKVVFIEISDQRLSIGSPSKIYDSLGQPVFYQASFSW | |
| | DTMIKFGDVLTVNPLVVNWRNNTVISRPGQSQCPRFNTCPAIC | |
| | AEGVYNDAFLIDRINWISAGVFLDSNATAANPVFTVFKDNEIL | |
| | YRAQLASEDTNAQKTITNCFLLKNKIWCISLVEIYDTGDNVIRP | |
| | KLFAVKIPEQCT | |
| 211 | MYYGTMDIKKINEGLLDSKILSAFNTVIALLGSIVIIVMNIMIIQ | NivG_CT_19 |
| | NYTRSTDNQAVIKDALQGIQQQIKGLADKIGTEIGPKVSLIDTS | |
| | STITIPANIGLLGSKISQSTASINENVNEKCKFTLPPLKIHECNIS | |
| | CPNPLPFREYRPQTEGVSNLVGLPNNICLQKTSNQILKPKLISY | |
| | TLPVVGQSGTCITDPLLAMDEGYFAYSHLERIGSCSRGVSKQR | |
| | IIGVGEVLDRGDEVPSLFMTNVWTPPNPNTVYHCSAVYNNEF | |
| | YYVLCAVSTVGDPILNSTYWSGSLMMTRLAVKPKSNGGGYN | |
| | QHQLALRSIEKGRYDKVMPYGPSGIKQGDTLYFPAVGFLVRT | |
| | EFKYNDSNCPITKCQYSKPENCRLSMGIRPNSHYILRSGLLKY | |
| | NLSDGENPKVVFIEISDQRLSIGSPSKIYDSLGQPVFYQASFSW | |
| | DTMIKFGDVLTVNPLVVNWRNNTVISRPGQSQCPRFNTCPAIC | |
| | AEGVYNDAFLIDRINWISAGVFLDSNATAANPVFTVFKDNEIL | |
| | YRAQLASEDTNAQKTITNCFLLKNKIWCISLVEIYDTGDNVIRP | |
| | KLFAVKIPEQCT | |
| 212 | MKNQNDHYILSAFNTVIALLGSIVIIVMNIMIIQNYTRSTDNQA | BatPV_G_CT_8 |
| | VIKDALQGIQQQIKGLADKIGTEIGPKVSLIDTSSTITIPANIGLL | |
| | GSKISQSTASINENVNEKCKFTLPPLKIHECNISCPNPLPFREYR | |
| | PQTEGVSNLVGLPNNICLQKTSNQILKPKLISYTLPVVGQSGTC | |
| | ITDPLLAMDEGYFAYSHLERIGSCSRGVSKQRIIGVGEVLDRG | |
| | DEVPSLFMTNVWTPPNPNTVYHCSAVYNNEFYYVLCAVSTV | |
| | GDPILNSTYWSGSLMMTRLAVKPKSNGGGYNQHQLALRSIEK | |
| | GRYDKVMPYGPSGIKQGDTLYFPAVGFLVRTEFKYNDSNCPI | |
| | TKCQYSKPENCRLSMGIRPNSHYILRSGLLKYNLSDGENPKVV | |
| | FIEISDQRLSIGSPSKIYDSLGQPVFYQASFSWDTMIKFGDVLTV | |
| | NPLVVNWRNNTVISRPGQSQCPRFNTCPAICAEGVYNDAFLID | |
| | RINWISAGVFLDSNATAANPVFTVFKDNEILYRAQLASEDTNA | |
| | QKTITNCFLLKNKIWCISLVEIYDTGDNVIRPKLFAVKIPEQCT | |
| 213 | MDSERSGKILSAFNTVIALLGSIVIIVMNIMIIQNYTRSTDNQAV | SeV_CT_08 |
| | IKDALQGIQQQIKGLADKIGTEIGPKVSLIDTSSTITIPANIGLLG | |
| | SKISQSTASINENVNEKCKFTLPPLKIHECNISCPNPLPFREYRP | |
| | QTEGVSNLVGLPNNICLQKTSNQILKPKLISYTLPVVGQSGTCI | |
| | TDPLLAMDEGYFAYSHLERIGSCSRGVSKQRIIGVGEVLDRGD | |
| | EVPSLFMTNVWTPPNPNTVYHCSAVYNNEFYYVLCAVSTVG | |
| | DPILNSTYWSGSLMMTRLAVKPKSNGGGYNQHQLALRSIEKG | |
| | RYDKVMPYGPSGIKQGDTLYFPAVGFLVRTEFKYNDSNCPIT | |
| | KCQYSKPENCRLSMGIRPNSHYILRSGLLKYNLSDGENPKVVF | |
| | IEISDQRLSIGSPSKIYDSLGQPVFYQASFSWDTMIKFGDVLTV | |
| | NPLVVNWRNNTVISRPGQSQCPRFNTCPAICAEGVYNDAFLID | |
| | RINWISAGVFLDSNATAANPVFTVFKDNEILYRAQLASEDTNA | |
| | QKTITNCFLLKNKIWCISLVEIYDTGDNVIRPKLFAVKIPEQCT | |
| 214 | MLAQVVSIRDKVFQVLRILSAFNTVIALLGSIVIIVMNIMIIQNY | MLV-A-RLess_CT |
| | TRSTDNQAVIKDALQGIQQQIKGLADKIGTEIGPKVSLIDTSSTI | |
| | TIPANIGLLGSKISQSTASINENVNEKCKFTLPPLKIHECNISCPN | |
| | PLPFREYRPQTEGVSNLVGLPNNICLQKTSNQILKPKLISYTLP | |
| | VVGQSGTCITDPLLAMDEGYFAYSHLERIGSCSRGVSKQRIIG | |
| | VGEVLDRGDEVPSLFMTNVWTPPNPNTVYHCSAVYNNEFYY | |
| | VLCAVSTVGDPILNSTYWSGSLMMTRLAVKPKSNGGGYNQH | |
| | QLALRSIEKGRYDKVMPYGPSGIKQGDTLYFPAVGFLVRTEFK | |
| | YNDSNCPITKCQYSKPENCRLSMGIRPNSHYILRSGLLKYNLS | |
| | DGENPKVVFIEISDQRLSIGSPSKIYDSLGQPVFYQASFSWDTM | |
| | IKFGDVLTVNPLVVNWRNNTVISRPGQSQCPRFNTCPAICAEG | |
| | VYNDAFLIDRINWISAGVFLDSNATAANPVFTVFKDNEILYRA | |
| | QLASEDTNAQKTITNCFLLKNKIWCISLVEIYDTGDNVIRPKLF | |
| | AVKIPEQCT | |
| 215 | MLLIRILSAFNTVIALLGSIVIIVMNIMIIQNYTRSTDNQAVIKD | HIV-1_gp41_MLLIR |
| | ALQGIQQQIKGLADKIGTEIGPKVSLIDTSSTITIPANIGLLGSKI | |
| | SQSTASINENVNEKCKFTLPPLKIHECNISCPNPLPFREYRPQTE | |
| | GVSNLVGLPNNICLQKTSNQILKPKLISYTLPVVGQSGTCITDP | |
| | LLAMDEGYFAYSHLERIGSCSRGVSKQRIIGVGEVLDRGDEVP | |
| | SLFMTNVWTPPNPNTVYHCSAVYNNEFYYVLCAVSTVGDPIL | |
| | NSTYWSGSLMMTRLAVKPKSNGGGYNQHQLALRSIEKGRYD | |
| | KVMPYGPSGIKQGDTLYFPAVGFLVRTEFKYNDSNCPITKCQ | |
| | YSKPENCRLSMGIRPNSHYILRSGLLKYNLSDGENPKVVFIEIS | |
| | DQRLSIGSPSKIYDSLGQPVFYQASFSWDTMIKFGDVLTVNPL | |
| | VVNWRNNTVISRPGQSQCPRFNTCPAICAEGVYNDAFLIDRIN | |
| | WISAGVFLDSNATAANPVFTVFKDNEILYRAQLASEDTNAQK | |
| | TITNCFLLKNKIWCISLVEIYDTGDNVIRPKLFAVKIPEQCT | |
| 216 | MAVEGGMKCVKILSAFNTVIALLGSIVIIVMNIMIIQNYTRSTD | CD63_CT_1to11 |
| | NQAVIKDALQGIQQQIKGLADKIGTEIGPKVSLIDTSSTITIPANI | |
| | GLLGSKISQSTASINENVNEKCKFTLPPLKIHECNISCPNPLPFR | |
| | EYRPQTEGVSNLVGLPNNICLQKTSNQILKPKLISYTLPVVGQS | |
| | GTCITDPLLAMDEGYFAYSHLERIGSCSRGVSKQRIIGVGEVL | |
| | DRGDEVPSLFMTNVWTPPNPNTVYHCSAVYNNEFYYVLCAV | |
| | STVGDPILNSTYWSGSLMMTRLAVKPKSNGGGYNQHQLALR | |
| | SIEKGRYDKVMPYGPSGIKQGDTLYFPAVGFLVRTEFKYNDS | |
| | NCPITKCQYSKPENCRLSMGIRPNSHYILRSGLLKYNLSDGENP | |
| | KVVFIEISDQRLSIGSPSKIYDSLGQPVFYQASFSWDTMIKFGD | |
| | VLTVNPLVVNWRNNTVISRPGQSQCPRFNTCPAICAEGVYND | |
| | AFLIDRINWISAGVFLDSNATAANPVFTVFKDNEILYRAQLAS | |
| | EDTNAQKTITNCFLLKNKIWCISLVEIYDTGDNVIRPKLFAVKI | |
| | PEQCT | |
| 217 | MCGACKENYCILSAFNTVIALLGSIVIIVMNIMIIQNYTRSTDN | CD63_CT_73to81 |
| | QAVIKDALQGIQQQIKGLADKIGTEIGPKVSLIDTSSTITIPANIG | |
| | LLGSKISQSTASINENVNEKCKFTLPPLKIHECNISCPNPLPFRE | |
| | YRPQTEGVSNLVGLPNNICLQKTSNQILKPKLISYTLPVVGQSG | |
| | TCITDPLLAMDEGYFAYSHLERIGSCSRGVSKQRIIGVGEVLDR | |
| | GDEVPSLFMTNVWTPPNPNTVYHCSAVYNNEFYYVLCAVST | |
| | VGDPILNSTYWSGSLMMTRLAVKPKSNGGGYNQHQLALRSIE | |
| | KGRYDKVMPYGPSGIKQGDTLYFPAVGFLVRTEFKYNDSNCP | |
| | ITKCQYSKPENCRLSMGIRPNSHYILRSGLLKYNLSDGENPKV | |
| | VFIEISDQRLSIGSPSKIYDSLGQPVFYQASFSWDTMIKFGDVLT | |
| | VNPLVVNWRNNTVISRPGQSQCPRFNTCPAICAEGVYNDAFLI | |
| | DRINWISAGVFLDSNATAANPVFTVFKDNEILYRAQLASEDTN | |
| | AQKTITNCFLLKNKIWCISLVEIYDTGDNVIRPKLFAVKIPEQC | |
| | T | |
| 218 | MKKINDGLLDSKILSAFNTVIALLGSIVIIVMNIMIIQNYTRSTD | HevG_CT_12 |
| | NQAVIKDALQGIQQQIKGLADKIGTEIGPKVSLIDTSSTITIPANI | |
| | GLLGSKISQSTASINENVNEKCKFTLPPLKIHECNISCPNPLPFR | |
| | EYRPQTEGVSNLVGLPNNICLQKTSNQILKPKLISYTLPVVGQS | |
| | GTCITDPLLAMDEGYFAYSHLERIGSCSRGVSKQRIIGVGEVL | |
| | DRGDEVPSLFMTNVWTPPNPNTVYHCSAVYNNEFYYVLCAV | |
| | STVGDPILNSTYWSGSLMMTRLAVKPKSNGGGYNQHQLALR | |
| | SIEKGRYDKVMPYGPSGIKQGDTLYFPAVGFLVRTEFKYNDS | |
| | NCPITKCQYSKPENCRLSMGIRPNSHYILRSGLLKYNLSDGENP | |
| | KVVFIEISDQRLSIGSPSKIYDSLGQPVFYQASFSWDTMIKFGD | |
| | VLTVNPLVVNWRNNTVISRPGQSQCPRFNTCPAICAEGVYND | |
| | AFLIDRINWISAGVFLDSNATAANPVFTVFKDNEILYRAQLAS | |
| | EDTNAQKTITNCFLLKNKIWCISLVEIYDTGDNVIRPKLFAVKI | |
| | PEQCT | |
| 219 | MPEYEIPKLQHYQQTLVLAQVVSIRDKVFQVLRILSAFNTVIA | MLV-A_CT |
| | LLGSIVIIVMNIMIIQNYTRSTDNQAVIKDALQGIQQQIKGLAD | |
| | KIGTEIGPKVSLIDTSSTITIPANIGLLGSKISQSTASINENVNEKC | |
| | KFTLPPLKIHECNISCPNPLPFREYRPQTEGVSNLVGLPNNICLQ | |
| | KTSNQILKPKLISYTLPVVGQSGTCITDPLLAMDEGYFAYSHLE | |
| | RIGSCSRGVSKQRIIGVGEVLDRGDEVPSLFMTNVWTPPNPNT | |
| | VYHCSAVYNNEFYYVLCAVSTVGDPILNSTYWSGSLMMTRL | |
| | AVKPKSNGGGYNQHQLALRSIEKGRYDKVMPYGPSGIKQGD | |
| | TLYFPAVGFLVRTEFKYNDSNCPITKCQYSKPENCRLSMGIRP | |
| | NSHYILRSGLLKYNLSDGENPKVVFIEISDQRLSIGSPSKIYDSL | |
| | GQPVFYQASFSWDTMIKFGDVLTVNPLVVNWRNNTVISRPGQ | |
| | SQCPRFNTCPAICAEGVYNDAFLIDRINWISAGVFLDSNATAA | |
| | NPVFTVFKDNEILYRAQLASEDTNAQKTITNCFLLKNKIWCISL | |
| | VEIYDTGDNVIRPKLFAVKIPEQCT | |
| 220 | MLLDSKILSAFNTVIALLGSIVIIVMNIMIIQNYTRSTDNQAVIK | NivG_CT_06 |
| | DALQGIQQQIKGLADKIGTEIGPKVSLIDTSSTITIPANIGLLGSK | |
| | ISQSTASINENVNEKCKFTLPPLKIHECNISCPNPLPFREYRPQT | |
| | EGVSNLVGLPNNICLQKTSNQILKPKLISYTLPVVGQSGTCITD | |
| | PLLAMDEGYFAYSHLERIGSCSRGVSKQRIIGVGEVLDRGDEV | |
| | PSLFMTNVWTPPNPNTVYHCSAVYNNEFYYVLCAVSTVGDPI | |
| | LNSTYWSGSLMMTRLAVKPKSNGGGYNQHQLALRSIEKGRY | |
| | DKVMPYGPSGIKQGDTLYFPAVGFLVRTEFKYNDSNCPITKC | |
| | QYSKPENCRLSMGIRPNSHYILRSGLLKYNLSDGENPKVVFIEI | |
| | SDQRLSIGSPSKIYDSLGQPVFYQASFSWDTMIKFGDVLTVNP | |
| | LVVNWRNNTVISRPGQSQCPRFNTCPAICAEGVYNDAFLIDRI | |
| | NWISAGVFLDSNATAANPVFTVFKDNEILYRAQLASEDTNAQ | |
| | KTITNCFLLKNKIWCISLVEIYDTGDNVIRPKLFAVKIPEQCT | |
| 221 | MIKKINEGLLDSKILSAFNTVIALLGSIVIIVMNIMIIQNYTRSTD | NivG_CT_13 |
| | NQAVIKDALQGIQQQIKGLADKIGTEIGPKVSLIDTSSTITIPANI | |
| | GLLGSKISQSTASINENVNEKCKFTLPPLKIHECNISCPNPLPFR | |
| | EYRPQTEGVSNLVGLPNNICLQKTSNQILKPKLISYTLPVVGQS | |
| | GTCITDPLLAMDEGYFAYSHLERIGSCSRGVSKQRIIGVGEVL | |
| | DRGDEVPSLFMTNVWTPPNPNTVYHCSAVYNNEFYYVLCAV | |
| | STVGDPILNSTYWSGSLMMTRLAVKPKSNGGGYNQHQLALR | |
| | SIEKGRYDKVMPYGPSGIKQGDTLYFPAVGFLVRTEFKYNDS | |
| | NCPITKCQYSKPENCRLSMGIRPNSHYILRSGLLKYNLSDGENP | |
| | KVVFIEISDQRLSIGSPSKIYDSLGQPVFYQASFSWDTMIKFGD | |
| | VLTVNPLVVNWRNNTVISRPGQSQCPRFNTCPAICAEGVYND | |
| | AFLIDRINWISAGVFLDSNATAANPVFTVFKDNEILYRAQLAS | |
| | EDTNAQKTITNCFLLKNKIWCISLVEIYDTGDNVIRPKLFAVKI | |
| | PEQCT | |
| 222 | MWKKQKNQNDHYILSAFNTVIALLGSIVIIVMNIMIIQNYTRST | BatPV_G_CT_12 |
| | DNQAVIKDALQGIQQQIKGLADKIGTEIGPKVSLIDTSSTITIPA | |
| | NIGLLGSKISQSTASINENVNEKCKFTLPPLKIHECNISCPNPLPF | |
| | REYRPQTEGVSNLVGLPNNICLQKTSNQILKPKLISYTLPVVGQ | |
| | SGTCITDPLLAMDEGYFAYSHLERIGSCSRGVSKQRIIGVGEVL | |
| | DRGDEVPSLFMTNVWTPPNPNTVYHCSAVYNNEFYYVLCAV | |
| | STVGDPILNSTYWSGSLMMTRLAVKPKSNGGGYNQHQLALR | |
| | SIEKGRYDKVMPYGPSGIKQGDTLYFPAVGFLVRTEFKYNDS | |
| | NCPITKCQYSKPENCRLSMGIRPNSHYILRSGLLKYNLSDGENP | |
| | KVVFIEISDQRLSIGSPSKIYDSLGQPVFYQASFSWDTMIKFGD | |
| | VLTVNPLVVNWRNNTVISRPGQSQCPRFNTCPAICAEGVYND | |
| | AFLIDRINWISAGVFLDSNATAANPVFTVFKDNEILYRAQLAS | |
| | EDTNAQKTITNCFLLKNKIWCISLVEIYDTGDNVIRPKLFAVKI | |
| | PEQCT | |
| 223 | MRIIFRILSAFNTVIALLGSIVIIVMNIMIIQNYTRSTDNQAVIKD | HPIV2-HN_CT_06 |
| | ALQGIQQQIKGLADKIGTEIGPKVSLIDTSSTITIPANIGLLGSKI | |
| | SQSTASINENVNEKCKFTLPPLKIHECNISCPNPLPFREYRPQTE | |
| | GVSNLVGLPNNICLQKTSNQILKPKLISYTLPVVGQSGTCITDP | |
| | LLAMDEGYFAYSHLERIGSCSRGVSKQRIIGVGEVLDRGDEVP | |
| | SLFMTNVWTPPNPNTVYHCSAVYNNEFYYVLCAVSTVGDPIL | |
| | NSTYWSGSLMMTRLAVKPKSNGGGYNQHQLALRSIEKGRYD | |
| | KVMPYGPSGIKQGDTLYFPAVGFLVRTEFKYNDSNCPITKCQ | |
| | YSKPENCRLSMGIRPNSHYILRSGLLKYNLSDGENPKVVFIEIS | |
| | DQRLSIGSPSKIYDSLGQPVFYQASFSWDTMIKFGDVLTVNPL | |
| | VVNWRNNTVISRPGQSQCPRFNTCPAICAEGVYNDAFLIDRIN | |
| | WISAGVFLDSNATAANPVFTVFKDNEILYRAQLASEDTNAQK | |
| | TITNCFLLKNKIWCISLVEIYDTGDNVIRPKLFAVKIPEQCT | |
| 224 | MIPKRTCRIIFRILSAFNTVIALLGSIVIIVMNIMIIQNYTRSTDN | HPIV2-HN_CT_12 |
| | QAVIKDALQGIQQQIKGLADKIGTEIGPKVSLIDTSSTITIPANIG | |
| | LLGSKISQSTASINENVNEKCKFTLPPLKIHECNISCPNPLPFRE | |
| | YRPQTEGVSNLVGLPNNICLQKTSNQILKPKLISYTLPVVGQSG | |
| | TCITDPLLAMDEGYFAYSHLERIGSCSRGVSKQRIIGVGEVLDR | |
| | GDEVPSLFMTNVWTPPNPNTVYHCSAVYNNEFYYVLCAVST | |
| | VGDPILNSTYWSGSLMMTRLAVKPKSNGGGYNQHQLALRSIE | |
| | KGRYDKVMPYGPSGIKQGDTLYFPAVGFLVRTEFKYNDSNCP | |
| | ITKCQYSKPENCRLSMGIRPNSHYILRSGLLKYNLSDGENPKV | |
| | VFIEISDQRLSIGSPSKIYDSLGQPVFYQASFSWDTMIKFGDVLT | |
| | VNPLVVNWRNNTVISRPGQSQCPRENTCPAICAEGVYNDAFLI | |
| | DRINWISAGVFLDSNATAANPVFTVFKDNEILYRAQLASEDTN | |
| | AQKTITNCFLLKNKIWCISLVEIYDTGDNVIRPKLFAVKIPEQC | |
| | T | |
| 225 | MKLVSDSERSGKILSAFNTVIALLGSIVIIVMNIMIIQNYTRSTD | CD63_CT_1to11 |
| | NQAVIKDALQGIQQQIKGLADKIGTEIGPKVSLIDTSSTITIPANI | |
| | GLLGSKISQSTASINENVNEKCKFTLPPLKIHECNISCPNPLPFR | |
| | EYRPQTEGVSNLVGLPNNICLQKTSNQILKPKLISYTLPVVGQS | |
| | GTCITDPLLAMDEGYFAYSHLERIGSCSRGVSKQRIIGVGEVL | |
| | DRGDEVPSLFMTNVWTPPNPNTVYHCSAVYNNEFYYVLCAV | |
| | STVGDPILNSTYWSGSLMMTRLAVKPKSNGGGYNQHQLALR | |
| | SIEKGRYDKVMPYGPSGIKQGDTLYFPAVGFLVRTEFKYNDS | |
| | NCPITKCQYSKPENCRLSMGIRPNSHYILRSGLLKYNLSDGENP | |
| | KVVFIEISDQRLSIGSPSKIYDSLGQPVFYQASFSWDTMIKFGD | |
| | VLTVNPLVVNWRNNTVISRPGQSQCPRFNTCPAICAEGVYND | |
| | AFLIDRINWISAGVFLDSNATAANPVFTVFKDNEILYRAQLAS | |
| | EDTNAQKTITNCFLLKNKIWCISLVEIYDTGDNVIRPKLFAVKI | |
| | PEQCT | |
| 226 | MVVILDKRCY CNLLILILMI SECSVGILHY EKLSKIGLVK | Truncated NiV fusion |
| | GVTRKYKIKS NPLTKDIVIK MIPNVSNMSQ CTGSVMENYK | glycoprotein |
| | TRLNGILTPI KGALEIYKNN THDLVGDVRL AGVIMAGVAI | (FcDelta22) at |
| | GIATAAQITA GVALYEAMKN ADNINKLKSS IESTNEAVVK | cytoplasmic tail |
| | LQETAEKTVY VLTALQDYIN TNLVPTIDKI SCKQTELSLD | |
| | LALSKYLSDL LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK KRNT | |
| 227 | ILHY EKLSKIGLVK GVTRKYKIKS NPLTKDIVIK | Truncated mature NiV |
| | MIPNVSNMSQ CTGSVMENYK TRLNGILTPI KGALEIYKNN | fusion glycoprotein |
| | THDLVGDVRL AGVIMAGVAI GIATAAQITA GVALYEAMKN | (FcDelta22) at |
| | ADNINKLKSS IESTNEAVVK LQETAEKTVY VLTALQDYIN | cytoplasmic tail |
| | TNLVPTIDKI SCKQTELSLD LALSKYLSDL LFVFGPNLQD | |
| | PVSNSMTIQA ISQAFGGNYE TLLRTLGYAT EDFDDLLESD | |
| | SITGQIIYVD LSSYYIIVRV YFPILTEIQQ AYIQELLPVS | |
| | FNNDNSEWIS IVPNFILVRN TLISNIEIGF CLITKRSVIC | |
| | NQDYATPMTN NMRECLTGST EKCPRELVVS SHVPRFALSN | |
| | GVLFANCISV TCQCQTTGRA ISQSGEQTLL MIDNTTCPTA | |
| | VLGNVIISLG KYLGSVNYNS EGIAIGPPVF TDKVDISSQI | |
| | SSMNQSLQQS KDYIKEAQRL LDTVNPSLIS MLSMIILYVL | |
| | SIASLCIGLI TFISFIIVEK KRNT | |
| 228 | MKKINEGLLDSKILSA FNTVIALLGS IVIIVMNIMI | NiVG protein |
| | IQNYTRSTDN QAVIKDALQG IQQQIKGLAD KIGTEIGPKV | attachment |
| | SLIDTSSTIT IPANIGLLGS KISQSTASIN ENVNEKCKFT | glycoprotein |
| | LPPLKIHECN ISCPNPLPFR EYRPQTEGVS NLVGLPNNIC | |
| | LQKTSNQILK PKLISYTLPV VGQSGTCITD PLLAMDEGYF | |
| | AYSHLERIGS CSRGVSKQRI IGVGEVLDRG DEVPSLFMTN | |
| | VWTPPNPNTV YHCSAVYNNE FYYVLCAVST VGDPILNSTY | |
| | WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRFNTC | |
| | PAICAEGVYN DAFLIDRINW ISAGVFLDSN ATAANPVFTV | |
| | FKDNEILYRA QLASEDTNAQ KTITNCFLLK NKIWCISLVE | |
| | IYDTGDNVIR PKLFAVKIPE QCT | |
| 229 | NRLVQFVKDRISVVQALVLTQQYHQLKPIEYEP | Moloney murine |
| | | leukemia virus |
| | | cytoplasmic tail (632- |
| | | 665 of UniProt P03385; |
| | | R-peptide 650-665) |
| 230 | GGGGS | peptide linker |
| 231 | GGGGGS | peptide linker |
| 232 | (GGGGS)n | peptide linker, wherein |
| | | n is 1 to 10 |
| 233 | (GGGGGS)n | peptide linker, wherein |
| | | n is 1 to 6 |
| 234 | MATQEVRLKCLLCGIIVLVLSLEGLGILHYEKLSKIGLVKGITR | Hendra F |
| | KYKIKSNPLTKDIVIKMIPNVSNVSKCTGTVMENYKSRLTGILS | |
| | PIKGAIELYNNNTHDLVGDVKLAGVVMAGIAIGIATAAQITAG | |
| | VALYEAMKNADNINKLKSSIESTNEAVVKLQETAEKTVYVLT | |
| | ALQDYINTNLVPTIDQISCKQTELALDLALSKYLSDLLFVFGPN | |
| | LQDPVSNSMTIQAISQAFGGNYETLLRTLGYATEDFDDLLESD | |
| | SIAGQIVYVDLSSYYIIVRVYFPILTEIQQAYVQELLPVSFNNDN | |
| | SEWISIVPNFVLIRNTLISNIEVKYCLITKKSVICNQDYATPMTA | |
| | SVRECLTGSTDKCPRELVVSSHVPRFALSGGVLFANCISVTCQ | |
| | CQTTGRAISQSGEQTLLMIDNTTCTTVVLGNIIISLGKYLGSINY | |
| | NSESIAVGPPVYTDKVDISSQISSMNQSLQQSKDYIKEAQKILD | |
| | TVNPSLISMLSMIILYVLSIAALCIGLITFISFVIVEKKRGNYSRL | |
| | DDRQVRPVSNGDLYYIGT | |
| 235 | MVVILDKRCYCNLLILILMISECSVGILHYEKLSKIGLVKGVTR | Nipah F0 |
| | KYKIKSNPLTKDIVIKMIPNVSNMSQCTGSVMENYKTRLNGIL | |
| | TPIKGALEIYKNNTHDLVGDVRLAGVIMAGVAIGIATAAQITA | |
| | GVALYEAMKNADNINKLKSSIESTNEAVVKLQETAEKTVYVL | |
| | TALQDYINTNLVPTIDKISCKQTELSLDLALSKYLSDLLFVFGP | |
| | NLQDPVSNSMTIQAISQAFGGNYETLLRTLGYATEDFDDLLES | |
| | DSITGQIIYVDLSSYYIIVRVYFPILTEIQQAYIQELLPVSFNNDN | |
| | SEWISIVPNFILVRNTLISNIEIGFCLITKRSVICNQDYATPMTNN | |
| | MRECLTGSTEKCPRELVVSSHVPRFALSNGVLFANCISVTCQC | |
| | QTTGRAISQSGEQTLLMIDNTTCPTAVLGNVIISLGKYLGSVN | |
| | YNSEGIAIGPPVFTDKVDISSQISSMNQSLQQSKDYIKEAQRLL | |
| | DTVNPSLISMLSMIILYVLSIASLCIGLITFISFIIVEKKRNTYSRL | |
| | EDRRVRPTSSGDLYYIGT | |
| 236 | MSNKRTTVLIIISYTLFYLNNAAIVGFDFDKLNKIGVVQGRVL | CedV F |
| | NYKIKGDPMTKDLVLKFIPNIVNITECVREPLSRYNETVRRLLL | |
| | PIHNMLGLYLNNTNAKMTGLMIAGVIMGGIAIGIATAAQITAG | |
| | FALYEAKKNTENIQKLTDSIMKTQDSIDKLTDSVGTSILILNKL | |
| | QTYINNQLVPNLELLSCRQNKIEFDLMLTKYLVDLMTVIGPNI | |
| | NNPVNKDMTIQSLSLLFDGNYDIMMSELGYTPQDFLDLIESKS | |
| | ITGQIIYVDMENLYVVIRTYLPTLIEVPDAQIYEFNKITMSSNG | |
| | GEYLSTIPNFILIRGNYMSNIDVATCYMTKASVICNQDYSLPMS | |
| | QNLRSCYQGETEYCPVEAVIASHSPRFALTNGVIFANCINTICR | |
| | CQDNGKTITQNINQFVSMIDNSTCNDVMVDKFTIKVGKYMGR | |
| | KDINNINIQIGPQIIIDKVDLSNEINKMNQSLKDSIFYLREAKRIL | |
| | DSVNISLISPSVQLFLIIISVLSFIILLIIIVYLYCKSKHSYKYNKFI | |
| | DDPDYYNDYKRERINGKASKSNNIYYVGD | |
| 237 | MALNKNMFSSLFLGYLLVYATTVQSSIHYDSLSKVGVIKGLT | Mojiang F |
| | YNYKIKGSPSTKLMVVKLIPNIDSVKNCTQKQYDEYKNLVRK | |
| | ALEPVKMAIDTMLNNVKSGNNKYRFAGAIMAGVALGVATA | |
| | ATVTAGIALHRSNENAQAIANMKSAIQNTNEAVKQLQLANKQ | |
| | TLAVIDTIRGEINNNIIPVINQLSCDTIGLSVGIRLTQYYSEIITAF | |
| | GPALQNPVNTRITIQAISSVFNGNFDELLKIMGYTSGDLYEILH | |
| | SELIRGNIIDVDVDAGYIALEIEFPNLTLVPNAVVQELMPISYNI | |
| | DGDEWVTLVPRFVLTRTTLLSNIDTSRCTITDSSVICDNDYALP | |
| | MSHELIGCLQGDTSKCAREKVVSSYVPKFALSDGLVYANCLN | |
| | TICRCMDTDTPISQSLGATVSLLDNKRCSVYQVGDVLISVGSY | |
| | LGDGEYNADNVELGPPIVIDKIDIGNQLAGINQTLQEAEDYIEK | |
| | SEEFLKGVNPSIITLGSMVVLYIFMILIAIVSVIALVLSIKLTVKG | |
| | NVVRQQFTYTQHVPSMENINYVSH | |
| 238 | MKKKTDNPTISKRGHNHSRGIKSRALLRETDNYSNGLIVENLV | Bat PV F |
| | RNCHHPSKNNLNYTKTQKRDSTIPYRVEERKGHYPKIKHLIDK | |
| | SYKHIKRGKRRNGHNGNIITIILLLILILKTQMSEGAIHYETLSKI | |
| | GLIKGITREYKVKGTPSSKDIVIKLIPNVTGLNKCTNISMENYK | |
| | EQLDKILIPINNIIELYANSTKSAPGNARFAGVIIAGVALGVAA | |
| | AAQITAGIALHEARQNAERINLLKDSISATNNAVAELQEATGG | |
| | IVNVITGMQDYINTNLVPQIDKLQCSQIKTALDISLSQYYSEILT | |
| | VFGPNLQNPVTTSMSIQAISQSFGGNIDLLLNLLGYTANDLLD | |
| | LLESKSITGQITYINLEHYFMVIRVYYPIMTTISNAYVQELIKISF | |
| | NVDGSEWVSLVPSYILIRNSYLSNIDISECLITKNSVICRHDFAM | |
| | PMSYTLKECLTGDTEKCPREAVVTSYVPRFAISGGVIYANCLS | |
| | TTCQCYQTGKVIAQDGSQTLMMIDNQTCSIVRIEEILISTGKYL | |
| | GSQEYNTMHVSVGNPVFTDKLDITSQISNINQSIEQSKFYLDKS | |
| | KAILDKINLNLIGSVPISILFIIAILSLILSIITFVIVMIIVRRYNKYT | |
| | PLINSDPSSRRSTIQDVYIIPNPGEHSIRSAARSIDRDRD | |
| 239 | DIQMTQTTSSLSASLGDRVTISCRASQDISKYLNWYQQKPDGT | Anti-CD19 scFv |
| | VKLLIYHTSRLHSGVPSRFSGSGSGTDYSLTISNLEQEDIATYF | (FMC63) |
| | CQQGNTLPYTFGGGTKLEITGSTSGSGKPGSGEGSTKGEVKLQ | |
| | ESGPGLVAPSQSLSVTCTVSGVSLPDYGVSWIRQPPRKGLEWL | |
| | GVIWGSETTYYNSALKSRLTIIKDNSKSQVFLKMNSLQTDDTA | |
| | IYYCAKHYYYGGSYAMDYWGQGTSVTVSS | |
| 240 | DIQMTQTTSSLSASLGDRVTISCRASQDISKYLNWYQQKPDGT | Anti-CD19 scFv |
| | VKLLIYHTSRLHSGVPSRFSGSGSGTDYSLTISNLEQEDIATYF | (FMC63) |
| | CQQGNTLPYTFGGGTKLEITGGGGSGGGGSGGGGSEVKLQES | |
| | GPGLVAPSQSLSVTCTVSGVSLPDYGVSWIRQPPRKGLEWLG | |
| | VIWGSETTYYNSALKSRLTIIKDNSKSQVFLKMNSLQTDDTAI | |
| | YYCAKHYYYGGSYAMDYWGQGTSVTVSS | |
| 241 | ESKYGPPCPPCP | IgG4 Hinge |
| 242 | TTTPAPRPPTPAPTIASQPLSLRPE | CD8 Hinge |
| 243 | IEVMYPPPYLDNEKSNGTIIHVKGKHLCPSPLFPGPSKP | CD28 |
| 244 | ACRPAAGGAVHTRGLDFACDIYIWAPLAGTCGVLLLSLVITLY | CD8 |
| | C | |
| 245 | FWVLVVVGGVLACYSLLVTVAFIIFWV | CD28 |
| 246 | FWVLVVVGGVLACYSLLVTVAFIIFWV | CD28 |
| 247 | RSKRSRLLHSDYMNMTPRRPGPTRKHYQPYAPPRDFAAYRS | CD28 |
| 248 | KRGRKKLLYIFKQPFMRPVQTTQEEDGCCRFPEEEEGGCEL | 4-1BB |
| 249 | RVKFSRSADAPAYQQGQNQLYNELNLGRREEYDVLDKRRGR | CD3zeta |
| | DPEMGGKPRRKNPQEGLYNELQKDKMAEAYSEIGMKGERRR | |
| | GKGHDGLYQGLSTATKDTYDALHMQALPPR | |
| 250 | RVKFSRSADAPAYKQGQNQLYNELNLGRREEYDVLDKRRGR | CD3zeta |
| | DPEMGGKPRRKNPQEGLYNELQKDKMAEAYSEIGMKGERRR | |
| | GKGHDGLYQGLSTATKDTYDALHMQALPPR | |
| 251 | AVGIGALFLGFLGAAGSTMGAASMTLTVQARQLLSGIVQQQN | P04578 (512-856) |
| | NLLRAIEAQQHLLQLTVWGIKQLQARILAVERYLKDQQLLGI | |
| | WGCSGKLICTTAVPWNASWSNKSLEQIWNHTTWMEW | |
| 252 | DREINNYTSLIHSLIEESQNQQEKNEQELLELDKWASLWNWF | HIV-1 Env (GP41) |
| | NITNWLWYIKLFIMIVGGLVGLRIVFAVLSIVNRVRQGYSPLSF | |
| | QTHLPTPRGPDRPEGIEEEGGERDRDRSIRLVNGSLALIWDDL | |
| | RSLCLFSYHRLRDLLLIVTRIVELLGRRGWEALKYWWNLLQY | |
| | WSQELKNSAVSLLNATAIAVAEGTDRVIEVVQGACRAIRHIPR | |
| | RIRQGLERILL | |
| 253 | AVGIGALFLGFLGAAGSTMGAASMTLTVQARQLLSDIVQQQN | HIV-1 NL4-3 Env |
| | NLLRAIEAQQHLLQLTVWGIKQLQARILAVERYLKDQQLLGI | (GP41) |
| | WGCSGKLICTTAVPWNASWSNKSLEQIWNNMTWMEWDREI | |
| | NNYTSLIHSLIEESQNQQEKNEQELLELDKWASLWNWFNITN | |
| | WLWYIKLFIMIVGGLVGLRIVFAVLSIVNRVRQGYSPLSFQTH | |
| | LPIPRGPDRPEGIEEEGGERGRDRSIRLVNGSLALIWDDLRSLC | |
| | LFSYHRLRDLLLIVTRIVELLGRRGWEALKYWWNLLQYWSQ | |
| | ELKNSAVNLLNATAIAVAEGTDRVIEVLQAAYRAIRHIPRRIR | |
| | QGLERILL | |
| 254 | KKINEGLLDSK | Cytoplasmic tail amino |
| | | acids |
| 255 | ILHYEKLSKIGLVKGITRKYKIKSNPLTKDIVIKMIPNVSNVSKC | Hendra virus F Protein, |
| | TGTVMENYKSRLTGILSPIKGAIELYNNNTHDLVGDVKLAGV | Without signal |
| | VMAGIAIGIATAAQITAGVALYEAMKNADNINKLKSSIESTNE | sequence |
| | AVVKLQETAEKTVYVLTALQDYINTNLVPTIDQISCKQTELAL | |
| | DLALSKYLSDLLFVFGPNLQDPVSNSMTIQAISQAFGGNYETL | |
| | LRTLGYATEDFDDLLESDSIAGQIVYVDLSSYYIIVRVYFPILTE | |
| | IQQAYVQELLPVSFNNDNSEWISIVPNFVLIRNTLISNIEVKYCL | |
| | ITKKSVICNQDYATPMTASVRECLTGSTDKCPRELVVSSHVPR | |
| | FALSGGVLFANCISVTCQCQTTGRAISQSGEQTLLMIDNTTCTT | |
| | VVLGNIIISLGKYLGSINYNSESIAVGPPVYTDKVDISSQISSMN | |
| | QSLQQSKDYIKEAQKILDTVNPSLISMLSMIILYVLSIAALCIGL | |
| | ITFISFVIVEKKRGNYSRLDDRQVRPVSNGDLYYIGT | |
| 256 | ILHYEKLSKIGLVKGVTRKYKIKSNPLTKDIVIKMIPNVSNMSQ | Nipah virus F Protein, |
| | CTGSVMENYKTRLNGILTPIKGALEIYKNNTHDLVGDVRLAG | without signal sequence |
| | VIMAGVAIGIATAAQITAGVALYEAMKNADNINKLKSSIESTN | |
| | EAVVKLQETAEKTVYVLTALQDYINTNLVPTIDKISCKQTELS | |
| | LDLALSKYLSDLLFVFGPNLQDPVSNSMTIQAISQAFGGNYET | |
| | LLRTLGYATEDFDDLLESDSITGQIIYVDLSSYYIIVRVYFPILTE | |
| | IQQAYIQELLPVSFNNDNSEWISIVPNFILVRNTLISNIEIGFCLIT | |
| | KRSVICNQDYATPMTNNMRECLTGSTEKCPRELVVSSHVPRF | |
| | ALSNGVLFANCISVTCQCQTTGRAISQSGEQTLLMIDNTTCPT | |
| | AVLGNVIISLGKYLGSVNYNSEGIAIGPPVFTDKVDISSQISSM | |
| | NQSLQQSKDYIKEAQRLLDTVNPSLISMLSMIILYVLSIASLCIG | |
| | LITFISFIIVEKKRNTYSRLEDRRVRPTSSGDLYYIGT | |
| 257 | TVLIIISYTLFYLNNAAIVGFDFDKLNKIGVVQGRVLNYKIKGD | Cedar Virus F Protein, |
| | PMTKDLVLKFIPNIVNITECVREPLSRYNETVRRLLLPIHNMLG | without signal sequence |
| | LYLNNTNAKMTGLMIAGVIMGGIAIGIATAAQITAGFALYEAK | |
| | KNTENIQKLTDSIMKTQDSIDKLTDSVGTSILILNKLQTYINNQ | |
| | LVPNLELLSCRQNKIEFDLMLTKYLVDLMTVIGPNINNPVNKD | |
| | MTIQSLSLLFDGNYDIMMSELGYTPQDFLDLIESKSITGQIIYV | |
| | DMENLYVVIRTYLPTLIEVPDAQIYEFNKITMSSNGGEYLSTIP | |
| | NFILIRGNYMSNIDVATCYMTKASVICNQDYSLPMSQNLRSCY | |
| | QGETEYCPVEAVIASHSPRFALTNGVIFANCINTICRCQDNGKT | |
| | ITQNINQFVSMIDNSTCNDVMVDKFTIKVGKYMGRKDINNINI | |
| | QIGPQIIIDKVDLSNEINKMNQSLKDSIFYLREAKRILDSVNISLI | |
| | SPSVQLFLIIISVLSFIILLIIIVYLYCKSKHSYKYNKFIDDPDYYN | |
| | DYKRERINGKASKSNNIYYVGD | |
| 258 | IHYDSLSKVGVIKGLTYNYKIKGSPSTKLMVVKLIPNIDSVKN | Mojiang virus, |
| | CTQKQYDEYKNLVRKALEPVKMAIDTMLNNVKSGNNKYRF | Tongguan 1 F Protein, |
| | AGAIMAGVALGVATAATVTAGIALHRSNENAQAIANMKSAIQ | without signal sequence |
| | NTNEAVKQLQLANKQTLAVIDTIRGEINNNIIPVINQLSCDTIG | |
| | LSVGIRLTQYYSEIITAFGPALQNPVNTRITIQAISSVFNGNFDE | |
| | LLKIMGYTSGDLYEILHSELIRGNIIDVDVDAGYIALEIEFPNLT | |
| | LVPNAVVQELMPISYNIDGDEWVTLVPRFVLTRTTLLSNIDTS | |
| | RCTITDSSVICDNDYALPMSHELIGCLQGDTSKCAREKVVSSY | |
| | VPKFALSDGLVYANCLNTICRCMDTDTPISQSLGATVSLLDNK | |
| | RCSVYQVGDVLISVGSYLGDGEYNADNVELGPPIVIDKIDIGN | |
| | QLAGINQTLQEAEDYIEKSEEFLKGVNPSIITLGSMVVLYIFMIL | |
| | IAIVSVIALVLSIKLTVKGNVVRQQFTYTQHVPSMENINYVSH | |
| 259 | SRALLRETDNYSNGLIVENLVRNCHHPSKNNLNYTKTQKRDS | Bat Paramyxovirus F |
| | TIPYRVEERKGHYPKIKHLIDKSYKHIKRGKRRNGHNGNIITIIL | Protein, without signal |
| | LLILILKTQMSEGAIHYETLSKIGLIKGITREYKVKGTPSSKDIVI | sequence |
| | KLIPNVTGLNKCTNISMENYKEQLDKILIPINNIIELYANSTKSA | |
| | PGNARFAGVIIAGVALGVAAAAQITAGIALHEARQNAERINLL | |
| | KDSISATNNAVAELQEATGGIVNVITGMQDYINTNLVPQIDKL | |
| | QCSQIKTALDISLSQYYSEILTVFGPNLQNPVTTSMSIQAISQSF | |
| | GGNIDLLLNLLGYTANDLLDLLESKSITGQITYINLEHYFMVIR | |
| | VYYPIMTTISNAYVQELIKISFNVDGSEWVSLVPSYILIRNSYLS | |
| | NIDISECLITKNSVICRHDFAMPMSYTLKECLTGDTEKCPREAV | |
| | VTSYVPRFAISGGVIYANCLSTTCQCYQTGKVIAQDGSQTLM | |
| | MIDNQTCSIVRIEEILISTGKYLGSQEYNTMHVSVGNPVFTDKL | |
| | DITSQISNINQSIEQSKFYLDKSKAILDKINLNLIGSVPISILFIIAI | |
| | LSLILSIITFVIVMIIVRRYNKYTPLINSDPSSRRSTIQDVYIIPNP | |
| | GEHSIRSAARSIDRDRD | |
| 260 | GYTFSNYW | CDR-H1 |
| 261 | ILPGSGST | CDR-H2 |
| 262 | ARRGYGYDEGFDY | CDR-H3 |
| 263 | QDINSY | CDR-L1 |
| 264 | RAN | CDR-L2 |
| 265 | LQYDEFPPT | CDR-L3 |
| 266 | NYWIE | CDR-H1 |
| 267 | EILPGSGSTSYNEKFKG | CDR-H2 |
| 268 | RGYGYDEGFDY | CDR-H3 |
| 269 | KASQDINSYLS | CDR-L1 |
| 270 | RANRLVD | CDR-L2 |
| 271 | GYTFSNY | CDR-H1 |
| 272 | LPGSGS | CDR-H2 |
| 273 | QVQLQQSGAELMKPGASVKMSCKATGYTFSNYWIEWVKQRP | VH |
| | GHGLEWIGEILPGSGSTSYNEKFKGKATFTADTSSSTAYMQLS | |
| | SLTSEDSAVYYCARRGYGYDEGFDYWGQGSTLTVSS | |
| 274 | DIKMTQSPSSMYASLGERVTITCKASQDINSYLSWFQQKPGKS | VL |
| | PKTLIYRANRLVDGVPSRFSGSGSGQDYSLTISSLEYEDMGIYY | |
| | CLQYDEFPPTFGAGTKLELKR | |
| 275 | GGGGSGGGGSGGGGS | linker |
| 276 | QVQLQQSGAELMKPGASVKMSCKATGYTFSNYWIEWVKQRP | scFv |
| | GHGLEWIGEILPGSGSTSYNEKFKGKATFTADTSSSTAYMQLS | |
| | SLTSEDSAVYYCARRGYGYDEGFDYWGQGSTLTVSSGGGGS | |
| | GGGGSGGGGSDIKMTQSPSSMYASLGERVTITCKASQDINSYL | |
| | SWFQQKPGKSPKTLIYRANRLVDGVPSRFSGSGSGQDYSLTIS | |
| | SLEYEDMGIYYCLQYDEFPPTFGAGTKLELKR | |
| 277 | GYTFTDYV | CDR-H1 |
| 278 | IYPGSGSS | CDR-H2 |
| 279 | ARPGDLGFAY | CDR-H3 |
| 280 | QSVDYDGDSY | CDR-L1 |
| 281 | AAS | CDR-L2 |
| 282 | QQSNKDPFT | CDR-L3 |
| 283 | DYVIS | CDR-H1 |
| 284 | EIYPGSGSSYYNEKFKG | CDR-H2 |
| 285 | PGDLGFAY | CDR-H3 |
| 286 | KASQSVDYDGDSYMN | CDR-L1 |
| 287 | AASNLES | CDR-L2 |
| 288 | QQSNKDPFT | CDR-L3 |
| 289 | GYTFTDY | CDR-H1 |
| 290 | YPGSGS | CDR-H2 |
| 291 | QVQLQQSGPELVKPGASVKMSCKASGYTFTDYVISWVRQAP | VH |
| | GQGLEWIGEIYPGSGSSYYNEKFKGRATLTADKSSNTAYMQL | |
| | SSLRSEDSAVYFCARPGDLGFAYWGQGTLVTVSS | |
| 292 | DIVLTQSPSSLAVSLGQRATISCKASQSVDYDGDSYMNWYQQ | VL |
| | KPGQPPKLLIYAASNLESGIPARFSGSGSGTDFTLTIHPVEEEDA | |
| | ATYYCQQSNKDPFTFGGGTKLELKR | |
| 293 | QVQLQQSGPELVKPGASVKMSCKASGYTFTDYVISWVRQAP | scFv |
| | GQGLEWIGEIYPGSGSSYYNEKFKGRATLTADKSSNTAYMQL | |
| | SSLRSEDSAVYFCARPGDLGFAYWGQGTLVTVSSGGGGSGGG | |
| | GSGGGGSDIVLTQSPSSLAVSLGQRATISCKASQSVDYDGDSY | |
| | MNWYQQKPGQPPKLLIYAASNLESGIPARFSGSGSGTDFTLTI | |
| | HPVEEEDAATYYCQQSNKDPFTFGGGTKLELKR | |
| 294 | GGTFSSYA | CDR-H1 |
| 295 | INPNSGGT | CDR-H2 |
| 296 | ARDGYSGSYSD | CDR-H3 |
| 297 | QSVLSSSYNKNY | CDR-L1 |
| 298 | WAS | CDR-L2 |
| 299 | QQYYSTPWT | CDR-L3 |
| 300 | SYAIS | CDR-H1 |
| 301 | WINPNSGGTNYAQKFQG | CDR-H2 |
| 302 | DGYSGSYSD | CDR-H3 |
| 303 | KSSQSVLSSSYNKNYLA | CDR-L1 |
| 304 | WASTRES | CDR-L2 |
| 305 | GGTFSSY | CDR-H1 |
| 306 | NPNSGG | CDR-H2 |
| 307 | QVQLVQSGAEVKKPGASVKVSCKASGGTFSSYAISWVRQAPG | VH |
| | QGLEWMGWINPNSGGTNYAQKFQGRVTMTRDTSTSTVYME | |
| | LSSLRSEDTAVYYCARDGYSGSYSDWGQGTLVTVSS | |
| 308 | DIVMTQSPDSLAVSLGERATINCKSSQSVLSSSYNKNYLAWY | VL |
| | QQKPGQPPKLLIYWASTRESGVPDRFSGSGSGTDFTLTISSLQA | |
| | EDVAVYYCQQYYSTPWTFGQGTKVEIK | |
| 309 | QVQLVQSGAEVKKPGASVKVSCKASGGTFSSYAISWVRQAPG | scFv |
| | QGLEWMGWINPNSGGTNYAQKFQGRVTMTRDTSTSTVYME | |
| | LSSLRSEDTAVYYCARDGYSGSYSDWGQGTLVTVSSGGGGS | |
| | GGGGSGGGGSDIVMTQSPDSLAVSLGERATINCKSSQSVLSSS | |
| | YNKNYLAWYQQKPGQPPKLLIYWASTRESGVPDRFSGSGSGT | |
| | DFTLTISSLQAEDVAVYYCQQYYSTPWTFGQGTKVEIK | |
| 310 | GYTFTSYD | CDR-H1 |
| 311 | IIPLSGAP | CDR-H2 |
| 312 | ARGALYNWNDGWFDP | CDR-H3 |
| 313 | QDIGDY | CDR-L1 |
| 314 | DAS | CDR-L2 |
| 315 | QQANSFPLT | CDR-L3 |
| 316 | SYDIN | CDR-H1 |
| 317 | GIIPLSGAPNYAHKFQG | CDR-H2 |
| 318 | GALYNWNDGWFDP | CDR-H3 |
| 319 | RASQDIGDYLA | CDR-L1 |
| 320 | DASSLQS | CDR-L2 |
| 321 | GYTFTSY | CDR-H1 |
| 322 | IPLSGA | CDR-H2 |
| 323 | QVQLVQSGAEVKKPGSSVKVSCKASGYTFTSYDINWVRQAP | VH |
| | GQGLEWMGGIIPLSGAPNYAHKFQGRVTITADESTSTAYMEL | |
| | SSLRSEDTAVYYCARGALYNWNDGWFDPWGQGTLVTVSS | |
| 324 | DIQMTQSPSSLSASVGDRVTITCRASQDIGDYLAWYQQKPGK | VL |
| | APKLLIYDASSLQSGVPSRFSGSGSGTDFTLTISSLQPEDFATYY | |
| | CQQANSFPLTFGGGTKVEIK | |
| 325 | QVQLVQSGAEVKKPGSSVKVSCKASGYTFTSYDINWVRQAP | scFv |
| | GQGLEWMGGIIPLSGAPNYAHKFQGRVTITADESTSTAYMEL | |
| | SSLRSEDTAVYYCARGALYNWNDGWFDPWGQGTLVTVSSG | |
| | GGGSGGGGSGGGGSDIQMTQSPSSLSASVGDRVTITCRASQDI | |
| | GDYLAWYQQKPGKAPKLLIYDASSLQSGVPSRFSGSGSGTDF | |
| | TLTISSLQPEDFATYYCQQANSFPLTFGGGTKVEIK | |
| 326 | GDTFSDHA | CDR-H1 |
| 327 | MNPKIGNT | CDR-H2 |
| 328 | VYDSSGYDAFDI | CDR-H3 |
| 329 | QSVLSTSYNRNF | CDR-L1 |
| 330 | WAS | CDR-L2 |
| 331 | QQYYSTPYT | CDR-L3 |
| 332 | DHAIN | CDR-H1 |
| 333 | WMNPKIGNTGYAQKFQG | CDR-H2 |
| 334 | DSSGYDAFDI | CDR-H3 |
| 335 | KSSQSVLSTSYNRNFLA | CDR-L1 |
| 336 | WASTRQS | CDR-L2 |
| 337 | GDTFSDH | CDR-H1 |
| 338 | NPKIGN | CDR-H2 |
| 339 | QVQLVQSGAEVKKPGASVKVSCKASGDTFSDHAINWVRQAP | VH |
| | GQGLEWMGWMNPKIGNTGYAQKFQGRVTMTRDTSTSTVYM | |
| | ELSSLRSEDTAVYYCVYDSSGYDAFDIWGQGTTVTVSS | |
| 340 | DIVMTQSPDSLAVSLGERATINCKSSQSVLSTSYNRNFLAWYQ | VL |
| | QKPGQPPKLLIYWASTRQSGVPDRFSGSGSGTDFTLTISSLQAE | |
| | DVAVYYCQQYYSTPYTFGQGTKLEIK | |
| 341 | QVQLVQSGAEVKKPGASVKVSCKASGDTFSDHAINWVRQAP | scFv |
| | GQGLEWMGWMNPKIGNTGYAQKFQGRVTMTRDTSTSTVYM | |
| | ELSSLRSEDTAVYYCVYDSSGYDAFDIWGQGTTVTVSSGGGG | |
| | SGGGGSGGGGSDIVMTQSPDSLAVSLGERATINCKSSQSVLST | |
| | SYNRNFLAWYQQKPGQPPKLLIYWASTRQSGVPDRFSGSGSG | |
| | TDFTLTISSLQAEDVAVYYCQQYYSTPYTFGQGTKLEIK | |
| 342 | GYSLITHW | CDR-H1 |
| 343 | INPSDGVT | CDR-H2 |
| 344 | AREYYGEGFDY | CDR-H3 |
| 345 | QGISNY | CDR-L1 |
| 346 | SAS | CDR-L2 |
| 347 | QQSYSTPLT | CDR-L3 |
| 348 | THWMH | CDR-H1 |
| 349 | MINPSDGVTYYAQTFQG | CDR-H2 |
| 350 | EYYGEGFDY | CDR-H3 |
| 351 | RASQGISNYLA | CDR-L1 |
| 352 | SASNLQS | CDR-L2 |
| 353 | GYSLITH | CDR-H1 |
| 354 | NPSDGV | CDR-H2 |
| 355 | QVQLVQSGAEVKKPGASVKVSCKASGYSLITHWMHWVRQA | VH |
| | PGQGLEWMGMINPSDGVTYYAQTFQGRVTMTRDTSTSTVYM | |
| | ELSSLRSEDTAVYYCAREYYGEGFDYWGQGTLVTVSS | |
| 356 | DIQMTQSPSSLSASVGDRVTITCRASQGISNYLAWYQQKPGKA | VL |
| | PKLLIYSASNLQSGVPSRFSGSGSGTDFTLTISSLQPEDFATYYC | |
| | QQSYSTPLTFGGGTKVEIKR | |
| 357 | QVQLVQSGAEVKKPGASVKVSCKASGYSLITHWMHWVRQA | scFv |
| | PGQGLEWMGMINPSDGVTYYAQTFQGRVTMTRDTSTSTVYM | |
| | ELSSLRSEDTAVYYCAREYYGEGFDYWGQGTLVTVSSGGGG | |
| | SGGGGSGGGGSDIQMTQSPSSLSASVGDRVTITCRASQGISNY | |
| | LAWYQQKPGKAPKLLIYSASNLQSGVPSRFSGSGSGTDFTLTI | |
| | SSLQPEDFATYYCQQSYSTPLTFGGGTKVEIKR | |
| 358 | GFTFSGYW | CDR-H1 |
| 359 | ISPGGGST | CDR-H2 |
| 360 | ASSLTATHTYEYDY | CDR-H3 |
| 361 | GYWMY | CDR-H1 |
| 362 | AISPGGGSTYYPDSVKG | CDR-H2 |
| 363 | SLTATHTYEYDY | CDR-H3 |
| 364 | GFTFSGY | CDR-H1 |
| 365 | SPGGGS | CDR-H2 |
| 366 | EVQLVESGGGLVQSGGSLRLSCAASGFTFSGYWMYWVRQAP | VHH |
| | GKGLEWVSAISPGGGSTYYPDSVKGRFTISRDNAKNTLYLQM | |
| | NSLEPEDTALYYCASSLTATHTYEYDYWGQGTQVTVSS | |
| 367 | TTTPAPRPPTPAPTIASQPLSLRPEACRPAAGGAVHTRGLDFAC | CD8α hinge domain |
| | D | |
| 368 | IYIWAPLAGTCGVLLLSLVITLYC | CD8α transmembrane |
| | | domain |
| 369 | QVQLVQSGAEVKKPGASVKVSCKASGGTFSSYAISWVRQAPG | CD8_1 VH |
| | QGLEWMGIIDPSDGNTNYAQNFQGRVTMTRDTSTSTVYMELS | |
| | SLRSEDTAVYYCAKERAAAGYYYYMDVWGQGTTVTVSS | |
| 370 | DIQMTQSPSSLSASVGDRVTITCRASQSISSYLNWYQQKPGKA | CD8_1 VL |
| | PKLLIYAASSLQSGVPSRFSGSGSGTDFTLTISSLQPEDFATYYC | |
| | QQSYSTPLTFGGGTKVEIKR | |
| 371 | QVQLVQSGAEVKKPGASVKVSCKASGYTFTDYYIQWVRQAP | CD8_2 VH |
| | GQGLEWMGWINPNSGGTSYAQKFQGRVTMTRDTSTSTVYME | |
| | LSSLRSEDTAVYYCAKEGDYYYGMDAWGQGTMVTVSS | |
| 372 | DIVMTQSPLSLPVTPGEPASISCRSSQSLLHSNGYNYLDWYLQ | CD8_2 VL |
| | KPGQSPQLLIYLGSNRASGVPDRFSGSGSGTDFTLKISRVEAED | |
| | VGVYYCMQGLQTPHTFGQGTKVEIKR | |
| 373 | QVQLVQSGAEVKKPGASVKVSCKASGYTFTSYYMHWVRQA | CD8_3 VH |
| | PGQGLEWMGGFDPEDGETIYAQKFQGRVTMTRDTSTSTVYM | |
| | ELSSLRSEDTAVYYCARDQGWGMDVWGQGTTVTVSS | |
| 374 | DIQMTQSPSSLSASVGDRVTITCRASQSISSYLNWYQQKPGKA | CD8_3 VL |
| | PKLLIYAASSLQSGVPSRFSGSGSGTDFTLTISSLQPEDFATYYC | |
| | QQTYSTPYTFGQGTKLEIKR | |
| 375 | QVQLVQSGAEVKKPGASVKVSCKASGYTFTNHYMHWVRQA | CD8_4 VH |
| | PGQGLEWMGWMNPNSGNTGYAQKFQGRVTMTRDTSTSTVY | |
| | MELSSLRSEDTAVYYCASSESGSDLDYWGQGTLVTVSS | |
| 376 | DIQMTQSPSSLSASVGDRVTITCRASQTIGNYVNWYQQKPGK | CD8_4 VL |
| | APKLLIYGASNLHTGVPSRFSGSGSGTDFTLTISSLQPEDFATY | |
| | YCQQTYSAPLTFGGGTKVEIKR | |
| 377 | QVQLVESGGGLVQAGGSLRLSCAASGRTFSGYVMGWFRQAP | VHH |
| | GKQRKFVAAISRGGLSTSYADSVKGRFTISRDNAKNTVFLQM | |
| | NTLKPEDTAVYYCAADRSDLYEITAASNIDSWGQGTLVTVSS | |
| 378 | MALPVTALLLPLALLLHAARP | CD8α signal peptide |
| 379 | METDTLLLWVLLLWVPGSTG | IgK signal peptide |
| 380 | MLLLVTSLLLCELPHPAFLLIP | GMCSFR-α (CSF2RA) |
| | | signal peptide |
| 381 | TTTPAPRPPTPAPTIASQPLSLRPEACRPAAGGAVHTRGLDFAC | CD8α hinge domain |
| | D | |
| 382 | IEVMYPPPYLDNEKSNGTIIHVKGKHLCPSPLFPGPSKP | CD28 hinge domain |
| 383 | AAAIEVMYPPPYLDNEKSNGTIIHVKGKHLCPSPLFPGPSKP | CD28 hinge domain |
| 384 | ESKYGPPCPPCP | IgG4 hinge domain |
| 385 | ESKYGPPCPSCP | IgG4 hinge domain |
| 386 | ESKYGPPCPPCPAPEFLGGPSVFLFPPKPKDTLMISRTPEVTCV | IgG4 hinge-CH2-CH3 |
| | VVDVSQEDPEVQFNWYVDGVEVHNAKTKPREEQFNSTYRVV | domain |
| | SVLTVLHQDWLNGKEYKCKVSNKGLPSSIEKTISKAKGQPREP | |
| | QVYTLPPSQEEMTKNQVSLTCLVKGFYPSDIAVEWESNGQPE | |
| | NNYKTTPPVLDSDGSFFLYSRLTVDKSRWQEGNVFSCSVMHE | |
| | ALHNHYTQKSLSLSLGK | |
| 387 | IYIWAPLAGTCGVLLLSLVITLYC | CD8α transmembrane |
| | | domain |
| 388 | FWVLVVVGGVLACYSLLVTVAFIIFWV | CD28 transmembrane |
| | | domain |
| 389 | MFWVLVVVGGVLACYSLLVTVAFIIFWV | CD28 transmembrane |
| | | domain |
| 390 | KRGRKKLLYIFKQPFMRPVQTTQEEDGCSCRFPEEEEGGCEL | 4-1BB costimulatory |
| | | domain |
| 391 | RSKRSRLLHSDYMNMTPRRPGPTRKHYQPYAPPRDFAAYRS | CD28 costimulatory |
| | | domain |
| 392 | RVKFSRSADAPAYQQGQNQLYNELNLGRREEYDVLDKRRGR | CD3 signaling domain |
| | DPEMGGKPRRKNPQEGLYNELQKDKMAEAYSEIGMKGERRR | |
| | GKGHDGLYQGLSTATKDTYDALHMQALPPR | |
| 393 | RVKFSRSADAPAYKQGQNQLYNELNLGRREEYDVLDKRRGR | CD3ζ signaling domain |
| | DPEMGGKPRRKNPQEGLYNELQKDKMAEAYSEIGMKGERRR | (with Q to K mutation |
| | GKGHDGLYQGLSTATKDTYDALHMQALPPR | at position 14) |
| 394 | DIQMTQTTSSLSASLGDRVTISCRASQDISKYLNWYQQKPDGT | Anti-CD19 FMC63 |
| | VKLLIYHTSRLHSGVPSRFSGSGSGTDYSLTISNLEQEDIATYF | scFv entire sequence, |
| | CQQGNTLPYTFGGGTKLEITGSTSGSGKPGSGEGSTKGEVKLQ | with Whitlow linker |
| | ESGPGLVAPSQSLSVTCTVSGVSLPDYGVSWIRQPPRKGLEWL | |
| | GVIWGSETTYYNSALKSRLTIIKDNSKSQVFLKMNSLQTDDTA | |
| | IYYCAKHYYYGGSYAMDYWGQGTSVTVSS | |
| 395 | DIQMTQTTSSLSASLGDRVTISCRASQDISKYLNWYQQKPDGT | Anti-CD19 FMC63 |
| | VKLLIYHTSRLHSGVPSRFSGSGSGTDYSLTISNLEQEDIATYF | scFv light chain |
| | CQQGNTLPYTFGGGTKLEIT | variable region |
| 396 | QDISKY | Anti-CD19 FMC63 |
| | | scFv light chain CDR1 |
| 397 | HTS | Anti-CD19 FMC63 |
| | | scFv light chain CDR2 |
| 398 | QQGNTLPYT | Anti-CD19 FMC63 |
| | | scFv light chain CDR3 |
| 399 | GSTSGSGKPGSGEGSTKG | Whitlow linker |
| 400 | EVKLQESGPGLVAPSQSLSVTCTVSGVSLPDYGVSWIRQPPRK | Anti-CD19 FMC63 |
| | GLEWLGVIWGSETTYYNSALKSRLTIIKDNSKSQVFLKMNSLQ | scFv heavy chain |
| | TDDTAIYYCAKHYYYGGSYAMDYWGQGTSVTVSS | variable region |
| 401 | GVSLPDYG | Anti-CD19 FMC63 |
| | | scFv heavy chain |
| | | CDR1 |
| 402 | IWGSETT | Anti-CD19 FMC63 |
| | | scFv heavy chain |
| | | CDR2 |
| 403 | AKHYYYGGSYAMDY | Anti-CD19 FMC63 |
| | | scFv heavy chain |
| | | CDR3 |
| 404 | DIQMTQTTSSLSASLGDRVTISCRASQDISKYLNWYQQKPDGT | Anti-CD19 FMC63 |
| | VKLLIYHTSRLHSGVPSRFSGSGSGTDYSLTISNLEQEDIATYF | scFv entire sequence, |
| | CQQGNTLPYTFGGGTKLEITGGGGSGGGGSGGGGSEVKLQES | with 3xG₄S linker |
| | GPGLVAPSQSLSVTCTVSGVSLPDYGVSWIRQPPRKGLEWLG | |
| | VIWGSETTYYNSALKSRLTIIKDNSKSQVFLKMNSLQTDDTAI | |
| | YYCAKHYYYGGSYAMDYWGQGTSVTVSS | |
| 405 | GGGGSGGGGSGGGGS | 3xG₄S linker |
| 406 | atggccttaccagtgaccgccttgctcctgccgctggccttgctgctccacgccgccaggccgga | Exemplary CD19 CAR |
| | catccagatgacacagactacatcctccctgtctgcctctctgggagacagagtcaccatcagttgc | nucleotide sequence |
| | agggcaagtcaggacattagtaaatatttaaattggtatcagcagaaaccagatggaactgttaaac | |
| | tcctgatctaccatacatcaagattacactcaggagtcccatcaaggttcagtggcagtgggtctgg | |
| | aacagattattctctcaccattagcaacctggagcaagaagatattgccacttacttttgccaacagg | |
| | gtaatacgcttccgtacacgttcggaggggggaccaagctggagatcacaggctccacctctgga | |
| | tccggcaagcccggatctggcgagggatccaccaagggcgaggtgaaactgcaggagtcagg | |
| | acctggcctggtggcgccctcacagagcctgtccgtcacatgcactgtctcaggggtctcattacc | |
| | cgactatggtgtaagctggattcgccagcctccacgaaagggtctggagtggctgggagtaatat | |
| | ggggtagtgaaaccacatactataattcagctctcaaatccagactgaccatcatcaaggacaactc | |
| | caagagccaagttttcttaaaaatgaacagtctgcaaactgatgacacagccatttactactgtgcca | |
| | aacattattactacggtggtagctatgctatggactactggggccaaggaacctcagtcaccgtctc | |
| | ctcaaccacgacgccagcgccgcgaccaccaacaccggcgcccaccatcgcgtcgcagcccct | |
| | gtccctgcgcccagaggcgtgccggccagcggcggggggcgcagtgcacacgagggggctg | |
| | gacttcgcctgtgatatctacatctgggcgcccttggccgggacttgtggggtccttctcctgtcact | |
| | ggttatcaccctttactgcaaacggggcagaaagaaactcctgtatatattcaaacaaccatttatga | |
| | gaccagtacaaactactcaagaggaagatggctgtagctgccgatttccagaagaagaagaagg | |
| | aggatgtgaactgagagtgaagttcagcaggagcgcagacgcccccgcgtaccagcagggcca | |
| | gaaccagctctataacgagctcaatctaggacgaagagaggagtacgatgttttggacaagagac | |
| | gtggccgggaccctgagatggggggaaagccgagaaggaagaaccctcaggaaggcctgtac | |
| | aatgaactgcagaaagataagatggcggaggcctacagtgagattgggatgaaaggcgagcgc | |
| | cggaggggcaaggggcacgatggcctttaccagggtctcagtacagccaccaaggacacctac | |
| | gacgcccttcacatgcaggccctgccccctcgc | |
| 407 | MALPVTALLLPLALLLHAARPDIQMTQTTSSLSASLGDRVTIS | Exemplary CD19 CAR |
| | CRASQDISKYLNWYQQKPDGTVKLLIYHTSRLHSGVPSRFSGS | amino acid sequence |
| | GSGTDYSLTISNLEQEDIATYFCQQGNTLPYTFGGGTKLEITGS | |
| | TSGSGKPGSGEGSTKGEVKLQESGPGLVAPSQSLSVTCTVSGV | |
| | SLPDYGVSWIRQPPRKGLEWLGVIWGSETTYYNSALKSRLTII | |
| | KDNSKSQVFLKMNSLQTDDTAIYYCAKHYYYGGSYAMDYW | |
| | GQGTSVTVSSTTTPAPRPPTPAPTIASQPLSLRPEACRPAAGGA | |
| | VHTRGLDFACDIYIWAPLAGTCGVLLLSLVITLYCKRGRKKLL | |
| | YIFKQPFMRPVQTTQEEDGCSCRFPEEEEGGCELRVKFSRSAD | |
| | APAYQQGQNQLYNELNLGRREEYDVLDKRRGRDPEMGGKPR | |
| | RKNPQEGLYNELQKDKMAEAYSEIGMKGERRRGKGHDGLY | |
| | QGLSTATKDTYDALHMQALPPR | |
| 408 | atggccttaccagtgaccgccttgctcctgccgctggccttgctgctccacgccgccaggccgga | Tisagenlecleucel CD19 |
| | catccagatgacacagactacatcctccctgtctgcctctctgggagacagagtcaccatcagttgc | CAR nucleotide |
| | agggcaagtcaggacattagtaaatatttaaattggtatcagcagaaaccagatggaactgttaaac | sequence |
| | tcctgattaccatacatcaagattacactcaggagtcccatcaaggttcagtggcagtgggtctgg | |
| | aacagattattctctcaccattagcaacctggagcaagaagatattgccacttacttttgccaacagg | |
| | gtaatacgcttccgtacacgttcggaggggggaccaagctggagatcacaggtggcggtggctc | |
| | gggcggtggtgggtcgggtggcggcggatctgaggtgaaactgcaggagtcaggacctggcct | |
| | ggtggcgccctcacagagcctgtccgtcacatgcactgtctcaggggtctcattacccgactatgg | |
| | tgtaagctggattcgccagcctccacgaaagggtctggagtggctgggagtaatatggggtagtg | |
| | aaaccacatactataattcagctctcaaatccagactgaccatcatcaaggacaactccaagagcc | |
| | aagttttcttaaaaatgaacagtctgcaaactgatgacacagccatttactactgtgccaaacattatt | |
| | actacggtggtagctatgctatggactactggggccaaggaacctcagtcaccgtctcctcaacca | |
| | cgacgccagcgccgcgaccaccaacaccggcgcccaccatcgcgtcgcagcccctgtccctgc | |
| | gcccagaggcgtgccggccagcggggggggcgcagtgcacacgagggggctggacttcgc | |
| | ctgtgatatctacatctgggcgcccttggccgggacttgtggggtccttctcctgtcactggttatcac | |
| | cctttactgcaaacggggcagaaagaaactcctgtatatattcaaacaaccatttatgagaccagta | |
| | caaactactcaagaggaagatggctgtagctgccgatttccagaagaagaagaaggaggatgtg | |
| | aactgagagtgaagttcagcaggagcgcagacgcccccgcgtacaagcagggccagaaccag | |
| | ctctataacgagctcaatctaggacgaagagaggagtacgatgttttggacaagagacgtggccg | |
| | ggaccctgagatggggggaaagccgagaaggaagaaccctcaggaaggcctgtacaatgaact | |
| | gcagaaagataagatggcggaggcctacagtgagattgggatgaaaggcgagcgccggaggg | |
| | gcaaggggcacgatggcctttaccagggtctcagtacagccaccaaggacacctacgacgccct | |
| | tcacatgcaggccctgccccctegc | |
| 409 | MALPVTALLLPLALLLHAARPDIQMTQTTSSLSASLGDRVTIS | Tisagenlecleucel CD19 |
| | CRASQDISKYLNWYQQKPDGTVKLLIYHTSRLHSGVPSRFSGS | CAR amino acid |
| | GSGTDYSLTISNLEQEDIATYFCQQGNTLPYTFGGGTKLEITGG | sequence |
| | GGSGGGGSGGGGSEVKLQESGPGLVAPSQSLSVTCTVSGVSL | |
| | PDYGVSWIRQPPRKGLEWLGVIWGSETTYYNSALKSRLTIIKD | |
| | NSKSQVFLKMNSLQTDDTAIYYCAKHYYYGGSYAMDYWGQ | |
| | GTSVTVSSTTTPAPRPPTPAPTIASQPLSLRPEACRPAAGGAVH | |
| | TRGLDFACDIYIWAPLAGTCGVLLLSLVITLYCKRGRKKLLYI | |
| | FKQPFMRPVQTTQEEDGCSCRFPEEEEGGCELRVKFSRSADAP | |
| | AYKQGQNQLYNELNLGRREEYDVLDKRRGRDPEMGGKPRR | |
| | KNPQEGLYNELQKDKMAEAYSEIGMKGERRRGKGHDGLYQ | |
| | GLSTATKDTYDALHMQALPPR | |
| 410 | atgctgctgctggtgaccagcctgctgctgtgcgagctgccccaccccgcctttctgctgatcccc | Lisocabtagene |
| | gacatccagatgacccagaccacctccagcctgagcgccagcctgggcgaccgggtgaccatc | maraleucel CD19 CAR |
| | agctgccgggccagccaggacatcagcaagtacctgaactggtatcagcagaagcccgacggc | nucleotide sequence |
| | accgtcaagctgctgatctaccacaccagccggctgcacagcggcgtgcccagccggtttagcg | |
| | gcagcggctccggcaccgactacagcctgaccatctccaacctggaacaggaagatatcgccac | |
| | ctacttttgccagcagggcaacacactgccctacacctttggcggcggaacaaagctggaaatca | |
| | ccggcagcacctccggcagcggcaagcctggcagcggcgagggcagcaccaagggcgaggt | |
| | gaagctgcaggaaagcggccctggcctggtggcccccagccagagcctgagcgtgacctgcac | |
| | cgtgagcggcgtgagcctgcccgactacggcgtgagctggatccggcagccccccaggaagg | |
| | gcctggaatggctgggcgtgatctggggcagcgagaccacctactacaacagcgccctgaaga | |
| | gccggctgaccatcatcaaggacaacagcaagagccaggtgttcctgaagatgaacagcctgca | |
| | gaccgacgacaccgccatctactactgcgccaagcactactactacggcggcagctacgccatg | |
| | gactactggggccagggcaccagcgtgaccgtgagcagcgaatctaagtacggaccgccctgc | |
| | cccccttgccctatgttctgggtgctggtggtggtcggaggcgtgctggcctgctacagcctgctg | |
| | gtcaccgtggccttcatcatcttttgggtgaaacggggcagaaagaaactcctgtatatattcaaaca | |
| | accatttatgagaccagtacaaactactcaagaggaagatggctgtagctgccgatttccagaaga | |
| | agaagaaggaggatgtgaactgcgggtgaagttcagcagaagcgccgacgcccctgcctacca | |
| | gcagggccagaatcagctgtacaacgagctgaacctgggcagaagggaagagtacgacgtcct | |
| | ggataagcggagaggccgggaccctgagatgggcggcaagcctcggcggaagaacccccag | |
| | gaaggcctgtataacgaactgcagaaagacaagatggccgaggcctacagcgagatcggcatg | |
| | aagggcgagcggaggcggggcaagggccacgacggcctgtatcagggcctgtccaccgcca | |
| | ccaaggatacctacgacgccctgcacatgcaggccctgcccccaagg | |
| 411 | MLLLVTSLLLCELPHPAFLLIPDIQMTQTTSSLSASLGDRVTISC | Lisocabtagene |
| | RASQDISKYLNWYQQKPDGTVKLLIYHTSRLHSGVPSRFSGSG | maraleucel CD19 CAR |
| | SGTDYSLTISNLEQEDIATYFCQQGNTLPYTFGGGTKLEITGST | amino acid sequence |
| | SGSGKPGSGEGSTKGEVKLQESGPGLVAPSQSLSVTCTVSGVS | |
| | LPDYGVSWIRQPPRKGLEWLGVIWGSETTYYNSALKSRLTIIK | |
| | DNSKSQVFLKMNSLQTDDTAIYYCAKHYYYGGSYAMDYWG | |
| | QGTSVTVSSESKYGPPCPPCPMFWVLVVVGGVLACYSLLVTV | |
| | AFIIFWVKRGRKKLLYIFKQPFMRPVQTTQEEDGCSCRFPEEEE | |
| | GGCELRVKFSRSADAPAYQQGQNQLYNELNLGRREEYDVLD | |
| | KRRGRDPEMGGKPRRKNPQEGLYNELQKDKMAEAYSEIGMK | |
| | GERRRGKGHDGLYQGLSTATKDTYDALHMQALPPR | |
| 412 | atgcttctcctggtgacaagccttctgctctgtgagttaccacacccagcattcctcctgatcccaga | Axicabtagene ciloleucel |
| | catccagatgacacagactacatcctccctgtctgcctctctgggagacagagtcaccatcagttgc | CD19 CAR nucleotide |
| | agggcaagtcaggacattagtaaatatttaaattggtatcagcagaaaccagatggaactgttaaac | sequence |
| | tcctgatctaccatacatcaagattacactcaggagtcccatcaaggttcagtggcagtgggtctgg | |
| | aacagattattctctcaccattagcaacctggagcaagaagatattgccacttacttttgccaacagg | |
| | gtaatacgcttccgtacacgttcggaggggggactaagttggaaataacaggctccacctctggat | |
| | ccggcaagcccggatctggcgagggatccaccaagggcgaggtgaaactgcaggagtcagga | |
| | cctggcctggtggcgccctcacagagcctgtccgtcacatgcactgtctcaggggtctcattaccc | |
| | gactatggtgtaagctggattcgccagcctccacgaaagggtctggagtggctgggagtaatatg | |
| | gggtagtgaaaccacatactataattcagctctcaaatccagactgaccatcatcaaggacaactcc | |
| | aagagccaagttttcttaaaaatgaacagtctgcaaactgatgacacagccatttactactgtgccaa | |
| | acattattactacggtggtagctatgctatggactactggggtcaaggaacctcagtcaccgtctcct | |
| | cagcggccgcaattgaagttatgtatcctcctccttacctagacaatgagaagagcaatggaaccat | |
| | tatccatgtgaaagggaaacacctttgtccaagtcccctatttcccggaccttctaagcccttttgggt | |
| | gctggtggtggttgggggagtcctggcttgctatagcttgctagtaacagtggcctttattattttctg | |
| | ggtgaggagtaagaggagcaggctcctgcacagtgactacatgaacatgactccccgccgcccc | |
| | gggcccacccgcaagcattaccagccctatgccccaccacgcgacttcgcagcctatcgctcca | |
| | gagtgaagttcagcaggagcgcagacgcccccgcgtaccagcagggccagaaccagctctata | |
| | acgagctcaatctaggacgaagagaggagtacgatgttttggacaagagacgtggccgggaccc | |
| | tgagatggggggaaagccgagaaggaagaaccctcaggaaggcctgtacaatgaactgcagaa | |
| | agataagatggcggaggcctacagtgagattgggatgaaaggcgagcgccggaggggcaagg | |
| | ggcacgatggcctttaccagggtctcagtacagccaccaaggacacctacgacgcccttcacatg | |
| | caggccctgccccctcgc | |
| 413 | MLLLVTSLLLCELPHPAFLLIPDIQMTQTTSSLSASLGDRVTISC | Axicabtagene ciloleucel |
| | RASQDISKYLNWYQQKPDGTVKLLIYHTSRLHSGVPSRFSGSG | CD19 CAR amino acid |
| | SGTDYSLTISNLEQEDIATYFCQQGNTLPYTFGGGTKLEITGST | sequence |
| | SGSGKPGSGEGSTKGEVKLQESGPGLVAPSQSLSVTCTVSGVS | |
| | LPDYGVSWIRQPPRKGLEWLGVIWGSETTYYNSALKSRLTIIK | |
| | DNSKSQVFLKMNSLQTDDTAIYYCAKHYYYGGSYAMDYWG | |
| | QGTSVTVSSAAAIEVMYPPPYLDNEKSNGTIIHVKGKHLCPSP | |
| | LFPGPSKPFWVLVVVGGVLACYSLLVTVAFIIFWVRSKRSRLL | |
| | HSDYMNMTPRRPGPTRKHYQPYAPPRDFAAYRSRVKFSRSA | |
| | DAPAYQQGQNQLYNELNLGRREEYDVLDKRRGRDPEMGGK | |
| | PRRKNPQEGLYNELQKDKMAEAYSEIGMKGERRRGKGHDGL | |
| | YQGLSTATKDTYDALHMQALPPR | |
| 414 | DIVLTQSPAILSASPGEKVTMTCRASSSVNYMDWYQKKPGSSP | Anti-CD20 Leu16 scFv |
| | KPWIYATSNLASGVPARFSGSGSGTSYSLTISRVEAEDAATYY | entire sequence, with |
| | CQQWSFNPPTFGGGTKLEIKGSTSGSGKPGSGEGSTKGEVQLQ | Whitlow linker |
| | QSGAELVKPGASVKMSCKASGYTFTSYNMHWVKQTPGQGLE | |
| | WIGAIYPGNGDTSYNQKFKGKATLTADKSSSTAYMQLSSLTS | |
| | EDSADYYCARSNYYGSSYWFFDVWGAGTTVTVSS | |
| 415 | DIVLTQSPAILSASPGEKVTMTCRASSSVNYMDWYQKKPGSSP | Anti-CD20 Leu16 scFv |
| | KPWIYATSNLASGVPARFSGSGSGTSYSLTISRVEAEDAATYY | light chain variable |
| | CQQWSFNPPTFGGGTKLEIK | region |
| 416 | RASSSVNYMD | Anti-CD20 Leu16 scFv |
| | | light chain CDR1 |
| 417 | ATSNLAS | Anti-CD20 Leu16 scFv |
| | | light chain CDR2 |
| 418 | QQWSFNPPT | Anti-CD20 Leu16 scFv |
| | | light chain CDR3 |
| 419 | EVQLQQSGAELVKPGASVKMSCKASGYTFTSYNMHWVKQTP | Anti-CD20 Leu16 scFv |
| | GQGLEWIGAIYPGNGDTSYNQKFKGKATLTADKSSSTAYMQL | heavy chain |
| | SSLTSEDSADYYCARSNYYGSSYWFFDVWGAGTTVTVSS | |
| 420 | SYNMH | Anti-CD20 Leu16 scFv |
| | | heavy chain CDR1 |
| 421 | AIYPGNGDTSYNQKFKG | Anti-CD20 Leu16 scFv |
| | | heavy chain CDR2 |
| 422 | SNYYGSSYWFFDV | Anti-CD20 Leu16 scFv |
| | | heavy chain CDR3 |
| 423 | QVQLQQSGPGLVKPSQTLSLTCAISGDSVSSNSAAWNWIRQSP | Anti-CD22 m971 scFv |
| | SRGLEWLGRTYYRSKWYNDYAVSVKSRITINPDTSKNQFSLQ | entire sequence, with |
| | LNSVTPEDTAVYYCAREVTGDLEDAFDIWGQGTMVTVSSGG | 3xG₄S linker |
| | GGSGGGGSGGGGSDIQMTQSPSSLSASVGDRVTITCRASQTIW | |
| | SYLNWYQQRPGKAPNLLIYAASSLQSGVPSRFSGRGSGTDFTL | |
| | TISSLQAEDFATYYCQQSYSIPQTFGQGTKLEIK | |
| 424 | QVQLQQSGPGLVKPSQTLSLTCAISGDSVSSNSAAWNWIRQSP | Anti-CD22 m971 scFv |
| | SRGLEWLGRTYYRSKWYNDYAVSVKSRITINPDTSKNQFSLQ | heavy chain variable |
| | LNSVTPEDTAVYYCAREVTGDLEDAFDIWGQGTMVTVSS | region |
| 425 | GDSVSSNSAA | Anti-CD22 m971 scFv |
| | | heavy chain CDR1 |
| 426 | TYYRSKWYN | Anti-CD22 m971 scFv |
| | | heavy chain CDR2 |
| 427 | AREVTGDLEDAFDI | Anti-CD22 m971 scFv |
| | | heavy chain CDR3 |
| 428 | DIQMTQSPSSLSASVGDRVTITCRASQTIWSYLNWYQQRPGK | Anti-CD22 m971 scFv |
| | APNLLIYAASSLQSGVPSRFSGRGSGTDFTLTISSLQAEDFATY | light chain |
| | YCQQSYSIPQTFGQGTKLEIK | |
| 429 | QTIWSY | Anti-CD22 m971 scFv |
| | | light chain CDR1 |
| 430 | AAS | Anti-CD22 m971 scFv |
| | | light chain CDR2 |
| 431 | QQSYSIPQT | Anti-CD22 m971 scFv |
| | | light chain CDR3 |
| 432 | QVQLQQSGPGMVKPSQTLSLTCAISGDSVSSNSVAWNWIRQS | Anti-CD22 m971-L7 |
| | PSRGLEWLGRTYYRSTWYNDYAVSMKSRITINPDTNKNQFSL | scFv entire sequence, |
| | QLNSVTPEDTAVYYCAREVTGDLEDAFDIWGQGTMVTVSSG | with 3xG4S linker |
| | GGGSGGGGSGGGGSDIQMIQSPSSLSASVGDRVTITCRASQTI | |
| | WSYLNWYRQRPGEAPNLLIYAASSLQSGVPSRFSGRGSGTDF | |
| | TLTISSLQAEDFATYYCQQSYSIPQTFGQGTKLEIK | |
| 433 | QVQLQQSGPGMVKPSQTLSLTCAISGDSVSSNSVAWNWIRQS | Anti-CD22 m971-L7 |
| | PSRGLEWLGRTYYRSTWYNDYAVSMKSRITINPDTNKNQFSL | scFv heavy chain |
| | QLNSVTPEDTAVYYCAREVTGDLEDAFDIWGQGTMVTVSS | variable region |
| 434 | GDSVSSNSVA | Anti-CD22 m971-L7 |
| | | scFv heavy chain |
| | | CDR1 |
| 435 | TYYRSTWYN | Anti-CD22 m971-L7 |
| | | scFv heavy chain |
| | | CDR2 |
| 436 | AREVTGDLEDAFDI | Anti-CD22 m971-L7 |
| | | scFv heavy chain |
| | | CDR3 |
| 437 | DIQMIQSPSSLSASVGDRVTITCRASQTIWSYLNWYRQRPGEA | Anti-CD22 m971-L7 |
| | PNLLIYAASSLQSGVPSRFSGRGSGTDFTLTISSLQAEDFATYY | scFv light chain |
| | CQQSYSIPQTFGQGTKLEIK | variable region |
| 438 | QTIWSY | Anti-CD22 m971-L7 |
| | | scFv light chain CDR1 |
| 439 | AAS | Anti-CD22 m971-L7 |
| | | scFv light chain CDR2 |
| 440 | QQSYSIPQT | Anti-CD22 m971-L7 |
| | | scFv light chain CDR3 |
| 441 | DIVLTQSPASLAMSLGKRATISCRASESVSVIGAHLIHWYQQK | Anti-BCMA C11D5.3 |
| | PGQPPKLLIYLASNLETGVPARFSGSGSGTDFTLTIDPVEEDDV | scFv entire sequence, |
| | AIYSCLQSRIFPRTFGGGTKLEIKGSTSGSGKPGSGEGSTKGQIQ | with Whitlow linker |
| | LVQSGPELKKPGETVKISCKASGYTFTDYSINWVKRAPGKGL | |
| | KWMGWINTETREPAYAYDFRGRFAFSLETSASTAYLQINNLK | |
| | YEDTATYFCALDYSYAMDYWGQGTSVTVSS | |
| 442 | DIVLTQSPASLAMSLGKRATISCRASESVSVIGAHLIHWYQQK | Anti-BCMA C11D5.3 |
| | PGQPPKLLIYLASNLETGVPARFSGSGSGTDFTLTIDPVEEDDV | scFv light chain |
| | AIYSCLQSRIFPRTFGGGTKLEIK | variable region |
| 443 | RASESVSVIGAHLIH | Anti-BCMA C11D5.3 |
| | | scFv light chain CDR1 |
| 444 | LASNLET | Anti-BCMA C11D5.3 |
| | | scFv light chain CDR2 |
| 445 | LQSRIFPRT | Anti-BCMA C11D5.3 |
| | | scFv light chain CDR3 |
| 446 | QIQLVQSGPELKKPGETVKISCKASGYTFTDYSINWVKRAPGK | Anti-BCMA C11D5.3 |
| | GLKWMGWINTETREPAYAYDFRGRFAFSLETSASTAYLQINN | scFv heavy chain |
| | LKYEDTATYFCALDYSYAMDYWGQGTSVTVSS | variable region |
| 447 | DYSIN | Anti-BCMA C11D5.3 |
| | | scFv heavy chain |
| | | CDR1 |
| 448 | WINTETREPAYAYDFRG | Anti-BCMA C11D5.3 |
| | | scFv heavy chain |
| | | CDR2 |
| 449 | DYSYAMDY | Anti-BCMA C11D5.3 |
| | | scFv heavy chain |
| | | CDR3 |
| 450 | DIVLTQSPPSLAMSLGKRATISCRASESVTILGSHLIYWYQQKP | Anti-BCMA C12A3.2 |
| | GQPPTLLIQLASNVQTGVPARFSGSGSRTDFTLTIDPVEEDDVA | scFv entire sequence, |
| | VYYCLQSRTIPRTFGGGTKLEIKGSTSGSGKPGSGEGSTKGQIQ | with Whitlow linker |
| | LVQSGPELKKPGETVKISCKASGYTFRHYSMNWVKQAPGKG | |
| | LKWMGRINTESGVPIYADDFKGRFAFSVETSASTAYLVINNLK | |
| | DEDTASYFCSNDYLYSLDFWGQGTALTVSS | |
| 451 | DIVLTQSPPSLAMSLGKRATISCRASESVTILGSHLIYWYQQKP | Anti-BCMA C12A3.2 |
| | GQPPTLLIQLASNVQTGVPARFSGSGSRTDFTLTIDPVEEDDVA | scFv light chain |
| | VYYCLQSRTIPRTFGGGTKLEIK | variable region |
| 452 | RASESVTILGSHLIY | Anti-BCMA C12A3.2 |
| | | scFv light chain CDR1 |
| 453 | LASNVQT | Anti-BCMA C12A3.2 |
| | | scFv light chain CDR2 |
| 454 | LQSRTIPRT | Anti-BCMA C12A3.2 |
| | | scFv light chain CDR3 |
| 455 | QIQLVQSGPELKKPGETVKISCKASGYTFRHYSMNWVKQAPG | Anti-BCMA C12A3.2 |
| | KGLKWMGRINTESGVPIYADDFKGRFAFSVETSASTAYLVINN | scFv heavy chain |
| | LKDEDTASYFCSNDYLYSLDFWGQGTALTVSS | variable region |
| 456 | HYSMN | Anti-BCMA C12A3.2 |
| | | scFv heavy chain |
| | | CDR1 |
| 457 | RINTESGVPIYADDFKG | Anti-BCMA C12A3.2 |
| | | scFv heavy chain |
| | | CDR2 |
| 458 | DYLYSLDF | Anti-BCMA C12A3.2 |
| | | scFv heavy chain |
| | | CDR3 |
| 459 | EVQLLESGGGLVQPGGSLRLSCAASGFTFSSYAMSWVRQAPG | Anti-BCMA FHVH33 |
| | KGLEWVSSISGSGDYIYYADSVKGRFTISRDISKNTLYLQMNS | entire sequence |
| | LRAEDTAVYYCAKEGTGANSSLADYRGQGTLVTVSS | |
| 460 | GFTFSSYA | Anti-BCMA FHVH33 |
| | | CDR1 |
| 461 | ISGSGDYI | Anti-BCMA FHVH33 |
| | | CDR2 |
| 462 | AKEGTGANSSLADY | Anti-BCMA FHVH33 |
| | | CDR3 |
| 463 | DIQMTQSPSSLSASVGDRVTITCRASQSISSYLNWYQQKPGKA | Anti-BCMA CT103A |
| | PKLLIYAASSLQSGVPSRFSGSGSGTDFTLTISSLQPEDFATYYC | scFv entire sequence, |
| | QQKYDLLTFGGGTKVEIKGSTSGSGKPGSGEGSTKGQLQLQE | with Whitlow linker |
| | SGPGLVKPSETLSLTCTVSGGSISSSSYYWGWIRQPPGKGLEWI | |
| | GSISYSGSTYYNPSLKSRVTISVDTSKNQFSLKLSSVTAADTAV | |
| | YYCARDRGDTILDVWGQGTMVTVSS | |
| 464 | DIQMTQSPSSLSASVGDRVTITCRASQSISSYLNWYQQKPGKA | Anti-BCMA CT103A |
| | PKLLIYAASSLQSGVPSRFSGSGSGTDFTLTISSLQPEDFATYYC | scFv light chain |
| | QQKYDLLTFGGGTKVEIK | variable region |
| 465 | QSISSY | Anti-BCMA CT103A |
| | | scFv light chain CDR1 |
| 466 | AAS | Anti-BCMA CT103A |
| | | scFv light chain CDR2 |
| 467 | QQKYDLLT | Anti-BCMA CT103A |
| | | scFv light chain CDR3 |
| 468 | QLQLQESGPGLVKPSETLSLTCTVSGGSISSSSYYWGWIRQPPG | Anti-BCMA CT103A |
| | KGLEWIGSISYSGSTYYNPSLKSRVTISVDTSKNQFSLKLSSVT | scFv heavy chain |
| | AADTAVYYCARDRGDTILDVWGQGTMVTVSS | variable region |
| 469 | GGSISSSSYY | Anti-BCMA CT103A |
| | | scFv heavy chain |
| | | CDR1 |
| 470 | ISYSGST | Anti-BCMA CT103A |
| | | scFv heavy chain |
| | | CDR2 |
| 471 | ARDRGDTILDV | Anti-BCMA CT103A |
| | | scFv heavy chain |
| | | CDR3 |
| 472 | atggccttaccagtgaccgccttgctcctgccgctggccttgctgctccacgccgccaggccgga | Exemplary BCMA |
| | catccagatgacccagtctccatcctccctgtctgcatctgtaggagacagagtcaccatcacttgc | CAR nucleotide |
| | cgggcaagtcagagcattagcagctatttaaattggtatcagcagaaaccagggaaagcccctaa | sequence |
| | gctcctgatctatgctgcatccagtttgcaaagtggggtcccatcaaggttcagtggcagtggatct | |
| | gggacagatttcactctcaccatcagcagtctgcaacctgaagattttgcaacttactactgtcagca | |
| | aaaatacgacctcctcacttttggcggagggaccaaggttgagatcaaaggcagcaccagcggct | |
| | ccggcaagcctggctctggcgagggcagcacaaagggacagctgcagctgcaggagtcgggc | |
| | ccaggactggtgaagccttcggagaccctgtccctcacctgcactgtctctggtggctccatcagc | |
| | agtagtagttactactggggctggatccgccagcccccagggaaggggctggagtggattggga | |
| | gtatctcctatagtgggagcacctactacaacccgtccctcaagagtcgagtcaccatatccgtaga | |
| | cacgtccaagaaccagttctccctgaagctgagttctgtgaccgccgcagacacggcggtgtact | |
| | actgcgccagagatcgtggagacaccatactagacgtatggggtcagggtacaatggtcaccgtc | |
| | agctcattcgtgcccgtgttcctgcccgccaaacctaccaccacccctgcccctagacctcccacc | |
| | ccagccccaacaatcgccagccagcctctgtctctgcggcccgaagcctgtagacctgctgccg | |
| | gcggagccgtgcacaccagaggcctggacttcgcctgcgacatctacatctgggcccctctggc | |
| | cggcacctgtggcgtgctgctgctgagcctggtgatcaccctgtactgcaaccaccggaacaaac | |
| | ggggcagaaagaaactcctgtatatattcaaacaaccatttatgagaccagtacaaactactcaaga | |
| | ggaagatggctgtagctgccgatttccagaagaagaagaaggaggatgtgaactgagagtgaag | |
| | ttcagcagatccgccgacgcccctgcctaccagcagggacagaaccagctgtacaacgagctga | |
| | acctgggcagacgggaagagtacgacgtgctggacaagcggagaggccgggaccccgagat | |
| | gggcggaaagcccagacggaagaacccccaggaaggcctgtataacgaactgcagaaagaca | |
| | agatggccgaggcctacagcgagatcggcatgaagggcgagcggaggcgcggcaagggcca | |
| | cgatggcctgtaccagggcctgagcaccgccaccaaggacacctacgacgccctgcacatgca | |
| | ggccctgccccccaga | |
| 473 | MALPVTALLLPLALLLHAARPDIQMTQSPSSLSASVGDRVTIT | Exemplary BCMA |
| | CRASQSISSYLNWYQQKPGKAPKLLIYAASSLQSGVPSRFSGS | CAR amino acid |
| | GSGTDFTLTISSLQPEDFATYYCQQKYDLLTFGGGTKVEIKGS | sequence |
| | TSGSGKPGSGEGSTKGQLQLQESGPGLVKPSETLSLTCTVSGG | |
| | SISSSSYYWGWIRQPPGKGLEWIGSISYSGSTYYNPSLKSRVTIS | |
| | VDTSKNQFSLKLSSVTAADTAVYYCARDRGDTILDVWGQGT | |
| | MVTVSSFVPVFLPAKPTTTPAPRPPTPAPTIASQPLSLRPEACRP | |
| | AAGGAVHTRGLDFACDIYIWAPLAGTCGVLLLSLVITLYCNH | |
| | RNKRGRKKLLYIFKQPFMRPVQTTQEEDGCSCRFPEEEEGGCE | |
| | LRVKFSRSADAPAYQQGQNQLYNELNLGRREEYDVLDKRRG | |
| | RDPEMGGKPRRKNPQEGLYNELQKDKMAEAYSEIGMKGERR | |
| | RGKGHDGLYQGLSTATKDTYDALHMQALPPR | |
| 474 | RYTMH | CD3 CDR-H1 |
| 475 | YINPSRGYTNYNQKFKD | CD3 CDR-H2 |
| 476 | YYDDHYCLDY | CD3 CDR-H3 |
| 477 | RASSSVSYMN | CD3 CDR-L1 |
| 478 | DTSKVAS | CD3 CDR-L2 |
| 479 | QQWSSNPLT | CD3 CDR-L3 |
| 480 | DIKLQQSGAELARPGASVKMSCKTSGYTFTRYTMHWVKQRP | CD3 VH |
| | GQGLEWIGYINPSRGYTNYNQKFKDKATLTTDKSSSTAYMQL | |
| | SSLTSEDSAVYYCARYYDDHYCLDYWGQGTTLTVSSVE | |
| 481 | DIQLTQSPAIMSASPGEKVTMTCRASSSVSYMNWYQQKSGTS | CD3 VL |
| | PKRWIYDTSKVASGVPYRFSGSGSGTSYSLTISSMEAEDAATY | |
| | YCQQWSSNPLTFGAGTKLELK | |
| 482 | DIKLQQSGAELARPGASVKMSCKTSGYTFTRYTMHWVKQRP | CD3 scFv |
| | GQGLEWIGYINPSRGYTNYNQKFKDKATLTTDKSSSTAYMQL | |
| | SSLTSEDSAVYYCARYYDDHYCLDYWGQGTTLTVSSVEGGS | |
| | GGSGGSGGSGGVDDIQLTQSPAIMSASPGEKVTMTCRASSSVS | |
| | YMNWYQQKSGTSPKRWIYDTSKVASGVPYRFSGSGSGTSYSL | |
| | TISSMEAEDAATYYCQQWSSNPLTFGAGTKLELK | |
| 483 | SYAIS | CDR-H1 |
| 484 | IIDPSDGNTNYAQNFQG | CDR-H2 |
| 485 | ERAAAGYYYYMDV | CDR-H3 |
| 486 | RASQSISSYLN | CDR-L1 |
| 487 | AASSLQS | CDR-L2 |
| 488 | QQSYSTPLT | CDR-L3 |
| 489 | QVQLVQSGAEVKKPGASVKVSCKASGGTFSSYAISWVRQAPG | CD8 scFv |
| | QGLEWMGIIDPSDGNTNYAQNFQGRVTMTRDTSTSTVYMELS | |
| | SLRSEDTAVYYCAKERAAAGYYYYMDVWGQGTTVTVSSGG | |
| | GGSGGGGSGGGGSDIQMTQSPSSLSASVGDRVTITCRASQSISS | |
| | YLNWYQQKPGKAPKLLIYAASSLQSGVPSRFSGSGSGTDFTLT | |
| | ISSLQPEDFATYYCQQSYSTPLTFGGGTKVEIKR | |
| 490 | DYYIQ | CDR-H1 |
| 491 | WINPNSGGTSYAQKFQG | CDR-H2 |
| 492 | EGDYYYGMDA | CDR-H3 |
| 493 | RSSQSLLHSNGYNYLD | CDR-L1 |
| 494 | LGSNRAS | CDR-L2 |
| 495 | MQGLQTPHT | CDR-L3 |
| 496 | QVQLVQSGAEVKKPGASVKVSCKASGYTFTDYYIQWVRQAP | CD8 scFv |
| | GQGLEWMGWINPNSGGTSYAQKFQGRVTMTRDTSTSTVYME | |
| | LSSLRSEDTAVYYCAKEGDYYYGMDAWGQGTMVTVSSGGG | |
| | GSGGGGSGGGGSDIVMTQSPLSLPVTPGEPASISCRSSQSLLHS | |
| | NGYNYLDWYLQKPGQSPQLLIYLGSNRASGVPDRFSGSGSGT | |
| | DFTLKISRVEAEDVGVYYCMQGLQTPHTFGQGTKVEIKR | |
| 497 | SYYMH | CDR-H1 |
| 498 | GFDPEDGETIYAQKFQG | CDR-H2 |
| 499 | DQGWGMDV | CDR-H3 |
| 500 | QQTYSTPYT | CDR-L3 |
| 501 | QVQLVQSGAEVKKPGASVKVSCKASGYTFTSYYMHWVRQA | CD8 scFv |
| | PGQGLEWMGGFDPEDGETIYAQKFQGRVTMTRDTSTSTVYM | |
| | ELSSLRSEDTAVYYCARDQGWGMDVWGQGTTVTVSSGGGG | |
| | SGGGGSGGGGSDIQMTQSPSSLSASVGDRVTITCRASQSISSYL | |
| | NWYQQKPGKAPKLLIYAASSLQSGVPSRFSGSGSGTDFTLTISS | |
| | LQPEDFATYYCQQTYSTPYTFGQGTKLEIKR | |
| 502 | NHYMH | CDR-H1 |
| 503 | WMNPNSGNTGYAQKFQG | CDR-H2 |
| 504 | SESGSDLDY | CDR-H3 |
| 505 | RASQTIGNYVN | CDR-L1 |
| 506 | GASNLHT | CDR-L2 |
| 507 | QQTYSAPLT | CDR-L3 |
| 508 | QVQLVQSGAEVKKPGASVKVSCKASGYTFTNHYMHWVRQA | CD8 scFv |
| | PGQGLEWMGWMNPNSGNTGYAQKFQGRVTMTRDTSTSTVY | |
| | MELSSLRSEDTAVYYCASSESGSDLDYWGQGTLVTVSSGGGG | |
| | SGGGGSGGGGSDIQMTQSPSSLSASVGDRVTITCRASQTIGNY | |
| | VNWYQQKPGKAPKLLIYGASNLHTGVPSRFSGSGSGTDFTLTI | |
| | SSLQPEDFATYYCQQTYSAPLTFGGGTKVEIKR | |
| 509 | GYVMG | CDR-H1 |
| 510 | AISRGGLSTSYADSVKG | CDR-H2 |
| 511 | DRSDLYEITAASNIDS | CDR-H3 |
| 512 | QVQLVESGGGLVQAGGSLRLSCAASGRTNSNYAMGWFRQAP | CD117 VHH (b12) |
| | GKEREFVSVIGRGLGSTYYADSVKGRFTISRDNAKNTVYLQM | |
| | NSLKPEDAAVYYCAADLEFVFDEMGWIPGEHAYWGRGTLVT | |
| | VSS | |
| 513 | QVQLVESGGRLVQAGESLRLSCSASGGTFSSYLMAWFRQVPG | CD117 VHH (b34) |
| | KEREIVAFITWHGHTTYYTDSVKGRFAISRDNARNTMYLQMN | |
| | SLRPEDTAVYYCAADQRQEFSGTYSREYNYWGQGTQVTVSS | |
| 514 | QVQLVESGGGLVQPGGSLRLSCAASGQTFTDYHHHVAWFRQ | CD117 VHH (b41) |
| | APGRERDFVAVISWSTGNTDYADSQRARFTISKDNAKNTVYL | |
| | QMNNLKFEDTAVYYCAARDYHPRFSYSADDYNYWGQGTLV | |
| | TVSS | |
| 515 | EVQLVESGGGLVQAGGSLRLSCAASGSSINEMGYYRQAPGKE | CD117 VHH (b46) |
| | RAPRELVATITSGGSTNYADSVKGRFTISRDNAKNTVYLQMN | |
| | SLKPEDTAVYYCAALRIMTMGRPYDYWGQGTQVTVSS | |
| 516 | EIQLQQSGPDLMKPGASVKISCKASGYSFTNYYVHWVKQSLD | CD133 scFv (b3) |
| | KSLEWIGYVDPFNGDFNYNQKFKDKATLTVDKSSSTAYMHLS | |
| | SLTSEDSAVYYCARGGLDWYDTSYWYFDVWGAGTAVTVSS | |
| | GGGGSGGGGSGGGGSNIVMTQSPKSMSMSLGERVTLSCKASE | |
| | NVDTYVSWYQQKPEQSPKVLIYGASNRYTGVPDRFTGSGSAT | |
| | DFSLTISNVQAEDLADYHCGQSYRYPLTFGAGTKLELK | |
| 517 | EIQLQQSGPDLMKPGASVKISCKASGYSFTNYYVHWVKQSLD | CD133 VH (b3) |
| | KSLEWIGYVDPFNGDFNYNQKFKDKATLTVDKSSSTAYMHLS | |
| | SLTSEDSAVYYCARGGLDWYDTSYWYFDVWGAGTAVTVSS | |
| 518 | NYYVH | CD133 CDR-H1 (b3) |
| | | (Kabat) |
| 519 | YVDPFNGDFNYNQKFKD | CD133 CDR-H2 (b3) |
| | | (Kabat) |
| 520 | GGLDWYDTSYWYFDV | CD133 CDR-H3 (b3) |
| | | (Kabat, Chothia) |
| 521 | NIVMTQSPKSMSMSLGERVTLSCKASENVDTYVSWYQQKPE | CD133 VL (b3) |
| | QSPKVLIYGASNRYTGVPDRFTGSGSATDFSLTISNVQAEDLA | |
| | DYHCGQSYRYPLTFGAGTKLELK | |
| 522 | KASENVDTYVS | CD133 CDR-L1 (b3) |
| | | (Kabat, Chothia) |
| 523 | GASNRYT | CD133 CDR-L2 (b3) |
| | | (Kabat) |
| 524 | GQSYRYPLT | CD133 CDR-L3 (b3) |
| | | (Kabat) |
| 525 | EVQLVESGGGLVQPGGSLRLSCAASGFNIYYYGSMHWVRQA | CD133 scFv (b4) |
| | PGKGLEWVASISPYYGSTYYADSVKGRFTISADTSKNTAYLQ | |
| | MNSLRAEDTAVYYCARHASSGYGHYAVYGIDYWGQGTLVT | |
| | VSSGGGGSGGGGSGGGGSDIQMTQSPSSLSASVGDRVTITCRA | |
| | SQGSSYVAWYQQKPGKAPKLLIYSASYLYSGVPSRESGSRSGT | |
| | DFTLTISSLQPEDFATYYCQQGVWSLITFGQGTKVEIK | |
| 526 | EVQLVESGGGLVQPGGSLRLSCAASGFNIYYYGSMHWVRQA | CD133 VH (b4) |
| | PGKGLEWVASISPYYGSTYYADSVKGRFTISADTSKNTAYLQ | |
| | MNSLRAEDTAVYYCARHASSGYGHYAVYGIDYWGQGTLVT | |
| | VSS | |
| 527 | YYGSMH | CD133 CDR-H1 (b4) |
| | | (Kabat) |
| 528 | SISPYYGSTYYADSVKG | CD133 CDR-H2 (b4) |
| | | (Kabat) |
| 529 | HASSGYGHYAVYGIDY | CD133 CDR-H3 (b4) |
| | | (Kabat, Chothia) |
| 530 | DIQMTQSPSSLSASVGDRVTITCRASQGSSYVAWYQQKPGKA | CD133 VL (b4) |
| | PKLLIYSASYLYSGVPSRFSGSRSGTDFTLTISSLQPEDFATYYC | |
| | QQGVWSLITFGQGTKVEIK | |
| 531 | RASQGSSYVA | CD133 CDR-L1 (b4) |
| | | (Kabat, Chothia) |
| 532 | SASYLYS | CD133 CDR-L1 (b4) |
| | | (Kabat, Chothia) |
| 533 | QQGVWSLIT | CD133 CDR-L1 (b4) |
| | | (Kabat, Chothia) |
| 534 | EVKLVESGPELVKPGETVKISCKASGYTFTDYSMHWVRQAPG | CD133 scFv (b1) |
| | KGLEWMGWINTETGEPSYADDFKGRFAFSLDTSASTAYLQIN | |
| | SLRSEDTATYFCATDYGDYFDYWGQGTTVTVSSGGGGSGGG | |
| | GSGGGGSDIVLSQSPAIMSASPGEKVTISCSASSSVSYMYWYQ | |
| | QKPGSSPKPWIYRTSNLASGVPARFSGSGSGTSYSLTISSMEPE | |
| | DAATYYCQQYHSYPPTFGAGTKLELK | |
| 535 | EVKLVESGPELVKPGETVKISCKASGYTFTDYSMHWVRQAPG | CD133 VH (b1) |
| | KGLEWMGWINTETGEPSYADDFKGRFAFSLDTSASTAYLQIN | |
| | SLRSEDTATYFCATDYGDYFDYWGQGTTVTVSS | |
| 536 | DYSMH | CD133 CDR-H1 (b1) |
| | | (Kabat) |
| 537 | WINTETGEPSYADDFKG | CD133 CDR-H2 (b1) |
| | | (Kabat) |
| 538 | DYGDYFDY | CD133 CDR-H3 (b1) |
| | | (Kabat, Chothia) |
| 539 | DIVLSQSPAIMSASPGEKVTISCSASSSVSYMYWYQQKPGSSP | CD133 VL (b1) |
| | KPWIYRTSNLASGVPARFSGSGSGTSYSLTISSMEPEDAATYY | |
| | CQQYHSYPPTFGAGTKLELK | |
| 540 | SASSSVSYMY | CD133 CDR-L1 (b1) |
| | | (Kabat, Chothia) |
| 541 | RTSNLAS | CD133 CDR-L2 (b1) |
| | | (Kabat, Chothia) |
| 542 | QQYHSYPPT | CD133 CDR-L3 (b1) |
| | | (Kabat, Chothia) |
| 543 | DVQLQESGPGLVKPSQSLSLTCSVTGYSITSGYYWSWIRQFPG | CD133 scFv (b2) |
| | NKLEWMGYISYDGSNNYNPSLKNRISITRDTSKNQIFLKLNSV | |
| | TTEDTATYYCTRVYFGHPYYYGMDYWGQGISVTVGGGGSGG | |
| | GGSGGGGSQIVLTQSPAIMSASLGERVTMTCTASSSVSSFYLH | |
| | WYRQKPGSSPKLWIYSTSNLASGVPARFSGSGSGTSYSLTISS | |
| | MEAEDAATYYCHQYHRSPYMYMFGGGTKLEIK | |
| 544 | DVQLQESGPGLVKPSQSLSLTCSVTGYSITSGYYWSWIRQFPG | CD133 VH (b2) |
| | NKLEWMGYISYDGSNNYNPSLKNRISITRDTSKNQIFLKLNSV | |
| | TTEDTATYYCTRVYFGHPYYYGMDYWGQGISVTV | |
| 545 | SGYYWS | CD133 CDR-H1 (b2) |
| | | (Kabat) |
| 546 | YISYDGSNNYNPSLKN | CD133 CDR-H2 (b2) |
| | | (Kabat) |
| 547 | VYFGHPYYYGMDY | CD133 CDR-H3 (b2) |
| | | (Kabat, Chothia) |
| 548 | QIVLTQSPAIMSASLGERVTMTCTASSSVSSFYLHWYRQKPGS | CD133 VL (b2) |
| | SPKLWIYSTSNLASGVPARFSGSGSGTSYSLTISSMEAEDAATY | |
| | YCHQYHRSPYMYMFGGGTKLEIK | |
| 549 | TASSSVSSFYLH | CD133 CDR-L1 (b2) |
| | | (Kabat, Chothia) |
| 550 | STSNLAS | CD133 CDR-L2 (b2) |
| | | (Kabat, Chothia) |
| 551 | HQYHRSPYMYM | CD133 CDR-L3 (b2) |
| | | (Kabat, Chothia) |
| 552 | EVQLVESGGGLVQPGGSLRLSCAASGFNLSSSSIHWVRQAPG | CD133 scFv (b5) |
| | KGLEWVAYIYPYYSYTYYADSVKGRFTISADTSKNTAYLQM | |
| | NSLRAEDTAVYYCARFGSVAGFDYWGQGTLVTVSSGGGGSG | |
| | GGGSGGGGSDIQMTQSPSSLSASVGDRVTITCRASQSVSSAVA | |
| | WYQQKPGKAPKLLIYSASSLYSGVPSRFSGSRSGTDFTLTISSL | |
| | QPEDFATYYCQQYSHAGHLFTFGQGTKVEIK | |
| 553 | EVQLVESGGGLVQPGGSLRLSCAASGFNLSSSSIHWVRQAPG | CD133 VH (b5) |
| | KGLEWVAYIYPYYSYTYYADSVKGRFTISADTSKNTAYLQM | |
| | NSLRAEDTAVYYCARFGSVAGFDYWGQGTLVTVSS | |
| 554 | SSSIH | CD133 CDR-H1 (b5) |
| | | (Kabat) |
| 555 | YIYPYYSYTYYADSVKG | CD133 CDR-H2 (b5) |
| | | (Kabat) |
| 556 | FGSVAGFDY | CD133 CDR-H3 (b5) |
| | | (Kabat, Chothia) |
| 557 | DIQMTQSPSSLSASVGDRVTITCRASQSVSSAVAWYQQKPGK | CD133 VL (b5) |
| | APKLLIYSASSLYSGVPSRFSGSRSGTDFTLTISSLQPEDFATYY | |
| | CQQYSHAGHLFTFGQGTKVEIK | |
| 558 | RASQSVSSAVA | CD133 CDR-L1 (b5) |
| | | (Kabat, Chothia) |
| 559 | SASSLYS | CD133 CDR-L2 (b5) |
| | | (Kabat, Chothia) |
| 560 | QQYSHAGHLFT | CD133 CDR-L3 (b5) |
| | | (Kabat, Chothia) |
| 561 | MMADSKLVSLNNNLSGKIKDQGKVIKNYYGTMDIKKINDGL | Henipavirus protein G |
| | LDSKILGAFNTVIALLGSIIIIVMNIMIIQNYTRTTDNQALIKESL | (GENBANK ID: |
| | QSVQQQIKALTDKIGTEIGPKVSLIDTSSTITIPANIGLLGSKISQ | AF017149) |
| | STSSINENVNDKCKFTLPPLKIHECNISCPNPLPFREYRPISQGV | |
| | SDLVGLPNQICLQKTTSTILKPRLISYTLPINTREGVCITDPLLA | |
| | VDNGFFAYSHLEKIGSCTRGIAKQRIIGVGEVLDRGDKVPSMF | |
| | MTNVWTPPNPSTIHHCSSTYHEDFYYTLCAVSHVGDPILNSTS | |
| | WTESLSLIRLAVRPKSDSGDYNQKYIAITKVERGKYDKVMPY | |
| | GPSGIKQGDTLYFPAVGFLPRTEFQYNDSNCPIIHCKYSKAENC | |
| | RLSMGVNSKSHYILRSGLLKYNLSLGGDIILQFIEIADNRLTIGS | |
| | PSKIYNSLGQPVFYQASYSWDTMIKLGDVDTVDPLRVQWRN | |
| | NSVISRPGQSQCPRFNVCPEVCWEGTYNDAFLIDRLNWVSAG | |
| | VYLNSNQTAENPVFAVFKDNEILYQVPLAEDDTNAQKTITDC | |
| | FLLENVIWCISLVEIYDTGDSVIRPKLFAVKIPAQCSES | |
| 562 | MLSQLQKNYLDNSNQQGDKMNNPDKKLSVNFNPLELDKGQ | Henipavirus protein G |
| | KDLNKSYYVKNKNYNVSNLLNESLHDIKFCIYCIFSLLIIITIINII | (GENBANK ID: |
| | TISIVITRLKVHEENNGMESPNLQSIQDSLSSLTNMINTEITPRIG | JQ001776) |
| | ILVTATSVTLSSSINYVGTKTNQLVNELKDYITKSCGFKVPELK | |
| | LHECNISCADPKISKSAMYSTNAYAELAGPPKIFCKSVSKDPDF | |
| | RLKQIDYVIPVQQDRSICMNNPLLDISDGFFTYIHYEGINSCKK | |
| | SDSFKVLLSHGEIVDRGDYRPSLYLLSSHYHPYSMQVINCVPV | |
| | TCNQSSFVFCHISNNTKTLDNSDYSSDEYYITYFNGIDRPKTKK | |
| | IPINNMTADNRYIHFTFSGGGGVCLGEEFIIPVTTVINTDVFTHD | |
| | YCESFNCSVQTGKSLKEICSESLRSPTNSSRYNLNGIMIISQNN | |
| | MTDFKIQLNGITYNKLSFGSPGRLSKTLGQVLYYQSSMSWDT | |
| | YLKAGFVEKWKPFTPNWMNNTVISRPNQGNCPRYHKCPEICY | |
| | GGTYNDIAPLDLGKDMYVSVILDSDQLAENPEITVENSTTILY | |
| | KERVSKDELNTRSTTTSCFLFLDEPWCISVLETNRFNGKSIRPEI | |
| | YSYKIPKYC | |
| 563 | MPQKTVEFINMNSPLERGVSTLSDKKTLNQSKITKQGYFGLGS | Henipavirus protein G |
| | HSERNWKKQKNQNDHYMTVSTMILEILVVLGIMENLIVLTMV | (GENBANK ID: |
| | YYQNDNINQRMAELTSNITVLNLNLNQLTNKIQREIIPRITLIDT | NC_025256) |
| | ATTITIPSAITYILATLTTRISELLPSINQKCEFKTPTLVLNDCRIN | |
| | CTPPLNPSDGVKMSSLATNLVAHGPSPCRNFSSVPTIYYYRIPG | |
| | LYNRTALDERCILNPRLTISSTKFAYVHSEYDKNCTRGFKYYE | |
| | LMTFGEILEGPEKEPRMFSRSFYSPTNAVNYHSCTPIVTVNEG | |
| | YFLCLECTSSDPLYKANLSNSTFHLVILRHNKDEKIVSMPSFNL | |
| | STDQEYVQIIPAEGGGTAESGNLYFPCIGRLLHKRVTHPLCKK | |
| | SNCSRTDDESCLKSYYNQGSPQHQVVNCLIRIRNAQRDNPTW | |
| | DVITVDLTNTYPGSRSRIFGSFSKPMLYQSSVSWHTLLQVAEIT | |
| | DLDKYQLDWLDTPYISRPGGSECPFGNYCPTVCWEGTYNDV | |
| | YSLTPNNDLFVTVYLKSEQVAENPYFAIFSRDQILKEFPLDAWI | |
| | SSARTTTISCFMFNNEIWCIAALEITRLNDDIIRPIYYSFWLPTD | |
| | CRTPYPHTGKMTRVPLRSTYNY | |
| 564 | MATNRDNTITSAEVSQEDKVKKYYGVETAEKVADSISGNKVF | Henipavirus protein G |
| | ILMNTLLILTGAIITITLNITNLTAAKSQQNMLKIIQDDVNAKLE | (GENBANK ID: |
| | MFVNLDQLVKGEIKPKVSLINTAVSVSIPGQISNLQTKFLQKY | NC_025352) |
| | VYLEESITKQCTCNPLSGIFPTSGPTYPPTDKPDDDTTDDDKVD | |
| | TTIKPIEYPKPDGCNRTGDHFTMEPGANFYTVPNLGPASSNSD | |
| | ECYTNPSFSIGSSIYMFSQEIRKTDCTAGEILSIQIVLGRIVDKGQ | |
| | QGPQASPLLVWAVPNPKIINSCAVAAGDEMGWVLCSVTLTA | |
| | ASGEPIPHMFDGFWLYKLEPDTEVVSYRITGYAYLLDKQYDS | |
| | VFIGKGGGIQKGNDLYFQMYGLSRNRQSFKALCEHGSCLGTG | |
| | GGGYQVLCDRAVMSFGSEESLITNAYLKVNDLASGKPVIIGQT | |
| | FPPSDSYKGSNGRMYTIGDKYGLYLAPSSWNRYLRFGITPDIS | |
| | VRSTTWLKSQDPIMKILSTCTNTDRDMCPEICNTRGYQDIFPLS | |
| | EDSEYYTYIGITPNNGGTKNFVAVRDSDGHIASIDILQNYYSIT | |
| | SATISCFMYKDEIWCIAITEGKKQKDNPQRIYAHSYKIRQMCY | |
| | NMKSATVTVGNAKNITIRRY | |
| 565 | NTETGE | CD133 CDR-H2 (b1) |
| | | (Chothia) |
| 566 | GYSITSGY | CD133 CDR-H1 (b2) |
| | | (Chothia) |
| 567 | SYDGS | CD133 CDR-H2 (b2) |
| | | (Chothia) |
| 568 | GYSFTNY | CD133 CDR-H1 (b3) |
| | | (Chothia) |
| 569 | DPFNGD | CD133 CDR-H2 (b3) |
| | | (Chothia) |
| 570 | GFNIYYYG | CD133 CDR-H1 (b4) |
| | | (Chothia) |
| 571 | SPYYGS | CD133 CDR-H2 (b4) |
| | | (Chothia) |
| 572 | GFNLSSS | CD133 CDR-H1 (b5) |
| | | (Chothia) |
| 573 | YPYYSY | CD133 CDR-H2 (b5) |
| | | (Chothia) |
| 574 | MKKINEGLLDSKILSAFNTVIALLGSIVIIVMNIMIIQNYTRSTD | Blinded NiVG protein |
| | NQAVIKDALQGIQQQIKGLADKIGTEIGPKVSLIDTSSTITIPANI | attachment |
| | GLLGSKISQSTASINENVNEKCKFTLPPLKIHECNISCPNPLPFR | glycoprotein + linker + |
| | EYRPQTEGVSNLVGLPNNICLQKTSNQILKPKLISYTLPVVGQS | CD133 scFv (b1) |
| | GTCITDPLLAMDEGYFAYSHLERIGSCSRGVSKQRIIGVGEVL | |
| | DRGDEVPSLFMTNVWTPPNPNTVYHCSAVYNNEFYYVLCAV | |
| | STVGDPILNSTYWSGSLMMTRLAVKPKSNGGGYNQHQLALR | |
| | SIEKGRYDKVMPYGPSGIKQGDTLYFPAVGFLVRTEFKYNDS | |
| | NCPITKCQYSKPENCRLSMGIRPNSHYILRSGLLKYNLSDGENP | |
| | KVVFIEISDQRLSIGSPSKIYDSLGQPVFYQASFSWDTMIKFGD | |
| | VLTVNPLVVNWRNNTVISRPGQSQCPRFNTCPAICAEGVYND | |
| | AFLIDRINWISAGVFLDSNATAANPVFTVFKDNEILYRAQLAS | |
| | EDTNAQKTITNCFLLKNKIWCISLVEIYDTGDNVIRPKLFAVKI | |
| | PEQCTGGGGSGGGGSGGGGSEVKLVESGPELVKPGETVKISC | |
| | KASGYTFTDYSMHWVRQAPGKGLEWMGWINTETGEPSYAD | |
| | DFKGRFAFSLDTSASTAYLQINSLRSEDTATYFCATDYGDYFD | |
| | YWGQGTTVTVSSGGGGSGGGGSGGGGSDIVLSQSPAIMSASP | |
| | GEKVTISCSASSSVSYMYWYQQKPGSSPKPWIYRTSNLASGVP | |
| | ARFSGSGSGTSYSLTISSMEPEDAATYYCQQYHSYPPTFGAGT | |
| | KLELK | |
| 575 | EVQLVESGGGLVQAGGSLRLSCKASGRTNSNYAMGWFRQAP | CD117 VHH (b1) |
| | GKEREFVTVIGRSIGMTYYADSVKGRFTISRDNAKNTVYLQM | |
| | NSLKPEDTAVYYCAADLEFVFGETGWIPGDDAYWGQGTQVT | |
| | VSS | |
| 576 | QVQLVESGGGLVQAGGSLRLSCAASGRTNSNYAMGWFRQAP | CD117 VHH (b2) |
| | GKEREFVSVIGRGLGSTYYADSVKGRFTISRDNAKNTVYLQV | |
| | NSLKPEDTAVYYCAADLEFVFDEMGWIPGEHAYWGRGTLVT | |
| | VSS | |
| 577 | EVQLVESGGGLVQPGGSLRLSCTASGRTNSNYAMGWFRQAP | CD117 VHH (b3) |
| | GKEREFVSVIGRGLGSTYYADSVKGRFTISRDNAKNTVYLQM | |
| | NSLKPEDTAVYYCAADLEFVFDETSWIPGEHAYWGRGTLVTV | |
| | SS | |
| 578 | EVQLVESGGGLVQAGGSLRLSCAASGRTNSNYAMGWFRQAP | CD117 VHH (b4) |
| | GKEREFVTVIGRSIGMTYYADSVKGRFTISRDNAKNTVYLQM | |
| | NSLKPEDTAVYYCAADLEFVFDETGWIPGDDAYWGQGTLVT | |
| | VSS | |
| 579 | QVQLVESGGGLVQAGGSLRLSCAASGRTNSNYAMGWFRQAP | CD117 VHH (b5) |
| | GKEREFVSVIGRGLGSTYYADSVKGRFTISRDNAKNTVYLQM | |
| | NSLKPEDTAVYYCAADLEFVFDGMGWIPGEHAYWGRGTLVT | |
| | VSS | |
| 580 | EVQLVESGGGLVQAGGSLRLSCAASGRTNSNYAMGWFRQAP | CD117 VHH (b6) |
| | GKEREFVSVIGRGLGSTYYADSVKGRFTISRDNAKNTVYLQM | |
| | NSLKPEDTAVYYCAADLEFVEDEMGWIPGEHAYWGRGTLVT | |
| | VSS | |
| 581 | EVQLVESGGRLVRAGGSLRLSCAASGRINSNYAMGWFRQAP | CD117 VHH (b7) |
| | GKEREFVSVIGRGLGSTYYADSVKGRFTISRDNAKNTVYLQM | |
| | NSLKPEDTAVYFCAADLEFVFDEMGWIPGEHAYWGRGTLVT | |
| | VSS | |
| 582 | EVQLVESGGGLVQAGGSLRLSCAASGRTNSNYAMGWFRQAP | CD117 VHH (b8) |
| | GKEREFVSVIGRGLGSTYYADSVKGRFTISRDNAKNTVYLQM | |
| | NSLKPEDTSVYYCAADLEFVFDEMGWIPGEHAYWGRGTQVT | |
| | VSS | |
| 583 | QVQLVESGGGLVQAGGSLGLSCAASGRTFSRMMMGWFRQA | CD117 VHH (b9) |
| | PGKEREFVSVIGRGLGSTYYADSVKGRFTISRDNAKNTVYLQ | |
| | MNSLKPEDTAVYYCAADLEFVFDEMGWIPGEHAYWGRGTQ | |
| | VTVSS | |
| 584 | QVQLVESGGGLVQAGGSLRLSCAASGRINSNYAMGWFRQAP | CD117 VHH (b10) |
| | GKEREFVSVIGRGLGSTYYADSVKGRFTISRDNAKNTVYLQM | |
| | NSLKPEDTAVYYCAADLEFVFDEMGWIPGEHAYWGRGTQVT | |
| | VSS | |
| 585 | QVQLVESGGGLVQAGGSLRLSCAASGRTNSNYAMGWFRQAP | CD117 VHH (b11) |
| | GKEREFVSVIGRGLGSTYCADSVKGRFTTSRDNAKNTVYLQM | |
| | NSLKPEDTAVYYCAADLEFVFDEMGWIPGEHAYWGRGTQVT | |
| | VSS | |
| 586 | EVQLVESGGGLVQAGGSLRLSCAASGRTNSNYAMGWFRQAP | CD117 VHH (b13) |
| | GKEREFVSVIGRGLGSTYYADSVKGRFTISRDNAKNTVYLQM | |
| | NSLKPEDTAVYYCAADLEFVFDEMGWIPGEHAYWGRGTQVT | |
| | VSS | |
| 587 | EVQLVESGGGLVQAGGSLRLSCAASGRINSDYAMGWFRQAP | CD117 VHH (b14) |
| | GKEREFVSVIGRGLGSTYYADSVKGRFTISRDNAKNTVYLQM | |
| | NSLKPEDTAVYYCAADLEFVFDEMGWIPGEHAYWGRGTQVT | |
| | VSS | |
| 588 | EVQLVESGGGLVQAGGSLRLSCAASGRTNSNYAMGWFRQAP | CD117 VHH (b15) |
| | GKEREFVSVIGRGLGSTYYADSVKGRFTISRDNAKNTVYLQM | |
| | NSLKPEDTAVYYCVADLEFVFDEMGWIPGEHAYWGRGTLVT | |
| | VSS | |
| 589 | EVQLVESGGGLAQAGDSLRLSCAASGREDTSYAMGWFRQAP | CD117 VHH (b16) |
| | GKEREFVAGILSGTVKAYEDSVRGRFSISRDKAKNTVYLQMN | |
| | SLKSEDTAVYYCVADVSFGDLINGRSDYWGQGTQVTVSS | |
| 590 | EVQLVESGGGLAQAGDSLRLSCAASGRFDTSYAMGWFRQAP | CD117 VHH (b17) |
| | GKEREFVAGILSGTVKAYEDSVRGRFSISRDKAKNTVYLQMN | |
| | SLKPEDTAVYYCVADVSFGDLTNGRSDYWGQGTQVTVSS | |
| 591 | EVQLVESGGGLAQAGDSLRLSCAASGRFDTSYAMGWFRQAP | CD117 VHH (b18) |
| | GKEREFVAGILSGTVKAYEDSVRGRFSISRDKAKNTVYLQMN | |
| | SLKPEDTAVYYCVADVSFGDLTNGRSDYWGQGTLVTVSS | |
| 592 | QVQLVESGGGLAQAGDSLRLSCAASGRFDTSYAMGWFRQAP | CD117 VHH (b19) |
| | GKEREFVAGILSGTVKAYEDSVRGRFSISRDKAKNTVYLQMN | |
| | SLKSEDTAVYYCVADVSFGDLINGRSDYWGQGTQVTVSS | |
| 593 | EVQLVESGGGLAQAGDSLRLSCAASGRFDTSYAMGWFRQAP | CD117 VHH (b20) |
| | GKEREFVAGILSGTVKAYEDSVRGRFSISRDKAKNTVYLQMN | |
| | SLKSEDTAVYYCVADVSFGDLTNGRSDYWGQGTLVTVSS | |
| 594 | EVQLVESGGGLAQAGDSLRLSCAASGREDTSYAMGWFRQAP | CD117 VHH (b21) |
| | GKEREFVAGILSGAVKAYEDSVRGRFSISRDKAKNTVYLQMN | |
| | SLKPEDTAVYYCVADVSFGDLTNGRSDYWGQGTQVTVSS | |
| 595 | QVQLVESGGGLAQAGDSLRLSCAASGRFDTSYAMGWFRQAP | CD117 VHH (b22) |
| | GKEREFVAGILSGTVKAYEDSVRGRFSISRDKAKNTVYLQMN | |
| | SLKPEDTAVYYCVADVSFGDLINGRSDYWGQGTQVTVSS | |
| 596 | EVQLVESGGGLAQAGDSLRLSCAASGRFDASYAMGWFRQAP | CD117 VHH (b23) |
| | GKEREFVAGILSGTVKAYEDSVRGRFSISRDKAKNTVYLQMN | |
| | SLKSEDTAVYYCVADVSFGDLTNGRSDYWGQGTLVTVSS | |
| 597 | EVQLVESGGGLAQAGDSLRLSCAASGREDTSYAMGWFRQAP | CD117 VHH (b24) |
| | GKEREFVAGILSGTVKAYEDSVRGRFSISRDQAKNTVYLQMN | |
| | SLKPEDTAVYYCVADVSFGDLINGRSDYWGQGTQVTVSS | |
| 598 | EVQLVESGGGLAQAGDSLRLSCAASGRFDTSYAMGWFRQVP | CD117 VHH (b25) |
| | GKERGFVAGILSGTVKAYEDSVRGRFSISRDKAKNTVYLRMN | |
| | SLKPEDTAVYYCVADVSFGDLINGRSDYWGQGTQVTVSS | |
| 599 | EVQLVESGGGLVRAGDSLRLSCAASEGRTFTMRSVGWFRQAP | CD117 VHH (b26) |
| | GKEREFVAASNWWSSGSTMYAEYVKGRFTISRDNAKNTVYL | |
| | QMNSLKPEDTATYYCAASSTVYMSSRLSEYAYWGQGTQVTV | |
| | SS | |
| 600 | QVQLVESGGGLVRAGDSLRLSCAASEGRTFTMRSVGWFRQA | CD117 VHH (b27) |
| | PGKEREFVAASNWWSSGSTMYAEYVKGRFTISRDNAKNTVY | |
| | LQMNSLKPEDTATYYCAASSTVYMSSRLSEYAYWGQGTQVT | |
| | VSS | |
| 601 | EVQLVESGGGLVQTGGSLTLSCAASKRTLNTYAMGWFRQPP | CD117 VHH (b28) |
| | GKRREFVAADSWSGGSTMYAEYVKGRFTISRDNAKNTVYLQ | |
| | MNSLKPEDTATYYCAASSTVYMSSRLSEYAYWGQGTQVTVS | |
| | S | |
| 602 | EVQLVESGGGWVQTDDSLRLSCAASGGAFGNYAMGWLRQA | CD117 VHH (b29) |
| | PGKGREFVAGIDWSGGSTMYAEYVKGRFTISRDNAKNTVYL | |
| | QMNSLKPEDTATYYCAASSTVYMSSRLSEYAYWGQGTLVTV | |
| | SS | |
| 603 | QVQLVESGGGWVQTDDSLRLSCAASGGAFGNYAMGWLRQA | CD117 VHH (b30) |
| | PGKGREFVAGIDWSGGSTMYAEYVKGRFTISRDNAKNTVYL | |
| | QMNSLKPEDTATYYCAASSTVYMSSRLSEYAYWGQGTQVTV | |
| | SS | |
| 604 | QVQLVESGGGSVQTGDSLRLSCVASGSTFGSYAMAWYRQVP | CD117 VHH (b31) |
| | GKEREFVAAVRWSGGSTMYAEYVKGRFTISRDNAKNTVYLQ | |
| | MNGLKPEDTATYYCAASSTVYMSSRLSEYAYWGQGTLVTVS | |
| | S | |
| 605 | EVQLVESGGRLVQAGESLRLSCSASGGTFSSYLMAWFRQVPG | CD117 VHH (b32) |
| | KEREIVAFITWHGHTTYYTDSVKGRFAISRDNARNTMYLQMN | |
| | SLRPEDTAVYYCAADQRQEFSGTYSREYNYWGQGTLVTVSS | |
| 606 | QVQLVESGGRLVQAGESLRLSCSASGGTFSSYLMAWFRQVPG | CD117 VHH (b33) |
| | KEREIVAFITWHGHTTYYTDSVKGRFAISRDNARNTMYLQMN | |
| | SLRPEDTAVYYCAADQRQEFSGTYSREYNYWGQGTLVTVSS | |
| 607 | EVQLVESGGGLVQAGGSLGLACVASGDTLSTYFMGWFRQAP | CD117 VHH (b35) |
| | GKEREFVAAIGWSGWLTYYADSMKGRFTISRDNGKNSVYLQ | |
| | MNNLKRDDTAVYYCAAGKTFAAAPSTTLIGDYWGQGTLVTV | |
| | SS | |
| 608 | EVQLVESGGGLVQAGGSLRLACVASGDTLSTYFMGWFRQAP | CD117 VHH (b36) |
| | GKEREFVAAIGWSGWLTYYADSMKGRFTISRDNGKNSVYLQ | |
| | MNNLKRDDTAVYYCAAGKTFAAAPSTTLIGDYWGQGTQVT | |
| | VSS | |
| 609 | QVQLVESGGGLVQAGGSLRLSCVASGDTLSTYFMGWFRQAP | CD117 VHH (b37) |
| | GKEREFVAAIGWSGWLTYYADSMKGRFTISRDNGKNSVYLQ | |
| | MNNLKRDDTAVYYCAAGKTFAAAPSTTLIGDYWGQGTLVTV | |
| | SS | |
| 610 | EVQLVESGGGLVQVGGSLRLSCAASGRTTGRYFMGWFREAP | CD117 VHH (b38) |
| | GKEREFVSAIGWSVGSTYYVDSVKGRFTISRDNAKNTVYLQM | |
| | NSLKPEDTAVYYCAVGFEFSQGAGTAELIGDYWGQGTQVTV | |
| | SS | |
| 611 | EVQLVESGGGLVQVGGSLRLSCAASGRTTGRYFMGWFREAP | CD117 VHH (b39) |
| | GKEREFVSAIGWSVGSTYYVDSVKGRFTISRDNANNTVYLQM | |
| | NSLKPEDTAVYYCAVGFEFSQGAGTAELIGDYWGQGTQVTV | |
| | SS | |
| 612 | QVQLVESGGGLVQAGGSLTLSCAAPGRTFSNYFMGWFRQAP | CD117 VHH (b40) |
| | GKERDFVAAISWSGFLTYYADSVKGRFTISRDNAKNTVDLQM | |
| | NRLKPEDTAVYYCAMGSEFSPGAGTAELIGDYWGQGTLVTV | |
| | SS | |
| 613 | QVQLVESGGGLVQPGGSLRLSCAASGQTFTDYHHHVAWFRQ | CD117 VHH (b42) |
| | APGRERDFVAVISWSTGNTDYADSQRARFTISKDNAKNTAYL | |
| | QMNNLKFEDTAVYYCAARDYHPRFSYSADDYNYWGQGTQV | |
| | TVSS | |
| 614 | EVQLVESGGGLVQAGDSLRLSCTASISTSGNYATFKNYAMGW | CD117 VHH (b43) |
| | FRQAPGKAREFVAGISNSGGTTDYADSVKGRFTISRDNARKTT | |
| | YLQMNRLGPEDTAVYSCAADAWFGPPKRDPTTYSFRGPGTL | |
| | VTVSS | |
| 615 | EVQLVESGGGLVQAGDSLRLSCAVSGRTFSSVAMAWLRQGP | CD117 VHH (b44) |
| | GKAREFVASISWSGSTTDYADSVKGRFTISRDNAKKMVYLQM | |
| | NSLKPEDTAVYYCAAGRVPGSSPTIHNYVTWGQGTQVTVSS | |
| 616 | EVQLVESGGGSAHTGGPLRLSCVGTGPTTGHTFRGYAVAWFR | CD117 VHH (b45) |
| | QAPGKEREFVAAVRWNEGQDVYYADSVKGRFTISRDNDKNT | |
| | VYLQMNSLKPEDTAVYYCAAGVKWGSITFMASSSRYDFWGQ | |
| | GTQVTVSS | |
| 617 | EVQLVESGGGLVQAGGSLRLSCAASGRTFSYYIMGWFRQAPG | CD117 VHH (b47) |
| | KEREFVAAINRSGGTTYYADSVEGRFFISRDNAKNTMYLQMN | |
| | SLKPEDTAVYYCAAQPDSRGTYGYWGQGTLVTVSS | |
| 618 | EVQLVESGGGLVQAGGSLRLSCIDSGRTFSRYYMGWFRQAPG | CD117 VHH (b48) |
| | KEREFVAAISQNGYTKWYAESVKGRSAISRDIAKNTVDLQMN | |
| | SLKPEDTAVYYCAATSILEFSRDTASDFEYYGQGTLVTVSS | |
| 619 | EVQLVESGGGLVRAGDSLRLSCSASGRTFSKWTMAWFRQAP | CD117 VHH (b49) |
| | GKEREFVAGMSGDATYYAASVKGRFTSSRDNSKNTVYLQMD | |
| | SLKPEDTAVYFCAIYYGSGSYFQQNSYDTWGQGTQVTVSS | |
| 620 | QVQLVESGGGLVQAGGSLRLSCTASGRTESTYFMSWFRQTPG | CD117 VHH (b50) |
| | KERDFVAVINYSGDVTTYADSVKGRFTISRDNAKNTVHLQMN | |
| | SLTVEDTAVYYCAVHRTGPMNRADSYDYWGQGTLVTVSS | |
| 621 | EVQLVESGGGLVQAGGSLKLSCAASGNIANTYRMAWYGQAP | CD117 VHH (b51) |
| | GKQRELVANITSDGIASYADSVKGRFTISRDNAKNTVYLQMN | |
| | SLKAEDTAVYYCNIVYRRCSRGGYGCYSEYSWGQGTLVTVSS | |
| 622 | GFDDPRKAIELVQKRYGRPCDCSGGQVSEPPSDRVSQVTCSG | BaEV envelope |
| | KTAYLMPDQRWKCKSIPKDTSPSGPLQECPCNSYQSSVHSSCY | glycoprotein |
| | TSYQQCRSGNKTYYTATLLKTQTGGTSDVQVLGSTNKLIQSP | |
| | CNGIKGQSICWSTTAPIHVSDGGGPLDTTRIKSVQRKLEEIHKA | |
| | LYPELQYHPLAIPKVRDNLMVDAQTLNILNATYNLLLMSNTS | |
| | LVDDCWLCLKLGPPTPLAIPNFLLSYVTRSSDNISCLIIPPLLVQ | |
| | PMQFSNSSCLFSPSYNSTEEIDLGHVAFSNCTSITNVTGPICAV | |
| | NGSVFLCGNNMAYTYLPTNWTGLCVLATLLPDIDIIPGDEPVP | |
| | IPAIDHFIYRPKRAIQFIPLLAGLGITAAFTTGATGLGVSVTQYT | |
| | KLSNQLISDVQILSSTIQDLQDQVDSLAEVVLQNRRGLDLLTA | |
| | EQGGICLALQEKCCFYVNKSGIVRDKIKTLQEELERRRKDLAS | |
| | NPLWTGLQGLLPYLLPFLGPLLTLLLLLTIGPCIFNRLTAFINDK | |
| | LNIIHAMVLTQQYQVLRTDEEAQD | |
| 623 | VLTQQYQVLRTDEEAQD | partial fusion inhibitory |
| | | R peptide |
| 624 | NRLTAFINDKLNIIHAMVLTQQYQV | BaEV Cytoplasmic tail |
| 625 | GFDDPRKAIELVQKRYGRPCDCSGGQVSEPPSDRVSQVTCSG | Truncated BaEV |
| | KTAYLMPDQRWKCKSIPKDTSPSGPLQECPCNSYQSSVHSSCY | envelope glycoprotein |
| | TSYQQCRSGNKTYYTATLLKTQTGGTSDVQVLGSTNKLIQSP | |
| | CNGIKGQSICWSTTAPIHVSDGGGPLDTTRIKSVQRKLEEIHKA | |
| | LYPELQYHPLAIPKVRDNLMVDAQTLNILNATYNLLLMSNTS | |
| | LVDDCWLCLKLGPPTPLAIPNFLLSYVTRSSDNISCLIIPPLLVQ | |
| | PMQFSNSSCLFSPSYNSTEEIDLGHVAFSNCTSITNVTGPICAV | |
| | NGSVFLCGNNMAYTYLPTNWTGLCVLATLLPDIDIIPGDEPVP | |
| | IPAIDHFIYRPKRAIQFIPLLAGLGITAAFTTGATGLGVSVTQYT | |
| | KLSNQLISDVQILSSTIQDLQDQVDSLAEVVLQNRRGLDLLTA | |
| | EQGGICLALQEKCCFYVNKSGIVRDKIKTLQEELERRRKDLAS | |
| | NPLWTGLQGLLPYLLPFLGPLLTLLLLLTIGPCIFNRLTAFINDK | |
| | LNIIHAMVLTQQYQV | |
| 626 | ATGGAGACTGACACACTGCTCTTATGGGTGCTTCTGTTGTG | HA-CD3.s.001.01- |
| | GGTCCCCGGCAGCACCGGGGATTACCCCTACGATGTGCCTG | IgG1ser-PDGFR |
| | ACTATGCCGGAGGAAGTGCGGCTCAGCCGGCATCAGACAT | |
| | AAAACTCCAACAGTCAGGCGCTGAATTGGCTAGGCCAGGG | |
| | GCAAGCGTTAAGATGTCTTGTAAAACTTCCGGCTACACTTT | |
| | TACGCGGTACACCATGCACTGGGTCAAACAGCGACCCGGC | |
| | CAGGGGCTTGAGTGGATCGGCTACATTAACCCCTCGAGAG | |
| | GATACACCAACTACAATCAGAAGTTTAAGGACAAAGCCAC | |
| | TCTAACAACCGACAAGTCTTCCTCAACCGCATATATGCAGT | |
| | TGTCCAGCCTGACATCTGAGGATTCCGCCGTGTATTACTGC | |
| | GCACGTTATTATGACGATCATTATTGTCTTGACTACTGGGG | |
| | ACAGGGCACTACACTGACAGTGTCCTCTGTTGAGGGTGGA | |
| | GGCGGCTCTGGGGGGGGAGGGAGCGGAGGGGGTGGGAGC | |
| | GTGGACGATATCCAGCTCACCCAGAGTCCTGCCATTATGTC | |
| | TGCCAGTCCTGGAGAAAAGGTGACCATGACGTGCAGGGCT | |
| | AGCTCCTCAGTCAGCTACATGAACTGGTATCAACAGAAGTC | |
| | AGGCACTAGTCCCAAGCGCTGGATCTACGATACCTCCAAG | |
| | GTGGCCAGCGGAGTACCATATCGGTTCAGTGGCAGTGGTA | |
| | GCGGTACATCCTATTCGTTAACTATTTCAAGTATGGAAGCT | |
| | GAGGATGCCGCAACATACTACTGCCAACAATGGTCTAGCA | |
| | ATCCTCTGACCTTCGGCGCCGGTACGAAACTGGAGCTGAAA | |
| | GCGGCCGCTAGAGGGTCCGAGCCCAAGTCTAGCGACAAAA | |
| | CCCACACAAGTCCACCTTCCCCTGCCCCAGAAGCTGCAGGC | |
| | GGACCCAACGCTGTCGGCCAGGACACCCAAGAGGTGATAG | |
| | TGGTGCCACACAGCCTGCCCTTCAAGGTAGTGGTCATCTCC | |
| | GCAATCCTTGCCCTCGTTGTTTTGACAATTATTTCTCTGATT | |
| | ATCTTAATCATGCTGTGGCAGAAAAAGCCTAGGTGATAA | |
| 627 | ATGGAGACTGACACACTGCTCTTATGGGTGCTTCTGTTGTG | HA-CD3.s.001.01- |
| | GGTCCCCGGCAGCACCGGGGATTACCCCTACGATGTGCCTG | IgG1ser-GPI |
| | ACTATGCCGGAGGAAGTGCGGCTCAGCCGGCATCAGACAT | |
| | AAAACTCCAACAGTCAGGCGCTGAATTGGCTAGGCCAGGG | |
| | GCAAGCGTTAAGATGTCTTGTAAAACTTCCGGCTACACTTT | |
| | TACGCGGTACACCATGCACTGGGTCAAACAGCGACCCGGC | |
| | CAGGGGCTTGAGTGGATCGGCTACATTAACCCCTCGAGAG | |
| | GATACACCAACTACAATCAGAAGTTTAAGGACAAAGCCAC | |
| | TCTAACAACCGACAAGTCTTCCTCAACCGCATATATGCAGT | |
| | TGTCCAGCCTGACATCTGAGGATTCCGCCGTGTATTACTGC | |
| | GCACGTTATTATGACGATCATTATTGTCTTGACTACTGGGG | |
| | ACAGGGCACTACACTGACAGTGTCCTCTGTTGAGGGTGGA | |
| | GGCGGCTCTGGGGGGGGAGGGAGCGGAGGGGGTGGGAGC | |
| | GTGGACGATATCCAGCTCACCCAGAGTCCTGCCATTATGTC | |
| | TGCCAGTCCTGGAGAAAAGGTGACCATGACGTGCAGGGCT | |
| | AGCTCCTCAGTCAGCTACATGAACTGGTATCAACAGAAGTC | |
| | AGGCACTAGTCCCAAGCGCTGGATCTACGATACCTCCAAG | |
| | GTGGCCAGCGGAGTACCATATCGGTTCAGTGGCAGTGGTA | |
| | GCGGTACATCCTATTCGTTAACTATTTCAAGTATGGAAGCT | |
| | GAGGATGCCGCAACATACTACTGCCAACAATGGTCTAGCA | |
| | ATCCTCTGACCTTCGGCGCCGGTACGAAACTGGAGCTGAAA | |
| | GCGGCCGCTAGAGGGTCCGAGCCCAAGTCTAGCGACAAAA | |
| | CCCACACAAGTCCACCTTCCCCTGCCCCAGAAGCTGCAGGC | |
| | GGACCCCAGCTTGAGAACGGGGGCACATCTCTCTCCGAAA | |
| | AGACTGTCCTGTTACTGGTGACCCCTTTCTTGGCTGCCGCAT | |
| | GGAGCCTGCACCCCTGATAA | |
| 628 | ATGGAGACTGACACACTGCTCTTATGGGTGCTTCTGTTGTG | HA-CD3.s.001.01- |
| | GGTCCCCGGCAGCACCGGGGATTACCCCTACGATGTGCCTG | IgG1ser-VSVGan |
| | ACTATGCCGGAGGAAGTGCGGCTCAGCCGGCATCAGACAT | |
| | AAAACTCCAACAGTCAGGCGCTGAATTGGCTAGGCCAGGG | |
| | GCAAGCGTTAAGATGTCTTGTAAAACTTCCGGCTACACTTT | |
| | TACGCGGTACACCATGCACTGGGTCAAACAGCGACCCGGC | |
| | CAGGGGCTTGAGTGGATCGGCTACATTAACCCCTCGAGAG | |
| | GATACACCAACTACAATCAGAAGTTTAAGGACAAAGCCAC | |
| | TCTAACAACCGACAAGTCTTCCTCAACCGCATATATGCAGT | |
| | TGTCCAGCCTGACATCTGAGGATTCCGCCGTGTATTACTGC | |
| | GCACGTTATTATGACGATCATTATTGTCTTGACTACTGGGG | |
| | ACAGGGCACTACACTGACAGTGTCCTCTGTTGAGGGTGGA | |
| | GGCGGCTCTGGGGGGGGAGGGAGCGGAGGGGGTGGGAGC | |
| | GTGGACGATATCCAGCTCACCCAGAGTCCTGCCATTATGTC | |
| | TGCCAGTCCTGGAGAAAAGGTGACCATGACGTGCAGGGCT | |
| | AGCTCCTCAGTCAGCTACATGAACTGGTATCAACAGAAGTC | |
| | AGGCACTAGTCCCAAGCGCTGGATCTACGATACCTCCAAG | |
| | GTGGCCAGCGGAGTACCATATCGGTTCAGTGGCAGTGGTA | |
| | GCGGTACATCCTATTCGTTAACTATTTCAAGTATGGAAGCT | |
| | GAGGATGCCGCAACATACTACTGCCAACAATGGTCTAGCA | |
| | ATCCTCTGACCTTCGGCGCCGGTACGAAACTGGAGCTGAAA | |
| | GCGGCCGCTAGAGGGTCCGAGCCCAAGTCTAGCGACAAAA | |
| | CCCACACAAGTCCACCTTCCCCTGCCCCAGAAGCTGCAGGC | |
| | GGACCCTCCTCTATCGCCAGCTTCTTCTTCATCATCGGGCTG | |
| | ATTATCGGCCTGTTCCTGGTGCTGAGAGTGGGCATCCACCT | |
| | GTGCATCAAGCTGAAGCACACCAAGAAGCGGCAGATCTAC | |
| | ACCGACATCGAGATGAACCGGCTGGGCAAATGATAA | |
| 629 | ATGGAGACTGACACACTGCTCTTATGGGTGCTTCTGTTGTG | GGGGS |
| | GGTCCCCGGCAGCACCGGGGATTACCCCTACGATGTGCCTG | |
| | ACTATGCCGGAGGAAGTGCGGCTCAGCCGGCATCAGACAT | |
| | AAAACTCCAACAGTCAGGCGCTGAATTGGCTAGGCCAGGG | |
| | GCAAGCGTTAAGATGTCTTGTAAAACTTCCGGCTACACTTT | |
| | TACGCGGTACACCATGCACTGGGTCAAACAGCGACCCGGC | |
| | CAGGGGCTTGAGTGGATCGGCTACATTAACCCCTCGAGAG | |
| | GATACACCAACTACAATCAGAAGTTTAAGGACAAAGCCAC | |
| | TCTAACAACCGACAAGTCTTCCTCAACCGCATATATGCAGT | |
| | TGTCCAGCCTGACATCTGAGGATTCCGCCGTGTATTACTGC | |
| | GCACGTTATTATGACGATCATTATTGTCTTGACTACTGGGG | |
| | ACAGGGCACTACACTGACAGTGTCCTCTGTTGAGGGTGGA | |
| | GGCGGCTCTGGGGGGGGAGGGAGCGGAGGGGGGGGAGC | |
| | GTGGACGATATCCAGCTCACCCAGAGTCCTGCCATTATGTC | |
| | TGCCAGTCCTGGAGAAAAGGTGACCATGACGTGCAGGGCT | |
| | AGCTCCTCAGTCAGCTACATGAACTGGTATCAACAGAAGTC | |
| | AGGCACTAGTCCCAAGCGCTGGATCTACGATACCTCCAAG | |
| | GTGGCCAGCGGAGTACCATATCGGTTCAGTGGCAGTGGTA | |
| | GCGGTACATCCTATTCGTTAACTATTTCAAGTATGGAAGCT | |
| | GAGGATGCCGCAACATACTACTGCCAACAATGGTCTAGCA | |
| | ATCCTCTGACCTTCGGCGCCGGTACGAAACTGGAGCTGAAA | |
| | GCGGCCGCTAGAGGGTCCGAGCCCAAGTCTAGCGACAAAA | |
| | CCCACACAAGTCCACCTTCCCCTGCCCCAGAAGCTGCAGGC | |
| | GGACCCATTGCAAGTCAACCACTTTCTTTACGACCCGAGGC | |
| | CTGTAGGCCCGCTGCTGGGGGTGCTGTTCATACCAGGGGCC | |
| | TGGACTTCGCCTGCGACATATACATTTGGGCCCCTCTTGCA | |
| | GGCACTTGTGGAGTGCTGTTGTTATCCCTGGTCATCACCCT | |
| | ATATTGTAATCACCGTAATCGCCGGCGAGTGTGTAAATGCC | |
| | CTCGGCCAGTGGTTATCGCTAGCCAGCCGCTCAGCCTGAGG | |
| | CCAGAAGCGTGCCGCCCTGCAGCAGGAGGGGCCGTCCATA | |
| | CACGGGGCCTCGATTTTGCCTGTGATATTTATATCTGGGCC | |
| | CCCCTCGCGGGGACGTGCGGTGTCCTGTTGCTGTCACTGGT | |
| | GATCACACTCTACTGCAACCACAGAAACAGGCGCAGAGTG | |
| | TGTAAGTGCCCTAGACCCGTAGTGTGATAA | |
| 630 | ATGGAGACTGACACACTGCTCTTATGGGTGCTTCTGTTGTG | HA-CD3.s.001.01- |
| | GGTCCCCGGCAGCACCGGGGATTACCCCTACGATGTGCCTG | IgG1ser-ICAM1 |
| | ACTATGCCGGAGGAAGTGCGGCTCAGCCGGCATCAGACAT | |
| | AAAACTCCAACAGTCAGGCGCTGAATTGGCTAGGCCAGGG | |
| | GCAAGCGTTAAGATGTCTTGTAAAACTTCCGGCTACACTTT | |
| | TACGCGGTACACCATGCACTGGGTCAAACAGCGACCCGGC | |
| | CAGGGGCTTGAGTGGATCGGCTACATTAACCCCTCGAGAG | |
| | GATACACCAACTACAATCAGAAGTTTAAGGACAAAGCCAC | |
| | TCTAACAACCGACAAGTCTTCCTCAACCGCATATATGCAGT | |
| | TGTCCAGCCTGACATCTGAGGATTCCGCCGTGTATTACTGC | |
| | GCACGTTATTATGACGATCATTATTGTCTTGACTACTGGGG | |
| | ACAGGGCACTACACTGACAGTGTCCTCTGTTGAGGGTGGA | |
| | GGCGGCTCTGGGGGGGGAGGGAGCGGAGGGGGTGGGAGC | |
| | GTGGACGATATCCAGCTCACCCAGAGTCCTGCCATTATGTC | |
| | TGCCAGTCCTGGAGAAAAGGTGACCATGACGTGCAGGGCT | |
| | AGCTCCTCAGTCAGCTACATGAACTGGTATCAACAGAAGTC | |
| | AGGCACTAGTCCCAAGCGCTGGATCTACGATACCTCCAAG | |
| | GTGGCCAGCGGAGTACCATATCGGTTCAGTGGCAGTGGTA | |
| | GCGGTACATCCTATTCGTTAACTATTTCAAGTATGGAAGCT | |
| | GAGGATGCCGCAACATACTACTGCCAACAATGGTCTAGCA | |
| | ATCCTCTGACCTTCGGCGCCGGTACGAAACTGGAGCTGAAA | |
| | GCGGCCGCTAGAGGGTCCGAGCCCAAGTCTAGCGACAAAA | |
| | CCCACACAAGTCCACCTTCCCCTGCCCCAGAAGCTGCAGGC | |
| | GGACCCATAGTGATTATCACAGTTGTCGCCGCCGCTGTGAT | |
| | TATGGGAACCGCAGGCCTGAGCACTTATTTGTACAACCGGC | |
| | AGAGAAAGATCAAGAAATACAGGCTCTGATAA | |
| 631 | ATGGAGACTGACACACTGCTCTTATGGGTGCTTCTGTTGTG | HA-CD3.s.001.01- |
| | GGTCCCCGGCAGCACCGGGGATTACCCCTACGATGTGCCTG | IgG1ser-HLADRB |
| | ACTATGCCGGAGGAAGTGCGGCTCAGCCGGCATCAGACAT | |
| | AAAACTCCAACAGTCAGGCGCTGAATTGGCTAGGCCAGGG | |
| | GCAAGCGTTAAGATGTCTTGTAAAACTTCCGGCTACACTTT | |
| | TACGCGGTACACCATGCACTGGGTCAAACAGCGACCCGGC | |
| | CAGGGGCTTGAGTGGATCGGCTACATTAACCCCTCGAGAG | |
| | GATACACCAACTACAATCAGAAGTTTAAGGACAAAGCCAC | |
| | TCTAACAACCGACAAGTCTTCCTCAACCGCATATATGCAGT | |
| | TGTCCAGCCTGACATCTGAGGATTCCGCCGTGTATTACTGC | |
| | GCACGTTATTATGACGATCATTATTGTCTTGACTACTGGGG | |
| | ACAGGGCACTACACTGACAGTGTCCTCTGTTGAGGGTGGA | |
| | GGCGGCTCTGGGGGGGGAGGGAGCGGAGGGGGTGGGAGC | |
| | GTGGACGATATCCAGCTCACCCAGAGTCCTGCCATTATGTC | |
| | TGCCAGTCCTGGAGAAAAGGTGACCATGACGTGCAGGGCT | |
| | AGCTCCTCAGTCAGCTACATGAACTGGTATCAACAGAAGTC | |
| | AGGCACTAGTCCCAAGCGCTGGATCTACGATACCTCCAAG | |
| | GTGGCCAGCGGAGTACCATATCGGTTCAGTGGCAGTGGTA | |
| | GCGGTACATCCTATTCGTTAACTATTTCAAGTATGGAAGCT | |
| | GAGGATGCCGCAACATACTACTGCCAACAATGGTCTAGCA | |
| | ATCCTCTGACCTTCGGCGCCGGTACGAAACTGGAGCTGAAA | |
| | GCGGCCGCTAGAGGGTCCGAGCCCAAGTCTAGCGACAAAA | |
| | CCCACACAAGTCCACCTTCCCCTGCCCCAGAAGCTGCAGGC | |
| | GGACCCATGCTGTCTGGTGTCGGAGGTTTTGTGCTCGGACT | |
| | CTTGTTTCTGGGGGCCGGCCTTTTCATCTACTTCAGGAACC | |
| | AGAAGGGCCACAGCGGGTTACAACCCACCGGCTTCCTGTC | |
| | CTGATAA | |
| 632 | ATGGAGACTGACACACTGCTCTTATGGGTGCTTCTGTTGTG | HA-CD3.s.001.01- |
| | GGTCCCCGGCAGCACCGGGGATTACCCCTACGATGTGCCTG | IgG1ser-CD49d |
| | ACTATGCCGGAGGAAGTGCGGCTCAGCCGGCATCAGACAT | |
| | AAAACTCCAACAGTCAGGCGCTGAATTGGCTAGGCCAGGG | |
| | GCAAGCGTTAAGATGTCTTGTAAAACTTCCGGCTACACTTT | |
| | TACGCGGTACACCATGCACTGGGTCAAACAGCGACCCGGC | |
| | CAGGGGCTTGAGTGGATCGGCTACATTAACCCCTCGAGAG | |
| | GATACACCAACTACAATCAGAAGTTTAAGGACAAAGCCAC | |
| | TCTAACAACCGACAAGTCTTCCTCAACCGCATATATGCAGT | |
| | TGTCCAGCCTGACATCTGAGGATTCCGCCGTGTATTACTGC | |
| | GCACGTTATTATGACGATCATTATTGTCTTGACTACTGGGG | |
| | ACAGGGCACTACACTGACAGTGTCCTCTGTTGAGGGTGGA | |
| | GGCGGCTCTGGGGGGGGAGGGAGCGGAGGGGGTGGGAGC | |
| | GTGGACGATATCCAGCTCACCCAGAGTCCTGCCATTATGTC | |
| | TGCCAGTCCTGGAGAAAAGGTGACCATGACGTGCAGGGCT | |
| | AGCTCCTCAGTCAGCTACATGAACTGGTATCAACAGAAGTC | |
| | AGGCACTAGTCCCAAGCGCTGGATCTACGATACCTCCAAG | |
| | GTGGCCAGCGGAGTACCATATCGGTTCAGTGGCAGTGGTA | |
| | GCGGTACATCCTATTCGTTAACTATTTCAAGTATGGAAGCT | |
| | GAGGATGCCGCAACATACTACTGCCAACAATGGTCTAGCA | |
| | ATCCTCTGACCTTCGGCGCCGGTACGAAACTGGAGCTGAAA | |
| | GCGGCCGCTAGAGGGTCCGAGCCCAAGTCTAGCGACAAAA | |
| | CCCACACAAGTCCACCTTCCCCTGCCCCAGAAGCTGCAGGC | |
| | GGACCCATAGTGATCATCAGCTCCAGTCTGCTGTTAGGGCT | |
| | CATTGTTTTGCTTCTGATTTCTTATGTCATGTGGAAAGCCGG | |
| | CTTTTTCAAGAGGCAGTACAAGTCATGATAA | |
| 633 | ATGATGGACCAAGCCCGCAGCGCCTTTAGTAATCTGTTCGG | TFR1-IgG1ser- |
| | CGGGGAACCCTTATCCTATACCAGGTTCAGCTTGGCTAGAC | CD3.s.001.01-HA |
| | AGGTTGATGGCGACAACTCACACGTCGAAATGAAACTCGC | |
| | CGTGGACGAGGAGGAGAATGCAGATAATAACACAAAGGC | |
| | GAACGTGACGAAACCTAAGCGGTGTTCCGGATCTATTTGCT | |
| | ACGGTACTATAGCTGTAATTGTGTTCTTTCTTATCGGATTTA | |
| | TGATCGGCTACCTGGGGGAGCCCAAGTCTAGCGACAAAAC | |
| | CCACACAAGTCCACCTTCCCCTGCCCCAGAAGCTGCAGGCG | |
| | GACCCGCGGCTCAGCCGGCATCAGACATAAAACTCCAACA | |
| | GTCAGGCGCTGAATTGGCTAGGCCAGGGGCAAGCGTTAAG | |
| | ATGTCTTGTAAAACTTCCGGCTACACTTTTACGCGGTACAC | |
| | CATGCACTGGGTCAAACAGCGACCCGGCCAGGGGCTTGAG | |
| | TGGATCGGCTACATTAACCCCTCGAGAGGATACACCAACTA | |
| | CAATCAGAAGTTTAAGGACAAAGCCACTCTAACAACCGAC | |
| | AAGTCTTCCTCAACCGCATATATGCAGTIGTCCAGCCTGAC | |
| | ATCTGAGGATTCCGCCGTGTATTACTGCGCACGTTATTATG | |
| | ACGATCATTATTGTCTTGACTACTGGGGACAGGGCACTACA | |
| | CTGACAGTGTCCTCTGTTGAGGGTGGAGGCGGCTCTGGGGG | |
| | GGGAGGGAGCGGAGGGGGTGGGAGCGTGGACGATATCCA | |
| | GCTCACCCAGAGTCCTGCCATTATGTCTGCCAGTCCTGGAG | |
| | AAAAGGTGACCATGACGTGCAGGGCTAGCTCCTCAGTCAG | |
| | CTACATGAACTGGTATCAACAGAAGTCAGGCACTAGTCCC | |
| | AAGCGCTGGATCTACGATACCTCCAAGGTGGCCAGCGGAG | |
| | TACCATATCGGTTCAGTGGCAGTGGTAGCGGTACATCCTAT | |
| | TCGTTAACTATTTCAAGTATGGAAGCTGAGGATGCCGCAAC | |
| | ATACTACTGCCAACAATGGTCTAGCAATCCTCTGACCTTCG | |
| | GCGCCGGTACGAAACTGGAGCTGAAAGCGGCCGCTAGAGG | |
| | GTCCGGAGGAAGTTACCCCTACGATGTGCCTGACTATGCCT | |
| | GATAA | |
| 634 | ATGGAGACTGACACACTGCTCTTATGGGTGCTTCTGTTGTG | HA-CD3.s.001.01- |
| | GGTCCCCGGCAGCACCGGGGATTACCCCTACGATGTGCCTG | EAAAKx3-PDGFR |
| | ACTATGCCGGAGGAAGTGCGGCTCAGCCGGCATCAGACAT | |
| | AAAACTCCAACAGTCAGGCGCTGAATTGGCTAGGCCAGGG | |
| | GCAAGCGTTAAGATGTCTTGTAAAACTTCCGGCTACACTTT | |
| | TACGCGGTACACCATGCACTGGGTCAAACAGCGACCCGGC | |
| | CAGGGGCTTGAGTGGATCGGCTACATTAACCCCTCGAGAG | |
| | GATACACCAACTACAATCAGAAGTTTAAGGACAAAGCCAC | |
| | TCTAACAACCGACAAGTCTTCCTCAACCGCATATATGCAGT | |
| | TGTCCAGCCTGACATCTGAGGATTCCGCCGTGTATTACTGC | |
| | GCACGTTATTATGACGATCATTATTGTCTTGACTACTGGGG | |
| | ACAGGGCACTACACTGACAGTGTCCTCTGTTGAGGGTGGA | |
| | GGCGGCTCTGGGGGGGGAGGGAGCGGAGGGGGTGGGAGC | |
| | GTGGACGATATCCAGCTCACCCAGAGTCCTGCCATTATGTC | |
| | TGCCAGTCCTGGAGAAAAGGTGACCATGACGTGCAGGGCT | |
| | AGCTCCTCAGTCAGCTACATGAACTGGTATCAACAGAAGTC | |
| | AGGCACTAGTCCCAAGCGCTGGATCTACGATACCTCCAAG | |
| | GTGGCCAGCGGAGTACCATATCGGTTCAGTGGCAGTGGTA | |
| | GCGGTACATCCTATTCGTTAACTATTTCAAGTATGGAAGCT | |
| | GAGGATGCCGCAACATACTACTGCCAACAATGGTCTAGCA | |
| | ATCCTCTGACCTTCGGCGCCGGTACGAAACTGGAGCTGAAA | |
| | GCGGCCGCTAGAGGGTCCGCGGAGGCTGCTGCTAAAGAGG | |
| | CCGCCGCCAAGGAAGCAGCAGCCAAGGCAAACGCTGTCGG | |
| | CCAGGACACCCAAGAGGTGATAGTGGTGCCACACAGCCTG | |
| | CCCTTCAAGGTAGTGGTCATCTCCGCAATCCTTGCCCTCGTT | |
| | GTTTTGACAATTATTTCTCTGATTATCTTAATCATGCTGTGG | |
| | CAGAAAAAGCCTAGGTGATAA | |
| 635 | ATGGAGACTGACACACTGCTCTTATGGGTGCTTCTGTTGTG | HA-CD3.s.001.01- |
| | GGTCCCCGGCAGCACCGGGGATTACCCCTACGATGTGCCTG | EAAAKx3-GPI |
| | ACTATGCCGGAGGAAGTGCGGCTCAGCCGGCATCAGACAT | |
| | AAAACTCCAACAGTCAGGCGCTGAATTGGCTAGGCCAGGG | |
| | GCAAGCGTTAAGATGTCTTGTAAAACTTCCGGCTACACTTT | |
| | TACGCGGTACACCATGCACTGGGTCAAACAGCGACCCGGC | |
| | CAGGGGCTTGAGTGGATCGGCTACATTAACCCCTCGAGAG | |
| | GATACACCAACTACAATCAGAAGTTTAAGGACAAAGCCAC | |
| | TCTAACAACCGACAAGTCTTCCTCAACCGCATATATGCAGT | |
| | TGTCCAGCCTGACATCTGAGGATTCCGCCGTGTATTACTGC | |
| | GCACGTTATTATGACGATCATTATTGTCTTGACTACTGGGG | |
| | ACAGGGCACTACACTGACAGTGTCCTCTGTTGAGGGTGGA | |
| | GGCGGCTCTGGGGGGGGAGGGAGCGGAGGGGGTGGGAGC | |
| | GTGGACGATATCCAGCTCACCCAGAGTCCTGCCATTATGTC | |
| | TGCCAGTCCTGGAGAAAAGGTGACCATGACGTGCAGGGCT | |
| | AGCTCCTCAGTCAGCTACATGAACTGGTATCAACAGAAGTC | |
| | AGGCACTAGTCCCAAGCGCTGGATCTACGATACCTCCAAG | |
| | GTGGCCAGCGGAGTACCATATCGGTTCAGTGGCAGTGGTA | |
| | GCGGTACATCCTATTCGTTAACTATTTCAAGTATGGAAGCT | |
| | GAGGATGCCGCAACATACTACTGCCAACAATGGTCTAGCA | |
| | ATCCTCTGACCTTCGGCGCCGGTACGAAACTGGAGCTGAAA | |
| | GCGGCCGCTAGAGGGTCCGCGGAGGCTGCTGCTAAAGAGG | |
| | CCGCCGCCAAGGAAGCAGCAGCCAAGGCACAGCTTGAGAA | |
| | CGGGGGCACATCTCTCTCCGAAAAGACTGTCCTGTTACTGG | |
| | TGACCCCTTTCTTGGCTGCCGCATGGAGCCTGCACCCCTGA | |
| | TAA | |
| 636 | ATGGAGACTGACACACTGCTCTTATGGGTGCTTCTGTTGTG | HA-CD3.s.001.01- |
| | GGTCCCCGGCAGCACCGGGGATTACCCCTACGATGTGCCTG | EAAAKx3-VSVGan |
| | ACTATGCCGGAGGAAGTGCGGCTCAGCCGGCATCAGACAT | |
| | AAAACTCCAACAGTCAGGCGCTGAATTGGCTAGGCCAGGG | |
| | GCAAGCGTTAAGATGTCTTGTAAAACTTCCGGCTACACTTT | |
| | TACGCGGTACACCATGCACTGGGTCAAACAGCGACCCGGC | |
| | CAGGGGCTTGAGTGGATCGGCTACATTAACCCCTCGAGAG | |
| | GATACACCAACTACAATCAGAAGTTTAAGGACAAAGCCAC | |
| | TCTAACAACCGACAAGTCTTCCTCAACCGCATATATGCAGT | |
| | TGTCCAGCCTGACATCTGAGGATTCCGCCGTGTATTACTGC | |
| | GCACGTTATTATGACGATCATTATTGTCTTGACTACTGGGG | |
| | ACAGGGCACTACACTGACAGTGTCCTCTGTTGAGGGTGGA | |
| | GGCGGCTCTGGGGGGGGAGGGAGCGGAGGGGGTGGGAGC | |
| | GTGGACGATATCCAGCTCACCCAGAGTCCTGCCATTATGTC | |
| | TGCCAGTCCTGGAGAAAAGGTGACCATGACGTGCAGGGCT | |
| | AGCTCCTCAGTCAGCTACATGAACTGGTATCAACAGAAGTC | |
| | AGGCACTAGTCCCAAGCGCTGGATCTACGATACCTCCAAG | |
| | GTGGCCAGCGGAGTACCATATCGGTTCAGTGGCAGTGGTA | |
| | GCGGTACATCCTATTCGTTAACTATTTCAAGTATGGAAGCT | |
| | GAGGATGCCGCAACATACTACTGCCAACAATGGTCTAGCA | |
| | ATCCTCTGACCTTCGGCGCCGGTACGAAACTGGAGCTGAAA | |
| | GCGGCCGCTAGAGGGTCCGCGGAGGCTGCTGCTAAAGAGG | |
| | CCGCCGCCAAGGAAGCAGCAGCCAAGGCATCCTCTATCGC | |
| | CAGCTTCTTCTTCATCATCGGGCTGATTATCGGCCTGTTCCT | |
| | GGTGCTGAGAGTGGGCATCCACCTGTGCATCAAGCTGAAG | |
| | CACACCAAGAAGCGGCAGATCTACACCGACATCGAGATGA | |
| | ACCGGCTGGGCAAATGATAA | |
| 637 | ATGGAGACTGACACACTGCTCTTATGGGTGCTTCTGTTGTG | HA-CD3.s.001.01- |
| | GGTCCCCGGCAGCACCGGGGATTACCCCTACGATGTGCCTG | EAAAKx3-CD8αTm |
| | ACTATGCCGGAGGAAGTGCGGCTCAGCCGGCATCAGACAT | |
| | AAAACTCCAACAGTCAGGCGCTGAATTGGCTAGGCCAGGG | |
| | GCAAGCGTTAAGATGTCTTGTAAAACTTCCGGCTACACTTT | |
| | TACGCGGTACACCATGCACTGGGTCAAACAGCGACCCGGC | |
| | CAGGGGCTTGAGTGGATCGGCTACATTAACCCCTCGAGAG | |
| | GATACACCAACTACAATCAGAAGTTTAAGGACAAAGCCAC | |
| | TCTAACAACCGACAAGTCTTCCTCAACCGCATATATGCAGT | |
| | TGTCCAGCCTGACATCTGAGGATTCCGCCGTGTATTACTGC | |
| | GCACGTTATTATGACGATCATTATTGTCTTGACTACTGGGG | |
| | ACAGGGCACTACACTGACAGTGTCCTCTGTTGAGGGTGGA | |
| | GGCGGCTCTGGGGGGGGAGGGAGCGGAGGGGGTGGGAGC | |
| | GTGGACGATATCCAGCTCACCCAGAGTCCTGCCATTATGTC | |
| | TGCCAGTCCTGGAGAAAAGGTGACCATGACGTGCAGGGCT | |
| | AGCTCCTCAGTCAGCTACATGAACTGGTATCAACAGAAGTC | |
| | AGGCACTAGTCCCAAGCGCTGGATCTACGATACCTCCAAG | |
| | GTGGCCAGCGGAGTACCATATCGGTTCAGTGGCAGTGGTA | |
| | GCGGTACATCCTATTCGTTAACTATTTCAAGTATGGAAGCT | |
| | GAGGATGCCGCAACATACTACTGCCAACAATGGTCTAGCA | |
| | ATCCTCTGACCTTCGGCGCCGGTACGAAACTGGAGCTGAAA | |
| | GCGGCCGCTAGAGGGTCCGCGGAGGCTGCTGCTAAAGAGG | |
| | CCGCCGCCAAGGAAGCAGCAGCCAAGGCAATTGCAAGTCA | |
| | ACCACTTTCTTTACGACCCGAGGCCTGTAGGCCCGCTGCTG | |
| | GGGGTGCTGTTCATACCAGGGGCCTGGACTTCGCCTGCGAC | |
| | ATATACATTTGGGCCCCTCTTGCAGGCACTTGTGGAGTGCT | |
| | GTTGTTATCCCTGGTCATCACCCTATATTGTAATCACCGTAA | |
| | TCGCCGGCGAGTGTGTAAATGCCCTCGGCCAGTGGTTATCG | |
| | CTAGCCAGCCGCTCAGCCTGAGGCCAGAAGCGTGCCGCCC | |
| | TGCAGCAGGAGGGGCCGTCCATACACGGGGCCTCGATTTT | |
| | GCCTGTGATATTTATATCTGGGCCCCCCTCGCGGGGACGTG | |
| | CGGTGTCCTGTTGCTGTCACTGGTGATCACACTCTACTGCA | |
| | ACCACAGAAACAGGCGCAGAGTGTGTAAGTGCCCTAGACC | |
| | CGTAGTGTGATAA | |
| 638 | ATGGAGACTGACACACTGCTCTTATGGGTGCTTCTGTTGTG | HA-CD3.s.001.01- |
| | GGTCCCCGGCAGCACCGGGGATTACCCCTACGATGTGCCTG | EAAAKx3-HLADRB |
| | ACTATGCCGGAGGAAGTGCGGCTCAGCCGGCATCAGACAT | |
| | AAAACTCCAACAGTCAGGCGCTGAATTGGCTAGGCCAGGG | |
| | GCAAGCGTTAAGATGTCTTGTAAAACTTCCGGCTACACTTT | |
| | TACGCGGTACACCATGCACTGGGTCAAACAGCGACCCGGC | |
| | CAGGGGCTTGAGTGGATCGGCTACATTAACCCCTCGAGAG | |
| | GATACACCAACTACAATCAGAAGTTTAAGGACAAAGCCAC | |
| | TCTAACAACCGACAAGTCTTCCTCAACCGCATATATGCAGT | |
| | TGTCCAGCCTGACATCTGAGGATTCCGCCGTGTATTACTGC | |
| | GCACGTTATTATGACGATCATTATTGTCTTGACTACTGGGG | |
| | ACAGGGCACTACACTGACAGTGTCCTCTGTTGAGGGTGGA | |
| | GGCGGCTCTGGGGGGGGAGGGAGCGGAGGGGGTGGGAGC | |
| | GTGGACGATATCCAGCTCACCCAGAGTCCTGCCATTATGTC | |
| | TGCCAGTCCTGGAGAAAAGGTGACCATGACGTGCAGGGCT | |
| | AGCTCCTCAGTCAGCTACATGAACTGGTATCAACAGAAGTC | |
| | AGGCACTAGTCCCAAGCGCTGGATCTACGATACCTCCAAG | |
| | GTGGCCAGCGGAGTACCATATCGGTTCAGTGGCAGTGGTA | |
| | GCGGTACATCCTATTCGTTAACTATTTCAAGTATGGAAGCT | |
| | GAGGATGCCGCAACATACTACTGCCAACAATGGTCTAGCA | |
| | ATCCTCTGACCTTCGGCGCCGGTACGAAACTGGAGCTGAAA | |
| | GCGGCCGCTAGAGGGTCCGCGGAGGCTGCTGCTAAAGAGG | |
| | CCGCCGCCAAGGAAGCAGCAGCCAAGGCAATGCTGTCTGG | |
| | TGTCGGAGGTTTTGTGCTCGGACTCTTGTTTCTGGGGGCCG | |
| | GCCTTTTCATCTACTTCAGGAACCAGAAGGGCCACAGCGGG | |
| | TTACAACCCACCGGCTTCCTGTCCTGATAA | |
| 639 | ATGGAGACTGACACACTGCTCTTATGGGTGCTTCTGTTGTG | HA-CD3.s.001.01- |
| | GGTCCCCGGCAGCACCGGGGATTACCCCTACGATGTGCCTG | EAAAKx3-CD49d |
| | ACTATGCCGGAGGAAGTGCGGCTCAGCCGGCATCAGACAT | |
| | AAAACTCCAACAGTCAGGCGCTGAATTGGCTAGGCCAGGG | |
| | GCAAGCGTTAAGATGTCTTGTAAAACTTCCGGCTACACTTT | |
| | TACGCGGTACACCATGCACTGGGTCAAACAGCGACCCGGC | |
| | CAGGGGCTTGAGTGGATCGGCTACATTAACCCCTCGAGAG | |
| | GATACACCAACTACAATCAGAAGTTTAAGGACAAAGCCAC | |
| | TCTAACAACCGACAAGTCTTCCTCAACCGCATATATGCAGT | |
| | TGTCCAGCCTGACATCTGAGGATTCCGCCGTGTATTACTGC | |
| | GCACGTTATTATGACGATCATTATTGTCTTGACTACTGGGG | |
| | ACAGGGCACTACACTGACAGTGTCCTCTGTTGAGGGTGGA | |
| | GGCGGCTCTGGGGGGGGAGGGAGCGGAGGGGGTGGGAGC | |
| | GTGGACGATATCCAGCTCACCCAGAGTCCTGCCATTATGTC | |
| | TGCCAGTCCTGGAGAAAAGGTGACCATGACGTGCAGGGCT | |
| | AGCTCCTCAGTCAGCTACATGAACTGGTATCAACAGAAGTC | |
| | AGGCACTAGTCCCAAGCGCTGGATCTACGATACCTCCAAG | |
| | GTGGCCAGCGGAGTACCATATCGGTTCAGTGGCAGTGGTA | |
| | GCGGTACATCCTATTCGTTAACTATTTCAAGTATGGAAGCT | |
| | GAGGATGCCGCAACATACTACTGCCAACAATGGTCTAGCA | |
| | ATCCTCTGACCTTCGGCGCCGGTACGAAACTGGAGCTGAAA | |
| | GCGGCCGCTAGAGGGTCCGCGGAGGCTGCTGCTAAAGAGG | |
| | CCGCCGCCAAGGAAGCAGCAGCCAAGGCAATAGTGATCAT | |
| | CAGCTCCAGTCTGCTGTTAGGGCTCATTGTTTTGCTTCTGAT | |
| | TTCTTATGTCATGTGGAAAGCCGGCTTTTTCAAGAGGCAGT | |
| | ACAAGTCATGATAA | |
| 640 | ATGATGGACCAAGCCCGCAGCGCCTTTAGTAATCTGTTCGG | TFR1-EAAAKx3- |
| | CGGGGAACCCTTATCCTATACCAGGTTCAGCTTGGCTAGAC | CD3.s.001.01-HA |
| | AGGTTGATGGCGACAACTCACACGTCGAAATGAAACTCGC | |
| | CGTGGACGAGGAGGAGAATGCAGATAATAACACAAAGGC | |
| | GAACGTGACGAAACCTAAGCGGTGTTCCGGATCTATTTGCT | |
| | ACGGTACTATAGCTGTAATTGTGTTCTTTCTTATCGGATTTA | |
| | TGATCGGCTACCTGGGGGCGGAGGCTGCTGCTAAAGAGGC | |
| | CGCCGCCAAGGAAGCAGCAGCCAAGGCAGCGGCTCAGCCG | |
| | GCATCAGACATAAAACTCCAACAGTCAGGCGCTGAATTGG | |
| | CTAGGCCAGGGGCAAGCGTTAAGATGTCTTGTAAAACTTCC | |
| | GGCTACACTTTTACGCGGTACACCATGCACTGGGTCAAACA | |
| | GCGACCCGGCCAGGGGCTTGAGTGGATCGGCTACATTAAC | |
| | CCCTCGAGAGGATACACCAACTACAATCAGAAGTTTAAGG | |
| | ACAAAGCCACTCTAACAACCGACAAGTCTTCCTCAACCGCA | |
| | TATATGCAGTTGTCCAGCCTGACATCTGAGGATTCCGCCGT | |
| | GTATTACTGCGCACGTTATTATGACGATCATTATTGTCTTGA | |
| | CTACTGGGGACAGGGCACTACACTGACAGTGTCCTCTGTTG | |
| | AGGGTGGAGGCGGCTCTGGGGGGGGAGGGAGCGGAGGGG | |
| | GTGGGAGCGTGGACGATATCCAGCTCACCCAGAGTCCTGC | |
| | CATTATGTCTGCCAGTCCTGGAGAAAAGGTGACCATGACGT | |
| | GCAGGGCTAGCTCCTCAGTCAGCTACATGAACTGGTATCAA | |
| | CAGAAGTCAGGCACTAGTCCCAAGCGCTGGATCTACGATA | |
| | CCTCCAAGGTGGCCAGCGGAGTACCATATCGGTTCAGTGGC | |
| | AGTGGTAGCGGTACATCCTATTCGTTAACTATTTCAAGTAT | |
| | GGAAGCTGAGGATGCCGCAACATACTACTGCCAACAATGG | |
| | TCTAGCAATCCTCTGACCTTCGGCGCCGGTACGAAACTGGA | |
| | GCTGAAAGCGGCCGCTAGAGGGTCCGGAGGAAGTTACCCC | |
| | TACGATGTGCCTGACTATGCCTGATAA | |
| 641 | ATGGAGACTGACACACTGCTCTTATGGGTGCTTCTGTTGTG | HA-CD3.s.004.01- |
| | GGTCCCCGGCAGCACCGGGGATTACCCCTACGATGTGCCTG | IgG1ser-PDGFR |
| | ACTATGCCGGAGGAAGTCAGGTGCAGCTGCAGCAGAGCGG | |
| | AGCCGAGCTGGCACGTCCAGGCGCCAGTGTGAAGATGTCC | |
| | TGTAAAGCGTCAGGGTACACCTTCACCAGATACACCATGCA | |
| | TTGGGTTAAACAGCGCCCCGGCCAGGGGCTTGAATGGATT | |
| | GGCTACATAAACCCTTCAAGGGGTTATACTAACTATAACCA | |
| | AAAGTTCAAAGACAAAGCTACACTGACAACTGACAAGTCA | |
| | AGTAGCACCGCTTACATGCAGCTTTCATCTCTGACCTCAGA | |
| | GGATAGTGCAGTCTATTATTGCGCGCGATATTACGACGATC | |
| | ACTACAGCCTCGATTACTGGGGACAAGGAACGACATTGAC | |
| | TGTATCGTCCGGGGGCGGTGGGTCTGGCGGAGGCGGGAGT | |
| | GGAGGAGGCGGTAGCGACATTGTTTTAACGCAAAGCCCTG | |
| | CTATCATCGTGATTTCCGCCTCCCCAGGCGAGAAAGTCACT | |
| | ATGACCTGCTCGGCCAGTTCCTCTGTGTCTTACATGAATTG | |
| | GTATCAGCAGAAGTCCGGCACCTCTCCGAAAAGGTGGATC | |
| | TATGATACTTCCAAGCTGGCCTCTGGCGTGCCCGCACACTT | |
| | TCGGGGATCAGGGTCCGGGACAAGCTACAGCCTAACAATA | |
| | AGCGGTATGGAAGCTGAGGACGCAGCCACCTACTATTGTC | |
| | AGCAATGGAGCAGTAATCCCTTCACTTTTGGGTCTGGTACA | |
| | AAGCTCGAAATCAAGGAGCCCAAGTCTAGCGACAAAACCC | |
| | ACACAAGTCCACCTTCCCCTGCCCCAGAAGCTGCAGGCGG | |
| | ACCCAACGCTGTCGGCCAGGACACCCAAGAGGTGATAGTG | |
| | GTGCCACACAGCCTGCCCTTCAAGGTAGTGGTCATCTCCGC | |
| | AATCCTTGCCCTCGTTGTTTTGACAATTATTTCTCTGATTAT | |
| | CTTAATCATGCTGTGGCAGAAAAAGCCTAGGTGATAA | |
| 642 | ATGGAGACTGACACACTGCTCTTATGGGTGCTTCTGTTGTG | HA-CD3.s.004.01- |
| | GGTCCCCGGCAGCACCGGGGATTACCCCTACGATGTGCCTG | IgG1ser-GPI |
| | ACTATGCCGGAGGAAGTCAGGTGCAGCTGCAGCAGAGCGG | |
| | AGCCGAGCTGGCACGTCCAGGCGCCAGTGTGAAGATGTCC | |
| | TGTAAAGCGTCAGGGTACACCTTCACCAGATACACCATGCA | |
| | TTGGGTTAAACAGCGCCCCGGCCAGGGGCTTGAATGGATT | |
| | GGCTACATAAACCCTTCAAGGGGTTATACTAACTATAACCA | |
| | AAAGTTCAAAGACAAAGCTACACTGACAACTGACAAGTCA | |
| | AGTAGCACCGCTTACATGCAGCTTTCATCTCTGACCTCAGA | |
| | GGATAGTGCAGTCTATTATTGCGCGCGATATTACGACGATC | |
| | ACTACAGCCTCGATTACTGGGGACAAGGAACGACATTGAC | |
| | TGTATCGTCCGGGGGCGGTGGGTCTGGCGGAGGCGGGAGT | |
| | GGAGGAGGCGGTAGCGACATTGTTTTAACGCAAAGCCCTG | |
| | CTATCATCGTGATTTCCGCCTCCCCAGGCGAGAAAGTCACT | |
| | ATGACCTGCTCGGCCAGTTCCTCTGTGTCTTACATGAATTG | |
| | GTATCAGCAGAAGTCCGGCACCTCTCCGAAAAGGTGGATC | |
| | TATGATACTTCCAAGCTGGCCTCTGGCGTGCCCGCACACTT | |
| | TCGGGGATCAGGGTCCGGGACAAGCTACAGCCTAACAATA | |
| | AGCGGTATGGAAGCTGAGGACGCAGCCACCTACTATTGTC | |
| | AGCAATGGAGCAGTAATCCCTTCACTTTTGGGTCTGGTACA | |
| | AAGCTCGAAATCAAGGAGCCCAAGTCTAGCGACAAAACCC | |
| | ACACAAGTCCACCTTCCCCTGCCCCAGAAGCTGCAGGCGG | |
| | ACCCCAGCTTGAGAACGGGGGCACATCTCTCTCCGAAAAG | |
| | ACTGTCCTGTTACTGGTGACCCCTTTCTTGGCTGCCGCATGG | |
| | AGCCTGCACCCCTGATAA | |
| 643 | ATGGAGACTGACACACTGCTCTTATGGGTGCTTCTGTTGTG | HA-CD3.s.004.01- |
| | GGTCCCCGGCAGCACCGGGGATTACCCCTACGATGTGCCTG | IgG1ser-VSVGan |
| | ACTATGCCGGAGGAAGTCAGGTGCAGCTGCAGCAGAGCGG | |
| | AGCCGAGCTGGCACGTCCAGGCGCCAGTGTGAAGATGTCC | |
| | TGTAAAGCGTCAGGGTACACCTTCACCAGATACACCATGCA | |
| | TTGGGTTAAACAGCGCCCCGGCCAGGGGCTTGAATGGATT | |
| | GGCTACATAAACCCTTCAAGGGGTTATACTAACTATAACCA | |
| | AAAGTTCAAAGACAAAGCTACACTGACAACTGACAAGTCA | |
| | AGTAGCACCGCTTACATGCAGCTTTCATCTCTGACCTCAGA | |
| | GGATAGTGCAGTCTATTATTGCGCGCGATATTACGACGATC | |
| | ACTACAGCCTCGATTACTGGGGACAAGGAACGACATTGAC | |
| | TGTATCGTCCGGGGGCGGTGGGTCTGGCGGAGGCGGGAGT | |
| | GGAGGAGGCGGTAGCGACATTGTTTTAACGCAAAGCCCTG | |
| | CTATCATCGTGATTTCCGCCTCCCCAGGCGAGAAAGTCACT | |
| | ATGACCTGCTCGGCCAGTTCCTCTGTGTCTTACATGAATTG | |
| | GTATCAGCAGAAGTCCGGCACCTCTCCGAAAAGGTGGATC | |
| | TATGATACTTCCAAGCTGGCCTCTGGCGTGCCCGCACACTT | |
| | TCGGGGATCAGGGTCCGGGACAAGCTACAGCCTAACAATA | |
| | AGCGGTATGGAAGCTGAGGACGCAGCCACCTACTATTGTC | |
| | AGCAATGGAGCAGTAATCCCTTCACTTTTGGGTCTGGTACA | |
| | AAGCTCGAAATCAAGGAGCCCAAGTCTAGCGACAAAACCC | |
| | ACACAAGTCCACCTTCCCCTGCCCCAGAAGCTGCAGGCGG | |
| | ACCCTCCTCTATCGCCAGCTTCTTCTTCATCATCGGGCTGAT | |
| | TATCGGCCTGTTCCTGGTGCTGAGAGTGGGCATCCACCTGT | |
| | GCATCAAGCTGAAGCACACCAAGAAGCGGCAGATCTACAC | |
| | CGACATCGAGATGAACCGGCTGGGCAAATGATAA | |
| 644 | ATGGAGACTGACACACTGCTCTTATGGGTGCTTCTGTTGTG | HA-CD3.s.004.01- |
| | GGTCCCCGGCAGCACCGGGGATTACCCCTACGATGTGCCTG | IgG1ser-ICAM1 |
| | ACTATGCCGGAGGAAGTCAGGTGCAGCTGCAGCAGAGCGG | |
| | AGCCGAGCTGGCACGTCCAGGCGCCAGTGTGAAGATGTCC | |
| | TGTAAAGCGTCAGGGTACACCTTCACCAGATACACCATGCA | |
| | TTGGGTTAAACAGCGCCCCGGCCAGGGGCTTGAATGGATT | |
| | GGCTACATAAACCCTTCAAGGGGTTATACTAACTATAACCA | |
| | AAAGTTCAAAGACAAAGCTACACTGACAACTGACAAGTCA | |
| | AGTAGCACCGCTTACATGCAGCTTTCATCTCTGACCTCAGA | |
| | GGATAGTGCAGTCTATTATTGCGCGCGATATTACGACGATC | |
| | ACTACAGCCTCGATTACTGGGGACAAGGAACGACATTGAC | |
| | TGTATCGTCCGGGGGCGGTGGGTCTGGCGGAGGCGGGAGT | |
| | GGAGGAGGCGGTAGCGACATTGTTTTAACGCAAAGCCCTG | |
| | CTATCATCGTGATTTCCGCCTCCCCAGGCGAGAAAGTCACT | |
| | ATGACCTGCTCGGCCAGTTCCTCTGTGTCTTACATGAATTG | |
| | GTATCAGCAGAAGTCCGGCACCTCTCCGAAAAGGTGGATC | |
| | TATGATACTTCCAAGCTGGCCTCTGGCGTGCCCGCACACTT | |
| | TCGGGGATCAGGGTCCGGGACAAGCTACAGCCTAACAATA | |
| | AGCGGTATGGAAGCTGAGGACGCAGCCACCTACTATTGTC | |
| | AGCAATGGAGCAGTAATCCCTTCACTTTTGGGTCTGGTACA | |
| | AAGCTCGAAATCAAGGAGCCCAAGTCTAGCGACAAAACCC | |
| | ACACAAGTCCACCTTCCCCTGCCCCAGAAGCTGCAGGCGG | |
| | ACCCATAGTGATTATCACAGTTGTCGCCGCCGCTGTGATTA | |
| | TGGGAACCGCAGGCCTGAGCACTTATTTGTACAACCGGCA | |
| | GAGAAAGATCAAGAAATACAGGCTCTGATAA | |
| 645 | ATGGAGACTGACACACTGCTCTTATGGGTGCTTCTGTTGTG | HA-CD3.s.004.01- |
| | GGTCCCCGGCAGCACCGGGGATTACCCCTACGATGTGCCTG | IgG1ser-HLADRB |
| | ACTATGCCGGAGGAAGTCAGGTGCAGCTGCAGCAGAGCGG | |
| | AGCCGAGCTGGCACGTCCAGGCGCCAGTGTGAAGATGTCC | |
| | TGTAAAGCGTCAGGGTACACCTTCACCAGATACACCATGCA | |
| | TTGGGTTAAACAGCGCCCCGGCCAGGGGCTTGAATGGATT | |
| | GGCTACATAAACCCTTCAAGGGGTTATACTAACTATAACCA | |
| | AAAGTTCAAAGACAAAGCTACACTGACAACTGACAAGTCA | |
| | AGTAGCACCGCTTACATGCAGCTTTCATCTCTGACCTCAGA | |
| | GGATAGTGCAGTCTATTATTGCGCGCGATATTACGACGATC | |
| | ACTACAGCCTCGATTACTGGGGACAAGGAACGACATTGAC | |
| | TGTATCGTCCGGGGGCGGTGGGTCTGGCGGAGGCGGGAGT | |
| | GGAGGAGGCGGTAGCGACATTGTTTTAACGCAAAGCCCTG | |
| | CTATCATCGTGATTTCCGCCTCCCCAGGCGAGAAAGTCACT | |
| | ATGACCTGCTCGGCCAGTTCCTCTGTGTCTTACATGAATTG | |
| | GTATCAGCAGAAGTCCGGCACCTCTCCGAAAAGGTGGATC | |
| | TATGATACTTCCAAGCTGGCCTCTGGCGTGCCCGCACACTT | |
| | TCGGGGATCAGGGTCCGGGACAAGCTACAGCCTAACAATA | |
| | AGCGGTATGGAAGCTGAGGACGCAGCCACCTACTATTGTC | |
| | AGCAATGGAGCAGTAATCCCTTCACTTTTGGGTCTGGTACA | |
| | AAGCTCGAAATCAAGGAGCCCAAGTCTAGCGACAAAACCC | |
| | ACACAAGTCCACCTTCCCCTGCCCCAGAAGCTGCAGGCGG | |
| | ACCCATGCTGTCTGGTGTCGGAGGTTTTGTGCTCGGACTCT | |
| | TGTTTCTGGGGGCCGGCCTTTTCATCTACTTCAGGAACCAG | |
| | AAGGGCCACAGCGGGTTACAACCCACCGGCTTCCTGTCCTG | |
| | ATAA | |
| 646 | ATGGAGACTGACACACTGCTCTTATGGGTGCTTCTGTTGTG | HA-CD3.s.004.01- |
| | GGTCCCCGGCAGCACCGGGGATTACCCCTACGATGTGCCTG | IgG1ser-CD49d |
| | ACTATGCCGGAGGAAGTCAGGTGCAGCTGCAGCAGAGCGG | |
| | AGCCGAGCTGGCACGTCCAGGCGCCAGTGTGAAGATGTCC | |
| | TGTAAAGCGTCAGGGTACACCTTCACCAGATACACCATGCA | |
| | TTGGGTTAAACAGCGCCCCGGCCAGGGGCTTGAATGGATT | |
| | GGCTACATAAACCCTTCAAGGGGTTATACTAACTATAACCA | |
| | AAAGTTCAAAGACAAAGCTACACTGACAACTGACAAGTCA | |
| | AGTAGCACCGCTTACATGCAGCTTTCATCTCTGACCTCAGA | |
| | GGATAGTGCAGTCTATTATTGCGCGCGATATTACGACGATC | |
| | ACTACAGCCTCGATTACTGGGGACAAGGAACGACATTGAC | |
| | TGTATCGTCCGGGGGCGGTGGGTCTGGCGGAGGCGGGAGT | |
| | GGAGGAGGCGGTAGCGACATTGTTTTAACGCAAAGCCCTG | |
| | CTATCATCGTGATTTCCGCCTCCCCAGGCGAGAAAGTCACT | |
| | ATGACCTGCTCGGCCAGTTCCTCTGTGTCTTACATGAATTG | |
| | GTATCAGCAGAAGTCCGGCACCTCTCCGAAAAGGTGGATC | |
| | TATGATACTTCCAAGCTGGCCTCTGGCGTGCCCGCACACTT | |
| | TCGGGGATCAGGGTCCGGGACAAGCTACAGCCTAACAATA | |
| | AGCGGTATGGAAGCTGAGGACGCAGCCACCTACTATTGTC | |
| | AGCAATGGAGCAGTAATCCCTTCACTTTTGGGTCTGGTACA | |
| | AAGCTCGAAATCAAGGAGCCCAAGTCTAGCGACAAAACCC | |
| | ACACAAGTCCACCTTCCCCTGCCCCAGAAGCTGCAGGCGG | |
| | ACCCATAGTGATCATCAGCTCCAGTCTGCTGTTAGGGCTCA | |
| | TTGTTTTGCTTCTGATTTCTTATGTCATGTGGAAAGCCGGCT | |
| | TTTTCAAGAGGCAGTACAAGTCATGATAA | |
| 647 | ATGATGGACCAAGCCCGCAGCGCCTTTAGTAATCTGTTCGG | TFR1-IgG1ser- |
| | CGGGGAACCCTTATCCTATACCAGGTTCAGCTTGGCTAGAC | CD3.s.004.01-HA |
| | AGGTTGATGGCGACAACTCACACGTCGAAATGAAACTCGC | |
| | CGTGGACGAGGAGGAGAATGCAGATAATAACACAAAGGC | |
| | GAACGTGACGAAACCTAAGCGGTGTTCCGGATCTATTTGCT | |
| | ACGGTACTATAGCTGTAATTGTGTTCTTTCTTATCGGATTTA | |
| | TGATCGGCTACCTGGGGGAGCCCAAGTCTAGCGACAAAAC | |
| | CCACACAAGTCCACCTTCCCCTGCCCCAGAAGCTGCAGGCG | |
| | GACCCCAGGTGCAGCTGCAGCAGAGCGGAGCCGAGCTGGC | |
| | ACGTCCAGGCGCCAGTGTGAAGATGTCCTGTAAAGCGTCA | |
| | GGGTACACCTTCACCAGATACACCATGCATTGGGTTAAACA | |
| | GCGCCCCGGCCAGGGGCTTGAATGGATTGGCTACATAAAC | |
| | CCTTCAAGGGGTTATACTAACTATAACCAAAAGTTCAAAGA | |
| | CAAAGCTACACTGACAACTGACAAGTCAAGTAGCACCGCT | |
| | TACATGCAGCTTTCATCTCTGACCTCAGAGGATAGTGCAGT | |
| | CTATTATTGCGCGCGATATTACGACGATCACTACAGCCTCG | |
| | ATTACTGGGGACAAGGAACGACATTGACTGTATCGTCCGG | |
| | GGGCGGTGGGTCTGGCGGAGGCGGGAGTGGAGGAGGCGGT | |
| | AGCGACATTGTTTTAACGCAAAGCCCTGCTATCATCGTGAT | |
| | TTCCGCCTCCCCAGGCGAGAAAGTCACTATGACCTGCTCGG | |
| | CCAGTTCCTCTGTGTCTTACATGAATTGGTATCAGCAGAAG | |
| | TCCGGCACCTCTCCGAAAAGGTGGATCTATGATACTTCCAA | |
| | GCTGGCCTCTGGCGTGCCCGCACACTTTCGGGGATCAGGGT | |
| | CCGGGACAAGCTACAGCCTAACAATAAGCGGTATGGAAGC | |
| | TGAGGACGCAGCCACCTACTATTGTCAGCAATGGAGCAGT | |
| | AATCCCTTCACTTTTGGGTCTGGTACAAAGCTCGAAATCAA | |
| | GGGAGGAAGTTACCCCTACGATGTGCCTGACTATGCCTGAT | |
| | AA | |
| 648 | ATGGAGACTGACACACTGCTCTTATGGGTGCTTCTGTTGTG | HA-CD3.s.004.01- |
| | GGTCCCCGGCAGCACCGGGGATTACCCCTACGATGTGCCTG | EAAAKx3-PDGFR |
| | ACTATGCCGGAGGAAGTCAGGTGCAGCTGCAGCAGAGCGG | |
| | AGCCGAGCTGGCACGTCCAGGCGCCAGTGTGAAGATGTCC | |
| | TGTAAAGCGTCAGGGTACACCTTCACCAGATACACCATGCA | |
| | TTGGGTTAAACAGCGCCCCGGCCAGGGGCTTGAATGGATT | |
| | GGCTACATAAACCCTTCAAGGGGTTATACTAACTATAACCA | |
| | AAAGTTCAAAGACAAAGCTACACTGACAACTGACAAGTCA | |
| | AGTAGCACCGCTTACATGCAGCTTTCATCTCTGACCTCAGA | |
| | GGATAGTGCAGTCTATTATTGCGCGCGATATTACGACGATC | |
| | ACTACAGCCTCGATTACTGGGGACAAGGAACGACATTGAC | |
| | TGTATCGTCCGGGGGCGGTGGGTCTGGCGGAGGCGGGAGT | |
| | GGAGGAGGCGGTAGCGACATTGTTTTAACGCAAAGCCCTG | |
| | CTATCATCGTGATTTCCGCCTCCCCAGGCGAGAAAGTCACT | |
| | ATGACCTGCTCGGCCAGTTCCTCTGTGTCTTACATGAATTG | |
| | GTATCAGCAGAAGTCCGGCACCTCTCCGAAAAGGTGGATC | |
| | TATGATACTTCCAAGCTGGCCTCTGGCGTGCCCGCACACTT | |
| | TCGGGGATCAGGGTCCGGGACAAGCTACAGCCTAACAATA | |
| | AGCGGTATGGAAGCTGAGGACGCAGCCACCTACTATTGTC | |
| | AGCAATGGAGCAGTAATCCCTTCACTTTTGGGTCTGGTACA | |
| | AAGCTCGAAATCAAGGCGGAGGCTGCTGCTAAAGAGGCCG | |
| | CCGCCAAGGAAGCAGCAGCCAAGGCAAACGCTGTCGGCCA | |
| | GGACACCCAAGAGGTGATAGTGGTGCCACACAGCCTGCCC | |
| | TTCAAGGTAGTGGTCATCTCCGCAATCCTTGCCCTCGTTGTT | |
| | TTGACAATTATTTCTCTGATTATCTTAATCATGCTGTGGCAG | |
| | AAAAAGCCTAGGTGATAA | |
| 649 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | EAAAKx3-GPI |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGCGGAGGCTGCTGCTAAAGA | |
| | GGCCGCCGCCAAGGAAGCAGCAGCCAAGGCACAGCTTGAG | |
| | AACGGGGGCACATCTCTCTCCGAAAAGACTGTCCTGTTACT | |
| | GGTGACCCCTTTCTTGGCTGCCGCATGGAGCCTGCACCCCT | |
| | GATAA | |
| 650 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | EAAAKx3-VSVGan |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGCGGAGGCTGCTGCTAAAGA | |
| | GGCCGCCGCCAAGGAAGCAGCAGCCAAGGCATCCTCTATC | |
| | GCCAGCTTCTTCTTCATCATCGGGCTGATTATCGGCCTGTTC | |
| | CTGGTGCTGAGAGTGGGCATCCACCTGTGCATCAAGCTGAA | |
| | GCACACCAAGAAGCGGCAGATCTACACCGACATCGAGATG | |
| | AACCGGCTGGGCAAATGATAA | |
| 651 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | EAAAKx3-CD8aTm |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGCGGAGGCTGCTGCTAAAGA | |
| | GGCCGCCGCCAAGGAAGCAGCAGCCAAGGCAATTGCAAGT | |
| | CAACCACTTTCTTTACGACCCGAGGCCTGTAGGCCCGCTGC | |
| | TGGGGGTGCTGTTCATACCAGGGGCCTGGACTTCGCCTGCG | |
| | ACATATACATTTGGGCCCCTCTTGCAGGCACTTGTGGAGTG | |
| | CTGTTGTTATCCCTGGTCATCACCCTATATTGTAATCACCGT | |
| | AATCGCCGGCGAGTGTGTAAATGCCCTCGGCCAGTGGTTAT | |
| | CGCTAGCCAGCCGCTCAGCCTGAGGCCAGAAGCGTGCCGC | |
| | CCTGCAGCAGGAGGGGCCGTCCATACACGGGGCCTCGATT | |
| | TTGCCTGTGATATTTATATCTGGGCCCCCCTCGCGGGGACG | |
| | TGCGGTGTCCTGTTGCTGTCACTGGTGATCACACTCTACTG | |
| | CAACCACAGAAACAGGCGCAGAGTGTGTAAGTGCCCTAGA | |
| | CCCGTAGTGTGATAA | |
| 652 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | EAAAKx3-HLADRB |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGCGGAGGCTGCTGCTAAAGA | |
| | GGCCGCCGCCAAGGAAGCAGCAGCCAAGGCAATGCTGTCT | |
| | GGTGTCGGAGGTTTTGTGCTCGGACTCTTGTTTCTGGGGGC | |
| | CGGCCTTTTCATCTACTTCAGGAACCAGAAGGGCCACAGCG | |
| | GGTTACAACCCACCGGCTTCCTGTCCTGATAA | |
| 653 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | EAAAKx3-CD49d |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGCGGAGGCTGCTGCTAAAGA | |
| | GGCCGCCGCCAAGGAAGCAGCAGCCAAGGCAATAGTGATC | |
| | ATCAGCTCCAGTCTGCTGTTAGGGCTCATTGTTTTGCTTCTG | |
| | ATTTCTTATGTCATGTGGAAAGCCGGCTTTTTCAAGAGGCA | |
| | GTACAAGTCATGATAA | |
| 654 | GCCACCATGATGGACCAAGCCCGCAGCGCCTTTAGTAATCT | TFR1-EAAAKx3- |
| | GTTCGGCGGGGAACCCTTATCCTATACCAGGTTCAGCTTGG | CD3.s.004.01-HA |
| | CTAGACAGGTTGATGGCGACAACTCACACGTCGAAATGAA | |
| | ACTCGCCGTGGACGAGGAGGAGAATGCAGATAATAACACA | |
| | AAGGCGAACGTGACGAAACCTAAGCGGTGTTCCGGATCTA | |
| | TTTGCTACGGTACTATAGCTGTAATTGTGTTCTTTCTTATCG | |
| | GATTTATGATCGGCTACCTGGGGGCGGAGGCTGCTGCTAAA | |
| | GAGGCCGCCGCCAAGGAAGCAGCAGCCAAGGCACAGGTGC | |
| | AGCTGCAGCAGAGCGGAGCCGAGCTGGCACGTCCAGGCGC | |
| | CAGTGTGAAGATGTCCTGTAAAGCGTCAGGGTACACCTTCA | |
| | CCAGATACACCATGCATTGGGTTAAACAGCGCCCCGGCCA | |
| | GGGGCTTGAATGGATTGGCTACATAAACCCTTCAAGGGGTT | |
| | ATACTAACTATAACCAAAAGTTCAAAGACAAAGCTACACT | |
| | GACAACTGACAAGTCAAGTAGCACCGCTTACATGCAGCTTT | |
| | CATCTCTGACCTCAGAGGATAGTGCAGTCTATTATTGCGCG | |
| | CGATATTACGACGATCACTACAGCCTCGATTACTGGGGACA | |
| | AGGAACGACATTGACTGTATCGTCCGGGGGCGGTGGGTCT | |
| | GGCGGAGGCGGGAGTGGAGGAGGCGGTAGCGACATTGTTT | |
| | TAACGCAAAGCCCTGCTATCATCGTGATTTCCGCCTCCCCA | |
| | GGCGAGAAAGTCACTATGACCTGCTCGGCCAGTTCCTCTGT | |
| | GTCTTACATGAATTGGTATCAGCAGAAGTCCGGCACCTCTC | |
| | CGAAAAGGTGGATCTATGATACTTCCAAGCTGGCCTCTGGC | |
| | GTGCCCGCACACTTTCGGGGATCAGGGTCCGGGACAAGCT | |
| | ACAGCCTAACAATAAGCGGTATGGAAGCTGAGGACGCAGC | |
| | CACCTACTATTGTCAGCAATGGAGCAGTAATCCCTTCACTT | |
| | TTGGGTCTGGTACAAAGCTCGAAATCAAGGGAGGAAGTTA | |
| | CCCCTACGATGTGCCTGACTATGCCTGATAA | |
| 655 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | G4SP-DGFR |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGGGGGCGGTGGAAGCAACGCT | |
| | GTCGGCCAGGACACCCAAGAGGTGATAGTGGTGCCACACA | |
| | GCCTGCCCTTCAAGGTAGTGGTCATCTCCGCAATCCTTGCC | |
| | CTCGTTGTTTTGACAATTATTTCTCTGATTATCTTAATCATG | |
| | CTGTGGCAGAAAAAGCCTAGGTGATAA | |
| 656 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | AEAAAK-PDGFR |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGCGGAGGCTGCTGCTAAAAAC | |
| | GCTGTCGGCCAGGACACCCAAGAGGTGATAGTGGTGCCAC | |
| | ACAGCCTGCCCTTCAAGGTAGTGGTCATCTCCGCAATCCTT | |
| | GCCCTCGTTGTTTTGACAATTATTTCTCTGATTATCTTAATC | |
| | ATGCTGTGGCAGAAAAAGCCTAGGTGATAA | |
| 657 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | IgG3ser-PDGFR |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGAACTCAAAACACCCCTGGGC | |
| | GACACAACGCACACCTCACCTAGAAGCCCCGAACCCAAGT | |
| | CATCCGATACCCCACCGCCTAGTCCCCGGTCTCCTGAGCCA | |
| | AAATCCAGCGATACTCCTCCACCGAGTCCTAGGAGCCCTGA | |
| | GCCAAAGTCTTCGGACACTCCCCCACCATCCCCCAACGCTG | |
| | TCGGCCAGGACACCCAAGAGGTGATAGTGGTGCCACACAG | |
| | CCTGCCCTTCAAGGTAGTGGTCATCTCCGCAATCCTTGCCC | |
| | TCGTTGTTTTGACAATTATTTCTCTGATTATCTTAATCATGC | |
| | TGTGGCAGAAAAAGCCTAGGTGATAA | |
| 658 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | CD28-PDGFR |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGCCGCAGCTATTGAGGTGATG | |
| | TATCCTCCACCCTACCTCGACAATGAAAAAAGCAACGGGA | |
| | CCATAATCCACGTTAAGGGAAAACATTTGTGCCCTTCTCCA | |
| | CTGTTCCCGGGCCCCTCCAAGCCCAACGCTGTCGGCCAGGA | |
| | CACCCAAGAGGTGATAGTGGTGCCACACAGCCTGCCCTTCA | |
| | AGGTAGTGGTCATCTCCGCAATCCTTGCCCTCGTTGTTTTGA | |
| | CAATTATTTCTCTGATTATCTTAATCATGCTGTGGCAGAAA | |
| | AAGCCTAGGTGATAA | |
| 659 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | IgG3hng-PDGFR |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGAACTCAAGACCCCTCTGGGC | |
| | GACACTACACACACTTGTCCCAGATGCCCTGAACCTAAGTC | |
| | TTGTGATACGCCCCCACCCTGTCCCCGGTGCCCAGAGCCTA | |
| | AATCCTGTGACACACCCCCACCATGCCCTAGGTGCCCAGAG | |
| | CCGAAAAGCTGCGATACCCCTCCCCCGTGTCCAAACGCTGT | |
| | CGGCCAGGACACCCAAGAGGTGATAGTGGTGCCACACAGC | |
| | CTGCCCTTCAAGGTAGTGGTCATCTCCGCAATCCTTGCCCT | |
| | CGTTGTTTTGACAATTATTTCTCTGATTATCTTAATCATGCT | |
| | GTGGCAGAAAAAGCCTAGGTGATAA | |
| 660 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | IgG1hng-PDGFR |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGAACCTAAAAGCTGCGACAAG | |
| | ACACACACCTGCCCCCCATGTCCCGCCCCTGAGGCAGCTGG | |
| | CGGGCCAAACGCTGTCGGCCAGGACACCCAAGAGGTGATA | |
| | GTGGTGCCACACAGCCTGCCCTTCAAGGTAGTGGTCATCTC | |
| | CGCAATCCTTGCCCTCGTTGTTTTGACAATTATTTCTCTGAT | |
| | TATCTTAATCATGCTGTGGCAGAAAAAGCCTAGGTGATAA | |
| 661 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | IgG4g-PDGFR |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGAGTCCAAGTACGGACCTCCG | |
| | TGTCCCAGCTGCCCAAACGCTGTCGGCCAGGACACCCAAG | |
| | AGGTGATAGTGGTGCCACACAGCCTGCCCTTCAAGGTAGTG | |
| | GTCATCTCCGCAATCCTTGCCCTCGTTGTTTTGACAATTATT | |
| | TCTCTGATTATCTTAATCATGCTGTGGCAGAAAAAGCCTAG | |
| | GTGATAA | |
| 662 | GCCACCATGGAGACTGACACACTGCTCTTATGGGTGCTTCT | HA- |
| | GTTGTGGGTCCCCGGCAGCACCGGGGATTACCCCTACGATG | CD3.s.001.01G4SICAM1 |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGGGGGCGGTGGAAGCATAGTG | |
| | ATTATCACAGTTGTCGCCGCCGCTGTGATTATGGGAACCGC | |
| | AGGCCTGAGCACTTATTTGTACAACCGGCAGAGAAAGATC | |
| | AAGAAATACAGGCTCTGATAA | |
| 663 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | G4Sx3-ICAM1 |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGGGGGCGGCGGGTCCGGAGGT | |
| | GGAGGTTCTGGGGGCGGCGGAAGCATAGTGATTATCACAG | |
| | TTGTCGCCGCCGCTGTGATTATGGGAACCGCAGGCCTGAGC | |
| | ACTTATTTGTACAACCGGCAGAGAAAGATCAAGAAATACA | |
| | GGCTCTGATAA | |
| 664 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | AEAAAK-ICAM1 |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGCGGAGGCTGCTGCTAAAATA | |
| | GTGATTATCACAGTTGTCGCCGCCGCTGTGATTATGGGAAC | |
| | CGCAGGCCTGAGCACTTATTTGTACAACCGGCAGAGAAAG | |
| | ATCAAGAAATACAGGCTCTGATAA | |
| 665 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | CD8ah-ICAM1 |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGCGCTTTCCAACTCTATTATGT | |
| | ACTTCAGTCATTTCGTTCCCGTCTTTCTGCCTGCAAAGCCCA | |
| | CAACCACGCCTGCCCCAAGGCCACCCACACCCGCCCCTACT | |
| | ATCGCAAGCCAGCCTCTCAGCCTGCGGCCGGAGGCTTCAA | |
| | GACCAGCTGCCGGCGGAGCCGTGCACACCCGCGGGTTGGA | |
| | CATAGTGATTATCACAGTTGTCGCCGCCGCTGTGATTATGG | |
| | GAACCGCAGGCCTGAGCACTTATTTGTACAACCGGCAGAG | |
| | AAAGATCAAGAAATACAGGCTCTGATAA | |
| 666 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | IgG3ser-ICAM1 |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGAACTCAAAACACCCCTGGGC | |
| | GACACAACGCACACCTCACCTAGAAGCCCCGAACCCAAGT | |
| | CATCCGATACCCCACCGCCTAGTCCCCGGTCTCCTGAGCCA | |
| | AAATCCAGCGATACTCCTCCACCGAGTCCTAGGAGCCCTGA | |
| | GCCAAAGTCTTCGGACACTCCCCCACCATCCCCCATAGTGA | |
| | TTATCACAGTTGTCGCCGCCGCTGTGATTATGGGAACCGCA | |
| | GGCCTGAGCACTTATTTGTACAACCGGCAGAGAAAGATCA | |
| | AGAAATACAGGCTCTGATAA | |
| 667 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | IgG4-ser-ICAM1 |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGAGTCTAAGTACGGCCCGCCC | |
| | AGCCCTCCCTCCCCAATAGTGATTATCACAGTTGTCGCCGC | |
| | CGCTGTGATTATGGGAACCGCAGGCCTGAGCACTTATTTGT | |
| | ACAACCGGCAGAGAAAGATCAAGAAATACAGGCTCTGATA | |
| | A | |
| 668 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | CD28-ICAM1 |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGCCGCAGCTATTGAGGTGATG | |
| | TATCCTCCACCCTACCTCGACAATGAAAAAAGCAACGGGA | |
| | CCATAATCCACGTTAAGGGAAAACATTTGTGCCCTTCTCCA | |
| | CTGTTCCCGGGCCCCTCCAAGCCCATAGTGATTATCACAGT | |
| | TGTCGCCGCCGCTGTGATTATGGGAACCGCAGGCCTGAGCA | |
| | CTTATTTGTACAACCGGCAGAGAAAGATCAAGAAATACAG | |
| | GCTCTGATAA | |
| 669 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | IgG3hng-ICAM1 |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGAACTCAAGACCCCTCTGGGC | |
| | GACACTACACACACTTGTCCCAGATGCCCTGAACCTAAGTC | |
| | TTGTGATACGCCCCCACCCTGTCCCCGGTGCCCAGAGCCTA | |
| | AATCCTGTGACACACCCCCACCATGCCCTAGGTGCCCAGAG | |
| | CCGAAAAGCTGCGATACCCCTCCCCCGTGTCCAATAGTGAT | |
| | TATCACAGTTGTCGCCGCCGCTGTGATTATGGGAACCGCAG | |
| | GCCTGAGCACTTATTTGTACAACCGGCAGAGAAAGATCAA | |
| | GAAATACAGGCTCTGATAA | |
| 670 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | IgG1hng-ICAM1 |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGAACCTAAAAGCTGCGACAAG | |
| | ACACACACCTGCCCCCCATGTCCCGCCCCTGAGGCAGCTGG | |
| | CGGGCCAATAGTGATTATCACAGTTGTCGCCGCCGCTGTGA | |
| | TTATGGGAACCGCAGGCCTGAGCACTTATTTGTACAACCGG | |
| | CAGAGAAAGATCAAGAAATACAGGCTCTGATAA | |
| 671 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | IgG4g-ICAM1 |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGAGTCCAAGTACGGACCTCCG | |
| | TGTCCCAGCTGCCCAATAGTGATTATCACAGTTGTCGCCGC | |
| | CGCTGTGATTATGGGAACCGCAGGCCTGAGCACTTATTTGT | |
| | ACAACCGGCAGAGAAAGATCAAGAAATACAGGCTCTGATA | |
| | A | |
| 672 | GCCACCATGGAGACTGACACACTGCTCTTATGGGTGCTTCT | HA- |
| | GTTGTGGGTCCCCGGCAGCACCGGGGATTACCCCTACGATG | CD3.s.004.01G4SPDGFR |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGGGGGCGGTGGAAGCAACGC | |
| | TGTCGGCCAGGACACCCAAGAGGTGATAGTGGTGCCACAC | |
| | AGCCTGCCCTTCAAGGTAGTGGTCATCTCCGCAATCCTTGC | |
| | CCTCGTTGTTTTGACAATTATTTCTCTGATTATCTTAATCAT | |
| | GCTGTGGCAGAAAAAGCCTAGGTGATAA | |
| 673 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | G4Sx3-PDGFR |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGGGGGCGGCGGGTCCGGAGG | |
| | TGGAGGTTCTGGGGGCGGCGGAAGCAACGCTGTCGGCCAG | |
| | GACACCCAAGAGGTGATAGTGGTGCCACACAGCCTGCCCT | |
| | TCAAGGTAGTGGTCATCTCCGCAATCCTTGCCCTCGTTGTTT | |
| | TGACAATTATTTCTCTGATTATCTTAATCATGCTGTGGCAGA | |
| | AAAAGCCTAGGTGATAA | |
| 674 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | AEAAAK-PDGFR |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGCGGAGGCTGCTGCTAAAAA | |
| | CGCTGTCGGCCAGGACACCCAAGAGGTGATAGTGGTGCCA | |
| | CACAGCCTGCCCTTCAAGGTAGTGGTCATCTCCGCAATCCT | |
| | TGCCCTCGTTGTTTTGACAATTATTTCTCTGATTATCTTAAT | |
| | CATGCTGTGGCAGAAAAAGCCTAGGTGATAA | |
| 675 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | CD8ah-PDGFR |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGCGCTTTCCAACTCTATTATG | |
| | TACTTCAGTCATTTCGTTCCCGTCTTTCTGCCTGCAAAGCCC | |
| | ACAACCACGCCTGCCCCAAGGCCACCCACACCCGCCCCTAC | |
| | TATCGCAAGCCAGCCTCTCAGCCTGCGGCCGGAGGCTTCAA | |
| | GACCAGCTGCCGGCGGAGCCGTGCACACCCGCGGGTTGGA | |
| | CAACGCTGTCGGCCAGGACACCCAAGAGGTGATAGTGGTG | |
| | CCACACAGCCTGCCCTTCAAGGTAGTGGTCATCTCCGCAAT | |
| | CCTTGCCCTCGTTGTTTTGACAATTATTTCTCTGATTATCTT | |
| | AATCATGCTGTGGCAGAAAAAGCCTAGGTGATAA | |
| 676 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | IgG3ser-PDGFR |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGAACTCAAAACACCCCTGGG | |
| | CGACACAACGCACACCTCACCTAGAAGCCCCGAACCCAAG | |
| | TCATCCGATACCCCACCGCCTAGTCCCCGGTCTCCTGAGCC | |
| | AAAATCCAGCGATACTCCTCCACCGAGTCCTAGGAGCCCTG | |
| | AGCCAAAGTCTTCGGACACTCCCCCACCATCCCCCAACGCT | |
| | GTCGGCCAGGACACCCAAGAGGTGATAGTGGTGCCACACA | |
| | GCCTGCCCTTCAAGGTAGTGGTCATCTCCGCAATCCTTGCC | |
| | CTCGTTGTTTTGACAATTATTTCTCTGATTATCTTAATCATG | |
| | CTGTGGCAGAAAAAGCCTAGGTGATAA | |
| 677 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | IgG4-ser-PDGFR |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGAGTCTAAGTACGGCCCGCC | |
| | CAGCCCTCCCTCCCCAAACGCTGTCGGCCAGGACACCCAAG | |
| | AGGTGATAGTGGTGCCACACAGCCTGCCCTTCAAGGTAGTG | |
| | GTCATCTCCGCAATCCTTGCCCTCGTTGTTTTGACAATTATT | |
| | TCTCTGATTATCTTAATCATGCTGTGGCAGAAAAAGCCTAG | |
| | GTGATAA | |
| 678 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | CD28-PDGFR |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGCCGCAGCTATTGAGGTGAT | |
| | GTATCCTCCACCCTACCTCGACAATGAAAAAAGCAACGGG | |
| | ACCATAATCCACGTTAAGGGAAAACATTTGTGCCCTTCTCC | |
| | ACTGTTCCCGGGCCCCTCCAAGCCCAACGCTGTCGGCCAGG | |
| | ACACCCAAGAGGTGATAGTGGTGCCACACAGCCTGCCCTTC | |
| | AAGGTAGTGGTCATCTCCGCAATCCTTGCCCTCGTTGTTTTG | |
| | ACAATTATTTCTCTGATTATCTTAATCATGCTGTGGCAGAA | |
| | AAAGCCTAGGTGATAA | |
| 679 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | IgG3hng-PDGFR |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGAACTCAAGACCCCTCTGGG | |
| | CGACACTACACACACTTGTCCCAGATGCCCTGAACCTAAGT | |
| | CTTGTGATACGCCCCCACCCTGTCCCCGGTGCCCAGAGCCT | |
| | AAATCCTGTGACACACCCCCACCATGCCCTAGGTGCCCAGA | |
| | GCCGAAAAGCTGCGATACCCCTCCCCCGTGTCCAAACGCTG | |
| | TCGGCCAGGACACCCAAGAGGTGATAGTGGTGCCACACAG | |
| | CCTGCCCTTCAAGGTAGTGGTCATCTCCGCAATCCTTGCCC | |
| | TCGTTGTTTTGACAATTATTTCTCTGATTATCTTAATCATGC | |
| | TGTGGCAGAAAAAGCCTAGGTGATAA | |
| 680 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | IgG1hng-PDGFR |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGAACCTAAAAGCTGCGACAA | |
| | GACACACACCTGCCCCCCATGTCCCGCCCCTGAGGCAGCTG | |
| | GCGGGCCAAACGCTGTCGGCCAGGACACCCAAGAGGTGAT | |
| | AGTGGTGCCACACAGCCTGCCCTTCAAGGTAGTGGTCATCT | |
| | CCGCAATCCTTGCCCTCGTTGTTTTGACAATTATTTCTCTGA | |
| | TTATCTTAATCATGCTGTGGCAGAAAAAGCCTAGGTGATAA | |
| 681 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | IgG4g-PDGFR |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGAGTCCAAGTACGGACCTCC | |
| | GTGTCCCAGCTGCCCAAACGCTGTCGGCCAGGACACCCAA | |
| | GAGGTGATAGTGGTGCCACACAGCCTGCCCTTCAAGGTAGT | |
| | GGTCATCTCCGCAATCCTTGCCCTCGTTGTTTTGACAATTAT | |
| | TTCTCTGATTATCTTAATCATGCTGTGGCAGAAAAAGCCTA | |
| | GGTGATAA | |
| 682 | GCCACCATGGAGACTGACACACTGCTCTTATGGGTGCTTCT | HA- |
| | GTTGTGGGTCCCCGGCAGCACCGGGGATTACCCCTACGATG | CD3.s.004.01G4SICAM1 |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGGGGGCGGTGGAAGCATAGT | |
| | GATTATCACAGTTGTCGCCGCCGCTGTGATTATGGGAACCG | |
| | CAGGCCTGAGCACTTATTTGTACAACCGGCAGAGAAAGAT | |
| | CAAGAAATACAGGCTCTGATAA | |
| 683 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | G4Sx3-ICAM1 |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGGGGGCGGCGGGTCCGGAGG | |
| | TGGAGGTTCTGGGGGCGGCGGAAGCATAGTGATTATCACA | |
| | GTTGTCGCCGCCGCTGTGATTATGGGAACCGCAGGCCTGAG | |
| | CACTTATTTGTACAACCGGCAGAGAAAGATCAAGAAATAC | |
| | AGGCTCTGATAA | |
| 684 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | AEAAAK-ICAM1 |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGCGGAGGCTGCTGCTAAAAT | |
| | AGTGATTATCACAGTTGTCGCCGCCGCTGTGATTATGGGAA | |
| | CCGCAGGCCTGAGCACTTATTTGTACAACCGGCAGAGAAA | |
| | GATCAAGAAATACAGGCTCTGATAA | |
| 685 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | CD8αh-ICAM1 |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGCGCTTTCCAACTCTATTATG | |
| | TACTTCAGTCATTTCGTTCCCGTCTTTCTGCCTGCAAAGCCC | |
| | ACAACCACGCCTGCCCCAAGGCCACCCACACCCGCCCCTAC | |
| | TATCGCAAGCCAGCCTCTCAGCCTGCGGCCGGAGGCTTCAA | |
| | GACCAGCTGCCGGCGGAGCCGTGCACACCCGCGGGTTGGA | |
| | CATAGTGATTATCACAGTTGTCGCCGCCGCTGTGATTATGG | |
| | GAACCGCAGGCCTGAGCACTTATTTGTACAACCGGCAGAG | |
| | AAAGATCAAGAAATACAGGCTCTGATAA | |
| 686 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | IgG3ser-ICAM1 |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGAACTCAAAACACCCCTGGG | |
| | CGACACAACGCACACCTCACCTAGAAGCCCCGAACCCAAG | |
| | TCATCCGATACCCCACCGCCTAGTCCCCGGTCTCCTGAGCC | |
| | AAAATCCAGCGATACTCCTCCACCGAGTCCTAGGAGCCCTG | |
| | AGCCAAAGTCTTCGGACACTCCCCCACCATCCCCCATAGTG | |
| | ATTATCACAGTTGTCGCCGCCGCTGTGATTATGGGAACCGC | |
| | AGGCCTGAGCACTTATTTGTACAACCGGCAGAGAAAGATC | |
| | AAGAAATACAGGCTCTGATAA | |
| 687 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | CD28-ICAM1 |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGCCGCAGCTATTGAGGTGAT | |
| | GTATCCTCCACCCTACCTCGACAATGAAAAAAGCAACGGG | |
| | ACCATAATCCACGTTAAGGGAAAACATTTGTGCCCTTCTCC | |
| | ACTGTTCCCGGGCCCCTCCAAGCCCATAGTGATTATCACAG | |
| | TTGTCGCCGCCGCTGTGATTATGGGAACCGCAGGCCTGAGC | |
| | ACTTATTTGTACAACCGGCAGAGAAAGATCAAGAAATACA | |
| | GGCTCTGATAA | |
| 688 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | IgG3hng-ICAM1 |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGAACTCAAGACCCCTCTGGG | |
| | CGACACTACACACACTTGTCCCAGATGCCCTGAACCTAAGT | |
| | CTTGTGATACGCCCCCACCCTGTCCCCGGTGCCCAGAGCCT | |
| | AAATCCTGTGACACACCCCCACCATGCCCTAGGTGCCCAGA | |
| | GCCGAAAAGCTGCGATACCCCTCCCCCGTGTCCAATAGTGA | |
| | TTATCACAGTTGTCGCCGCCGCTGTGATTATGGGAACCGCA | |
| | GGCCTGAGCACTTATTTGTACAACCGGCAGAGAAAGATCA | |
| | AGAAATACAGGCTCTGATAA | |
| 689 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | IgG1hng-ICAM1 |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGAACCTAAAAGCTGCGACAA | |
| | GACACACACCTGCCCCCCATGTCCCGCCCCTGAGGCAGCTG | |
| | GCGGGCCAATAGTGATTATCACAGTTGTCGCCGCCGCTGTG | |
| | ATTATGGGAACCGCAGGCCTGAGCACTTATTTGTACAACCG | |
| | GCAGAGAAAGATCAAGAAATACAGGCTCTGATAA | |
| 690 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | IgG4g-ICAM1 |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGAGTCCAAGTACGGACCTCC | |
| | GTGTCCCAGCTGCCCAATAGTGATTATCACAGTTGTCGCCG | |
| | CCGCTGTGATTATGGGAACCGCAGGCCTGAGCACTTATTTG | |
| | TACAACCGGCAGAGAAAGATCAAGAAATACAGGCTCTGAT | |
| | AA | |
| 691 | GGCACCATGGAAACAGACACCCTCCTGCTGTGGGTCCTACT | RevisedHA- |
| | GTTATGGGTTCCTGGTAGCACAGGCTACCCCTACGATGTGC | UMTxOKT3 |
| | CTGACTATGCCGGAGGAAGTCAAGTGCAGCTGCAACAGAG | |
| | CGGCGCTGAGCTGGCGAGACCGGGCGCCTCTGTTAAGATG | |
| | AGTTGCAAAGCGTCTGGCTATACGTTTACGCGCTACACCAT | |
| | GCACTGGGTGAAACAGCGGCCTGGCCAGGGCCTAGAGTGG | |
| | ATTGGATACATCAACCCCTCAAGAGGGTACACTAACTACA | |
| | ATCAGAAGTTCAAGGATAAAGCTACCCTCACAACTGACAA | |
| | GTCATCTTCTACAGCCTATATGCAACTGTCCTCCCTCACCTC | |
| | CGAGGACTCAGCGGTGTACTACTGTGCAAGATATTATGACG | |
| | ATCATTATTGTCTGGATTACTGGGGCCAGGGGACCACCCTT | |
| | ACGGTTAGCTCCAGCGGCGGGGGAGGAAGCGGAGGCGGG | |
| | GGTAGCGGGGGAGGTGGTTCTCAGATTGTGCTCACACAAA | |
| | GCCCAGCCATCATGTCTGCTTCCCCGGGGGAAAAAGTCACC | |
| | ATGACCTGTAGCGCCTCCTCAAGTGTCAGTTATATGAATTG | |
| | GTATCAGCAGAAGTCGGGAACAAGCCCCAAGAGATGGATT | |
| | TACGATACCTCCAAACTGGCAAGCGGGGTGCCCGCCCACTT | |
| | CAGGGGTTCAGGCTCCGGCACTAGTTACTCACTTACCATCT | |
| | CCGGAATGGAGGCCGAAGATGCAGCAACTTACTATTGCCA | |
| | GCAGTGGAGTAGTAACCCTTTCACGTTTGGGTCTGGCACAA | |
| | AATTGGAAATCAATCGTTCAGATCCAACCACTACACCCGCT | |
| | CCTAGGCCCCCAACCCCGGCTCCAACTATAGCCTCGCAGCC | |
| | ACTTTCCCTCCGGCCCGAGGCCTGTCGCCCCGCTGCTGGGG | |
| | GAGCCGTCCACACACGCGGACTGGACTTTGCCTGCGACATC | |
| | TACATTTGGGCACCATTGGCAGGCACTTGCGGTGTACTGTT | |
| | GTTATCTCTTGTAATAACTCTGTATTGCAACCATCGAAATC | |
| | GGAGGAGGGTGTGTAAGTGCCCTCGACCTGTTGTGTGATAA | |
| 692 | GGCACCATGGAAACAGACACCCTCCTGCTGTGGGTCCTACT | RevisedOKT3 |
| | GTTATGGGTTCCTGGTAGCACAGGCCAAGTGCAGCTGCAAC | |
| | AGAGCGGCGCTGAGCTGGCGAGACCGGGCGCCTCTGTTAA | |
| | GATGAGTTGCAAAGCGTCTGGCTATACGTTTACGCGCTACA | |
| | CCATGCACTGGGTGAAACAGCGGCCTGGCCAGGGCCTAGA | |
| | GTGGATTGGATACATCAACCCCTCAAGAGGGTACACTAACT | |
| | ACAATCAGAAGTTCAAGGATAAAGCTACCCTCACAACTGA | |
| | CAAGTCATCTTCTACAGCCTATATGCAACTGTCCTCCCTCA | |
| | CCTCCGAGGACTCAGCGGTGTACTACTGTGCAAGATATTAT | |
| | GACGATCATTATTGTCTGGATTACTGGGGCCAGGGGACCAC | |
| | CCTTACGGTTAGCTCCAGCGGCGGGGGAGGAAGCGGAGGC | |
| | GGGGGTAGCGGGGGAGGTGGTTCTCAGATTGTGCTCACAC | |
| | AAAGCCCAGCCATCATGTCTGCTTCCCCGGGGGAAAAAGT | |
| | CACCATGACCTGTAGCGCCTCCTCAAGTGTCAGTTATATGA | |
| | ATTGGTATCAGCAGAAGTCGGGAACAAGCCCCAAGAGATG | |
| | GATTTACGATACCTCCAAACTGGCAAGCGGGGTGCCCGCCC | |
| | ACTTCAGGGGTTCAGGCTCCGGCACTAGTTACTCACTTACC | |
| | ATCTCCGGAATGGAGGCCGAAGATGCAGCAACTTACTATT | |
| | GCCAGCAGTGGAGTAGTAACCCTTTCACGTTTGGGTCTGGC | |
| | ACAAAATTGGAAATCAATCGTTCAGATCCAACCACTACACC | |
| | CGCTCCTAGGCCCCCAACCCCGGCTCCAACTATAGCCTCGC | |
| | AGCCACTTTCCCTCCGGCCCGAGGCCTGTCGCCCCGCTGCT | |
| | GGGGGAGCCGTCCACACACGCGGACTGGACTTTGCCTGCG | |
| | ACATCTACATTTGGGCACCATTGGCAGGCACTTGCGGTGTA | |
| | CTGTTGTTATCTCTTGTAATAACTCTGTATTGCAACCATCGA | |
| | AATCGGAGGAGGGTGTGTAAGTGCCCTCGACCTGTTGTGTG | |
| | ATAA | |
| 693 | GCCACCATGGAGACTGACACACTGCTCTTATGGGTGCTTCT | HA-ASGR1.s.017.02- |
| | GTTGTGGGTCCCCGGCAGCACCGGGGATTACCCCTACGATG | IgG1ser-PDGF |
| | TGCCTGACTATGCCGGAGGAAGTGAGGTTCAGCTCGTAGA | |
| | GTCCGGGGGCGGCCTCGTGCAACCTGGGGGCTCTCTTCGTC | |
| | TCTCTTGTGCCGCTTCAGGCTTTACCTTCTCCAGTTATAACA | |
| | TGAACTGGGTGCGCCAGGCCCCTGGCAAAGGGCTGGAATG | |
| | GGTGTCTTACATTAGCCGCTCTAGCAATACCAAGCACTATG | |
| | CAGACTCGGTGAAGGGTAGGTTTACCATAAGCAGGGACAA | |
| | CGCCAAAAATTCATTGTACTTACAAATGAACAGTTTACGAG | |
| | ACGAAGACACTGCTGTGTACTACTGCGCTCGGGACAGGAG | |
| | CGGATCTTACGGATATTTCTACTATTACGGCCTGGATGTCT | |
| | GGGGGCAGGGAACCACTGTCACAGTCAGTTCCGGAGGTGG | |
| | AGGCAGCGGAGGCGGGGGGAGTGGAGGGGGGGGTTCACA | |
| | GAGTGTCCTGACCCAGCCTCCATCTGCATCAGGCACGCCCG | |
| | GCCAACGGGTTACGATTTCATGTTCCGGGAGCAGTAGCAAT | |
| | ATCGGGTCCAATGCCGTGAACTGGTATCAGCAGCTGCCCGG | |
| | AACAGCGCCAAAACTGCTGATCTATTCAAATAATCAGAGA | |
| | CCCTCTGGCGTGCCGGACAGATTCAGCGGATCTAAGAGCG | |
| | GCACATCCGCATCCCTCGCCATCTCCGGACTGCAGTCGGAA | |
| | GATGAGGCTGATTACTATTGCGCGGCCTGGGATGATTCCCT | |
| | TAACGGTGTGGTTTTTGGCGGTGGTACAAAGTTGACTGTAC | |
| | TAGAGCCCAAGTCTAGCGACAAAACCCACACAAGTCCACC | |
| | TTCCCCTGCCCCAGAAGCTGCAGGCGGACCCAACGCTGTCG | |
| | GCCAGGACACCCAAGAGGTGATAGTGGTGCCACACAGCCT | |
| | GCCCTTCAAGGTAGTGGTCATCTCCGCAATCCTTGCCCTCG | |
| | TTGTTTTGACAATTATTTCTCTGATTATCTTAATCATGCTGT | |
| | GGCAGAAAAAGCCTAGGTGATAA | |
| 694 | GCCACCATGGAGACTGACACACTGCTCTTATGGGTGCTTCT | pCAGGS-SS- |
| | GTTGTGGGTCCCCGGCAGCACCGGGGATGGAGACGGAATG | GGlandingpad-RFP |
| | GTGTCTAAGGGCGAAGAGCTGATTAAGGAGAACATGCACA | |
| | TGAAGCTGTACATGGAGGGCACCGTGAACAACCACCACTT | |
| | CAAGTGCACATCCGAGGGCGAAGGCAAGCCCTACGAGGGC | |
| | ACCCAGACCATGAGAATCAAGGTGGTCGAGGGCGGCCCTC | |
| | TCCCCTTCGCCTTCGATATCCTGGCTACCAGCTTCATGTACG | |
| | GCAGCAGAACCTTCATCAACCACACCCAGGGCATCCCCGA | |
| | CTTCTTTAAGCAGTCCTTCCCTGAGGGCTTCACATGGGAGA | |
| | GAGTCACCACATACGAAGACGGGGGCGTGCTGACCGCTAC | |
| | CCAGGACACCAGCCTCCAGGACGGCTGCCTCATCTACAAC | |
| | GTCAAGATCAGAGGGGTGAACTTCCCATCCAACGGCCCTGT | |
| | GATGCAGAAGAAAACACTCGGCTGGGAGGCCAACACCGAG | |
| | ATGCTGTACCCCGCTGACGGCGGCCTGGAAGGCAGAAGCG | |
| | ACATGGCCCTGAAGCTCGTGGGCGGGGGCCACCTGATCTG | |
| | CAACTTCAAGACCACATACAGATCCAAGAAACCCGCTAAG | |
| | AACCTCAAGATGCCCGGCGTCTACTATGTGGACCACAGACT | |
| | GGAAAGAATCAAGGAGGCCGACAAAGAGACTTACGTCGAG | |
| | CAGCACGAGGTGGCTGTGGCCAGATACTGCGACCTCCCTA | |
| | GCAAACTGGGGCACAAACTTAATTGACGTCTCTCTATTGA | |
| 695 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | IgG4PDGFR-Dob |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGAGTCCAAGTACGGACCTCCG | |
| | TGTCCCAGCTGCCCATCTGCCTGCAGAGATCTGAAGAGATG | |
| | TCCCCGCGAACTACCGCCCACCCTGCTTGGCAATTCTTCCG | |
| | AAGAGGAAAGTCAGCTCGAAACTAACGTGACATACTGGGA | |
| | GGAGGAGCAGGAGTTTGAGGTTGTATCGACGCTGCGATTG | |
| | TGATAA | |
| 696 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | IgG4PDGFR-Dob2 |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGAGTCCAAGTACGGACCTCCG | |
| | TGTCCCAGCTGCCCATGATAA | |
| 697 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | EAAAKx3-ICAM1 |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGCGGAGGCTGCTGCTAAAGAG | |
| | GCCGCCGCCAAGGAAGCAGCAGCCAAGGCAATAGTGATTA | |
| | TCACAGTTGTCGCCGCCGCTGTGATTATGGGAACCGCAGGC | |
| | CTGAGCACTTATTTGTACAACCGGCAGAGAAAGATCAAGA | |
| | AATACAGGCTCTGATAA | |
| 698 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | IgG1ser-CD8aTm |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGAGCCCAAGTCTAGCGACAA | |
| | AACCCACACAAGTCCACCTTCCCCTGCCCCAGAAGCTGCAG | |
| | GCGGACCCATTGCAAGTCAACCACTTTCTTTACGACCCGAG | |
| | GCCTGTAGGCCCGCTGCTGGGGGTGCTGTTCATACCAGGGG | |
| | CCTGGACTTCGCCTGCGACATATACATTTGGGCCCCTCTTG | |
| | CAGGCACTTGTGGAGTGCTGTTGTTATCCCTGGTCATCACC | |
| | CTATATTGTAATCACCGTAATCGCCGGCGAGTGTGTAAATG | |
| | CCCTCGGCCAGTGGTTATCGCTAGCCAGCCGCTCAGCCTGA | |
| | GGCCAGAAGCGTGCCGCCCTGCAGCAGGAGGGGCCGTCCA | |
| | TACACGGGGCCTCGATTTTGCCTGTGATATTTATATCTGGG | |
| | CCCCCCTCGCGGGGACGTGCGGTGTCCTGTTGCTGTCACTG | |
| | GTGATCACACTCTACTGCAACCACAGAAACAGGCGCAGAG | |
| | TGTGTAAGTGCCCTAGACCCGTAGTGTGATAA | |
| 699 | GCCACCATGGAGACTGACACACTGCTCTTATGGGTGCTTCT | HA-CD3.s.004.01- |
| | GTTGTGGGTCCCCGGCAGCACCGGGGATTACCCCTACGATG | EAAAKx3-ICAM1 |
| | TGCCTGACTATGCCGGAGGAAGTCAGGTGCAGCTGCAGCA | |
| | GAGCGGAGCCGAGCTGGCACGTCCAGGCGCCAGTGTGAAG | |
| | ATGTCCTGTAAAGCGTCAGGGTACACCTTCACCAGATACAC | |
| | CATGCATTGGGTTAAACAGCGCCCCGGCCAGGGGCTTGAA | |
| | TGGATTGGCTACATAAACCCTTCAAGGGGTTATACTAACTA | |
| | TAACCAAAAGTTCAAAGACAAAGCTACACTGACAACTGAC | |
| | AAGTCAAGTAGCACCGCTTACATGCAGCTTTCATCTCTGAC | |
| | CTCAGAGGATAGTGCAGTCTATTATTGCGCGCGATATTACG | |
| | ACGATCACTACAGCCTCGATTACTGGGGACAAGGAACGAC | |
| | ATTGACTGTATCGTCCGGGGGCGGTGGGTCTGGCGGAGGC | |
| | GGGAGTGGAGGAGGCGGTAGCGACATTGTTTTAACGCAAA | |
| | GCCCTGCTATCATCGTGATTTCCGCCTCCCCAGGCGAGAAA | |
| | GTCACTATGACCTGCTCGGCCAGTTCCTCTGTGTCTTACATG | |
| | AATTGGTATCAGCAGAAGTCCGGCACCTCTCCGAAAAGGT | |
| | GGATCTATGATACTTCCAAGCTGGCCTCTGGCGTGCCCGCA | |
| | CACTTTCGGGGATCAGGGTCCGGGACAAGCTACAGCCTAA | |
| | CAATAAGCGGTATGGAAGCTGAGGACGCAGCCACCTACTA | |
| | TTGTCAGCAATGGAGCAGTAATCCCTTCACTTTTGGGTCTG | |
| | GTACAAAGCTCGAAATCAAGGCGGAGGCTGCTGCTAAAGA | |
| | GGCCGCCGCCAAGGAAGCAGCAGCCAAGGCAATAGTGATT | |
| | ATCACAGTTGTCGCCGCCGCTGTGATTATGGGAACCGCAGG | |
| | CCTGAGCACTTATTTGTACAACCGGCAGAGAAAGATCAAG | |
| | AAATACAGGCTCTGATAA | |
| 700 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | G4Sx3-PDGFR |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGGGGGCGGCGGGTCCGGAGGT | |
| | GGAGGTTCTGGGGGCGGCGGAAGCAACGCTGTCGGCCAGG | |
| | ACACCCAAGAGGTGATAGTGGTGCCACACAGCCTGCCCTTC | |
| | AAGGTAGTGGTCATCTCCGCAATCCTTGCCCTCGTTGTTTTG | |
| | ACAATTATTTCTCTGATTATCTTAATCATGCTGTGGCAGAA | |
| | AAAGCCTAGGTGATAA | |
| 701 | GCCACCATGGAGACTGACACACTGCTCTTATGGGTGCTTCT | HA-CD3.s.001.01- |
| | GTTGTGGGTCCCCGGCAGCACCGGGGATTACCCCTACGATG | CD8ah-PDGFR |
| | TGCCTGACTATGCCGGAGGAAGTGCGGCTCAGCCGGCATC | |
| | AGACATAAAACTCCAACAGTCAGGCGCTGAATTGGCTAGG | |
| | CCAGGGGCAAGCGTTAAGATGTCTTGTAAAACTTCCGGCTA | |
| | CACTTTTACGCGGTACACCATGCACTGGGTCAAACAGCGAC | |
| | CCGGCCAGGGGCTTGAGTGGATCGGCTACATTAACCCCTCG | |
| | AGAGGATACACCAACTACAATCAGAAGTTTAAGGACAAAG | |
| | CCACTCTAACAACCGACAAGTCTTCCTCAACCGCATATATG | |
| | CAGTTGTCCAGCCTGACATCTGAGGATTCCGCCGTGTATTA | |
| | CTGCGCACGTTATTATGACGATCATTATTGTCTTGACTACTG | |
| | GGGACAGGGCACTACACTGACAGTGTCCTCTGTTGAGGGT | |
| | GGAGGCGGCTCTGGGGGGGGAGGGAGCGGAGGGGGTGGG | |
| | AGCGTGGACGATATCCAGCTCACCCAGAGTCCTGCCATTAT | |
| | GTCTGCCAGTCCTGGAGAAAAGGTGACCATGACGTGCAGG | |
| | GCTAGCTCCTCAGTCAGCTACATGAACTGGTATCAACAGAA | |
| | GTCAGGCACTAGTCCCAAGCGCTGGATCTACGATACCTCCA | |
| | AGGTGGCCAGCGGAGTACCATATCGGTTCAGTGGCAGTGG | |
| | TAGCGGTACATCCTATTCGTTAACTATTTCAAGTATGGAAG | |
| | CTGAGGATGCCGCAACATACTACTGCCAACAATGGTCTAGC | |
| | AATCCTCTGACCTTCGGCGCCGGTACGAAACTGGAGCTGAA | |
| | AGCGGCCGCTAGAGGGTCCGCGCTTTCCAACTCTATTATGT | |
| | ACTTCAGTCATTTCGTTCCCGTCTTTCTGCCTGCAAAGCCCA | |
| | CAACCACGCCTGCCCCAAGGCCACCCACACCCGCCCCTACT | |
| | ATCGCAAGCCAGCCTCTCAGCCTGCGGCCGGAGGCTTCAA | |
| | GACCAGCTGCCGGCGGAGCCGTGCACACCCGCGGGTTGGA | |
| | CAACGCTGTCGGCCAGGACACCCAAGAGGTGATAGTGGTG | |
| | CCACACAGCCTGCCCTTCAAGGTAGTGGTCATCTCCGCAAT | |
| | CCTTGCCCTCGTTGTTTTGACAATTATTTCTCTGATTATCTT | |
| | AATCATGCTGTGGCAGAAAAAGCCTAGGTGATAA | |
| 702 | ATGGAGACTGACACACTGCTCTTATGGGTGCTTCTGTTGTG | HA-+C634: |
| | GGTCCCCGGCAGCACCGGGGATTACCCCTACGATGTGCCTG | C710CD3.s.004. |
| | ACTATGCCGGAGGAAGTCAGGTGCAGCTGCAGCAGAGCGG | 01-IgG4-ser-ICAM1 |
| | AGCCGAGCTGGCACGTCCAGGCGCCAGTGTGAAGATGTCC | |
| | TGTAAAGCGTCAGGGTACACCTTCACCAGATACACCATGCA | |
| | TTGGGTTAAACAGCGCCCCGGCCAGGGGCTTGAATGGATT | |
| | GGCTACATAAACCCTTCAAGGGGTTATACTAACTATAACCA | |
| | AAAGTTCAAAGACAAAGCTACACTGACAACTGACAAGTCA | |
| | AGTAGCACCGCTTACATGCAGCTTTCATCTCTGACCTCAGA | |
| | GGATAGTGCAGTCTATTATTGCGCGCGATATTACGACGATC | |
| | ACTACAGCCTCGATTACTGGGGACAAGGAACGACATTGAC | |
| | TGTATCGTCCGGGGGCGGTGGGTCTGGCGGAGGCGGGAGT | |
| | GGAGGAGGCGGTAGCGACATTGTTTTAACGCAAAGCCCTG | |
| | CTATCATCGTGATTTCCGCCTCCCCAGGCGAGAAAGTCACT | |
| | ATGACCTGCTCGGCCAGTTCCTCTGTGTCTTACATGAATTG | |
| | GTATCAGCAGAAGTCCGGCACCTCTCCGAAAAGGTGGATC | |
| | TATGATACTTCCAAGCTGGCCTCTGGCGTGCCCGCACACTT | |
| | TCGGGGATCAGGGTCCGGGACAAGCTACAGCCTAACAATA | |
| | AGCGGTATGGAAGCTGAGGACGCAGCCACCTACTATTGTC | |
| | AGCAATGGAGCAGTAATCCCTTCACTTTTGGGTCTGGTACA | |
| | AAGCTCGAAATCAAGGAGTCTAAGTACGGCCCGCCCAGCC | |
| | CTCCCTCCCCAATAGTGATTATCACAGTTGTCGCCGCCGCT | |
| | GTGATTATGGGAACCGCAGGCCTGAGCACTTATTTGTACAA | |
| | CCGGCAGAGAAAGATCAAGAAATACAGGCTCTGATAA | |
| 703 | METDTLLLWVLLLWVPGSTGDYPYDVPDYAGGSAAQPASDI | HA-CD3.s.001.01- |
| | KLQQSGAELARPGASVKMSCKTSGYTFTRYTMHWVKQRPGQ | IgG1ser-PDGFR |
| | GLEWIGYINPSRGYTNYNQKFKDKATLTTDKSSSTAYMQLSS | |
| | LTSEDSAVYYCARYYDDHYCLDYWGQGTTLTVSSVEGGGGS | |
| | GGGGSGGGGSVDDIQLTQSPAIMSASPGEKVTMTCRASSSVS | |
| | YMNWYQQKSGTSPKRWIYDTSKVASGVPYRFSGSGSGTSYSL | |
| | TISSMEAEDAATYYCQQWSSNPLTFGAGTKLELKAAARGSEP | |
| | KSSDKTHTSPPSPAPEAAGGPNAVGQDTQEVIVVPHSLPFKVV | |
| | VISAILALVVLTIISLIILIMLWQKKPR* | |
| 704 | METDTLLLWVLLLWVPGSTGDYPYDVPDYAGGSAAQPASDI | HA-CD3.s.001.01- |
| | KLQQSGAELARPGASVKMSCKTSGYTFTRYTMHWVKQRPGQ | IgG1ser-GPI |
| | GLEWIGYINPSRGYTNYNQKFKDKATLTTDKSSSTAYMQLSS | |
| | LTSEDSAVYYCARYYDDHYCLDYWGQGTTLTVSSVEGGGGS | |
| | GGGGSGGGGSVDDIQLTQSPAIMSASPGEKVTMTCRASSSVS | |
| | YMNWYQQKSGTSPKRWIYDTSKVASGVPYRFSGSGSGTSYSL | |
| | TISSMEAEDAATYYCQQWSSNPLTFGAGTKLELKAAARGSEP | |
| | KSSDKTHTSPPSPAPEAAGGPQLENGGTSLSEKTVLLLVTPFLA | |
| | AAWSLHP** | |
| 705 | METDTLLLWVLLLWVPGSTGDYPYDVPDYAGGSAAQPASDI | HA-CD3.s.001.01- |
| | KLQQSGAELARPGASVK | IgG1ser-VSVGan |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSEPKSSDKTH | |
| | TSPPSPAPEAAGGPSSIASFFFIIGLIIGLFLVLRVGIHLCIKLKHT | |
| | KKRQIYTDIEMNR LGK | |
| 706 | METDTLLLWVLLLWVPGSTGDYPYDVPDYAGGSAAQPASDI | HA-CD3.s.001.01- |
| | KLQQSGAELARPGASVKMSCKTSGYTFTRYTMHWVKQRPGQ | IgG1ser-CD8aTm |
| | GLEWIGYINPSRGYTNYNQKFKDKATLTTDKSSSTAYMQLSS | |
| | LTSEDSAVYYCARYYDDHYCLDYWGQGTTLTVSSVEGGGGS | |
| | GGGGSGGGGSVDDIQLTQSPAIMSASPGEKVTMTCRASSSVS | |
| | YMNWYQQKSGTSPKRWIYDTSKVASGVPYRFSGSGSGTSYSL | |
| | TISSMEAEDAATYYCQQWSSNPLTFGAGTKLELKAAARGSEP | |
| | KSSDKTHTSPPSPAPEAAGGPIASQPLSLRPEACRPAAGGAVH | |
| | TRGLDFACDIYIWAPLAGTCGVLLLSLVITLYCNHRNRRRVCK | |
| | CPRPVVIASQPLSLRPEACRPAAGGAVHTRGLDFACDIYIWAP | |
| | LAGTCGVLLLSLVITLYCNHRNRRRVCKCPRPVV* | |
| 707 | METDTLLLWVLLLWVPGSTGDYPYDVPDYAGGSAAQPASDI | HA-CD3.s.001.01- |
| | KLQQSGAELARPGASVK | IgG1ser-ICAM1 |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSEPKSSDKTH | |
| | TSPPSPAPEAAGGPIVIITVVAAAVIMGTAGLSTYLYNRQRKIK | |
| | KYRL** | |
| 708 | METDTLLLWVLLLWVPGSTGDYPYDVPDYAGGSAAQPASDI | HA-CD3.s.001.01- |
| | KLQQSGAELARPGASVKMS | IgG1ser-HLADRB |
| | CKTSGYTFTRYTMHWVKQRPGQGLEWIGYINPSRGYTNYNQ | |
| | KFKDKATLTTDKSSSTAYM | |
| | QLSSLTSEDSAVYYCARYYDDHYCLDYWGQGTTLTVSSVEG | |
| | GGGSGGGGSGGGGSVDDIQ | |
| | LTQSPAIMSASPGEKVTMTCRASSSVSYMNWYQQKSGTSPKR | |
| | WIYDTSKVASGVPYRFSG | |
| | SGSGTSYSLTISSMEAEDAATYYCQQWSSNPLTFGAGTKLELK | |
| | AAARGSEPKSSDKTHTS | |
| | PPSPAPEAAGGPMLSGVGGFVLGLLFLGAGLFIYFRNQKGHSG | |
| | LQPTGFLS** | |
| 709 | METDTLLLWVLLLWVPGSTGDYPYDVPDYAGGSAAQPASDI | HA-CD3.s.001.01- |
| | KLQQSGAELARPGASVK | IgG1ser-CD49d |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSEPKSSDKTH | |
| | TSPPSPAPEAAGGPIVIISSSLLLGLIVLLLISYVMWKAGFFKRQ | |
| | YKS** | |
| 710 | MMDQARSAFSNLFGGEPLSYTRFSLARQVDGDNSHVEMKLA | TFR1-IgG1ser- |
| | VDEEENADNNTKANVTKPK | CD3.s.001.01-HA |
| | RCSGSICYGTIAVIVFFLIGFMIGYLGEPKSSDKTHTSPPSPAPE | |
| | AAGGPAAQPASDIKL | |
| | QQSGAELARPGASVKMSCKTSGYTFTRYTMHWVKQRPGQGL | |
| | EWIGYINPSRGYTNYNQKF | |
| | KDKATLTTDKSSSTAYMQLSSLTSEDSAVYYCARYYDDHYCL | |
| | DYWGQGTTLTVSSVEGGG | |
| | GSGGGGSGGGGSVDDIQLTQSPAIMSASPGEKVTMTCRASSS | |
| | VSYMNWYQQKSGTSPKRW | |
| | IYDTSKVASGVPYRFSGSGSGTSYSLTISSMEAEDAATYYCQQ | |
| | WSSNPLTFGAGTKLELK AAARGSGGSYPYDVPDYA** | |
| 711 | METDTLLLWVLLLWVPGSTGDYPYDVPDYAGGSAAQPASDI | HA-CD3.s.001.01- |
| | KLQQSGAELARPGASVK | EAAAKx3-PDGFR |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSAEAAAKEAA | |
| | AKEAAAKANAVGQDTQEVIVVPHSLPFKVVVISAILALVVLTI | |
| | ISLIILIMLWQKKPR** | |
| 712 | METDTLLLWVLLLWVPGSTGDYPYDVPDYAGGSAAQPASDI | HA-CD3.s.001.01- |
| | KLQQSGAELARPGASVKMS | EAAAKx3-GPI |
| | CKTSGYTFTRYTMHWVKQRPGQGLEWIGYINPSRGYTNYNQ | |
| | KFKDKATLTTDKSSSTAYM | |
| | QLSSLTSEDSAVYYCARYYDDHYCLDYWGQGTTLTVSSVEG | |
| | GGGSGGGGSGGGGSVDDIQ | |
| | LTQSPAIMSASPGEKVTMTCRASSSVSYMNWYQQKSGTSPKR | |
| | WIYDTSKVASGVPYRFSG | |
| | SGSGTSYSLTISSMEAEDAATYYCQQWSSNPLTFGAGTKLELK | |
| | AAARGSAEAAAKEAAAK | |
| | EAAAKAQLENGGTSLSEKTVLLLVTPFLAAAWSLHP** | |
| 713 | METDTLLLWVLLLWVPGSTGDYPYDVPDYAGGSAAQPASDI | HA-CD3.s.001.01- |
| | KLQQSGAELARPGASVK | EAAAKx3-VSVGan |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSAEAAAKEAA | |
| | AKEAAAKASSIASFFFIIGLIIGLFLVLRVGIHLCIKLKHTKKRQI | |
| | YTDIEMNRLGK** | |
| 714 | METDTLLLWVLLLWVPGSTGDYPYDVPDYAGGSAAQPASDI | HA-CD3.s.001.01- |
| | KLQQSGAELARPGASVKMS | EAAAKx3-CD8aTm |
| | CKTSGYTFTRYTMHWVKQRPGQGLEWIGYINPSRGYTNYNQ | |
| | KFKDKATLTTDKSSSTAYM | |
| | QLSSLTSEDSAVYYCARYYDDHYCLDYWGQGTTLTVSSVEG | |
| | GGGSGGGGSGGGGSVDDIQ | |
| | LTQSPAIMSASPGEKVTMTCRASSSVSYMNWYQQKSGTSPKR | |
| | WIYDTSKVASGVPYRFSG | |
| | SGSGTSYSLTISSMEAEDAATYYCQQWSSNPLTFGAGTKLELK | |
| | AAARGSAEAAAKEAAAK | |
| | EAAAKAIASQPLSLRPEACRPAAGGAVHTRGLDFACDIYIWAP | |
| | LAGTCGVLLLSLVITLY | |
| | CNHRNRRRVCKCPRPVVIASQPLSLRPEACRPAAGGAVHTRG | |
| | LDFACDIYIWAPLAGTCG | |
| | VLLLSLVITLYCNHRNRRRVCKCPRPVV** | |
| 715 | METDTLLLWVLLLWVPGSTGDYPYDVPDYAGGSAAQPASDI | HA-CD3.s.001.01- |
| | KLQQSGAELARPGASVK | EAAAKx3-HLADRB |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSAEAAAKEAA | |
| | AKEAAAKAMLSGVGGFVLGLLFLGAGLFIYFRNQKGHSGLQP | |
| | TGFLS** | |
| 716 | METDTLLLWVLLLWVPGSTGDYPYDVPDYAGGSAAQPASDI | HA-CD3.s.001.01- |
| | KLQQSGAELARPGASVK | EAAAKx3-CD49d |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSAEAAAKEAA | |
| | AKEAAAKAIVIISSSLLLGLIVLLLISYVMWKAGFFKRQYKS** | |
| 717 | MMDQARSAFSNLFGGEPLSYTRFSLARQVDGDNSHVEMKLA | TFR1-EAAAKx3- |
| | VDEEENADNNTKANVTKPK | CD3.s.001.01-HA |
| | RCSGSICYGTIAVIVFFLIGFMIGYLGAEAAAKEAAAKEAAAK | |
| | AAAQPASDIKLQQSGAE | |
| | LARPGASVKMSCKTSGYTFTRYTMHWVKQRPGQGLEWIGYI | |
| | NPSRGYTNYNQKFKDKATL | |
| | TTDKSSSTAYMQLSSLTSEDSAVYYCARYYDDHYCLDYWGQ | |
| | GTTLTVSSVEGGGGSGGGG | |
| | SGGGGSVDDIQLTQSPAIMSASPGEKVTMTCRASSSVSYMNW | |
| | YQQKSGTSPKRWIYDTSK | |
| | VASGVPYRFSGSGSGTSYSLTISSMEAEDAATYYCQQWSSNPL | |
| | TFGAGTKLELKAAARGS GGSYPYDVPDYA** | |
| 718 | METDTLLLWVLLLWVPGSTGDYPYDVPDYAGGSQVQLQQSG | HA-CD3.s.004.01- |
| | AELARPGASVKMSCKAS | IgG1ser-PDGFR |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKEPKSSD | |
| | KTHTSPPSPAPEAAGGP | |
| | NAVGQDTQEVIVVPHSLPFKVVVISAILALVVLTIISLIILIMLW | |
| | QKKPR** | |
| 719 | METDTLLLWVLLLWVPGSTGDYPYDVPDYAGGSQVQLQQSG | HA-CD3.s.004.01- |
| | AELARPGASVKMSCKAS | IgG1ser-GPI |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKEPKSSD | |
| | KTHTSPPSPAPEAAGGP | |
| | QLENGGTSLSEKTVLLLVTPFLAAAWSLHP** | |
| 720 | METDTLLLWVLLLWVPGSTGDYPYDVPDYAGGSQVQLQQSG | HA-CD3.s.004.01- |
| | AELARPGASVKMSCKASGY | IgG1ser-VSVGan |
| | TFTRYTMHWVKQRPGQGLEWIGYINPSRGYTNYNQKFKDKA | |
| | TLTTDKSSSTAYMQLSSLT | |
| | SEDSAVYYCARYYDDHYSLDYWGQGTTLTVSSGGGGSGGGG | |
| | SGGGGSDIVLTQSPAIIVI | |
| | SASPGEKVTMTCSASSSVSYMNWYQQKSGTSPKRWIYDTSKL | |
| | ASGVPAHFRGSGSGTSYS | |
| | LTISGMEAEDAATYYCQQWSSNPFTFGSGTKLEIKEPKSSDKT | |
| | HTSPPSPAPEAAGGPSS | |
| | IASFFFIIGLIIGLFLVLRVGIHLCIKLKHTKKRQIYTDIEMNRLG | |
| | K** | |
| 721 | METDTLLLWVLLLWVPGSTGDYPYDVPDYAGGSQVQLQQSG | HA-CD3.s.004.01- |
| | AELARPGASVKMSCKAS | IgG1ser-ICAM1 |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKEPKSSD | |
| | KTHTSPPSPAPEAAGGP | |
| | IVIITVVAAAVIMGTAGLSTYLYNRQRKIKKYRL** | |
| 722 | METDTLLLWVLLLWVPGSTGDYPYDVPDYAGGSQVQLQQSG | HA-CD3.s.004.01- |
| | AELARPGASVKMSCKASGY | IgG1ser-HLADRB |
| | TFTRYTMHWVKQRPGQGLEWIGYINPSRGYTNYNQKFKDKA | |
| | TLTTDKSSSTAYMQLSSLT | |
| | SEDSAVYYCARYYDDHYSLDYWGQGTTLTVSSGGGGSGGGG | |
| | SGGGGSDIVLTQSPAIIVI | |
| | SASPGEKVTMTCSASSSVSYMNWYQQKSGTSPKRWIYDTSKL | |
| | ASGVPAHFRGSGSGTSYS | |
| | LTISGMEAEDAATYYCQQWSSNPFTFGSGTKLEIKEPKSSDKT | |
| | HTSPPSPAPEAAGGPML | |
| | SGVGGFVLGLLFLGAGLFIYFRNQKGHSGLQPTGFLS** | |
| 723 | METDTLLLWVLLLWVPGSTGDYPYDVPDYAGGSQVQLQQSG | HA-CD3.s.004.01- |
| | AELARPGASVKMSCKAS | IgG1ser-CD49d |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKEPKSSD | |
| | KTHTSPPSPAPEAAGGP | |
| | IVIISSSLLLGLIVLLLISYVMWKAGFFKRQYKS** | |
| 724 | MMDQARSAFSNLFGGEPLSYTRFSLARQVDGDNSHVEMKLA | TFR1-IgG1ser- |
| | VDEEENADNNTKANVTK | CD3.s.004.01-HA |
| | PKRCSGSICYGTIAVIVFFLIGFMIGYLGEPKSSDKTHTSPPSPA | |
| | PEAAGGPQVQLQQSG | |
| | AELARPGASVKMSCKASGYTFTRYTMHWVKQRPGQGLEWIG | |
| | YINPSRGYTNYNQKFKDKA | |
| | TLTTDKSSSTAYMQLSSLTSEDSAVYYCARYYDDHYSLDYW | |
| | GQGTTLTVSSGGGGSGGGG | |
| | SGGGGSDIVLTQSPAIIVISASPGEKVTMTCSASSSVSYMNWY | |
| | QQKSGTSPKRWIYDTSK | |
| | LASGVPAHFRGSGSGTSYSLTISGMEAEDAATYYCQQWSSNP | |
| | FTFGSGTKLEIKGGSYPY DVPDYA** | |
| 725 | METDTLLLWVLLLWVPGSTGDYPYDVPDYAGGSQVQLQQSG | HA-CD3.s.004.01- |
| | AELARPGASVKMSCKAS | EAAAKx3-PDGFR |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKAEAAA | |
| | KEAAAKEAAAKANAVGQD | |
| | TQEVIVVPHSLPFKVVVISAILALVVLTIISLIILIMLWQKKPR** | |
| 726 | ATMETDTLLLWVLLLWVPGSTGDYPYDVPDYAGGSQVQLQQ | HA-CD3.s.004.01- |
| | SGAELARPGASVKMSCKAS | EAAAKx3-GPI |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKAEAAA | |
| | KEAAAKEAAAKAQLENGG | |
| | TSLSEKTVLLLVTPFLAAAWSLHP** | |
| 727 | ATMETDTLLLWVLLLWVPGSTGDYPYDVPDYAGGSQVQLQQ | HA-CD3.s.004.01- |
| | SGAELARPGASVKMSCKAS | EAAAKx3-VSVGan |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKAEAAA | |
| | KEAAAKEAAAKASSIASF | |
| | FFIIGLIIGLFLVLRVGIHLCIKLKHTKKRQIYTDIEMNRLGK** | |
| 728 | ATMETDTLLLWVLLLWVPGSTGDYPYDVPDYAGGSQVQLQQ | HA-CD3.s.004.01- |
| | SGAELARPGASVKMSCKAS | EAAAKx3-CD8aTm |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKAEAAA | |
| | KEAAAKEAAAKAIASQPL | |
| | SLRPEACRPAAGGAVHTRGLDFACDIYIWAPLAGTCGVLLLSL | |
| | VITLYCNHRNRRRVCKC | |
| | PRPVVIASQPLSLRPEACRPAAGGAVHTRGLDFACDIYIWAPL | |
| | AGTCGVLLLSLVITLYC NHRNRRRVCKCPRPVV** | |
| 729 | ATMETDTLLLWVLLLWVPGSTGDYPYDVPDYAGGSQVQLQQ | HA-CD3.s.004.01- |
| | SGAELARPGASVKMSCKAS | EAAAKx3-HLADRB |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKAEAAA | |
| | KEAAAKEAAAKAMLSGVG | |
| | GFVLGLLFLGAGLFIYFRNQKGHSGLQPTGFLS** | |
| 730 | ATMETDTLLLWVLLLWVPGSTGDYPYDVPDYAGGSQVQLQQ | HA-CD3.s.004.01- |
| | SGAELARPGASVKMSCKAS | EAAAKx3-CD49d |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKAEAAA | |
| | KEAAAKEAAAKAIVIISS | |
| | SLLLGLIVLLLISYVMWKAGFFKRQYKS** | |
| 731 | ATMMDQARSAFSNLFGGEPLSYTRFSLARQVDGDNSHVEMK | TFR1-EAAAKx3- |
| | LAVDEEENADNNTKANVTK | CD3.s.004.01-HA |
| | PKRCSGSICYGTIAVIVFFLIGFMIGYLGAEAAAKEAAAKEAA | |
| | AKAQVQLQQSGAELARP | |
| | GASVKMSCKASGYTFTRYTMHWVKQRPGQGLEWIGYINPSR | |
| | GYTNYNQKFKDKATLTTDK | |
| | SSSTAYMQLSSLTSEDSAVYYCARYYDDHYSLDYWGQGTTL | |
| | TVSSGGGGSGGGGSGGGGS | |
| | DIVLTQSPAIIVISASPGEKVTMTCSASSSVSYMNWYQQKSGTS | |
| | PKRWIYDTSKLASGVP | |
| | AHFRGSGSGTSYSLTISGMEAEDAATYYCQQWSSNPFTFGSGT | |
| | KLEIKGGSYPYDVPDYA ** | |
| 732 | ATMETDTLLLWVLLLWVPGSTGDYPYDVPDYAGGSAAQPAS | HA-CD3.s.001.01- |
| | DIKLQQSGAELARPGASVK | G4SP-DGFR |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSGGGGSNAVG | |
| | QDTQEVIVVPHSLPFKVVVISAILALVVLTIISLIILIMLWQKKP | |
| | R** | |
| 733 | ATMETDTLLLWVLLLWVPGSTGDYPYDVPDYAGGSAAQPAS | HA-CD3.s.001.01- |
| | DIKLQQSGAELARPGASVK | AEAAAK-PDGFR |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSAEAAAKNAV | |
| | GQDTQEVIVVPHSLPFKVVVISAILALVVLTIISLIILIMLWQKK | |
| | PR** | |
| 734 | ATMETDTLLLWVLLLWVPGSTGDYPYDVPDYAGGSAAQPAS | HA-CD3.s.001.01- |
| | DIKLQQSGAELARPGASVK | IgG3ser-PDGFR |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSELKTPLGDT | |
| | THTSPRSPEPKSSDTPPPSPRSPEPKSSDTPPPSPRSPEPKSSDTP | |
| | PPSPNAVGQDTQEV | |
| | IVVPHSLPFKVVVISAILALVVLTIISLIILIMLWQKKPR** | |
| 735 | ATMETDTLLLWVLLLWVPGSTGDYPYDVPDYAGGSAAQPAS | HA-CD3.s.001.01- |
| | DIKLQQSGAELARPGASVK | CD28-PDGFR |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSAAAIEVMYP | |
| | PPYLDNEKSNGTIIHVKGKHLCPSPLFPGPSKPNAVGQDTQEVI | |
| | VVPHSLPFKVVVISAI LALVVLTIISLIILIMLWQKKPR** | |
| 736 | ATMETDTLLLWVLLLWVPGSTGDYPYDVPDYAGGSAAQPAS | HA-CD3.s.001.01- |
| | DIKLQQSGAELARPGASVK | IgG3hng-PDGFR |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSELKTPLGDT | |
| | THTCPRCPEPKSCDTPPPCPRCPEPKSCDTPPPCPRCPEPKSCDT | |
| | PPPCPNAVGQDTQEV | |
| | IVVPHSLPFKVVVISAILALVVLTIISLIILIMLWQKKPR** | |
| 737 | ATMETDTLLLWVLLLWVPGSTGDYPYDVPDYAGGSAAQPAS | HA-CD3.s.001.01- |
| | DIKLQQSGAELARPGASVK | IgG1hng-PDGFR |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSEPKSCDKTH | |
| | TCPPCPAPEAAGGPNAVGQDTQEVIVVPHSLPFKVVVISAILAL | |
| | VVLTIISLIILIMLWQ KKPR** | |
| 738 | ATMETDTLLLWVLLLWVPGSTGDYPYDVPDYAGGSAAQPAS | HA-CD3.s.001.01- |
| | DIKLQQSGAELARPGASVK | IgG4g-PDGFR |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSESKYGPPCP | |
| | SCPNAVGQDTQEVIVVPHSLPFKVVVISAILALVVLTIISLIILIM | |
| | LWQKKPR** | |
| 739 | ATMETDTLLLWVLLLWVPGSTGDYPYDVPDYAGGSAAQPAS | HA- |
| | DIKLQQSGAELARPGASVK | CD3.s.001.01G4SICAM1 |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSGGGGSIVII | |
| | TVVAAAVIMGTAGLSTYLYNRQRKIKKYRL** | |
| 740 | ATMETDTLLLWVLLLWVPGSTGDYPYDVPDYAGGSAAQPAS | HA-CD3.s.001.01- |
| | DIKLQQSGAELARPGASVK | G4Sx3-ICAM1 |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSGGGGSGGGG | |
| | SGGGGSIVIITVVAAAVIMGTAGLSTYLYNRQRKIKKYRL** | |
| 741 | ATMETDTLLLWVLLLWVPGSTGDYPYDVPDYAGGSAAQPAS | HA-CD3.s.001.01- |
| | DIKLQQSGAELARPGASVK | AEAAAK-ICAM1 |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSAEAAAKIVI | |
| | ITVVAAAVIMGTAGLSTYLYNRQRKIKKYRL** | |
| 742 | ATMETDTLLLWVLLLWVPGSTGDYPYDVPDYAGGSAAQPAS | HA-CD3.s.001.01- |
| | DIKLQQSGAELARPGASVK | CD8ah-ICAM1 |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSALSNSIMYF | |
| | SHFVPVFLPAKPTTTPAPRPPTPAPTIASQPLSLRPEASRPAAGG | |
| | AVHTRGLDIVIITVV | |
| | AAAVIMGTAGLSTYLYNRQRKIKKYRL** | |
| 743 | ATMETDTLLLWVLLLWVPGSTGDYPYDVPDYAGGSAAQPAS | HA-CD3.s.001.01- |
| | DIKLQQSGAELARPGASVK | IgG3ser-ICAM1 |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSELKTPLGDT | |
| | THTSPRSPEPKSSDTPPPSPRSPEPKSSDTPPPSPRSPEPKSSDTP | |
| | PPSPIVIITVVAAAVIMGTAGLSTYLYNRQRKIKKYRL** | |
| 744 | ATMETDTLLLWVLLLWVPGSTGDYPYDVPDYAGGSAAQPAS | HA-CD3.s.001.01- |
| | DIKLQQSGAELARPGASVK | IgG4-ser-ICAM1 |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSESKYGPPSP | |
| | PSPIVIITVVAAAVIMGTAGLSTYLYNRQRKIKKYRL** | |
| 745 | ATMETDTLLLWVLLLWVPGSTGDYPYDVPDYAGGSAAQPAS | HA-CD3.s.001.01- |
| | DIKLQQSGAELARPGASVK | CD28-ICAM1 |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSAAAIEVMYP | |
| | PPYLDNEKSNGTIIHVKGKHLCPSPLFPGPSKPIVIITVVAAAVI | |
| | MGTAGLSTYLYNRQR KIKKYRL** | |
| 746 | ATMETDTLLLWVLLLWVPGSTGDYPYDVPDYAGGSAAQPAS | HA-CD3.s.001.01- |
| | DIKLQQSGAELARPGASVK | IgG3hng-ICAM1 |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSELKTPLGDT | |
| | THTCPRCPEPKSCDTPPPCPRCPEPKSCDTPPPCPRCPEPKSCDT | |
| | PPPCPIVIITVVAAAVIMGTAGLSTYLYNRQRKIKKYRL** | |
| 747 | ATMETDTLLLWVLLLWVPGSTGDYPYDVPDYAGGSAAQPAS | HA-CD3.s.001.01- |
| | DIKLQQSGAELARPGASVK | IgG1hng-ICAM1 |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSEPKSCDKTH | |
| | TCPPCPAPEAAGGPIVIITVVAAAVIMGTAGLSTYLYNRQRKIK | |
| | KYRL** | |
| 748 | ATMETDTLLLWVLLLWVPGSTGDYPYDVPDYAGGSAAQPAS | HA-CD3.s.001.01- |
| | DIKLQQSGAELARPGASVK | IgG4g-ICAM1 |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSESKYGPPCP | |
| | SCPIVIITVVAAAVIMGTAGLSTYLYNRQRKIKKYRL** | |
| 749 | ATMETDTLLLWVLLLWVPGSTGDYPYDVPDYAGGSQVQLQQ | HA- |
| | SGAELARPGASVKMSCKAS | CD3.s.004.01G4SPDGFR |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKGGGGS | |
| | NAVGQDTQEVIVVPHSLP | |
| | FKVVVISAILALVVLTIISLIILIMLWQKKPR** | |
| 750 | ATMETDTLLLWVLLLWVPGSTGDYPYDVPDYAGGSQVQLQQ | HA-CD3.s.004.01- |
| | SGAELARPGASVKMSCKAS | G4Sx3-PDGFR |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKGGGGS | |
| | GGGGSGGGGSNAVGQDTQ | |
| | EVIVVPHSLPFKVVVISAILALVVLTIISLIILIMLWQKKPR** | |
| 751 | ATMETDTLLLWVLLLWVPGSTGDYPYDVPDYAGGSQVQLQQ | HA-CD3.s.004.01- |
| | SGAELARPGASVKMSCKAS | AEAAAK-PDGFR |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKAEAAA | |
| | KNAVGQDTQEVIVVPHSL | |
| | PFKVVVISAILALVVLTIISLIILIMLWQKKPR** | |
| 752 | ATMETDTLLLWVLLLWVPGSTGDYPYDVPDYAGGSQVQLQQ | HA-CD3.s.004.01- |
| | SGAELARPGASVKMSCKAS | CD8ah-PDGFR |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKALSNSI | |
| | MYFSHFVPVFLPAKPTT | |
| | TPAPRPPTPAPTIASQPLSLRPEASRPAAGGAVHTRGLDNAVG | |
| | QDTQEVIVVPHSLPFKV | |
| | VVISAILALVVLTIISLIILIMLWQKKPR** | |
| 753 | ATMETDTLLLWVLLLWVPGSTGDYPYDVPDYAGGSQVQLQQ | HA-CD3.s.004.01- |
| | SGAELARPGASVKMSCKAS | IgG3ser-PDGFR |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKELKTPL | |
| | GDTTHTSPRSPEPKSSD | |
| | TPPPSPRSPEPKSSDTPPPSPRSPEPKSSDTPPPSPNAVGQDTQE | |
| | VIVVPHSLPFKVVVISAILALVVLTIISLIILIMLWQKKPR** | |
| 754 | ATMETDTLLLWVLLLWVPGSTGDYPYDVPDYAGGSQVQLQQ | HA-CD3.s.004.01- |
| | SGAELARPGASVKMSCKAS | IgG4-ser-PDGFR |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKESKYGP | |
| | PSPPSPNAVGQDTQEVI | |
| | VVPHSLPFKVVVISAILALVVLTIISLIILIMLWQKKPR** | |
| 755 | ATMETDTLLLWVLLLWVPGSTGDYPYDVPDYAGGSQVQLQQ | HA-CD3.s.004.01- |
| | SGAELARPGASVKMSCKAS | CD28-PDGFR |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKAAAIEV | |
| | MYPPPYLDNEKSNGTII | |
| | HVKGKHLCPSPLFPGPSKPNAVGQDTQEVIVVPHSLPFKVVVI | |
| | SAILALVVLTIISLIILIMLWQKKPR** | |
| 756 | ATMETDTLLLWVLLLWVPGSTGDYPYDVPDYAGGSQVQLQQ | HA-CD3.s.004.01- |
| | SGAELARPGASVKMSCKAS | IgG3hng-PDGFR |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKELKTPL | |
| | GDTTHTCPRCPEPKSCD | |
| | TPPPCPRCPEPKSCDTPPPCPRCPEPKSCDTPPPCPNAVGQDTQ | |
| | EVIVVPHSLPFKVVVISAILALVVLTIISLIILIMLWQKKPR** | |
| 757 | ATMETDTLLLWVLLLWVPGSTGDYPYDVPDYAGGSQVQLQQ | HA-CD3.s.004.01- |
| | SGAELARPGASVKMSCKAS | IgG1hng-PDGFR |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKEPKSCD | |
| | KTHTCPPCPAPEAAGGP | |
| | NAVGQDTQEVIVVPHSLPFKVVVISAILALVVLTIISLIILIMLW | |
| | QKKPR** | |
| 758 | ATMETDTLLLWVLLLWVPGSTGDYPYDVPDYAGGSQVQLQQ | HA-CD3.s.004.01- |
| | SGAELARPGASVKMSCKAS | IgG4g-PDGFR |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKESKYGP | |
| | PCPSCPNAVGQDTQEVI | |
| | VVPHSLPFKVVVISAILALVVLTIISLIILIMLWQKKPR** | |
| 759 | ATMETDTLLLWVLLLWVPGSTGDYPYDVPDYAGGSQVQLQQ | HA- |
| | SGAELARPGASVKMSCKAS | CD3.s.004.01G4SICAM1 |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKGGGGSI | |
| | VIITVVAAAVIMGTAGLSTYLYNRQRKIKKYRL** | |
| 760 | ATMETDTLLLWVLLLWVPGSTGDYPYDVPDYAGGSQVQLQQ | HA-CD3.s.004.01- |
| | SGAELARPGASVKMSCKAS | G4Sx3-ICAM1 |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKGGGGS | |
| | GGGGSGGGGSIVIITVVA | |
| | AAVIMGTAGLSTYLYNRQRKIKKYRL** | |
| 761 | ATMETDTLLLWVLLLWVPGSTGDYPYDVPDYAGGSQVQLQQ | HA-CD3.s.004.01- |
| | SGAELARPGASVKMSCKAS | AEAAAK-ICAM1 |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKAEAAA | |
| | KIVIITVVAAAVIMGTAGLSTYLYNRQRKIKKYRL** | |
| 762 | ATMETDTLLLWVLLLWVPGSTGDYPYDVPDYAGGSQVQLQQ | HA-CD3.s.004.01- |
| | SGAELARPGASVKMSCKAS | CD8ah-ICAM1 |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKALSNSI | |
| | MYFSHFVPVFLPAKPTT | |
| | TPAPRPPTPAPTIASQPLSLRPEASRPAAGGAVHTRGLDIVIITV | |
| | VAAAVIMGTAGLSTYLYNRQRKIKKYRL** | |
| 763 | ATMETDTLLLWVLLLWVPGSTGDYPYDVPDYAGGSQVQLQQ | HA-CD3.s.004.01- |
| | SGAELARPGASVKMSCKAS | IgG3ser-ICAM1 |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKELKTPL | |
| | GDTTHTSPRSPEPKSSD | |
| | TPPPSPRSPEPKSSDTPPPSPRSPEPKSSDTPPPSPIVIITVVAAAV | |
| | IMGTAGLSTYLYNRQRKIKKYRL** | |
| 764 | ATMETDTLLLWVLLLWVPGSTGDYPYDVPDYAGGSQVQLQQ | HA-CD3.s.004.01- |
| | SGAELARPGASVKMSCKAS | CD28-ICAM1 |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKAAAIEV | |
| | MYPPPYLDNEKSNGTII | |
| | HVKGKHLCPSPLFPGPSKPIVIITVVAAAVIMGTAGLSTYLYNR | |
| | QRKIKKYRL** | |
| 765 | ATMETDTLLLWVLLLWVPGSTGDYPYDVPDYAGGSQVQLQQ | HA-CD3.s.004.01- |
| | SGAELARPGASVKMSCKAS | IgG3hng-ICAM1 |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKELKTPL | |
| | GDTTHTCPRCPEPKSCD | |
| | TPPPCPRCPEPKSCDTPPPCPRCPEPKSCDTPPPCPIVIITVVAAA | |
| | VIMGTAGLSTYLYNRQRKIKKYRL** | |
| 766 | ATMETDTLLLWVLLLWVPGSTGDYPYDVPDYAGGSQVQLQQ | HA-CD3.s.004.01- |
| | SGAELARPGASVKMSCKAS | IgG1hng-ICAM1 |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKEPKSCD | |
| | KTHTCPPCPAPEAAGGP | |
| | IVIITVVAAAVIMGTAGLSTYLYNRQRKIKKYRL** | |
| 767 | ATMETDTLLLWVLLLWVPGSTGDYPYDVPDYAGGSQVQLQQ | HA-CD3.s.004.01- |
| | SGAELARPGASVKMSCKAS | IgG4g-ICAM1 |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKESKYGP | |
| | PCPSCPIVIITVVAAAV IMGTAGLSTYLYNRQRKIKKYRL** | |
| 768 | GTMETDTLLLWVLLLWVPGSTGYPYDVPDYAGGSQVQLQQS | RevisedHA- |
| | GAELARPGASVKMSCKASG | UMTxOKT3 |
| | YTFTRYTMHWVKQRPGQGLEWIGYINPSRGYTNYNQKFKDK | |
| | ATLTTDKSSSTAYMQLSSL | |
| | TSEDSAVYYCARYYDDHYCLDYWGQGTTLTVSSSGGGGSGG | |
| | GGSGGGGSQIVLTQSPAIM | |
| | SASPGEKVTMTCSASSSVSYMNWYQQKSGTSPKRWIYDTSKL | |
| | ASGVPAHFRGSGSGTSYS | |
| | LTISGMEAEDAATYYCQQWSSNPFTFGSGTKLEINRSDPTTTP | |
| | APRPPTPAPTIASQPLS | |
| | LRPEACRPAAGGAVHTRGLDFACDIYIWAPLAGTCGVLLLSL | |
| | VITLYCNHRNRRRVCKCP RPVV** | |
| 769 | GTMETDTLLLWVLLLWVPGSTGQVQLQQSGAELARPGASVK | RevisedOKT3 |
| | MSCKASGYTFTRYTMHWVK | |
| | QRPGQGLEWIGYINPSRGYTNYNQKFKDKATLTTDKSSSTAY | |
| | MQLSSLTSEDSAVYYCAR | |
| | YYDDHYCLDYWGQGTTLTVSSSGGGGSGGGGSGGGGSQIVL | |
| | TQSPAIMSASPGEKVTMTC | |
| | SASSSVSYMNWYQQKSGTSPKRWIYDTSKLASGVPAHFRGSG | |
| | SGTSYSLTISGMEAEDAA | |
| | TYYCQQWSSNPFTFGSGTKLEINRSDPTTTPAPRPPTPAPTIAS | |
| | QPLSLRPEACRPAAGG | |
| | AVHTRGLDFACDIYIWAPLAGTCGVLLLSLVITLYCNHRNRRR | |
| | VCKCPRPVV** | |
| 770 | ATMETDTLLLWVLLLWVPGSTGDYPYDVPDYAGGSEVQLVE | HA-ASGR1.s.017.02- |
| | SGGGLVQPGGSLRLSCAAS | IgG1ser-PDGF |
| | GFTFSSYNMNWVRQAPGKGLEWVSYISRSSNTKHYADSVKG | |
| | RFTISRDNAKNSLYLQMNS | |
| | LRDEDTAVYYCARDRSGSYGYFYYYGLDVWGQGTTVTVSSG | |
| | GGGSGGGGSGGGGSQSVLT | |
| | QPPSASGTPGQRVTISCSGSSSNIGSNAVNWYQQLPGTAPKLLI | |
| | YSNNQRPSGVPDRESG | |
| | SKSGTSASLAISGLQSEDEADYYCAAWDDSLNGVVFGGGTKL | |
| | TVLEPKSSDKTHTSPPSP | |
| | APEAAGGPNAVGQDTQEVIVVPHSLPFKVVVISAILALVVLTII | |
| | SLIILIMLWQKKPR** | |
| 771 | ATMETDTLLLWVLLLWVPGSTGDGDGMVSKGEELIKENMH | pCAGGS-SS- |
| | MKLYMEGTVNNHHFKCTSEG | GGlandingpad-RFP |
| | EGKPYEGTQTMRIKVVEGGPLPFAFDILATSFMYGSRTFINHT | |
| | QGIPDFFKQSFPEGFTW | |
| | ERVTTYEDGGVLTATQDTSLQDGCLIYNVKIRGVNFPSNGPV | |
| | MQKKTLGWEANTEMLYPA | |
| | DGGLEGRSDMALKLVGGGHLICNFKTTYRSKKPAKNLKMPG | |
| | VYYVDHRLERIKEADKETY | |
| | VEQHEVAVARYCDLPSKLGHKLN* | |
| 772 | ATMETDTLLLWVLLLWVPGSTGDYPYDVPDYAGGSAAQPAS | HA-CD3.s.001.01- |
| | DIKLQQSGAELARPGASVK | IgG4PDGFR-Dob |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSESKYGPPCP | |
| | SCPSACRDLKRCPRELPPTLLGNSSEEESQLETNVTYWEEEQE | |
| | FEVVSTLRL** | |
| 773 | ATMETDTLLLWVLLLWVPGSTGDYPYDVPDYAGGSAAQPAS | HA-CD3.s.001.01- |
| | DIKLQQSGAELARPGASVK | IgG4PDGFR-Dob2 |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSESKYGPPCP SCP** | |
| 774 | ATMETDTLLLWVLLLWVPGSTGDYPYDVPDYAGGSAAQPAS | HA-CD3.s.001.01- |
| | DIKLQQSGAELARPGASVK | EAAAKx3-ICAM1 |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSAEAAAKEAA | |
| | AKEAAAKAIVIITVVAAAVIMGTAGLSTYLYNRQRKIKKYRL** | |
| 775 | ATMETDTLLLWVLLLWVPGSTGDYPYDVPDYAGGSQVQLQQ | HA-CD3.s.004.01- |
| | SGAELARPGASVKMSCKAS | IgG1ser-CD8aTm |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKEPKSSD | |
| | KTHTSPPSPAPEAAGGP | |
| | IASQPLSLRPEACRPAAGGAVHTRGLDFACDIYIWAPLAGTCG | |
| | VLLLSLVITLYCNHRNR | |
| | RRVCKCPRPVVIASQPLSLRPEACRPAAGGAVHTRGLDFACDI | |
| | YIWAPLAGTCGVLLLSL VITLYCNHRNRRRVCKCPRPVV** | |
| 776 | ATMETDTLLLWVLLLWVPGSTGDYPYDVPDYAGGSQVQLQQ | HA-CD3.s.004.01- |
| | SGAELARPGASVKMSCKAS | EAAAKx3-ICAM1 |
| | GYTFTRYTMHWVKQRPGQGLEWIGYINPSRGYTNYNQKFKD | |
| | KATLTTDKSSSTAYMQLSS | |
| | LTSEDSAVYYCARYYDDHYSLDYWGQGTTLTVSSGGGGSGG | |
| | GGSGGGGSDIVLTQSPAII | |
| | VISASPGEKVTMTCSASSSVSYMNWYQQKSGTSPKRWIYDTS | |
| | KLASGVPAHFRGSGSGTS | |
| | YSLTISGMEAEDAATYYCQQWSSNPFTFGSGTKLEIKAEAAA | |
| | KEAAAKEAAAKAIVIITV | |
| | VAAAVIMGTAGLSTYLYNRQRKIKKYRL** | |
| 777 | ATMETDTLLLWVLLLWVPGSTGDYPYDVPDYAGGSAAQPAS | HA-CD3.s.001.01- |
| | DIKLQQSGAELARPGASVK | G4Sx3-PDGFR |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSGGGGSGGGG | |
| | SGGGGSNAVGQDTQEVIVVPHSLPFKVVVISAILALVVLTIISLI | |
| | ILIMLWQKKPR** | |
| 778 | ATMETDTLLLWVLLLWVPGSTGDYPYDVPDYAGGSAAQPAS | HA-CD3.s.001.01- |
| | DIKLQQSGAELARPGASVK | CD8ah-PDGFR |
| | MSCKTSGYTFTRYTMHWVKQRPGQGLEWIGYINPSRGYTNY | |
| | NQKFKDKATLTTDKSSSTA | |
| | YMQLSSLTSEDSAVYYCARYYDDHYCLDYWGQGTTLTVSSV | |
| | EGGGGSGGGGSGGGGSVDD | |
| | IQLTQSPAIMSASPGEKVTMTCRASSSVSYMNWYQQKSGTSP | |
| | KRWIYDTSKVASGVPYRF | |
| | SGSGSGTSYSLTISSMEAEDAATYYCQQWSSNPLTFGAGTKLE | |
| | LKAAARGSALSNSIMYF | |
| | SHFVPVFLPAKPTTTPAPRPPTPAPTIASQPLSLRPEASRPAAGG | |
| | AVHTRGLDNAVGQDT | |
| | QEVIVVPHSLPFKVVVISAILALVVLTIISLIILIMLWQKKPR** | |
| 779 | METDTLLLWVLLLWVPGSTGDYPYDVPDYAGGSQVQLQQSG | HA-CD3.s.004.01- |
| | AELARPGASVKMSCKASGY | IgG4-ser-ICAM1 |
| | TFTRYTMHWVKQRPGQGLEWIGYINPSRGYTNYNQKFKDKA | |
| | TLTTDKSSSTAYMQLSSLT | |
| | SEDSAVYYCARYYDDHYSLDYWGQGTTLTVSSGGGGSGGGG | |
| | SGGGGSDIVLTQSPAIIVI | |
| | SASPGEKVTMTCSASSSVSYMNWYQQKSGTSPKRWIYDTSKL | |
| | ASGVPAHFRGSGSGTSYS | |
| | LTISGMEAEDAATYYCQQWSSNPFTFGSGTKLEIKESKYGPPS | |
| | PPSPIVIITVVAAAVIMGTAGLSTYLYNRQRKIKKYRL** | |
| 780 | (EAAAK)n | peptide linker, wherein |
| | | n is 1 to 10 |
| 781 | ALSNSIMYFSHFVPVFLPAKPTTTPAPRPPTPAPTIASQPLSLRP | CD8ah |
| | EASRPAAGGAVHTRGLD | |
| 782 | IVIITVVAAAVIMGTAGLSTYLYNRQRKIKKYRL | ICAM1 |
| 783 | LKTPLGDTTHTCPRCPEPKSCDTPPPCPRCPEPKSCDTPPPCPR | IgG3 hinge |
| | CPEPKSCDTPPPC | |
| 784 | PKSCDKTHTCPPCPAPEAAGG | IgG1 hinge |
| 785 | PNAVGQDTQEVIVVPHSLPFKVVVISAILALVVLTIISLIILIML | PDGFR |
| | WQKKPR | |
| 786 | SSIASFFFIIGLIIGLFLVLRVGIHLCIKLKHTKKRQIYTDIEMNR | VSVGan |
| | LGK | |
| 787 | IASQPLSLRPEACRPAAGGAVHTRGLDFACDIYIWAPLAGTCG | CD8aTm |
| | VLLLSLVITLYCNHRNRRRVCKCPRPVVIASQPLSLRPEACRPA | |
| | AGGAVHTRGLDFACDIYIWAPLAGTCGVLLLSLVITLYC | |
| 788 | MLSGVGGFVLGLLFLGAGLFIYFRNQKGHSGLQPTGFLS | HLADRB |
| 789 | IVIISSLLLGLIVLLLISYVMWKAGFFKRQYKS | CD49d |
| 790 | ESKYGPPCPSC | IgG4 |
| 791 | AAAIEVMYPPPYLDNEKSNGTIIHVKGKHLCPSPLFPGPSK | CD28 |
| 792 | ESKYGPPCPSCP | IgG4PDGFR |
| 793 | PQLENGGTSLSEKTVLLLVTPFLAAAWSLHP | GPI |
| 794 | MMDQARSAFSNLFGGEPLSYTRFSLARQVDGDNSHVEMKLA | Transferrin |
| | VDEEENADNNTKANVTKPKRCSGSICYGTIAVIVFFLIGFMIGY | |
| | LGEPKSSDKTHTSPPSPAPEAAGGP | |
| 795 | MGFTTKIIFLYNLVLVYAGFDDPRKAIELVQKRYGRPCDCSGG | BaEVTR |
| | QVSEPPSDRVSQVTCSGKTAYLMPDQRWKCKSI | |
| | PKDTSPSGPLQECPCNSYQSSVHSSCYTSYQQCRSGNKTYYTA | |
| | TLLKTQTGGTSDVQVLGSTNKLIQSPCNGIKG | |
| | QSICWSTTAPIHVSDGGGPLDTTRIKSVQRKLEEIHKALYPELQ | |
| | YHPLAIPKVRDNLMVDAQTLNILNATYNLLLMSN | |
| | TSLVDDCWLCLKLGPPTPLAIPNFLLSYVTRSSDNISCLIIPPLL | |
| | VQPMQFSNSSCLFSPSYNSTEEIDLGHVAFSNCT | |
| | SITNVTGPICAVNGSVFLCGNNMAYTYLPTNWTGLCVLATLL | |
| | PDIDIIPGDEPVPIPAIDHFIYRPKR | |
| | AIQFIPLLAGLGITAAFTTGATGLGVSVTQYTKLSNQLISDVQI | |
| | LSSTIQDLQDQVDSLAEVVLQNRRGLDLLTAEQGGI | |
| | CLALQEKCCFYVNKSGIVRDKIKTLQEELERRRKDLASNPLW | |
| | TGLQGLLPYLLPFLGPLLTLLLLLTIGPCIFNRLVQ | |
| | FVKDRISVVQALVLTQQYHQLKPLEYEP | |
| 796 | MGFTTKIIFLYNLVLVYAGFDDPRKAIELVQKRYGRPCDCSGG | BaEVRLess |
| | QVSEPPSDRVSQVTCSGKTAYLMPDQRWKCKSI | |
| | PKDTSPSGPLQECPCNSYQSSVHSSCYTSYQQCRSGNKTYYTA | |
| | TLLKTQTGGTSDVQVLGSTNKLIQSPCNGIKG | |
| | QSICWSTTAPIHVSDGGGPLDTTRIKSVQRKLEEIHKALYPELQ | |
| | YHPLAIPKVRDNLMVDAQTLNILNATYNLLLMSN | |
| | TSLVDDCWLCLKLGPPTPLAIPNFLLSYVTRSSDNISCLIIPPLL | |
| | VQPMQFSNSSCLFSPSYNSTEEIDLGHVAFSNCT | |
| | SITNVTGPICAVNGSVFLCGNNMAYTYLPTNWTGLCVLATLL | |
| | PDIDIIPGDEPVPIPAIDHFIYRPKRAIQFIPLLAGLGIT | |
| | AAFTTGATGLGVSVTQYTKLSNQLISDVQILSSTIQDLQDQVD | |
| | SLAEVVLQNRRGLDLLTAEQGGICLALQEKCCFYV | |
| | NKSGIVRDKIKTLQEELERRRKDLASNPLWTGLQGLLPYLLPF | |
| | LGPLLTLLLLLTIGPCIFNRLTAFINDKLNIIHAM | |
| 797 | MGFTTKIIFLYNLVLVYAGFDDPRKAIELVQKRYGRPCDCSGG | BaEVTR + protease |
| | QVSEPPSDRVSQVTCSGKTAYLMPDQRWKCKSI | mod |
| | PKDTSPSGPLQECPCNSYQSSVHSSCYTSYQQCRSGNKTYYTA | |
| | TLLKTQTGGTSDVQVLGSTNKLIQSPCNGIKG | |
| | QSICWSTTAPIHVSDGGGPLDTTRIKSVQRKLEEIHKALYPELQ | |
| | YHPLAIPKVRDNLMVDAQTLNILNATYNLLLMSN | |
| | TSLVDDCWLCLKLGPPTPLAIPNFLLSYVTRSSDNISCLIIPPLL | |
| | VQPMQFSNSSCLFSPSYNSTEEIDLGHVAFSNCT | |
| | SITNVTGPICAVNGSVFLCGNNMAYTYLPTNWTGLCVLATLL | |
| | PDIDIIPGDEPVPIPAIDHFIYRPKR | |
| | AIQFIPLLAGLGITAAFTTGATGLGVSVTQYTKLSNQLISDVQI | |
| | LSSTIQDLQDQVDSLAEVVLQNRRGLDLLTAEQGGI | |
| | CLALQEKCCFYVNKSGIVRDKIKTLQEELERRRKDLASNPLW | |
| | TGLQGLLPYLLPFLGPLLTLLLLLTIGPCIFNRLVQ | |
| | FVKDRISVSQNYPIVQQYHQLKPLEYEP | |
| 798 | GFDDPRKAIELVQKRYGRPCDCSGGQVSEPPSDRVSQVTCSG | BaEV |
| | KTAYLMPDQRWKCKSIPKDTSPSGPLQECPCNSYQSSVHSSCY | |
| | TSYQQCRSGNKTYYTATLLKTQTGGTSDVQVLGSTNKLIQSP | |
| | CNGIKGQSICWSTTAPIHVSDGGGPLDTTRIKSVQRKLEEIHKA | |
| | LYPELQYHPLAIPKVRDNLMVDAQTLNILNATYNLLLMSNTS | |
| | LVDDCWLCLKLGPPTPLAIPNFLLSYVTRSSDNISCLIIPPLLVQ | |
| | PMQFSNSSCLFSPSYNSTEEIDLGHVAFSNCTSITNVTGPICAV | |
| | NGSVFLCGNNMAYTYLPTNWTGLCVLATLLPDIDIIPGDEPVP | |
| | IPAIDHFIYRPKRAIQFIPLLAGLGITAAFTTGATGLGVSVTQYT | |
| | KLSNQLISDVQILSSTIQDLQDQVDSLAEVVLQNRRGLDLLTA | |
| | EQGGICLALQEKCCFYVNKSGIVRDKIKTLQEELERRRKDLAS | |
| | NPLWTGLQGLLPYLLPFLGPLLTLLLLLTIGPCIFNRLTAFIND | |
| | KLNIIHAMVLTQQYQVLRTDEEAQD | |
| 799 | Atggagactgacacactgctcttatgggtgcttctgttgtgggtccccggcagcaccggggattac | PLAS3310 -- HA- |
| | ccctacgatgtgcctgactatgccggaggaagtgaaatattggacgtgcccagcaccggaggag | RNlinker-F-IgG1ser- |
| | ggggttcgggagggggaggcagtggagggggcggcagtcctacagatctgcgtttcaccaatat | GPI |
| | cggtcctgacacaatgcgggtgacttgggcaccaccgccgagcattgatttgacaaattttctggtt | |
| | aggtacagccctgtgaagaacgaggaggatgtggcagagctgtcaatcagcccatccgacaatg | |
| | ccgttgtgcttaccaacctgctacccgggacagaatatgtggtctccgtgagtagtgtatacgagca | |
| | acacgagagcactccccttagaggtaggcagaagacaggtctggactcccccaccgggattgac | |
| | ttcagcgacattacggcgaactcttttactgtacattggattgcacctegcgcaactatcaccggcta | |
| | cagaattagacaccacccagaacatttcagcggcaggcccagggaggatcgcgtccctcactctc | |
| | gcaactctatcaccctcactaatctgacaccaggcactgaatacgtggtctccatagttgctctcaat | |
| | ggacgggaagagtcccccctgctgattggccagcagagcacggtgtccgatgtccctcgtgatct | |
| | ggaggtcgtcgctgctaccccaacctctctactcatatcatgggatgccccagctgtgacagtgcg | |
| | atattaccggattacctatggcgagacaggcggaaactccccggtccaggaatttactgtgcccgg | |
| | cagtaagtctactgccacaatttcgggacttaagcccggggttgactacaccatcacggtctacgc | |
| | cgtgacgggacggggcgactcccctgcctcttcaaagcctatctctattaactatagaacagaaatc | |
| | gacaaaccgagcgagcccaagtctagcgacaaaacccacacaagtccaccttcccctgccccag | |
| | aagctgcaggcggaccccagcttgagaacgggggcacatctctctccgaaaagactgtcctgtta | |
| | ctggtgacccctttcttggctgccgcatggagcctgcacccctgataa | |
| 800 | METDTLLLWVLLLWVPGSTGDYPYDVPDYAGGSEILDVPSTG | PLAS3310 -- HA- |
| | GGGSGGGGSGGGGSPTDL | RNlinker-F-IgG1ser- |
| | RFTNIGPDTMRVTWAPPPSIDLTNFLVRYSPVKNEEDVAELSIS | GPI |
| | PSDNAVVLTNLLPGTE | |
| | YVVSVSSVYEQHESTPLRGRQKTGLDSPTGIDESDITANSFTVH | |
| | WIAPRATITGYRIRHH | |
| | PEHFSGRPREDRVPHSRNSITLTNLTPGTEYVVSIVALNGREES | |
| | PLLIGQQSTVSDVPRD | |
| | LEVVAATPTSLLISWDAPAVTVRYYRITYGETGGNSPVQEFTV | |
| | PGSKSTATISGLKPGVD | |
| | YTITVYAVTGRGDSPASSKPISINYRTEIDKPSEPKSSDKTHTSP | |
| | PSPAPEAAGGPQLEN | |
| | GGTSLSEKTVLLLVTPFLAAAWSLHP | |
| 801 | QVQLVQSGAEVKKPGASVKVSCKASGYTFTSYYMHWVRQA | CD8 scFv |
| | PGQGLEWMGGFDPEDGETIYAQKFQGRVTMTRDTSTSTVYM | |
| | ELSSLRSEDTAVYYCARDQGWGMDVWGQGTTVTVSSGGGG | |
| | SGGGGSGGGGSDIQMTQSPSSLSASVGDRVTITCRASQSISSYL | |
| | NWYQQKPGKAPKLLIYAASSLQSGVPSRFSGSGSGTDFTLTISS | |
| | LQPEDFATYYCQQTYSTPYTFGQGTKLEIKR | |
| 802 | QVQLQQSGAELARPGASVKMSCKASGYTFTRYTMHWVKQR | CD3 scFV |
| | PGQGLEWIGYINPSRGYTNYNQKFKDKATLTTDKSSSTAYMQ | |
| | LSSLTSEDSAVYYCARYYDDHYSLDYWGQGTTLTVSSGGGG | |
| | SGGGGSGGGGSDIVLTQSPAIIVISASPGEKVTMTCSASSSVSY | |
| | MNWYQQKSGTSPKRWIYDTSKLASGVPAHFRGSGSGTSYSLT | |
| | ISGMEAEDAATYYCQQWSSNPFTFGSGTKLEIK | |
| 803 | ASTKGPSVFPLAPCSRSTSESTAALGCLVKDYFPEPVTVSWNS | IgG4 heavy chain seq |
| | GALTSGVHTFPAVLQSSGLYSLSSVVTVPSSSLGTKTYTCNVD | |
| | HKPSNTKVDKRVESKYGPPCPSCPAPEFLGGPSVFLFPPKPKD | |
| | TLMISRTPEVTCVVVDVSQEDPEVQFNWYVDGVEVHNAKTK | |
| | PREEQFNSTYRVVSVLTVLHQDWLNGKEYKCKVSNKGLPSSI | |
| | EKTISKAKGQPREPQVYTLPPSQEEMTKNQVSLTCLVKGFYPS | |
| | DIAVEWESNGQPENNYKTTPPVLDSDGSFFLYSRLTVDKSRW | |
| | QEGNVFSCSVMHEALHNHYTQKSLSLSLELQLEESCAEAQDG | |
| | ELDGLWTTITIFITLFLLSVCYSATVTFFKVKWIFSSVVDLKQTI | |
| | VPDYRNMIRQGA | |

1.-291. (canceled)

292. A lipid particle, comprising:
a. a Baboon Endogenous Virus (BaEV) fusogen or functional variant thereof, and
b. a co-display protein comprising a binding means for specifically interacting with a target molecule expressed on the surface of a target cell; and
wherein the protein in (a) and (b) are exposed on the outside of the lipid bilayer/embedded in the lipid bilayer.

293. The lipid particle of claim 292, wherein the co-display protein is an immune stimulating protein and/or is a transduction adjuvant.

294. The lipid particle of claim 292, wherein the co-display protein is selected from a glycoprotein, transmembrane protein, receptor, integrin, antibody or antigen-binding fragment, an engineered binding domain, a nanobody, a DARPin, an Aptamer, an Affimer, an Affibody, a Knottin, an Avimer, a Monobody, an Anticalin, a Fynomer, and a targeting peptide.

295. The lipid particle of claim 292, wherein the co-display protein increases specificity to a target cell selected from the group consisting of tumor-infiltrating lymphocytes, T cells, neoplastic or tumor cells, virus-infected cells, stem cells, central nervous system (CNS) cells, hematopoietic stem cells (HSCs), and liver cells.

296. The lipid particle of claim 292, wherein the co-display protein increases specificity to a target cell selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

297. The lipid particle of claim 292, wherein the co-display protein increases specificity to a target cell that is a hematopoietic stem cell.

298. The lipid particle of claim 292, wherein the target molecule is a protein, glycan, or lipid.

299. The lipid particle of claim 292, wherein the target molecule is selected from CD3, CD4, CD7, CD8, ASCT2, CD110, CD133, CD146, CD164, CD46, CD49f, CD90, EPCR, and ITGA3.

300. The lipid particle of claim 292, wherein the target molecule is CD133.

301. The lipid particle of claim 292, wherein the BaEV fusogen or functional variant thereof binds an ASCT-2 or ASCT-1 receptor.

302. The lipid particle of claim 292, wherein the BaEV fusogen or functional variant thereof comprises a protease site modification.

303. The lipid particle of claim 292, further comprising an exogenous agent for delivery to a target cell.

304. The lipid particle of claim 303, wherein the exogenous agent is a protein or a nucleic acid.

305. The lipid particle of claim 303, wherein the exogenous agent comprises a gRNA and Cas nuclease.

306. The lipid particle of claim 292, wherein the BaEV fusogen or functional variant thereof comprises the sequence set forth in SEQ ID NO: 795, or a sequence having at least 87%, 88%, 89%, 90%, 95%, 96%, 97%, 98%, or 99% sequence homology thereof.

307. The lipid particle of claim 292, wherein the BaEV fusogen or functional variant thereof lacks amino acids from the C-terminus of SEQ ID NO: 622.

308. The lipid particle of claim 307, wherein the amino acids are contiguous.

309. The lipid particle of claim 292, wherein the BaEV fusogen or functional variant thereof comprises an extracellular domain of a BaEV envelope glycoprotein.

310. A producer cell comprising (a) a nucleic acid encoding a retargeted attachment protein comprising a Baboon Endogenous Virus (BaEV) fusogen or functional variant thereof expressed on the surface of a target cell, and (b) a nucleic acid encoding a co-display protein.

311. The producer cell of claim 310, wherein the cell further comprises a viral nucleic acid(s).

312. The producer cell of claim 311, wherein the viral nucleic acid(s) are lentiviral nucleic acids.

313. The producer cell of claim 311, wherein the cell is a mammalian cell.

314. The producer cell of claim 311, wherein the producer cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

315. A method of making a lipid particle, comprising:
a. providing a producer cell of claim 311;
b. culturing the cell under conditions that allow for production of the lipid particle, and
c. separating, enriching, or purifying the lipid particle from the cell, thereby making the lipid particle.

316. A method of delivering an exogenous agent to a cell in a subject, the method comprising administering to the subject a lipid particle of claim 292.

317. The method of claim 316, wherein the exogenous agent is or encodes a therapeutic agent for treating a disease or condition in the subject.

318. The method of claim 316, wherein the exogenous agent comprises a gRNA and a Cas nuclease.

319. The method of claim 316, wherein the exogenous agent is for use in gene therapy to correct a genetic deficiency or replaces a deficient or missing gene in the subject.


SEQUENCE LISTING (803 sequences)

SEQ ID NO: 1 - protein, 602 aa, synthetic construct
  REGION 1..602: note = NiVG protein attachment glycoprotein (602 aa)
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 2 - protein, 557 aa, synthetic construct
  REGION 1..557: note = NiV-G backbone delCT
ILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 3 - protein, 557 aa, synthetic construct
  REGION 1..557: note = NiV-G backbone delCT and mutated
ILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 4 - protein, 45 aa, synthetic construct
  REGION 1..45: note = NivG CT_45_FL
MPAENKKVRFENTTSDKGKIPSKVIKSYYGTMDIKKINEGLLDSK

SEQ ID NO: 5 - protein, 602 aa, synthetic construct
  REGION 1..602: note = NiVG with NiVG CT_45_FL, mutated (E501 A, W504A, Q530A, E533A)
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 6 - protein, 5 aa, synthetic construct
  REGION 1..5: note = NivG_CT_05 cytoplasmic tail
MLDSK

SEQ ID NO: 7 - protein, 6 aa, synthetic construct
  REGION 1..6: note = NivG_CT_06 cytoplasmic tail
MLLDSK

SEQ ID NO: 8 - protein, 7 aa, synthetic construct
  REGION 1..7: note = NivG_CT_07 cytoplasmic tail
MGLLDSK

SEQ ID NO: 9 - protein, 8 aa, synthetic construct
  REGION 1..8: note = NivG_CT_08 cytoplasmic tail
MEGLLDSK

SEQ ID NO: 10 - protein, 9 aa, synthetic construct
  REGION 1..9: note = NivG_CT_09 cytoplasmic tail
MNEGLLDSK

SEQ ID NO: 11 - protein, 10 aa, synthetic construct
  REGION 1..10: note = NivG_CT_10 cytoplasmic tail
MINEGLLDSK

SEQ ID NO: 12 - protein, 11 aa, synthetic construct
  REGION 1..11: note = NivG_CT_11 cytoplasmic tail
MKINEGLLDSK

SEQ ID NO: 13 - protein, 13 aa, synthetic construct
  REGION 1..13: note = NivG_CT_13 cytoplasmic tail
MIKKINEGLLDSK

SEQ ID NO: 14 - protein, 14 aa, synthetic construct
  REGION 1..14: note = NivG_CT_14 cytoplasmic tail
MDIKKINEGLLDSK

SEQ ID NO: 15 - protein, 15 aa, synthetic construct
  REGION 1..15: note = NivG_CT_15 cytoplasmic tail
MMDIKKINEGLLDSK

SEQ ID NO: 16 - protein, 16 aa, synthetic construct
  REGION 1..16: note = NivG_CT_16 cytoplasmic tail
MTMDIKKINEGLLDSK

SEQ ID NO: 17 - protein, 17 aa, synthetic construct
  REGION 1..17: note = NivG_CT_17 cytoplasmic tail
MGTMDIKKINEGLLDSK

SEQ ID NO: 18 - protein, 18 aa, synthetic construct
  REGION 1..18: note = NivG_CT_18 cytoplasmic tail
MYGTMDIKKINEGLLDSK

SEQ ID NO: 19 - protein, 19 aa, synthetic construct
  REGION 1..19: note = NivG_CT_19 cytoplasmic tail
MYYGTMDIKKINEGLLDSK

SEQ ID NO: 20 - protein, 20 aa, synthetic construct
  REGION 1..20: note = NivG_CT_20 cytoplasmic tail
MSYYGTMDIKKINEGLLDSK

SEQ ID NO: 21 - protein, 21 aa, synthetic construct
  REGION 1..21: note = NivG_CT_21 cytoplasmic tail
MKSYYGTMDIKKINEGLLDSK

SEQ ID NO: 22 - protein, 22 aa, synthetic construct
  REGION 1..22: note = NivG_CT_22 cytoplasmic tail
MIKSYYGTMDIKKINEGLLDSK

SEQ ID NO: 23 - protein, 23 aa, synthetic construct
  REGION 1..23: note = NivG_CT_23 cytoplasmic tail
MVIKSYYGTMDIKKINEGLLDSK

SEQ ID NO: 24 - protein, 24 aa, synthetic construct
  REGION 1..24: note = NivG_CT_24 cytoplasmic tail
MKVIKSYYGTMDIKKINEGLLDSK

SEQ ID NO: 25 - protein, 25 aa, synthetic construct
  REGION 1..25: note = NivG_CT_25 cytoplasmic tail
MSKVIKSYYGTMDIKKINEGLLDSK

SEQ ID NO: 26 - protein, 30 aa, synthetic construct
  REGION 1..30: note = NivG_CT_30 cytoplasmic tail
MKGKNPSKVIKSYYGTMDIKKINEGLLDSK

SEQ ID NO: 27 - protein, 35 aa, synthetic construct
  REGION 1..35: note = NivG_CT_35 cytoplasmic tail
MNTTSDKGKNPSKVIKSYYGTMDIKKINEGLLDSK

SEQ ID NO: 28 - protein, 40 aa, synthetic construct
  REGION 1..40: note = NivG_CT_40 cytoplasmic tail
MKVRFENTTSDKGKNPSKVIKSYYGTMDIKKINEGLLDSK

SEQ ID NO: 29 - protein, 23 aa, synthetic construct
  REGION 1..23: note = NivG_CSUR38_CT_23 cytoplasmic tail
MIIKSYYGTMDIKKINEGLLDSK

SEQ ID NO: 30 - protein, 29 aa, synthetic construct
  REGION 1..29: note = NivG_CSUR38_CT_29 cytoplasmic tail
MGKIPSKIIKSYYGTMDIKKINEGLLDSK

SEQ ID NO: 31 - protein, 35 aa, synthetic construct
  REGION 1..35: note = NivG_CSUR38_CT_35 cytoplasmic tail
MNTTSDKGKIPSKIIKSYYGTMDIKKINEGLLDSK

SEQ ID NO: 32 - protein, 42 aa, synthetic construct
  REGION 1..42: note = NivG_CSUR38_CT_42 cytoplasmic tail
MSKKVRFENTTSDKGKIPSKIIKSYYGTMDIKKINEGLLDSK

SEQ ID NO: 33 - protein, 45 aa, synthetic construct
  REGION 1..45: note = NivG_CSUR38_CT_45_FL cytoplasmic tail
MPAESKKVRFENTTSDKGKIPSKIIKSYYGTMDIKKINEGLLDSK

SEQ ID NO: 34 - protein, 27 aa, synthetic construct
  REGION 1..27: note = NivG_VRI-0626_CT_27 cytoplasmic tail
MNPSKVIKSYYGTMDIKKINEGLLDSK

SEQ ID NO: 35 - protein, 31 aa, synthetic construct
  REGION 1..31: note = NivG_VRI-0626_CT_31 cytoplasmic tail
MDKGKNPSKVIKSYYGTMDIKKINEGLLDSK

SEQ ID NO: 36 - protein, 35 aa, synthetic construct; identical to SEQ ID NO: 27
  REGION 1..35: note = NivG_VRI-0626_CT_35

SEQ ID NO: 37 - protein, 40 aa, synthetic construct; identical to SEQ ID NO: 28
  REGION 1..40: note = NivG_VRI-0626_CT_40 cytoplasmic tail

SEQ ID NO: 38 - protein, 45 aa, synthetic construct
  REGION 1..45: note = NivG_VRI-0626_CT_45_FL cytoplasmic tail
MPAENKKVRFENTTSDKGKNPSKVIKSYYGTMDIKKINEGLLDSK

SEQ ID NO: 39 - protein, 12 aa, synthetic construct
  REGION 1..12: note = NivG_CT_12 cytoplasmic tail
MKKINEGLLDSK

SEQ ID NO: 40 - protein, 8 aa, synthetic construct
  REGION 1..8: note = HevG_CT_08
MDGLLDSK

SEQ ID NO: 41 - protein, 9 aa, synthetic construct
  REGION 1..9: note = HevG_CT_09
MNDGLLDSK

SEQ ID NO: 42 - protein, 10 aa, synthetic construct
  REGION 1..10: note = HevG_CT_10
MINDGLLDSK

SEQ ID NO: 43 - protein, 11 aa, synthetic construct
  REGION 1..11: note = HevG_CT_11
MKINDGLLDSK

SEQ ID NO: 44 - protein, 12 aa, synthetic construct
  REGION 1..12: note = HevG_CT_12
MKKINDGLLDSK

SEQ ID NO: 45 - protein, 13 aa, synthetic construct
  REGION 1..13: note = HevG_CT_13
MIKKINDGLLDSK

SEQ ID NO: 46 - protein, 14 aa, synthetic construct
  REGION 1..14: note = HevG_CT_14
MDIKKINDGLLDSK

SEQ ID NO: 47 - protein, 15 aa, synthetic construct
  REGION 1..15: note = HevG_CT_15
MMDIKKINDGLLDSK

SEQ ID NO: 48 - protein, 16 aa, synthetic construct
  REGION 1..16: note = HevG_CT_16
MTMDIKKINDGLLDSK

SEQ ID NO: 49 - protein, 17 aa, synthetic construct
  REGION 1..17: note = HevG_CT_17
MGTMDIKKINDGLLDSK

SEQ ID NO: 50 - protein, 18 aa, synthetic construct
  REGION 1..18: note = HevG_CT_18
MYGTMDIKKINDGLLDSK

SEQ ID NO: 51 - protein, 19 aa, synthetic construct
  REGION 1..19: note = HevG_CT_19
MYYGTMDIKKINDGLLDSK

SEQ ID NO: 52 - protein, 20 aa, synthetic construct
  REGION 1..20: note = HevG_CT_20
MNYYGTMDIKKINDGLLDSK

SEQ ID NO: 53 - protein, 21 aa, synthetic construct
  REGION 1..21: note = HevG_CT_21
MKNYYGTMDIKKINDGLLDSK

SEQ ID NO: 54 - protein, 22 aa, synthetic construct
  REGION 1..22: note = HevG_CT_22
MIKNYYGTMDIKKINDGLLDSK

SEQ ID NO: 55 - protein, 23 aa, synthetic construct
  REGION 1..23: note = HevG_CT_23
MVIKNYYGTMDIKKINDGLLDSK

SEQ ID NO: 56 - protein, 24 aa, synthetic construct
  REGION 1..24: note = HevG_CT_24
MKVIKNYYGTMDIKKINDGLLDSK

SEQ ID NO: 57 - protein, 45 aa, synthetic construct
  REGION 1..45: note = HevG_CT_45_FL
MMADSKLVSLNNNLSGKIKDQGKVIKNYYGTMDIKKINDGLLDSK

SEQ ID NO: 58 - protein, 6 aa, synthetic construct
  REGION 1..6: note = BatPV_G_CT_6
MQNDHY

SEQ ID NO: 59 - protein, 7 aa, synthetic construct
  REGION 1..7: note = BatPV_G_CT_7
MNQNDHY

SEQ ID NO: 60 - protein, 8 aa, synthetic construct
  REGION 1..8: note = BatPV_G_CT_8
MKNQNDHY

SEQ ID NO: 61 - protein, 9 aa, synthetic construct
  REGION 1..9: note = BatPV_G_CT_9
MQKNQNDHY

SEQ ID NO: 62 - protein, 10 aa, synthetic construct
  REGION 1..10: note = BatPV_G_CT_10
MKQKNQNDHY

SEQ ID NO: 63 - protein, 11 aa, synthetic construct
  REGION 1..11: note = BatPV_G_CT_11
MKKQKNQNDHY

SEQ ID NO: 64 - protein, 12 aa, synthetic construct
  REGION 1..12: note = BatPV_G_CT_12
MWKKQKNQNDHY

SEQ ID NO: 65 - protein, 13 aa, synthetic construct
  REGION 1..13: note = BatPV_G_CT_13
MNWKKQKNQNDHY

SEQ ID NO: 66 - protein, 14 aa, synthetic construct
  REGION 1..14: note = BatPV_G_CT_14
MRNWKKQKNQNDHY

SEQ ID NO: 67 - protein, 15 aa, synthetic construct
  REGION 1..15: note = BatPV_G_CT_15
MERNWKKQKNQNDHY

SEQ ID NO: 68 - protein, 16 aa, synthetic construct
  REGION 1..16: note = BatPV_G_CT_16
MSERNWKKQKNQNDHY

SEQ ID NO: 69 - protein, 17 aa, synthetic construct
  REGION 1..17: note = BatPV_G_CT_17
MHSERNWKKQKNQNDHY

SEQ ID NO: 70 - protein, 18 aa, synthetic construct
  REGION 1..18: note = BatPV_G_CT_18
MSHSERNWKKQKNQNDHY

SEQ ID NO: 71 - protein, 19 aa, synthetic construct
  REGION 1..19: note = BatPV_G_CT_19
MGSHSERNWKKQKNQNDHY

SEQ ID NO: 72 - protein, 20 aa, synthetic construct
  REGION 1..20: note = BatPV_G_CT_20
MLGSHSERNWKKQKNQNDHY

SEQ ID NO: 73 - protein, 21 aa, synthetic construct
  REGION 1..21: note = BatPV_G_CT_21
MGLGSHSERNWKKQKNQNDHY

SEQ ID NO: 74 - protein, 22 aa, synthetic construct
  REGION 1..22: note = BatPV_G_CT_22
MFGLGSHSERNWKKQKNQNDHY

SEQ ID NO: 75 - protein, 23 aa, synthetic construct
  REGION 1..23: note = BatPV_G_CT_23
MYFGLGSHSERNWKKQKNQNDHY

SEQ ID NO: 76 - protein, 24 aa, synthetic construct
  REGION 1..24: note = BatPV_G_CT_24
MGYFGLGSHSERNWKKQKNQNDHY

SEQ ID NO: 77 - protein, 29 aa, synthetic construct
  REGION 1..29: note = BatPV_G_CT_29
MKITKQGYFGLGSHSERNWKKQKNQNDHY

SEQ ID NO: 78 - protein, 59 aa, synthetic construct
  REGION 1..59: note = BatPV_G_CT_59_FL
MPQKTVEFINMNSPLERGVSTLSDKKTLNQSKITKQGYFGLGSHSERNWKKQKNQNDHY

SEQ ID NO: 79 - protein, 6 aa, synthetic construct
  REGION 1..6: note = CedarV_G_CT_6
MLHDIK

SEQ ID NO: 80 - protein, 7 aa, synthetic construct
  REGION 1..7: note = CedarV_G_CT_7
MSLHDIK

SEQ ID NO: 81 - protein, 8 aa, synthetic construct
  REGION 1..8: note = CedarV_G_CT_8
MESLHDIK

SEQ ID NO: 82 - protein, 9 aa, synthetic construct
  REGION 1..9: note = CedarV_G_CT_9
MNESLHDIK

SEQ ID NO: 83 - protein, 10 aa, synthetic construct
  REGION 1..10: note = CedarV_G_CT_10
MLNESLHDIK

SEQ ID NO: 84 - protein, 11 aa, synthetic construct
  REGION 1..11: note = CedarV_G_CT_11
MLLNESLHDIK

SEQ ID NO: 85 - protein, 12 aa, synthetic construct
  REGION 1..12: note = CedarV_G_CT_12
MNLLNESLHDIK

SEQ ID NO: 86 - protein, 13 aa, synthetic construct
  REGION 1..13: note = CedarV_G_CT_13
MSNLLNESLHDIK

SEQ ID NO: 87 - protein, 14 aa, synthetic construct
  REGION 1..14: note = CedarV_G_CT_14
MVSNLLNESLHDIK

SEQ ID NO: 88 - protein, 15 aa, synthetic construct
  REGION 1..15: note = CedarV_G_CT_15
MNVSNLLNESLHDIK

SEQ ID NO: 89 - protein, 16 aa, synthetic construct
  REGION 1..16: note = CedarV_G_CT_16
MYNVSNLLNESLHDIK

SEQ ID NO: 90 - protein, 17 aa, synthetic construct
  REGION 1..17: note = CedarV_G_CT_17
MNYNVSNLLNESLHDIK

SEQ ID NO: 91 - protein, 18 aa, synthetic construct
  REGION 1..18: note = CedarV_G_CT_18
MKNYNVSNLLNESLHDIK

SEQ ID NO: 92 - protein, 19 aa, synthetic construct
  REGION 1..19: note = CedarV_G_CT_19
MNKNYNVSNLLNESLHDIK

SEQ ID NO: 93 - protein, 20 aa, synthetic construct
  REGION 1..20: note = CedarV_G_CT_20
MKNKNYNVSNLLNESLHDIK

SEQ ID NO: 94 - protein, 21 aa, synthetic construct
  REGION 1..21: note = CedarV_G_CT_21
MVKNKNYNVSNLLNESLHDIK

SEQ ID NO: 95 - protein, 22 aa, synthetic construct
  REGION 1..22: note = CedarV_G_CT_22
MYVKNKNYNVSNLLNESLHDIK

SEQ ID NO: 96 - protein, 23 aa, synthetic construct
  REGION 1..23: note = CedarV_G_CT_23
MYYVKNKNYNVSNLLNESLHDIK

SEQ ID NO: 97 - protein, 24 aa, synthetic construct
  REGION 1..24: note = CedarV_G_CT_24
MSYYVKNKNYNVSNLLNESLHDIK

SEQ ID NO: 98 - protein, 30 aa, synthetic construct
  REGION 1..30: note = CedarV_G_CT_30
MQKDLNKSYYVKNKNYNVSNLLNESLHDIK

SEQ ID NO: 99 - protein, 68 aa, synthetic construct
  REGION 1..68: note = CedarV_G_CT_68_FL
MLSQLQKNYLDNSNQQGDKMNNPDKKLSVNFNPLELDKGQKDLNKSYYVKNKNYNVSNLLNESLHDIK

SEQ ID NO: 100 - protein, 6 aa, synthetic construct
  REGION 1..6: note = CDV-H_CT_06
MQGGRR

SEQ ID NO: 101 - protein, 7 aa, synthetic construct
  REGION 1..7: note = CDV-H_CT_07
MEQGGRR

SEQ ID NO: 102 - protein, 8 aa, synthetic construct
  REGION 1..8: note = CDV-H_CT_08
MEEQGGRR

SEQ ID NO: 103 - protein, 9 aa, synthetic construct
  REGION 1..9: note = CDV-H_CT_09
MTEEQGGRR

SEQ ID NO: 104 - protein, 10 aa, synthetic construct
  REGION 1..10: note = CDV-H_CT_10
MVTEEQGGRR

SEQ ID NO: 105 - protein, 11 aa, synthetic construct
  REGION 1..11: note = CDV-H_CT_11
MLVTEEQGGRR

SEQ ID NO: 106 - protein, 12 aa, synthetic construct
  REGION 1..12: note = CDV-H_CT_12
MSLVTEEQGGRR

SEQ ID NO: 107 - protein, 13 aa, synthetic construct
  REGION 1..13: note = CDV-H_CT_13
MLSLVTEEQGGRR

SEQ ID NO: 108 - protein, 14 aa, synthetic construct
  REGION 1..14: note = CDV-H_CT_14
MKLSLVTEEQGGRR

SEQ ID NO: 109 - protein, 15 aa, synthetic construct
  REGION 1..15: note = CDV-H_CT_15
MSKLSLVTEEQGGRR

SEQ ID NO: 110 - protein, 16 aa, synthetic construct
  REGION 1..16: note = CDV-H_CT_16
MSSKLSLVTEEQGGRR

SEQ ID NO: 111 - protein, 17 aa, synthetic construct
  REGION 1..17: note = CDV-H_CT_17
MNSSKLSLVTEEQGGRR

SEQ ID NO: 112 - protein, 18 aa, synthetic construct
  REGION 1..18: note = CDV-H_CT_18
MANSSKLSLVTEEQGGRR

SEQ ID NO: 113 - protein, 19 aa, synthetic construct
  REGION 1..19: note = CDV-H_CT_19
MRANSSKLSLVTEEQGGRR

SEQ ID NO: 114 - protein, 20 aa, synthetic construct
  REGION 1..20: note = CDV-H_CT_20
MARANSSKLSLVTEEQGGRR

SEQ ID NO: 115 - protein, 34 aa, synthetic construct
  REGION 1..34: note = CDV-H_CT_34_FL
MLSYQDKVGAFYKDNARANSSKLSLVTEEQGGRR

SEQ ID NO: 116 - protein, 6 aa, synthetic construct
  REGION 1..6: note = HPIV2-HN_CT_06
MRIIFR

SEQ ID NO: 117 - protein, 12 aa, synthetic construct
  REGION 1..12: note = HPIV2-HN_CT_12
MIPKRTCRIIFR

SEQ ID NO: 118 - protein, 22 aa, synthetic construct
  REGION 1..22: note = HPIV2-HN_CT_22_FL
MEDYSNLSLKSIPKRTCRIIFR

SEQ ID NO: 119 - protein, 6 aa, synthetic construct
  REGION 1..6: note = MvH_CT_06
MLMIDR

SEQ ID NO: 120 - protein, 7 aa, synthetic construct
  REGION 1..7: note = MvH_CT_07
MHLMIDR

SEQ ID NO: 121 - protein, 8 aa, synthetic construct
  REGION 1..8: note = MvH_CT_08
MEHLMIDR

SEQ ID NO: 122 - protein, 9 aa, synthetic construct
  REGION 1..9: note = MvH_CT_09
MREHLMIDR

SEQ ID NO: 123 - protein, 10 aa, synthetic construct
  REGION 1..10: note = MvH_CT_10
MNREHLMIDR

SEQ ID NO: 124 - protein, 11 aa, synthetic construct
  REGION 1..11: note = MvH_CT_11
MINREHLMIDR

SEQ ID NO: 125 - protein, 12 aa, synthetic construct
  REGION 1..12: note = MvH_CT_12
MVINREHLMIDR

SEQ ID NO: 126 - protein, 13 aa, synthetic construct
  REGION 1..13: note = MvH_CT_13
MIVINREHLMIDR

SEQ ID NO: 127 - protein, 14 aa, synthetic construct
  REGION 1..14: note = MvH_CT_14
MRIVINREHLMIDR

SEQ ID NO: 128 - protein, 15 aa, synthetic construct
  REGION 1..15: note = MvH_CT_15
MSRIVINREHLMIDR

SEQ ID NO: 129 - protein, 16 aa, synthetic construct
  REGION 1..16: note = MvH_CT_16
MGSRIVINREHLMIDR

SEQ ID NO: 130 - protein, 17 aa, synthetic construct
  REGION 1..17: note = MvH_CT_17
MKGSRIVINREHLMIDR

SEQ ID NO: 131 - protein, 18 aa, synthetic construct
  REGION 1..18: note = MvH_CT_18
MPKGSRIVINREHLMIDR

SEQ ID NO: 132 - protein, 19 aa, synthetic construct
  REGION 1..19: note = MvH_CT_19
MHPKGSRIVINREHLMIDR

SEQ ID NO: 133 - protein, 20 aa, synthetic construct
  REGION 1..20: note = MvH_CT_20
MPHPKGSRIVINREHLMIDR

SEQ ID NO: 134 - protein, 34 aa, synthetic construct
  REGION 1..34: note = MvH_CT_34_FL
MSPQRDRINAFYKDNPHPKGSRIVINREHLMIDR

SEQ ID NO: 135 - protein, 6 aa, synthetic construct
  REGION 1..6: note = NDV-HN_CT_06
MRLVFR

SEQ ID NO: 136 - protein, 7 aa, synthetic construct
  REGION 1..7: note = NDV-HN_CT_07
MWRLVFR

SEQ ID NO: 137 - protein, 8 aa, synthetic construct
  REGION 1..8: note = NDV-HN_CT_08
MTWRLVFR

SEQ ID NO: 138 - protein, 9 aa, synthetic construct
  REGION 1..9: note = NDV-HN_CT_09
MNTWRLVFR

SEQ ID NO: 139 - protein, 10 aa, synthetic construct
  REGION 1..10: note = NDV-HN_CT_10
MKNTWRLVFR

SEQ ID NO: 140 - protein, 11 aa, synthetic construct
  REGION 1..11: note = NDV-HN_CT_11
MAKNTWRLVFR

SEQ ID NO: 141 - protein, 12 aa, synthetic construct
  REGION 1..12: note = NDV-HN_CT_12
MEAKNTWRLVFR

SEQ ID NO: 142 - protein, 13 aa, synthetic construct
  REGION 1..13: note = NDV-HN_CT_13
MREAKNTWRLVFR

SEQ ID NO: 143 - protein, 14 aa, synthetic construct
  REGION 1..14: note = NDV-HN_CT_14
MEREAKNTWRLVFR

SEQ ID NO: 144 - protein, 15 aa, synthetic construct
  REGION 1..15: note = NDV-HN_CT_15
MDEREAKNTWRLVFR

SEQ ID NO: 145 - protein, 16 aa, synthetic construct
  REGION 1..16: note = NDV-HN_CT_16
MNDEREAKNTWRLVFR

SEQ ID NO: 146 - protein, 17 aa, synthetic construct
  REGION 1..17: note = NDV-HN_CT_17
MENDEREAKNTWRLVFR

SEQ ID NO: 147 - protein, 18 aa, synthetic construct
  REGION 1..18: note = NDV-HN_CT_18
MLENDEREAKNTWRLVFR

SEQ ID NO: 148 - protein, 19 aa, synthetic construct
  REGION 1..19: note = NDV-HN_CT_19
MALENDEREAKNTWRLVFR

SEQ ID NO: 149 - protein, 20 aa, synthetic construct
  REGION 1..20: note = NDV-HN_CT_20
MVALENDEREAKNTWRLVFR

SEQ ID NO: 150 - protein, 26 aa, synthetic construct
  REGION 1..26: note = NDV-HN_CT_26_FL
MERGVSQVALENDEREAKNTWRLVFR

SEQ ID NO: 151 - protein, 6 aa, synthetic construct
  REGION 1..6: note = SeV_CT_06
MERSGK

SEQ ID NO: 152 - protein, 7 aa, synthetic construct
  REGION 1..7: note = SeV_CT_07
MSERSGK

SEQ ID NO: 153 - protein, 8 aa, synthetic construct
  REGION 1..8: note = SeV_CT_08
MDSERSGK

SEQ ID NO: 154 - protein, 9 aa, synthetic construct
  REGION 1..9: note = SeV_CT_09
MSDSERSGK

SEQ ID NO: 155 - protein, 10 aa, synthetic construct
  REGION 1..10: note = SeV_CT_10
MVSDSERSGK

SEQ ID NO: 156 - protein, 11 aa, synthetic construct
  REGION 1..11: note = SeV_CT_11
MLVSDSERSGK

SEQ ID NO: 157 - protein, 12 aa, synthetic construct
  REGION 1..12: note = SeV_CT_12
MKLVSDSERSGK

SEQ ID NO: 158 - protein, 13 aa, synthetic construct
  REGION 1..13: note = SeV_CT_13
MTKLVSDSERSGK

SEQ ID NO: 159 - protein, 14 aa, synthetic construct
  REGION 1..14: note = SeV_CT_14
MTTKLVSDSERSGK

SEQ ID NO: 160 - protein, 15 aa, synthetic construct
  REGION 1..15: note = SeV_CT_15
MSTTKLVSDSERSGK

SEQ ID NO: 161 - protein, 16 aa, synthetic construct
  REGION 1..16: note = SeV_CT_16
MGSTTKLVSDSERSGK

SEQ ID NO: 162 - protein, 17 aa, synthetic construct
  REGION 1..17: note = SeV_CT_17
MGGSTTKLVSDSERSGK

SEQ ID NO: 163 - protein, 18 aa, synthetic construct
  REGION 1..18: note = SeV_CT_18
MPGGSTTKLVSDSERSGK

SEQ ID NO: 164 - protein, 19 aa, synthetic construct
  REGION 1..19: note = SeV_CT_19
MSPGGSTTKLVSDSERSGK

SEQ ID NO: 165 - protein, 20 aa, synthetic construct
  REGION 1..20: note = SeV_CT_20
MTSPGGSTTKLVSDSERSGK

SEQ ID NO: 166 - protein, 32 aa, synthetic construct
  REGION 1..32: note = SeV_CT_32_FL
MDGDRSKRDSYWSTSPGGSTTKLVSDSERSGK

SEQ ID NO: 167 - protein, 35 aa, synthetic construct
  REGION 1..35: note = BaEVwt_CT
MDQAEEDTRLVQYQQTLVMAHIINLKDNIFATLRN

SEQ ID NO: 168 - protein, 28 aa, synthetic construct
  REGION 1..28: note = BaEVRLess_CT
MAHIINLKDNIFATLRNKKINEGLLDSK

SEQ ID NO: 169 - protein, 17 aa, synthetic construct
  REGION 1..17: note = BaEVRLess_CT
MAHIINLKDNIFATLRN

SEQ ID NO: 170 - protein, 24 aa, synthetic construct
  REGION 1..24: note = Cocal_CT
MKRFRSMEIDNYIRKNNSGQYRYR

SEQ ID NO: 171 - protein, 35 aa, synthetic construct
  REGION 1..35: note = Cocal_CT
MKRFRSMEIDNYIRKNNSGQYRYRKKINEGLLDSK

SEQ ID NO: 172 - protein, 6 aa, synthetic construct
  REGION 1..6: note = EboV-GP_CT
MFVFKC

SEQ ID NO: 173 - protein, 17 aa, synthetic construct
  REGION 1..17: note = EboV-GP_CT
MFVFKCKKINEGLLDSK

SEQ ID NO: 174 - protein, 43 aa, synthetic construct
  REGION 1..43: note = GaLV_CT
MLNGENELAQYKQRLVLIKVASIRDNIFQVLKKKINEGLLDSK

SEQ ID NO: 175 - protein, 32 aa, synthetic construct
  REGION 1..32: note = GaLV_CT
MLNGENELAQYKQRLVLIKVASIRDNIFQVLK

SEQ ID NO: 176 - protein, 28 aa, synthetic construct
  REGION 1..28: note = GaLV-RLess_CT
MLIKVASIRDNIFQVLKKKINEGLLDSK

SEQ ID NO: 177 - protein, 17 aa, synthetic construct
  REGION 1..17: note = GaLV-RLess_CT
MLIKVASIRDNIFQVLK

SEQ ID NO: 178 - protein, 40 aa, synthetic construct
  REGION 1..40: note = LCMV_GP_CT
MRRKWVTKVGPVKFAGCSCIGKNTLRHPKPCSGGKIHRHT

SEQ ID NO: 179 - protein, 44 aa, synthetic construct
  REGION 1..44: note = MLV-A_CT
MPEYEIPKLQHYQQTLVLAQVVSIRDKVFQVLRKKINEGLLDSK

SEQ ID NO: 180 - protein, 33 aa, synthetic construct
  REGION 1..33: note = MLV-A_CT
MPEYEIPKLQHYQQTLVLAQVVSIRDKVFQVLR

SEQ ID NO: 181 - protein, 28 aa, synthetic construct
  REGION 1..28: note = MLV-A-RLess_CT
MLAQVVSIRDKVFQVLRKKINEGLLDSK

SEQ ID NO: 182 - protein, 17 aa, synthetic construct
  REGION 1..17: note = MLV-A-RLess_CT
MLAQVVSIRDKVFQVLR

SEQ ID NO: 183 - protein, 30 aa, synthetic construct
  REGION 1..30: note = VSV-G_CT
MKGLRNMEIDTYIQRKKTHKLKICLHIGVR

SEQ ID NO: 184 - protein, 41 aa, synthetic construct
  REGION 1..41: note = VSV-G_CT
MKGLRNMEIDTYIQRKKTHKLKICLHIGVRKKINEGLLDSK

SEQ ID NO: 185 - protein, 152 aa, synthetic construct
  REGION 1..152: note = HIV-1_gp41_MLLIR-LLP1-LLP3-LLP2-gp41-CT-N
MLLIRELGQRIRRPIHRIARYAAQLVEIVRDTGEAVAIATANLLNVASNKLEQSWYQLLNWWYKLAEWGRRGLLEVIRTVILLLDRLRHYSFLCLSRLDDWILALSGNVLRISRDRDREGGEEEIGEPRDPGRPIPLHTQFSLPSYGQRVRN

SEQ ID NO: 186 - protein, 163 aa, synthetic construct
  REGION 1..163: note = HIV-1_gp41_MLLIR-LLP1-LLP3-LLP2-gp41-CT-N
MLLIRELGQRIRRPIHRIARYAAQLVEIVRDTGEAVAIATANLLNVASNKLEQSWYQLLNWWYKLAEWGRRGLLEVIRTVILLLDRLRHYSFLCLSRLDDWILALSGNVLRISRDRDREGGEEEIGEPRDPGRPIPLHTQFSLPSYGQRVRNKKINEGLLDSK

SEQ ID NO: 187 - protein, 119 aa, synthetic construct
  REGION 1..119: note = HIV-1_gp41_LLP3-LLP2-gp41-CT-N
MAVAIATANLLNVASNKLEQSWYQLLNWWYKLAEWGRRGLLEVIRTVILLLDRLRHYSFLCLSRLDDWILALSGNVLRISRDRDREGGEEEIGEPRDPGRPIPLHTQFSLPSYGQRVRN

SEQ ID NO: 188 - protein, 81 aa, synthetic construct
  REGION 1..81: note = HIV-1_gp41_LLP2-gp41-CT-N
MLLEVIRTVILLLDRLRHYSFLCLSRLDDWILALSGNVLRISRDRDREGGEEEIGEPRDPGRPIPLHTQFSLPSYGQRVRN

SEQ ID NO: 189 - protein, 48 aa, synthetic construct
  REGION 1..48: note = HIV-1_gp41_gp41-CT-N
MSGNVLRISRDRDREGGEEEIGEPRDPGRPIPLHTQFSLPSYGQRVRN

SEQ ID NO: 190 - protein, 105 aa, synthetic construct
  REGION 1..105: note = HIV-1_gp41_MLLIR-LLP1-LLP3-LLP2
MLLIRELGQRIRRPIHRIARYAAQLVEIVRDTGEAVAIATANLLNVASNKLEQSWYQLLNWWYKLAEWGRRGLLEVIRTVILLLDRLRHYSFLCLSRLDDWILAL

SEQ ID NO: 191 - protein, 101 aa, synthetic construct
  REGION 1..101: note = HIV-1_gp41_LLP1-LLP3-LLP2
MELGQRIRRPIHRIARYAAQLVEIVRDTGEAVAIATANLLNVASNKLEQSWYQLLNWWYKLAEWGRRGLLEVIRTVILLLDRLRHYSFLCLSRLDDWILAL

SEQ ID NO: 192 - protein, 72 aa, synthetic construct
  REGION 1..72: note = HIV-1_gp41_LLP3-LLP2
MAVAIATANLLNVASNKLEQSWYQLLNWWYKLAEWGRRGLLEVIRTVILLLDRLRHYSFLCLSRLDDWILAL

SEQ ID NO: 193 - protein, 34 aa, synthetic construct
  REGION 1..34: note = HIV-1_gp41_LLP2
MLLEVIRTVILLLDRLRHYSFLCLSRLDDWILAL

SEQ ID NO: 194 - protein, 68 aa, synthetic construct
  REGION 1..68: note = HIV-1_gp41_MLLIR-LLP1-LLP3
MLLIRELGQRIRRPIHRIARYAAQLVEIVRDTGEAVAIATANLLNVASNKLEQSWYQLLNWWYKLAEW

SEQ ID NO: 195 - protein, 64 aa, synthetic construct
  REGION 1..64: note = HIV-1_gp41_LLP1-LLP3
MELGQRIRRPIHRIARYAAQLVEIVRDTGEAVAIATANLLNVASNKLEQSWYQLLNWWYKLAEW

SEQ ID NO: 196 - protein, 35 aa, synthetic construct
  REGION 1..35: note = HIV-1_gp41_LLP3
MAVAIATANLLNVASNKLEQSWYQLLNWWYKLAEW

SEQ ID NO: 197 - protein, 30 aa, synthetic construct
  REGION 1..30: note = HIV-1_gp41_MLLIR-LLP1
MLLIRELGQRIRRPIHRIARYAAQLVEIVR

SEQ ID NO: 198 - protein, 26 aa, synthetic construct
  REGION 1..26: note = HIV-1_gp41_LLP1
MELGQRIRRPIHRIARYAAQLVEIVR

SEQ ID NO: 199 - protein, 5 aa, synthetic construct
  REGION 1..5: note = HIV-1_gp41_MLLIR
MLLIR

SEQ ID NO: 200 - protein, 11 aa, synthetic construct
  REGION 1..11: note = CD63_CT_1to11
MAVEGGMKCVK

SEQ ID NO: 201 - protein, 10 aa, synthetic construct
  REGION 1..10: note = CD63_CT_73to81
MCGACKENYC

SEQ ID NO: 202 - protein, 14 aa, synthetic construct
  REGION 1..14: note = CD63_CT_225to238
MVEYGSRISKVLCC

SEQ ID NO: 203 - protein, 22 aa, synthetic construct
  REGION 1..22: note = CD63_CT_1to11
MAVEGGMKCVKKKINEGLLDSK

SEQ ID NO: 204 - protein, 21 aa, synthetic construct
  REGION 1..21: note = CD63_CT_73to81
MCGACKENYCKKINEGLLDSK

SEQ ID NO: 205 - protein, 25 aa, synthetic construct
  REGION 1..25: note = CD63_CT_225to238
MVEYGSRISKVLCCKKINEGLLDSK

SEQ ID NO: 206 - protein, 48 aa, synthetic construct
  REGION 1..48: note = CD29_CT
MKGEYKPNVVTTVASKYIPNEGTDWKANMKEKEFKAFERRDHIIMLLK

SEQ ID NO: 207 - protein, 59 aa, synthetic construct
  REGION 1..59: note = CD29_CT
MKGEYKPNVVTTVASKYIPNEGTDWKANMKEKEFKAFERRDHIIMLLKKKINEGLLDSK

SEQ ID NO: 208 - protein, 569 aa, synthetic construct
  REGION 1..569: note = MvH_CT_12
MVINREHLMIDRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 209 - protein, 573 aa, synthetic construct
  REGION 1..573: note = MvH_CT_16
MGSRIVINREHLMIDRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 210 - protein, 577 aa, synthetic construct
  REGION 1..577: note = MvH_CT_20
MPHPKGSRIVINREHLMIDRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 211 - protein, 576 aa, synthetic construct
  REGION 1..576: note = NivG_CT_19
MYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 212 - protein, 565 aa, synthetic construct
  REGION 1..565: note = BatPV_G_CT_8
MKNQNDHYILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 213 - protein, 565 aa, synthetic construct
  REGION 1..565: note = SeV_CT_08
MDSERSGKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 214 - protein, 574 aa, synthetic construct
  REGION 1..574: note = MLV-A-RLess_CT
MLAQVVSIRDKVFQVLRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 215 - protein, 562 aa, synthetic construct
  REGION 1..562: note = HIV-1_gp41_MLLIR
MLLIRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 216 - protein, 568 aa, synthetic construct
  REGION 1..568: note = CD63_CT_1to11
MAVEGGMKCVKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 217 - protein, 567 aa, synthetic construct
  REGION 1..567: note = CD63_CT_73to81
MCGACKENYCILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 218 - protein, 569 aa, synthetic construct
  REGION 1..569: note = HevG_CT_12
MKKINDGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 219 - protein, 590 aa, synthetic construct
  REGION 1..590: note = MLV-A_CT
MPEYEIPKLQHYQQTLVLAQVVSIRDKVFQVLRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 220 - protein, 563 aa, synthetic construct
  REGION 1..563: note = NivG_CT_06
MLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 221 - protein, 570 aa, synthetic construct
  REGION 1..570: note = NivG_CT_13
MIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 222 - protein, 569 aa, synthetic construct
  REGION 1..569: note = BatPV_G_CT_12
MWKKQKNQNDHYILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 223 - protein, 563 aa, synthetic construct
  REGION 1..563: note = HPIV2-HN_CT_06
MRIIFRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 224 - protein, 569 aa, synthetic construct
  REGION 1..569: note = HPIV2-HN_CT_12
MIPKRTCRIIFRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 225 - protein, 569 aa, synthetic construct
  REGION 1..569: note = CD63_CT_1to11
MKLVSDSERSGKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 226 - protein, 524 aa, synthetic construct
  REGION 1..524: note = Truncated NiV fusion glycoprotein (FcDelta22) at cytoplasmic tail
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 227 - protein, 498 aa, synthetic construct
  REGION 1..498: note = Truncated mature NiV fusion glycoprotein (FcDelta22) at cytoplasmic tail
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 228 - protein, 569 aa, synthetic construct
  REGION 1..569: note = NiVG protein attachment glycoprotein
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 229 - protein, 33 aa, synthetic construct
  REGION 1..33: note = Moloney murine leukemia virus cytoplasmic tail (632-665 of UniProt P03385; R-peptide 650-665)
NRLVQFVKDRISVVQALVLTQQYHQLKPIEYEP

SEQ ID NO: 230 - protein, 5 aa, synthetic construct
  REGION 1..5: note = peptide linker
GGGGS

SEQ ID NO: 231 - protein, 6 aa, synthetic construct
  REGION 1..6: note = peptide linker
GGGGGS

SEQ ID NO: 232 - protein, 5 aa, synthetic construct
  REGION 1..5: note = peptide linker
  REPEAT 1..5: note = Repeat is 1 to 10
GGGGS

SEQ ID NO: 233 - protein, 6 aa, synthetic construct
  REGION 1..6: note = peptide linker
  REPEAT 1..6: note = Repeat is 1 to 6
GGGGGS

SEQ ID NO: 234 - protein, 546 aa, synthetic construct
  REGION 1..546: note = Hendra F
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 235 - protein, 546 aa, synthetic construct
  REGION 1..546: note = Nipah F0
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 236 - protein, 557 aa, synthetic construct
  REGION 1..557: note = CedV F
MSNKRTTVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 237 - protein, 545 aa, synthetic construct
  REGION 1..545: note = Mojiang F
MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 238 - protein, 662 aa, synthetic construct
  REGION 1..662: note = Bat PV F
MKKKTDNPTISKRGHNHSRGIKSRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 239 - protein, 245 aa, synthetic construct
  REGION 1..245: note = Anti-CD19 scFv (FMC63)
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 240 - protein, 242 aa, synthetic construct
  REGION 1..242: note = Anti-CD19 scFv (FMC63)
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 241 - protein, 12 aa, synthetic construct
  REGION 1..12: note = IgG4 Hinge
ESKYGPPCPPCP

SEQ ID NO: 242 - protein, 25 aa, synthetic construct
  REGION 1..25: note = CD8 Hinge
TTTPAPRPPTPAPTIASQPLSLRPE

SEQ ID NO: 243 - protein, 39 aa, synthetic construct
  REGION 1..39: note = CD28
IEVMYPPPYLDNEKSNGTIIHVKGKHLCPSPLFPGPSKP

SEQ ID NO: 244 - protein, 44 aa, synthetic construct
  REGION 1..44: note = CD8
ACRPAAGGAVHTRGLDFACDIYIWAPLAGTCGVLLLSLVITLYC

SEQ ID NO: 245 - protein, 27 aa, synthetic construct
  REGION 1..27: note = CD28
FWVLVVVGGVLACYSLLVTVAFIIFWV

SEQ ID NO: 246 - protein, 27 aa, synthetic construct
  REGION 1..27: note = CD28
FWVLVVVGGVLACYSLLVTVAFIIFWV

SEQ ID NO: 247 - protein, 41 aa, synthetic construct
  REGION 1..41: note = CD28
RSKRSRLLHSDYMNMTPRRPGPTRKHYQPYAPPRDFAAYRS

SEQ ID NO: 248 - protein, 42 aa, synthetic construct
  REGION 1..42: note = 4-1BB
KRGRKKLLYIFKQPFMRPVQTTQEEDGCSCRFPEEEEGGCEL

SEQ ID NO: 249 - protein, 112 aa, synthetic construct
  REGION 1..112: note = CD3zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 250 - protein, 112 aa, synthetic construct
  REGION 1..112: note = CD3zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 251 - protein, 120 aa, synthetic construct
  REGION 1..120: note = P04578 (512-856)
AVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEW

SEQ ID NO: 252 - protein, 225 aa, synthetic construct
  REGION 1..225: note = HIV -1 Env (GP41)
DREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 253 - protein, 345 aa, synthetic construct
  REGION 1..345: note = HIV-1 NL4-3 Env (GP41)
AVGIGALFLGFLGAAGSTMGAASMTLTVQARQLLSDIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSIVNRVRQGYSPLSFQTHLPIPRGPDRPEGIEEEGGERGRDRSIRLVNGSLALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLERILL

SEQ ID NO: 254 - protein, 11 aa, synthetic construct
  REGION 1..11: note = Cytoplasmic tail amino acids
KKINEGLLDSK

SEQ ID NO: 255 - protein, 520 aa, synthetic construct
  REGION 1..520: note = Hendra virus F Protein, Without signal sequence
ILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 256 - protein, 520 aa, synthetic construct
  REGION 1..520: note = Nipah virus F Protein, without signal sequence
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 257 - protein, 551 aa, synthetic construct
  REGION 1..551: note = Cedar Virus F Protein, without signal sequence
TVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 258 - protein, 519 aa, synthetic construct
  REGION 1..519: note = Mojiang virus, Tongguan 1 F Protein, without signal sequence
IHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 259 - protein, 640 aa, synthetic construct
  REGION 1..640: note = Bat Paramyxovirus F Protein, without signal sequence
SRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 260 - protein, 8 aa, synthetic construct
  REGION 1..8: note = CDR-H1
GYTFSNYW

SEQ ID NO: 261 - protein, 8 aa, synthetic construct
  REGION 1..8: note = CDR-H2
ILPGSGST

SEQ ID NO: 262 - protein, 13 aa, synthetic construct
  REGION 1..13: note = CDR-H3
ARRGYGYDEGFDY

SEQ ID NO: 263 - protein, 6 aa, synthetic construct
  REGION 1..6: note = CDR-L1
QDINSY

SEQ ID NO: 264 - skipped

SEQ ID NO: 265 - protein, 9 aa, synthetic construct
  REGION 1..9: note = CDR-L3
LQYDEFPPT

SEQ ID NO: 266 - protein, 5 aa, synthetic construct
  REGION 1..5: note = CDR-H1
NYWIE

SEQ ID NO: 267 - protein, 17 aa, synthetic construct
  REGION 1..17: note = CDR-H2
EILPGSGSTSYNEKFKG

SEQ ID NO: 268 - protein, 11 aa, synthetic construct
  REGION 1..11: note = CDR-H3
RGYGYDEGFDY

SEQ ID NO: 269 - protein, 11 aa, synthetic construct
  REGION 1..11: note = CDR-L1
KASQDINSYLS

SEQ ID NO: 270 - protein, 7 aa, synthetic construct
  REGION 1..7: note = CDR-L2
RANRLVD

SEQ ID NO: 271 - protein, 7 aa, synthetic construct
  REGION 1..7: note = CDR-H1
GYTFSNY

SEQ ID NO: 272 - protein, 6 aa, synthetic construct
  REGION 1..6: note = CDR-H2
LPGSGS

SEQ ID NO: 273 - protein, 120 aa, synthetic construct
  REGION 1..120: note = VH
QVQLQQSGAELMKPGASVKMSCKATGYTFSNYWIEWVKQRPGHGLEWIGEILPGSGSTSYNEKFKGKATFTADTSSSTAYMQLSSLTSEDSAVYYCARRGYGYDEGFDYWGQGSTLTVSS

SEQ ID NO: 274 - protein, 108 aa, synthetic construct
  REGION 1..108: note = VL
DIKMTQSPSSMYASLGERVTITCKASQDINSYLSWFQQKPGKSPKTLIYRANRLVDGVPSRFSGSGSGQDYSLTISSLEYEDMGIYYCLQYDEFPPTFGAGTKLELKR

SEQ ID NO: 275 - protein, 15 aa, synthetic construct
  REGION 1..15: note = linker
GGGGSGGGGSGGGGS

SEQ ID NO: 276 - protein, 243 aa, synthetic construct
  REGION 1..243: note = scFv
QVQLQQSGAELMKPGASVKMSCKATGYTFSNYWIEWVKQRPGHGLEWIGEILPGSGSTSYNEKFKGKATFTADTSSSTAYMQLSSLTSEDSAVYYCARRGYGYDEGFDYWGQGSTLTVSSGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDEFPPTFGAGTKLELKR

SEQ ID NO: 277 - protein, 8 aa, synthetic construct
  REGION 1..8: note = CDR-H1
GYTFTDYV

SEQ ID NO: 278 - protein, 8 aa, synthetic construct
  REGION 1..8: note = CDR-H2
IYPGSGSS

SEQ ID NO: 279 - protein, 10 aa, synthetic construct
  REGION 1..10: note = CDR-H3
ARPGDLGFAY

SEQ ID NO: 280 - protein, 10 aa, synthetic construct
  REGION 1..10: note = CDR-L1
QSVDYDGDSY

SEQ ID NO: 281 - skipped

SEQ ID NO: 282 - protein, 9 aa, synthetic construct
  REGION 1..9: note = CDR-L3
QQSNKDPFT

SEQ ID NO: 283 - protein, 5 aa, synthetic construct
  REGION 1..5: note = CDR-H1
DYVIS

SEQ ID NO: 284 - protein, 17 aa, synthetic construct
  REGION 1..17: note = CDR-H2
EIYPGSGSSYYNEKFKG

SEQ ID NO: 285 - protein, 8 aa, synthetic construct
  REGION 1..8: note = CDR-H3
PGDLGFAY

SEQ ID NO: 286 - protein, 15 aa, synthetic construct
  REGION 1..15: note = CDR-L1
KASQSVDYDGDSYMN

SEQ ID NO: 287 - protein, 7 aa, synthetic construct
  REGION 1..7: note = CDR-L2
AASNLES

SEQ ID NO: 288 - protein, 9 aa, synthetic construct
  REGION 1..9: note = CDR-L3
QQSNKDPFT

SEQ ID NO: 289 - protein, 7 aa, synthetic construct
  REGION 1..7: note = CDR-H1
GYTFTDY

SEQ ID NO: 290 - protein, 6 aa, synthetic construct
  REGION 1..6: note = CDR-H2
YPGSGS

SEQ ID NO: 291 - protein, 117 aa, synthetic construct
  REGION 1..117: note = VH
QVQLQQSGPELVKPGASVKMSCKASGYTFTDYVISWVRQAPGQGLEWIGEIYPGSGSSYYNEKFKGRATLTADKSSNTAYMQLSSLRSEDSAVYFCARPGDLGFAYWGQGTLVTVSS

SEQ ID NO: 292 - protein, 112 aa, synthetic construct
  REGION 1..112: note = VL
DIVLTQSPSSLAVSLGQRATISCKASQSVDYDGDSYMNWYQQKPGQPPKLLIYAASNLESGIPARFSGSGSGTDFTLTIHPVEEEDAATYYCQQSNKDPFTFGGGTKLELKR

SEQ ID NO: 293 - protein, 244 aa, synthetic construct
  REGION 1..244: note = scFv
QVQLQQSGPELVKPGASVKMSCKASGYTFTDYVISWVRQAPGQGLEWIGEIYPGSGSSYYNEKFKGRATLTADKSSNTAYMQLSSLRSEDSAVYFCARPGDLGFAYWGQGTLVTVSSGGGGSGGGGSGGGGSDIVLTQSPSSLAVSLGQRATISCKASQSVDYDGDSYMNWYQQKPGQPPKLLIYAASNLESGIPARFSGSGSGTDFTLTIHPVEEEDAATYYCQQSNKDPFTFGGGTKLELKR

SEQ ID NO: 294 - protein, 8 aa, synthetic construct
  REGION 1..8: note = CDR-H1
GGTFSSYA

SEQ ID NO: 295 - protein, 8 aa, synthetic construct
  REGION 1..8: note = CDR-H2
INPNSGGT

SEQ ID NO: 296 - protein, 11 aa, synthetic construct
  REGION 1..11: note = CDR-H3
ARDGYSGSYSD

SEQ ID NO: 297 - protein, 12 aa, synthetic construct
  REGION 1..12: note = CDR-L1
QSVLSSSYNKNY

SEQ ID NO: 298 - skipped

SEQ ID NO: 299 - protein, 9 aa, synthetic construct
  REGION 1..9: note = CDR-L3
QQYYSTPWT

SEQ ID NO: 300 - protein, 5 aa, synthetic construct
  REGION 1..5: note = CDR-H1
SYAIS

SEQ ID NO: 301 - protein, 17 aa, synthetic construct
  REGION 1..17: note = CDR-H2
WINPNSGGTNYAQKFQG

SEQ ID NO: 302 - protein, 9 aa, synthetic construct
  REGION 1..9: note = CDR-H3
DGYSGSYSD

SEQ ID NO: 303 - protein, 17 aa, synthetic construct
  REGION 1..17: note = CDR-L1
KSSQSVLSSSYNKNYLA

SEQ ID NO: 304 - protein, 7 aa, synthetic construct
  REGION 1..7: note = CDR-L2
WASTRES

SEQ ID NO: 305 - protein, 7 aa, synthetic construct
  REGION 1..7: note = CDR-H1
GGTFSSY

SEQ ID NO: 306 - protein, 6 aa, synthetic construct
  REGION 1..6: note = CDR-H2
NPNSGG

SEQ ID NO: 307 - protein, 118 aa, synthetic construct
  REGION 1..118: note = VH
QVQLVQSGAEVKKPGASVKVSCKASGGTFSSYAISWVRQAPGQGLEWMGWINPNSGGTNYAQKFQGRVTMTRDTSTSTVYMELSSLRSEDTAVYYCARDGYSGSYSDWGQGTLVTVSS

SEQ ID NO: 308 - protein, 113 aa, synthetic construct
  REGION 1..113: note = VL
DIVMTQSPDSLAVSLGERATINCKSSQSVLSSSYNKNYLAWYQQKPGQPPKLLIYWASTRESGVPDRFSGSGSGTDFTLTISSLQAEDVAVYYCQQYYSTPWTFGQGTKVEIK

SEQ ID NO: 309 - protein, 246 aa, synthetic construct
  REGION 1..246: note = scFv
QVQLVQSGAEVKKPGASVKVSCKASGGTFSSYAISWVRQAPGQGLEWMGWINPNSGGTNYAQKFQGRVTMTRDTSTSTVYMELSSLRSEDTAVYYCARDGYSGSYSDWGQGTLVTVSSGGGGSGGGGSGGGGSDIVMTQSPDSLAVSLGERATINCKSSQSVLSSSYNKNYLAWYQQKPGQPPKLLIYWASTRESGVPDRFSGSGSGTDFTLTISSLQAEDVAVYYCQQYYSTPWTFGQGTKVEIK

SEQ ID NO: 310 - protein, 8 aa, synthetic construct
  REGION 1..8: note = CDR-H1
GYTFTSYD

SEQ ID NO: 311 - protein, 8 aa, synthetic construct
  REGION 1..8: note = CDR-H2
IIPLSGAP

SEQ ID NO: 312 - protein, 15 aa, synthetic construct
  REGION 1..15: note = CDR-H3
ARGALYNWNDGWFDP

SEQ ID NO: 313 - protein, 6 aa, synthetic construct
  REGION 1..6: note = CDR-L1
QDIGDY

SEQ ID NO: 314 - skipped

SEQ ID NO: 315 - protein, 9 aa, synthetic construct
  REGION 1..9: note = CDR-L3
QQANSFPLT

SEQ ID NO: 316 - protein, 5 aa, synthetic construct
  REGION 1..5: note = CDR-H1
SYDIN

SEQ ID NO: 317 - protein, 17 aa, synthetic construct
  REGION 1..17: note = CDR-H2
GIIPLSGAPNYAHKFQG

SEQ ID NO: 318 - protein, 13 aa, synthetic construct
  REGION 1..13: note = CDR-H3
GALYNWNDGWFDP

SEQ ID NO: 319 - protein, 11 aa, synthetic construct
  REGION 1..11: note = CDR-L1
RASQDIGDYLA

SEQ ID NO: 320 - protein, 7 aa, synthetic construct
  REGION 1..7: note = CDR-L2
DASSLQS

SEQ ID NO: 321 - protein, 7 aa, synthetic construct
  REGION 1..7: note = CDR-H1
GYTFTSY

SEQ ID NO: 322 - protein, 6 aa, synthetic construct
  REGION 1..6: note = CDR-H2
IPLSGA

SEQ ID NO: 323 - protein, 122 aa, synthetic construct
  REGION 1..122: note = VH
QVQLVQSGAEVKKPGSSVKVSCKASGYTFTSYDINWVRQAPGQGLEWMGGIIPLSGAPNYAHKFQGRVTITADESTSTAYMELSSLRSEDTAVYYCARGALYNWNDGWFDPWGQGTLVTVSS

SEQ ID NO: 324 - protein, 107 aa, synthetic construct
  REGION 1..107: note = VL
DIQMTQSPSSLSASVGDRVTITCRASQDIGDYLAWYQQKPGKAPKLLIYDASSLQSGVPSRFSGSGSGTDFTLTISSLQPEDFATYYCQQANSFPLTFGGGTKVEIK

SEQ ID NO: 325 - protein, 244 aa, synthetic construct
  REGION 1..244: note = scFv
QVQLVQSGAEVKKPGSSVKVSCKASGYTFTSYDINWVRQAPGQGLEWMGGIIPLSGAPNYAHKFQGRVTITADESTSTAYMELSSLRSEDTAVYYCARGALYNWNDGWFDPWGQGTLVTVSSGGGGSGGGGSGGGGSDIQMTQSPSSLSASVGDRVTITCRASQDIGDYLAWYQQKPGKAPKLLIYDASSLQSGVPSRFSGSGSGTDFTLTISSLQPEDFATYYCQQANSFPLTFGGGTKVEIK

SEQ ID NO: 326 - protein, 8 aa, synthetic construct
  REGION 1..8: note = CDR-H1
GDTFSDHA

SEQ ID NO: 327 - protein, 8 aa, synthetic construct
  REGION 1..8: note = CDR-H2
MNPKIGNT

SEQ ID NO: 328 - protein, 12 aa, synthetic construct
  REGION 1..12: note = CDR-H3
VYDSSGYDAFDI

SEQ ID NO: 329 - protein, 12 aa, synthetic construct
  REGION 1..12: note = CDR-L1
QSVLSTSYNRNF

SEQ ID NO: 330 - skipped

SEQ ID NO: 331 - protein, 9 aa, synthetic construct
  REGION 1..9: note = CDR-L3
QQYYSTPYT

SEQ ID NO: 332 - protein, 5 aa, synthetic construct
  REGION 1..5: note = CDR-H1
DHAIN

SEQ ID NO: 333 - protein, 17 aa, synthetic construct
  REGION 1..17: note = CDR-H2
WMNPKIGNTGYAQKFQG

SEQ ID NO: 334 - protein, 10 aa, synthetic construct
  REGION 1..10: note = CDR-H3
DSSGYDAFDI

SEQ ID NO: 335 - protein, 17 aa, synthetic construct
  REGION 1..17: note = CDR-L1
KSSQSVLSTSYNRNFLA

SEQ ID NO: 336 - protein, 7 aa, synthetic construct
  REGION 1..7: note = CDR-L2
WASTRQS

SEQ ID NO: 337 - protein, 7 aa, synthetic construct
  REGION 1..7: note = CDR-H1
GDTFSDH

SEQ ID NO: 338 - protein, 6 aa, synthetic construct
  REGION 1..6: note = CDR-H2
NPKIGN

SEQ ID NO: 339 - protein, 119 aa, synthetic construct
  REGION 1..119: note = VH
QVQLVQSGAEVKKPGASVKVSCKASGDTFSDHAINWVRQAPGQGLEWMGWMNPKIGNTGYAQKFQGRVTMTRDTSTSTVYMELSSLRSEDTAVYYCVYDSSGYDAFDIWGQGTTVTVSS

SEQ ID NO: 340 - protein, 113 aa, synthetic construct
  REGION 1..113: note = VL
DIVMTQSPDSLAVSLGERATINCKSSQSVLSTSYNRNFLAWYQQKPGQPPKLLIYWASTRQSGVPDRFSGSGSGTDFTLTISSLQAEDVAVYYCQQYYSTPYTFGQGTKLEIK

SEQ ID NO: 341 - protein, 247 aa, synthetic construct
  REGION 1..247: note = scFv
QVQLVQSGAEVKKPGASVKVSCKASGDTFSDHAINWVRQAPGQGLEWMGWMNPKIGNTGYAQKFQGRVTMTRDTSTSTVYMELSSLRSEDTAVYYCVYDSSGYDAFDIWGQGTTVTVSSGGGGSGGGGSGGGGSDIVMTQSPDSLAVSLGERATINCKSSQSVLSTSYNRNFLAWYQQKPGQPPKLLIYWASTRQSGVPDRFSGSGSGTDFTLTISSLQAEDVAVYYCQQYYSTPYTFGQGTKLEIK

SEQ ID NO: 342 - protein, 8 aa, synthetic construct
  REGION 1..8: note = CDR-H1
GYSLITHW

SEQ ID NO: 343 - protein, 8 aa, synthetic construct
  REGION 1..8: note = CDR-H2
INPSDGVT

SEQ ID NO: 344 - protein, 11 aa, synthetic construct
  REGION 1..11: note = CDR-H3
AREYYGEGFDY

SEQ ID NO: 345 - protein, 6 aa, synthetic construct
  REGION 1..6: note = CDR-L1
QGISNY

SEQ ID NO: 346 - skipped

SEQ ID NO: 347 - protein, 9 aa, synthetic construct
  REGION 1..9: note = CDR-L3
QQSYSTPLT

SEQ ID NO: 348 - protein, 5 aa, synthetic construct
  REGION 1..5: note = CDR-H1
THWMH

SEQ ID NO: 349 - protein, 17 aa, synthetic construct
  REGION 1..17: note = CDR-H2
MINPSDGVTYYAQTFQG

SEQ ID NO: 350 - protein, 9 aa, synthetic construct
  REGION 1..9: note = CDR-H3
EYYGEGFDY

SEQ ID NO: 351 - protein, 11 aa, synthetic construct
  REGION 1..11: note = CDR-L1
RASQGISNYLA

SEQ ID NO: 352 - protein, 7 aa, synthetic construct
  REGION 1..7: note = CDR-L2
SASNLQS

SEQ ID NO: 353 - protein, 7 aa, synthetic construct
  REGION 1..7: note = CDR-H1
GYSLITH

SEQ ID NO: 354 - protein, 6 aa, synthetic construct
  REGION 1..6: note = CDR-H2
NPSDGV

SEQ ID NO: 355 - protein, 118 aa, synthetic construct
  REGION 1..118: note = VH
QVQLVQSGAEVKKPGASVKVSCKASGYSLITHWMHWVRQAPGQGLEWMGMINPSDGVTYYAQTFQGRVTMTRDTSTSTVYMELSSLRSEDTAVYYCAREYYGEGFDYWGQGTLVTVSS

SEQ ID NO: 356 - protein, 108 aa, synthetic construct
  REGION 1..108: note = VL
DIQMTQSPSSLSASVGDRVTITCRASQGISNYLAWYQQKPGKAPKLLIYSASNLQSGVPSRFSGSGSGTDFTLTISSLQPEDFATYYCQQSYSTPLTFGGGTKVEIKR

SEQ ID NO: 357 - protein, 241 aa, synthetic construct
  REGION 1..241: note = scFv
QVQLVQSGAEVKKPGASVKVSCKASGYSLITHWMHWVRQAPGQGLEWMGMINPSDGVTYYAQTFQGRVTMTRDTSTSTVYMELSSLRSEDTAVYYCAREYYGEGFDYWGQGTLVTVSSGGGGSGGGGSGGGGSDIQMTQSPSSLSASVGDRVTITCRASQGISNYLAWYQQKPGKAPKLLIYSASNLQSGVPSRFSGSGSGTDFTLTISSLQPEDFATYYCQQSYSTPLTFGGGTKVEIKR

SEQ ID NO: 358 - protein, 8 aa, synthetic construct
  REGION 1..8: note = CDR-H1
GFTFSGYW

SEQ ID NO: 359 - protein, 8 aa, synthetic construct
  REGION 1..8: note = CDR-H2
ISPGGGST

SEQ ID NO: 360 - protein, 14 aa, synthetic construct
  REGION 1..14: note = CDR-H3
ASSLTATHTYEYDY

SEQ ID NO: 361 - protein, 5 aa, synthetic construct
  REGION 1..5: note = CDR-H1
GYWMY

SEQ ID NO: 362 - protein, 17 aa, synthetic construct
  REGION 1..17: note = CDR-H2
AISPGGGSTYYPDSVKG

SEQ ID NO: 363 - protein, 12 aa, synthetic construct
  REGION 1..12: note = CDR-H3
SLTATHTYEYDY

SEQ ID NO: 364 - protein, 7 aa, synthetic construct
  REGION 1..7: note = CDR-H1
GFTFSGY

SEQ ID NO: 365 - protein, 6 aa, synthetic construct
  REGION 1..6: note = CDR-H2
SPGGGS

SEQ ID NO: 366 - protein, 121 aa, synthetic construct
  REGION 1..121: note = VHH
EVQLVESGGGLVQSGGSLRLSCAASGFTFSGYWMYWVRQAPGKGLEWVSAISPGGGSTYYPDSVKGRFTISRDNAKNTLYLQMNSLEPEDTALYYCASSLTATHTYEYDYWGQGTQVTVSS

SEQ ID NO: 367 - protein, 45 aa, synthetic construct
  REGION 1..45: note = CD8a hinge domain
TTTPAPRPPTPAPTIASQPLSLRPEACRPAAGGAVHTRGLDFACD

SEQ ID NO: 368 - protein, 24 aa, synthetic construct
  REGION 1..24: note = CD8a transmembrane domain
IYIWAPLAGTCGVLLLSLVITLYC

SEQ ID NO: 369 - protein, 122 aa, synthetic construct
  REGION 1..122: note = CD8_1 VH
QVQLVQSGAEVKKPGASVKVSCKASGGTFSSYAISWVRQAPGQGLEWMGIIDPSDGNTNYAQNFQGRVTMTRDTSTSTVYMELSSLRSEDTAVYYCAKERAAAGYYYYMDVWGQGTTVTVSS

SEQ ID NO: 370 - protein, 108 aa, synthetic construct
  REGION 1..108: note = CD8_1 VL
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 371 - protein, 119 aa, synthetic construct
  REGION 1..119: note = CD8_2 VH
QVQLVQSGAEVKKPGASVKVSCKASGYTFTDYYIQWVRQAPGQGLEWMGWINPNSGGTSYAQKFQGRVTMTRDTSTSTVYMELSSLRSEDTAVYYCAKEGDYYYGMDAWGQGTMVTVSS

SEQ ID NO: 372 - protein, 113 aa, synthetic construct
  REGION 1..113: note = CD8_2 VL
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTPHTFGQGTKVEIKR

SEQ ID NO: 373 - protein, 117 aa, synthetic construct
  REGION 1..117: note = CD8_3 VH
QVQLVQSGAEVKKPGASVKVSCKASGYTFTSYYMHWVRQAPGQGLEWMGGFDPEDGETIYAQKFQGRVTMTRDTSTSTVYMELSSLRSEDTAVYYCARDQGWGMDVWGQGTTVTVSS

SEQ ID NO: 374 - protein, 108 aa, synthetic construct
  REGION 1..108: note = CD8_3 VL
DIQMTQSPSSLSASVGDRVTITCRASQSISSYLNWYQQKPGKAPKLLIYAASSLQSGVPSRFSGSGSGTDFTLTISSLQPEDFATYYCQQTYSTPYTFGQGTKLEIKR

SEQ ID NO: 375 - protein, 118 aa, synthetic construct
  REGION 1..118: note = CD8_4 VH
QVQLVQSGAEVKKPGASVKVSCKASGYTFTNHYMHWVRQAPGQGLEWMGWMNPNSGNTGYAQKFQGRVTMTRDTSTSTVYMELSSLRSEDTAVYYCASSESGSDLDYWGQGTLVTVSS

SEQ ID NO: 376 - protein, 108 aa, synthetic construct
  REGION 1..108: note = CD8_4 VL
DIQMTQSPSSLSASVGDRVTITCRASQTIGNYVNWYQQKPGKAPKLLIYGASNLHTGVPSRFSGSGSGTDFTLTISSLQPEDFATYYCQQTYSAPLTFGGGTKVEIKR

SEQ ID NO: 377 - protein, 125 aa, synthetic construct
  REGION 1..125: note = VHH
QVQLVESGGGLVQAGGSLRLSCAASGRTFSGYVMGWFRQAPGKQRKFVAAISRGGLSTSYADSVKGRFTISRDNAKNTVFLQMNTLKPEDTAVYYCAADRSDLYEITAASNIDSWGQGTLVTVSS

SEQ ID NO: 378 - protein, 21 aa, synthetic construct
  REGION 1..21: note = CD8a signal peptide
MALPVTALLLPLALLLHAARP

SEQ ID NO: 379 - protein, 20 aa, synthetic construct
  REGION 1..20: note = IgK signal peptide
METDTLLLWVLLLWVPGSTG

SEQ ID NO: 380 - protein, 22 aa, synthetic construct
  REGION 1..22: note = GMCSFR-a (CSF2RA) signal peptide
MLLLVTSLLLCELPHPAFLLIP

SEQ ID NO: 381 - protein, 45 aa, synthetic construct; identical to SEQ ID NO: 367
  REGION 1..45: note = CD8a hinge domain

SEQ ID NO: 382 - protein, 39 aa, synthetic construct; identical to SEQ ID NO: 243
  REGION 1..39: note = CD28 hinge domain

SEQ ID NO: 383 - protein, 42 aa, synthetic construct
  REGION 1..42: note = CD28 hinge domain
AAAIEVMYPPPYLDNEKSNGTIIHVKGKHLCPSPLFPGPSKP

SEQ ID NO: 384 - protein, 12 aa, synthetic construct
  REGION 1..12: note = IgG4 hinge domain
ESKYGPPCPPCP

SEQ ID NO: 385 - protein, 12 aa, synthetic construct
  REGION 1..12: note = IgG4 hinge domain
ESKYGPPCPSCP

SEQ ID NO: 386 - protein, 229 aa, synthetic construct
  REGION 1..229: note = IgG4 hinge-CH2-CH3 domain
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 387 - protein, 24 aa, synthetic construct
  REGION 1..24: note = CD8a transmembrane domain
IYIWAPLAGTCGVLLLSLVITLYC

SEQ ID NO: 388 - protein, 27 aa, synthetic construct
  REGION 1..27: note = CD28 transmembrane domain
FWVLVVVGGVLACYSLLVTVAFIIFWV

SEQ ID NO: 389 - protein, 28 aa, synthetic construct
  REGION 1..28: note = CD28 transmembrane domain
MFWVLVVVGGVLACYSLLVTVAFIIFWV

SEQ ID NO: 390 - protein, 42 aa, synthetic construct; identical to SEQ ID NO: 248
  REGION 1..42: note = 4-1BB costimulatory domain

SEQ ID NO: 391 - protein, 41 aa, synthetic construct; identical to SEQ ID NO: 247
  REGION 1..41: note = CD28 costimulatory domain

SEQ ID NO: 392 - protein, 112 aa, synthetic construct; identical to SEQ ID NO: 249
  REGION 1..112: note = CD3z signaling domain

SEQ ID NO: 393 - protein, 112 aa, synthetic construct; identical to SEQ ID NO: 250
  REGION 1..112: note = CD3z signaling domain (with Q to K mutation at position 14)

SEQ ID NO: 394 - protein, 245 aa, synthetic construct; identical to SEQ ID NO: 239
  REGION 1..245: note = Anti-CD19 FMC63 scFv entire sequence, with Whitlow linker

SEQ ID NO: 395 - protein, 107 aa, synthetic construct
  REGION 1..107: note = Anti-CD19 FMC63 scFv light chain variable region
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 396 - protein, 6 aa, synthetic construct
  REGION 1..6: note = Anti-CD19 FMC63 scFv light chain CDR1
QDISKY

SEQ ID NO: 397 - skipped

SEQ ID NO: 398 - protein, 9 aa, synthetic construct
  REGION 1..9: note = Anti-CD19 FMC63 scFv light chain CDR3
QQGNTLPYT

SEQ ID NO: 399 - protein, 18 aa, synthetic construct
  REGION 1..18: note = Whitlow linker
GSTSGSGKPGSGEGSTKG

SEQ ID NO: 400 - protein, 120 aa, synthetic construct
  REGION 1..120: note = Anti-CD19 FMC63 scFv heavy chain variable region
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 401 - protein, 8 aa, synthetic construct
  REGION 1..8: note = Anti-CD19 FMC63 scFv heavy chain CDR1
GVSLPDYG

SEQ ID NO: 402 - protein, 7 aa, synthetic construct
  REGION 1..7: note = Anti-CD19 FMC63 scFv heavy chain CDR2
IWGSETT

SEQ ID NO: 403 - protein, 14 aa, synthetic construct
  REGION 1..14: note = Anti-CD19 FMC63 scFv heavy chain CDR3
AKHYYYGGSYAMDY

SEQ ID NO: 404 - protein, 242 aa, synthetic construct; identical to SEQ ID NO: 240
  REGION 1..242: note = Anti-CD19 FMC63 scFv entire sequence, with 3xG4S linker

SEQ ID NO: 405 - protein, 15 aa, synthetic construct
  REGION 1..15: note = 3xG4S linker
GGGGSGGGGSGGGGS

SEQ ID NO: 406 - DNA, 1467 bp, synthetic construct
  source 1..1467: mol_type = other DNA
  misc_feature 1..1467: note = Exemplary CD19 CAR nucleotide sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 407 - protein, 489 aa, synthetic construct
  REGION 1..489: note = Exemplary CD19 CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 408 - DNA, 1458 bp, synthetic construct
  source 1..1458: mol_type = other DNA
  misc_feature 1..1458: note = Tisagenlecleucel CD19 CAR nucleotide sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 409 - protein, 486 aa, synthetic construct
  REGION 1..486: note = Tisagenlecleucel CD19 CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 410 - DNA, 1383 bp, synthetic construct
  source 1..1383: mol_type = other DNA
  misc_feature 1..1383: note = Lisocabtagene maraleucel CD19 CAR nucleotide sequence
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 411 - protein, 461 aa, synthetic construct
  REGION 1..461: note = Lisocabtagene maraleucel CD19 CAR amino acid sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 412 - DNA, 1467 bp, synthetic construct
  source 1..1467: mol_type = other DNA
  misc_feature 1..1467: note = Axicabtagene ciloleucel CD19 CAR nucleotide sequence
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 413 - protein, 489 aa, synthetic construct
  REGION 1..489: note = Axicabtagene ciloleucel CD19 CAR amino acid sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 414 - protein, 246 aa, synthetic construct
  REGION 1..246: note = Anti-CD20 Leu16 scFv entire sequence, with Whitlow linker
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 415 - protein, 106 aa, synthetic construct
  REGION 1..106: note = Anti-CD20 Leu16 scFv light chain variable region
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 416 - protein, 10 aa, synthetic construct
  REGION 1..10: note = Anti-CD20 Leu16 scFv light chain CDR1
RASSSVNYMD

SEQ ID NO: 417 - protein, 7 aa, synthetic construct
  REGION 1..7: note = Anti-CD20 Leu16 scFv light chain CDR2
ATSNLAS

SEQ ID NO: 418 - protein, 9 aa, synthetic construct
  REGION 1..9: note = Anti-CD20 Leu16 scFv light chain CDR3
QQWSFNPPT

SEQ ID NO: 419 - protein, 122 aa, synthetic construct
  REGION 1..122: note = Anti-CD20 Leu16 scFv heavy chain
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 420 - protein, 5 aa, synthetic construct
  REGION 1..5: note = Anti-CD20 Leu16 scFv heavy chain CDR1
SYNMH

SEQ ID NO: 421 - protein, 17 aa, synthetic construct
  REGION 1..17: note = Anti-CD20 Leu16 scFv heavy chain CDR2
AIYPGNGDTSYNQKFKG

SEQ ID NO: 422 - protein, 13 aa, synthetic construct
  REGION 1..13: note = Anti-CD20 Leu16 scFv heavy chain CDR3
SNYYGSSYWFFDV

SEQ ID NO: 423 - protein, 246 aa, synthetic construct
  REGION 1..246: note = Anti-CD22 m971 scFv entire sequence, with 3xG4S linker
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 424 - protein, 124 aa, synthetic construct
  REGION 1..124: note = Anti-CD22 m971 scFv heavy chain variable region
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 425 - protein, 10 aa, synthetic construct
  REGION 1..10: note = Anti-CD22 m971 scFv heavy chain CDR1
GDSVSSNSAA

SEQ ID NO: 426 - protein, 9 aa, synthetic construct
  REGION 1..9: note = Anti-CD22 m971 scFv heavy chain CDR2
TYYRSKWYN

SEQ ID NO: 427 - protein, 14 aa, synthetic construct
  REGION 1..14: note = Anti-CD22 m971 scFv heavy chain CDR3
AREVTGDLEDAFDI

SEQ ID NO: 428 - protein, 107 aa, synthetic construct
  REGION 1..107: note = Anti-CD22 m971 scFv light chain
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 429 - protein, 6 aa, synthetic construct
  REGION 1..6: note = Anti-CD22 m971 scFv light chain CDR1
QTIWSY

SEQ ID NO: 430 - skipped

SEQ ID NO: 431 - protein, 9 aa, synthetic construct
  REGION 1..9: note = Anti-CD22 m971 scFv light chain CDR3
QQSYSIPQT

SEQ ID NO: 432 - protein, 246 aa, synthetic construct
  REGION 1..246: note = Anti-CD22 m971-L7 scFv entire sequence, with 3xG4S linker
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 433 - protein, 124 aa, synthetic construct
  REGION 1..124: note = Anti-CD22 m971-L7 scFv heavy chain variable region
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSS

SEQ ID NO: 434 - protein, 10 aa, synthetic construct
  REGION 1..10: note = Anti-CD22 m971-L7 scFv heavy chain CDR1
GDSVSSNSVA

SEQ ID NO: 435 - protein, 9 aa, synthetic construct
  REGION 1..9: note = Anti-CD22 m971-L7 scFv heavy chain CDR2
TYYRSTWYN

SEQ ID NO: 436 - protein, 14 aa, synthetic construct
  REGION 1..14: note = Anti-CD22 m971-L7 scFv heavy chain CDR3
AREVTGDLEDAFDI

SEQ ID NO: 437 - protein, 107 aa, synthetic construct
  REGION 1..107: note = Anti-CD22 m971-L7 scFv light chain variable region
DIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 438 - protein, 6 aa, synthetic construct
  REGION 1..6: note = Anti-CD22 m971-L7 scFv light chain CDR1
QTIWSY

SEQ ID NO: 439 - skipped

SEQ ID NO: 440 - protein, 9 aa, synthetic construct
  REGION 1..9: note = Anti-CD22 m971-L7 scFv light chain CDR3
QQSYSIPQT

SEQ ID NO: 441 - protein, 246 aa, synthetic construct
  REGION 1..246: note = Anti-BCMA C11D5.3 scFv entire sequence, with Whitlow linker
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 442 - protein, 111 aa, synthetic construct
  REGION 1..111: note = Anti-BCMA C11D5.3 scFv light chain variable region
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 443 - protein, 15 aa, synthetic construct
  REGION 1..15: note = Anti-BCMA C11D5.3 scFv light chain CDR1
RASESVSVIGAHLIH

SEQ ID NO: 444 - protein, 7 aa, synthetic construct
  REGION 1..7: note = Anti-BCMA C11D5.3 scFv light chain CDR2
LASNLET

SEQ ID NO: 445 - protein, 9 aa, synthetic construct
  REGION 1..9: note = Anti-BCMA C11D5.3 scFv light chain CDR3
LQSRIFPRT

SEQ ID NO: 446 - protein, 117 aa, synthetic construct
  REGION 1..117: note = Anti-BCMA C11D5.3 scFv heavy chain variable region
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 447 - protein, 5 aa, synthetic construct
  REGION 1..5: note = Anti-BCMA C11D5.3 scFv heavy chain CDR1
DYSIN

SEQ ID NO: 448 - protein, 17 aa, synthetic construct
  REGION 1..17: note = Anti-BCMA C11D5.3 scFv heavy chain CDR2
WINTETREPAYAYDFRG

SEQ ID NO: 449 - protein, 8 aa, synthetic construct
  REGION 1..8: note = Anti-BCMA C11D5.3 scFv heavy chain CDR3
DYSYAMDY

SEQ ID NO: 450 - protein, 246 aa, synthetic construct
  REGION 1..246: note = Anti-BCMA C12A3.2 scFv entire sequence, with Whitlow linker
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 451 - protein, 111 aa, synthetic construct
  REGION 1..111: note = Anti-BCMA C12A3.2 scFv light chain variable region
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 452 - protein, 15 aa, synthetic construct
  REGION 1..15: note = Anti-BCMA C12A3.2 scFv light chain CDR1
RASESVTILGSHLIY

SEQ ID NO: 453 - protein, 7 aa, synthetic construct
  REGION 1..7: note = Anti-BCMA C12A3.2 scFv light chain CDR2
LASNVQT

SEQ ID NO: 454 - protein, 9 aa, synthetic construct
  REGION 1..9: note = Anti-BCMA C12A3.2 scFv light chain CDR3
LQSRTIPRT

SEQ ID NO: 455 - protein, 117 aa, synthetic construct
  REGION 1..117: note = Anti-BCMA C12A3.2 scFv heavy chain variable region
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 456 - protein, 5 aa, synthetic construct
  REGION 1..5: note = Anti-BCMA C12A3.2 scFv heavy chain CDR1
HYSMN

SEQ ID NO: 457 - protein, 17 aa, synthetic construct
  REGION 1..17: note = Anti-BCMA C12A3.2 scFv heavy chain CDR2
RINTESGVPIYADDFKG

SEQ ID NO: 458 - protein, 8 aa, synthetic construct
  REGION 1..8: note = Anti-BCMA C12A3.2 scFv heavy chain CDR3
DYLYSLDF

SEQ ID NO: 459 - protein, 121 aa, synthetic construct
  REGION 1..121: note = Anti-BCMA FHVH33 entire sequence
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 460 - protein, 8 aa, synthetic construct
  REGION 1..8: note = Anti-BCMA FHVH33 CDR1
GFTFSSYA

SEQ ID NO: 461 - protein, 8 aa, synthetic construct
  REGION 1..8: note = Anti-BCMA FHVH33 CDR2
ISGSGDYI

SEQ ID NO: 462 - protein, 14 aa, synthetic construct
  REGION 1..14: note = Anti-BCMA FHVH33 CDR3
AKEGTGANSSLADY

SEQ ID NO: 463 - protein, 243 aa, synthetic construct
  REGION 1..243: note = Anti-BCMA CT103A scFv entire sequence, with Whitlow linker
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 464 - protein, 106 aa, synthetic construct
  REGION 1..106: note = Anti-BCMA CT103A scFv light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 465 - protein, 6 aa, synthetic construct
  REGION 1..6: note = Anti-BCMA CT103A scFv light chain CDR1
QSISSY

SEQ ID NO: 466 - skipped

SEQ ID NO: 467 - protein, 8 aa, synthetic construct
  REGION 1..8: note = Anti-BCMA CT103A scFv light chain CDR3
QQKYDLLT

SEQ ID NO: 468 - protein, 119 aa, synthetic construct
  REGION 1..119: note = Anti-BCMA CT103A scFv heavy chain variable region
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 469 - protein, 10 aa, synthetic construct
  REGION 1..10: note = Anti-BCMA CT103A scFv heavy chain CDR1
GGSISSSSYY

SEQ ID NO: 470 - protein, 7 aa, synthetic construct
  REGION 1..7: note = Anti-BCMA CT103A scFv heavy chain CDR2
ISYSGST

SEQ ID NO: 471 - protein, 11 aa, synthetic construct
  REGION 1..11: note = Anti-BCMA CT103A scFv heavy chain CDR3
ARDRGDTILDV

SEQ ID NO: 472 - DNA, 1503 bp, synthetic construct
  source 1..1503: mol_type = other DNA
  misc_feature 1..1503: note = Exemplary BCMA CAR nucleotide sequence
atggccttaccagtgaccgccttgctcctgccgctggccttgctgctccacgccgccaggccggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaaaatacgacctcctcacttttggcggagggaccaaggttgagatcaaaggcagcaccagcggctccggcaagcctggctctggcgagggcagcacaaagggacagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctcctatagtgggagcacctactacaacccgtccctcaagagtcgagtcaccatatccgtagacacgtccaagaaccagttctccctgaagctgagttctgtgaccgccgcagacacggcggtgtactactgcgccagagatcgtggagacaccatactagacgtatggggtcagggtacaatggtcaccgtcagctcattcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 473 - protein, 501 aa, synthetic construct
  REGION 1..501: note = Exemplary BCMA CAR amino acid sequence
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 474 - protein, 5 aa, synthetic construct
  REGION 1..5: note = CD3 CDR-H1
RYTMH

SEQ ID NO: 475 - protein, 17 aa, synthetic construct
  REGION 1..17: note = CD3 CDR-H2
YINPSRGYTNYNQKFKD

SEQ ID NO: 476 - protein, 10 aa, synthetic construct
  REGION 1..10: note = CD3 CDR-H3
YYDDHYCLDY

SEQ ID NO: 477 - protein, 10 aa, synthetic construct
  REGION 1..10: note = CD3 CDR-L1
RASSSVSYMN

SEQ ID NO: 478 - protein, 7 aa, synthetic construct
  REGION 1..7: note = CD3 CDR-L2
DTSKVAS

SEQ ID NO: 479 - protein, 9 aa, synthetic construct
  REGION 1..9: note = CD3 CDR-L3
QQWSSNPLT

SEQ ID NO: 480 - protein, 121 aa, synthetic construct
  REGION 1..121: note = CD3 VH
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVE

SEQ ID NO: 481 - protein, 106 aa, synthetic construct
  REGION 1..106: note = CD3 VL
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 482 - protein, 243 aa, synthetic construct
  REGION 1..243: note = CD3 scFv
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 483 - protein, 5 aa, synthetic construct
  REGION 1..5: note = CDR-H1
SYAIS

SEQ ID NO: 484 - protein, 17 aa, synthetic construct
  REGION 1..17: note = CDR-H2
IIDPSDGNTNYAQNFQG

SEQ ID NO: 485 - protein, 13 aa, synthetic construct
  REGION 1..13: note = CDR-H3
ERAAAGYYYYMDV

SEQ ID NO: 486 - protein, 11 aa, synthetic construct
  REGION 1..11: note = CDR-L1
RASQSISSYLN

SEQ ID NO: 487 - protein, 7 aa, synthetic construct
  REGION 1..7: note = CDR-L2
AASSLQS

SEQ ID NO: 488 - protein, 9 aa, synthetic construct
  REGION 1..9: note = CDR-L3
QQSYSTPLT

SEQ ID NO: 489 - protein, 245 aa, synthetic construct
  REGION 1..245: note = CD8 scFv
QVQLVQSGAEVKKPGASVKVSCKASGGTFSSYAISWVRQAPGQGLEWMGIIDPSDGNTNYAQNFQGRVTMTRDTSTSTVYMELSSLRSEDTAVYYCAKERAAAGYYYYMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 490 - protein, 5 aa, synthetic construct
  REGION 1..5: note = CDR-H1
DYYIQ

SEQ ID NO: 491 - protein, 17 aa, synthetic construct
  REGION 1..17: note = CDR-H2
WINPNSGGTSYAQKFQG

SEQ ID NO: 492 - protein, 10 aa, synthetic construct
  REGION 1..10: note = CDR-H3
EGDYYYGMDA

SEQ ID NO: 493 - protein, 16 aa, synthetic construct
  REGION 1..16: note = CDR-L1
RSSQSLLHSNGYNYLD

SEQ ID NO: 494 - protein, 7 aa, synthetic construct
  REGION 1..7: note = CDR-L2
LGSNRAS

SEQ ID NO: 495 - protein, 9 aa, synthetic construct
  REGION 1..9: note = CDR-L3
MQGLQTPHT

SEQ ID NO: 496 - protein, 247 aa, synthetic construct
  REGION 1..247: note = CD8 scFv
QVQLVQSGAEVKKPGASVKVSCKASGYTFTDYYIQWVRQAPGQGLEWMGWINPNSGGTSYAQKFQGRVTMTRDTSTSTVYMELSSLRSEDTAVYYCAKEGDYYYGMDAWGQGTMVTVSSGGGGSGGGGSGGGGSDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTPHTFGQGTKVEIKR

SEQ ID NO: 497 - protein, 5 aa, synthetic construct
  REGION 1..5: note = CDR-H1
SYYMH

SEQ ID NO: 498 - protein, 17 aa, synthetic construct
  REGION 1..17: note = CDR-H2
GFDPEDGETIYAQKFQG

SEQ ID NO: 499 - protein, 8 aa, synthetic construct
  REGION 1..8: note = CDR-H3
DQGWGMDV

SEQ ID NO: 500 - protein, 9 aa, synthetic construct
  REGION 1..9: note = CDR-L3
QQTYSTPYT

SEQ ID NO: 501 - protein, 240 aa, synthetic construct
  REGION 1..240: note = CD8 scFv
QVQLVQSGAEVKKPGASVKVSCKASGYTFTSYYMHWVRQAPGQGLEWMGGFDPEDGETIYAQKFQGRVTMTRDTSTSTVYMELSSLRSEDTAVYYCARDQGWGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQTYSTPYTFGQGTKLEIKR

SEQ ID NO: 502 - protein, 5 aa, synthetic construct
  REGION 1..5: note = CDR-H1
NHYMH

SEQ ID NO: 503 - protein, 17 aa, synthetic construct
  REGION 1..17: note = CDR-H2
WMNPNSGNTGYAQKFQG

SEQ ID NO: 504 - protein, 9 aa, synthetic construct
  REGION 1..9: note = CDR-H3
SESGSDLDY

SEQ ID NO: 505 - protein, 11 aa, synthetic construct
  REGION 1..11: note = CDR-L1
RASQTIGNYVN

SEQ ID NO: 506 - protein, 7 aa, synthetic construct
  REGION 1..7: note = CDR-L2
GASNLHT

SEQ ID NO: 507 - protein, 9 aa, synthetic construct
  REGION 1..9: note = CDR-L3
QQTYSAPLT

SEQ ID NO: 508 - protein, 241 aa, synthetic construct
  REGION 1..241: note = CD8 scFv
QVQLVQSGAEVKKPGASVKVSCKASGYTFTNHYMHWVRQAPGQGLEWMGWMNPNSGNTGYAQKFQGRVTMTRDTSTSTVYMELSSLRSEDTAVYYCASSESGSDLDYWGQGTLVTVSSGGGGSGGGGSGGGGSDIQMTQSPSSLSASVGDRVTITCRASQTIGNYVNWYQQKPGKAPKLLIYGASNLHTGVPSRFSGSGSGTDFTLTISSLQPEDFATYYCQQTYSAPLTFGGGTKVEIKR

SEQ ID NO: 509 - protein, 5 aa, synthetic construct
  REGION 1..5: note = CDR-H1
GYVMG

SEQ ID NO: 510 - protein, 17 aa, synthetic construct
  REGION 1..17: note = CDR-H2
AISRGGLSTSYADSVKG

SEQ ID NO: 511 - protein, 16 aa, synthetic construct
  REGION 1..16: note = CDR-H3
DRSDLYEITAASNIDS

SEQ ID NO: 512 - protein, 127 aa, synthetic construct
  REGION 1..127: note = CD117 VHH (b12)
QVQLVESGGGLVQAGGSLRLSCAASGRTNSNYAMGWFRQAPGKEREFVSVIGRGLGSTYYADSVKGRFTISRDNAKNTVYLQMNSLKPEDAAVYYCAADLEFVFDEMGWIPGEHAYWGRGTLVTVSS

SEQ ID NO: 513 - protein, 125 aa, synthetic construct
  REGION 1..125: note = CD117 VHH (b34)
QVQLVESGGRLVQAGESLRLSCSASGGTFSSYLMAWFRQVPGKEREIVAFITWHGHTTYYTDSVKGRFAISRDNARNTMYLQMNSLRPEDTAVYYCAADQRQEFSGTYSREYNYWGQGTQVTVSS

SEQ ID NO: 514 - protein, 127 aa, synthetic construct
  REGION 1..127: note = CD117 VHH (b41)
QVQLVESGGGLVQPGGSLRLSCAASGQTFTDYHHHVAWFRQAPGRERDFVAVISWSTGNTDYADSQRARFTISKDNAKNTVYLQMNNLKFEDTAVYYCAARDYHPRFSYSADDYNYWGQGTLVTVSS

SEQ ID NO: 515 - protein, 121 aa, synthetic construct
  REGION 1..121: note = CD117 VHH (b46)
EVQLVESGGGLVQAGGSLRLSCAASGSSINEMGYYRQAPGKERAPRELVATITSGGSTNYADSVKGRFTISRDNAKNTVYLQMNSLKPEDTAVYYCAALRIMTMGRPYDYWGQGTQVTVSS

SEQ ID NO: 516 - protein, 246 aa, synthetic construct
  REGION 1..246: note = CD133 scFv (b3)
EIQLQQSGPDLMKPGASVKISCKASGYSFTNYYVHWVKQSLDKSLEWIGYVDPFNGDFNYNQKFKDKATLTVDKSSSTAYMHLSSLTSEDSAVYYCARGGLDWYDTSYWYFDVWGAGTAVTVSSGGGGSGGGGSGGGGSNIVMTQSPKSMSMSLGERVTLSCKASENVDTYVSWYQQKPEQSPKVLIYGASNRYTGVPDRFTGSGSATDFSLTISNVQAEDLADYHCGQSYRYPLTFGAGTKLELK

SEQ ID NO: 517 - protein, 124 aa, synthetic construct
  REGION 1..124: note = CD133 VH (b3)
EIQLQQSGPDLMKPGASVKISCKASGYSFTNYYVHWVKQSLDKSLEWIGYVDPFNGDFNYNQKFKDKATLTVDKSSSTAYMHLSSLTSEDSAVYYCARGGLDWYDTSYWYFDVWGAGTAVTVSS

SEQ ID NO: 518 - protein, 5 aa, synthetic construct
  REGION 1..5: note = CD133 CDR-H1 (b3) (Kabat)
NYYVH

SEQ ID NO: 519 - protein, 17 aa, synthetic construct
  REGION 1..17: note = CD133 CDR-H2 (b3) (Kabat)
YVDPFNGDFNYNQKFKD

SEQ ID NO: 520 - protein, 15 aa, synthetic construct
  REGION 1..15: note = CD133 CDR-H3 (b3) (Kabat, Chothia)
GGLDWYDTSYWYFDV

SEQ ID NO: 521 - protein, 107 aa, synthetic construct
  REGION 1..107: note = CD133 VL (b3)
NIVMTQSPKSMSMSLGERVTLSCKASENVDTYVSWYQQKPEQSPKVLIYGASNRYTGVPDRFTGSGSATDFSLTISNVQAEDLADYHCGQSYRYPLTFGAGTKLELK

SEQ ID NO: 522 - protein, 11 aa, synthetic construct
  REGION 1..11: note = CD133 CDR-L1 (b3) (Kabat, Chothia)
KASENVDTYVS

SEQ ID NO: 523 - protein, 7 aa, synthetic construct
  REGION 1..7: note = CD133 CDR-L2 (b3) (Kabat)
GASNRYT

SEQ ID NO: 524 - protein, 9 aa, synthetic construct
  REGION 1..9: note = CD133 CDR-L3 (b3) (Kabat)
GQSYRYPLT

SEQ ID NO: 525 - protein, 247 aa, synthetic construct
  REGION 1..247: note = CD133 scFv (b4)
EVQLVESGGGLVQPGGSLRLSCAASGFNIYYYGSMHWVRQAPGKGLEWVASISPYYGSTYYADSVKGRFTISADTSKNTAYLQMNSLRAEDTAVYYCARHASSGYGHYAVYGIDYWGQGTLVTVSSGGGGSGGGGSGGGGSDIQMTQSPSSLSASVGDRVTITCRASQGSSYVAWYQQKPGKAPKLLIYSASYLYSGVPSRFSGSRSGTDFTLTISSLQPEDFATYYCQQGVWSLITFGQGTKVEIK

SEQ ID NO: 526 - protein, 126 aa, synthetic construct
  REGION 1..126: note = CD133 VH (b4)
EVQLVESGGGLVQPGGSLRLSCAASGFNIYYYGSMHWVRQAPGKGLEWVASISPYYGSTYYADSVKGRFTISADTSKNTAYLQMNSLRAEDTAVYYCARHASSGYGHYAVYGIDYWGQGTLVTVSS

SEQ ID NO: 527 - protein, 6 aa, synthetic construct
  REGION 1..6: note = CD133 CDR-H1 (b4) (Kabat)
YYGSMH

SEQ ID NO: 528 - protein, 17 aa, synthetic construct
  REGION 1..17: note = CD133 CDR-H2 (b4) (Kabat)
SISPYYGSTYYADSVKG

SEQ ID NO: 529 - protein, 16 aa, synthetic construct
  REGION 1..16: note = CD133 CDR-H3 (b4) (Kabat, Chothia)
HASSGYGHYAVYGIDY

SEQ ID NO: 530 - protein, 106 aa, synthetic construct
  REGION 1..106: note = CD133 VL (b4)
DIQMTQSPSSLSASVGDRVTITCRASQGSSYVAWYQQKPGKAPKLLIYSASYLYSGVPSRFSGSRSGTDFTLTISSLQPEDFATYYCQQGVWSLITFGQGTKVEIK

SEQ ID NO: 531 - protein, 10 aa, synthetic construct
  REGION 1..10: note = CD133 CDR-L1 (b4) (Kabat, Chothia)
RASQGSSYVA

SEQ ID NO: 532 - protein, 7 aa, synthetic construct
  REGION 1..7: note = CD133 CDR-L1 (b4) (Kabat, Chothia)
SASYLYS

SEQ ID NO: 533 - protein, 9 aa, synthetic construct
  REGION 1..9: note = CD133 CDR-L1 (b4) (Kabat, Chothia)
QQGVWSLIT

SEQ ID NO: 534 - protein, 238 aa, synthetic construct
  REGION 1..238: note = CD133 scFv (b1)
EVKLVESGPELVKPGETVKISCKASGYTFTDYSMHWVRQAPGKGLEWMGWINTETGEPSYADDFKGRFAFSLDTSASTAYLQINSLRSEDTATYFCATDYGDYFDYWGQGTTVTVSSGGGGSGGGGSGGGGSDIVLSQSPAIMSASPGEKVTISCSASSSVSYMYWYQQKPGSSPKPWIYRTSNLASGVPARFSGSGSGTSYSLTISSMEPEDAATYYCQQYHSYPPTFGAGTKLELK

SEQ ID NO: 535 - protein, 117 aa, synthetic construct
  REGION 1..117: note = CD133 VH (b1)
EVKLVESGPELVKPGETVKISCKASGYTFTDYSMHWVRQAPGKGLEWMGWINTETGEPSYADDFKGRFAFSLDTSASTAYLQINSLRSEDTATYFCATDYGDYFDYWGQGTTVTVSS

SEQ ID NO: 536 - protein, 5 aa, synthetic construct
  REGION 1..5: note = CD133 CDR-H1 (b1) (Kabat)
DYSMH

SEQ ID NO: 537 - protein, 17 aa, synthetic construct
  REGION 1..17: note = CD133 CDR-H2 (b1) (Kabat)
WINTETGEPSYADDFKG

SEQ ID NO: 538 - protein, 8 aa, synthetic construct
  REGION 1..8: note = CD133 CDR-H3 (b1) (Kabat, Chothia)
DYGDYFDY

SEQ ID NO: 539 - protein, 106 aa, synthetic construct
  REGION 1..106: note = CD133 VL (b1)
DIVLSQSPAIMSASPGEKVTISCSASSSVSYMYWYQQKPGSSPKPWIYRTSNLASGVPARFSGSGSGTSYSLTISSMEPEDAATYYCQQYHSYPPTFGAGTKLELK

SEQ ID NO: 540 - protein, 10 aa, synthetic construct
  REGION 1..10: note = CD133 CDR-L1 (b1) (Kabat, Chothia)
SASSSVSYMY

SEQ ID NO: 541 - protein, 7 aa, synthetic construct
  REGION 1..7: note = CD133 CDR-L2 (b1) (Kabat, Chothia)
RTSNLAS

SEQ ID NO: 542 - protein, 9 aa, synthetic construct
  REGION 1..9: note = CD133 CDR-L3 (b1) (Kabat, Chothia)
QQYHSYPPT

SEQ ID NO: 543 - protein, 245 aa, synthetic construct
  REGION 1..245: note = CD133 scFv (b2)
DVQLQESGPGLVKPSQSLSLTCSVTGYSITSGYYWSWIRQFPGNKLEWMGYISYDGSNNYNPSLKNRISITRDTSKNQIFLKLNSVTTEDTATYYCTRVYFGHPYYYGMDYWGQGISVTVGGGGSGGGGSGGGGSQIVLTQSPAIMSASLGERVTMTCTASSSVSSFYLHWYRQKPGSSPKLWIYSTSNLASGVPARFSGSGSGTSYSLTISSMEAEDAATYYCHQYHRSPYMYMFGGGTKLEIK

SEQ ID NO: 544 - protein, 120 aa, synthetic construct
  REGION 1..120: note = CD133 VH (b2)
DVQLQESGPGLVKPSQSLSLTCSVTGYSITSGYYWSWIRQFPGNKLEWMGYISYDGSNNYNPSLKNRISITRDTSKNQIFLKLNSVTTEDTATYYCTRVYFGHPYYYGMDYWGQGISVTV

SEQ ID NO: 545 - protein, 6 aa, synthetic construct
  REGION 1..6: note = CD133 CDR-H1 (b2) (Kabat)
SGYYWS

SEQ ID NO: 546 - protein, 16 aa, synthetic construct
  REGION 1..16: note = CD133 CDR-H2 (b2) (Kabat)
YISYDGSNNYNPSLKN

SEQ ID NO: 547 - protein, 13 aa, synthetic construct
  REGION 1..13: note = CD133 CDR-H3 (b2) (Kabat, Chothia)
VYFGHPYYYGMDY

SEQ ID NO: 548 - protein, 110 aa, synthetic construct
  REGION 1..110: note = CD133 VL (b2)
QIVLTQSPAIMSASLGERVTMTCTASSSVSSFYLHWYRQKPGSSPKLWIYSTSNLASGVPARFSGSGSGTSYSLTISSMEAEDAATYYCHQYHRSPYMYMFGGGTKLEIK

SEQ ID NO: 549 - protein, 12 aa, synthetic construct
  REGION 1..12: note = CD133 CDR-L1 (b2) (Kabat, Chothia)
TASSSVSSFYLH

SEQ ID NO: 550 - protein, 7 aa, synthetic construct
  REGION 1..7: note = CD133 CDR-L2 (b2) (Kabat, Chothia)
STSNLAS

SEQ ID NO: 551 - protein, 11 aa, synthetic construct
  REGION 1..11: note = CD133 CDR-L3 (b2) (Kabat, Chothia)
HQYHRSPYMYM

SEQ ID NO: 552 - protein, 242 aa, synthetic construct
  REGION 1..242: note = CD133 scFv (b5)
EVQLVESGGGLVQPGGSLRLSCAASGFNLSSSSIHWVRQAPGKGLEWVAYIYPYYSYTYYADSVKGRFTISADTSKNTAYLQMNSLRAEDTAVYYCARFGSVAGFDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYSHAGHLFTFGQGTKVEIK

SEQ ID NO: 553 - protein, 118 aa, synthetic construct
  REGION 1..118: note = CD133 VH (b5)
EVQLVESGGGLVQPGGSLRLSCAASGFNLSSSSIHWVRQAPGKGLEWVAYIYPYYSYTYYADSVKGRFTISADTSKNTAYLQMNSLRAEDTAVYYCARFGSVAGFDYWGQGTLVTVSS

SEQ ID NO: 554 - protein, 5 aa, synthetic construct
  REGION 1..5: note = CD133 CDR-H1 (b5) (Kabat)
SSSIH

SEQ ID NO: 555 - protein, 17 aa, synthetic construct
  REGION 1..17: note = CD133 CDR-H2 (b5) (Kabat)
YIYPYYSYTYYADSVKG

SEQ ID NO: 556 - protein, 9 aa, synthetic construct
  REGION 1..9: note = CD133 CDR-H3 (b5) (Kabat, Chothia)
FGSVAGFDY

SEQ ID NO: 557 - protein, 109 aa, synthetic construct
  REGION 1..109: note = CD133 VL (b5)
DIQMTQSPSSLSASVGDRVTITCRASQSVSSAVAWYQQKPGKAPKLLIYSASSLYSGVPSRFSGSRSGTDFTLTISSLQPEDFATYYCQQYSHAGHLFTFGQGTKVEIK

SEQ ID NO: 558 - protein, 11 aa, synthetic construct
  REGION 1..11: note = CD133 CDR-L1 (b5) (Kabat, Chothia)
RASQSVSSAVA

SEQ ID NO: 559 - protein, 7 aa, synthetic construct
  REGION 1..7: note = CD133 CDR-L2 (b5) (Kabat, Chothia)
SASSLYS

SEQ ID NO: 560 - protein, 11 aa, synthetic construct
  REGION 1..11: note = CD133 CDR-L3 (b5) (Kabat, Chothia)
QQYSHAGHLFT

SEQ ID NO: 561 - protein, 604 aa, synthetic construct
  REGION 1..604: note = Henipavirus protein G (GENBANK ID: AF017149)
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 562 - protein, 622 aa, synthetic construct
  REGION 1..622: note = Henipavirus protein G (GENBANK ID: JQ001776)
MLSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 563 - protein, 632 aa, synthetic construct
  REGION 1..632: note = Henipavirus protein G (GENBANK ID: NC_025256)
MPQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 564 - protein, 625 aa, synthetic construct
  REGION 1..625: note = Henipavirus protein G (GENBANK ID: NC_025352)
MATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 565 - protein, 6 aa, synthetic construct
  REGION 1..6: note = CD133 CDR-H2 (b1) (Chothia)
NTETGE

SEQ ID NO: 566 - protein, 8 aa, synthetic construct
  REGION 1..8: note = CD133 CDR-H1 (b2) (Chothia)
GYSITSGY

SEQ ID NO: 567 - protein, 5 aa, synthetic construct
  REGION 1..5: note = CD133 CDR-H2 (b2) (Chothia)
SYDGS

SEQ ID NO: 568 - protein, 7 aa, synthetic construct
  REGION 1..7: note = CD133 CDR-H1 (b3) (Chothia)
GYSFTNY

SEQ ID NO: 569 - protein, 6 aa, synthetic construct
  REGION 1..6: note = CD133 CDR-H2 (b3) (Chothia)
DPFNGD

SEQ ID NO: 570 - protein, 8 aa, synthetic construct
  REGION 1..8: note = CD133 CDR-H1 (b4) (Chothia)
GFNIYYYG

SEQ ID NO: 571 - protein, 6 aa, synthetic construct
  REGION 1..6: note = CD133 CDR-H2 (b4) (Chothia)
SPYYGS

SEQ ID NO: 572 - protein, 7 aa, synthetic construct
  REGION 1..7: note = CD133 CDR-H1 (b5) (Chothia)
GFNLSSS

SEQ ID NO: 573 - protein, 6 aa, synthetic construct
  REGION 1..6: note = CD133 CDR-H2 (b5) (Chothia)
YPYYSY

SEQ ID NO: 574 - protein, 822 aa, synthetic construct
  REGION 1..822: note = Blinded NiVG protein attachment glycoprotein + linker + CD133 scFv (b1)
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSEVKLVESGPELVKPGETVKISCKASGYTFTDYSMHWVRQAPGKGLEWMGWINTETGEPSYADDFKGRFAFSLDTSASTAYLQINSLRSEDTATYFCATDYGDYFDYWGQGTTVTVSSGGGGSGGGGSGGGGSDIVLSQSPAIMSASPGEKVTISCSASSSVSYMYWYQQKPGSSPKPWIYRTSNLASGVPARFSGSGSGTSYSLTISSMEPEDAATYYCQQYHSYPPTFGAGTKLELK

SEQ ID NO: 575 - protein, 127 aa, synthetic construct
  REGION 1..127: note = CD117 VHH (b1)
EVQLVESGGGLVQAGGSLRLSCKASGRTNSNYAMGWFRQAPGKEREFVTVIGRSIGMTYYADSVKGRFTISRDNAKNTVYLQMNSLKPEDTAVYYCAADLEFVFGETGWIPGDDAYWGQGTQVTVSS

SEQ ID NO: 576 - protein, 127 aa, synthetic construct
  REGION 1..127: note = CD117 VHH (b2)
QVQLVESGGGLVQAGGSLRLSCAASGRTNSNYAMGWFRQAPGKEREFVSVIGRGLGSTYYADSVKGRFTISRDNAKNTVYLQVNSLKPEDTAVYYCAADLEFVFDEMGWIPGEHAYWGRGTLVTVSS

SEQ ID NO: 577 - protein, 127 aa, synthetic construct
  REGION 1..127: note = CD117 VHH (b3)
EVQLVESGGGLVQPGGSLRLSCTASGRTNSNYAMGWFRQAPGKEREFVSVIGRGLGSTYYADSVKGRFTISRDNAKNTVYLQMNSLKPEDTAVYYCAADLEFVFDETSWIPGEHAYWGRGTLVTVSS

SEQ ID NO: 578 - protein, 127 aa, synthetic construct
  REGION 1..127: note = CD117 VHH (b4)
EVQLVESGGGLVQAGGSLRLSCAASGRTNSNYAMGWFRQAPGKEREFVTVIGRSIGMTYYADSVKGRFTISRDNAKNTVYLQMNSLKPEDTAVYYCAADLEFVFDETGWIPGDDAYWGQGTLVTVSS

SEQ ID NO: 579 - protein, 127 aa, synthetic construct
  REGION 1..127: note = CD117 VHH (b5)
QVQLVESGGGLVQAGGSLRLSCAASGRTNSNYAMGWFRQAPGKEREFVSVIGRGLGSTYYADSVKGRFTISRDNAKNTVYLQMNSLKPEDTAVYYCAADLEFVFDGMGWIPGEHAYWGRGTLVTVSS

SEQ ID NO: 580 - protein, 127 aa, synthetic construct
  REGION 1..127: note = CD117 VHH (b6)
EVQLVESGGGLVQAGGSLRLSCAASGRTNSNYAMGWFRQAPGKEREFVSVIGRGLGSTYYADSVKGRFTISRDNAKNTVYLQMNSLKPEDTAVYYCAADLEFVFDEMGWIPGEHAYWGRGTLVTVSS

SEQ ID NO: 581 - protein, 127 aa, synthetic construct
  REGION 1..127: note = CD117 VHH (b7)
EVQLVESGGRLVRAGGSLRLSCAASGRTNSNYAMGWFRQAPGKEREFVSVIGRGLGSTYYADSVKGRFTISRDNAKNTVYLQMNSLKPEDTAVYFCAADLEFVFDEMGWIPGEHAYWGRGTLVTVSS

SEQ ID NO: 582 - protein, 127 aa, synthetic construct
  REGION 1..127: note = CD117 VHH (b8)
EVQLVESGGGLVQAGGSLRLSCAASGRTNSNYAMGWFRQAPGKEREFVSVIGRGLGSTYYADSVKGRFTISRDNAKNTVYLQMNSLKPEDTSVYYCAADLEFVFDEMGWIPGEHAYWGRGTQVTVSS

SEQ ID NO: 583 - protein, 127 aa, synthetic construct
  REGION 1..127: note = CD117 VHH (b9)
QVQLVESGGGLVQAGGSLGLSCAASGRTFSRMMMGWFRQAPGKEREFVSVIGRGLGSTYYADSVKGRFTISRDNAKNTVYLQMNSLKPEDTAVYYCAADLEFVFDEMGWIPGEHAYWGRGTQVTVSS

SEQ ID NO: 584 - protein, 127 aa, synthetic construct
  REGION 1..127: note = CD117 VHH (b10)
QVQLVESGGGLVQAGGSLRLSCAASGRTNSNYAMGWFRQAPGKEREFVSVIGRGLGSTYYADSVKGRFTISRDNAKNTVYLQMNSLKPEDTAVYYCAADLEFVFDEMGWIPGEHAYWGRGTQVTVSS

SEQ ID NO: 585 - protein, 127 aa, synthetic construct
  REGION 1..127: note = CD117 VHH (b11)
QVQLVESGGGLVQAGGSLRLSCAASGRTNSNYAMGWFRQAPGKEREFVSVIGRGLGSTYCADSVKGRFTTSRDNAKNTVYLQMNSLKPEDTAVYYCAADLEFVFDEMGWIPGEHAYWGRGTQVTVSS

SEQ ID NO: 586 - protein, 127 aa, synthetic construct
  REGION 1..127: note = CD117 VHH (b13)
EVQLVESGGGLVQAGGSLRLSCAASGRTNSNYAMGWFRQAPGKEREFVSVIGRGLGSTYYADSVKGRFTISRDNAKNTVYLQMNSLKPEDTAVYYCAADLEFVFDEMGWIPGEHAYWGRGTQVTVSS

SEQ ID NO: 587 - protein, 127 aa, synthetic construct
  REGION 1..127: note = CD117 VHH (b14)
EVQLVESGGGLVQAGGSLRLSCAASGRTNSDYAMGWFRQAPGKEREFVSVIGRGLGSTYYADSVKGRFTISRDNAKNTVYLQMNSLKPEDTAVYYCAADLEFVFDEMGWIPGEHAYWGRGTQVTVSS

SEQ ID NO: 588 - protein, 127 aa, synthetic construct
  REGION 1..127: note = CD117 VHH (b15)
EVQLVESGGGLVQAGGSLRLSCAASGRTNSNYAMGWFRQAPGKEREFVSVIGRGLGSTYYADSVKGRFTISRDNAKNTVYLQMNSLKPEDTAVYYCVADLEFVFDEMGWIPGEHAYWGRGTLVTVSS

SEQ ID NO: 589 - protein, 122 aa, synthetic construct
  REGION 1..122: note = CD117 VHH (b16)
EVQLVESGGGLAQAGDSLRLSCAASGRFDTSYAMGWFRQAPGKEREFVAGILSGTVKAYEDSVRGRFSISRDKAKNTVYLQMNSLKSEDTAVYYCVADVSFGDLTNGRSDYWGQGTQVTVSS

SEQ ID NO: 590 - protein, 122 aa, synthetic construct
  REGION 1..122: note = CD117 VHH (b17)
EVQLVESGGGLAQAGDSLRLSCAASGRFDTSYAMGWFRQAPGKEREFVAGILSGTVKAYEDSVRGRFSISRDKAKNTVYLQMNSLKPEDTAVYYCVADVSFGDLTNGRSDYWGQGTQVTVSS

SEQ ID NO: 591 - protein, 122 aa, synthetic construct
  REGION 1..122: note = CD117 VHH (b18)
EVQLVESGGGLAQAGDSLRLSCAASGRFDTSYAMGWFRQAPGKEREFVAGILSGTVKAYEDSVRGRFSISRDKAKNTVYLQMNSLKPEDTAVYYCVADVSFGDLTNGRSDYWGQGTLVTVSS

SEQ ID NO: 592 - protein, 122 aa, synthetic construct
  REGION 1..122: note = CD117 VHH (b19)
QVQLVESGGGLAQAGDSLRLSCAASGRFDTSYAMGWFRQAPGKEREFVAGILSGTVKAYEDSVRGRFSISRDKAKNTVYLQMNSLKSEDTAVYYCVADVSFGDLTNGRSDYWGQGTQVTVSS

SEQ ID NO: 593 - protein, 122 aa, synthetic construct
  REGION 1..122: note = CD117 VHH (b20)
EVQLVESGGGLAQAGDSLRLSCAASGRFDTSYAMGWFRQAPGKEREFVAGILSGTVKAYEDSVRGRFSISRDKAKNTVYLQMNSLKSEDTAVYYCVADVSFGDLTNGRSDYWGQGTLVTVSS

SEQ ID NO: 594 - protein, 122 aa, synthetic construct
  REGION 1..122: note = CD117 VHH (b21)
EVQLVESGGGLAQAGDSLRLSCAASGRFDTSYAMGWFRQAPGKEREFVAGILSGAVKAYEDSVRGRFSISRDKAKNTVYLQMNSLKPEDTAVYYCVADVSFGDLTNGRSDYWGQGTQVTVSS

SEQ ID NO: 595 - protein, 122 aa, synthetic construct
  REGION 1..122: note = CD117 VHH (b22)
QVQLVESGGGLAQAGDSLRLSCAASGRFDTSYAMGWFRQAPGKEREFVAGILSGTVKAYEDSVRGRFSISRDKAKNTVYLQMNSLKPEDTAVYYCVADVSFGDLTNGRSDYWGQGTQVTVSS

SEQ ID NO: 596 - protein, 122 aa, synthetic construct
  REGION 1..122: note = CD117 VHH (b23)
EVQLVESGGGLAQAGDSLRLSCAASGRFDASYAMGWFRQAPGKEREFVAGILSGTVKAYEDSVRGRFSISRDKAKNTVYLQMNSLKSEDTAVYYCVADVSFGDLTNGRSDYWGQGTLVTVSS

SEQ ID NO: 597 - protein, 122 aa, synthetic construct
  REGION 1..122: note = CD117 VHH (b24)
EVQLVESGGGLAQAGDSLRLSCAASGRFDTSYAMGWFRQAPGKEREFVAGILSGTVKAYEDSVRGRFSISRDQAKNTVYLQMNSLKPEDTAVYYCVADVSFGDLTNGRSDYWGQGTQVTVSS

SEQ ID NO: 598 - protein, 122 aa, synthetic construct
  REGION 1..122: note = CD117 VHH (b25)
EVQLVESGGGLAQAGDSLRLSCAASGRFDTSYAMGWFRQVPGKERGFVAGILSGTVKAYEDSVRGRFSISRDKAKNTVYLRMNSLKPEDTAVYYCVADVSFGDLTNGRSDYWGQGTQVTVSS

SEQ ID NO: 599 - protein, 126 aa, synthetic construct
  REGION 1..126: note = CD117 VHH (b26)
EVQLVESGGGLVRAGDSLRLSCAASEGRTFTMRSVGWFRQAPGKEREFVAASNWWSSGSTMYAEYVKGRFTISRDNAKNTVYLQMNSLKPEDTATYYCAASSTVYMSSRLSEYAYWGQGTQVTVSS

SEQ ID NO: 600 - protein, 126 aa, synthetic construct
  REGION 1..126: note = CD117 VHH (b27)
QVQLVESGGGLVRAGDSLRLSCAASEGRTFTMRSVGWFRQAPGKEREFVAASNWWSSGSTMYAEYVKGRFTISRDNAKNTVYLQMNSLKPEDTATYYCAASSTVYMSSRLSEYAYWGQGTQVTVSS

SEQ ID NO: 601 - protein, 124 aa, synthetic construct
  REGION 1..124: note = CD117 VHH (b28)
EVQLVESGGGLVQTGGSLTLSCAASKRTLNTYAMGWFRQPPGKRREFVAADSWSGGSTMYAEYVKGRFTISRDNAKNTVYLQMNSLKPEDTATYYCAASSTVYMSSRLSEYAYWGQGTQVTVSS

SEQ ID NO: 602 - protein, 124 aa, synthetic construct
  REGION 1..124: note = CD117 VHH (b29)
EVQLVESGGGWVQTDDSLRLSCAASGGAFGNYAMGWLRQAPGKGREFVAGIDWSGGSTMYAEYVKGRFTISRDNAKNTVYLQMNSLKPEDTATYYCAASSTVYMSSRLSEYAYWGQGTLVTVSS

SEQ ID NO: 603 - protein, 124 aa, synthetic construct
  REGION 1..124: note = CD117 VHH (b30)
QVQLVESGGGWVQTDDSLRLSCAASGGAFGNYAMGWLRQAPGKGREFVAGIDWSGGSTMYAEYVKGRFTISRDNAKNTVYLQMNSLKPEDTATYYCAASSTVYMSSRLSEYAYWGQGTQVTVSS

SEQ ID NO: 604 - protein, 124 aa, synthetic construct
  REGION 1..124: note = CD117 VHH (b31)
QVQLVESGGGSVQTGDSLRLSCVASGSTFGSYAMAWYRQVPGKEREFVAAVRWSGGSTMYAEYVKGRFTISRDNAKNTVYLQMNGLKPEDTATYYCAASSTVYMSSRLSEYAYWGQGTLVTVSS

SEQ ID NO: 605 - protein, 125 aa, synthetic construct
  REGION 1..125: note = CD117 VHH (b32)
EVQLVESGGRLVQAGESLRLSCSASGGTFSSYLMAWFRQVPGKEREIVAFITWHGHTTYYTDSVKGRFAISRDNARNTMYLQMNSLRPEDTAVYYCAADQRQEFSGTYSREYNYWGQGTLVTVSS

SEQ ID NO: 606 - protein, 125 aa, synthetic construct
  REGION 1..125: note = CD117 VHH (b33)
QVQLVESGGRLVQAGESLRLSCSASGGTFSSYLMAWFRQVPGKEREIVAFITWHGHTTYYTDSVKGRFAISRDNARNTMYLQMNSLRPEDTAVYYCAADQRQEFSGTYSREYNYWGQGTLVTVSS

SEQ ID NO: 607 - protein, 125 aa, synthetic construct
  REGION 1..125: note = CD117 VHH (b35)
EVQLVESGGGLVQAGGSLGLACVASGDTLSTYFMGWFRQAPGKEREFVAAIGWSGWLTYYADSMKGRFTISRDNGKNSVYLQMNNLKRDDTAVYYCAAGKTFAAAPSTTLIGDYWGQGTLVTVSS

SEQ ID NO: 608 - protein, 125 aa, synthetic construct
  REGION 1..125: note = CD117 VHH (b36)
EVQLVESGGGLVQAGGSLRLACVASGDTLSTYFMGWFRQAPGKEREFVAAIGWSGWLTYYADSMKGRFTISRDNGKNSVYLQMNNLKRDDTAVYYCAAGKTFAAAPSTTLIGDYWGQGTQVTVSS

SEQ ID NO: 609 - protein, 125 aa, synthetic construct
  REGION 1..125: note = CD117 VHH (b37)
QVQLVESGGGLVQAGGSLRLSCVASGDTLSTYFMGWFRQAPGKEREFVAAIGWSGWLTYYADSMKGRFTISRDNGKNSVYLQMNNLKRDDTAVYYCAAGKTFAAAPSTTLIGDYWGQGTLVTVSS

SEQ ID NO: 610 - protein, 126 aa, synthetic construct
  REGION 1..126: note = CD117 VHH (b38)
EVQLVESGGGLVQVGGSLRLSCAASGRTTGRYFMGWFREAPGKEREFVSAIGWSVGSTYYVDSVKGRFTISRDNAKNTVYLQMNSLKPEDTAVYYCAVGFEFSQGAGTAELIGDYWGQGTQVTVSS

SEQ ID NO: 611 - protein, 126 aa, synthetic construct
  REGION 1..126: note = CD117 VHH (b39)
EVQLVESGGGLVQVGGSLRLSCAASGRTTGRYFMGWFREAPGKEREFVSAIGWSVGSTYYVDSVKGRFTISRDNANNTVYLQMNSLKPEDTAVYYCAVGFEFSQGAGTAELIGDYWGQGTQVTVSS

SEQ ID NO: 612 - protein, 126 aa, synthetic construct
  REGION 1..126: note = CD117 VHH (b40)
QVQLVESGGGLVQAGGSLTLSCAAPGRTFSNYFMGWFRQAPGKERDFVAAISWSGFLTYYADSVKGRFTISRDNAKNTVDLQMNRLKPEDTAVYYCAMGSEFSPGAGTAELIGDYWGQGTLVTVSS

SEQ ID NO: 613 - protein, 127 aa, synthetic construct
  REGION 1..127: note = CD117 VHH (b42)
QVQLVESGGGLVQPGGSLRLSCAASGQTFTDYHHHVAWFRQAPGRERDFVAVISWSTGNTDYADSQRARFTISKDNAKNTAYLQMNNLKFEDTAVYYCAARDYHPRFSYSADDYNYWGQGTQVTVSS

SEQ ID NO: 614 - protein, 131 aa, synthetic construct
  REGION 1..131: note = CD117 VHH (b43)
EVQLVESGGGLVQAGDSLRLSCTASISTSGNYATFKNYAMGWFRQAPGKAREFVAGISNSGGTTDYADSVKGRFTISRDNARKTTYLQMNRLGPEDTAVYSCAADAWFGPPKRDPTTYSFRGPGTLVTVSS

SEQ ID NO: 615 - protein, 124 aa, synthetic construct
  REGION 1..124: note = CD117 VHH (b44)
EVQLVESGGGLVQAGDSLRLSCAVSGRTFSSVAMAWLRQGPGKAREFVASISWSGSTTDYADSVKGRFTISRDNAKKMVYLQMNSLKPEDTAVYYCAAGRVPGSSPTIHNYVTWGQGTQVTVSS

SEQ ID NO: 616 - protein, 132 aa, synthetic construct
  REGION 1..132: note = CD117 VHH (b45)
EVQLVESGGGSAHTGGPLRLSCVGTGPTTGHTFRGYAVAWFRQAPGKEREFVAAVRWNEGQDVYYADSVKGRFTISRDNDKNTVYLQMNSLKPEDTAVYYCAAGVKWGSITFMASSSRYDFWGQGTQVTVSS

SEQ ID NO: 617 - protein, 119 aa, synthetic construct
  REGION 1..119: note = CD117 VHH (b47)
EVQLVESGGGLVQAGGSLRLSCAASGRTFSYYIMGWFRQAPGKEREFVAAINRSGGTTYYADSVEGRFFISRDNAKNTMYLQMNSLKPEDTAVYYCAAQPDSRGTYGYWGQGTLVTVSS

SEQ ID NO: 618 - protein, 125 aa, synthetic construct
  REGION 1..125: note = CD117 VHH (b48)
EVQLVESGGGLVQAGGSLRLSCIDSGRTFSRYYMGWFRQAPGKEREFVAAISQNGYTKWYAESVKGRSAISRDIAKNTVDLQMNSLKPEDTAVYYCAATSILEFSRDTASDFEYYGQGTLVTVSS

SEQ ID NO: 619 - protein, 122 aa, synthetic construct
  REGION 1..122: note = CD117 VHH (b49)
EVQLVESGGGLVRAGDSLRLSCSASGRTFSKWTMAWFRQAPGKEREFVAGMSGDATYYAASVKGRFTSSRDNSKNTVYLQMDSLKPEDTAVYFCAIYYGSGSYFQQNSYDTWGQGTQVTVSS

SEQ ID NO: 620 - protein, 123 aa, synthetic construct
  REGION 1..123: note = CD117 VHH (b50)
QVQLVESGGGLVQAGGSLRLSCTASGRTESTYFMSWFRQTPGKERDFVAVINYSGDVTTYADSVKGRFTISRDNAKNTVHLQMNSLTVEDTAVYYCAVHRTGPMNRADSYDYWGQGTLVTVSS

SEQ ID NO: 621 - protein, 125 aa, synthetic construct
  REGION 1..125: note = CD117 VHH (b51)
EVQLVESGGGLVQAGGSLKLSCAASGNIANTYRMAWYGQAPGKQRELVANITSDGIASYADSVKGRFTISRDNAKNTVYLQMNSLKAEDTAVYYCNIVYRRCSRGGYGCYSEYSWGQGTLVTVSS

SEQ ID NO: 622 - protein, 545 aa, synthetic construct
  REGION 1..545: note = BaEV envelope glycoprotein
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEEAQD

SEQ ID NO: 623 - protein, 17 aa, synthetic construct
  REGION 1..17: note = partial fusion inhibitory R peptide
VLTQQYQVLRTDEEAQD

SEQ ID NO: 624 - protein, 25 aa, synthetic construct
  REGION 1..25: note = BaEV Cytoplasmic tail
NRLTAFINDKLNIIHAMVLTQQYQV

SEQ ID NO: 625 - protein, 536 aa, synthetic construct
  REGION 1..536: note = Truncated BaEV envelope glycoprotein
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQV

SEQ ID NO: 626 - DNA, 1092 bp, synthetic construct
  source 1..1092: mol_type = other DNA
  misc_feature 1..1092: note = HA-CD3.s.001.01-IgG1ser-PDGFR
atggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgagcccaagtctagcgacaaaacccacacaagtccaccttcccctgccccagaagctgcaggcggacccaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 627 - DNA, 1032 bp, synthetic construct
  source 1..1032: mol_type = other DNA
  misc_feature 1..1032: note = HA-CD3.s.001.01-IgG1ser-GPI
atggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgagcccaagtctagcgacaaaacccacacaagtccaccttcccctgccccagaagctgcaggcggaccccagcttgagaacgggggcacatctctctccgaaaagactgtcctgttactggtgacccctttcttggctgccgcatggagcctgcacccctgataa

SEQ ID NO: 628 - DNA, 1089 bp, synthetic construct
  source 1..1089: mol_type = other DNA
  misc_feature 1..1089: note = HA-CD3.s.001.01-IgG1ser-VSVGan
atggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgagcccaagtctagcgacaaaacccacacaagtccaccttcccctgccccagaagctgcaggcggaccctcctctatcgccagcttcttcttcatcatcgggctgattatcggcctgttcctggtgctgagagtgggcatccacctgtgcatcaagctgaagcacaccaagaagcggcagatctacaccgacatcgagatgaaccggctgggcaaatgataa

SEQ ID NO: 629 - DNA, 1368 bp, synthetic construct
  source 1..1368: mol_type = other DNA
  misc_feature 1..1368: note = HA-CD3.s.001.01-
atggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgagcccaagtctagcgacaaaacccacacaagtccaccttcccctgccccagaagctgcaggcggacccattgcaagtcaaccactttctttacgacccgaggcctgtaggcccgctgctgggggtgctgttcataccaggggcctggacttcgcctgcgacatatacatttgggcccctcttgcaggcacttgtggagtgctgttgttatccctggtcatcaccctatattgtaatcaccgtaatcgccggcgagtgtgtaaatgccctcggccagtggttatcgctagccagccgctcagcctgaggccagaagcgtgccgccctgcagcaggaggggccgtccatacacggggcctcgattttgcctgtgatatttatatctgggcccccctcgcggggacgtgcggtgtcctgttgctgtcactggtgatcacactctactgcaaccacagaaacaggcgcagagtgtgtaagtgccctagacccgtagtgtgataa

SEQ ID NO: 630 - DNA, 1044 bp, synthetic construct
  source 1..1044: mol_type = other DNA
  misc_feature 1..1040: note = HA-CD3.s.001.01-IgG1ser-ICAM1
atggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgagcccaagtctagcgacaaaacccacacaagtccaccttcccctgccccagaagctgcaggcggacccatagtgattatcacagttgtcgccgccgctgtgattatgggaaccgcaggcctgagcacttatttgtacaaccggcagagaaagatcaagaaatacaggctctgataa

SEQ ID NO: 631 - DNA, 1059 bp, synthetic construct
  source 1..1059: mol_type = other DNA
  misc_feature 1..1059: note = HA-CD3.s.001.01-IgG1ser-HLADRB
atggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgagcccaagtctagcgacaaaacccacacaagtccaccttcccctgccccagaagctgcaggcggacccatgctgtctggtgtcggaggttttgtgctcggactcttgtttctgggggccggccttttcatctacttcaggaaccagaagggccacagcgggttacaacccaccggcttcctgtcctgataa

SEQ ID NO: 632 - DNA, 1044 bp, synthetic construct
  source 1..1044: mol_type = other DNA
  misc_feature 1..1044: note = HA-CD3.s.001.01-IgG1ser-CD49d
atggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgagcccaagtctagcgacaaaacccacacaagtccaccttcccctgccccagaagctgcaggcggacccatagtgatcatcagctccagtctgctgttagggctcattgttttgcttctgatttcttatgtcatgtggaaagccggctttttcaagaggcagtacaagtcatgataa

SEQ ID NO: 633 - DNA, 1140 bp, synthetic construct
  source 1..1140: mol_type = other DNA
  misc_feature 1..1140: note = TFR1-IgG1ser-CD3.s.001.01-HA
atgatggaccaagcccgcagcgcctttagtaatctgttcggcggggaacccttatcctataccaggttcagcttggctagacaggttgatggcgacaactcacacgtcgaaatgaaactcgccgtggacgaggaggagaatgcagataataacacaaaggcgaacgtgacgaaacctaagcggtgttccggatctatttgctacggtactatagctgtaattgtgttctttcttatcggatttatgatcggctacctgggggagcccaagtctagcgacaaaacccacacaagtccaccttcccctgccccagaagctgcaggcggacccgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccggaggaagttacccctacgatgtgcctgactatgcctgataa

SEQ ID NO: 634 - DNA, 1074 bp, synthetic construct
  source 1..1074: mol_type = other DNA
  misc_feature 1..1074: note = HA-CD3.s.001.01-EAAAKx3-PDGFR
atggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgcggaggctgctgctaaagaggccgccgccaaggaagcagcagccaaggcaaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 635 - DNA, 1014 bp, synthetic construct
  source 1..1014: mol_type = other DNA
  misc_feature 1..1014: note = HA-CD3.s.001.01-EAAAKx3-GPI
atggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgcggaggctgctgctaaagaggccgccgccaaggaagcagcagccaaggcacagcttgagaacgggggcacatctctctccgaaaagactgtcctgttactggtgacccctttcttggctgccgcatggagcctgcacccctgataa

SEQ ID NO: 636 - DNA, 1071 bp, synthetic construct
  source 1..1071: mol_type = other DNA
  misc_feature 1..1071: note = HA-CD3.s.001.01-EAAAKx3-VSVGan
atggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgcggaggctgctgctaaagaggccgccgccaaggaagcagcagccaaggcatcctctatcgccagcttcttcttcatcatcgggctgattatcggcctgttcctggtgctgagagtgggcatccacctgtgcatcaagctgaagcacaccaagaagcggcagatctacaccgacatcgagatgaaccggctgggcaaatgataa

SEQ ID NO: 637 - DNA, 1350 bp, synthetic construct
  source 1..1350: mol_type = other DNA
  misc_feature 1..1350: note = HA-CD3.s.001.01-EAAAKx3-CD8aTm
atggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgcggaggctgctgctaaagaggccgccgccaaggaagcagcagccaaggcaattgcaagtcaaccactttctttacgacccgaggcctgtaggcccgctgctgggggtgctgttcataccaggggcctggacttcgcctgcgacatatacatttgggcccctcttgcaggcacttgtggagtgctgttgttatccctggtcatcaccctatattgtaatcaccgtaatcgccggcgagtgtgtaaatgccctcggccagtggttatcgctagccagccgctcagcctgaggccagaagcgtgccgccctgcagcaggaggggccgtccatacacggggcctcgattttgcctgtgatatttatatctgggcccccctcgcggggacgtgcggtgtcctgttgctgtcactggtgatcacactctactgcaaccacagaaacaggcgcagagtgtgtaagtgccctagacccgtagtgtgataa

SEQ ID NO: 638 - DNA, 1041 bp, synthetic construct
  source 1..1041: mol_type = other DNA
  misc_feature 1..1041: note = HA-CD3.s.001.01-EAAAKx3-HLADRB
atggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgcggaggctgctgctaaagaggccgccgccaaggaagcagcagccaaggcaatgctgtctggtgtcggaggttttgtgctcggactcttgtttctgggggccggccttttcatctacttcaggaaccagaagggccacagcgggttacaacccaccggcttcctgtcctgataa

SEQ ID NO: 639 - DNA, 1026 bp, synthetic construct
  source 1..1026: mol_type = other DNA
  misc_feature 1..1026: note = HA-CD3.s.001.01-EAAAKx3-CD49d
atggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgcggaggctgctgctaaagaggccgccgccaaggaagcagcagccaaggcaatagtgatcatcagctccagtctgctgttagggctcattgttttgcttctgatttcttatgtcatgtggaaagccggctttttcaagaggcagtacaagtcatgataa

SEQ ID NO: 640 - DNA, 1122 bp, synthetic construct
  source 1..1122: mol_type = other DNA
  misc_feature 1..1122: note = TFR1-EAAAKx3-CD3.s.001.01-HA
atgatggaccaagcccgcagcgcctttagtaatctgttcggcggggaacccttatcctataccaggttcagcttggctagacaggttgatggcgacaactcacacgtcgaaatgaaactcgccgtggacgaggaggagaatgcagataataacacaaaggcgaacgtgacgaaacctaagcggtgttccggatctatttgctacggtactatagctgtaattgtgttctttcttatcggatttatgatcggctacctgggggcggaggctgctgctaaagaggccgccgccaaggaagcagcagccaaggcagcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccggaggaagttacccctacgatgtgcctgactatgcctgataa

SEQ ID NO: 641 - DNA, 1050 bp, synthetic construct
  source 1..1050: mol_type = other DNA
  misc_feature 1..1050: note = HA-CD3.s.004.01-IgG1ser-PDGFR
atggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggagcccaagtctagcgacaaaacccacacaagtccaccttcccctgccccagaagctgcaggcggacccaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 642 - DNA, 990 bp, synthetic construct
  source 1..990: mol_type = other DNA
  misc_feature 1..990: note = HA-CD3.s.004.01-IgG1ser-GPI
atggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggagcccaagtctagcgacaaaacccacacaagtccaccttcccctgccccagaagctgcaggcggaccccagcttgagaacgggggcacatctctctccgaaaagactgtcctgttactggtgacccctttcttggctgccgcatggagcctgcacccctgataa

SEQ ID NO: 643 - DNA, 1047 bp, synthetic construct
  source 1..1047: mol_type = other DNA
  misc_feature 1..1047: note = HA-CD3.s.004.01-IgG1ser-VSVGan
atggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggagcccaagtctagcgacaaaacccacacaagtccaccttcccctgccccagaagctgcaggcggaccctcctctatcgccagcttcttcttcatcatcgggctgattatcggcctgttcctggtgctgagagtgggcatccacctgtgcatcaagctgaagcacaccaagaagcggcagatctacaccgacatcgagatgaaccggctgggcaaatgataa

SEQ ID NO: 644 - DNA, 1002 bp, synthetic construct
  source 1..1002: mol_type = other DNA
  misc_feature 1..1002: note = HA-CD3.s.004.01-IgG1ser-ICAM1
atggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggagcccaagtctagcgacaaaacccacacaagtccaccttcccctgccccagaagctgcaggcggacccatagtgattatcacagttgtcgccgccgctgtgattatgggaaccgcaggcctgagcacttatttgtacaaccggcagagaaagatcaagaaatacaggctctgataa

SEQ ID NO: 645 - DNA, 1017 bp, synthetic construct
  source 1..1017: mol_type = other DNA
  misc_feature 1..1017: note = HA-CD3.s.004.01-IgG1ser-HLADRB
atggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggagcccaagtctagcgacaaaacccacacaagtccaccttcccctgccccagaagctgcaggcggacccatgctgtctggtgtcggaggttttgtgctcggactcttgtttctgggggccggccttttcatctacttcaggaaccagaagggccacagcgggttacaacccaccggcttcctgtcctgataa

SEQ ID NO: 646 - DNA, 1002 bp, synthetic construct
  source 1..1002: mol_type = other DNA
  misc_feature 1..1002: note = HA-CD3.s.004.01-IgG1ser-CD49d
atggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggagcccaagtctagcgacaaaacccacacaagtccaccttcccctgccccagaagctgcaggcggacccatagtgatcatcagctccagtctgctgttagggctcattgttttgcttctgatttcttatgtcatgtggaaagccggctttttcaagaggcagtacaagtcatgataa

SEQ ID NO: 647 - DNA, 1098 bp, synthetic construct
  source 1..1098: mol_type = other DNA
  misc_feature 1..1098: note = TFR1-IgG1ser-CD3.s.004.01-HA
atgatggaccaagcccgcagcgcctttagtaatctgttcggcggggaacccttatcctataccaggttcagcttggctagacaggttgatggcgacaactcacacgtcgaaatgaaactcgccgtggacgaggaggagaatgcagataataacacaaaggcgaacgtgacgaaacctaagcggtgttccggatctatttgctacggtactatagctgtaattgtgttctttcttatcggatttatgatcggctacctgggggagcccaagtctagcgacaaaacccacacaagtccaccttcccctgccccagaagctgcaggcggaccccaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaagggaggaagttacccctacgatgtgcctgactatgcctgataa

SEQ ID NO: 648 - DNA, 1032 bp, synthetic construct
  source 1..1032: mol_type = other DNA
  misc_feature 1..1032: note = HA-CD3.s.004.01-EAAAKx3-PDGFR
atggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggcggaggctgctgctaaagaggccgccgccaaggaagcagcagccaaggcaaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 649 - DNA, 978 bp, synthetic construct
  source 1..978: mol_type = other DNA
  misc_feature 1..978: note = HA-CD3.s.004.01-EAAAKx3-GPI
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggcggaggctgctgctaaagaggccgccgccaaggaagcagcagccaaggcacagcttgagaacgggggcacatctctctccgaaaagactgtcctgttactggtgacccctttcttggctgccgcatggagcctgcacccctgataa

SEQ ID NO: 650 - DNA, 1035 bp, synthetic construct
  source 1..1035: mol_type = other DNA
  misc_feature 1..1035: note = HA-CD3.s.004.01-EAAAKx3-VSVGan
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggcggaggctgctgctaaagaggccgccgccaaggaagcagcagccaaggcatcctctatcgccagcttcttcttcatcatcgggctgattatcggcctgttcctggtgctgagagtgggcatccacctgtgcatcaagctgaagcacaccaagaagcggcagatctacaccgacatcgagatgaaccggctgggcaaatgataa

SEQ ID NO: 651 - DNA, 1314 bp, synthetic construct
  source 1..1314: mol_type = other DNA
  misc_feature 1..1314: note = HA-CD3.s.004.01-EAAAKx3-CD8aTm
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggcggaggctgctgctaaagaggccgccgccaaggaagcagcagccaaggcaattgcaagtcaaccactttctttacgacccgaggcctgtaggcccgctgctgggggtgctgttcataccaggggcctggacttcgcctgcgacatatacatttgggcccctcttgcaggcacttgtggagtgctgttgttatccctggtcatcaccctatattgtaatcaccgtaatcgccggcgagtgtgtaaatgccctcggccagtggttatcgctagccagccgctcagcctgaggccagaagcgtgccgccctgcagcaggaggggccgtccatacacggggcctcgattttgcctgtgatatttatatctgggcccccctcgcggggacgtgcggtgtcctgttgctgtcactggtgatcacactctactgcaaccacagaaacaggcgcagagtgtgtaagtgccctagacccgtagtgtgataa

SEQ ID NO: 652 - DNA, 1005 bp, synthetic construct
  source 1..1005: mol_type = other DNA
  misc_feature 1..1005: note = HA-CD3.s.004.01-EAAAKx3-HLADRB
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggcggaggctgctgctaaagaggccgccgccaaggaagcagcagccaaggcaatgctgtctggtgtcggaggttttgtgctcggactcttgtttctgggggccggccttttcatctacttcaggaaccagaagggccacagcgggttacaacccaccggcttcctgtcctgataa

SEQ ID NO: 653 - DNA, 990 bp, synthetic construct
  source 1..990: mol_type = other DNA
  misc_feature 1..990: note = HA-CD3.s.004.01-EAAAKx3-CD49d
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggcggaggctgctgctaaagaggccgccgccaaggaagcagcagccaaggcaatagtgatcatcagctccagtctgctgttagggctcattgttttgcttctgatttcttatgtcatgtggaaagccggctttttcaagaggcagtacaagtcatgataa

SEQ ID NO: 654 - DNA, 1086 bp, synthetic construct
  source 1..1086: mol_type = other DNA
  misc_feature 1..1086: note = TFR1-EAAAKx3-CD3.s.004.01-HA
gccaccatgatggaccaagcccgcagcgcctttagtaatctgttcggcggggaacccttatcctataccaggttcagcttggctagacaggttgatggcgacaactcacacgtcgaaatgaaactcgccgtggacgaggaggagaatgcagataataacacaaaggcgaacgtgacgaaacctaagcggtgttccggatctatttgctacggtactatagctgtaattgtgttctttcttatcggatttatgatcggctacctgggggcggaggctgctgctaaagaggccgccgccaaggaagcagcagccaaggcacaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaagggaggaagttacccctacgatgtgcctgactatgcctgataa

SEQ ID NO: 655 - DNA, 1044 bp, synthetic construct
  source 1..1044: mol_type = other DNA
  misc_feature 1..1044: note = HA-CD3.s.001.01-G4SP-DGFR
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgggggcggtggaagcaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 656 - DNA, 1047 bp, synthetic construct
  source 1..1047: mol_type = other DNA
  misc_feature 1..1047: note = HA-CD3.s.001.01-AEAAAK-PDGFR
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgcggaggctgctgctaaaaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 657 - DNA, 1206 bp, synthetic construct
  source 1..1206: mol_type = other DNA
  misc_feature 1..1206: note = HA-CD3.s.001.01-IgG3ser-PDGFR
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgaactcaaaacacccctgggcgacacaacgcacacctcacctagaagccccgaacccaagtcatccgataccccaccgcctagtccccggtctcctgagccaaaatccagcgatactcctccaccgagtcctaggagccctgagccaaagtcttcggacactcccccaccatcccccaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 658 - DNA, 1155 bp, synthetic construct
  source 1..1155: mol_type = other DNA
  misc_feature 1..1155: note = HA-CD3.s.001.01-CD28-PDGFR
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgccgcagctattgaggtgatgtatcctccaccctacctcgacaatgaaaaaagcaacgggaccataatccacgttaagggaaaacatttgtgcccttctccactgttcccgggcccctccaagcccaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 659 - DNA, 1206 bp, synthetic construct
  source 1..1206: mol_type = other DNA
  misc_feature 1..1206: note = HA-CD3.s.001.01-IgG3hng-PDGFR
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgaactcaagacccctctgggcgacactacacacacttgtcccagatgccctgaacctaagtcttgtgatacgcccccaccctgtccccggtgcccagagcctaaatcctgtgacacacccccaccatgccctaggtgcccagagccgaaaagctgcgatacccctcccccgtgtccaaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 660 - DNA, 1098 bp, synthetic construct
  source 1..1098: mol_type = other DNA
  misc_feature 1..1098: note = HA-CD3.s.001.01-IgG1hng-PDGFR
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgaacctaaaagctgcgacaagacacacacctgccccccatgtcccgcccctgaggcagctggcgggccaaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 661 - DNA, 1065 bp, synthetic construct
  source 1..1065: mol_type = other DNA
  misc_feature 1..1065: note = HA-CD3.s.001.01-IgG4g-PDGFR
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgagtccaagtacggacctccgtgtcccagctgcccaaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 662 - DNA, 996 bp, synthetic construct
  source 1..996: mol_type = other DNA
  misc_feature 1..996: note = HA-CD3.s.001.01G4SICAM1
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgggggcggtggaagcatagtgattatcacagttgtcgccgccgctgtgattatgggaaccgcaggcctgagcacttatttgtacaaccggcagagaaagatcaagaaatacaggctctgataa

SEQ ID NO: 663 - DNA, 1026 bp, synthetic construct
  source 1..1026: mol_type = other DNA
  misc_feature 1..1026: note = HA-CD3.s.001.01-G4Sx3-ICAM1
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgggggcggcgggtccggaggtggaggttctgggggcggcggaagcatagtgattatcacagttgtcgccgccgctgtgattatgggaaccgcaggcctgagcacttatttgtacaaccggcagagaaagatcaagaaatacaggctctgataa

SEQ ID NO: 664 - DNA, 999 bp, synthetic construct
  source 1..999: mol_type = other DNA
  misc_feature 1..999: note = HA-CD3.s.001.01-AEAAAK-ICAM1
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgcggaggctgctgctaaaatagtgattatcacagttgtcgccgccgctgtgattatgggaaccgcaggcctgagcacttatttgtacaaccggcagagaaagatcaagaaatacaggctctgataa

SEQ ID NO: 665 - DNA, 1167 bp, synthetic construct
  source 1..1167: mol_type = other DNA
  misc_feature 1..1167: note = HA-CD3.s.001.01-CD8ah-ICAM1
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgcgctttccaactctattatgtacttcagtcatttcgttcccgtctttctgcctgcaaagcccacaaccacgcctgccccaaggccacccacacccgcccctactatcgcaagccagcctctcagcctgcggccggaggcttcaagaccagctgccggcggagccgtgcacacccgcgggttggacatagtgattatcacagttgtcgccgccgctgtgattatgggaaccgcaggcctgagcacttatttgtacaaccggcagagaaagatcaagaaatacaggctctgataa

SEQ ID NO: 666 - DNA, 1158 bp, synthetic construct
  source 1..1158: mol_type = other DNA
  misc_feature 1..1158: note = HA-CD3.s.001.01-IgG3ser-ICAM1
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgaactcaaaacacccctgggcgacacaacgcacacctcacctagaagccccgaacccaagtcatccgataccccaccgcctagtccccggtctcctgagccaaaatccagcgatactcctccaccgagtcctaggagccctgagccaaagtcttcggacactcccccaccatcccccatagtgattatcacagttgtcgccgccgctgtgattatgggaaccgcaggcctgagcacttatttgtacaaccggcagagaaagatcaagaaatacaggctctgataa

SEQ ID NO: 667 - DNA, 1017 bp, synthetic construct
  source 1..1017: mol_type = other DNA
  misc_feature 1..1017: note = HA-CD3.s.001.01-IgG4-ser-ICAM1
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgagtctaagtacggcccgcccagccctccctccccaatagtgattatcacagttgtcgccgccgctgtgattatgggaaccgcaggcctgagcacttatttgtacaaccggcagagaaagatcaagaaatacaggctctgataa

SEQ ID NO: 668 - DNA, 1107 bp, synthetic construct
  source 1..1107: mol_type = other DNA
  misc_feature 1..1107: note = HA-CD3.s.001.01-CD28-ICAM1
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgccgcagctattgaggtgatgtatcctccaccctacctcgacaatgaaaaaagcaacgggaccataatccacgttaagggaaaacatttgtgcccttctccactgttcccgggcccctccaagcccatagtgattatcacagttgtcgccgccgctgtgattatgggaaccgcaggcctgagcacttatttgtacaaccggcagagaaagatcaagaaatacaggctctgataa

SEQ ID NO: 669 - DNA, 1158 bp, synthetic construct
  source 1..1158: mol_type = other DNA
  misc_feature 1..1158: note = HA-CD3.s.001.01-IgG3hng-ICAM1
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgaactcaagacccctctgggcgacactacacacacttgtcccagatgccctgaacctaagtcttgtgatacgcccccaccctgtccccggtgcccagagcctaaatcctgtgacacacccccaccatgccctaggtgcccagagccgaaaagctgcgatacccctcccccgtgtccaatagtgattatcacagttgtcgccgccgctgtgattatgggaaccgcaggcctgagcacttatttgtacaaccggcagagaaagatcaagaaatacaggctctgataa

SEQ ID NO: 670 - DNA, 1050 bp, synthetic construct
  source 1..1050: mol_type = other DNA
  misc_feature 1..1050: note = HA-CD3.s.001.01-IgG1hng-ICAM1
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgaacctaaaagctgcgacaagacacacacctgccccccatgtcccgcccctgaggcagctggcgggccaatagtgattatcacagttgtcgccgccgctgtgattatgggaaccgcaggcctgagcacttatttgtacaaccggcagagaaagatcaagaaatacaggctctgataa

SEQ ID NO: 671 - DNA, 1017 bp, synthetic construct
  source 1..1017: mol_type = other DNA
  misc_feature 1..1017: note = HA-CD3.s.001.01-IgG4g-ICAM1
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgagtccaagtacggacctccgtgtcccagctgcccaatagtgattatcacagttgtcgccgccgctgtgattatgggaaccgcaggcctgagcacttatttgtacaaccggcagagaaagatcaagaaatacaggctctgataa

SEQ ID NO: 672 - DNA, 1002 bp, synthetic construct
  source 1..1002: mol_type = other DNA
  misc_feature 1..1002: note = HA-CD3.s.004.01G4SPDGFR
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggggggcggtggaagcaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 673 - DNA, 1032 bp, synthetic construct
  source 1..1032: mol_type = other DNA
  misc_feature 1..1032: note = HA-CD3.s.004.01-G4Sx3-PDGFR
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggggggcggcgggtccggaggtggaggttctgggggcggcggaagcaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 674 - DNA, 1005 bp, synthetic construct
  source 1..1005: mol_type = other DNA
  misc_feature 1..1005: note = HA-CD3.s.004.01-AEAAAK-PDGFR
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggcggaggctgctgctaaaaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 675 - DNA, 1173 bp, synthetic construct
  source 1..1173: mol_type = other DNA
  misc_feature 1..1173: note = HA-CD3.s.004.01-CD8ah-PDGFR
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggcgctttccaactctattatgtacttcagtcatttcgttcccgtctttctgcctgcaaagcccacaaccacgcctgccccaaggccacccacacccgcccctactatcgcaagccagcctctcagcctgcggccggaggcttcaagaccagctgccggcggagccgtgcacacccgcgggttggacaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 676 - DNA, 1164 bp, synthetic construct
  source 1..1164: mol_type = other DNA
  misc_feature 1..1164: note = HA-CD3.s.004.01-IgG3ser-PDGFR
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggaactcaaaacacccctgggcgacacaacgcacacctcacctagaagccccgaacccaagtcatccgataccccaccgcctagtccccggtctcctgagccaaaatccagcgatactcctccaccgagtcctaggagccctgagccaaagtcttcggacactcccccaccatcccccaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 677 - DNA, 1023 bp, synthetic construct
  source 1..1023: mol_type = other DNA
  misc_feature 1..1023: note = HA-CD3.s.004.01-IgG4-ser-PDGFR
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggagtctaagtacggcccgcccagccctccctccccaaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 678 - DNA, 1113 bp, synthetic construct
  source 1..1113: mol_type = other DNA
  misc_feature 1..1113: note = HA-CD3.s.004.01-CD28-PDGFR
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggccgcagctattgaggtgatgtatcctccaccctacctcgacaatgaaaaaagcaacgggaccataatccacgttaagggaaaacatttgtgcccttctccactgttcccgggcccctccaagcccaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 679 - DNA, 1164 bp, synthetic construct
  source 1..1164: mol_type = other DNA
  misc_feature 1..1164: note = HA-CD3.s.004.01-IgG3hng-PDGFR
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggaactcaagacccctctgggcgacactacacacacttgtcccagatgccctgaacctaagtcttgtgatacgcccccaccctgtccccggtgcccagagcctaaatcctgtgacacacccccaccatgccctaggtgcccagagccgaaaagctgcgatacccctcccccgtgtccaaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 680 - DNA, 1056 bp, synthetic construct
  source 1..1056: mol_type = other DNA
  misc_feature 1..1056: note = HA-CD3.s.004.01-IgG1hng-PDGFR
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggaacctaaaagctgcgacaagacacacacctgccccccatgtcccgcccctgaggcagctggcgggccaaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 681 - DNA, 1023 bp, synthetic construct
  source 1..1023: mol_type = other DNA
  misc_feature 1..1023: note = HA-CD3.s.004.01-IgG4g-PDGFR
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggagtccaagtacggacctccgtgtcccagctgcccaaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 682 - DNA, 954 bp, synthetic construct
  source 1..954: mol_type = other DNA
  misc_feature 1..954: note = HA-CD3.s.004.01G4SICAM1
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggggggcggtggaagcatagtgattatcacagttgtcgccgccgctgtgattatgggaaccgcaggcctgagcacttatttgtacaaccggcagagaaagatcaagaaatacaggctctgataa

SEQ ID NO: 683 - DNA, 984 bp, synthetic construct
  source 1..984: mol_type = other DNA
  misc_feature 1..984: note = HA-CD3.s.004.01-G4Sx3-ICAM1
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggggggcggcgggtccggaggtggaggttctgggggcggcggaagcatagtgattatcacagttgtcgccgccgctgtgattatgggaaccgcaggcctgagcacttatttgtacaaccggcagagaaagatcaagaaatacaggctctgataa

SEQ ID NO: 684 - DNA, 957 bp, synthetic construct
  source 1..957: mol_type = other DNA
  misc_feature 1..957: note = HA-CD3.s.004.01-AEAAAK-ICAM1
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggcggaggctgctgctaaaatagtgattatcacagttgtcgccgccgctgtgattatgggaaccgcaggcctgagcacttatttgtacaaccggcagagaaagatcaagaaatacaggctctgataa

SEQ ID NO: 685 - DNA, 1125 bp, synthetic construct
  source 1..1125: mol_type = other DNA
  misc_feature 1..1125: note = HA-CD3.s.004.01-CD8ah-ICAM1
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggcgctttccaactctattatgtacttcagtcatttcgttcccgtctttctgcctgcaaagcccacaaccacgcctgccccaaggccacccacacccgcccctactatcgcaagccagcctctcagcctgcggccggaggcttcaagaccagctgccggcggagccgtgcacacccgcgggttggacatagtgattatcacagttgtcgccgccgctgtgattatgggaaccgcaggcctgagcacttatttgtacaaccggcagagaaagatcaagaaatacaggctctgataa

SEQ ID NO: 686 - DNA, 1116 bp, synthetic construct
  source 1..1116: mol_type = other DNA
  misc_feature 1..1116: note = HA-CD3.s.004.01-IgG3ser-ICAM1
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggaactcaaaacacccctgggcgacacaacgcacacctcacctagaagccccgaacccaagtcatccgataccccaccgcctagtccccggtctcctgagccaaaatccagcgatactcctccaccgagtcctaggagccctgagccaaagtcttcggacactcccccaccatcccccatagtgattatcacagttgtcgccgccgctgtgattatgggaaccgcaggcctgagcacttatttgtacaaccggcagagaaagatcaagaaatacaggctctgataa

SEQ ID NO: 687 - DNA, 1065 bp, synthetic construct
  source 1..1065: mol_type = other DNA
  misc_feature 1..1065: note = HA-CD3.s.004.01-CD28-ICAM1
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggccgcagctattgaggtgatgtatcctccaccctacctcgacaatgaaaaaagcaacgggaccataatccacgttaagggaaaacatttgtgcccttctccactgttcccgggcccctccaagcccatagtgattatcacagttgtcgccgccgctgtgattatgggaaccgcaggcctgagcacttatttgtacaaccggcagagaaagatcaagaaatacaggctctgataa

SEQ ID NO: 688 - DNA, 1116 bp, synthetic construct
  source 1..1116: mol_type = other DNA
  misc_feature 1..1116: note = HA-CD3.s.004.01-IgG3hng-ICAM1
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggaactcaagacccctctgggcgacactacacacacttgtcccagatgccctgaacctaagtcttgtgatacgcccccaccctgtccccggtgcccagagcctaaatcctgtgacacacccccaccatgccctaggtgcccagagccgaaaagctgcgatacccctcccccgtgtccaatagtgattatcacagttgtcgccgccgctgtgattatgggaaccgcaggcctgagcacttatttgtacaaccggcagagaaagatcaagaaatacaggctctgataa

SEQ ID NO: 689 - DNA, 1008 bp, synthetic construct
  source 1..1008: mol_type = other DNA
  misc_feature 1..1008: note = HA-CD3.s.004.01-IgG1hng-ICAM1
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggaacctaaaagctgcgacaagacacacacctgccccccatgtcccgcccctgaggcagctggcgggccaatagtgattatcacagttgtcgccgccgctgtgattatgggaaccgcaggcctgagcacttatttgtacaaccggcagagaaagatcaagaaatacaggctctgataa

SEQ ID NO: 690 - DNA, 975 bp, synthetic construct
  source 1..975: mol_type = other DNA
  misc_feature 1..975: note = HA-CD3.s.004.01-IgG4g-ICAM1
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggagtccaagtacggacctccgtgtcccagctgcccaatagtgattatcacagttgtcgccgccgctgtgattatgggaaccgcaggcctgagcacttatttgtacaaccggcagagaaagatcaagaaatacaggctctgataa

SEQ ID NO: 691 - DNA, 1098 bp, synthetic construct
  source 1..1098: mol_type = other DNA
  misc_feature 1..1098: note = RevisedHA-UMTxOKT3
ggcaccatggaaacagacaccctcctgctgtgggtcctactgttatgggttcctggtagcacaggctacccctacgatgtgcctgactatgccggaggaagtcaagtgcagctgcaacagagcggcgctgagctggcgagaccgggcgcctctgttaagatgagttgcaaagcgtctggctatacgtttacgcgctacaccatgcactgggtgaaacagcggcctggccagggcctagagtggattggatacatcaacccctcaagagggtacactaactacaatcagaagttcaaggataaagctaccctcacaactgacaagtcatcttctacagcctatatgcaactgtcctccctcacctccgaggactcagcggtgtactactgtgcaagatattatgacgatcattattgtctggattactggggccaggggaccacccttacggttagctccagcggcgggggaggaagcggaggcgggggtagcgggggaggtggttctcagattgtgctcacacaaagcccagccatcatgtctgcttccccgggggaaaaagtcaccatgacctgtagcgcctcctcaagtgtcagttatatgaattggtatcagcagaagtcgggaacaagccccaagagatggatttacgatacctccaaactggcaagcggggtgcccgcccacttcaggggttcaggctccggcactagttactcacttaccatctccggaatggaggccgaagatgcagcaacttactattgccagcagtggagtagtaaccctttcacgtttgggtctggcacaaaattggaaatcaatcgttcagatccaaccactacacccgctcctaggcccccaaccccggctccaactatagcctcgcagccactttccctccggcccgaggcctgtcgccccgctgctgggggagccgtccacacacgcggactggactttgcctgcgacatctacatttgggcaccattggcaggcacttgcggtgtactgttgttatctcttgtaataactctgtattgcaaccatcgaaatcggaggagggtgtgtaagtgccctcgacctgttgtgtgataa

SEQ ID NO: 692 - DNA, 1062 bp, synthetic construct
  source 1..1062: mol_type = other DNA
  misc_feature 1..1062: note = RevisedOKT3
ggcaccatggaaacagacaccctcctgctgtgggtcctactgttatgggttcctggtagcacaggccaagtgcagctgcaacagagcggcgctgagctggcgagaccgggcgcctctgttaagatgagttgcaaagcgtctggctatacgtttacgcgctacaccatgcactgggtgaaacagcggcctggccagggcctagagtggattggatacatcaacccctcaagagggtacactaactacaatcagaagttcaaggataaagctaccctcacaactgacaagtcatcttctacagcctatatgcaactgtcctccctcacctccgaggactcagcggtgtactactgtgcaagatattatgacgatcattattgtctggattactggggccaggggaccacccttacggttagctccagcggcgggggaggaagcggaggcgggggtagcgggggaggtggttctcagattgtgctcacacaaagcccagccatcatgtctgcttccccgggggaaaaagtcaccatgacctgtagcgcctcctcaagtgtcagttatatgaattggtatcagcagaagtcgggaacaagccccaagagatggatttacgatacctccaaactggcaagcggggtgcccgcccacttcaggggttcaggctccggcactagttactcacttaccatctccggaatggaggccgaagatgcagcaacttactattgccagcagtggagtagtaaccctttcacgtttgggtctggcacaaaattggaaatcaatcgttcagatccaaccactacacccgctcctaggcccccaaccccggctccaactatagcctcgcagccactttccctccggcccgaggcctgtcgccccgctgctgggggagccgtccacacacgcggactggactttgcctgcgacatctacatttgggcaccattggcaggcacttgcggtgtactgttgttatctcttgtaataactctgtattgcaaccatcgaaatcggaggagggtgtgtaagtgccctcgacctgttgtgtgataa

SEQ ID NO: 693 - DNA, 1080 bp, synthetic construct
  source 1..1080: mol_type = other DNA
  misc_feature 1..1080: note = HA-ASGR1.s.017.02-IgG1ser-PDGF
gccaccatggagactgacacactgctcttatgggtgcttctgttgtgggtccccggcagcaccggggattacccctacgatgtgcctgactatgccggaggaagtgaggttcagctcgtagagtccgggggcggcctcgtgcaacctgggggctctcttcgtctctcttgtgccgcttcaggctttaccttctccagttataacatgaactgggtgcgccaggcccctggcaaagggctggaatgggtgtcttacattagccgctctagcaataccaagcactatgcagactcggtgaagggtaggtttaccataagcagggacaacgccaaaaattcattgtacttacaaatgaacagtttacgagacgaagacactgctgtgtactactgcgctcgggacaggagcggatcttacggatatttctactattacggcctggatgtctgggggcagggaaccactgtcacagtcagttccggaggtggaggcagcggaggcggggggagtggaggggggggttcacagagtgtcctgacccagcctccatctgcatcaggcacgcccggccaacgggttacgatttcatgttccgggagcagtagcaatatcgggtccaatgccgtgaactggtatcagcagctgcccggaacagcgccaaaactgctgatctattcaaataatcagagaccctctggcgtgccggacagattcagcggatctaagagcggcacatccgcatccctcgccatctccggactgcagtcggaagatgaggctgattactattgcgcggcctgggatgattcccttaacggtgtggtttttggcggtggtacaaagttgactgtactagagcccaagtctagcgacaaaacccacacaagtccaccttcccctgccccagaagctgcaggcggacccaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 694 - DNA, 806 bp, synthetic construct
  source 1..806: mol_type = other DNA
  misc_feature 1..806: note = pCAGGS-SS-GGlandingpad-RFP
gccaccatggagactgacacactgctcttatgggtgcttctgttgtgggtccccggcagcaccggggatggagacggaatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgatatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacttacgtcgagcagcacgaggtggctgtggccagatactgcgacctccctagcaaactggggcacaaacttaattgacgtctctctattga

SEQ ID NO: 695 - DNA, 1062 bp, synthetic construct
  source 1..1062: mol_type = other DNA
  misc_feature 1..1062: note = HA-CD3.s.001.01-IgG4PDGFR-Dob
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgagtccaagtacggacctccgtgtcccagctgcccatctgcctgcagagatctgaagagatgtccccgcgaactaccgcccaccctgcttggcaattcttccgaagaggaaagtcagctcgaaactaacgtgacatactgggaggaggagcaggagtttgaggttgtatcgacgctgcgattgtgataa

SEQ ID NO: 696 - DNA, 915 bp, synthetic construct
  source 1..915: mol_type = other DNA
  misc_feature 1..915: note = HA-CD3.s.001.01-IgG4PDGFR-Dob2
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgagtccaagtacggacctccgtgtcccagctgcccatgataa

SEQ ID NO: 697 - DNA, 1032 bp, synthetic construct
  source 1..1032: mol_type = other DNA
  misc_feature 1..1032: note = HA-CD3.s.001.01-EAAAKx3-ICAM1
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgcggaggctgctgctaaagaggccgccgccaaggaagcagcagccaaggcaatagtgattatcacagttgtcgccgccgctgtgattatgggaaccgcaggcctgagcacttatttgtacaaccggcagagaaagatcaagaaatacaggctctgataa

SEQ ID NO: 698 - DNA, 1332 bp, synthetic construct
  source 1..1332: mol_type = other DNA
  misc_feature 1..1332: note = HA-CD3.s.004.01-IgG1ser-CD8aTm
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggagcccaagtctagcgacaaaacccacacaagtccaccttcccctgccccagaagctgcaggcggacccattgcaagtcaaccactttctttacgacccgaggcctgtaggcccgctgctgggggtgctgttcataccaggggcctggacttcgcctgcgacatatacatttgggcccctcttgcaggcacttgtggagtgctgttgttatccctggtcatcaccctatattgtaatcaccgtaatcgccggcgagtgtgtaaatgccctcggccagtggttatcgctagccagccgctcagcctgaggccagaagcgtgccgccctgcagcaggaggggccgtccatacacggggcctcgattttgcctgtgatatttatatctgggcccccctcgcggggacgtgcggtgtcctgttgctgtcactggtgatcacactctactgcaaccacagaaacaggcgcagagtgtgtaagtgccctagacccgtagtgtgataa

SEQ ID NO: 699 - DNA, 990 bp, synthetic construct
  source 1..990: mol_type = other DNA
  misc_feature 1..990: note = HA-CD3.s.004.01-EAAAKx3-ICAM1
gccaccatggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggcggaggctgctgctaaagaggccgccgccaaggaagcagcagccaaggcaatagtgattatcacagttgtcgccgccgctgtgattatgggaaccgcaggcctgagcacttatttgtacaaccggcagagaaagatcaagaaatacaggctctgataa

SEQ ID NO: 700 - DNA, 1074 bp, synthetic construct
  source 1..1074: mol_type = other DNA
  misc_feature 1..1074: note = HA-CD3.s.001.01-G4Sx3-PDGFR
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgggggcggcgggtccggaggtggaggttctgggggcggcggaagcaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 701 - DNA, 1215 bp, synthetic construct
  source 1..1215: mol_type = other DNA
  misc_feature 1..1215: note = HA-CD3.s.001.01-CD8ah-PDGFR
gccaccatggagactgacacactgctcttatgggtgcttctgttgtgggtccccggcagcaccggggattacccctacgatgtgcctgactatgccggaggaagtgcggctcagccggcatcagacataaaactccaacagtcaggcgctgaattggctaggccaggggcaagcgttaagatgtcttgtaaaacttccggctacacttttacgcggtacaccatgcactgggtcaaacagcgacccggccaggggcttgagtggatcggctacattaacccctcgagaggatacaccaactacaatcagaagtttaaggacaaagccactctaacaaccgacaagtcttcctcaaccgcatatatgcagttgtccagcctgacatctgaggattccgccgtgtattactgcgcacgttattatgacgatcattattgtcttgactactggggacagggcactacactgacagtgtcctctgttgagggtggaggcggctctggggggggagggagcggagggggtgggagcgtggacgatatccagctcacccagagtcctgccattatgtctgccagtcctggagaaaaggtgaccatgacgtgcagggctagctcctcagtcagctacatgaactggtatcaacagaagtcaggcactagtcccaagcgctggatctacgatacctccaaggtggccagcggagtaccatatcggttcagtggcagtggtagcggtacatcctattcgttaactatttcaagtatggaagctgaggatgccgcaacatactactgccaacaatggtctagcaatcctctgaccttcggcgccggtacgaaactggagctgaaagcggccgctagagggtccgcgctttccaactctattatgtacttcagtcatttcgttcccgtctttctgcctgcaaagcccacaaccacgcctgccccaaggccacccacacccgcccctactatcgcaagccagcctctcagcctgcggccggaggcttcaagaccagctgccggcggagccgtgcacacccgcgggttggacaacgctgtcggccaggacacccaagaggtgatagtggtgccacacagcctgcccttcaaggtagtggtcatctccgcaatccttgccctcgttgttttgacaattatttctctgattatcttaatcatgctgtggcagaaaaagcctaggtgataa

SEQ ID NO: 702 - DNA, 969 bp, synthetic construct
  source 1..969: mol_type = other DNA
  misc_feature 1..969: note = HA-+C634:C710CD3.s.004.01-IgG4-ser-ICAM1
atggagactgacacactgctcttatgggtgcttctgttgtgggtccccggcagcaccggggattacccctacgatgtgcctgactatgccggaggaagtcaggtgcagctgcagcagagcggagccgagctggcacgtccaggcgccagtgtgaagatgtcctgtaaagcgtcagggtacaccttcaccagatacaccatgcattgggttaaacagcgccccggccaggggcttgaatggattggctacataaacccttcaaggggttatactaactataaccaaaagttcaaagacaaagctacactgacaactgacaagtcaagtagcaccgcttacatgcagctttcatctctgacctcagaggatagtgcagtctattattgcgcgcgatattacgacgatcactacagcctcgattactggggacaaggaacgacattgactgtatcgtccgggggcggtgggtctggcggaggcgggagtggaggaggcggtagcgacattgttttaacgcaaagccctgctatcatcgtgatttccgcctccccaggcgagaaagtcactatgacctgctcggccagttcctctgtgtcttacatgaattggtatcagcagaagtccggcacctctccgaaaaggtggatctatgatacttccaagctggcctctggcgtgcccgcacactttcggggatcagggtccgggacaagctacagcctaacaataagcggtatggaagctgaggacgcagccacctactattgtcagcaatggagcagtaatcccttcacttttgggtctggtacaaagctcgaaatcaaggagtctaagtacggcccgcccagccctccctccccaatagtgattatcacagttgtcgccgccgctgtgattatgggaaccgcaggcctgagcacttatttgtacaaccggcagagaaagatcaagaaatacaggctctgataa

SEQ ID NO: 703 - protein, 362 aa, synthetic construct
  REGION 1..362: note = HA-CD3.s.001.01-IgG1ser-PDGFR
METDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSEPKSSDKTHTSPPSPAPEAAGGPNAVGQDTQEVIVVPHSLPFKVVVISAILALVVLTIISLIILIMLWQKKPR

SEQ ID NO: 704 - protein, 342 aa, synthetic construct
  REGION 1..342: note = HA-CD3.s.001.01-IgG1ser-GPI
METDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSEPKSSDKTHTSPPSPAPEAAGGPQLENGGTSLSEKTVLLLVTPFLAAAWSLHP

SEQ ID NO: 705 - protein, 361 aa, synthetic construct
  REGION 1..361: note = HA-CD3.s.001.01-IgG1ser-VSVGan
METDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSEPKSSDKTHTSPPSPAPEAAGGPSSIASFFFIIGLIIGLFLVLRVGIHLCIKLKHTKKRQIYTDIEMNRLGK

SEQ ID NO: 706 - protein, 454 aa, synthetic construct
  REGION 1..454: note = HA-CD3.s.001.01-IgG1ser-CD8aTm
METDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSEPKSSDKTHTSPPSPAPEAAGGPIASQPLSLRPEACRPAAGGAVHTRGLDFACDIYIWAPLAGTCGVLLLSLVITLYCNHRNRRRVCKCPRPVVIASQPLSLRPEACRPAAGGAVHTRGLDFACDIYIWAPLAGTCGVLLLSLVITLYCNHRNRRRVCKCPRPVV

SEQ ID NO: 707 - protein, 346 aa, synthetic construct
  REGION 1..346: note = HA-CD3.s.001.01-IgG1ser-ICAM1
METDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSEPKSSDKTHTSPPSPAPEAAGGPIVIITVVAAAVIMGTAGLSTYLYNRQRKIKKYRL

SEQ ID NO: 708 - protein, 351 aa, synthetic construct
  REGION 1..351: note = HA-CD3.s.001.01-IgG1ser-HLADRB
METDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSEPKSSDKTHTSPPSPAPEAAGGPMLSGVGGFVLGLLFLGAGLFIYFRNQKGHSGLQPTGFLS

SEQ ID NO: 709 - protein, 346 aa, synthetic construct
  REGION 1..346: note = HA-CD3.s.001.01-IgG1ser-CD49d
METDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSEPKSSDKTHTSPPSPAPEAAGGPIVIISSSLLLGLIVLLLISYVMWKAGFFKRQYKS

SEQ ID NO: 710 - protein, 378 aa, synthetic construct
  REGION 1..378: note = TFR1-IgG1ser-CD3.s.001.01-HA
MMDQARSAFSNLFGGEPLSYTRFSLARQVDGDNSHVEMKLAVDEEENADNNTKANVTKPKRCSGSICYGTIAVIVFFLIGFMIGYLGEPKSSDKTHTSPPSPAPEAAGGPAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSGGSYPYDVPDYA

SEQ ID NO: 711 - protein, 356 aa, synthetic construct
  REGION 1..356: note = HA-CD3.s.001.01-EAAAKx3-PDGFR
METDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSAEAAAKEAAAKEAAAKANAVGQDTQEVIVVPHSLPFKVVVISAILALVVLTIISLIILIMLWQKKPR

SEQ ID NO: 712 - protein, 336 aa, synthetic construct
  REGION 1..336: note = HA-CD3.s.001.01-EAAAKx3-GPI
METDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSAEAAAKEAAAKEAAAKAQLENGGTSLSEKTVLLLVTPFLAAAWSLHP

SEQ ID NO: 713 - protein, 355 aa, synthetic construct
  REGION 1..355: note = HA-CD3.s.001.01-EAAAKx3-VSVGan
METDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSAEAAAKEAAAKEAAAKASSIASFFFIIGLIIGLFLVLRVGIHLCIKLKHTKKRQIYTDIEMNRLGK

SEQ ID NO: 714 - protein, 448 aa, synthetic construct
  REGION 1..448: note = HA-CD3.s.001.01-EAAAKx3-CD8aTm
METDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSAEAAAKEAAAKEAAAKAIASQPLSLRPEACRPAAGGAVHTRGLDFACDIYIWAPLAGTCGVLLLSLVITLYCNHRNRRRVCKCPRPVVIASQPLSLRPEACRPAAGGAVHTRGLDFACDIYIWAPLAGTCGVLLLSLVITLYCNHRNRRRVCKCPRPVV

SEQ ID NO: 715 - protein, 345 aa, synthetic construct
  REGION 1..345: note = HA-CD3.s.001.01-EAAAKx3-HLADRB
METDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSAEAAAKEAAAKEAAAKAMLSGVGGFVLGLLFLGAGLFIYFRNQKGHSGLQPTGFLS

SEQ ID NO: 716 - protein, 340 aa, synthetic construct
  REGION 1..340: note = HA-CD3.s.001.01-EAAAKx3-CD49d
METDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSAEAAAKEAAAKEAAAKAIVIISSSLLLGLIVLLLISYVMWKAGFFKRQYKS

SEQ ID NO: 717 - protein, 372 aa, synthetic construct
  REGION 1..372: note = TFR1-EAAAKx3-CD3.s.001.01-HA
MMDQARSAFSNLFGGEPLSYTRFSLARQVDGDNSHVEMKLAVDEEENADNNTKANVTKPKRCSGSICYGTIAVIVFFLIGFMIGYLGAEAAAKEAAAKEAAAKAAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSGGSYPYDVPDYA

SEQ ID NO: 718 - protein, 348 aa, synthetic construct
  REGION 1..348: note = HA-CD3.s.004.01-IgG1ser-PDGFR
METDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKEPKSSDKTHTSPPSPAPEAAGGPNAVGQDTQEVIVVPHSLPFKVVVISAILALVVLTIISLIILIMLWQKKPR

SEQ ID NO: 719 - protein, 328 aa, synthetic construct
  REGION 1..328: note = HA-CD3.s.004.01-IgG1ser-GPI
METDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKEPKSSDKTHTSPPSPAPEAAGGPQLENGGTSLSEKTVLLLVTPFLAAAWSLHP

SEQ ID NO: 720 - protein, 347 aa, synthetic construct
  REGION 1..347: note = HA-CD3.s.004.01-IgG1ser-VSVGan
METDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKEPKSSDKTHTSPPSPAPEAAGGPSSIASFFFIIGLIIGLFLVLRVGIHLCIKLKHTKKRQIYTDIEMNRLGK

SEQ ID NO: 721 - protein, 332 aa, synthetic construct
  REGION 1..332: note = HA-CD3.s.004.01-IgG1ser-ICAM1
METDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKEPKSSDKTHTSPPSPAPEAAGGPIVIITVVAAAVIMGTAGLSTYLYNRQRKIKKYRL

SEQ ID NO: 722 - protein, 337 aa, synthetic construct
  REGION 1..337: note = HA-CD3.s.004.01-IgG1ser-HLADRB
METDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKEPKSSDKTHTSPPSPAPEAAGGPMLSGVGGFVLGLLFLGAGLFIYFRNQKGHSGLQPTGFLS

SEQ ID NO: 723 - protein, 332 aa, synthetic construct
  REGION 1..332: note = HA-CD3.s.004.01-IgG1ser-CD49d
METDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKEPKSSDKTHTSPPSPAPEAAGGPIVIISSSLLLGLIVLLLISYVMWKAGFFKRQYKS

SEQ ID NO: 724 - protein, 364 aa, synthetic construct
  REGION 1..364: note = TFR1-IgG1ser-CD3.s.004.01-HA
MMDQARSAFSNLFGGEPLSYTRFSLARQVDGDNSHVEMKLAVDEEENADNNTKANVTKPKRCSGSICYGTIAVIVFFLIGFMIGYLGEPKSSDKTHTSPPSPAPEAAGGPQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKGGSYPYDVPDYA

SEQ ID NO: 725 - protein, 342 aa, synthetic construct
  REGION 1..342: note = HA-CD3.s.004.01-EAAAKx3-PDGFR
METDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKAEAAAKEAAAKEAAAKANAVGQDTQEVIVVPHSLPFKVVVISAILALVVLTIISLIILIMLWQKKPR

SEQ ID NO: 726 - protein, 324 aa, synthetic construct
  REGION 1..324: note = HA-CD3.s.004.01-EAAAKx3-GPI
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKAEAAAKEAAAKEAAAKAQLENGGTSLSEKTVLLLVTPFLAAAWSLHP

SEQ ID NO: 727 - protein, 343 aa, synthetic construct
  REGION 1..343: note = HA-CD3.s.004.01-EAAAKx3-VSVGan
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKAEAAAKEAAAKEAAAKASSIASFFFIIGLIIGLFLVLRVGIHLCIKLKHTKKRQIYTDIEMNRLGK

SEQ ID NO: 728 - protein, 436 aa, synthetic construct
  REGION 1..436: note = HA-CD3.s.004.01-EAAAKx3-CD8aTm
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKAEAAAKEAAAKEAAAKAIASQPLSLRPEACRPAAGGAVHTRGLDFACDIYIWAPLAGTCGVLLLSLVITLYCNHRNRRRVCKCPRPVVIASQPLSLRPEACRPAAGGAVHTRGLDFACDIYIWAPLAGTCGVLLLSLVITLYCNHRNRRRVCKCPRPVV

SEQ ID NO: 729 - protein, 333 aa, synthetic construct
  REGION 1..333: note = HA-CD3.s.004.01-EAAAKx3-HLADRB
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKAEAAAKEAAAKEAAAKAMLSGVGGFVLGLLFLGAGLFIYFRNQKGHSGLQPTGFLS

SEQ ID NO: 730 - protein, 328 aa, synthetic construct
  REGION 1..328: note = HA-CD3.s.004.01-EAAAKx3-CD49d
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKAEAAAKEAAAKEAAAKAIVIISSSLLLGLIVLLLISYVMWKAGFFKRQYKS

SEQ ID NO: 731 - protein, 360 aa, synthetic construct
  REGION 1..360: note = TFR1-EAAAKx3-CD3.s.004.01-HA
ATMMDQARSAFSNLFGGEPLSYTRFSLARQVDGDNSHVEMKLAVDEEENADNNTKANVTKPKRCSGSICYGTIAVIVFFLIGFMIGYLGAEAAAKEAAAKEAAAKAQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKGGSYPYDVPDYA

SEQ ID NO: 732 - protein, 346 aa, synthetic construct
  REGION 1..346: note = HA-CD3.s.001.01-G4SP-DGFR
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSGGGGSNAVGQDTQEVIVVPHSLPFKVVVISAILALVVLTIISLIILIMLWQKKPR

SEQ ID NO: 733 - protein, 347 aa, synthetic construct
  REGION 1..347: note = HA-CD3.s.001.01-AEAAAK-PDGFR
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSAEAAAKNAVGQDTQEVIVVPHSLPFKVVVISAILALVVLTIISLIILIMLWQKKPR

SEQ ID NO: 734 - protein, 400 aa, synthetic construct
  REGION 1..400: note = HA-CD3.s.001.01-IgG3ser-PDGFR
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSELKTPLGDTTHTSPRSPEPKSSDTPPPSPRSPEPKSSDTPPPSPRSPEPKSSDTPPPSPNAVGQDTQEVIVVPHSLPFKVVVISAILALVVLTIISLIILIMLWQKKPR

SEQ ID NO: 735 - protein, 383 aa, synthetic construct
  REGION 1..383: note = HA-CD3.s.001.01-CD28-PDGFR
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSAAAIEVMYPPPYLDNEKSNGTIIHVKGKHLCPSPLFPGPSKPNAVGQDTQEVIVVPHSLPFKVVVISAILALVVLTIISLIILIMLWQKKPR

SEQ ID NO: 736 - protein, 400 aa, synthetic construct
  REGION 1..400: note = HA-CD3.s.001.01-IgG3hng-PDGFR
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSELKTPLGDTTHTCPRCPEPKSCDTPPPCPRCPEPKSCDTPPPCPRCPEPKSCDTPPPCPNAVGQDTQEVIVVPHSLPFKVVVISAILALVVLTIISLIILIMLWQKKPR

SEQ ID NO: 737 - protein, 364 aa, synthetic construct
  REGION 1..364: note = HA-CD3.s.001.01-IgG1hng-PDGFR
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSEPKSCDKTHTCPPCPAPEAAGGPNAVGQDTQEVIVVPHSLPFKVVVISAILALVVLTIISLIILIMLWQKKPR

SEQ ID NO: 738 - protein, 353 aa, synthetic construct
  REGION 1..353: note = HA-CD3.s.001.01-IgG4g-PDGFR
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSESKYGPPCPSCPNAVGQDTQEVIVVPHSLPFKVVVISAILALVVLTIISLIILIMLWQKKPR

SEQ ID NO: 739 - protein, 330 aa, synthetic construct
  REGION 1..330: note = HA-CD3.s.001.01G4SICAM1
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSGGGGSIVIITVVAAAVIMGTAGLSTYLYNRQRKIKKYRL

SEQ ID NO: 740 - protein, 340 aa, synthetic construct
  REGION 1..340: note = HA-CD3.s.001.01-G4Sx3-ICAM1
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSGGGGSGGGGSGGGGSIVIITVVAAAVIMGTAGLSTYLYNRQRKIKKYRL

SEQ ID NO: 741 - protein, 331 aa, synthetic construct
  REGION 1..331: note = HA-CD3.s.001.01-AEAAAK-ICAM1
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSAEAAAKIVIITVVAAAVIMGTAGLSTYLYNRQRKIKKYRL

SEQ ID NO: 742 - protein, 387 aa, synthetic construct
  REGION 1..387: note = HA-CD3.s.001.01-CD8ah-ICAM1
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSALSNSIMYFSHFVPVFLPAKPTTTPAPRPPTPAPTIASQPLSLRPEASRPAAGGAVHTRGLDIVIITVVAAAVIMGTAGLSTYLYNRQRKIKKYRL

SEQ ID NO: 743 - protein, 384 aa, synthetic construct
  REGION 1..384: note = HA-CD3.s.001.01-IgG3ser-ICAM1
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSELKTPLGDTTHTSPRSPEPKSSDTPPPSPRSPEPKSSDTPPPSPRSPEPKSSDTPPPSPIVIITVVAAAVIMGTAGLSTYLYNRQRKIKKYRL

SEQ ID NO: 744 - protein, 337 aa, synthetic construct
  REGION 1..337: note = HA-CD3.s.001.01-IgG4-ser-ICAM1
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSESKYGPPSPPSPIVIITVVAAAVIMGTAGLSTYLYNRQRKIKKYRL

SEQ ID NO: 745 - protein, 367 aa, synthetic construct
  REGION 1..367: note = HA-CD3.s.001.01-CD28-ICAM1
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSAAAIEVMYPPPYLDNEKSNGTIIHVKGKHLCPSPLFPGPSKPIVIITVVAAAVIMGTAGLSTYLYNRQRKIKKYRL

SEQ ID NO: 746 - protein, 384 aa, synthetic construct
  REGION 1..384: note = HA-CD3.s.001.01-IgG3hng-ICAM1
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSELKTPLGDTTHTCPRCPEPKSCDTPPPCPRCPEPKSCDTPPPCPRCPEPKSCDTPPPCPIVIITVVAAAVIMGTAGLSTYLYNRQRKIKKYRL

SEQ ID NO: 747 - protein, 348 aa, synthetic construct
  REGION 1..348: note = HA-CD3.s.001.01-IgG1hng-ICAM1
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSEPKSCDKTHTCPPCPAPEAAGGPIVIITVVAAAVIMGTAGLSTYLYNRQRKIKKYRL

SEQ ID NO: 748 - protein, 337 aa, synthetic construct
  REGION 1..337: note = HA-CD3.s.001.01-IgG4g-ICAM1
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSESKYGPPCPSCPIVIITVVAAAVIMGTAGLSTYLYNRQRKIKKYRL

SEQ ID NO: 749 - protein, 332 aa, synthetic construct
  REGION 1..332: note = HA-CD3.s.004.01G4SPDGFR
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKGGGGSNAVGQDTQEVIVVPHSLPFKVVVISAILALVVLTIISLIILIMLWQKKPR

SEQ ID NO: 750 - protein, 342 aa, synthetic construct
  REGION 1..342: note = HA-CD3.s.004.01-G4Sx3-PDGFR
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKGGGGSGGGGSGGGGSNAVGQDTQEVIVVPHSLPFKVVVISAILALVVLTIISLIILIMLWQKKPR

SEQ ID NO: 751 - protein, 333 aa, synthetic construct
  REGION 1..333: note = HA-CD3.s.004.01-AEAAAK-PDGFR
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKAEAAAKNAVGQDTQEVIVVPHSLPFKVVVISAILALVVLTIISLIILIMLWQKKPR

SEQ ID NO: 752 - protein, 389 aa, synthetic construct
  REGION 1..389: note = HA-CD3.s.004.01-CD8ah-PDGFR
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKALSNSIMYFSHFVPVFLPAKPTTTPAPRPPTPAPTIASQPLSLRPEASRPAAGGAVHTRGLDNAVGQDTQEVIVVPHSLPFKVVVISAILALVVLTIISLIILIMLWQKKPR

SEQ ID NO: 753 - protein, 386 aa, synthetic construct
  REGION 1..386: note = HA-CD3.s.004.01-IgG3ser-PDGFR
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKELKTPLGDTTHTSPRSPEPKSSDTPPPSPRSPEPKSSDTPPPSPRSPEPKSSDTPPPSPNAVGQDTQEVIVVPHSLPFKVVVISAILALVVLTIISLIILIMLWQKKPR

SEQ ID NO: 754 - protein, 339 aa, synthetic construct
  REGION 1..339: note = HA-CD3.s.004.01-IgG4-ser-PDGFR
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKESKYGPPSPPSPNAVGQDTQEVIVVPHSLPFKVVVISAILALVVLTIISLIILIMLWQKKPR

SEQ ID NO: 755 - protein, 369 aa, synthetic construct
  REGION 1..369: note = HA-CD3.s.004.01-CD28-PDGFR
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKAAAIEVMYPPPYLDNEKSNGTIIHVKGKHLCPSPLFPGPSKPNAVGQDTQEVIVVPHSLPFKVVVISAILALVVLTIISLIILIMLWQKKPR

SEQ ID NO: 756 - protein, 386 aa, synthetic construct
  REGION 1..386: note = HA-CD3.s.004.01-IgG3hng-PDGFR
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKELKTPLGDTTHTCPRCPEPKSCDTPPPCPRCPEPKSCDTPPPCPRCPEPKSCDTPPPCPNAVGQDTQEVIVVPHSLPFKVVVISAILALVVLTIISLIILIMLWQKKPR

SEQ ID NO: 757 - protein, 350 aa, synthetic construct
  REGION 1..350: note = HA-CD3.s.004.01-IgG1hng-PDGFR
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKEPKSCDKTHTCPPCPAPEAAGGPNAVGQDTQEVIVVPHSLPFKVVVISAILALVVLTIISLIILIMLWQKKPR

SEQ ID NO: 758 - protein, 339 aa, synthetic construct
  REGION 1..339: note = HA-CD3.s.004.01-IgG4g-PDGFR
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKESKYGPPCPSCPNAVGQDTQEVIVVPHSLPFKVVVISAILALVVLTIISLIILIMLWQKKPR

SEQ ID NO: 759 - protein, 316 aa, synthetic construct
  REGION 1..316: note = HA-CD3.s.004.01G4SICAM1
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKGGGGSIVIITVVAAAVIMGTAGLSTYLYNRQRKIKKYRL

SEQ ID NO: 760 - protein, 326 aa, synthetic construct
  REGION 1..326: note = HA-CD3.s.004.01-G4Sx3-ICAM1
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKGGGGSGGGGSGGGGSIVIITVVAAAVIMGTAGLSTYLYNRQRKIKKYRL

SEQ ID NO: 761 - protein, 317 aa, synthetic construct
  REGION 1..317: note = HA-CD3.s.004.01-AEAAAK-ICAM1
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKAEAAAKIVIITVVAAAVIMGTAGLSTYLYNRQRKIKKYRL

SEQ ID NO: 762 - protein, 373 aa, synthetic construct
  REGION 1..373: note = HA-CD3.s.004.01-CD8ah-ICAM1
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKALSNSIMYFSHFVPVFLPAKPTTTPAPRPPTPAPTIASQPLSLRPEASRPAAGGAVHTRGLDIVIITVVAAAVIMGTAGLSTYLYNRQRKIKKYRL

SEQ ID NO: 763 - protein, 370 aa, synthetic construct
  REGION 1..370: note = HA-CD3.s.004.01-IgG3ser-ICAM1
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKELKTPLGDTTHTSPRSPEPKSSDTPPPSPRSPEPKSSDTPPPSPRSPEPKSSDTPPPSPIVIITVVAAAVIMGTAGLSTYLYNRQRKIKKYRL

SEQ ID NO: 764 - protein, 353 aa, synthetic construct
  REGION 1..353: note = HA-CD3.s.004.01-CD28-ICAM1
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKAAAIEVMYPPPYLDNEKSNGTIIHVKGKHLCPSPLFPGPSKPIVIITVVAAAVIMGTAGLSTYLYNRQRKIKKYRL

SEQ ID NO: 765 - protein, 370 aa, synthetic construct
  REGION 1..370: note = HA-CD3.s.004.01-IgG3hng-ICAM1
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKELKTPLGDTTHTCPRCPEPKSCDTPPPCPRCPEPKSCDTPPPCPRCPEPKSCDTPPPCPIVIITVVAAAVIMGTAGLSTYLYNRQRKIKKYRL

SEQ ID NO: 766 - protein, 334 aa, synthetic construct
  REGION 1..334: note = HA-CD3.s.004.01-IgG1hng-ICAM1
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKEPKSCDKTHTCPPCPAPEAAGGPIVIITVVAAAVIMGTAGLSTYLYNRQRKIKKYRL

SEQ ID NO: 767 - protein, 323 aa, synthetic construct
  REGION 1..323: note = HA-CD3.s.004.01-IgG4g-ICAM1
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKESKYGPPCPSCPIVIITVVAAAVIMGTAGLSTYLYNRQRKIKKYRL

SEQ ID NO: 768 - protein, 364 aa, synthetic construct
  REGION 1..364: note = RevisedHA-UMTxOKT3
GTMETDTLLLWVLLLWVPGSTGYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRSDPTTTPAPRPPTPAPTIASQPLSLRPEACRPAAGGAVHTRGLDFACDIYIWAPLAGTCGVLLLSLVITLYCNHRNRRRVCKCPRPVV

SEQ ID NO: 769 - protein, 352 aa, synthetic construct
  REGION 1..352: note = RevisedOKT3
GTMETDTLLLWVLLLWVPGSTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRSDPTTTPAPRPPTPAPTIASQPLSLRPEACRPAAGGAVHTRGLDFACDIYIWAPLAGTCGVLLLSLVITLYCNHRNRRRVCKCPRPVV

SEQ ID NO: 770 - protein, 358 aa, synthetic construct
  REGION 1..358: note = HA-ASGR1.s.017.02-IgG1ser-PDGF
ATMETDTLLLWVLLLWVPGSTGDYPYDVPDYAGGSEVQLVESGGGLVQPGGSLRLSCAASGFTFSSYNMNWVRQAPGKGLEWVSYISRSSNTKHYADSVKGRFTISRDNAKNSLYLQMNSLRDEDTAVYYCARDRSGSYGYFYYYGLDVWGQGTTVTVSSGGGGSGGGGSGGGGSQSVLTQPPSASGTPGQRVTISCSGSSSNIGSNAVNWYQQLPGTAPKLLIYSNNQRPSGVPDRFSGSKSGTSASLAISGLQSEDEADYYCAAWDDSLNGVVFGGGTKLTVLEPKSSDKTHTSPPSPAPEAAGGPNAVGQDTQEVIVVPHSLPFKVVVISAILALVVLTIISLIILIMLWQKKPR

SEQ ID NO: 771 - protein, 263 aa, synthetic construct
  REGION 1..263: note = pCAGGS-SS-GGlandingpad-RFP
ATMETDTLLLWVLLLWVPGSTGDGDGMVSKGEELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRSDMALKLVGGGHLICNFKTTYRSKKPAKNLKMPGVYYVDHRLERIKEADKETYVEQHEVAVARYCDLPSKLGHKLN

SEQ ID NO: 772 - protein, 352 aa, synthetic construct
  REGION 1..352: note = HA-CD3.s.001.01-IgG4PDGFR-Dob
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSESKYGPPCPSCPSACRDLKRCPRELPPTLLGNSSEEESQLETNVTYWEEEQEFEVVSTLRL

SEQ ID NO: 773 - protein, 303 aa, synthetic construct
  REGION 1..303: note = HA-CD3.s.001.01-IgG4PDGFR-Dob2
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSESKYGPPCPSCP

SEQ ID NO: 774 - protein, 342 aa, synthetic construct
  REGION 1..342: note = HA-CD3.s.001.01-EAAAKx3-ICAM1
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSAEAAAKEAAAKEAAAKAIVIITVVAAAVIMGTAGLSTYLYNRQRKIKKYRL

SEQ ID NO: 775 - protein, 442 aa, synthetic construct
  REGION 1..442: note = HA-CD3.s.004.01-IgG1ser-CD8aTm
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKEPKSSDKTHTSPPSPAPEAAGGPIASQPLSLRPEACRPAAGGAVHTRGLDFACDIYIWAPLAGTCGVLLLSLVITLYCNHRNRRRVCKCPRPVVIASQPLSLRPEACRPAAGGAVHTRGLDFACDIYIWAPLAGTCGVLLLSLVITLYCNHRNRRRVCKCPRPVV

SEQ ID NO: 776 - protein, 328 aa, synthetic construct
  REGION 1..328: note = HA-CD3.s.004.01-EAAAKx3-ICAM1
ATMETDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKAEAAAKEAAAKEAAAKAIVIITVVAAAVIMGTAGLSTYLYNRQRKIKKYRL

SEQ ID NO: 777 - protein, 356 aa, synthetic construct
  REGION 1..356: note = HA-CD3.s.001.01-G4Sx3-PDGFR
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSGGGGSGGGGSGGGGSNAVGQDTQEVIVVPHSLPFKVVVISAILALVVLTIISLIILIMLWQKKPR

SEQ ID NO: 778 - protein, 403 aa, synthetic construct
  REGION 1..403: note = HA-CD3.s.001.01-CD8ah-PDGFR
ATMETDTLLLWVLLLWVPGSTGDYPYDVPDYAGGSAAQPASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGGGSGGGGSGGGGSVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAARGSALSNSIMYFSHFVPVFLPAKPTTTPAPRPPTPAPTIASQPLSLRPEASRPAAGGAVHTRGLDNAVGQDTQEVIVVPHSLPFKVVVISAILALVVLTIISLIILIMLWQKKPR

SEQ ID NO: 779 - protein, 321 aa, synthetic construct
  REGION 1..321: note = HA-CD3.s.004.01-IgG4-ser-ICAM1
METDTLLLWVLLLWVPGSTGDYPYDVPDYAGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIKESKYGPPSPPSPIVIITVVAAAVIMGTAGLSTYLYNRQRKIKKYRL

SEQ ID NO: 780 - protein, 5 aa, synthetic construct
  REGION 1..5: note = peptide linker
  REPEAT 1..5: note = repeat 1 to 10
EAAAK

SEQ ID NO: 781 - protein, 62 aa, synthetic construct
  REGION 1..62: note = CD8ah
ALSNSIMYFSHFVPVFLPAKPTTTPAPRPPTPAPTIASQPLSLRPEASRPAAGGAVHTRGLD

SEQ ID NO: 782 - protein, 34 aa, synthetic construct
  REGION 1..34: note = ICAM1
IVIITVVAAAVIMGTAGLSTYLYNRQRKIKKYRL

SEQ ID NO: 783 - protein, 57 aa, synthetic construct
  REGION 1..57: note = IgG3 hinge
LKTPLGDTTHTCPRCPEPKSCDTPPPCPRCPEPKSCDTPPPCPRCPEPKSCDTPPPC

SEQ ID NO: 784 - protein, 21 aa, synthetic construct
  REGION 1..21: note = IgG1 hinge
PKSCDKTHTCPPCPAPEAAGG

SEQ ID NO: 785 - protein, 51 aa, synthetic construct
  REGION 1..51: note = PDGFR
PNAVGQDTQEVIVVPHSLPFKVVVISAILALVVLTIISLIILIMLWQKKPR

SEQ ID NO: 786 - protein, 49 aa, synthetic construct
  REGION 1..49: note = VSVGan
SSIASFFFIIGLIIGLFLVLRVGIHLCIKLKHTKKRQIYTDIEMNRLGK

SEQ ID NO: 787 - protein, 126 aa, synthetic construct
  REGION 1..126: note = CD8aTm
IASQPLSLRPEACRPAAGGAVHTRGLDFACDIYIWAPLAGTCGVLLLSLVITLYCNHRNRRRVCKCPRPVVIASQPLSLRPEACRPAAGGAVHTRGLDFACDIYIWAPLAGTCGVLLLSLVITLYC

SEQ ID NO: 788 - protein, 39 aa, synthetic construct
  REGION 1..39: note = HLADRB
MLSGVGGFVLGLLFLGAGLFIYFRNQKGHSGLQPTGFLS

SEQ ID NO: 789 - protein, 33 aa, synthetic construct
  REGION 1..33: note = CD49d
IVIISSLLLGLIVLLLISYVMWKAGFFKRQYKS

SEQ ID NO: 790 - protein, 11 aa, synthetic construct
  REGION 1..11: note = IgG4
ESKYGPPCPSC

SEQ ID NO: 791 - protein, 41 aa, synthetic construct
  REGION 1..41: note = CD28
AAAIEVMYPPPYLDNEKSNGTIIHVKGKHLCPSPLFPGPSK

SEQ ID NO: 792 - protein, 12 aa, synthetic construct
  REGION 1..12: note = IgG4PDGFR
ESKYGPPCPSCP

SEQ ID NO: 793 - protein, 31 aa, synthetic construct
  REGION 1..31: note = GPI
PQLENGGTSLSEKTVLLLVTPFLAAAWSLHP

SEQ ID NO: 794 - protein, 110 aa, synthetic construct
  REGION 1..110: note = Transferrin
MMDQARSAFSNLFGGEPLSYTRFSLARQVDGDNSHVEMKLAVDEEENADNNTKANVTKPKRCSGSICYGTIAVIVFFLIGFMIGYLGEPKSSDKTHTSPPSPAPEAAGGP

SEQ ID NO: 795 - protein, 562 aa, synthetic construct
  REGION 1..562: note = BaEVTR
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLVQFVKDRISVVQALVLTQQYHQLKPLEYEP

SEQ ID NO: 796 - protein, 546 aa, synthetic construct
  REGION 1..546: note = BaEVRLess
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAM

SEQ ID NO: 797 - protein, 562 aa, synthetic construct
  REGION 1..562: note = BaEVTR + protease mod
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLVQFVKDRISVSQNYPIVQQYHQLKPLEYEP

SEQ ID NO: 798 - protein, 545 aa, synthetic construct; identical to SEQ ID NO: 622
  REGION 1..545: note = BaEV

SEQ ID NO: 799 - DNA, 1164 bp, synthetic construct
  source 1..1164: mol_type = other DNA
  misc_feature 1..1164: note = PLAS3310 -- HA-RNlinker-F-IgG1ser-GPI
atggagactgacacactgctcttatgggtgcttctgttgtgggtccccggcagcaccggggattacccctacgatgtgcctgactatgccggaggaagtgaaatattggacgtgcccagcaccggaggagggggttcgggagggggaggcagtggagggggcggcagtcctacagatctgcgtttcaccaatatcggtcctgacacaatgcgggtgacttgggcaccaccgccgagcattgatttgacaaattttctggttaggtacagccctgtgaagaacgaggaggatgtggcagagctgtcaatcagcccatccgacaatgccgttgtgcttaccaacctgctacccgggacagaatatgtggtctccgtgagtagtgtatacgagcaacacgagagcactccccttagaggtaggcagaagacaggtctggactcccccaccgggattgacttcagcgacattacggcgaactcttttactgtacattggattgcacctcgcgcaactatcaccggctacagaattagacaccacccagaacatttcagcggcaggcccagggaggatcgcgtccctcactctcgcaactctatcaccctcactaatctgacaccaggcactgaatacgtggtctccatagttgctctcaatggacgggaagagtcccccctgctgattggccagcagagcacggtgtccgatgtccctcgtgatctggaggtcgtcgctgctaccccaacctctctactcatatcatgggatgccccagctgtgacagtgcgatattaccggattacctatggcgagacaggcggaaactccccggtccaggaatttactgtgcccggcagtaagtctactgccacaatttcgggacttaagcccggggttgactacaccatcacggtctacgccgtgacgggacggggcgactcccctgcctcttcaaagcctatctctattaactatagaacagaaatcgacaaaccgagcgagcccaagtctagcgacaaaacccacacaagtccaccttcccctgccccagaagctgcaggcggaccccagcttgagaacgggggcacatctctctccgaaaagactgtcctgttactggtgacccctttcttggctgccgcatggagcctgcacccctgataa

SEQ ID NO: 800 - protein, 386 aa, synthetic construct
  REGION 1..386: note = PLAS3310 -- HA-RNlinker-F-IgG1ser-GPI
METDTLLLWVLLLWVPGSTGDYPYDVPDYAGGSEILDVPSTGGGGSGGGGSGGGGSPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSEPKSSDKTHTSPPSPAPEAAGGPQLENGGTSLSEKTVLLLVTPFLAAAWSLHP

SEQ ID NO: 801 - protein, 240 aa, synthetic construct; identical to SEQ ID NO: 501
  REGION 1..240: note = CD8 scFv

SEQ ID NO: 802 - protein, 242 aa, synthetic construct
  REGION 1..242: note = CD3 scFV
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSDIVLTQSPAIIVISASPGEKVTMTCSASSSVSYMNWYQQKSGTSPKRWIYDTSKLASGVPAHFRGSGSGTSYSLTISGMEAEDAATYYCQQWSSNPFTFGSGTKLEIK

SEQ ID NO: 803 - protein, 396 aa, synthetic construct
  REGION 1..396: note = IgG4 heavy chain seq
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLELQLEESCAEAQDGELDGLWTTITIFITLFLLSVCYSATVTFFKVKWIFSSVVDLKQTIVPDYRNMIRQGA